SEQUENCE LISTING (159 sequences)

SEQ ID NO: 1 - DNA, 1664 bp, Danio rerio
tcatccgtgggcgttgggggctgctgtttacagacccgagcgggcggtttgtttcgagcatgaaggactgcgcttttttcaccctaccgccattcattcaggaattatatctgaactcgagtaaaccagaaggttttaatcaaagtgcttcagccggactgtttttgcaaacgcttttcctgcacagacattctgaaccgcgtcgcgcgaagttggaccatctgttgaacgtttgcgagcgcaccgcaaagttctcaacgcgtttattttttaacatttaatcgcaaaacaactgttgttgtttcctaaagcgaacgtttttgttgcgtttatgctaacgccaagcaactttggagaggttagctatagctggttagcctagactcgctaactggcctctgctaaaagggccgttagtgacctaaacgtattgttgttctacatttattcataacgtgtaataataataactacaaagtttgttttctaatggtctctgtacgttatatttaactaaaacagttgcatttatgagtcgacaacttgagcagttgtgaaaagcattagcactccaaaaaacttcagttaaacattttccttaaggaattaacgtaagcattctctctgttttgctatacagcaagagtgacctccatcattggccatatgtaagacgaaccagtccttttaaaacccaatattctcaaagctggctgaaccgccagctaaaagtaacttaaatcgcaacttttgctcgtaaatgaccaccggcaggaataaccgagtgatgatggaaggagtgggtgctcgagttatccgcgggcctgactggaagtgggggaagcaggacggcggagaagggcatgtgggcaccgtcaggagcttcgagagcccggaggaggtggtggtggtgtgggataacggcaccgccgccaattaccgctgctccggggcttatgatgtgaggatattagacagtgcgcctacaggaatcaagcatgatggcaccatgtgtgacacctgtcggcagcagcccatcattggaatccgatggaaatgtgcagagtgcaccaactacgacctctgtaccacctgttaccatggcgacaagcaccacctgcgccacaggttttaccgcatcaccacccctggcagtgaaagagttcttctggagtctcgtcgcaagtcgaagaagatcacagcccgcggcatctttgcaggaggacgagtggtgcgaggggtggattggcaatgggaggatcaggatggcggaaatggaagaagagggaaggtgacggagatccaggactggagtgctgccagcccccacagcgcagcctacgtcctttgggacaacggggccaagaacctctaccgagtgggattcgagggcatgtctgatttgaaatgtgtgcaagatgccaaaggagggactttttacagagaccactgtccagtattgggtaagatttaaaatatctgatattaccaaaatataccatgccgattcaatgaatatacagtgggggaaataagtattgagcacgtcaccatttttctcagaaaacaaatttctaaaggtgctgttgacttgacttttcaccagatgttggtaacaaccaaagaaatccatatatgccaaaaaaaaaaaaaaaaaaaaaaaa

SEQ ID NO: 2 - protein, 237 aa, Danio rerio
MTTGRNNRVMMEGVGARVIRGPDWKWGKQDGGEGHVGTVRSFESPEEVVVVWDNGTAANYRCSGAYDVRILDSAPTGIKHDGTMCDTCRQQPIIGIRWKCAECTNYDLCTTCYHGDKHHLRHRFYRITTPGSERVLLESRRKSKKITARGIFAGGRVVRGVDWQWEDQDGGNGRRGKVTEIQDWSAASPHSAAYVLWDNGAKNLYRVGFEGMSDLKCVQDAKGGTFYRDHCPVLGKI

SEQ ID NO: 3 - DNA, 2361 bp, Danio rerio
gattggcccattctgattgatggccgccacacaaggactgcgcacatttccacacaggctgatactgagacgcgcaatctgagtgtagtctgtttggatcatcctgggatggagcaactgatcgacgtttattgattatcaaaccagcagtctttacgctttcttgtttgaaatctcaacaacctttttagcggaaagatgacggagagagcgcagagcccaacagcagcagactgcagaccctatgaggtcaacagggccatgtatcctcaagccgcgggcctggatggacttggcggagcgtccttgcagtttgcgcacggtatgcttcaggatccaagtctgatttttaacaaggcccatttcaacggaatcacccccgcgacagcccagaccttctttccattttcaggcgattttaaaacgaacgatttgcaaggtggcgactttacgcagcccaaacactggtacccgtttgcggcccccgagttcactgggcaggttgcaggagcgactgccgccactcagccggcgaacatcagccctcctatcggcgagactagagagcaaattaagatgccatctgaggtcaaaaccgagaaagatgttgaagaatacgggaatgaagaaaacaagccgccgtcacaatatcacctcaccgctggaacatcttccatccccaccggggtgaactactacacgccatggaaccctaatttctggcctggactgtcccaaattacggcccaagctaatatttcccaagctcccccaactccctccgcttcatccccatctctgtctccgtctccccctggaaatgggttcggaagcccaggattttttagcggaggcaccgcgcaaaacattccctcagctcaagcgcaaagtgcaccccggagcagtgggtcctccagtggaggatgcagtaattctgaggaagaggagactctgactactgaagatttggagcagtttgcgaaagagctcaaacacaagcgcatcactctgggcttcacgcaggcagatgtgggactcgcgcttggaaacttgtatggcaaaatgttcagtcagacgacaatctgccgctttgaggctctccaacttagtttcaagaacatgtgcaaactgaagccgctgctgcagaggtggttgaacgaggccgaaaactccgagaaccctcaggatatgtacaaaattgaacgggtgtttgtcgacacgcgaaaaagaaaacggaggaccagcttggaaggcacagtccgttctgctctagagtcgtacttcgtgaagtgccccaaacccaacactctggagataacgcacatatcggatgatctaggcctggagagagatgtagtgcgtgtatggttctgcaaccgtagacagaagggaaagcgtctagctttgccctttgatgacgagtgtgttgaagcacagtattacgagcagagtccaccacctccaccccacatgggtggcactgtgctcccaggtcaaggctatcctggaccagcccatcctggaggagcccctgccttatacatgccatccctccaccgaccagatgtcttcaaaaacggctttcaccctggtttggtgggtcatctcaacagctaacttgctccaggtgctcactaaagtttatagaaaagctccaagccaagctatggatcaaatcagccatgcttataattcttttaattttattttgtaacactgcatctgttcatttggttctttccatacgtgccttgagacacttatgttagtgtctggttgaccatcacagtaggaacctgatggattattgtgttatgagtgctttgttgaactactgcgtttgcacatgaagtcataggctcatttttatagttgtttttaacaatgtttgctcatgtatcggtttttatgtacatatatttgtcttaaacgtttgatatttgatgatttaccaaagtatatttccatatttgtctccataactttgtagttttcccgtatttgtgtgaactggcagcaaattcaagacttgatgaaatttaaatttgactttctttttcccttttttttttttttaattttaatgacgtgatggattttttttttattacgatttggagaacttttatcaaatgatttgacaagaacattagtcactgttctttttcacctctctaaatgcttgcactctcccatctccaagtgaaatgcctctagatgtactctttatcagggtggttatacataaatatgctgccagtttgaaagcctctctgtggaggtcatgtgccttatctttcaatgtctcaattcatgcatgctcacccgcagtaaattaaagccatatttgataatttattg

SEQ ID NO: 4 - protein, 472 aa, Danio rerio
MTERAQSPTAADCRPYEVNRAMYPQAAGLDGLGGASLQFAHGMLQDPSLIFNKAHFNGITPATAQTFFPFSGDFKTNDLQGGDFTQPKHWYPFAAPEFTGQVAGATAATQPANISPPIGETREQIKMPSEVKTEKDVEEYGNEENKPPSQYHLTAGTSSIPTGVNYYTPWNPNFWPGLSQITAQANISQAPPTPSASSPSLSPSPPGNGFGSPGFFSGGTAQNIPSAQAQSAPRSSGSSSGGCSNSEEEETLTTEDLEQFAKELKHKRITLGFTQADVGLALGNLYGKMFSQTTICRFEALQLSFKNMCKLKPLLQRWLNEAENSENPQDMYKIERVFVDTRKRKRRTSLEGTVRSALESYFVKCPKPNTLEITHISDDLGLERDVVRVWFCNRRQKGKRLALPFDDECVEAQYYEQSPPPPPHMGGTVLPGQGYPGPAHPGGAPALYMPSLHRPDVFKNGFHPGLVGHLNS

SEQ ID NO: 5 - DNA, 543 bp, Danio rerio
taaaaaaaaaaaaaaaaaaaaaaactcaagatgtccctcgtcatcccagagaagtttcagcacatccttcgtgtcctcaacacgaacattgatggaagacgtaaaatcgcatttgccatcaccgctattaagggtgttggaaggcgatatgcccatgtggttctgaggaaagctgatattgatttaaataagagggctggagaactcactgaggatgaggttgagagggtggtgactattatgcagaaccctcgccagtacaaaatcccagactggttcctgaacagacagaaggacataaaagatgggaaatacagccaggtccttgctaatggtctggacaataaactgagagaagatctggagaggctgaagaagatcagggctcaccgtggtctccgtcacttctggggtctgcgtgtgcgtggtcagcacaccaaaacaactggtcgtcgcggtcgcactgtgggtgtgtccaagaagaagtaaatttacaaataaaggaaatcattcagtaaaaaaaaaaaaaaaaaaaaaaaaaaa

SEQ ID NO: 6 - protein, 152 aa, Danio rerio
MSLVIPEKFQHILRVLNTNIDGRRKIAFAITAIKGVGRRYAHVVLRKADIDLNKRAGELTEDEVERVVTIMQNPRQYKIPDWFLNRQKDIKDGKYSQVLANGLDNKLREDLERLKKIRAPRGLRHFWGLRVRGQHTKTTGRRGRTVGVSKKK

SEQ ID NO: 7 - DNA, 938 bp, Danio rerio
aatggccgaatacttggcgtctattttcggtacagaaaaagacaaggtcaactgctctttctactttaaaattggagcctgtcgccatggagaccgatgctctagacttcataataaacccaccttcagtcagaccattgccctgttgaacatctacaggaacccacaaaacacagcccagtctgctgatggcttaaatgctgtcagtgatgtggagatgcaggaacattatgatgagttctttgaggaggtcttcactgaaatggaggagaaatatggagaggttgaggagatgaacgtctgcgataatttaggagatcacttggtgggaaatgtctatgtgaagttccgccgtgaagaagatgcagagaaagcggtgattaacctgaacaaccgctggtttaatgggcagcccattcatgcggagctctcgcctgtcactgacttcagagaggcctgttgtcgacagtatgagatgggagagtgcactcgaggaggcttctgcaatttcatgcatctgaagccaatctcaagggaactaaggagagagctgtacggccgcagaaggaagagacatcggtcccgctctcgctcccgtgaacgacgctctcgctctaggggacgcaataaaggagttggtggtgcagcagcaggcggtggtggtggcggcggtggtggtggtggaggtggaggaggaagagaccgagagagacggcggtcaagagatagagagcgttctggaagattttaaaatttccttctttcttgaaatcaacattcagtcaccagacccttcagctctctccctgatgaaaatgtgataactgttgaaattgtttccttctttaattgtcctctgatctttttattcaacgtttgtttgcttttcaataaacaaacaccttagtcgtttcaaaaaaaaaaaaaaagggcggccg

SEQ ID NO: 8 - protein, 249 aa, Danio rerio
MAEYLASIFGTEKDKVNCSFYFKIGACRHGDRCSRLHNKPTFSQTIALLNIYRNPQNTAQSADGLNAVSDVEMQEHYDEFFEEVFTEMEEKYGEVEEMNVCDNLGDHLVGNVYVKFRREEDAEKAVINLNNRWFNGQPIHAELSPVTDFREACCRQYEMGECTRGGFCNFMHLKPISRELRRELYGRRRKRHRSRSRSRERRSRSRGRNKGVGGAAAGGGGGGGGGGGGGGGRDRERRRSRDRERSGRF

SEQ ID NO: 9 - DNA, 2166 bp, Danio rerio
agcggaatcatcgcttatgttatcctgctccgagaagctgatatggagtcgctttattatatttaagacactgctggactcttgattaataatactggattatcaatcaaccttgaccgttggacaagggaggatttaatcagaagcgattgttattgcgtagcattttggtaacttggatagttagcatctttaagctgcaagtctagagtcatgtcgctaaattgctaaaggacacatttcatatctacccaccaaaatgccgtttttcttcctttaaccgataaacttggattaatgtggactaaagccgaaaataaactgcccagactactttaactgtcggatgctttttgacgcagtaatagttagtaaataagttagcattgctagcatagtcgtgcagccgctcttcaaatagaaacttccgtgttgcactacgaaagtccgttatcgtatgtttttggggctgtaaacccgaagcgagtcttcctcgtgttttatttgcaagtatcaggcttgaatgtgaactagcgtgagatgcggtggtaaagaagagttcaatgccgtgtgtgctggaggatgatgcggatgagctggatggtcacagtcattaacagaagaatgatgaagatcctgatcgccctcgctctcattgcgtacatcgcctctgtctggggaacatacgcaaatatgaggtctatacaggaacacggagaaatgaagattgagcagaggatagatgaggcagtgggacctctcagggagaagatacgggaactcgagcttagttttacccagaagtacccaccagtgaaattcttatccgagaaggaccgaaagaggattttgatcaccggtggggcaggattcgtgggctcccacctcaccgataagctgatgatggatggtcatgaggtgactgtggtggataatttcttcaccggccgcaagcgcaatgtagagcactggattggccatgagaacttcgagctcatcaaccatgatgtggtggagccgctctatattgaagttgaccagatatatcatttggcctcgcctgcatcgccaccgaactacatgtacaatccgatcaagacactaaaaacaaatactattggtactctcaacatgctaggattggccaagcgggttggagcgcgcctgcttcttgcctccacgtcagaagtgtacggagacccagaggtgcacccccaaaatgaggactactggggtcacgtgaatccaattggtccccgggcctgttatgatgaaggaaagcgtgttgctgagactatgtgttatgcctacatgaaacaggaaggagtggaggtgcgagtggctcgcatcttcaacaccttcgggtctcgcatgcacatgaacgacgggcgagtggtcagcaatttcatcctgcaggctttgcaaggagaggcactgacggtctacggttcagggtctcaaacaagagctttccaatatgtgagtgacctggtgaatggactggtgtctctgatgaacagtaacatcagtagccctgttaacctgggaaatccagaggaacacaccatcttggagtttggcagctcatcaagagtcttgttgcgagccggagtcatattcagttcctttcagaagcgcaggatgatccacagaggagacggaccgacatccgcaggccaaactgctgctgggctgggaacctgtggtcccactggaggaaggcttgaacaaaaccatccagtacttcagccgagagctggagcatcaagccaacaatcagtacatccccaaacccaaagctgcccgcatgaaaaaaggaagacccagacacaactgaagacacggcttcatctcagggatgctgggatatggaggacttcagacatccggcagccactttaccaactcccactaacagatgcgtcgttttggcagagaggggataagagaagtttttctgatgtttttcagtcgtatgtgcctcaaaatgatccaatttacagactttgccttgcactttggtttgtgagtcgtctgtctgtctgtgtgtgtgtctgttatttaaagcaagcttaggtcttcatgtgcgttatgtttacattctgtgctgttttaattgatgtgatcagtttttttttttggattatcattgaagctaaaggtggg

SEQ ID NO: 10 - protein, 497 aa, Danio rerio
MMRMSWMVTVINRRMMKILIALALIAYIASVWGTYANMRSIQEHGEMKIEQRIDEAVGPLREKIRELELSFTQKYPPVKFLSEKDRKRILITGGAGFVGSHLTDKLMMDGHEVTVVDNFFTGRKRNVEHWIGHENFELINHDVVEPLYIEVDQIYHLASPASPPNYMYNPIKTLKTNTIGTLNMLGLAKRVGARLLLASTSEVYGDPEVHPQNEDYWGHVNPIGPRACYDEGKRVAETMCYAYMKQEGVEVRVARIFNTFGSRMHMNDGRVVSNFILQALQGEALTVYGSGSQTRAFQYVSDLVNGLVSLMNSNISSPVNLGNPEEHTILEFGSSSRVLLRAGVIFSSFQKRRMIHRGDGPTSAGQTAAGLGTCGPTGGRLEQNHPVLQPRAGASSQQSVHPQTQSCPHEKRKTQTQLKTRLHLRDAGIWRTSDIRQPLYQLPLTDASFWQRGDKRSFSDVFQSYVPQNDPIYRLCLALWFVSRLSVCVCVCYLKQA

SEQ ID NO: 11 - DNA, 1399 bp, Danio rerio
ctgattgagggttaagagcaagacagacgcgtgccattatcttcaacacaaactatctgcaggattctgcaaaacctcaagcatctcccagcccaccaataaggttatcaacaatggagatcgtatactccgatatggaaacctcaagctgtgactactccttttcgcacacggatgatgaagactcgcgcagcagcctccaccccgcgtcccccgcgtcctcctgcggaaaaccacctgcgtctccagccgggctccagcagaagaaaaggcgcagggggcgcgcgaggaacgaaaccactgtgcacgtcgtgaagaagaaccgcaggctgaaggccaacgaccgcgagaggaacaggatgcacaaccttaacgacgcattggatgctttgagaagcgtcctgcctgcgtttcctgacgacacaaagctgaccaaaattgagactctgcgcttcgctcacaactacatctgggcactttcggagaccatccggatcgcagaccagaagcagggcaagtcaagagacggtccgctgctgctccccggactaagctgcatggcagatgcacccagccccggcagtgactcttgctcctggccgtcgggggcatcctcgtcgtcttcatcaccgtcttactgcaactcagacccgggcagccccgcagccatggacgattttggatacttgcaaaccgacgtagtgtacagctaccgcaacttcgtgcctagcatctattaatgtgactttaagcgttctcatgtcggtatagaatctccagaagtatgtaatagtgtaagaccactgttacttttctattagaaccaagagcacaacgttaggtattcactgtttgccttaatgacagtctgcaaatgagaatgttttatttttgtacgttttgatattgatgttgatgttgatgcttttttccaatacctttgcactttttgcattccttggagcgtgaccagaaccccgttagagaaaattataatttaaaaagcaaggcagattggcctttgctgtccaaggtgaaaagtcaatattacaatttgtagagcgataaagctgaaaaacgagcgtgaaaaatcagtttcaacgctgtgacaatagactgaaaggacaagacaaagatctggccttatgcattatgtattttgacccgaagaaaccctcatctgtccgactgatccgtacagttaaccagctctcatgacggctttcccaccacttttctttttttatttattcaacctgttgcttctttatgctattgtatttttattgtatataaagagatttattctattatgtatttacctcagaatataaatctggtgcaaacatgtttgtacgaataaatatacgttttctactaaaaaaaaaaaaaaaaaaaa

SEQ ID NO: 12 - protein, 208 aa, Danio rerio
MEIVYSDMETSSCDYSFSHTDDEDSRSSLHPASPASSCGKPPASPAGLQQKKRRRGRARNETTVHVVKKNRRLKANDRERNRMHNLNDALDALRSVLPAFPDDTKLTKIETLRFAHNYIWALSETIRIADQKQGKSRDGPLLLPGLSCMADAPSPGSDSCSWPSGASSSSSSPSYCNSDPGSPAAMDDFGYLQTDVVYSYRNFVPSIY

SEQ ID NO: 13 - DNA, 1587 bp, Danio rerio
agctccttttggactattacacttccagataaatagtattgaagaaaaagtttttgagtcgggccttggatccaagtgatatttggcttgatgcatggctggcatgttgccttaaaaaaagttattttaggggtttcttccaaatcatgtacgttggataccttttggagaaagaggcaagcatgtatcaccaaggagcggttcgtcgatctggcatcagtcttccaccacagaactttgtttccacacctcagtattcagattttacaggataccatcatgtgcccaatatggagacacacgcacagtcggcaggggcatggggcgctccttatggcgctccgagagaggactggggcgcctacagcctcggacctccaaattctatttctgcacctatgagcaattcatctccgggaccagtttcctattgctcgcccgattataacaccatgcacggccctggatcggcggttcttcctccgccacctgaaaatattcctgtggcccaactatccccagagagagaaaggcgtaattcctatcagtggatgagcaaaaccgtccagtcgtcatcaaccggcaaaacgagaacgaaggagaaataccgagtggtatacacagatcaccaaaggctagagctggagaaagagtttcattttaatcgctatatcacaatcagaagaaaatccgaacttgctgtaaacctcgggctttcagagagacaggttaagatctggtttcagaaccgcagagctaaggaaaggaaattaatcaaaaagaaattgggtcagtctgacggaagcggagggtcagtgcacagcgacccaggctccgtcagtcctctaccggtaagggtcactgagtccttcagatatacacggttctctgtatccagcacaaatgaacgccttgccgtccatgaggaacatacagcaagttactgtcactcagtgaactcgaacttggatcatccaactttaactgagcactccaacacgctgaaaggacagaaccaaacgtggtcaaaaaggattacaaactattaatgaataaatgaatgaataaataaataagaggcgtgggttgtggattacaaccgaagaatcacgtctcaaatgcgacgaaatgctggaggctcttgaaatattcttttaatagaatagtttttaaaattaagaaaaaaaaactacaaaaaccttgttcgtctgatccagagactaaataggctattttaatggtttgtgttttctcgaataagatataaatatcttttttctgtatgtggtgagtgtatctaaatgtttgtttgtatataaaatgagaactacaggtacggagtttcatggacgtgtaatgatccttaccctggaactgttaaacttttattgtctgtgaaaaccaatgacctgcgatgtcaggctgttttatgcgccagtttgtgtccaaagcgcacgtcgttcactttcttttcacaggggtgatgtgcaggtgtcacataccagatacgggtataacgtttgatttcagcaatgtgtaaataaatgttttgcaataacttttattgtgtcttcatgtatcattaaa

SEQ ID NO: 14 - protein, 300 aa, Danio rerio
MYVGYLLEKEASMYHQGAVRRSGISLPPQNFVSTPQYSDFTGYHHVPNMETHAQSAGAWGAPYGAPREDWGAYSLGPPNSISAPMSNSSPGPVSYCSPDYNTMHGPGSAVLPPPPENIPVAQLSPERERRNSYQWMSKTVQSSSTGKTRTKEKYRVVYTDHQRLELEKEFHFNRYITIRRKSELAVNLGLSERQVKIWFQNRRAKERKLIKKKLGQSDGSGGSVHSDPGSVSPLPVRVTESFRYTRFSVSSTNERLAVHEEHTASYCHSVNSNLDHPTLTEHSNTLKGQNQTWSKRITNY

SEQ ID NO: 15 - DNA, 1269 bp, Danio rerio
ccagtctattttcacggctctgacactgtcacgatcaacccttttcgagtaaatttaaaatctaaaggctttcaggattattttcgagctgtatttgcaaagcagacgcctttcctctgccgcaaaccgagctttatctctcggcggagtttaaaatccccttaaagcagcaggagcaggatctgcagtcatggcgctcagcgatgccgacgtccagaaacagatcaagcacatgatggctttcattgagcaggaggccaatgaaaaagccgaggagatagacgccaaggcggaggaggagtttaacattgagaaaggtcgactggttcagacccagcgcttgaagatcatggagtattacgaaaagaaggagaaacagatcgaacagcagaagaaaattcaaatgtccaacctgatgaatcaggccagactgaaggtcctgaaggccagagatgacatgattgcggatttactaaacgacgcacgtcagcgactagcgaatgtagccagagatccgtccagatacgcggctctgatggacggactggttctgcagggtttttaccagctgctggaacctaaagtgaccatccgttgccgcaaacaggatgtggggattgtgcaggccgccgtgcaaaaaaacatctccatctacaaagcagcagtaaagaacaatctagaagtgcgcatcgaccaagacaacttcctctctccagaaatctctggaggtattgagctctataatgcggacgggaaaattaaagtggcaaacactctggagagccgactggaactcatcgcacagcacatgatgcctgaaataccagtcgccctgtttggtgcgaaccagaaccgcaagttcatggactgatggatctttccggagatcggcagtgttttgatgttgtatttttattttgatgttaaaaatccattcctgcgtctctatctgtactgtatttaagaagacaacacctgtgtgtgtgtgtgtgtgtgcgtatgattcatcattgctgtataaagatcaaaatagtgtcctgcacatttctgtgtttgattatgaataaattaaagctccgttatgttcctataaagtcttaaagtgatgcataaccctcgcttcatgaagaaaatatgactttttttttatttgcattcctgtaatgaggagcatgatctgtatatctgaagcaaacgtgtctgtcgatgatgcaaagagactatattgatgtgaaaactataatgaaaacccatatggcccagaaaaaa

SEQ ID NO: 16 - protein, 226 aa, Danio rerio
MALSDADVQKQIKHMMAFIEQEANEKAEEIDAKAEEEFNIEKGRLVQTQRLKIMEYYEKKEKQIEQQKKIQMSNLMNQARLKVLKARDDMIADLLNDARQRLANVARDPSRYAALMDGLVLQGFYQLLEPKVTIRCRKQDVGIVQAAVQKNISIYKAAVKNNLEVRIDQDNFLSPEISGGIELYNADGKIKVANTLESRLELIAQHMMPEIPVALFGANQNRKFMD

SEQ ID NO: 17 - DNA, 1740 bp, Danio rerio
ggccttgttgcaatttgacagcagaggaagcagctccttaaatataaaatctgaagatggacatccgaggggcagtggacgctgccgtccccaccaatatcattgcagcgaaggccgcagaagtccgagcaaacaaggtcaactggcagtcctaccttcagggccaaatgatctccggcgaggactgcgagttcattaagaagtttgaggttgctggatctgaggacaagcaagcaattctgacaaacgaaggacatcagtgtgcaaagaccttcctcaaccttatggctcatatatctaaggagcaaaccgttcagtacatcctgaccctgattgatgacacactgcaggaaaatcatcagagagtgaatatcttctttgattatgcaaagaagactaagaatacagcctggtcatacttcctcccaatgctgaaccgtcaagagctcttcactgttcacatggctgccagaatcatagccaaactggctgcctggggacgtgacctgatggaaggcagtgacttgaactactacttcaactggattaagacccagctcagctctcagagctcccagtacgttcagtgtgtcgctggctgcctacagttgatgttgagagtcaatgaatacagatttgcctgggtggaggccgatggagtgaactgcatcacggcagtgctgagcaataagtgtggcttccagctgcagtatcagatgatcttctgcgtgtggctcctggcattcagcccgcagctctgcgaacagctgcggcgctacaacgtggtaccagccctctccgacatcctccaagagtctgtcaaagagaaggtcactcgcataattctggctgctttcaggaacctgctggagaaatcagccgaaagagagactcgtcaagagtatgcgcttgccatgatccagtgcaaagtgctaaaacagctggagaacctggaccagcagaaatacgatgatgaggacatcaccgatgatattaaattcctcttggaaagactgggagagagtgttcaagatctcagttcgtttgatgagtacagctccgagctgaagtctggacgcctggaatggagtccagtgcacaagtcagagaagttctggagggaaaacgctgttcgcctgaatgagaagaactacgagctcctgaagatactgaccaggctgttggaagtgtctgatgaccctcaggttattgccgtcgcagctcacgacgtgggggaatacgtgagacattaccctcgcggcaagagggtgatcgaacagctgggtggtaaacaactagtgatgaaccacatgcatcatgaagaccagcttgtccgctataatgccctcctggccgtccagaagctcatggtgcataactgggagtacctggggaagcagttgcagtccaccgagcagacgactccggctgtggctgcacgaagctgaactgccctgcgtttctgtatgagcctgtgtgtctgatccagtgtcattggtgtgaatgtaaatctgtgttatatatataatgaacaaggtaaaccaaacggttgttgtattgatccacaaatttatgatttaactcttgtgcttttgtcgttgtgttattgtgtattgtgcgtcctaataataaaatcacgtcaagtgatcaggtcacagagtaaaatgaaatgttatttatggcttaagtgtaaaacgatgagtatgcattccaataaagaatctaccacaacaaaaaaaa

SEQ ID NO: 18 - protein, 463 aa, Danio rerio
MDIRGAVDAAVPTNIIAAKAAEVRANKVNWQSYLQGQMISGEDCEFIKKFEVAGSEDKQAILTNEGHQCAKTFLNLMAHISKEQTVQYILTLIDDTLQENHQRVNIFFDYAKKTKNTAWSYFLPMLNRQELFTVHMAARIIAKLAAWGRDLMEGSDLNYYFNWIKTQLSSQSSQYVQCVAGCLQLMLRVNEYRFAWVEADGVNCITAVLSNKCGFQLQYQMIFCVWLLAFSPQLCEQLRRYNVVPALSDILQESVKEKVTRIILAAFRNLLEKSAERETRQEYALAMIQCKVLKQLENLDQQKYDDEDITDDIKFLLERLGESVQDLSSFDEYSSELKSGRLEWSPVHKSEKFWRENAVRLNEKNYELLKILTRLLEVSDDPQVIAVAAHDVGEYVRHYPRGKRVIEQLGGKQLVMNHMHHEDQLVRYNALLAVQKLMVHNWEYLGKQLQSTEQTTPAVAARS

SEQ ID NO: 19 - DNA, 1439 bp, Danio rerio
ttcttcctctccgccggcgaccagcctcctgtcgggttcgtgaccgtctctcctgcttttgtaagacctgtcagccaaaagaagacctcactcagagtctaaacctgctagactgaagctttcctcccttttctgcctttagttttagtctcccgaacgcgcgagaagaatggacacccagaacccccagtactctcccttcctcgcagtgatgggtgcctcctcagcgatggtgttcagcgcgttgggtgctgcatacgggactgctaagagcggcacaggcattgctgccatgtcagtgatgcggccagagctgatcatgaagtccatcattcccgtggtcatggcgggtatcattgccatctacggcctggtggtggctgtactcattgccaacaatatcggtgataaaatctcgctctacaagagtatcctgcatctgggagcgggtctgagtgtgggtctgagcggtctcgctgccggcttcgccatcgggatcgtgggagacgcaggtgtgagaggaacggcccagcagccgcgtctgttcgtgggcatgatcctcatcttgatctttgcagaggtgctggggctctacggcctgatcgtcgccctcatcctgtccactaagggttaatcgttgctcctatcctctcttacacatgaacaaacacacacacagacacacacaccaacacaaatgatatacaacgagtaaacttcaccgcctgaataacgacgagaggaatttttgcgtgcatgcgtgtatcgtgagtgtgctgtttgcgatgagtgagcgtgagattgatcttcctctctgtgtaaagaagcccgatgtgtgtgtgtttttctttcccccctgtaaatgcgctgtgtagctatcgctctcgtcccgtgtgtgtgtgtgtgcagtctatacacctcaagctttgccagtggaacccatgtaccagcctgtggcacaatcaggctcagagtctatctctctcgctgaataccagatatcccctgttccaggacagattgtttcccagcctcaaatgcagcctggtagtctgtctctcactcagtccagctcttcttcaggcatgccattgcagtccagcttccagcctcttgtacaatcaagttatgagactgtcttacagcctggatttcaaactacttcagagcttgtgccatcaagtcaccagtctacaagttcccaagtcttgagtggtccagaagtttcaaaccatgagtctgtgtttcagccagtgcaacctgaatttgcaattccacaacaggttgaaaacaagttggttacaggtcaaaataatcctggacctgctcaaaatgggcattcatcgcagaagttgttcaggctgttgcaacaagtgaagtcttaggtgtttgctgtttcctgagtttgctttctgacttccaataaaatgtttaaaaag

SEQ ID NO: 20 - protein, 154 aa, Danio rerio
MDTQNPQYSPFLAVMGASSAMVFSALGAAYGTAKSGTGIAAMSVMRPELIMKSIIPVVMAGIIAIYGLVVAVLIANNIGDKISLYKSILHLGAGLSVGLSGLAAGFAIGIVGDAGVRGTAQQPRLFVGMILILIFAEVLGLYGLIVALILSTKG

SEQ ID NO: 21 - DNA, 3553 bp, Danio rerio
gccaggatattcatcagctgacccaagacagacatgcaaccaacacagctcggtttctcagatctgtcattcctttttcatttataaaaattcccttggcgcgtaattttgttttattttacaccaatagggtgtccattatgttttaatgagcacgcaaagaggacgatgacagtgacagcttgtgaacaggacatttaacgttagctcgcatgctagcgtgctatgggtggaaacagctggtagggatactgtacatctttacctgcaatgtaaacacaggttcactatttgctgtacatcgtcgtgtgaatatttgatcatttgcaatggcatctattctggatcaatatgaagattcgcagaacatccgtcaacacagtcgtatgtccacggccaacatcggcatcactcattcagggtttgtaaatgtaagattggaagaagaaaaacccattttcaccaagcagcgcatcgatttctctcctcccgaaaaaatcaaccaattttcggtttgcaacaatcagttatgcatgagcttgggaaaagacaccctgctgagaattgatctcggaaaacctgatcagccaaatcagattgaattgggaagaaaagatgatagcaaagtacacagacttttcctagatcctacagggtcacatttagtcatctgcctcactacaaatgaatgtgtgtacctaaatagaaacactcagaaggtccgaggtctgtcaaggtggagaggccacttgattgaaagcataggttggaacaaattaatcggctctgagaccaacactggccccattctggtcgggaccagccagggaatcatctttgaagctgagatctctgcctcagagggcagtctgttcaacaccaacccggaccagtacttcagacaggttcattacctcgaggaggatggcaagccagctccggtttgttgcctggaggttgaacgtggtttggaaactaaatactttatcattgcaaccacacgaaaacggctgttccagtttgtaggaaagctggcagaaggatctgagcagcagggcttcagctccatctttgcccagaaccaggatcttctacccagttccaggaatttccctgtcaatatgggctatagtgagattacattttacacatcaaagctgcgtagtcgccccaagaccttcgcctggatgatggggaatggcgttttgtacgggcaactggactatgtgcgccccgactctttactcagtgatgttcaagtgtgggaatacacgcaagacattgatttaaacttcgtcaaacccatctctatagtcctcacccagttccacttcttgctcctgctccctgaccgtgtccgagggatctgcaccctgaatgggcaggttgtgcatgaagatgtgtttcccgaaaagttcggtacactacaaaagatgatcaaagatcccataacaggacttgtgtggatctacactgagaaagcagtgtttcgatatcacatccagaaagaagcacgggacgtttggcagatgtacatgaacatgaataagtttgatctagccaaagagtactgcaaagatcgaccagaatgcttagacatggttctcgcaaaagaggctgagcactgctttcaaaacaagcgctacctagagagcgccaaatgctatgcgctcactcaaaattactttgaggagattgcgctgaagtttattgaggccaagcaggaggaggccttgaaagagtttctgattaagaagctggtcaatctcaagcccagtgagaagactcagatcaccctgctggtgacctggttgaccgaactctatctgaaccggctcggtcaactggaggccgacgaaggcaagcaacatctatttctggagacccgtgaagagtttcgtacttttcttaaaagccccaaacataaagattgcttctacaacaatcgcagcaccatctacgacctgctcgctagtcacggcgacgtggacaacatggtgtatttctcggtcatcatgcaagattacgagcgtgtcatttctcattactgccagcatgacgactacagcgcagctttagatgttctctccaaacactgtgacgacaagctcttctacaagttctccccagtcctcatgcagcacatccccaaaaaggtggtcgatgcatggattcagatgggaaaccggttggatccgaagaatctgatcccagcgcttgtgaactacagccagatgggcagcatgcagcaaataaacgagaccatccggtacatggagttctgtgtgtatgagctggacgtcaaagaagaggccattcacaattacctactctcgctttatgccaaacacaagccagacgctctgttgtggtacctcgaacaagccggaactcacgtctccgatattcattacgacttgaaatatgccttgcgcctttgctctgaacatggatacctgcaggcctgcgtcttggtttacaagattatggagctgtatgaagaagctgtggatcttgctctaaaagtggatgtggatttggccaagtcttgcgccgacctttcagaggacgatgaggagttaagaaagaagctgtggctgaagatcgctcgccatgtagtgcaggaggagaaggatgtcaagaaggctatgaactgcctgtccagttgcaacctgcttaagatcgaagacatcttgcctttctttccagactttgtcacaatagaccacttcaaggaggcgatatgcagctctttggaagagtacaacaaacacatcgaagagctgaaacaagaaatggaggaggccacagaaagtgccaaacgcatccgcgaagacatccaagagatgaggaacaagtatggcgtggttgagtctcaggaaaaatgcgctacttgtgatttccccctgctcaaccgacctttctatcttttcctgtgcggacacatgttccattatgattgcctcctgcaggaagtcattcctcacctctctgtctataagcagaataaattggatgagctacagaagaaactggcagcgaccacgcagaccaccaaggctcggcacaagcctagagaggaggacacggtcagcttaggcaaaggccagggaagcagggagcagatcaaatcagacattgatgatattatagcgtgcgagtgcgtgtactgcggtgagctgatgatcaagtccattgacaagccgttcatagatccgcagaagttcgatcaagagatgtccagctggctctgaaggagagacgcttctgcttgagctgcacaacacatttattatacacattgtacatttgcttaacatgtaaccggatgtgcattgtaaaatttttacattctcaattaatgaacatttatatgattttgtcaacaaatatgcactgtacaagtgttcttcgttgattttatgtgtttgcttccttagatcaaacttaaaaatgcacttgttttcattttgcataaaagtaatgcgaataatatcttttttatgtgcgcagctaagaagaataaatgattgccttttgactggaaaaaaaa

SEQ ID NO: 22 - protein, 974 aa, Danio rerio
MASILDQYEDSQNIRQHSRMSTANIGITHSGFVNVRLEEEKPIFTKQRIDFSPPEKINQFSVCNNQLCMSLGKDTLLRIDLGKPDQPNQIELGRKDDSKVHRLFLDPTGSHLVICLTTNECVYLNRNTQKVRGLSRWRGHLIESIGWNKLIGSETNTGPILVGTSQGIIFEAEISASEGSLFNTNPDQYFRQVHYLEEDGKPAPVCCLEVERGLETKYFIIATTRKRLFQFVGKLAEGSEQQGFSSIFAQNQDLLPSSRNFPVNMGYSEITFYTSKLRSRPKTFAWMMGNGVLYGQLDYVRPDSLLSDVQVWEYTQDIDLNFVKPISIVLTQFHFLLLLPDRVRGICTLNGQVVHEDVFPEKFGTLQKMIKDPITGLVWIYTEKAVFRYHIQKEARDVWQMYMNMNKFDLAKEYCKDRPECLDMVLAKEAEHCFQNKRYLESAKCYALTQNYFEEIALKFIEAKQEEALKEFLIKKLVNLKPSEKTQITLLVTWLTELYLNRLGQLEADEGKQHLFLETREEFRTFLKSPKHKDCFYNNRSTIYDLLASHGDVDNMVYFSVIMQDYERVISHYCQHDDYSAALDVLSKHCDDKLFYKFSPVLMQHIPKKVVDAWIQMGNRLDPKNLIPALVNYSQMGSMQQINETIRYMEFCVYELDVKEEAIHNYLLSLYAKHKPDALLWYLEQAGTHVSDIHYDLKYALRLCSEHGYLQACVLVYKIMELYEEAVDLALKVDVDLAKSCADLSEDDEELRKKLWLKIARHVVQEEKDVKKAMNCLSSCNLLKIEDILPFFPDFVTIDHFKEAICSSLEEYNKHIEELKQEMEEATESAKRIREDIQEMRNKYGVVESQEKCATCDFPLLNRPFYLFLCGHMFHYDCLLQEVIPHLSVYKQNKLDELQKKLAATTQTTKARHKPREEDTVSLGKGQGSREQIKSDIDDIIACECVYCGELMIKSIDKPFIDPQKFDQEMSSWL

SEQ ID NO: 23 - DNA, 3291 bp, Danio rerio
acgcgggatatcgttcagtccgacagggaagtgacctaactttctcggagcaggtgtctttggaggagctcacacttggaggcgtgaggaggggaaagatcacaataccgcatttccctctctagacctcctaaatatttagatgtttgcaaacatggtgtccaagttgacatctttgcaacaggagcttctgagcgccttgttggactctggagttaccaaagatgtgcttctgcaagctttggaggacctggacccgagcccgagcgcttttggagttaaactggacagtctgcagatgtccccgtccggctccaaactcagcgatacggattcaaagccggtgtttcatacgctcactaatggacacagtaaagggaagttatccggagatgaaggctccgaggacggggatgattatgacacaccgccaattctcaaagagcttcagtctcaaaacaccgaggaagcggccgagcagagggcagaaatagagcgaatgttggcggaggacccgtggcgcgcggcccgcatgatcaaaggctacatgcagcagcacaatatccctcagcgcgaggtggtggatgtgacgggcctgaatcagtcgcacctgtcgcagcacctgaataaaggcacgcctatgaaaacgcagaaacgcgcggcgctctacacctggtatgtgaggaaacagcgggagatcttgcgacaattcaaccaggccacacaaggctctggcgccaccatgttagacaaaggaaatcaggatcaggtactcctttttttctcagagtttagtcagtctgggcaaggtatggtccagccaggtgatgatgctgccattgagcctgcttgcaagaagctcagacgcaaccgtttcaaatgggggcctgcatcccaacaaatcctttaccaggcttatgagcggcagaagaaccccagcaaagaggagcgagaggcattggtggaggagtgcaaccgggctgagtgcctccaaagaggagtgtctccatcgaaagctcatggacttggctccaacctggtcacagaggtgcgagtctacaactggtttgctaacagaagaaaggaagaagccttcagacaaaagttggctatggatgcctacagtggcccagcacatagcctaaactccctcctttcacatagttccccgcatcacccacaaactagcagctccccaccaagcaagatgcaaggtgtccggtacagccaacaaggtccaggtgaagtcacttcttcaacaacgatcaatcaccatagcagcaatgccatgtcaaccagccagtcagtgttacagcaggtctctccaggggcgctggaccccagtcacagcctcctgtcacctgatgccaagatgatctcggtatcaggtggaggtcttcctccagtaagcaccctgaccaacatccacgcatcccaccacgtgcaccaacaaacccctaacctcataatgccactctctggagtcatggccattgcacaaagtttgaacacatcacaagcacagacggtgcctgtcataaacagtgttgcaggcagtctggcagcactgcagccagtacagttctctcagcagttaaatagccaacaccagcagctcatgcaacagtcatctggacacatgagccaacagtccttcatggcctctgtctcacactcacacatgtacccacacaagcaggagccaccccagtattctcattcgtctcggtttcccccggccatggtggtgacagacgccaacagcctcagtacactgagctcaatgtcctccagcaaacagtgtccactacaggcctggtgaatgtatacacctgacttcttcgatgccaagcaacaggagcacattgtcccaacctccattccccttcatcgcacctatatccatggcaacccataatatgtgcgagcggcacgctggatgatcagtgggtcaataaaccacgagctgaaggcaggactgatgagaatgaacactgttggggtgtttgggttcacagttgggtgactagaagtctggctcggatgatcaacatggcgcccaatttagtgacagtattttacttctcattgatggacaaactaaattaaggaacgcacatgatccatatgaacaaacaaatacgaaacgcatacaacacagtcagatgaagatgcaaagatggagcgggaagacctggaatccaggaataagagcacaaaatgtgtgtgtatgtaaaactttacaatcaaactgtgtggatccaatgacaatcagacaatgttagacgcatcgttaatgtaaaataaccgatgcaaagacgtcttccttgagatatcaatgcttagaaaatgttctgtacagttgggtgtaaatacgagcaattactatatcattattattattattattatttcattactatgaaggatgtaaatattcagttttatctgtctaaacatgtaaactgtgattgtgaatgtgacaatctaaggaacaggcatgatgtattggaatatgcagtgtgtcagagagaaggatttcaaaacttttcagaaagcttcgttgacgtcagatcttaaaagagagacaaatgtaaaagcacattcaacaaaacgttgaacctattgatccttctgtaagccttgttttaaaatagacactgttgtcaatgttgactgccattagtgtggcgtcagatagataagagtcatctttctgtgccagccaaagaaatactgaattaggaagtggtcgatcggagagactcagagataactctatctgtaaagaagttcacagtttttgtgatactgggaagaacggccatgaatcatgacatctctaatttaaatattgaccttcagtcaaactgtcattgtgatggtggggagatatctgaagaaaagagcgtcctgttgaattggtcagcgtaattgttatcagctggaatttcagaagttgaggaaatcgaatcagttcgccacttcaaacagtattagctgctcacgcaaccctgaactaccaaaaagcattgtgaaatagtttttactgtggcttttttttttacatttaggcccaatcccaattctaccccttagcccttttccttactcctaaccctcgttttgcgtgttcatgtgaaggggtaaaggtgtccaaattttttttggcttgaaggcatagggctaatgggaagggctaaataccccctgaaacagatttttaaggaccacacttgaaaccaaggggtaagaaaatttcccaaaaaaaaaaaaaaaaaaaaaaaaaaa

SEQ ID NO: 24 - protein, 559 aa, Danio rerio
MFANMVSKLTSLQQELLSALLDSGVTKDVLLQALEDLDPSPSAFGVKLDSLQMSPSGSKLSDTDSKPVFHTLTNGHSKGKLSGDEGSEDGDDYDTPPILKELQSQNTEEAAEQRAEIERMLAEDPWRAARMIKGYMQQHNIPQREVVDVTGLNQSHLSQHLNKGTPMKTQKRAALYTWYVRKQREILRQFNQATQGSGATMLDKGNQDQVLLFFSEFSQSGQGMVQPGDDAAIEPACKKLRRNRFKWGPASQQILYQAYERQKNPSKEEREALVEECNRAECLQRGVSPSKAHGLGSNLVTEVRVYNWFANRRKEEAFRQKLAMDAYSGPAHSLNSLLSHSSPHHPQTSSSPPSKMQGVRYSQQGPGEVTSSTTINHHSSNAMSTSQSVLQQVSPGALDPSHSLLSPDAKMISVSGGGLPPVSTLTNIHASHHVHQQTPNLIMPLSGVMAIAQSLNTSQAQTVPVINSVAGSLAALQPVQFSQQLNSQHQQLMQQSSGHMSQQSFMASVSHSHMYPHKQEPPQYSHSSRFPPAMVVTDANSLSTLSSMSSSKQCPLQAW

SEQ ID NO: 25 - DNA, 474 bp, Danio rerio
ccacgcgtccgcccacgcgtccggaaaaatggcaaagatcaaggccagagaccttcgcggaaagaaaaaggaggagctgctaaaacagctggatgatctgaaggtggaactttcccagctccgcgttgccaaggttaccggtggagctgcttccaagctctcaaaaatccgtgttgtccgcaagtccatcgccagagtcctcacagtcatcaaccagacacagaaggagaacttgaggaagttttacaagggtaagaagtacaagcctttggacctgagacccaagaagacccgtgccatccgtcgtcagctcacaaaacacgaagagaacctgatgactaagaagatgcagaggaaatctcgtctctactccattcgcaagttcgccgtcaaggcataaaaaagctgtacttttgtttttcactgaaaaataaaatcccccgctcaccctggaaaaaaaaaaaaaaaaaaaaa

SEQ ID NO: 26 - protein, 123 aa, Danio rerio
MAKIKARDLRGKKKEELLKQLDDLKVELSQLRVAKVTGGAASKLSKIRVVRKSIARVLTVINQTQKENLRKFYKGKKYKPLDLRPKKTRAIRRQLTKHEENLMTKKMQRKSRLYSIRKFAVKA

SEQ ID NO: 27 - DNA, 388 bp, Danio rerio
cgagggcaaccatggtaaacgtaccgaagacccgcaggacgtactgcaaaaaatgcaagaaacaccagccgcataaagtgacccagtacaagaagggtaaagactctctgtacgcccagggaaagaggcgttacgacagaaagcagagtggttatggaggacagaccaagcctattttccgaaaaaaggctaaaaccacaaagaagatcgtgctgaggctggagtgcgtggagcctaactgccgctcaaagaggatgctggccattaagagatgcaaacactttgagctgggaggagacaagaagagaaagggccaggtcatccagttttaagctgggccaagtgcttcttttgtggattaccatcgctgtcaataaaagttgtatac

SEQ ID NO: 28 - protein, 106 aa, Danio rerio
MVNVPKTRRTYCKKCKKHQPHKVTQYKKGKDSLYAQGKRRYDRKQSGYGGQTKPIFRKKAKTTKKIVLRLECVEPNCRSKRMLAIKRCKHFELGGDKKRKGQVIQF

SEQ ID NO: 29 - DNA, 1025 bp, Danio rerio
cgtaagcggatgtacgaatacgcaagaattcaacgcacgaaggaagagcgagcgacatctgtacagtacaagatggatacgctaatattggagccttctctgtacactgtaaaagcggttctgatcatggataatgatggagagagactgtatgcaaagtattatgatgacacgtatcccacagtgaaagagcagaaagcctttgagaagaacatctttaacaagacgcacagaacagacagtgaaatcgcgttgctggagggtctaacggttgtgtacaagagcaatattgatttatatttctatgtaattggcagctctcatgaaaatgagctgatgcttatgtcagtgttgaattgtctctttgattctctgagccaaatgctgaggaaaaatgtggaaaagagagccctgctggaaaatatggagggtcttttcttggccgttgatgagattgttgatggaggagtgattctggagagcgacccacagcaggtggttcaccgtgtcgcattgaggggcgatgacgtgcctctgacagaacagacagtcactcaggtgctacagtcagcgaaggagcagatcaaatggtctctcctgcgataggattcttcagctttctgtcctgaactgaagcgaggaaaagcaatgagagagcacctcttttcatcactactgtccctttctgcccttttctccgtgagtgttttcatgactgttaaagtgcacatccactgaaaaccacttctccagtttctctcactccagataagaccagcaacagcgctatttctacacattaacacaagaggaagaagcttgccgggactttctttttgggaaaagtaccggggcgtttgtttttttttaaagcccattccagtaagcgcttgaatatatggctggtttagtgaatgatatcactgttgtactgtgagattttcatgaggattcatgtacattccagtggtcgatcttttctctgtttttggattgaagtaaagagtttcatcccaaaaaaaaaa

SEQ ID NO: 30 - protein, 177 aa, Danio rerio
MDTLILEPSLYTVKAVLIMDNDGERLYAKYYDDTYPTVKEQKAFEKNIFNKTHRTDSEIALLEGLTVVYKSNIDLYFYVIGSSHENELMLMSVLNCLFDSLSQMLRKNVEKRALLENMEGLFLAVDEIVDGGVILESDPQQVVHRVALRGDDVPLTEQTVTQVLQSAKEQIKWSLLR

SEQ ID NO: 31 - DNA, 2144 bp, Danio rerio
gactctccccatgtgtttacttttgtggaataaatttatttaaactatggaaaacattaccgatgggggatgggggagtctgccggtgaaattacatgacaacatattacagactcttaaagagctgggatttacatatatgactcccgttcagtctgcctgtattcctctttttatgagtaataaagatgtggctgccgaggcggtgactggcagtgggaagactcttgcctttgtgatccctgcattagaaattcttctaaagcgagaagagaagttaaaaaagatgcaggtcggtgcgttgatcataacgcccacccgtgagctggctatgcaaatcagcgaagtgatgggtcgttttctgcagggattccctcagtttacacagatccttttaattggtggaagcaaccctattgaggacgtggagaagttgaagactcagggagccaacatcataattgcgactcccggccgattggaggacatgtttaggaggaaggctgatggacttgatttggccacggctgtgaagtctctagatgtcctagtcctggatgaagctgacagattgttggacatgggctttgaagccagtttaaacaccattctggggtatttacccaagcagcggcgcacagggcttttctccgccactcagacgcaggagctggagaagctggtgagagccggtctcagaaaccctgtgcgaatcaccgtgaaggagaaaggggtcgcggcctccagtgtgcagaaaacccctgccaaactcagcaactactacaccatgtgtcgagcagaggagaagttcaacactttggtggcctttctcaggcagcacaagcatgagaaacagctggttttcttcagcacctgtgcctgtgtggaatatttcggaaaagccctggaagtcctggtcaagaacgtcagcatccactgcattcatggaaagatgaagcacaaacgcaacaagatttttgccgatttcagggctctgaagagtgggattctggtgtgcacagacgtcatggcgagaggcatcgacatacctgaagtcaactgggtgcttcagtatgacccgccaagcagtgcaagttcctttgtgcatcgctgtggacgaactgcacgcattggaaaccaaggaaatgcgcttgtctttcttctgccaatggaagagtcatatgtcaatttcttgtccattaatcaaaagtgtccccttcagtcgttttcctcagtgaaagatgtggttgatgttctgccgaaattgaaggcgatggctttaggcgaccgagcgatgtttgagaagggcatgagagcctttgtgtcgtatgtgcaggcgtacgccaaacatgagtgcagtctgatatttcgcattaaagacctagatttcgccgctctggctcgtggctttgctcttctccgattacccaaaatgcctgaattaagaggaaagaccttcccagacttcaaagcggaggccattgacacagacaccatccgctttaaagataaaaacagggagaaacagagacaaaagtggctggcggagcagaaagagaaagaggtgccgctgaggaagaacttcatcaagaacaaagcctggtccaagcagaagatcaagaaggataggaagaagaagaggcttccaaaagcaaagctagatcaggattcggacgctgcagaggaagacttaaacgagcttatgaatgacacccgtctgctgaagaaactgaagaaagggaaaatcaccgaggaggacttcgacaagcaaatgagttcaacggacaaacacaaaccagcaggaatagacagttctgatggagactgagaaagagacgtcatgtgtgagacttcatgctgtcgtttcaattcgatatgctgtatcagcagggttacttcctttctgattgactgtctgtgtgatgaacgaagtccatgcgttcatcaccaatgtgtttttgtactaacaatattatacacagtacgtcacttggaattaaaaatgtataatgctgtcaaatcatctctgcaggactgggggatttatactttttcattaaaaataatgttgagatgaattgcgctgaaaataaaccaaatggaagatgactcccaccccccaaaaaaaaaaaaaaaaaaaaaaa

SEQ ID NO: 32 - protein, 593 aa, Danio rerio
MENITDGGWGSLPVKLHDNILQTLKELGFTYMTPVQSACIPLFMSNKDVAAEAVTGSGKTLAFVIPALEILLKREEKLKKMQVGALIITPTRELAMQISEVMGRFLQGFPQFTQILLIGGSNPIEDVEKLKTQGANIIIATPGRLEDMFRRKADGLDLATAVKSLDVLVLDEADRLLDMGFEASLNTILGYLPKQRRTGLFSATQTQELEKLVRAGLRNPVRITVKEKGVAASSVQKTPAKLSNYYTMCRAEEKFNTLVAFLRQHKHEKQLVFFSTCACVEYFGKALEVLVKNVSIHCIHGKMKHKRNKIFADFRALKSGILVCTDVMARGIDIPEVNWVLQYDPPSSASSFVHRCGRTARIGNQGNALVFLLPMEESYVNFLSINQKCPLQSFSSVKDVVDVLPKLKAMALGDRAMFEKGMRAFVSYVQAYAKHECSLIFRIKDLDFAALARGFALLRLPKMPELRGKTFPDFKAEAIDTDTIRFKDKNREKQRQKWLAEQKEKEVPLRKNFIKNKAWSKQKIKKDRKKKRLPKAKLDQDSDAAEEDLNELMNDTRLLKKLKKGKITEEDFDKQMSSTDKHKPAGIDSSDGD

SEQ ID NO: 33 - DNA, 935 bp, Danio rerio
tgcacttcattaagtgttggcgcagagacgcgtctgtaaaccaataaagcgacgaagaggacgttattcatctcgggctttaaattaacgtcaccaccgagagtgacatgcagtctttgtacccgtaaatgattacaaactaacagagcgaactaaaaatcaagatagagtcacaggatgtactttactttaggtaacgttaacgttagcggtatctattgtcattctccccaagctgcagtcgtgctcgtcgtaatgggtttccatgggcggagtgtgtaaaaaccaagggtctccttcagtctcggctccagcatcagcagcaggtcatcatgaggatgagactgaagctgaagacagtgtttgtcctgtacttcatggtgtctctgtttgggctcctgtacgcgctgatgcagctcggtcagcgctgtgactgtcgagatcatgagcaatctaaagatcagcagatctctcagctgaaaggggaactgcagaagctgcaggagcacatcaaaacatcagagctgtccaagaagacggatgtgcccagaatatacgtcataacacccacatatgctagactggtgcagaaagctgagctcactcgtctatctcacacattcctccacgttccccagctacactggatcgtggtggaagacgctcctcagcaaactcagctcgtctccgacttcctgtctgcttccggcttgacctacactcatctaaacaagctgacgcccaaggagaggaagctgcaggagggagaccccaactggctgaagccccggggggctgagcagaggaacgagggtctgcgctggctcagatggatgggttctacagttcacgggaaagaagcagctgcccttgaagaggccgtggtttactttgctgatgatgacaacacgtacagcctacagctttttgaagag

SEQ ID NO: 34 - protein, 201 aa, Danio rerio
MRMRLKLKTVFVLYFMVSLFGLLYALMQLGQRCDCRDHEQSKDQQISQLKGELQKLQEHIKTSELSKKTDVPRIYVITPTYARLVQKAELTRLSHTFLHVPQLHWIVVEDAPQQTQLVSDFLSASGLTYTHLNKLTPKERKLQEGDPNWLKPRGAEQRNEGLRWLRWMGSTVHGKEAAALEEAVVYFADDDNTYSLQLFEE

SEQ ID NO: 35 - DNA, 874 bp, Danio rerio
attgtattcactctcgctaaacttaacaatgattggtcagtgtaataatgagatcgcagagtgtgattaatgtaatcgctgtgtttccgtcagtaatgcccggatcagcgttgttatcgctgcagcaggagctccggcggggattcgaggctctaaaggccgccaaagatacgtttgatggcggtcatgcggagtgtcaggagctcgtctcctctctggggaacctggcgctccagctgaaggctctcaaacaggtccagatatccgaaactcctctctccggattcccatgtttacagcagcgactccattacaagctctcgctggcggtggacgcacttctggctaaactagccgagaaaatggctgctcttcagaaggtgcgggatgccttcgctcagcaggtgtttactgtcgtgcagctctatgagaaaaacacagacgctctggacatcagcgtgtgcgtctccagatcagccgtctctccttcagtctcagacatgctggagtggcttcaggacgccgaccgatattaccgcctgcaattaatgcaaagccggctcctccttcagacgctgacccccactggccttacatcaatggaaacagcaccaaatagatggagatctgtgcgttctgcccgtcaggaggagagaattgcagatgcattgtgtcaagtgtcgatcttcttggagactgaatgaatgaaggatccttaaaaagagacagactgaactctgtctcctggattaatgtagttttatggtgcgatttagcattataaggactgtgcatcagcatcactgtaagagcaatgctctactgtaataaatgcaattcatgctttttatattcaaaaaaaaaaaaaaaaaaa

SEQ ID NO: 36 - protein, 218 aa, Danio rerio
MRSQSVINVIAVFPSVMPGSALLSLQQELRRGFEALKAAKDTFDGGHAECQELVSSLGNLALQLKALKQVQISETPLSGFPCLQQRLHYKLSLAVDALLAKLAEKMAALQKVRDAFAQQVFTVVQLYEKNTDALDISVCVSRSAVSPSVSDMLEWLQDADRYYRLQLMQSRLLLQTLTPTGLTSMETAPNRWRSVRSARQEERIADALCQVSIFLETE

SEQ ID NO: 37 - DNA, 1050 bp, Danio rerio
caacccacagaaggcggggggtggttttcaagatggcgccgctttcttcgtccacacgtgaggtgttcagtgaagcagttcgggctgttttggaaacttggccggtgctgcaaatagcggtggacaacggcttcggtggagcgttcagtcagcagaaggcggagtggatggtggacgcgctgcagcaatacttcaccgacaacgatgatctccagccggatgaggtggaggacttcatctctgagctcatgaacaatgagttcgacacagtggtggatgatggcagtctgcctcaggtggcgcagcaggtgtgtgggatgttccagcagtgtgagcagggcagattgttggaggtcagagagcagatccttaaactgaaccagaagaagacaagcagcggaagagctaaagccactcctgcacaaacacctcaggatgatgatgatgatgatgatgaggaagaggctatggattgtgaaggaggatcagcgggggcttcagtcagcagtacagcagtgaatgatcttcatcatgaggaggaggaagatgacggatggacagtcgtgcgcagaaaaaagtgaccaatctctcacttcctgcattcatgacatttaaagacatttgaagggcttgtgtcacttctactttggacttttctgctttttcagccactgatataaaatggtaggatctcctcaggcaaccttatgaagtcctgtctctggagcacagattggatgttgtccagggaaattcaggtgcatatacagtcatgcatttaaatgtttatggtcgagtcatttatcacagtttctctgtttgacttcatctgaatgcatttatttataagcggtaggtcaatgcctgctcctctgtcgcccccttgtggaactatagaatccagcagcagtggtgaatatgctgtttaacaggcacactaagagaaaaaactgtagttacagagcttgttaaatgattaacatcataatttcttccacattttaaataaaaatattgtctgttttcaaaaaaaaaaaaaaaaaaaaaaaa

SEQ ID NO: 38 - protein, 182 aa, Danio rerio
MAPLSSSTREVFSEAVRAVLETWPVLQIAVDNGFGGAFSQQKAEWMVDALQQYFTDNDDLQPDEVEDFISELMNNEFDTVVDDGSLPQVAQQVCGMFQQCEQGRLLEVREQILKLNQKKTSSGRAKATPAQTPQDDDDDDDEEEAMDCEGGSAGASVSSTAVNDLHHEEEEDDGWTVVRRKK

SEQ ID NO: 39 - DNA, 2999 bp, Danio rerio
aaacttaatgcacaaaatctccatctgataaaacaaataaagtgttaaatccgagagcagtctatcgggttacattttttgcagtctaataataaacaatgtcgctttcagcagcgcgttcgtttctttcggaggcggcatacggcgagcaggagctggacgccaattcagcgctgatggagctcgataaaggtttacggtcttgtaaactgggcgagcagtgtgaagccgtggtcctttttcccaaactcttccagaaatacccgttccccatcctcatcaactctgccttcctgaagctggcagatatcttcagacttggcaacaatttcctgcggctgtgtgtgctgaaggtcacgcagctgagtgaaaaacacttggagaagatcctgaatgtggatgagtttgtcaaaagagtgttttctgtcatccacagcaatgaccctgtggcccgagcaatcaccctcagaatgctgggaagtttagcttccatcattccagagagaaagaacgctcaccacagcatccgccaaagcctggactctcatgataatgttgaagtagaagctgctatttttgcagccgctagcttctcatcacactccaaagattttgcagcaggaatttgcaacaagattagtgagatgattcaaggactggacactccagtagagctaaagctgaagttgatcccgatgctgcagcacatgcaccatgatgccagcctggcgtcgtgcagcagagagttactgcaggagctggtgtcctcgtacccatccacatccatgctgatcgtcacactgcacacctttacccagctggccacatcttcactcgttgacataccggagcagatttgtcttcttctgcagtacttgaaggaagaccccagaaaagctgtaaaaagactctccattcaggatctcaagctcctggccaaaaaggctcctcatttgtggaccaggaaaaacatacaggtgctgtgcgagtgtgcactccacaccccatataacagcctgaagctgggaatgctgtctgtcctctccacattgtctggcaccatcgctattaaacagtacttcagccctaatgcaggtgattcatctcctgcacctcaccacactgacctggtaaagctggcacaggagtgctgttatcacagtgacctggcagttgcagcacatggaatcacagtgttgactagcattgctgctttctgccctgagaaagaggtcattcagctggaacaggaaacagtgatggggatggagtctctcattctgctctgcagtcaagatgacagtaagactgcccaggccacactaaaaacggcgctcacgtctctggtgcagatgttgaagacttgtcctcatctcagtcagagctcagtggagctcctgcttaggcagctgcattgcgcatgtgaccctgctcgggtgctcatgtgtcaggctctggctgctattgccacacaacagccagtgctggtagagggaatgctgggagacctgctggagctcttcagggtggcaagtcacaggacttcagagaaacagcaggagcttctggtgtctttggcaactgtgttatttgtggccagtcaggcgtctctctcagctgaggtgaaggctgtgatcagacaacagttggagaatgtggccaatggctggaccgtgtaccaaattgccagacaagcgtcccgcatgggctgccatgatttttccagggagttgtaccagagtcttcgcacccgtgtggcctccgaacacttttacttctggcttaacagtctgatggagttttcccaagcggagcagtgccttagtggcctggaagatggagactacagtgcagccatgagcgccatctctgaagcactgaaatcctaccagaagggaatcgcatctctcacggctgccagtacgccgctcagtccgctgacattccaatgtgagtttgtgaagctgcgtatagacactcttcaggctctgtctcagctcatttgcacctgcaacagtctgaagaccagtcctcctcctgccatcgcaaccacgatcgctctgtcctcgggcagtgacttgcagcgctgcggacgaatctccacacagatgaagttttccatggatgagtttagaagtctcgcagctcgctatgctgacctgtaccagtcttcgtttgatgcggactatgccacccttcgcaatgttgaactacaacagcagagttgcttactcgtatcttatgttatcgaagctttaataatcgatccacaaacagccagtttccaggagtttggcactcatggatcaattctggcagagagcgagtatgagctgagaatgatggctgtgttcaatcatgtcctggaggaagtggaaaatctaagcaggaaacaccctcctgtttcttacctgcacaccggctgtctgtgtgatactgtcatagccatcctgaaaataccactgtcgttccagagatatttctttcagaagcttcagtccaccagcattaaacttgctctctctccatctccccgcacacccaatgagccgatcccagtgcagaacaaccagcagctgaccttaaaagtggagggtgttattcagcacggctccagtccgggactcttccggaagattcaggcagtctgtcttaaagtcagctcgacgttacagacaaaaccaggatcagacttcaagattcctctggaatccaaaaccaacgagattgaacagaaagtagagcctcataacgattacttcagcacacagttcctgctcaacttctccatcctgggcacacaccaggtatctgttgaagcctcagtggtggacaccagtgggattgaatggaagaccggccctaaaaacactgtttcggtgaagtctctggaagacccatattcccagcagcttcggcatcaattacagcagcagcagcagaacgtcccgcagcctgcagcccagaggaacatcagcacacgcttccaataaaaacac

SEQ ID NO: 40 - protein, 965 aa, Danio rerio
MSLSAARSFLSEAAYGEQELDANSALMELDKGLRSCKLGEQCEAVVLFPKLFQKYPFPILINSAFLKLADIFRLGNNFLRLCVLKVTQLSEKHLEKILNVDEFVKRVFSVIHSNDPVARAITLRMLGSLASIIPERKNAHHSIRQSLDSHDNVEVEAAIFAAASFSSHSKDFAAGICNKISEMIQGLDTPVELKLKLIPMLQHMHHDASLASCSRELLQELVSSYPSTSMLIVTLHTFTQLATSSLVDIPEQICLLLQYLKEDPRKAVKRLSIQDLKLLAKKAPHLWTRKNIQVLCECALHTPYNSLKLGMLSVLSTLSGTIAIKQYFSPNAGDSSPAPHHTDLVKLAQECCYHSDLAVAAHGITVLTSIAAFCPEKEVIQLEQETVMGMESLILLCSQDDSKTAQATLKTALTSLVQMLKTCPHLSQSSVELLLRQLHCACDPARVLMCQALAAIATQQPVLVEGMLGDLLELFRVASHRTSEKQQELLVSLATVLFVASQASLSAEVKAVIRQQLENVANGWTVYQIARQASRMGCHDFSRELYQSLRTRVASEHFYFWLNSLMEFSQAEQCLSGLEDGDYSAAMSAISEALKSYQKGIASLTAASTPLSPLTFQCEFVKLRIDTLQALSQLICTCNSLKTSPPPAIATTIALSSGSDLQRCGRISTQMKFSMDEFRSLAARYADLYQSSFDADYATLRNVELQQQSCLLVSYVIEALIIDPQTASFQEFGTHGSILAESEYELRMMAVFNHVLEEVENLSRKHPPVSYLHTGCLCDTVIAILKIPLSFQRYFFQKLQSTSIKLALSPSPRTPNEPIPVQNNQQLTLKVEGVIQHGSSPGLFRKIQAVCLKVSSTLQTKPGSDFKIPLESKTNEIEQKVEPHNDYFSTQFLLNFSILGTHQVSVEASVVDTSGIEWKTGPKNTVSVKSLEDPYSQQLRHQLQQQQQNVPQPAAQRNISTRFQZ

SEQ ID NO: 41 - DNA, 2348 bp, Danio rerio
gttcatcccgagacggacggatggcgaagtgagtgggttgcgctttgaagagaaagcagcgactcacaaaaattcttcaacgttaaaacgtcggttgccttgatgttcgttcaaataagcgggaaaacgcatctttaagaagaaaaatcgcccctcagagcgaggtgaatcgaagagagcgtcgaggacatcgactcctccgtgtcctttcaggctaagctgctagtcagctattcggggaattgctcaagagaaaaaacagcctcaacctcaggggaaatagtcttgttttttttttttttacttaaatgtcgaggcagcaaaacttatattgaaaccgatgtccgttatcatcttcatttcgtcgttgtgagttaaggacacagctagtaaggaagagccgtttttaggctagctagcattgtttctactggactgaagaagagagctgcctgaaaacctcgcccgtttaccagtttcagtgtacttctcggattttaggacatggccagcagcagtggctctaaagccgagtttatagtcggtgggaaatacaagctcgttcgtaaaatcggatctggatccttcggtgacatttatttggcaatcaacatcacaaatggagaggaggtagctgtgaaattggaatcacagaaagccagacatcctcaactcctatatgaaagcaaattgtacaaaattctccagggaggagtcgggatcccacacatcaggtggtatggccaagaaaaggactataatgtcctagtcatggacctgctcggcccaagtctggaagatctctttaacttctgttcccgcagattcacaatgaaaactgtcctaatgcttgcagatcagatgatcagcagaatcgagtatgtgcacacaaaaaacttcatccacagagatatcaagccagacaactttttaatgggtattggccgtcactgtaataagttgttcctcatcgattttggtctggccaagaaatacagagacaacaggacacgacagcacataccctacagagaagacaaaaacctcacaggcacagctcgctatgccagcatcaacgcacacttgggcatcgagcagagtcgtcgagatgacatggagtcgctaggatatgtcctgatgtacttcaacagaaccagcctgccttggcagggactgaaggctgccacaaagaaacagaaatatgagaagattagtgagaaaaagatgtcgacccctgttgaggtgttgtgtaagggtttcccagcagagtttgccatgtacctgaactactgtcgtgggttgcggtttgaagaggcgcctgactacatgtacctgcgtcagcttttccgcattcttttcaggactctgaaccaccagtatgactacacatttgactggaccatgctgaagcagaaagcagcacagcaagcggcctcctcagggggacaggggcaacaggcacaaacccccacaggtttctaagcattaagacaatgtaatgaagcagagcagagtggtcgtagcaggatttgccttccaatcacaccccggcatctaggatcaaattcaccagaacctgcaggcactgtgggcaacctttttcctgttaaagaactttcgttccatatactatatatgtaattggtatatatagataagatgctccgcatatatctatatattggtatgtatatactatatccacacacagaagcaggcttggtttggagttggaagctgtagccttttctattgtcctgtcccttaagacttttcgccccctccttctttgaaacggaaaactgtagaaaatggggatgtaattgcgcacatgccaatgtttttcccgagttttctccatcaggaaatctgtttggggtccttcctgaattcttggttgcccatcagagcagttttggcccccttttcccctttgtcatcgtcacagagtgtggcacgaacccagacactcttcagaatggagacatccgagacacattttcatcctttaatttttttattcttcttcctttcttcaaacaaagtgaaacatctgagattaaacctgagtttgtttttcgttctttctttttcttttggttttgttccaagtgtatccaaataattaaaaaggcaaacattgggttttgttctttttttctaaaaaggtaattacgattctgcctgctgatatgtgctagagagcttctaatgtgaagattaaaaacattctagtgactactaggtttctctttagagcgctgcattaactatattgtacttggtattctgaagtctaaaaaaaataaaataaataaaagagtaaactcccaccaaaaaaaaaaaaa

SEQ ID NO: 42 - protein, 325 aa, Danio rerio
MASSSGSKAEFIVGGKYKLVRKIGSGSFGDIYLAINITNGEEVAVKLESQKARHPQLLYESKLYKILQGGVGIPHIRWYGQEKDYNVLVMDLLGPSLEDLFNFCSRRFTMKTVLMLADQMISRIEYVHTKNFIHRDIKPDNFLMGIGRHCNKLFLIDFGLAKKYRDNRTRQHIPYREDKNLTGTARYASINAHLGIEQSRRDDMESLGYVLMYFNRTSLPWQGLKAATKKQKYEKISEKKMSTPVEVLCKGFPAEFAMYLNYCRGLRFEEAPDYMYLRQLFRILFRTLNHQYDYTFDWTMLKQKAAQQAASSGGQGQQAQTPTGF

SEQ ID NO: 43 - DNA, 1400 bp, Danio rerio
gccacttcaaagacccaaaacgacctcactgaccatcaaaagactgcaagtttctataagtgaacttagatgaccggccagcactcatgacattcactttccagaggacggtgtaagagaaagagctcagagactttatttcaataactgcgtgtggattattaccttgatttgacatgttttcctgcgggctcctgagcgtagttttggccctcgcggtcggcctggtgagctgcacgggaaaccttgctggatttcaagagaccctcaggaataaaatgcgagcagctggcagaaacggcggaggtcgggcaggtcacggccgacatctgaccagatatccgctgtatatgatgcacctctaccggacacttctgactggagacgaaaaacacttcagccatgagaatccaactctttacgagtctgactccgtcttgagcctcgtcgctaaaagttgtcatcaggttggtgacaaattcgcagtgacatttgacatgtcctccatatcagcaagcgatgacgtccagcgagctgaacttcgtattcggcttccgcatctcaggtctgagttggaggtggatatttatcacgcatctacaccggagtgtgagagaagcccctgcgaagaagtccgggtccacctgggaaccttgaacgccaacccgattaactcaaccttccgctcctcttggaggatcttcaacatcactgcgctcctcaagtactggttgcaccagagtgagcgtgtgccgtttgaggaacccacacagatgccgccaatggctgagggacacaagagcgttcatcatcctacagcgaaccgagtgatgatggtggtttactccaagcagaaccgggcaaagacgtccactctcatccggactgccgagcactccaagtatgtagctctggatcgggctggtggtggaagtgaacctgtgcctcggcgccacagaaggaaccacagaactgatgatagggtgcgcgatgcagcagcagggatgattcctggtgtttcccatgaaggtggagagaagaaacctctctgcaagaaggtggatatgtgggtggactttgatcagattggttggagcgactggattgtttatccaaagcgttacaacgcttatcggtgtgaaggcagttgtcccacaccagtagatgaaaccttcactccaacaaaccatgcatacatgcagagtcttttgaagctgcaccacccagatcgcgtcccatgcttgtcctgcgtacccacccgtctggctccactctccatgctctactatgagaatggcaagatggtcatgagacaccatgaaggcatggttgttgcagaatgcggctgccactgattcttcaaaccccaaaggaactcaactctagcactttggatatgc

SEQ ID NO: 44 - protein, 392 aa, Danio rerio
MFSCGLLSVVLALAVGLVSCTGNLAGFQETLRNKMRAAGRNGGGRAGHGRHLTRYPLYMMHLYRTLLTGDEKHFSHENPTLYESDSVLSLVAKSCHQVGDKFAVTFDMSSISASDDVQRAELRIRLPHLRSELEVDIYHASTPECERSPCEEVRVHLGTLNANPINSTFRSSWRIFNITALLKYWLHQSERVPFEEPTQMPPMAEGHKSVHHPTANRVMMVVYSKQNRAKTSTLIRTAEHSKYVALDRAGGGSEPVPRRHRRNHRTDDRVRDAAAGMIPGVSHEGGEKKPLCKKVDMWVDFDQIGWSDWIVYPKRYNAYRCEGSCPTPVDETFTPTNHAYMQSLLKLHHPDRVPCLSCVPTRLAPLSMLYYENGKMVMRHHEGMVVAECGCH

SEQ ID NO: 45 - DNA, 3045 bp, Danio rerio
caacacattttcctccatcccaaaagtgggggatggtgaacgctatttggtttgttctaccttttcctgtaagaaactggaaggaagcaaatttttgccgttgaacttacaaaaagaaactattgttgacaactttttgcgcaccgcaattgtacgatgaacagattttcaacagtatgtgcccgagaacggaggcataaaaaactttggaatcggactaaaaggtgtttcttttaataggaaattttgtttaatcgcgattctatgtcgtatccaaaatttgtcgtgttttgaaaactcaacgcgcatcgcgcacgagtggaactcgcaagtttatttggaaacatttgatgaaacatcgctactttgttgcgtctccaagatgtcctccaagcgcccctgctccattgttggaagcttttggatgctttggatctggacagctacctctatggtcgccagggcggtcatcttgcacccaaatgaaactatctttaacgacttttgtaagaaatctacaacttgtgaagtgctaaaatacaatacgtgcctaggctcgcctttgccgtacacgcacacgtctctgattctggctgaagactcagaaactcaagaggaagccttcgagaaattggccatgtggtctgggttgagaaatgctcctcgctgttgggccgtcattcagccgttgctgtgtgccgtttacatgccaaagtgtgagaatggaaaggtggagctgcccagccagcacctgtgccaagctacacgcaatccatgcagtattgtggagcgggagaggggctggcccaacttcctaaagtgtgaaaacaaggagcagttccctaagggttgccagaatgaagtacagaagctcaaattcaacacttcaggccaatgcgaggctccgttggtcaaaaccgacatccaggcaagttggtataaggatgtggaaggctgtgggatccagtgcgacaaccctctgttcacagaggacgagcactcagacatgcatagctacatcgccgtcttcggtaccatcactctcctctgtaccttcttacacctggccacatttcttgcagactggaagaattccaaccgttaccccgctgtgatcctcttttacgtcaacgcctgtttcttcattggaagtattggatggctcgctcaattcatggatggagcccgcaatgagatagtatgcaaaagcgacaacactatgcgacttggagagccatcatccaccgagacgctgtcatgcgtgattatcttcgtcattgtgtattactcgcttatgtctggagttatttggttcgttatgctcacttatgcctggcacacatcattcaaagcccttggcaccacccatcagcctctgtctggaaaaacgtcctatttccacttggtcacgtggtctattccttttatactgaccgttgctatactggccaattcacaggtggatgcagattcagttagtggcatttgctttgtcggctataggtattatgaatatcgtgccgggtttgttctggctcctattgggtttgtccttgttattggtggctatttcttgatacgaggggttatgacattgttttccattaaaagcaaccatccagggctgctcagcgaaaaagctgccagtaaaatcaatgaaaccatgctaaggcttggcatatttggattcctggcttttggttttgtcctgattacattcggatgtcatttttatgacttcttcaatcaagctgagtgggaaagaagcttcagggaatatgttctatgtgaagctaatgtcaccatagctcaccagaccaacaagccgatcccagaatgtgccatcaagaacaggcccagtctgctggtggggaagatcaacctcttctcaatgtttggcacaggcattgcaatgagcacctgggtttggacaaaggcaacaatcctcatttggaaacggacctggttcagaatcattggacgaagcgacgatgagccgaaacgcattaaaaagagtaaaatgattgccaaggcattctcaaagcgaaaggaactccagaaagatcctgagaaagagctgtcctttagcatgcacacagtttcccatgagggacctgtggcgggtatcaactttgacctgaatgaaccgtcgattgagatgtcttctgcatgggcccaacatgtgaccaagatggtggcaagaagaggggcaattttaccgcaggatatctcagtgacacccactggaacacccattccgcctccagaggaaaggaacaaactttggatggtcgaagcagaaatttctcctgaaatgatgaaaagaaagaagaagaagaaaaagcgaaggaaggaagtacggccagcgggtcctgcagcagatgaagggaatcctgcctaccaccggagggagtttggccccagtgcagtgccgcgtctcccaaaacttcctggtcaccggagtctggtggctaacctttgggaacagcagcgacagcagcaggaggaacaggatatgctacctggagccttcccagaattcaggccttcgtgtccacttccttaccaggataggtacggtgggctgggatatttgcgcaataaaccctccagtctccctctggccaatcccctgacgctaagagatagcatgcaaggagatttaagtcacttccagcaaagctcatggcaacctaaaggggttttccggcaccttgggcaggaggcttctatgatggatgttggcaggactgcggttgtgccacgggcagatggcaggaggggtgtacaaatacattccagaaccaatcttatggatgctgagctattggatgctgactcagatttttaagtctttggagttactggtgctcattctgtgcataaaaacaaaaggtgctggatacttactccaaactatcaactatgagcagaagtgggtttgaactgctgtgccggctctgcttgtgtaaaggataatatttttttgcaaccctcatgatgttatgaattttgaactggatttatgaaacataaaaaataaaa

SEQ ID NO: 46 - protein, 822 aa, Danio rerio
MSSKRPCSIVGSFWMLWIWTATSMVARAVILHPNETIFNDFCKKSTTCEVLKYNTCLGSPLPYTHTSLILAEDSETQEEAFEKLAMWSGLRNAPRCWAVIQPLLCAVYMPKCENGKVELPSQHLCQATRNPCSIVERERGWPNFLKCENKEQFPKGCQNEVQKLKFNTSGQCEAPLVKTDIQASWYKDVEGCGIQCDNPLFTEDEHSDMHSYIAVFGTITLLCTFLHLATFLADWKNSNRYPAVILFYVNACFFIGSIGWLAQFMDGARNEIVCKSDNTMRLGEPSSTETLSCVIIFVIVYYSLMSGVIWFVMLTYAWHTSFKALGTTHQPLSGKTSYFHLVTWSIPFILTVAILANSQVDADSVSGICFVGYRYYEYRAGFVLAPIGFVLVIGGYFLIRGVMTLFSIKSNHPGLLSEKAASKINETMLRLGIFGFLAFGFVLITFGCHFYDFFNQAEWERSFREYVLCEANVTIAHQTNKPIPECAIKNRPSLLVGKINLFSMFGTGIAMSTWVWTKATILIWKRTWFRIIGRSDDEPKRIKKSKMIAKAFSKRKELQKDPEKELSFSMHTVSHEGPVAGINFDLNEPSIEMSSAWAQHVTKMVARRGAILPQDISVTPTGTPIPPPEERNKLWMVEAEISPEMMKRKKKKKKRRKEVRPAGPAADEGNPAYHRREFGPSAVPRLPKLPGHRSLVANLWEQQRQQQEEQDMLPGAFPEFRPSCPLPYQDRYGGLGYLRNKPSSLPLANPLTLRDSMQGDLSHFQQSSWQPKGVFRHLGQEASMMDVGRTAVVPRADGRRGVQIHSRTNLMDAELLDADSDF

SEQ ID NO: 47 - DNA, 2445 bp, Danio rerio
aggagctggttacacacgtgtgtgtagatgtagaacagaaacatgggcaaaagaaggcgatgtaaacaggagattgataatttaactaagaaacagaagaaacatctaaaggaatttggtgaacaacacccttttcacgataaggttgttgaaagaccggagaagactcagattttgcgcttgcctgacagtccacagcgccctgaaccagacagtgaggatgacagtgatgctgagcaaccatctgcatatcagaaactactgtccaccatgattcgaggtgatgaagatgatgttgagagtgaagatgaagaaagcgaggaagaggataacgaggaggaagctgaagttgaaggtgatagtgaagaagaagtggaggacaccgatgtagaggaagggaatgatgctgaggaagttttagaggataaagtggaagaaacatctgacaaattacagaaaaaacaccctgaaaagaccaatggagatgtagaggagaatgaagaggcagagatggagggagagtttacagataaaaagaatgaagctgctttctgcttggagaccaatatgcctgcagaaggagaggaaaacacagcacaacaggaacaggatgaagacatgtttgtgaagcaccaagagattgaactgagcgaagaggaagtgggacgaatctcacaaggatcaaaggtcaagactcaggttaagtgggcaaaactgggtgttctgcagtgcatgagtccattggaacgttttccagccatcggacaggcctcttctgcccctcttccacccattcacaaaaccctagaggcaaattggcatttgttaaacctgccttttggagctgcaacagatgtcacagagctgcaaaaggagatgcttgggctcatgggtacttacagagacctctatttggcaaacctctcccccttgaaggaggctaaggaagtgcgcaacgcatactgtcttcatgccctgaaccatgtattaaaggccaactctcgtgtgcttagaaacaatgccaagttaaaggaatctaaaaatggggatgaggattttcgtgaccaagggcttactagaccaaaggtcttgattttggtgccattcagagatggagcactgagagttgtccagaccttcatgacacttctggagcccaaaggcaagaagacggatgccagtaacaagaaacgcttcaaggaggaatatggagaggagacggatgagaagccacctaatctgcaaaggcctgatgactaccacgctgtcttttctggaaatatagatgatcatttcaggataggtatgtctatcctgagacacagcatgcgcctatacgctcccttctattcctctgacatcatcattgcttctccgctgagtctccgcacagttctgggtgctgaaggggagaagaaaagagaccatgactttctgtcttcaatcgaacttttgattgtggatcaggcagatgttttcctcatgcagaactgggaacatttactccatgtgttggagcatttgaacctgcagccgctggactctcatggtgttgatttctctcgcgttcgcatgtggaacctaaacaactgggcagcttattacagacagacgctggtgttcagtgccatacaagaacctcagatcaccaacatcctcaccaaacactgccacaactacagaggccaggtgtgcagtaaaaccattccaaagataggctcaatctgtcaggtacttgtgcagctgccacatgtttttcagatgtttcactctgacagcttcatggaccaggatgccaggtcccagttctttgtggataagatcctgcctcagtacagagactctgtcatgtccgacactttcatctatgtgcgttcgtactttgatcttgtacgtctgcgaaactacatgaagaaggaagacgtcagttttgttaacattagcgagtactctcagagatcagaggtgtctcgagcgcgacattacttccagaaaggagaaaaacagtttctgctcttctctgagagattccacttttataagagacacactatcaagggtatccacaatctgatcttctacgggttgcccacataccctcatttctacagtgaggtgtgtaatatgcttcaggctggagtcagagagggtggtgcttctgtcagcttcacctgcacggctctgtactctcgatatgatgtgcaccgtctggctgccatcaccggggctgaccgcgccgctcagatgctccagtccaaaaaaaccacacacctcttcatcacgggggaggagaaaaacacatgaagaggtactgcgaaaatctgatgtgacgttttaatctctctgagaaggaagataatctgtgttggtggtagtgggtaaaatgatgtattcccttctgtcatatgactgaatttaatgatgatattccctttcccttttata

SEQ ID NO: 48 - protein, 753 aa, Danio rerio
MGKRRRCKQEIDNLTKKQKKHLKEFGEQHPFHDKVVERPEKTQILRLPDSPQRPEPDSEDDSDAEQPSAYQKLLSTMIRGDEDDVESEDEESEEEDNEEEAEVEGDSEEEVEDTDVEEGNDAEEVLEDKVEETSDKLQKKHPEKTNGDVEENEEAEMEGEFTDKKNEAAFCLETNMPAEGEENTAQQEQDEDMFVKHQEIELSEEEVGRISQGSKVKTQVKWAKLGVLQCMSPLERFPAIGQASSAPLPPIHKTLEANWHLLNLPFGAATDVTELQKEMLGLMGTYRDLYLANLSPLKEAKEVRNAYCLHALNHVLKANSRVLRNNAKLKESKNGDEDFRDQGLTRPKVLILVPFRDGALRVVQTFMTLLEPKGKKTDASNKKRFKEEYGEETDEKPPNLQRPDDYHAVFSGNIDDHFRIGMSILRHSMRLYAPFYSSDIIIASPLSLRTVLGAEGEKKRDHDFLSSIELLIVDQADVFLMQNWEHLLHVLEHLNLQPLDSHGVDFSRVRMWNLNNWAAYYRQTLVFSAIQEPQITNILTKHCHNYRGQVCSKTIPKIGSICQVLVQLPHVFQMFHSDSFMDQDARSQFFVDKILPQYRDSVMSDTFIYVRSYFDLVRLRNYMKKEDVSFVNISEYSQRSEVSRARHYFQKGEKQFLLFSERFHFYKRHTIKGIHNLIFYGLPTYPHFYSEVCNMLQAGVREGGASVSFTCTALYSRYDVHRLAAITGADRAAQMLQSKKTTHLFITGEEKNT

SEQ ID NO: 49 - DNA, 701 bp, Danio rerio
aggcagtggtaacaacgcagagtacgcgggattcatatcagcaatggcgtcccagcagatgagcactaacaatgctcagatgagcgctcagcaagcggcagttttacagcaaacacaagctcaacagctcagtcaacagcaagatttcgaccccgttcacagattcaagatgctaattccaccgctcaaagacagtctacagaatgtcatgaccatcgcgtctctaaattttgcacataatactgctattgacaacggcttaaaaacaaccgagaagggtaatgatgccgcagttcagcggtttgacaaaagtctagaggagttttatgctctctgtgatcagctggagctctgcctgcggctggcccatgagtgcctctcacagagcatcgacagcacgaaacactctccaaatctcgtacccacagccactaaacctgacacggtgcaaacagaatctctgtcttactctcagtatctcagcatgatcaaatcacagatttcatgtgctaaagacatccataacgctctcttagagtgttcaaaaaagatcgctgggaaaggtcagggagcttgtaattagttgctgccaaatctatttcgtttcccctttgtgtttgtgtatgcataactttgatgatgatcaaaaaagtttttttttttttttggggggccccctggggggttaatttaaaaaaaaa

SEQ ID NO: 50 - protein, 179 aa, Danio rerio
MASQQMSTNNAQMSAQQAAVLQQTQAQQLSQQQDFDPVHRFKMLIPPLKDSLQNVMTIASLNFAHNTAIDNGLKTTEKGNDAAVQRFDKSLEEFYALCDQLELCLRLAHECLSQSIDSTKHSPNLVPTATKPDTVQTESLSYSQYLSMIKSQISCAKDIHNALLECSKKIAGKGQGACN

SEQ ID NO: 51 - DNA, 2145 bp, Danio rerio
ggcacgagctgagcttgtaaatcagaaacaaaactgtaagtcggtcggctgcacggttcgattcaagtctctccactgcagtacaaacatgttcacgcaagaggttaggtaaagacagcccgaaacatctcgacatcccaaaacgaacccgacagcgtccacagaagaactttgaggacgcctccgacttcgctttgcattttccagcatgatgtcacaagcagatgcagacatcacgcccttcttcgctgatgataatgaaggggaggggcctgttgaaaatggggtggtgtcacctctgcctgaggacgaggaggaggagtctcccagcggagtcaccgataggagagccataatgactgtgattgtgctctgctacatcaacctgctgaactatatggacagattcactgtggcaggtgtacttccagacattgagcatttcttcggcattggtgatggaacgtcagggctgcttcaaacagtcttcatctgcagctacatgttcttggctcctctgttcggatacctgggtgaccggtataacaggaagctgatcatgtgtgtggggattttcttctggtctgtggtaacacttgccagctcatttatcgggaaagatcacttctgggcactgctgttgacacgagggctagtgggtgttggagaggccagttattccaccattgctcccaccatcatcgctgacctctttgtcaaggaaaagaggactaacatgctgtctatattttattttgccatcccagtgggcagtggtatgggttatatcgtgggctcaaaggtggatactgtggctaaagattggcattgggcacttcgggtgacccctgggctgggactgctggccgtgttcctcttaatgcttgtggttcaggaaccgaagcgtggtgcgattgaagctcaccccgagcacacgctgcacagaaccagctggctggcagacatgaaagcactttgcaggaatccaggttttattctgtccacgttcggcttcacggcagtggcctttgtgacgggatcactggccctgtgggctcctgcctttttatttagagccggcgtcttcaccggagtcaagcagccgtgttttaaagctccatgtgatgactctgacagtctgatttttggcgccatcactgtagtaacgggcatcttgggtgtagcgagcggtgttcaggccagtaaactgcagaggaccagaactcctcgagctgacccgctagtgtgtgcagctggactcctcctcgcggctccctttctctatctctccatcatgtttgcacaggcgagcactgtggccacatatgtctttatcttcctgggagagacattcctttcaatgaattgggccattgttgctgatatcctactgtatgttgtgatcccgacacgtcgctccacagcagaggcctttcagattgtcctttcacacttgctgggtgacgctattagtccctatctcataggagtggtttcagactcaataaaagaatcaaattcgtacatgtgggagtttcgctcacttcagatgtctctgctgctgtgctctttcgtgcccgtcgctggaggcgctttcttcttagccacagccgtctttattgagaaagaccgagatctcgctgagaactatgtgccctctgatgacgcaccaattgttgttccaaggagcggcagatccactaaagtatcagtgtcgagtgttttaatttgagatgacgcaaaagagtggatggctcttgacatgcagggaacagcactctgattggctaaggcagacagaggcagtgtcatcagcagccaatcctggagcagtatcaaacaagtaatgactggactatctcctcaataggaaattttatagtaatttattaagctttttttgttttgtttatatccttaacaccttgatttgggaacatttctaactaacaagaaaatcagaaacctcttacctgaaaaccctagtgtgccgctggaccactttgttgtttcacctggaggagacagaagcacaagggtaaaacaagacaaccggggacatttatttgcctttaattaaaactgtgttttagacttcaagtcgcattttacgtttttttttatcagaagggatgtgaactaaat

SEQ ID NO: 52 - protein, 507 aa, Danio rerio
MMSQADADITPFFADDNEGEGPVENGVVSPLPEDEEEESPSGVTDRRAIMTVIVLCYINLLNYMDRFTVAGVLPDIEHFFGIGDGTSGLLQTVFICSYMFLAPLFGYLGDRYNRKLIMCVGIFFWSVVTLASSFIGKDHFWALLLTRGLVGVGEASYSTIAPTIIADLFVKEKRTNMLSIFYFAIPVGSGMGYIVGSKVDTVAKDWHWALRVTPGLGLLAVFLLMLVVQEPKRGAIEAHPEHTLHRTSWLADMKALCRNPGFILSTFGFTAVAFVTGSLALWAPAFLFRAGVFTGVKQPCFKAPCDDSDSLIFGAITVVTGILGVASGVQASKLQRTRTPRADPLVCAAGLLLAAPFLYLSIMFAQASTVATYVFIFLGETFLSMNWAIVADILLYVVIPTRRSTAEAFQIVLSHLLGDAISPYLIGVVSDSIKESNSYMWEFRSLQMSLLLCSFVPVAGGAFFLATAVFIEKDRDLAENYVPSDDAPIVVPRSGRSTKVSVSSVLI

SEQ ID NO: 53 - DNA, 2213 bp, Danio rerio
tttttttaaaaaagcagaaactaagagaataaagtcgtgacttatttgctgtttgttacttttgctcgctttttattcggtatacactgcgggattgtttcgctcgttgttcttttcggaggcccggttttttattactcaagaaaatccagctcgcgagacccgagcgtctgaaagaagttaaacacagaaagactccgcatccccatgaggcaaccagaagtgccaatcgaagagatttcgtgactgaaccttttgttttgtattcatttttggtgcttcattactttagcagtcagcgatgaagatgatcgttgtgtttacagtatgtatgtctgtggtggttttggcgtcggctcaggctgatcagaagtcaaagaactgcaatgaagtcagaactgcctacagttccaaaggtttcaacgtcaacgatgttcccaacaaaggagtgcagggagcccatctgaaggtgtgtcctcaaggtttctcctgctgtactctggagatggaggagaagttgagtcagctgagccgcgtagacctgaaagttcccgttcaccagctcagctccaacctgcagagtaccttcacccagagacaccgtcactttgaccagttcttcagagagctgttggacaatgcagagaagtctctcaatcacatgtttgtgcggacgtatggcttgatgtacgtcaagaacaaagagctctttgagggtttcttcagtgatctgcgccgctactacagccatggcagcagtgaggtcaatctggacgacatgctggccgagttctggtctgaactgctggagcgcatgtttcgactggtcaacgtccagtacgagttcagcgactcctacatggagtgtgtcagcaggcacacggaccagctcaaacccttcggagacgttccacgcaagctccgtctgcagttgacccgctccttcattgctgttcgcgccttcactcgtggcctcacactcatgcctgatgtggtccgcaaagtctcaacggtgagcgcctcaccgagctgtgtgcgcgcatcgatgaagatgttatactgcccgtactgtagcggtcaggtggcgctgaagccctgcaagaactactgtctgaatgtcatgaggggctgtctggccaatcaggccgatctggacacagagtggaataacttccttgactccatgttaggtctcgctgagaggctggagggaccctttaacttcgagtcggtcgtggaccccatcgatgtgaagatttcagatgctatcatgaacatgcaggagaacagcatgcaggtctcacagaaggtgtttcagggttgtgggcagcccaaactcagcatgggcttccgcagccggaggtctgctaaagactcgggcttccctggccgttttagaccttacagccctgaggccagacccaccactgctgctggcaccaccctggaccgactgctgacggatgtcaagaagaaactgaagcatgccaagaagttctggtccaccctgccggacacagtgtgtgttggagaaaggatcgcccccagtgatgattgctggaatggtacagctaaaagcaggtacgaatctgtggttatgagcagcggtttagccaatcaggtgtctaatccagacgtggaggttgacatcaccaaaccagacatggtgatccggcggcagatcgcagtcctgaaggaaatgaccagctggctgaaagctgcttacaccggcaacgacatctcatttacctccgatgatgccggcagcggagaggaaagcggaagcggctgcgactctccctcttgcgaaggcgacggagatatttacttctccaccccggcacctggtaaacctcgcatcctcaagactccggaggtcgaacaggcctccagcggcacacggttggcaccctgcagcctggcactggccctggcctcccttctgctcaccctgcttactctccaaacaagataaaagcagaatcagcccaagaaagagcaagtgattggacaaatgtttttgaccgcgagagcagaaaactgccacaggacattaaagacactgctttcaggatctccatctgccttccatccggggaagacactcaagttattaatattcacacaacttcagtgtatttttttaatgtttataagagcggactccattttgtgtacagctatacggctccatgcagcattgcgtcgtctgg

SEQ ID NO: 54 - protein, 557 aa, Danio rerio
MKMIVVFTVCMSVVVLASAQADQKSKNCNEVRTAYSSKGFNVNDVPNKGVQGAHLKVCPQGFSCCTLEMEEKLSQLSRVDLKVPVHQLSSNLQSTFTQRHRHFDQFFRELLDNAEKSLNHMFVRTYGLMYVKNKELFEGFFSDLRRYYSHGSSEVNLDDMLAEFWSELLERMFRLVNVQYEFSDSYMECVSRHTDQLKPFGDVPRKLRLQLTRSFIAVRAFTRGLTLMPDVVRKVSTVSASPSCVRASMKMLYCPYCSGQVALKPCKNYCLNVMRGCLANQADLDTEWNNFLDSMLGLAERLEGPFNFESVVDPIDVKISDAIMNMQENSMQVSQKVFQGCGQPKLSMGFRSRRSAKDSGFPGRFRPYSPEARPTTAAGTTLDRLLTDVKKKLKHAKKFWSTLPDTVCVGERIAPSDDCWNGTAKSRYESVVMSSGLANQVSNPDVEVDITKPDMVIRRQIAVLKEMTSWLKAAYTGNDISFTSDDAGSGEESGSGCDSPSCEGDGDIYFSTPAPGKPRILKTPEVEQASSGTRLAPCSLALALASLLLTLLTLQTR

SEQ ID NO: 55 - DNA, 2688 bp, Danio rerio
ctgacggagcagctgagaagccggccagcagtgcagccctttgtccgctaaataaacctgatcatttttattaaaaagtgtacagtttttcttctcgctggcctttctaactttaccagatacctcaatcatgcacgtgatcaagagagatgggcgccaggagcgtgtaatgtttgacaaaatcacatcacgcatccagaaactttgctatggactcaactctgactttgttgatccaactcagatcaccatgaaggtgattcagggcctttacagcggcgttactacagtagagctggacacactggctgctgagatcgctgccaccctcaccaccaaacaccctgactatgccatcctagcagcccgcatcgccgtgtctaacctgcataaagagactaaaaaggttttcagtgaggttatggaggatctctacaactacgtcaatcccctgaacagccgccactcgcctatgatctccaaggagacgcttgatattgttttggccaacaaagatcgcctgaactcagccatcatttatgacagggatttctcttacaacttctttggcttcaaaacattggagcgctcatatctgctgaagatcaatggaaaggttgccgagcggccgcagcacatgctgatgagggtgtccgtaggcattcataaggaagacattgcggctgctatcgaaacctacaacctgctgtctgaaaagtggttcactcatgcttctcctactctgtttaatgcgggcaccaaccggcctcagctctccagttgcttcctgcttgctatgaaagatgacagcattgagggcatctatgacaccctgaagcagtgtgcgctcatctccaagtctgccggtggcatcggggtggcagtgagctgcattagagccactggcagctacattgcaggaacaaatggtaactcaaatggcctggttcctatgctccgtgtctacaacaacaccgcacgctatgtggaccaagggggaaataagagaccaggagcttttgccatgtacctggagccttggcattttgacattttcgacttcctagagcttaaaaaaaacactggtaaagaggagcagagagccagagatctgttttacgccctctggattcctgatctcttcatgaagcgagtggagaccaatggggattggtctctgatgtgcccaaatgactgtcccggcctggatgagtgctggggagaggagtttgagaagctctatgcaaaatatgagcaggagggcagagcgaagcgtgtggttaaggctcagcagctttggtatgccatcatcgagtctcagactgagaccggcacaccatacatgctttacaaagacgcctgcaaccgcaagagcaaccagcagaacctgggcaccatcaaatgcagcaatttgtgcacagagattgttgagtacaccagtgccgatgaggtggcagtttgcaacctggcctccattgcacttaacatgtacgtcacctcagagcgaacatttgacttccagaagttggcctctgttacgaaggtcattgtgaagaacctgaacaagatcattgatatcaactactacccagttaaagaggctgagaactccaacaagcgccaccggcccatcgggatcggcgttcagggtctggccgatgccttcatcctcatgcgtttcccatttgagagtgcggaggctcaactcctcaacacgcagatctttgagaccatctattacgctgctctggagtcaagctgtgagctcgctgctgaatacggcccgtatcaaacctacgccggctgtcctgtcagcaagggtatcttgcagtatgatatgtgggaaaagacacctacagacttgtgggactgggcagcgcttaaggagaagattgcaaagcatggggtgcgtaacagtctgctgctggccccaatgcccactgcctccacagctcagattctgggcaacaacgaatccattgagccgtacaccagcaacatctacacccgcagggtcctgtctggagagtttcagatcgtgaacccgcatcttctcaaagacctcacagaacgaggactctggaacgaggaaatgaaaaaccagatcatcgctcagaacggatccatccagaccattcccgcaatccctgatgacctgaaggagctgtataagacagtgtgggagatttctcagaagaccattttaaagatggctgcagatcgaggagcctacattgaccagagccagtcgctcaacatccacatcgctgagccaaactacggcaaactcaccagcatgcacttctacggctggaagcagggtcttaaaacaggcatgtactacctaagaactaaaccggctgcaaaccccatccagttcaccctcaacaaagagaagctgaaggagacacagaaaacgaccagcagtgaagacgaggaaaccaaagagcgtaataaagctgcgatggtgtgttcactggagaaccgtgacgagtgtctgatgtgcggatcataaactcaactcctgcactcggacacaatcaatagcatcatttattcatttgcaacttgtacagtattaaaccttttttaaatggccatggtgaagtacagaattattcatattaattaactttcccttttgtatgcgaataatatagtgtacatttgggaattatatttttggat

SEQ ID NO: 56 - protein, 794 aa, Danio rerio
MHVIKRDGRQERVMFDKITSRIQKLCYGLNSDFVDPTQITMKVIQGLYSGVTTVELDTLAAEIAATLTTKHPDYAILAARIAVSNLHKETKKVFSEVMEDLYNYVNPLNSRHSPMISKETLDIVLANKDRLNSAIIYDRDFSYNFFGFKTLERSYLLKINGKVAERPQHMLMRVSVGIHKEDIAAAIETYNLLSEKWFTHASPTLFNAGTNRPQLSSCFLLAMKDDSIEGIYDTLKQCALISKSAGGIGVAVSCIRATGSYIAGTNGNSNGLVPMLRVYNNTARYVDQGGNKRPGAFAMYLEPWHFDIFDFLELKKNTGKEEQRARDLFYALWIPDLFMKRVETNGDWSLMCPNDCPGLDECWGEEFEKLYAKYEQEGRAKRVVKAQQLWYAIIESQTETGTPYMLYKDACNRKSNQQNLGTIKCSNLCTEIVEYTSADEVAVCNLASIALNMYVTSERTFDFQKLASVTKVIVKNLNKIIDINYYPVKEAENSNKRHRPIGIGVQGLADAFILMRFPFESAEAQLLNTQIFETIYYAALESSCELAAEYGPYQTYAGCPVSKGILQYDMWEKTPTDLWDWAALKEKIAKHGVRNSLLLAPMPTASTAQILGNNESIEPYTSNIYTRRVLSGEFQIVNPHLLKDLTERGLWNEEMKNQIIAQNGSIQTIPAIPDDLKELYKTVWEISQKTILKMAADRGAYIDQSQSLNIHIAEPNYGKLTSMHFYGWKQGLKTGMYYLRTKPAANPIQFTLNKEKLKETQKTTSSEDEETKERNKAAMVCSLENRDECLMCGS

SEQ ID NO: 57 - DNA, 3662 bp, Danio rerio
aactattttgccatttcttgtggtctactcttaagcaagatcggcttaacggttacatttaaagggcatttgagatctggagcctagaagacgctccttcaacatggcatcatcacaagtaccagcagccaaaaaagatgagaagggcagaaacatacaagtggttgtacgatgcagaccctttaacacagtggagcgtaaatctggctctcacactgttgttgaatgtgaccagaaccggaaagaggtgattatgcgtactggaggtgccacagacaaagcagcaagaaaaacatacacttttgatatggtttttggcccttctgccaaacaaattgaagtttataggagtgtggtttgccccatattagatgaagttatcatgggctataactgcacaatctttgcatatgggcaaaccggaacaggcaaaaccttcacaatggaaggtgaccgatcacccaatgaggagtttacttgtgaagaggaccctctggctggaataattgccaggactcttcatcagatctttgaaaaactgtctaataatggcacagagttctcagtgaaggtgtctctgctggaaatttataacgaggaactgtttgacctgctcagccctgctgctgatgtcacagagagattacagctcgtgtgtgatcctagaaataaaaagggtgtgaccattaaaggtctggaggaaatcaccgcacacaataagaatgaggtgtatcagatcctggagagaggagcagccaagaggaagactgcctccacactcatgaatgcctattccagtcgatcccactctgtgttctctgtcactattcacatgaaggaaatcacactggatggggaagagctagttaagattggtaaactcaacttggtggatcttgcaggtagtgagaacatcggacgatctggtgctgtggataagcgtgcacgtgaagctggcaacatcaaccagtctctgctaactctgggtcgtgtaatcaaggctttggtggagagagggcctcacgtgccctacagagagtctaaactcacccgcatactgcaggactcgctaggaggacgcaccaaaacctctatcatcgccactgtgtctcctgcctccatcaatctggaggaaactctgagcactttagactatgctaacagagctaagagcatcatgaataagcctgaggtcaatcaaaggcttaccaagagaaccctcatcaaggaatacacagaggaaattgagagactgaagagagatttggctgccacccgtgacaaacatggagtgtacctttccgttgataactatgagaccttgaatggtaaaattgtgtctcaggaagagcagattacagagtacactgagcggattgctgctacggaggaggagctcaaaaagattatagacctgttcacagatagtaagcagaaattggaacagtgcactgaggacctgcaagacaaaaaccagagactggaggaggctcacaaagacctatcggagaccaggcgccgcctcaatcaggaggaattcatcagtacacagcttcaaaccaatgagagtcacctctataacactgctgaccagttgttgagcactgctgaggccagcacacaggatgttggtggtctccatgctaagctacaaaggaagaaggatgtggagcttcataacagtaagggtcacgagagcttctcccagtgcatggagaactgctacaacagcatgcagacctcacttgaaggagcagaacccaaaacatgctgctatgattgattattaccgctcctctgtgggtgagctgcttgaacccaatggcaaggaaggagcagagcccaaaaccatgctgttatgattgattattcccgctcctctgtgggtgagctgttgaacaccaatggcaaggtgtttaagaagactttgggtgctgtgtgcgagtcttacagcagtattaaaggagctgtggaagaaggagtagagcggtgtaaggagcaagtgttaaatcaggagaaactctctcaggatgctcaaaacagcatcctggaaattctggatgaacacaaacagcatcttgaggaggttctggttgcccaggcggtgccaggtatcaggtctgtcatgtccatgaatgacaatttgaagcaaacccttcacaaataccacaatctggctgagcagatgcagggtgtgaaagcagatatgatgacgttttttgatgcgtacactgaatcactagccagtatgcgagagtgcgctttgcagggttttgacacactgcgtgccgaacatgacaaactcaaacaacaaatcagccaagctggaaacagccatcaagtgcgtgtggccgagctggttcagtgtttgcagaaccaaatgaatctgctggcggtggacactcagaatgactttgagggtctttcacaagcagcatctgcccagattcctccacttggaaacattacagagctccatagagagtaaatgcacagtggctgaggagcaggctgtgtctgttcgtgctcgactgggttcctctgttcatggagtgatttcagagatgaatagtgtgactaaggagggagagagggcgctggaggagtgtgctggttattgtggacacctgcagacatcgctggactcgctggctgagtcaggactcaagtggtgcgatgaagctaaaggcttgactgagagtaaagcccaggagcaactcaaactcatcagacagacagacacggctgtgcaagacctgctgaagtctgtggaggaaaagggtgagaaagctgttcaggactgtgaagccagattgggtcaaatgcagcaggaaatggaagcaaccctgggtcgtgtggaaatgcagaccagcaaagatgaagcgacattgcaggagcacagagagaccctgtcctccatcaacactcaggcactggacaccgtacacaacttcatcagctcagagctacggcaggatttaccaacaggaacgacccctcagcgcaaagagtacatgtacccacgtgtgctgagcaggccgaggagtagagagtagctggaagaggagttcagagctcagcaagagcagcttcagtctgaactgaagccgtgtgagatcgtaatggaggtggaggaggagaaacctgtcgaccaggactctctggaggatgacgtcagtgtttctagtgatggaaacaacactgaacagtcgtgctctgatgagaacctgatatgttatgagaatggaaggattccttttttcaagaagaaaagcaagaaggaaaatggcagtaaatcactgaaccgttcaaaggtggagaatgacagcatgtcaacaccaccccgctctaaacttccactcagatgtcagaattaacctaaattatgttgccaatattactcccttcgtgtattaatcttccaagtccttttgtgtcttaaacgttgcattttttaactacgcttgtcttttttaaacatcgattatcaatgtatttgtcttatgtctgtccacacaagtcagttataacttcagggtgatatcgtactgttacttttcctgtttcactagcgtttaggcttgtttgtatttgttcaaggttttgtatgtttaattaaagactaaaagaaaatggtccactccatctctcaaataatataa

SEQ ID NO: 58 - protein, 983 aa, Danio rerio
MASSQVPAAKKDEKGRNIQVVVRCRPFNTVERKSGSHTVVECDQNRKEVIMRTGGATDKAARKTYTFDMVFGPSAKQIEVYRSVVCPILDEVIMGYNCTIFAYGQTGTGKTFTMEGDRSPNEEFTCEEDPLAGIIARTLHQIFEKLSNNGTEFSVKVSLLEIYNEELFDLLSPAADVTERLQLVCDPRNKKGVTIKGLEEITAHNKNEVYQILERGAAKRKTASTLMNAYSSRSHSVFSVTIHMKEITLDGEELVKIGKLNLVDLAGSENIGRSGAVDKRAREAGNINQSLLTLGRVIKALVERGPHVPYRESKLTRILQDSLGGRTKTSIIATVSPASINLEETLSTLDYANRAKSIMNKPEVNQRLTKRTLIKEYTEEIERLKRDLAATRDKHGVYLSVDNYETLNGKIVSQEEQITEYTERIAATEEELKKIIDLFTDSKQKLEQCTEDLQDKNQRLEEAHKDLSETRRRLNQEEFISTQLQTNESHLYNTADQLLSTAEASTQDVGGLHAKLQRKKDVELHNSKGHESFSQCMENCYNSMQTSLEGAEPKTCCYDMIDYSRSSVGELLNTNGKVFKKTLGAVCESYSSIKGAVEEGVERCKEQVLNQEKLSQDAQNSILEILDEHKQHLEEVLVAQAVPGIRSVMSMNDNLKQTLHKYHNLAEQMQGVKADMMTFFDAYTESLASMRECALQGFDTLRAEHDKLKQQISQAGNSHQVRVAELVQCLQNQMNLLAVDTQNDFEGLSQAASAQIPPLGNITELHREMTLRVFHKQHLPRFLHLETLQSSIESKCTVAEEQAVSVRARLGSSVHGVISEMNSVTKEGERALEECAGYCGHLQTSLDSLAESGLKWCDEAKGLTESKAQEQLKLIRQTDTAVQDLLKSVEEKGEKAVQDCEARLGQMQQEMEATLGRVEMQTSKDEATLQEHRETLSSINTQALDTVHNFISSELRQDLPTGTTPQRKEYMYPRVLSRPRSRE

SEQ ID NO: 59 - DNA, 2808 bp, Danio rerio
ccacgcgtccgaaacaaagcggattgctagcgagctaacagttaacgttaccccagtagtatctgtgcacacaggggcataaaattaagagtgcgattaggggatgttttaaaaaccgtgcgcacagtcagggtttaattagatgtttctttctgcattatgtgcaattttatagttttaatgcatcgtatgtaatagctaactggttagccctcagtgagacagctttgattcgagggagcaaaatgaaactaaactataatggtccgcgttgtaagtgatagttcaataattaagcgatgtgacgttatcttagctgaccagctttgttttattttgacgttattatcatgtaataaccaaacaaaatgaatgtacacatctttacgagtcactcagatagtcaaataatagcgtgttagtagataacgggttgattaaacatacaaagaggaggcgttaagtgctaacgttagtaatcagctaacttaaaacgactgtcatgaaaaaataaaccccagtgaaagcatttaggggggaaatgttcaatctgatggcgaactgttgcaactggttgaaacgatggagagaacctgcaaggaaggtgacattagtaatggtgggattggacaatgctggaaaaacggcgacagtcagaggaatccagggagaaagccctttagatgtagcacctacagtgggcttttcaaaggtggacctgaagcagggaaagtttgaggtcacaatctttgacctgggtggcgggaagcgtattcgtggcatttggaaaaactactactctgaatcttatggagtggtgtttgttgtggactccagtgatgtccagagaatccaggagaccagagacaccatggctgaggttctccggcatcctcgtattgcaggcaaacctgtcttagtactggctaataaacaggaccaagatggggcaatggctgaagcagatatcattgagaccctgtcattggagaagctagtcaacgagaataaatgcctttgccagattgagccctgttcagctgtcttgggttatggtaaaaaggttgacaaatccatcaaaaatggccttaactggctcctaaataacattgccaaggactatgaggcaatttcagaacgtgtgcaaaaagatacagccgaacagaaggctcaggaagaacaagacaagaaagaaagagcagagagagttcggcggatcagagaagagagagaccgacaggaacgggaggaagcagagcgagaaggaaggacattaaaagaggaagagctagatgatgtcaacatgttcaacccctttcaaccaataaacaatgttttgactgagaaccaagacagactaaacagagaaaaagagatgcaaagacagagggaaaatggccagcagggcagtgtgcaagaacagatagcacttcaagatgaggaggaagaagaggaagatgaagagagtgagagacagactccagaaagcacagaatcaggagcagtggaccagaccaaaaagaaaactaggaaactacggttgaaacgcaaacacagagtcgaccctcttaggatggaagaagcagcacccaaaagccccacgcctcctcctctgccagttggatgggcaactcctaaagtttctaggctccccaaacttgagcctcttggagatacaaggcattctgatttctatggcaaacctctcccacctgtagcaattcggcagaggccgaacagcgatactcatgatgtcatttcctaacactacctctacttccccaacccccccttcactggtttacagttcttaaggttccctgccaatcaaagtattgttttgttcttatcttcaagcacgggcagtgtagcaagctacgcagatgtaattgcccataagagaggaacttgatacccaatctgcattcccagatggatcacaagaactccagcaaccagtgactgatagtcagcagatggacattcacttcatgacatgttcagaaaacgtagaatggttcaatatttaggattttgttgagctgtttgttttatccaaatactttaaatcagtattaacagacttttgaaattaagtatgtttttaatgcgttgcaaaaatgcattacagacctattttttaagataactgaaaaatatgtacatacaaaaagtttaaatttgtgtacttgaaattgttcttctggtccacatgcataccttattattctgcgcatgtaatgtgcaatcgattgtgacacagtttgcctttaaatgatcagaaatgtttcttttttctctctcagcatatagtttcatacactgtgctgtcgtaattttgagaatgacattcatactatctagtagcaccagcaacagataccgagatcttgataaagtgaactttttgcatgagatgtgaaacatttttctacaagctcgttattgagttttattgtcacatttggtacatcatgctgtatcagggaggaaatggtcattgaacgataatttcatcaattactgatttaaatatattccactgtagttgtaacacggctcaacctgtattacgtatactgttggacgctgctttcagatttattttatgctgttacagtttttttttattgtttcagtttgttggatgttttgtattttacaccacacactaatgtttagttgatgaaaattctgatgtcatccattttgctctgctttaagtacttgcgccttttgtactcggtttacatttaatctaactgtcaaataaataaaagatatttttactctgcaaaaaaaaaaaaa

SEQ ID NO: 60 - protein, 407 aa, Danio rerio
MFNLMANCCNWLKRWREPARKVTLVMVGLDNAGKTATVRGIQGESPLDVAPTVGFSKVDLKQGKFEVTIFDLGGGKRIRGIWKNYYSESYGVVFVVDSSDVQRIQETRDTMAEVLRHPRIAGKPVLVLANKQDQDGAMAEADIIETLSLEKLVNENKCLCQIEPCSAVLGYGKKVDKSIKNGLNWLLNNIAKDYEAISERVQKDTAEQKAQEEQDKKERAERVRRIREERDRQEREEAEREGRTLKEEELDDVNMFNPFQPINNVLTENQDRLNREKEMQRQRENGQQGSVQEQIALQDEEEEEEDEESERQTPESTESGAVDQTKKKTRKLRLKRKHRVDPLRMEEAAPKSPTPPPLPVGWATPKVSRLPKLEPLGDTRHSDFYGKPLPPVAIRQRPNSDTHDVIS

SEQ ID NO: 61 - DNA, 1730 bp, Danio rerio
cctaacaagtgtgcaaagccatctttccctgaagtctttcctattgacggacaagctgttcaaccaataaagcacaggtttttgctcgggatacatctttcttccatttcgacaatcaacttttatccaacttccaagaacttccatctgaggaggcgctttgcatttcaatttcttggaggaggaaggatggatgtgagaatgaaccaaggacacctacttctggcagtgaccctcatcgtctgcaactcacagctgctggtggtcgccaactcgtggtggtcattagccatgaaccccatccagagaccggagatgtacatcattggagcacagcctctgtgcagccagctgacgggcctatctcagggtcagaggaagctctgccagctctatcaggaccacatggtttatattggagagggggcgaagacgggcatcaaagagtgccagtatcagttcagacagaggcgatggaactgcagtacagtggacaacacgtcagtgttcggccgcgtcatgcatataggcagccgagaaacagcttttacgtacgccgtcagcgcagcgggtgttgtgaatgctgtgagtcgagcgtgccgtgagggtgagctttccacctgcggctgcagtcgagcggctcgtcccagagacctgcccagagactggctgtggggcggctgcggggacaacgtcaactatggctaccgcttcgcccgggagtttgtggacgctcgtgaacgtgagaagaactacccacgcggatcggtggaacacgcacgcacgcttatgaatctgcagaacaatgaagccggaagaatggcggtgtataatctagcgaatgtggcctgcaagtgtcatggcgtctcaggctcgtgcagcttgaaaacctgctggctccagctggccgacttccggcgtgttggagaattcctgaaggagaaatacgacagcgccgccgccatgcgcattaaccgacgtggaaaactagagctggtcaataatcgattcaacccaccgacaggtgaagatctggtctacatcgaccccagcccggattactgcctgcgcaatgaaaccactgggtctctgggcacccaaggccgcctatgcaacaagacctcggagggtatggacggctgcgagctcatgtgctgcggccgcgggtacgaccagttcaagacctacaaacatgagcgctgccactgcaagttccactggtgctgctatgtcaagtgcaaacgctgcacgtcactcgtagaccagtttgtgtgcaagtagcagacgtgagaactgggggacagacgcactgagcaattaagcaggagaagaaacgggacccttacggacccagaggggcaacttagagataattaaatgtaaaaatgatatattaaatagcaacaaattaaaagtatataaataagtgtacgtgtgccgttgatagtaatttaatgacctgaggatccacgttggacgtcattgatgaaaaaggaggcaccggtgtgatgcattccggctcaagctgtgtttctcttatatagagaggaaggaactcttgacttgtgctacaagaaactctttagagactcgaggaaaaggcgcagaaataggatggggaacatcaagggcgcatcacccacccattgccttccaattcttaaacacacacacacacacacacactccaccttgctgatgtcagagctcttaagaactttcggaatgcg

SEQ ID NO: 62 - protein, 363 aa, Danio rerio
MDVRMNQGHLLLAVTLIVCNSQLLVVANSWWSLAMNPIQRPEMYIIGAQPLCSQLTGLSQGQRKLCQLYQDHMVYIGEGAKTGIKECQYQFRQRRWNCSTVDNTSVFGRVMHIGSRETAFTYAVSAAGVVNAVSRACREGELSTCGCSRAARPRDLPRDWLWGGCGDNVNYGYRFAREFVDAREREKNYPRGSVEHARTLMNLQNNEAGRMAVYNLANVACKCHGVSGSCSLKTCWLQLADFRRVGEFLKEKYDSAAAMRINRRGKLELVNNRFNPPTGEDLVYIDPSPDYCLRNETTGSLGTQGRLCNKTSEGMDGCELMCCGRGYDQFKTYKHERCHCKFHWCCYVKCKRCTSLVDQFVCK

SEQ ID NO: 63 - DNA, 1624 bp, Danio rerio
ggcacgagcttgcgtttgtcagaccgcggcgaatctgctccacgcatttacagaaaccgagcaaaatcgcatccaagcatcgctctatcgccgtcacatcttcttcacatgcggggctgaactgagtttgcgcgtttgagacaccccggagaggattgcgtgcggcgttatattcgtattgtgcgtcatttctgcatttctgcgtttccacccaggcaatatggcaacaccagccgctgtaaacccatcggaaatgggcacggatttgccggggccggtgtctatgccaggtgctgttgttggtgctggtcaggtcaggatgacgggtgcaatgccaggacgaggaggcaagcggagatcagcggggatggattttgacgacgaggacggcgagggacccagcaaattttcaaggtatgatgatgatcagattcctggtgataaggagcgttacgccagagagaatcacagcgagatcgagcggcgcagacgaaataaaatgacgcagtacatcaccgagctgtcggacatggtacccacctgcagcgccctggccagaaaaccagacaaactcaccatcctgcgcatggccgtctctcacatgaagtccatgaggggaaccggaaacacctcaaccgacggcgcctacaaaccctccttcctcactgaacaggagctgaagcacttgattctggaggcggccgatggcttcttgttcgtggttgcggctgagacgggacgggtgatctacgtgtctgattcggtgactcctgtcctcaaccatccgcagtcagagtggttcggtagcacactgttcgagcaggtccatccagacgacgttgacaaactgagggagcagctgagcacgtctgagaattcaatgacaggtcgtattctggacctgaagacggggacagtgaagaaggaaggtcaacagtcctctatgaggatgtgtatgggctccaggcgctccttcatctgcagaatgaggtgtggcagtgcacctttggatcacatctcattgaatcgattgtccagcatgagaaagagatacaggaatggtctcggtccgtctaaggagggtgaagcgcagtactctgttgttcattgtaccggctacatcaaggcctggccgccagcgggcatgactattccagatgaagacacagaagcaggtcaaactagcaaatactgcttggttgctattggaagactgcaggtgaccagctctcctgtttcaatggatatgaacggactttcagttccgactgaattcctttcacgccacaattcggatggcatcatcacattcgtggaccctcgatgcatcaacgtgattggctaccaacctcaggttcttcttggcaaagacattctggagttctgccacccggaggaccagagccaccttcgggagagtttccagcaggtacgccaaaaccagattaaacccgtcagccaacaccaatcacatatttcccacatgatgtttttttaacagatcaaggaatatttcacagtgtttcctataatatatattttttcttctggagagggtcttatttgttttatttcggccagaataaaagcagtttttaataaaaaaaaaaaaaaaaaaaaaaaa

SEQ ID NO: 64 - protein, 425 aa, Danio rerio
MATPAAVNPSEMGTDLPGPVSMPGAVVGAGQVRMTGAMPGRGGKRRSAGMDFDDEDGEGPSKFSRYDDDQIPGDKERYARENHSEIERRRRNKMTQYITELSDMVPTCSALARKPDKLTILRMAVSHMKSMRGTGNTSTDGAYKPSFLTEQELKHLILEAADGFLFVVAAETGRVIYVSDSVTPVLNHPQSEWFGSTLFEQVHPDDVDKLREQLSTSENSMTGRILDLKTGTVKKEGQQSSMRMCMGSRRSFICRMRCGSAPLDHISLNRLSSMRKRYRNGLGPSKEGEAQYSVVHCTGYIKAWPPAGMTIPDEDTEAGQTSKYCLVAIGRLQVTSSPVSMDMNGLSVPTEFLSRHNSDGIITFVDPRCINVIGYQPQVLLGKDILEFCHPEDQSHLRESFQQVRQNQIKPVSQHQSHISHMMFF

SEQ ID NO: 65 - DNA, 1135 bp, Danio rerio
ctaaagggcaagcagtggtaacaacgcagagtacgcggggggggctcgatgaccgcggctgcagcagcggacgtgtttgaaaacatcaggaggaattgaaggatgatttgggcgtcagcgctgatcttcatcaccgcgctcagctccggtgtgtgtgatgacggacacgagatggaggagttcctcaagagggagtacacactgtccaaaccgtaccaggatgtgggcgtgtcgggctcgtctcattgggagctgatgggagacgctctggtctcctctgattatgttcgtctgactccagatcagcagagcaaacagggagccatatggagccgcatgccttgtcacctgagcgactgggagctgcaggtccactttagggttcacggtcagggaaagaagaacctgaacggtgatggtctggccgtctggtacactaaagagcgcatgcagagagggccggtgtttggaaacagggacttcttcacaggccttggtgtgtttgtggacacgtatcccaacgaggagaagcttctggaggctcataagaagcggtatactccacgcacacagcgtatattcccgtatgtgttggctatggttggcaacggcagcatcagctatgatcacgatcgggatgggcgacccacagagctgggcggctgcaacgccatggtgcgaaaccagaaacacgaaaccttcctcttcatcagatacgtccggcgccgactcacggtcatgatggatatcgacgggcagcatgagtggagagactgtctggatgttcctggtgttcgtctgccgcagggcttttacttcggggcttcagctgtcacaggagacctttcagataatcatgacctgatctccatgaagctctaccagctgacgatcctgcgcagcaagcaggaggacgaggagcaggaggaggttctggttcccagcgtggacaacatcgacctgctgaggcctcccgtagatgaggagagtgtgagcagcgtgggcatcttcttcagcgtgctcttcactctgctgggggtgtttctgctggtggtggtgggactggtgctgtatgaacactggagcgagagccgccgcaagcgcttctactgacacacacacacacacacacaca

SEQ ID NO: 66 - protein, 336 aa, Danio rerio
MIWASALIFITALSSGVCDDGHEMEEFLKREYTLSKPYQDVGVSGSSHWELMGDALVSSDYVRLTPDQQSKQGAIWSRMPCHLSDWELQVHFRVHGQGKKNLNGDGLAVWYTKERMQRGPVFGNRDFFTGLGVFVDTYPNEEKLLEAHKKRYTPRTQRIFPYVLAMVGNGSISYDHDRDGRPTELGGCNAMVRNQKHETFLFIRYVRRRLTVMMDIDGQHEWRDCLDVPGVRLPQGFYFGASAVTGDLSDNHDLISMKLYQLTILRSKQEDEEQEEVLVPSVDNIDLLRPPVDEESVSSVGIFFSVLFTLLGVFLLVVVGLVLYEHWSESRRKRFY

SEQ ID NO: 67 - DNA, 2074 bp, Danio rerio
tgttctgtgagcgcgtgctggagctgcggcagacatgaagagaccgaagttgaagaaggcgagcaagcgcttatcatgcgctaaacgctacaaaatacaaaagaaggttcgggagcacaatcgtaaattaaagaaagccgcaaaaaaacaaggcataagtcgaaaggccaaaaaggatattggagtcccaaacagtgcgccttttaaagaggaagtgcttcgcgaagctgagcagagaaaacaggagcttgaaactctgaaagagcaaaacaagatcgtgaagcagcaagaaaaggctgccaagcggaaaaaggagaaagatgcagccagttcagtaaaagagcctgcagccaagaaagctaaaaaggcagccaaaatcaaagaagcaagagcagccatagttaaagtgaagagcgctaaaacattcaaatgccaagaactcaacaaggtgattgaggcgtctgatgtgatcgtggaggttttggatgccagagatcctctgggctgccgctgtcctcaactggaggaaatggtcctgaaacacgagggaaagaagaaactcttgttcatattgaacaaaatagatcttgttcctaaagataacctagagaagtggctccactttcttgaagccgagtgtccaacttttctgttcaaatcatcgatgcaactaaaggacagaacagtgcaacagaagaggcagcagcgaggaactaacgcagttctggatcacagccgagcagcttcctgttttggaaaagactttctcctacagacacttaatgacttggcgaacaagaaagagggcgaaaccatgctaaaagttggcgtcgtcggttttcctaacgttggaaagagcagcatcatcaacagtatgaaagaaatgcgagcgtgcaatgctggcgtacagagaggattgactaggtgtatgcaggaagtgcatattaccaagaaagtgaagatgattgacagtcccgggattttggcggccctcagtaaccctggaagtgcgatggccctcaggagcctgcaggtggaggagaaagaggagagtccgcaggaagccgtcaggaatctactcaaacagtgcaatcagcagcatataatgctacagtataatgtcccagactacagaagctccttagagtttttgaccacctttgccatgaagcacgggctcctgcagaaaggtggcgttgcagacactgaactggcagccacaacattcctcaacgactggacaggggctaagctgagttactacagcagagttccagagcgacagggtcttccctcttacctgtccgacgccatcgtgattgaactgcagtcagatgtggatatggatgctgtgaagaaggggaacgagaatgtaaagagaagtgttcgctttccaaacctcgcgagctgcataagctttgactccagtggtcccactgctggagtgctggatgtcagtgagctgcctaaagagatcttaacaaaggcagcaacaacaacagatgcagaagaagagaagatggatacgacaaccaacacagatgagcctgaggccgaaagccatatttcatcaaccgtggaaccgattcaggaaccaacagaaaagagaaaagataaaccagcaaaagaagtgaagtttgtaccagtcaacactgacctgacgtcaatgcagaacaagaacaatgaggatgcatacgatttcaacacagactttgtgtaaaaattcactataattatttacatggactgagaggaattaaatagtaaaatgcactcagttgcagtgcatctcatgtttgtatgtaataatacataaaatgtttgtttcactaaatacaaggtaaaactcttacattgcttacaaaacttatactgagctttattcaatacttggttagaaccattttcccctttatgtacaaattagcacactttccccttcagatttgaagctgaaatattcatgtaacagaaacgtggtgtcaaaaatagacatttgaactatcacaggtttgtcttaaagtcctgaacaagtcgactacactacctggcaaaaaaaaaaaaaaaaaaaaaa

SEQ ID NO: 68 - protein, 561 aa, Danio rerio
MKRPKLKKASKRLSCAKRYKIQKKVREHNRKLKKAAKKQGISRKAKKDIGVPNSAPFKEEVLREAEQRKQELETLKEQNKIVKQQEKAAKRKKEKDAASSVKEPAAKKAKKAAKIKEARAAIVKVKSAKTFKCQELNKVIEASDVIVEVLDARDPLGCRCPQLEEMVLKHEGKKKLLFILNKIDLVPKDNLEKWLHFLEAECPTFLFKSSMQLKDRTVQQKRQQRGTNAVLDHSRAASCFGKDFLLQTLNDLANKKEGETMLKVGVVGFPNVGKSSIINSMKEMRACNAGVQRGLTRCMQEVHITKKVKMIDSPGILAALSNPGSAMALRSLQVEEKEESPQEAVRNLLKQCNQQHIMLQYNVPDYRSSLEFLTTFAMKHGLLQKGGVADTELAATTFLNDWTGAKLSYYSRVPERQGLPSYLSDAIVIELQSDVDMDAVKKGNENVKRSVRFPNLASCISFDSSGPTAGVLDVSELPKEILTKAATTTDAEEEKMDTTTNTDEPEAESHISSTVEPIQEPTEKRKDKPAKEVKFVPVNTDLTSMQNKNNEDAYDFNTDFV

SEQ ID NO: 69 - DNA, 1584 bp, Danio rerio
ttttatgttcctgtggagtccggaagccagtagtgtcccgtgttgcaaatcggaatttataaacagggctctccttgagatgttctcagggtttcaggaacggtacgaccatgaggacgagctgtacagggatgaagaagactcatcaggtttgtctgattcggacagcgagttggaattccggcttcattcccagctgcactacaacgcagagttccaggaaaaccatgaggaatcaagcaaagtccaaccattggtcccacaaactcaacttcagccggaaaccatgcctcctgctccacctgtagatgttatcgtgattgacttcagggcagattcttttacagtgtcagacagcacagaggatgatgagagtgtgtgtgctaataaagttcagtcattaaaacattgtaaaaggaagtcacagcccagtcgcttccgttcaccttctccgcctcaggttcaagcaggaaggagctctccggatgatgttgtggtgctggattctgatttagaacaatcctcttctgaatctgaacctccttttgtggaggatttggactcggatacagactctgattcagactctgatggactggagaactggatgattctgggcaaggggagacaagatgaagatcagagcatccagctgaacctcgcgcagagtagttttgtgaatacagaatatgtaaattacagagggcaagagcaatgtggactgtatcagaaaaggataaacaggctcagatctataataaagggcacggcccgaggcatgtcccaaaccgctccacatacagatactacactgaaaaatcatcacgtgtcgcaactgcaataaactggtcatctctccaaaaactgccccacattaaagaaggtgccgtgctgctcattatgtgggcttcggggacacttactgaggacctgtccaaacagtcactgctcgaactgttcgctgcccggcccacctctgatgactgtctggagagagccttttggtacaaaagatgccaccgctgcggcatgaccggacacttcatcgacgcctgtccacaaatctggagacagtaccaccttacaaccactgcaggacccatccgcaagtctgctgatccaaaggcctgccagaaacgtgcctattgttataactgctccaggaaaggacactttggccatcaatgctctcagagaaggatgtacaattggtcctatccatccttgccagttatcacctattatgacacagtgaatgacatcaaatgccgggatttccgtctgaaaaagaaggccagagaaatgcaggacgccggactgatttctcctgatggaggcgttgtaacatttacccctcaaccacccagaaagaaacaaaaagtcagccacagtccacatccgtactcaaacaacacaaacaaccaccacacaccgaagaaacgcatcgcacacacacccaaacacaatccacaaacaaaccaacaaaacacacactggagggtcaagcagaactccaaaaaaccaggaccccacggcaaaaacacccctcaaagcaaagagttcaaatctcaaaataaagaagcc

SEQ ID NO: 70 - protein, 526 aa, Danio rerio
MFLWSPEASSVPCCKSEFINRALLEMFSGFQERYDHEDELYRDEEDSSGLSDSDSELEFRLHSQLHYNAEFQENHEESSKVQPLVPQTQLQPETMPPAPPVDVIVIDFRADSFTVSDSTEDDESVCANKVQSLKHCKRKSQPSRFRSPSPPQVQAGRSSPDDVVVLDSDLEQSSSESEPPFVEDLDSDTDSDSDSDGLENWMILGKGRQDEDQSIQLNLAQSSFVNTEYVNYRGQEQCGLYQKRINRLRSIIKGTARGMSQTAPHTDTTLKNHHVSQLQNWSSLQKLPHIKEGAVLLIMWASGTLTEDLSKQSLLELFAARPTSDDCLERAFWYKRCHRCGMTGHFIDACPQIWRQYHLTTTAGPIRKSADPKACQKRAYCYNCSRKGHFGHQCSQRRMYNWSYPSLPVITYYDTVNDIKCRDFRLKKKAREMQDAGLISPDGGVVTFTPQPPRKKQKVSHSPHPYSNNTNNHHTPKKRIAHTPKHNPQTNQQNTHWRVKQNSKKPGPHGKNTPQSKEFKSQNKEA

SEQ ID NO: 71 - DNA, 513 bp, Danio rerio
ggaaagttcggaggtcagcgctgcgtcgacgcttgctagcttgttctctgcgctcactcttttataatggcaaaacatcacccagatttgatcttttgcagaaaacaggctggagttgccattggaagactgtgtgaaaaatgtgatggcaaatgcgtgatctgcgactcttatgtcagaccgtgtacactggttcgtatttgcgatgaatgcaactatggctcttaccaaggacgctgcgtgatctgtggaggtcctggagtgtctgatgcctattactgcaaagagtgcaccatacaggagaaagacagagacggctgtcccaagatcgtaaatctgggcagctcaaagactgatctgttctatgagaggaaaaaatacggcttcaagaagaggtgaagaaagaaacggaaggagttttttttttgcaaaaatctttttttttgggcccaattttttattttggttttcaacctaattggaataaaaaaatttttttaaaaaaaaaaaaaa

SEQ ID NO: 72 - protein, 110 aa, Danio rerio
MAKHHPDLIFCRKQAGVAIGRLCEKCDGKCVICDSYVRPCTLVRICDECNYGSYQGRCVICGGPGVSDAYYCKECTIQEKDRDGCPKIVNLGSSKTDLFYERKKYGFKKR

SEQ ID NO: 73 - DNA, 2001 bp, Danio rerio
accacttctgaacgcgacttgcatcctgcagcgatgactctttttcaccatgttgttgatcgaggggctaaaaaaagaggacgaaatggtgagcagcggctgtttcctctgtcgtctctgctggactgctatgaatgcgctcggcgggatgaacacatctcttatggagcctccgctgctgccgtgccgccattcggatgcacattttcctccgctcatggacaacaaaactgtcttgctgtagccaacgaagaagggtttgtgaccatcttcaacactggagaaaaacaaagctctgttctgaaagaatggcaggcacatgataatgcagtttttgatattgcctgggttcctggaaccaactgcttagtaactgcatccggtgaccaaactgctcgtctgtgggatgttattactggtgacctgctgggaacatttaaaggacatcaatgcagtctcaaatcagtagctttttacaagcaagagaaagcagtatttagcactggaggcagagatggaaacataatgatttgggatacaagatgcagtaaaaaagatggtttctacaggcaagtaaagcagatcagtggtgctcatatgaaacctgaaagattcactcctcaaacaaagaagagacgtggtatggcgccccctgtggattcccaacaaggtgtgacagtggttttgttttgcgatgagaccaaactcatctcctcaggggcggttgatgggatcattaaaatgtgggatttgcggaggaactatactgcataccaccagaaccccctgcctctacaggcctatccttacccagggtcctgcacgcgcaaactaggttattctggtctttcactggactacactggatctagactcttctctaactgcacagatgacaatatctacatgtttaatatcagtgggctgaaaaccactccaatcgctgtgttcagtggtcacagtaactcctcattttacgtgaagtccactgtgagtccagacagttacaggtgccgctttacccctcccatcaaacacctcttttgcacctccagccaagctcaccagccccaaaatgccatcatcccttcaacaatggatcagccgcagcagcaaatctcctgttcgaaaagccctcacacctgttctccagggtctgtccttcgaacgccgtgtcaaacgccgactggagacgggcgattctgccagctctggtttgggagaggagatcgacggtgtgtctgaactttatcccaatgtcaagaggagtaggagttcagtaagcacactcaagaaagaggactcgtttggtttggaaagtgagaagaggcaaggctcggatggtgcagaggcctctgggaaggagaacagctctcccaggaggactgattggctttctgtgatcagccagaagtttaaagggtcggctcagcctaagagccccagcagtggcagcagtcagcaggatacaagaacactagaatctccagcagcagtctcacctcgtcccatgaaagttttctctccaccaacaaacaagaaagcatcaccttccaaacctatgaaaaaaatctcaagctactttatgaaaagaacccaagactgacgtgcgtgggtgcatgcctgcatgcctgtgtgcgtgcatgtttaaagagattacaagtgtctttcattctacacatttatggtttaaattcagtttcagcttttcttgtaactgagcttcaaaccaaacctagaaaagatttgcagattctgcctgtatttatgtatgtaatgtgtatttttttatctagttgtgcatttatttttaaatgtgtaaaaatatttttctagaaaagggtcaaatgtatgatcatttacagtcatttgacaccacagttgaattctaaagtttctctgataccttcatgtctgtcagacagtttgaaacactttgctccatttaacatgctttttaaactatattaaattatatttgggtgttaaaaaaaaaaaaaa

SEQ ID NO: 74 - protein, 522 aa, Danio rerio
MTLFHHVVDRGAKKRGRNGEQRLFPLSSLLDCYECARRDEHISYGASAAAVPPFGCTFSSAHGQQNCLAVANEEGFVTIFNTGEKQSSVLKEWQAHDNAVFDIAWVPGTNCLVTASGDQTARLWDVITGDLLGTFKGHQCSLKSVAFYKQEKAVFSTGGRDGNIMIWDTRCSKKDGFYRQVKQISGAHMKPERFTPQTKKRRGMAPPVDSQQGVTVVLFCDETKLISSGAVDGIIKMWDLRRNYTAYHQNPLPLQAYPYPGSCTRKLGYSGLSLDYTGSRLFSNCTDDNIYMFNISGLKTTPIAVFSGHSNSSFYVKSTVSPDSYRCRFTPPIKHLFCTSSQAHQPQNAIIPSTMDQPQQQISCSKSPHTCSPGSVLRTPCQTPTGDGRFCQLWFGRGDRRCVLYPNVKRSRSSVSTLKKEDSFGLESEKRQGSDGAEASGKENSSPRRTDWLSVISQKFKGSAQPKSPSSGSSQQDTRTLESPAAVSPRPMKVFSPPTNKKASPSKPMKKISSYFMKRTQD

SEQ ID NO: 75 - DNA, 1328 bp, Danio rerio
ggaccgtagacctgctagctcttagttttgcttttatttatttctagttaactttgttttagaagttgtctttatttttaacctttaacttatcctcgttttgttttaccaaacactccaagctttacaatgtcgtccactcgctctcccctgaaaaccaagaatgaaaacacaatctctactaaaatgaataacatgtcctttgtggacaaagaaaacacgccacccagtttgagctccaccagaatcctggcgtccaaaacggcgcgcaaaatctttgatgaatcagaggggcagtcgaaagcaaagaaaggagcagtggaggaggagcctcttctgaaggagaacccccatcgctttgtcattttcccaattcagtatcatgacatctggcagatgtacaagaaggcagaggcctctttctggacagctgaggaggttgatctgtccaaagatctgcaacactgggattccctgaaagacgaggagagatatttcatctcccatgttctggctttctttgctgcaagtgatggcattgtcaatgagaacttggtggagcgtttcactcaggaggtccaggtgactgaagcccgctgcttctatggcttccagatagcaatggaaaacatccactcagaaatgtacagcctgttgattgacacctacattaaagattctaaagagagggaatttctgttcaatgccattgagacaatgccatgtgtaaagaagaaggctgactgggcactcaactggattggtgataaaaatgcacgatatggtgagagagtggtggcctttgctgcagtggagggaatcttcttctctgggtcttttgcttctattttctggctaaagaagaggggactcatgcctggactcaccttctccaatgagctgatcagcagagatgagggtcttcattgcgacttcgcctgcctcatgtttaaacacttgatcaacaaaccatcagaagaaaccgtgaagaaaatcatcatgaatgcagttgaaattgagcaggagttcctgactgatgctcttcccgtgaagctcattggcatgaactgtgacctgatgaagcagtacattgagtttgtggctgaccgactgttgctggagctgggatttgacaaggtctatagagtggagaatccttttgacttcatggagaacatttcgttggagggcaagaccaacttcttcgagaagcgagtgggcgagtaccagcgcatgggagtcatgtcgggaactacagataacactttcactctggatgctgatttttaaagactcaatctgagtgcttcaacggacacttaacacaa

SEQ ID NO: 76 - protein, 386 aa, Danio rerio
MSSTRSPLKTKNENTISTKMNNMSFVDKENTPPSLSSTRILASKTARKIFDESEGQSKAKKGAVEEEPLLKENPHRFVIFPIQYHDIWQMYKKAEASFWTAEEVDLSKDLQHWDSLKDEERYFISHVLAFFAASDGIVNENLVERFTQEVQVTEARCFYGFQIAMENIHSEMYSLLIDTYIKDSKEREFLFNAIETMPCVKKKADWALNWIGDKNARYGERVVAFAAVEGIFFSGSFASIFWLKKRGLMPGLTFSNELISRDEGLHCDFACLMFKHLINKPSEETVKKIIMNAVEIEQEFLTDALPVKLIGMNCDLMKQYIEFVADRLLLELGFDKVYRVENPFDFMENISLEGKTNFFEKRVGEYQRMGVMSGTTDNTFTLDADF

SEQ ID NO: 77 - DNA, 1956 bp, Danio rerio
gcgcccagcctacccataacccaccgcgcgcgcctccattcttctctccagtccaacatgtccatgatagacagtccgttaagcgtgctcggccagaggacgactggcgagtccgtgaggtctcaaaacgtcatggctgctgcatccattgctaacatcgtgaagagctctctcggtcccgtcggattggacaagatgttggtggatgacatcggagacgtgaccatcaccaatgacggggccaccatcctgaagctgctggaggtcgaacacccggctgctaaagtcctctgtgagctggcagagctgcaggacaaggaggttggagacggcactacatcagtggtgatcattgcggctgagctgctgaagagtgcggatgagctggtcaaacagaagattcaccccacctccatcatcagcggatacagactggcctgcaaggaggctgtccgctacattaatgagaatctcaccatcggcacagatgacctgggcagagagtgtctcctcaacgccgccaagacctccatgtcctccaagatcatcggagttgatgctgagttcttcgctaatatggtggtcgacgccgcagtggctgtgaagtttgtggacggtaaaggggttgctcggtatcccatcaactctgtcaatgtgctgaaggctcacggacgcagccagaaggagagcttcctggtcaacggatacgcactcaactgcactgtcggctcacaagggatggtaaaacgtgtggccaacgctaagatagcctgtctggacttcagcctgcagaaaaccaagatgaagctgggcgttcaggtggtcatcaatgatccggagaaactcgaccaaatcagacagagggaatcggacatcacaaaggaacgtgttcagaagatcctggcatcaggagcgaatgtggttttgaccacaggaggaatcgacgacatgtgtctgaagtatttcgtggacgtgggggccatggcggtgagaagagtcctgaagaaagacctcaaacgcatcgctaaagctactggagccacggtttgctcttctctgtccaacctggagggcgaggagacattcgagccgtccatgttgggtcaggcagaggaggtggtgcaggagagagtgtgtgacgacgagctcatcctcatcaagaacacaaaggcgcgcacctctgcctccatcatcctgcgcggggccaatgatttcatgtgtgatgagatggagcgctctctgcatgacgccctgtgtgtggtgaagcgagtgctggagtctaaatctgtggtgccaggaggaggcgctgtggaggccgctttgtccatctatctggaaaactacgcaaccagcatgggctcccgtgagcagctggctattgcagagtttgctcgctctctgctggtcattcctaaaactctggccgtgaacgcagcgcaggactccactgacctggtggccaaactcagagcctttcacaatgaggctcaggtcaaccccgatcgcaagaacctcaaatggattggtttggatctggtaaatggaaagccaagggataacaagcaggcaggagtgtacgaacccacgatggtcaaaaccaagagtctgaagttcgccaccgaggcggccattaccatccttcgaatcgatgacctcatcaagctcttccctgaccagaaagaaggcgggccctcctatcaggacgccgtccaatcaggctctctagagggctaaggtctctcattggctgattctggtgtcactccgctcagctttgtatttatatgtggatccaactgatgagctgttagtagagattgtgcacttttttgtttgcgttacttatatttttgctattagcatggctgtctgtcatgtttaatgtaaaaaaagttcctttttctgccatttcaaagaaaataaaccagattggtggtgtggaaaagagaaaaaaaa

SEQ ID NO: 78 - protein, 556 aa, Danio rerio
MIDSPLSVLGQRTTGESVRSQNVMAAASIANIVKSSLGPVGLDKMLVDDIGDVTITNDGATILKLLEVEHPAAKVLCELAELQDKEVGDGTTSVVIIAAELLKSADELVKQKIHPTSIISGYRLACKEAVRYINENLTIGTDDLGRECLLNAAKTSMSSKIIGVDAEFFANMVVDAAVAVKFVDGKGVARYPINSVNVLKAHGRSQKESFLVNGYALNCTVGSQGMVKRVANAKIACLDFSLQKTKMKLGVQVVINDPEKLDQIRQRESDITKERVQKILASGANVVLTTGGIDDMCLKYFVDVGAMAVRRVLKKDLKRIAKATGATVCSSLSNLEGEETFEPSMLGQAEEVVQERVCDDELILIKNTKARTSASIILRGANDFMCDEMERSLHDALCVVKRVLESKSVVPGGGAVEAALSIYLENYATSMGSREQLAIAEFARSLLVIPKTLAVNAAQDSTDLVAKLRAFHNEAQVNPDRKNLKWIGLDLVNGKPRDNKQAGVYEPTMVKTKSLKFATEAAITILRIDDLIKLFPDQKEGGPSYQDAVQSGSLEG

SEQ ID NO: 79 - DNA, 2232 bp, Danio rerio
gatgaacatctggagaaccacagacaagaagaacatctggagaaccacagacaagacaacatagtaactgaagacaaccatctggagaaccacagacaagacaacatagacactgaagatgaacatctggagaaccacagagaagacaacatagacactgaagatgaacatctggagaaccacagacgagaagaacatctggagagcttcaggtcacgcccactgattgtgtaatcaccaccgcccaatggcagctcaaagtgacgttcgcgccgtcagcttcgctgctggccaatcggtgccgcctcgccgctaagccccgcccctgtcctgcagtctgagcgcgcccgcgggagttatgctgccgaggctcacattaaatctgcaagaatgagcgacaaaccctgcgaaccgagctgggagcagatcatcaaccgctggagcttcgatttctacgcgttccaggccttcagcgcgttcaggaatggagattacacggccttcacacacttcatcaacatcatcgagagtgtggtggtcaggccggtggacggacacactgacatgatcccaaagctgcgcctcatgcagtttctgtccaggatcaacaacggggacaagctggatctgaccttcgaggagcccaagacccctctggagtcggctctggacgtgctggagagcatctgcagagacatgagcgtcccgcacacagagcagcagctcatacgccacgccatcagcgagatgctggtgatggtgtgtatcaggagcggtgagttcgagaaggctgaggagatgctcaaacacttcagctcctcgtcagactctgctgggaagaagaagctgctgctgcacctgatccgctgccggcgctcctctcacacagtgctgcagctcagctcctacagccacttcaagcaggacatgctgcagttcgtggagcggctcttcagcgtccccgagcccttcctgctgcagatgctgaggcgctctggatggcccagccagaggccggaggtcaggagatccactcaaaacaaccggagattcacagtgaagcccagcaaacgcacacctccacacagcagcgcagcagaggatcatggggcggagtgtgtgtctgcagtgagtcggcagggtctgcagcaggtgtttgatctgctgagtgagcagtacggcgtgggcatctccttcttccagctgcaggacagtgtggaggcggaggcgcagcagcagcaggagggaggagtggcgggccccgagctgtgcctgacgctgagcgagacccccatgcagactgtgatcctcagcgacacggaggagcaggacgagccgcgcaggtacgccggcatgaccatctcacgcctcgtgcaggaggaggacagccagatcagcgctgaacacacacacatagaggacgaggaggaggaggaggagcagacacacacacaccgcggaggagactgcagtttatcagagccgcagtgttcctccactcagcgctcgactcctgcacgtctctgcaaacgctcaaacacacgtgtgcagcaaagtgaatcagagtctcagagttcaccagctcatcgctcgactcctgcacgtgcttcacatcccagcaaccctcgctccagcacacgcaagaaaacaccgagccggcgcaggattattgagtccagtgactctgagaatgagcagacaactcctgcagccgcaacacacacacctagaggacagcggcagcagcacaggtctatccgtgtgtccagcgggtctgaaccggaggtggacagcacatctcctgcagcacacacacgccgctccacacccaccagcacacacacgcgcaccaccaagaggtctaaatggctggatgtatcggggattcaggataactggagtgatgaggattctctgtttcacacttctacagctccagctaaaaagtacaccaggaagatgtggtccgtgcaggagtcggagtggctgaagcagggtgtggtccgctatggtgtgggacactgggagaggatccgctccgccttccccttcgccggacgcaccgctgtaaacctgaaggaccgctggaggaccatggtcaagctcaagatggtctgatgtgtgtgtttgcgagcgtgcgtgtgtgtgtgtgtatgtttcagcacaatcatgaagttttagactgatgttttataacagttctgttcatattaaatgtttctgatgtttggc

SEQ ID NO: 80 - protein, 575 aa, Danio rerio
MSDKPCEPSWEQIINRWSFDFYAFQAFSAFRNGDYTAFTHFINIIESVVVRPVDGHTDMIPKLRLMQFLSRINNGDKLDLTFEEPKTPLESALDVLESICRDMSVPHTEQQLIRHAISEMLVMVCIRSGEFEKAEEMLKHFSSSSDSAGKKKLLLHLIRCRRSSHTVLQLSSYSHFKQDMLQFVERLFSVPEPFLLQMLRRSGWPSQRPEVRRSTQNNRRFTVKPSKRTPPHSSAAEDHGAECVSAVSRQGLQQVFDLLSEQYGVGISFFQLQDSVEAEAQQQQEGGVAGPELCLTLSETPMQTVILSDTEEQDEPRRYAGMTISRLVQEEDSQISAEHTHIEDEEEEEEQTHTHRGGDCSLSEPQCSSTQRSTPARLCKRSNTRVQQSESESQSSPAHRSTPARASHPSNPRSSTRKKTPSRRRIIESSDSENEQTTPAAATHTPRGQRQQHRSIRVSSGSEPEVDSTSPAAHTRRSTPTSTHTRTTKRSKWLDVSGIQDNWSDEDSLFHTSTAPAKKYTRKMWSVQESEWLKQGVVRYGVGHWERIRSAFPFAGRTAVNLKDRWRTMVKLKMV

SEQ ID NO: 81 - DNA, 4135 bp, Danio rerio
ttttattttcgagttgttgcgtttgttgaatgccctcgtcttgtgtttcattgtttattgacaacaaccggtatttctccgttttctatcacgccaaacgggttaaattattaacccagaaatcattagtcgcgtcttgccgcggttaaaaccgctcgagagcctgggaggacagttttaacatatttgtggttcatattcacgctcacacccgtacgactttcgattaaaacacgacgaaggcatgtgcaaaccgtccaaacaacctgcaggatgaaccgtgtacaagtggattttaaagggttgccagcccacatcttggagaacagcattgcagcagaaagccttcaaaacactaggtcatcagacaatgttctcactccattgacatttccaaaatctaaagtggctctgtgggatccatctgccaatggtgaagttgtgagtctgcatttctcgtattacagaaatccgagactcttcttggtagaaaaggcattacgtttggctcaccgtcatgctagacagaccaacaagcctcgatttttctgttttctgcttgggacacttgcagtagacagtgatgaggaaggagtgactataaccctggatcgttttgatccaggtagagagcagacgggatgtcttgggaaagcaccaactgctctgctcccaggagacatccttgtaccatgtgtattcgaagcccagcatgcagctagcagcactgtacactcgagtgaggatttgaacatctcgtttaagatgcttcaacatttctgctgcagtaaagagttgctggagttgtccaaactcctcaccttgcgtgctcaactcagctgttctgagaacatggacaggctcacctttaacttatcctgggctgctgtgactttggcctgcacgctagatgctgtccctattagggctgtgcccatcatccctaccgcactggccaggaacctcagtagccctgcaggtgtgacccaaaacagtaaacgtgggtttctaaccatggatcagaccaggaagcttctcctcatacttgaatctgatcccaaggcttacactctccctctagttggaatatggttgagtggggttacacacattcataatcccttggtgtgggcgtggtgtttgaggtacctgcacagctcctccctcctggacaaggttttgtcagagggaggcaccttcctggtggtcctgtattccctgactcatcgagatccggagttttaccagtgtaagccaagcactggacagcagcaactgagctttcagctgctcacaagcagagagtcactcacactttataagaatgttgagccttcagaaagacgacctttacaatttgagctcagctctgaaaatcaaaatcaggaaactgtgttgtttgaggaagtgctctcacagtctgtgttaacagggacgactcttggagcagcatctgctgctcctcagaataagctgtctatcagtgatcatgactcaggtgtagaagatgaagacctctctcctcgaccatctccaaaccctcatccctgttagtcagcagactaaaggtgttcatcctttagtaccagagctctccatggttttagatggcagctttttggatgggagtgttgttaatactcaaggctcaactcccctttctcatagtcaatcaaatgttcataggagaaacagtagtcctgccctccagggtctctctgttctgagacctctagtacagggaagcgttactggaccccctccaatccgaagaccactaactcccattctttctcagccgaagaacaagttacatcctaatccatcacaacaaacaccacaacacagtgtgagtcgaaaatccctgccctcaatgagaagatcaagagaaggctcttcagcatcatcagtatcatcatcttcatcctcttcctcaacaaaaaacgcttcacccaatggctcttttcatcaacaaaggcagcgtttatctcagggtttcccaaacaaaccccagctgatttattctggggcctccaacatcaggtcatagcagtgccaaaaagagttcatcagtacctagccagacccctgtccctcatccttcccaacatagaatcttccatagcaccccagctgtaaatccttgcaactgtgcaccaaccaccccagcgttgctcctgtccaggatttcttcaacatccaacatctcccacaaacacaacacgtggtttggttttggagtccaaatatttcagagtccggtgttgggagagagtgcaagcatgtattatcaatctcagtcacaaagcaaagatctgtctgaaaatagagaaattgacgacccaaggttttaccacgagctgttgggtcaggtgcaaagccgtctgcaagactctgtgattgtggaggataaggtagagcaggaccaacagagtctcctgaaaagacaaagtctgtcccctgttggccagcagtcaaggaaaccactgacaacttcttccataccccagactcaaaaaacaaagcagccatctagtccacctaatcaggatcgtgtcttaagtgcaacattaaaacagctacaacagtttggggtgaacatcgacttagactcttctcaggaaaaaacgacacgtgccactgtggagagtgccagtacccttgcctgcataaaccctgaagcagtgattccaagactagctctttctgaaccagtgggtgccagcatttgggggccaagtggcagcgtagaccttagtcttgaggccaatgccattgcacttaagtacctaagcgactcacaactttcaagactctcactgggcagtcagtcctctagcccacattcagaccccagcacaattctcctgagaagaccggctgtggaaaagagcaatgttgcactcagtatcctgtcctccagcaatatgtccctagctacctgtaaatacatgaagaaatatggactgattgaaggagaaatcagcagtgaggaagagcaggaggaccccatccaggtagattctgctcttggatgttcagtacaacatgaaacatctaaaacaatcagccttaggcaagaacgtgaagaacaaaacacagccgttctaaaaaacataacaaacaagccgattgtcaatctccatacatctcccattgactctcaagagcagatactacaagacttgaggcctaaaatgcagctgcttttgcgtggcgggacgaactcagagaaggagaatgccacaaagagagatctgattgaacgcaggtcctcgcttactgaaaaccggagaacacaggaagttgtggaccctcaggggtcagtgggaaatttcctagatctgagtaggttacggcagctccccaagctcttttaaagtgcatcatgggtgttttcctaatcgggctgcttctgtgcatcagaggggtttgagagattaatgcaatcctcaaatgtttgtttacatggcataccatgaatgaattttatttttaagtgcgcttttcatttaagcttttaagtatgttttaatgtctttaactttgtcttgttttgtctaacccaaagggatagtttgcccacaaattaaaatttaccctctttttattcctctttcaaaggttttaaacccttttaagtttctttattctgttgaacacaaaagatgatatttgtaagaaatctgaaaacctgtcattgacttccatagtatttatttttcttactatggtactaaatgtttacaggttttcctttttcttcaaaatcatctttcgtttgtttttcatattttgtgctccactaaagacaaaaaaacctaaaaggtttaaaacttttaattattgtgtgaactatccctttaacaggtgctatttccttttatgtggaaatatttgtcttcacctgatgactccttatgtgtcaataactaagtgccttgtatgaagtgattcacttaaggcagtgttgtggttttgtactatatttaaacacacttgtgttttggcattacttgatgtttctgaaattggtgcaaacatttctacgtttttcaatgacttcatgtacatgtatctgtcttataattaaattacatattcttttacacaaaaaaaaaaaaaaaaaaaaaaa

SEQ ID NO: 82 - protein, 1044 aa, Danio rerio
MNRVQVDFKGLPAHILENSIAAESLQNTRSSDNVLTPLTFPKSKVALWDPSANGEVVSLHFSYYRNPRLFLVEKALRLAHRHARQTNKPRFFCFLLGTLAVDSDEEGVTITLDRFDPGREQTGCLGKAPTALLPGDILVPCVFEAQHAASSTVHSSEDLNISFKMLQHFCCSKELLELSKLLTLRAQLSCSENMDRLTFNLSWAAVTLACTLDAVPIRAVPIIPTALARNLSSPAGVTQNSKRGFLTMDQTRKLLLILESDPKAYTLPLVGIWLSGVTHIHNPLVWAWCLRYLHSSSLLDKVLSEGGTFLVVLYSLTHRDPEFYQCKPSTGQQQLSFQLLTSRESLTLYKNVEPSERRPLQFELSSENQNQETVLFEEVLSQSVLTGTTLGAASAAPQNKLSISDHDSGVEDEDLSPRPSPNPHPCSQQTKGVHPLVPELSMVLDGSFLDGSVVNTQGSTPLSHSQSNVHRRNSSPALQGLSVLRPLVQGSVTGPPPIRRPLTPILSQPKNKLHPNPSQQTPQHSVSRKSLPSMRRSREGSSASSVSSSSSSSSTKNASPNGSFHQQRQRLSQGFPNKPQLIYSGASNIRSFILGPPTSGHSSAKKSSSVPSQTPVPHPSQHRIFHSTPAVNPCNCAPTTPALLLSRISSTSNISHKHNTWFGFGVQIFQSPVLGESASMYYQSQSQSKDLSENREIDDPRFYHELLGQVQSRLQDSVIVEDKVEQDQQSLLKRQSLSPVGQQSRKPLTTSSIPQTQKTKQPSSPPNQDRVLSATLKQLQQFGVNIDLDSSQEKTTRATVESASTLACINPEAVIPRLALSEPVGASIWGPSGSVDLSLEANAIALKYLSDSQLSRLSLGSQSSSPHSDPSTILLRRPAVEKSNVALSILSSSNMSLATCKYMKKYGLIEGEISSEEEQEDPIQVDSALGCSVQHETSKTISLRQEREEQNTAVLKNITNKPIVNLHTSPIDSQEQILQDLRPKMQLLLRGGTNSEKENATKRDLIERRSSLTENRRTQEVVDPQGSVGNFLDLSRLRQLPKLF

SEQ ID NO: 83 - DNA, 773 bp, Danio rerio
ggggacatccggtggttttcgccccgcgctgttccgctgtgaagatgccgaagttttattgcgattactgtgacacatacctgacacatgattcgccctcggtcagaaagacacactgcagtggacgcaaacacaaagaaaatgtgaaggattattatcagaagtggatggaggagcaagctcagtctctcattgacaaaacgacggctgcattccagcagggtaaaattccgcccaccccgttccctggagcccctccacctggcggatccctgctacctcatccaagcattggcggacctccgagaccgggcatgttaccagctccaccgatgggtggtccacctatgatgcccatgatgggcccccctcctcatgccatgatgcctggaggaccaggtccaggtatgcggccaccaatgggtgggcctatgcaaatgatgcctgggccacacatgatgcgacctcctgctcggccaatgatgccagccgttagacctggtatggtgcgtcctgatcgataaaactacagtgccatgacttccaacccgatacccacggaagccaaatgtctaaactggttttacagtcgatttgaagagttttattgcccatttgcgttttatgatgactgatgtctgcggttcaggaaacctcgttccgcttgagtgaccgggattatcacaaattcttgtaaaatttaatgaaccttccattggctttcatttcatatttatgacagaccagagggtgttgcgtaatgggtttgtgta

SEQ ID NO: 84 - protein, 159 aa, Danio rerio
MPKFYCDYCDTYLTHDSPSVRKTHCSGRKHKENVKDYYQKWMEEQAQSLIDKTTAAFQQGKIPPTPFPGAPPPGGSLLPHPSIGGPPRPGMLPAPPMGGPPMMPMMGPPPHAMMPGGPGPGMRPPMGGPMQMMPGPHMMRPPARPMMPAVRPGMVRPDR

SEQ ID NO: 85 - DNA, 1696 bp, Danio rerio
gcggggacagaatccaagatgtcgcttacaagttttcttcctgcgcccacccaactgtcccaggaccagctggaggcagaggagaggtctcaggcatacagatcccagtccactgctttggtctccacccgcagagaccctcctccttacggcttcagaaagtcatgggtaccccgggctctagaggactttggagatggaggagcttttccagagatccatgttgcccagtatcctctggaaatgggtaggaagaaaaggacgtccaatgctctggcagtgcaggtggatgcagagggcaagatcaaatatgatgctattgctagacaaggccaaagcaaagacaaggtcatatttagtaaatacacagacatgcttcctaaggaagtgctaaaccaagatgatccagagctgcagaaaccagatgaagaggcagtgagagagcttacagaaaagaccagattagctctggagaaacaggtttcacagaaaatcgcggcagccatgcctgtgcgtgctgcagataaacaggcccccgcgcaatacattcggtacacaccctctcagcaaggtctcgcatttaactctggagcaaaacagagggtcatccgtatggtggaaatgcagaaagatccaatggaaccccctcgattcaaaatcaataagaaaattcctcgtggacctccttcccctcctgctccagtcatgcactctccaagcagaaagatgacagtgaaagaacagcaggaatggaagattcctccttgtatttccaactggaagaacgcaaagggttacaccattcctctagacaagcgtttggcggctgacggacgaggcctccagaccgtccacatcaatgaaaactttgccaaactggcagaggctttgtacatagcagacagaaaggccagagaggctgtggagatgagagctcaagtggaaaagaagatggcccagaaggaaaaagaaaagaaagaggagaagctgagagagctggctaagatggccagagacaggagagctggtatcaaaccccacggtgataaaggtggtgaagactccgaagtcagggagcgtgatgagattcgtcacgataggaggaaagataggcagcatgacaggaacatctccagagccgcccccgataagaggtcgaagcttcagagggaccaagacagggacatcagtgagctcattgctctcggccagccgaacccgcgcacctctagtgaggctcagtatgaccagagactcttcaatcagagcaagggaatggacagcggttttgctgggggagaggatgagatgtataatgtgtacgatcagcccttcagaggtggcagagacatggcacagaatatttacaggcccagtaagaatgtggataaagacatgtatggagatgacctggacacactcatgcagaacaacaggtttgttcccgaccgagacttctcaggtgctgaccatggcccccgccgggatggtcctgtacagtttgaggaggatcctttcggtctggacaagttcttggaggaagccaagcagcacggaggctccaaaagagcgtccactagtgggcgctccaaggactatgaccatgagaagaaacgcaggaaggagtgagacactttctctgtcaaacttcaggcttaaatccttcttcccatacttttttttttgtaactgaata

SEQ ID NO: 86 - protein, 536 aa, Danio rerio
MSLTSFLPAPTQLSQDQLEAEERSQAYRSQSTALVSTRRDPPPYGFRKSWVPRALEDFGDGGAFPEIHVAQYPLEMGRKKRTSNALAVQVDAEGKIKYDAIARQGQSKDKVIFSKYTDMLPKEVLNQDDPELQKPDEEAVRELTEKTRLALEKQVSQKIAAAMPVRAADKQAPAQYIRYTPSQQGLAFNSGAKQRVIRMVEMQKDPMEPPRFKINKKIPRGPPSPPAPVMHSPSRKMTVKEQQEWKIPPCISNWKNAKGYTIPLDKRLAADGRGLQTVHINENFAKLAEALYIADRKAREAVEMRAQVEKKMAQKEKEKKEEKLRELAKMARDRRAGIKPHGDKGGEDSEVRERDEIRHDRRKDRQHDRNISRAAPDKRSKLQRDQDRDISELIALGQPNPRTSSEAQYDQRLFNQSKGMDSGFAGGEDEMYNVYDQPFRGGRDMAQNIYRPSKNVDKDMYGDDLDTLMQNNRFVPDRDFSGADHGPRRDGPVQFEEDPFGLDKFLEEAKQHGGSKRASTSGRSKDYDHEKKRRKE

SEQ ID NO: 87 - DNA, 2301 bp, Danio rerio
ttctggcaacatggcgacctgcttatgagccacgctggtgctgaagttatcgaagccgtcaaaataaacattcttttctgacacttagtattagttaaatagcaaaaatgtcgggtcgtcacaataacaaattaccgacgaacctcccgcagttgcaaaatctgatcaagagggatccgaagtcctacaccgaggagtttttgcagcagtatcgtcattatcagtcaaatgtggagatctttaaacaccagccggataaagccaacaaagacctgtccgagctggtcatgtttctggctcaggttggacactgttatttagaggagctgtcagatttcccccagcagctgacggatctgcttttaaactatcatacagtgctggagtctgatctcagaatgacgttttgcaaagctctgatcatgttgaggaacaaagatctggtcagtcccaccagtctcctggggcttttcttcgagctgctgcgctgtcacgacaaacttctgagaaagactttatatacccatattgtgacggatatcaaaaacatcaacgcaaagcacaaaaacaacaaaatgaaaacgactttgcagaacttcatgtacaccatgctgagggattcaaaccccattgctgccaaaatctctctagatgtgatggttgagctttacagaagaaacatctggaatgatgctaaaactgtgaacgtcatcaccacagcgtgtttctccaaagtcacaaagatccttgtagctggactgaagttcttcctgggaaaagatgaggatgaaaagaaagacgatgattcagaatctgaggacgagggttcaacagccagagatctaatgatgcgatactcaactggaaagaaaacttccaagaacaagaagaaactggagaaagctatgaaagtcctgaagaaacacaaaaagaaaaaaaaagtggaggtctttaatttctctgccatacacttgatccatgatccaaaagacttttcggagaagctcctgaagcagctggagagctcaaacgagcgttttgaggtgaagatcatgatgatggagctcatctctagacttgtgggaattcatgagcttttcctctttaacttctatccgtttgtccagcgtttccttcagcctcaccagagagaggtcacaaaaattttgttgtgtgccgcccagtcatcccatcagcttgtcccgcctgagatcattgagccagtcatcatgaccattgccaataactttgttacagacagaaattcaggggaggtcatgacagttggaatcaacgccataaaggaagtggttgcccgttgtcctctttctatgtcagaagatctgcttcaggatctcactcagtacaaatcccacaaagataaaaatgttgtgatgtctgctaggggtcttatccagctcttcagagacctcaatccaaagatgctaaccagaaaagacaggggaagacccacagaatcctctaaagaggctaaaatccacaaatatggagaactggaggctaaagactacattcctggagctgaagtgctagaagcagagaaagcggaggatgaggaagatgaagatgggtgggagagcgccagcatgagtgatgatgatgaagatggagaatgggtgaatgtgcatcactcttctgatgaagaccaggcagaagtggctgagaaattacagagcatcccggaggaagagcgcaaagctaaagctgccatggtgagcactagtcgactccttacgcaggatgactttaagaagatccgcgtggctcagatggcaaaagaggtgggcaatgcgcccgggaaaggtcagaagaggaagaacgtagacagcgatgaagaagagagaggagagttactcagtctaagagatattgagcgccttcacaagaaacccaaatctgacaaagaaacacgattggcaactgccatggctggacggactgacaggaaggagtttaccaaaaagagaggtaaactaaacccgtatgccagcaccagtaacaaggagaagaaacggaagaagaacttcatgatgatgagacacagtcagaatgttagaactaaaggcaaacgctccttcagagagaaacagatcgcattaagagattcactcttgaagaagagaaagttcaagtagttcccacttttaatggactggaactaatttatgcttcgtgatctgtgtggaccattgtgtaaaggaagacagtgaacagacttggctgtgtgggttttcttgttgccttttaataaaaacatttaaacagttaaaaaaaaaaaaa

SEQ ID NO: 88 - protein, 682 aa, Danio rerio
MSGRHNNKLPTNLPQLQNLIKRDPKSYTEEFLQQYRHYQSNVEIFKHQPDKANKDLSELVMFLAQVGHCYLEELSDFPQQLTDLLLNYHTVLESDLRMTFCKALIMLRNKDLVSPTSLLGLFFELLRCHDKLLRKTLYTHIVTDIKNINAKHKNNKMKTTLQNFMYTMLRDSNPIAAKISLDVMVELYRRNIWNDAKTVNVITTACFSKVTKILVAGLKFFLGKDEDEKKDDDSESEDEGSTARDLMMRYSTGKKTSKNKKKLEKAMKVLKKHKKKKKVEVFNFSAIHLIHDPKDFSEKLLKQLESSNERFEVKIMMMELISRLVGIHELFLFNFYPFVQRFLQPHQREVTKILLCAAQSSHQLVPPEIIEPVIMTIANNFVTDRNSGEVMTVGINAIKEVVARCPLSMSEDLLQDLTQYKSHKDKNVVMSARGLIQLFRDLNPKMLTRKDRGRPTESSKEAKIHKYGELEAKDYIPGAEVLEAEKAEDEEDEDGWESASMSDDDEDGEWVNVHHSSDEDQAEVAEKLQSIPEEERKAKAAMVSTSRLLTQDDFKKIRVAQMAKEVGNAPGKGQKRKNVDSDEEERGELLSLRDIERLHKKPKSDKETRLATAMAGRTDRKEFTKKRGKLNPYASTSNKEKKRKKNFMMMRHSQNVRTKGKRSFREKQIALRDSLLKKRKFK

SEQ ID NO: 89 - DNA, 1630 bp, Danio rerio
gaattcggcacgaggtccggtaccggtgatctaaccacgccgcaaatatgatgggccgaccggttctcgtgcttagtcagaacatcaagagagaatctggacggaaggtccagattggaaacatctctgcggctaagaccatagcagacatcatcaggacatgcctaggaccaagggctatgatgaagatgctgctggatcccatgggtggtatcgtcatgaccaatgatggcaatgccattttgagagagatccaggtccagcatcctgctgccaagtccatgattgagattagccgcacacaggatgaagaagtcggagatggtaccacctctgtcatcattcttgcgggtgagatgctgtcagtggctgagcagtttttggagcagcagatgcatcctactgtggtgatcggtgcatacagacaagctctggatgacatgttgaacatactcaaagacataagcactccagtagatgtgagtaatcgagacatgatgttgaagataataaactctgcaatcaacaccaaagcactcagccgctggtccacattggcctgtaacatcgctcttgatgctgtccgtactgttgagctggacgagaatggacgcgaagagattgacattaagaagtatgccaaagttgaaaaggttcctggtggcatcatcgaagactcatgcgtgctgaggggtgtgatggtgaataaggatgtaactcatccccgcatgcgcagactcatcaagaaccccagaattgttctgctggactgttctctggagtacaagaagggcgagagccagactgatattgagattgctcgtgaggaagacttcgcccgcattctgcagatggaggaagaatacgttcagcagatctgtgaagacatcattcggctcaaacctgacctaatcttcacagagaagggcatttctgatctggctcagcactatctgatgaaagcaaacatcactgctatccgccgtatcaggaagacggacaacaaccgcattgcaagagcgtgtggtgcacgtattgccagcagaaccgatgaactcactgaaaatgatgtgggtacaggaacaggcctttttgaagtcaagaagatcggcgacgagtacttcacctttgtgacagagtgtaaagaccccaaagcatgtaccatcctgctcagaggagccagcaaggaaatcttggcggaagtggaacgaaacctacaggatgccatgcaagtgtgtcgtaatgtcctgctggacccatatctgctgccgggtggtggcgcagtggagatggaggtgtcccatcggctgacggagcgctctcgagctatgactggtgtggagcagtggccgtaccgcgctgtagctcaggctctagaggttgtccctcgcacactcatccagaactgtggagcttcagccattcgtgtgctcacttctctgagggccaagcacactcaggagggaaactcctcatggggtgttaatggagagacaggaactcttgctgatatggagcagctgggaatctgggaaccgctggctgttaaagcccaaacttacaaaactgcagtggagacggctatcctgttactgcgcattgatgacatcgtttctggccacaagaagaa

SEQ ID NO: 90 - protein, 526 aa, Danio rerio
MGRPVLVLSQNIKRESGRKVQIGNISAAKTIADIIRTCLGPRAMMKMLLDPMGGIVMTNDGNAILREIQVQHPAAKSMIEISRTQDEEVGDGTTSVIILAGEMLSVAEQFLEQQMHPTVVIGAYRQALDDMLNILKDISTPVDVSNRDMMLKIINSAINTKALSRWSTLACNIALDAVRTVELDENGREEIDIKKYAKVEKVPGGIIEDSCVLRGVMVNKDVTHPRMRRLIKNPRIVLLDCSLEYKKGESQTDIEIAREEDFARILQMEEEYVQQICEDIIRLKPDLIFTEKGISDLAQHYLMKANITAIRRIRKTDNNRIARACGARIASRTDELTENDVGTGTGLFEVKKIGDEYFTFVTECKDPKACTILLRGASKEILAEVERNLQDAMQVCRNVLLDPYLLPGGGAVEMEVSHRLTERSRAMTGVEQWPYRAVAQALEVVPRTLIQNCGASAIRVLTSLRAKHTQEGNSSWGVNGETGTLADMEQLGIWEPLAVKAQTYKTAVETAILLLRIDDIVSGHKK

SEQ ID NO: 91 - DNA, 630 bp, Danio rerio
gttttacgctacaattttttctcatctaggtggaggattttctctcaaagtccaccgccatcatgaaactcgtcaggtttttgatgaaattgagccatgaaaccgtcaccattgagctgaagaatggcacacaggtccatggcacgattacaggtgtggacgtcagcatgaacacacacctcaaagctgtgaagatgaccctgaaaaacagagagcccactcagctggagtcgctcagcatccgaggaaacaacatccgctacttcatcctgccggacagtcttcctctggacacgctgctggtggacatcgagcccaaagtcaagtccaagaagagagaagctgtggctggccgtggtcgaggaagaggacgtggtcgtggccgaggaagaggacggggccgcggtgggccgaggagatgaagatctcaagccactgtacttttgtgcgcgttgtacagcattttttgttcttcgtataggagatattttgcgttttgtacattaaattctgttcttttgacaactgtttcgagtcttttgttttgtggtgatcatatgagggatgattctgcaagggtgtctggttatttttctacattcttaccaataaaaactgcttgattgaaaa

SEQ ID NO: 92 - protein, 119 aa, Danio rerio
MKLVRFLMKLSHETVTIELKNGTQVHGTITGVDVSMNTHLKAVKMTLKNREPTQLESLSIRGNNIRYFILPDSLPLDTLLVDIEPKVKSKKREAVAGRGRGRGRGRGRGRGRGRGGPRR

SEQ ID NO: 93 - DNA, 1773 bp, Danio rerio
ggaagtgctaatttggcgggagcagttgaagatggatgcgcgcagagtgaagcagaaagtgaacgcgggctttaaaatgcgtggattaatcctgcgacccgagtccagccggttcctgctgcaggttctggagtccgtcagtccggcagagctggaggaggttctggagcgggttctagactctgtggagaaacagccactgtcctccagtatggtggatctgtgtgtgctggaagccgctgtgcaggactgcagtcagtcgtgtgacgagaccattgatcatgtcttcaacatcattggagcgtttgatgttcctcgcttcatcttcagcacagagaggaagaagtttgtgccgatcagcatgaccaatcacccgctgcctaaactctgcggtcaggcccgagataaagctgagctgttcagagagagatacaccatcctgcagcagcgaacgcatcggcacgagctcttcactccgcctgtcatcggttcagctcaggatgaaggaagaaacaagttccagctgaagacagtggaggctcttttgtgcagcagcgctcggctcggggaggtgattgttctgggcatgatcacgcagctccgagagggtaagttctacctggaggaccccagtggtactgtgcagctcgacatctcaaaggcacagttccacagtgggctgtacactgagagctgcttcgtgctagcggagggctggtatgaggactctgtgttccatgtcagtgcgttcggcttcccgcctacagaaccctcgtccttcaccagggcgtatttcggcaatgtgaacttcttcggcgggccgtccagcacagcggtgaaagcgtccgctaaactgaagcagctggaggaggagaacgaggacgccatgtttgtgctggtgtcagacgtgtggctggacagtgtggaggtgctggagaagatacacaccatgttctctggatactccgctctgccgcccacctgcttcatcttctgtgggagtttctcctcggctccgtatggcaggactcagctgcgctcgctcagagactcctttaaagcgctcgctgacctcatctgtgagtttcccagcattcacagcagcagtcggtttgtgtttgttcctggtccagaagaccctggacccggcgcggttttacccaggcctccactggcggagcacatcacagaggagtttcagcagcgtgtgccgctctctgtcttcaccaccaacccctgcaggatccagtactgcagtcaggagctggtgattattcgggaggatctggtcaataagatgtgccggaactgtgagcgtctgccgagcagcagcctggacatccctacacactttgtgaagaccgtgctgtctcagggtcacctgacgccgctgccgctgtacgtgtgtccggtgttctgggcgtacgatcacacactccgcctgtatcctgtaccagacgtcatcgtgtttgcagacaaatacgacccgttcaacatctgcaacaccgactgcctgtgcatcaacccgggctccttcccgagaagcggattctccttcaaggtgtattacccgtccagcaggactgtggaggacagtaagctccagggcctgtgacccagtggaacgcccccccgcctcatttctgttgccctctgtaaatgtggatgttttaggttgtgtttcttcccgtgacgtttgattttatgcccttttaaagtccaataaaaaaataaaaatttgagacgtaaaaaaaaaaaaaaaaaaaaaaaaaa

SEQ ID NO: 94 - protein, 527 aa, Danio rerio
MDARRVKQKVNAGFKMRGLILRPESSRFLLQVLESVSPAELEEVLERVLDSVEKQPLSSSMVDLCVLEAAVQDCSQSCDETIDHVFNIIGAFDVPRFIFSTERKKFVPISMTNHPLPKLCGQARDKAELFRERYTILQQRTHRHELFTPPVIGSAQDEGRNKFQLKTVEALLCSSARLGEVIVLGMITQLREGKFYLEDPSGTVQLDISKAQFHSGLYTESCFVLAEGWYEDSVFHVSAFGFPPTEPSSFTRAYFGNVNFFGGPSSTAVKASAKLKQLEEENEDAMFVLVSDVWLDSVEVLEKIHTMFSGYSALPPTCFIFCGSFSSAPYGRTQLRSLRDSFKALADLICEFPSIHSSSRFVFVPGPEDPGPGAVLPRPPLAEHITEEFQQRVPLSVFTTNPCRIQYCSQELVIIREDLVNKMCRNCERLPSSSLDIPTHFVKTVLSQGHLTPLPLYVCPVFWAYDHTLRLYPVPDVIVFADKYDPFNICNTDCLCINPGSFPRSGFSFKVYYPSSRTVEDSKLQGL

SEQ ID NO: 95 - DNA, 2646 bp, Danio rerio
cgggggcagacggacgcacgcggaacggtaagatggcggcggacggaggccgagctgcgttaccctgcgccttcagccctaaatcacatcagtttttggctctctgcgcgcaagatgggagactgcgaatctggaataccgagagcaaaaccctacaacaggaatacgtgccttccgcgcatctgagcgcagcctgtacgtgtgtgacatggggtccgtgccgagcagttcaggaggtgcctcagaggaagaggaggaaatgtgatgcggcttcatcagctgagcagtcagatctgttggcactgggcactgctgctggcaccatcctcatctacagcacactgaagggagacctgcattgtacattggatggaggacacagtgggcctgtgaacagcgtccagtggcatccagaagactctgtgttatacagcggttcagatgacacacacatagccgagtgggacttgaaaacggggaaagtttgctgcaaatggaaggcagacagatccgctatcagcagcctgtgtataagtccagatggaaagatgctgctctctgcaggaatgaccatcaagatgtgggacctggagactaaggaagtgtacaggaaattcacaggtcactctaccatggttacgactctatgctttgccacaacacgacctccagacagcaatgggatgtatttcctttcaggagccgtacatgacagactcctgagtgtgtggcaggtacggtccgatggcaaagacaagaactctgtggtgtcttttactctgacagatgaaccccaacacatagaccttcagacatccaacagcaaagatgaggctgtgcggttagcagtggtttgtaaggatgggcagcttcatctttttgagcactttttgaatgggcagtgtaagaagccactgtctccagcgtgttcccttcaggtgtccactatgaagggagagtctccagtgcctgtgcctctactggcagctgctctgtgtgcggacagacagaacctgatgctggcatatggacatcacctgcagcccgtcatggagaaaacaccttttaacacatcaaaaaagcacacatgcttgaacccgagatgtacatgccacactttctctttctgtagacacgactgtctccaaggtgaagacaccagttgtaaatcagcaaagcaaagttttaatcccaggagttccaggacataaggcaccaattaaattaactcccaatgaaggagagaaaaggaaaaaaggggccagtactacagaggtgtctatcgaggaacggttggagcagattgagttgagtgctggaagaaaaggtgctgataaaggctcgtcctccctgcagacagacagtttcgctgtgctgcttgtccagggcttagagagcaaagatgagaagatccttaataaaatatttcaaaccaaaaaagagacgttgatcaagaacactgttgctcggttaccacctcctgctgtcctgccattgctggaagaactctccagaagactgcagggacatccatacgcggctttgcttatagttcattggtttaaggctgtacttacgcaacacacatcatacttgtcttcattgccagatctagtgtctcagttgggctcaatctaccaaatgatcgagagcagggtgaagttatatcatcagatcaccagactccatggaaaactctacctgctcatgacacaggtggcgacaagcaacagtgcaacaaaagttggtgaaattgaccacacagcaaagctggtatatgaagaagagtcttcagaggatgaagattcaggagatgaaggtcgtcctgaggatgactctgataactgggaggaggatgaggaggctatggaggtgactggagacaaaaagaaccacaaagatgaggaagaccaagacatgaatgaggaatcaaaagcaaatggagactcagatctggaccctgaaaatgagagtgaggaggaatagtactcaatcaggctgcaggattcagataaaaggacccttacatttatatatatgattttgttttacttttcattttattgtaacaaaacaaaaaggcgaagttttatagagtttcgataatcgttgcggcctggatctgtggttgtccttttttgaactgttatgaccagactgacagcccaaacaaacacattcgaggtccatttcagtcgcttttcccttgatttggaaacggcctctgttttgttgcccgggttttatgggattctctggggttcccaaaatctccatctccctgcaagatacattcttaatgggcgtcccatgtccataagatcgatcccattatatgacatgatcatgattctgctcatcatcagtgtccatctttaatttatctgtggtcattacactcgctgtcacagtcatttctcatgtctgacttctgtgtcctgtgtgtaatttgagaagaagattattacaccctctgcagtcatgactgttctcagttttgaggacaaaacaatgtgaagctttcccagaaaaaacgaatatcctaaaaacagtcacagaggtgtgtatgataactatacagtggttacagccaaccagaacttttcatgcaaacaatatatcaacaactgttgtacagtttaaatg

SEQ ID NO: 96 - protein, 650 aa, Danio rerio
MAADGGRAALPCAFSPKSHQFLALCAQDGRLRIWNTESKTLQQEYVPSAHLSAACTCVTWGPCRAVQEVPQRKRRKCDAASSAEQSDLLALGTAAGTILIYSTLKGDLHCTLDGGHSGPVNSVQWHPEDSVLYSGSDDTHIAEWDLKTGKVCCKWKADRSAISSLCISPDGKMLLSAGMTIKMWDLETKEVYRKFTGHSTMVTTLCFATTRPPDSNGMYFLSGAVHDRLLSVWQVRSDGKDKNSVVSFTLTDEPQHIDLQTSNSKDEAVRLAVVCKDGQLHLFEHFLNGQCKKPLSPACSLQVSTMKGESPVPVPLLAAALCADRQNLMLAYGHHLQPVMEKTPFNTSKKHTCLNPRCTCHTFSFCRHDCLQGEDTSCKSAKQSFNPRSSRTKAPIKLTPNEGEKRKKGASTTEVSIEERLEQIELSAGRKGADKGSSSLQTDSFAVLLVQGLESKDEKILNKIFQTKKETLIKNTVARLPPPAVLPLLEELSRRLQGHPYAALLIVHWFKAVLTQHTSYLSSLPDLVSQLGSIYQMIESRVKLYHQITRLHGKLYLLMTQVATSNSATKVGEIDHTAKLVYEEESSEDEDSGDEGRPEDDSDNWEEDEEAMEVTGDKKNHKDEEDQDMNEESKANGDSDLDPENESEEE

SEQ ID NO: 97 - DNA, 1197 bp, Danio rerio
aaacacacacgcacggggggaacattggattaaacagccgccatgcagaataaagaaaaccgggaacccagagttcaacaaacaccgtctgctggtgtgggtccgctcagagttgagatgaatccagacacacacgcagtctcaggtcctggcagagttcctgtcaagtcaaactccaaagtgctgtccatcgatgactttgacattggccgtcctctgggaaagggtaaatttgggaacgtgtatctggcgcgcgagcggaagctgaaggtggtgatcgcgctgaaggtgctcttcaagtctcagatggtaaaagagggagtggagcatcagctgcgcagggagatcgagatacagtcacacctcaggcatcccaacatccttcgcttctacaactacttccacgacgacacccgtgtgtttctgattttggagtatgcgccgcgtggtgagatgtacaaagagcttcagcgatacggacactttgacgatcagcgcactgctacttacatggaggaggtgtcggacgcgctgcagtactgccatgagaagaaggtgatccacagagacattaaaccagaaaacctgctgctgggatacagaggagaactgaagatcgcagacttcggctggtctgtgcacgcgccctcactcagacggcggacgatgtgcggtacgctggattacctgccgccagagatgatcgagggccacagtcacgatgagaaggtggatctgtggtccatcggtgtgctgtgctacgagtgtttagtgggaaatcctccattcgagactcgccagcacgccgagacatacaagcgcatcactaaggtggatctgcagttcccgaagctggtgtctgagggcgcgcgggacctgatctccaagctgctgcgccacagtccctccatgcgcctgccgctgcgcagtgtgatggagcaccgtggggtgaaggccaactcccgcagggtgctcccgcccgtctgcagctccgacccacactgaccccccaggacctgattgaggcctggcatatacacccacccgactcaatctgtgtgtgtgtgtgtgtgtgcgtcagagcaaagcatctccactgtatcacacctgttcttctgttcacatccatgttttaatcttgtctgtcttgtttaatcctgtcaaatatataaagctgtttaaataaaaaaaaaaaaa

SEQ ID NO: 98 - protein, 320 aa, Danio rerio
MQNKENREPRVQQTPSAGVGPLRVEMNPDTHAVSGPGRVPVKSNSKVLSIDDFDIGRPLGKGKFGNVYLARERKLKVVIALKVLFKSQMVKEGVEHQLRREIEIQSHLRHPNILRFYNYFHDDTRVFLILEYAPRGEMYKELQRYGHFDDQRTATYMEEVSDALQYCHEKKVIHRDIKPENLLLGYRGELKIADFGWSVHAPSLRRRTMCGTLDYLPPEMIEGHSHDEKVDLWSIGVLCYECLVGNPPFETRQHAETYKRITKVDLQFPKLVSEGARDLISKLLRHSPSMRLPLRSVMEHRGVKANSRRVLPPVCSSDPH

SEQ ID NO: 99 - DNA, 2007 bp, Danio rerio
tggctgccatctgtaacttccatgtgctgttttattatatcacaaaataactgctctcatcatttttcttatccgtaaagttaatttcattgtattggattaacactgacatttacgtacagagagccggttgttcaaagaagccgtcagaatgcaacacgacgacgttatatgggaccttatcggcaataagagtttttgttcctataaagttaaaacgaaaactcagcagttctggcgaaatgaatacaacatcacaggcctttgcaacagatcatcatgccctcttgctaatagccagtatgcaactataagagaagaaaaaggccagtgttttctctacatgaaagttattgagagagcagcatttccttctcgcatgtgggagaaggtgaaacttgacagaaactatgcaaaggctctggaacagatcgacgagaacctcatctactggccacggtttatccgtcacaagtgtaaacagcgattgaccaagatcacccagtatctcattcgaatccgcaagctcacgctaaaacgacagagaaaactggttccactcagtagaaaagtagagagacgagaaaagaggagagaggaaaaagctcttattgccgctcagctggaaaatgccatcgaaaaagagctgctagatcggctaaaacagggaacatatggagacatttacaacttccccatcaatgccttcgacaaagctatggaaaaacaggatgaagaaagtgaatctgaggaggaggaagaagaggaagacgaagaggagtctggaacgagagaatttattgctgaggatgagtttgaagagagtgatctcagtgattttgaggaattgaacaacctgaaggacagcagtgacgaagaagtgaacgatgaagaggaatccagtgaggagtcagaggaagagtctgaggaggaggaggtgaagagcaaggccaaatctaaaggaaaagctccactcaaaggcccaagcaggaaaaacgagcctatgtggagatcgagtatgaacaggagacagaaccagctcagaaaagcaaggccacatagaggctctgtggaacgtttccaaatcatggaatgtgatcatttaccagacacttggattcagttagaacgtacactggtgtgttcaaacactcccattaaaagctttcttgtgggaatcttttttttttttttttttttacattgaattatgctgatgccaagcataagtgctgttttaagtatacatttggattaacttgatccatgtaaaaaataataaaattaagttcatatgacatgcagggtttaccaagtagtggctggtgttgtaaaagggtgaacatgttaaatatgttttgtatatgaatacatggatttgtattgacgttaacagttgataatcaagctgcaaggagtaaaattatctttagtaaaaatagttattgaaatgaatctatatacacactagtatcaatgcagagataaatgggatagaggaacggtaaatgttttatttttggtcggatgtctttttaatcaggagcctactgtcttaaccatagcttacctcatttaataataatgtaaacaacaacaaaggtcatattttaggttttatgcttattaaattgattggaagaatctcaagcaatttaaacaaaaaaaaacaaatcaggtttatcaactttatttaaaaaacaacaacaaccctgccacgtttagtacctgcattcatagaatggagtaaatagattcccgttctatcaagccaacttcacttcattcgagaggaaaagtgacatatggtcaaagcttacggacaagatgctgcaaggacattttggatacacttgaagactgggtcagggacacaactgctgtctacctctcttagaagagcctggggggggggaaactggaaatgttataatggcgttaaacagtgtgcagaaacaagatcattttacagtcattgaataaaacactggcttgtaaatctgaaaa

SEQ ID NO: 100 - protein, 351 aa, Danio rerio
MQHDDVIWDLIGNKSFCSYKVKTKTQQFWRNEYNITGLCNRSSCPLANSQYATIREEKGQCFLYMKVIERAAFPSRMWEKVKLDRNYAKALEQIDENLIYWPRFIRHKCKQRLTKITQYLIRIRKLTLKRQRKLVPLSRKVERREKRREEKALIAAQLENAIEKELLDRLKQGTYGDIYNFPINAFDKAMEKQDEESESEEEEEEEDEEESGTREFIAEDEFEESDLSDFEELNNLKDSSDEEVNDEEESSEESEEESEEEEVKSKAKSKGKAPLKGPSRKNEPMWRSSMNRRQNQLRKARPHRGSVERFQIMECDHLPDTWIQLERTLVCSNTPIKSFLVGIFFFFFFLH

SEQ ID NO: 101 - DNA, 1247 bp, Danio rerio
acacacgccggggttcatcatggcggcgggaccgatttcagaacgaaatcaagatgccgctgtatatgtgggtggtctcgatgagaaagtctctgagcctcttttgtgggagcttttccttcaagctggtcctgttgtaaacacacacatgcccaaagacagagtaacaggacaacaccagggttatggctttgtggagtttctcagcgaagaagatgcggactacgccataaaaataatgaatatgatcaaactttacggcaagcccatacgagtcaacaaagcttcagcgcacaacaagaacctcgacgttggtgccaacatcttcattggcaacttggaccctgagatcgacgagaaattactttatgacacattcagcgctttcggtgtgatccttcagacccccaagatcatgcgagatcctgatacgggcaactcgaaaggttacgccttcatcaactttgccagttttgatgcgtcagatgcggctatcgaggctatgaatggccagtatctgtgtaatcggcccatcactgtttcatacgcgtttaagaaagactcaaagggtgaaagacacgggtcagcagccgagcgtctccttgccgcacagaatccactgtcccaggccgatcgcccacatcagcttttcgcagatgctccacctcctccaagcatgcccacgcctgtcatgactgccctcggagcagcaatgcctattccaggaatgcctcctcccggtgctttcccacctgttcctcctcctggcacaatgccacctgggatgcctcccggtatgcccatgcctccagcaccaggtactcctgcgccacagggcggtggaggacccccaccgggacacccgcccttccctcctgctggaatgcatccgccaggaatgccgcatatgccgatgcctccaacgggacctccaggaatggtgcctcccccacccggcccaccaggatctaaccaaccaagagcaccgcctccaccacaaatgcctcctccacccatgggtgtccctcccagaggaccctttggacccccaatgggtccaccaatgcatccgggtatgaggggtcctcctccaccaatgccgcctccaggttatggtgcaggaccacccaaacccccaccctttggttttcaaaaagggcctcctatgccgctcgtcctcctgacccaaaggtccgatgaaagcaccggcccttcctaaaccccaatgtgaaaaccaaactttgtct

SEQ ID NO: 102 - protein, 399 aa, Danio rerio
MAAGPISERNQDAAVYVGGLDEKVSEPLLWELFLQAGPVVNTHMPKDRVTGQHQGYGFVEFLSEEDADYAIKIMNMIKLYGKPIRVNKASAHNKNLDVGANIFIGNLDPEIDEKLLYDTFSAFGVILQTPKIMRDPDTGNSKGYAFINFASFDASDAAIEAMNGQYLCNRPITVSYAFKKDSKGERHGSAAERLLAAQNPLSQADRPHQLFADAPPPPSMPTPVMTALGAAMPIPGMPPPGAFPPVPPPGTMPPGMPPGMPMPPAPGTPAPQGGGGPPPGHPPFPPAGMHPPGMPHMPMPPTGPPGMVPPPPGPPGSNQPRAPPPPQMPPPPMGVPPRGPFGPPMGPPMHPGMRGPPPPMPPPGYGAGPPKPPPFGFQKGPPMPLVLLTQRSDESTGPS

SEQ ID NO: 103 - DNA, 838 bp, Danio rerio
cggcacgagctcgagtgtctgtgcttcagtcttcgggcggagaattccctctgtaatctagagtcttcctgcgcttcatctccgggataaagatggccccgaaggattatacggcggagaaagagaaatgtaagcggttcctgcaggagttctacactgaggatgactccgggaagaagatcttcaagtatggtgctcagctggtatctctggcacatcgggagcaggtggcgctgctggtggatctggacgacgtggcggaagaagacccggatctggtggagagtgtgtgtgagaacgccaaacgatacacagcgctgtttgcagacgccattcatgagctgctgccggagtacagggagcgggaggcggtggtgaaggacgcgctggacgtctacatcgagcaccggctgatgatggaggtcagaggtcgcgacccagcagacacacgtgaccacagaaagcagtacccgcccgagctgatgcgcagatttgaggtgtatttccgtcctccggcgacgctgaagccgcgggttgtgcgtgatgtgaaggcggacagcatcgggcagctggtgacggtccgggtaatcgtgacgcgagcgactgaagtcaagcccatgatggccgtcgctacgtacacctgcgaccagtgcggcgccgagacctaccagccgttttaataataaagctcctcctcctgctcctcctcctgctctgctgttctgactctgctcctgtgtgagggtccgctgttgcctgtatatttgcgcttgtttgttgttgtttattttctgcaataaagatgatggccgtcaaaaaaaaaaaaaaaaaaaaaaaa

SEQ ID NO: 104 - protein, 195 aa, Danio rerio
MAPKDYTAEKEKCKRFLQEFYTEDDSGKKIFKYGAQLVSLAHREQVALLVDLDDVAEEDPDLVESVCENAKRYTALFADAIHELLPEYREREAVVKDALDVYIEHRLMMEVRGRDPADTRDHRKQYPPELMRRFEVYFRPPATLKPRVVRDVKADSIGQLVTVRVIVTRATEVKPMMAVATYTCDQCGAETYQPF

SEQ ID NO: 105 - DNA, 1915 bp, Danio rerio
gtatgaatttcttgctttcaagtctgtttagtttcattttattaaggatttaagtgctgcttgattttttaatctactgttacacagaatacagtttttaaaacaggcgttttactttaaaaagaaggacatggcaacagattcgtgggcccaggctgtcgatgaacaagaagctgcagcggaatcgataagcaccttgcaaataagtgaaaaagaggagaagccgacagctgaagcaaatggagcgaagacagaagcgaagaccgaagcaaaaacagaagcgaatgctgataaaacggaggaggatgataaagatgacaaggcagcacagtcactgctgaataaactgatacgcagtaatctagtcaacaccaccaatcaagttgaagtcctccagagggatccgagctctccgctatactctgtcaaatcatttgaagagctgcgactgaaacctcagctattgcaaggagtgtatgccatgggttttaatagaccctccaaaatccaggagactgcattgcccatgatgctcgctgaacctccacagaatctgatcgctcagtctcagtcaagaacgggtaaaacggctgccttcgtgctggccatgttaagtcacgtggacactgagaacaaatggcctgagtgtctgtgtgtgtgccccacatatgagctggccctacagactggcaaggtcatcgagcagatggggaaacattaccctgaagtccagctagtgtacgccatcagaggaaacaaattggagcggggtgcaaaactgcaggagcagatcgtgattggcactcctggtactgttctggattggtgccaaaagctgaaattcatagatcctaagaagatcaaggtgtttgtgttggacgaggctgacgtcatgatcgccacacagggtcatcaagaccagagcgttcgcatccagaggatgcttcctaaaacttgccagatgctgttgttctctgccacatttgaggaaacggtgtggaatttcgcaaagagaatcgttcccgaccccaacattatcaaactcaagcgtgaagaagagactctagacaccatcaagcagtattatgtcatctgcaacagcaaggaggagaaattccaggctttgtgcaacatctacggagccataaccatcgcacaggctatgatcttctgccataccaggaaaactgccgggtggttggcaggagagctgtccagagaaggacaccaggtggcgctgctaagtggagagatgcaggtggagcagagagcagcggtcattgaacgcttccgagacggcaaagagaaagtcctggttaccactaacgtctgtgccagaggtatcgatgtggagcaagtctcagtggtgatcaacttcgatctgccagtggacaaggacggcaaccctgataatgagacgtatctgcaccgaattgggcgtaccgggcgattcggcaaaagagggctggcgatcaacatggcggacagcaagtttagcatgaacaccctcaaccgcatccaggaccacttcaataagaaaattgagaaactggacaccgatgatttggatgagatcgagaagatcgccaactgagcgtcccgcctgtgacgtttacaataggactgtgcccacgctacattttaattcaaaggctggaagcagagctgtgccagagcggatgtttacatgtggagcagttttatctcagatggtgtttttataggtcttttattttgtttcactctttgtacttaataaatgctgaatgtgtagtcttaacgtcacgtctacgagaatcctgcgggagccgtctgggacaccgacaaggagaatatgccttttcaagattaaccgttacatcatgcacctttttaaaatcgcctgccttcaaaaaaaaaaaaaaaaaaaaaaaaa

SEQ ID NO: 106 - protein, 487 aa, Danio rerio
MATDSWAQAVDEQEAAAESISTLQISEKEEKPTAEANGAKTEAKTEAKTEANADKTEEDDKDDKAAQSLLNKLIRSNLVNTTNQVEVLQRDPSSPLYSVKSFEELRLKPQLLQGVYAMGFNRPSKIQETALPMMLAEPPQNLIAQSQSRTGKTAAFVLAMLSHVDTENKWPECLCVCPTYELALQTGKVIEQMGKHYPEVQLVYAIRGNKLERGAKLQEQIVIGTPGTVLDWCQKLKFIDPKKIKVFVLDEADVMIATQGHQDQSVRIQRMLPKTCQMLLFSATFEETVWNFAKRIVPDPNIIKLKREEETLDTIKQYYVICNSKEEKFQALCNIYGAITIAQAMIFCHTRKTAGWLAGELSREGHQVALLSGEMQVEQRAAVIERFRDGKEKVLVTTNVCARGIDVEQVSVVINFDLPVDKDGNPDNETYLHRIGRTGRFGKRGLAINMADSKFSMNTLNRIQDHFNKKIEKLDTDDLDEIEKIAN

SEQ ID NO: 107 - DNA, 1316 bp, Danio rerio
ccacgcgtccgcctgaacgaggcacaacgtggttttaggagaagccatacgacagtaggttcagaacgtttagctttatttatttgaccactgttagatttcaatatgacagaaggcaaatcatcagacaagccggcgaaaagactgctggctctgaatccgaaagaggacgcagaatttcagaagaaagtgcagcaagtgaagaagcgccctcaaacgggtcaaacacttagtccaggagtgctgtatgttggtcatcttcctcgaggactgtttgagcctcagttgaaatcttactttgagcagtttggcaaagtcttacgattgagagtttccaggagtaaaaagactggtgggagcaaaggctatggatttgtggagtttgagtgtgatgaagtggctaagattgtggctgaaaccatgaacaactaccttatgggagaacgcattataaaatgtcacgttattccacctgagaaggttcatgaaaagctctttgtcggctctattgctggcttcaagaagccaaagtatcctgcagtgactcggtataacaaaacacacacagaagatgatgtgaagaaagttggcacaaagcttttaagcaaagagtctaagctgcgcaagagactggcagcaaaaggcattgactatgatttcccaggatttgcagcacagattccagctaaaaaggctccgtcagaagcaaatgtttcagtgtgcagtgaggatgtaactccagtgtgcacaccttcacttttggagaggagaaagtctctcagagttgaggatgatgatgttgatgatgaaatagtcatcaaagtcaaaccagtcccagaaaacagtgatgatgtggaagaaagtgaggaagagtctgctgaagaggatgaaggtgaagaggaggaagcggcttaaaaggagaatttagtgactaataaagactttattcgctgattcagaaggggacgctggactcgcttttcagagccatttgtatgcgattagttctcagttctcccctaaggctggagaagaggaccacacatattactcgcacacattatcctgattgtcaggaatctgttgatttgaggcgtgttatgcagagtttgtgcccgtgaccttttaagggtacaatgtggttgaccatagagttgctgaaatgactgtattaatcaaagcagatgtatagtgttaatatgaaaagaattattttcattgtatttgacacattttctgtccacatatgttgcattgcaatgtttgtaaaacatgtttaaaccgtaaaataaactgctttaataaatgtaaaaaaa

SEQ ID NO: 108 - protein, 269 aa, Danio rerio
MTEGKSSDKPAKRLLALNPKEDAEFQKKVQQVKKRPQTGQTLSPGVLYVGHLPRGLFEPQLKSYFEQFGKVLRLRVSRSKKTGGSKGYGFVEFECDEVAKIVAETMNNYLMGERIIKCHVIPPEKVHEKLFVGSIAGFKKPKYPAVTRYNKTHTEDDVKKVGTKLLSKESKLRKRLAAKGIDYDFPGFAAQIPAKKAPSEANVSVCSEDVTPVCTPSLLERRKSLRVEDDDVDDEIVIKVKPVPENSDDVEESEEESAEEDEGEEEEAA

SEQ ID NO: 109 - DNA, 1940 bp, Danio rerio
aattcggcacgaggtaaaaagcaacaaggcgccgtttgactccgcagaatcggcttttgtacgcggtccgagacctgttcacgggttctgcacacaggcatggcgcgttttctgtagcgtgatttatttttgaatgtcgattttgtaaaacgccaccggcacctgtttatattgtgtttttgtccggatttcaacagctcacccctgtggctttctctgggcgttttgagctgttttgctgctggaaggatgtcaacacaaggaagcgcggttcatgactccgatattcacaatcaggaaaacatgctttcgaggctacgcggtgctgctgcaaagaacagggttgaaaatcgtgaaaatgtcaatcctaaccctaaagcagctaacaacaggacagtcctgggcgagctggagaacagccagaggagacagctcggtctgcgaggcgctaaacaggggtctgggccacagataattgcgtgtaaacctgaagaaaatgccaggagttttggagagaggcctagcaacagacagcctgctgcttttcagattcacgtggatgagcctgatggcgcgtgctccaagaaagcacctttacagagatccaccatggactgttccccgctgacactaaaccccactgtcacccgcctcaggcagcccctcgccaccattgaccttccactggaggccagttttgattctccaatggacatgtcagtaatcgattgtgaggaacgaccaacaaatgtcaatgaagtctcagattatgcagcagaaatccacacgcatttgcgggaaatggaggtcaagtctaaaccgaaagcaggttacatgagaaaacagccagacatcacaaacagcatgcgtgctattctagtggactggttggtggaagtgggagaagaatacaagctacagaacgagactctttacctggctgtaaactacattgatcgctttctgtcctccatgtctgtgctgagggggaaactgcagctggtgggcacggctgctatgcttttggcttcgaagtttgaggagatttaccctccagaggtggcggagtttgtgtacatcactgacgacacgtacacaaagaaacaagtgttgcggatggagcatctggtgctgacagttctctcgtttgatctggccgctcccaccatcaatcagttcctcacccagtatttcttacaccagcctgtgagcagcaaagtcgagagcttatcaatgtttcttggagagctcagcttgatagattgcgatcccttcctcaagtatctcccgtctcaaatggctgctgctgctttcatcttggccaaccacacactggccagtggatcatggtcgaagtctcttgttgatttgacgggttactctttggaggatcttctgccgtgtgttcaggatctccatcagacatatctcgctgcttctcaacacgctcaacaggctgtcagagagaaatacaagggctcaaagtaccatgaggtgtccctgatcgagcctccagagaagctgatgctgaactaaccctccatcaacattgtgttcatgtcttccagaactctgttaggaatcgtgcgcacactttgatgctttattaccataactccaataactgaagccatagcctcaatttggggaaatggtgggattttagtgatctggacatttaactttttttttatttttatttttttatttgcagtccttaaggattgtgccctttttatgcttgattcagccagatatttgtacatttttaggtaatcaagctttgtttggatatttcagtgtttgtactctatttttatgaaagactccagcagtggactttctattttccactgttaaagtgaccttccctttacatccaaataaaagtgctgctggcctttaaagcacaaaaaaaaaaaaaaaaaaaaaaaaaaaaa

SEQ ID NO: 110 - protein, 428 aa, Danio rerio
MSTQGSAVHDSDIHNQENMLSRLRGAAAKNRVENRENVNPNPKAANNRTVLGELENSQRRQLGLRGAKQGSGPQIIACKPEENARSFGERPSNRQPAAFQIHVDEPDGACSKKAPLQRSTMDCSPLTLNPTVTRLRQPLATIDLPLEASFDSPMDMSVIDCEERPTNVNEVSDYAAEIHTHLREMEVKSKPKAGYMRKQPDITNSMRAILVDWLVEVGEEYKLQNETLYLAVNYIDRFLSSMSVLRGKLQLVGTAAMLLASKFEEIYPPEVAEFVYITDDTYTKKQVLRMEHLVLTVLSFDLAAPTINQFLTQYFLHQPVSSKVESLSMFLGELSLIDCDPFLKYLPSQMAAAAFILANHTLASGSWSKSLVDLTGYSLEDLLPCVQDLHQTYLAASQHAQQAVREKYKGSKYHEVSLIEPPEKLMLN

SEQ ID NO: 111 - DNA, 1125 bp, Danio rerio
tgcgcttgctcaatttgggcaatattttgaaactttccaatcaaaccaatatactttttcggcttatctgacatatttttaacggaaaaactcggatccatcgcgaaataacggaattatccagcctatcatgtcacaaacagaaagcagcatggagcagcagcgggaagaagagccgagccaactcgacccggccggtccggcagaggatgatacttcagaaaatggccatgatgaagcatcagatacagctatggatagtgaagcacagtcgtcatcacctccctcagcaaagggaaaggacctgctgggtggatatgaggaaaaagtgcagacagaccggaccaacagatttgagtacctcttgaagcagacagaagtgtttgctcatttcattcaaccagcagcccagaagaccccaacttcacctttgaagatgaagcctggccgtcctcgtattaaaaaagatgagaagcagaacctgctgtctgctggagataaccgccatcgtcggacggagcaggaggaggatgaggagctgctaagtgagaacagtaaagccaccagtgtctgcacacgttcaagtctcgcacagccatggagcttcagaggagatgcaacacactgatcacactcatcgaacgtgaaaacatggagttggaagagcgagagaaagccgagaagaagaagagaggaccgcgcacatcttcggctcagaaacgcaagcaggacgggacccctgatggacgtggccgcaagaaaaagctgaaattgtgagcaggcaggcatttcacacacctcactcggcgaggagcttcagtacagcaatactgcattgattgttacgggtcccactcatgtactgtatggatttgcagcactgatcctcgcctctcaagtagcttggccttcttaacaaggtgtagagttgtaaattaggtctcttttagttatataatgtaactacggctgtgctgtcggatgtgttttgtatttatggctacttcaaatttttttttgtaccacattccatttgcttgtatcagtttaatttgcagtctttaccccctcattattagtgtcttcagtattgtattgtctctgtatccgccattggaaagtgac

SEQ ID NO: 112 - protein, 213 aa, Danio rerio
MSQTESSMEQQREEEPSQLDPAGPAEDDTSENGHDEASDTAMDSEAQSSSPPSAKGKDLLGGYEEKVQTDRTNRFEYLLKQTEVFAHFIQPAAQKTPTSPLKMKPGRPRIKKDEKQNLLSAGDNRHRRTEQEEDEELLSENSKATSVCTRSSLAQPWSFRGDATHTLIERENMELEEREKAEKKKRGPRTSSAQKRKQDGTPDGRGRKKKLKL

SEQ ID NO: 113 - DNA, 4221 bp, Danio rerio
gggttcgtgctctgagtttacgcgccatttatgagtgggatttcagtgcagggaagtaaacgagcgcagatctaccctcgttttacaccacaaacactcaatatacgcacttttattcaaaaggagagcaaaaagcggagtaaaatcgacgagaaagagggaaatcagcggaatcgaaggtgccgtgaataatgcctgtaaaaacaagcaagacggccggtcggagagcggcggaggactcggatgaggaggtgagcaaaccgcgcaaagcaccgagcactgccgtccagagcagcccgccgcccgcccttaacgatggggctgcgggagagtctgctgaggtggttgatagccgcagtctggaggagattctgggtagcatccctcctgcaccgcccccagccatgagctcagagcccggtgccccgcgactcatgatcacgcacatagtcaacagaaacttcaagtcctacgctggagagcagattctggggcccttccataagcgcttttcctgcatcattggtccaaacggcagcggcaagtccaacgtgatcgactccatgctgtttgtgtttggatatagagctcagaaaatccgctcgaagaaactgtcggtgctgatccacagttctgatggtcatccagacatccagagctgcactgttgaggtccactttcagaagatcattgataaggaaggagacgactatgatgtcatccccgacagctccttctacgttgccagaacggctggaaaagataattcctctgcctactacattaataccaagaaggcaaccttcaaagatgtgggcacattactgcgcagccacggcatcgatctggaccacaatcgtttcctcatcctgcagggtgaggtggagcagatcgccatgatgaagcccaaaggtcagacggagcatgatgagggcatgctggagtacctggaggacatcatcggctcctgtcgtctcaaagagcccatcaacatcctctgtcggcgggtggaggcgctgaacgagcagcgaggagagaagctgaatcgagtgaagatggtggagaaagagaagagcgctctggagggagagaagaataaagctgtggattttctcaacttagaaaatgacatctttaagaagaggaacctcctctgtcacttctatgttcatgatctgcagagccgtgtgtccgtgaaagaagctgagaaacagcagattcaggaggacacaaaagatttgagtgacaaaagcagccagctgactgaagaaatgaagagcaaaaacgaggacctgaaggctgtggagaaaaaattgaccaagctgaccaagtacatcgagagtcaaaaggagaagttcacccagcttgacctgcaggatgtggaggtgcgcgagaagctcaaacacaccaagagcaaaaccaagaaactgcagaaacagcttcagaaagaccaggagaagttggaggaggtgcggggtgttcctgcgagcagcgagaagatcatcactgaggcatccgctcagaaggaggagctggagaaaaagaagctactagaggagcagaagttagcagaggtgatggagagcctgaaggaggagaccaaaggcctacaggacgataaagagaaaaaagagaaggagctgctggagttaagtaagtcggtgaacgagactcgctcacggatggatgtggcgcaatcagagctggacatctacctgagtcagcacaacaccgctataaaccagctcaaccaggccaaaagtgctctgcaggagaccgtagacacgctgagagagaggagagccgccattaaagacctccaggtcaaaataccagctcaagaggagcagctcaagaaggacgagcgggagctggagcagatctccgagcaggacaaacagacgcgagcgcaggtcggggacatgagacagaaggtggctgaagccaagagctctctgtcctccaaccgcagccgcagcaaagtgctggacaccctcatgcagcagaaacgctcagggaagatacctggcatcttgggacgactgggtgatctgggagccattgatgagaagtatgacgtcgccatctcctccagctgcggctcgctggacaatattctagtggacaccattgacaccgcgcagaagtgcgtcacatttctcaaggcccagaacatcggtgtggccacattcatcggcttagacaagatgaaagtgtggcagcagagcatgggctccatctctaccccggagaacatccctcgtctgtttgatatggtgcgggtgaaggatgagagcgttcgtcctgctttttacttcgccctgagggacactctggtggctaaagacctggagcaggccacgcgggtcgccttccagaaggacaagcgctggagggttgtcactctgcaggggcagatcatcgaacaggccggaacgatgactggaggaggcgggcgggtgatgaagggcatgatgggctcctcattctgtgctgatgtcacacaggagcagctggataaaatggagagtgctttgaacaaggaggtgacgcagctgcaggactgccagaagaggaaaaaccagctggaggagaaggtgcacaaagccagatgtgagctgagagacatgaagaacacgctggagaagtacacagccaccattcagagcctcacagagcaggagcttcacctcaagcctcagatcaaggacctggaggcaaacgtgatcgccgctgctcctgataaagccaagcagaagcagatggagaagagtctggaggcctacaggaaagactttgaggcagcgtccaataaagcaggaaaggtggaggccgaggtgaagcggctgcacacgctcatcgtggacatcaacagtcacaagctgaaggcgcagcaggacaaactggaccagatcaacacacagctggaccagtgctcctcagccgtcaccaaagcccaggtggccatcaagactgcgggacgtaatctgaagaaatcagaggacggtgtgagtcggctggagcaggagatcagtgagaatgagaagctgatggaggagctgacggagcagctgaagaaactggaggagcaggctggagaaatcatgcagtccaccagcagcggagaaggctttgcccgaggtgcaggatcagcatcgtgcagtggtgcaggagatcaaggctctacaggagcaggagcacgcgttgcagaaagagtttctgagcgtccggttgaaggtggagcacatcgacacggccatcacggagtgccacaacaagatcaaacactggcagaaagaggccagcaagttgtgtcttcaccacattgacggagtgcctgcagaagagctgcccgccctgaacccagatcagctgcaagacatcggcaatccagacgtcatcaagaatgagatcgctctgctggaggaccgctgcgccaacatgaagcccaacctgggggctatcgcagagttcaagaagaaggaggagttgtatctgcagagagtggcagagcttgatgacatcactacacagagagacagcttcaagaggggctgcgaggacctgcgcaaacagagactgcacgagttcatggcgggattcaacatcatcaccaataaactgaaggagaactatcagatgctcacgctagggggcgacgcagagctggagctggtggacagtctggatcctttctctgagggcatcatgttcagtgtacgccccccaaagaagagctggaagaagatctataatctgtctggaggagagaagacgctcagctctctggcgctggtgtttgctctgcatcactttaagcccacaccgctctacttcatggacgagatcgacgctgctctcgacttcaagaacgtctccatcgtggcctgctacatatatgaacaaaccaagaatgcccagttcatcatcatctctctgaggaacaacatgtttgagatggccgaccggctcattggcatctacaaaacgcacaaccccactaagaacgtggccatcaaccccaaaaccatcatcctgcgagagatccagacggcatgagcgcagactcagtaatgcaccatcacctgttctgctttcacatcgctgtgctcctgtgagccttttacatgtctcccgttactctgctttttaattttaatttttaataaaatgatttttattcgccagaaaaaaaaaaaaaaaataaaaaaaaaaaaaaaa

SEQ ID NO: 114 - protein, 1287 aa, Danio rerio
MPVKTSKTAGRRAAEDSDEEVSKPRKAPSTAVQSSPPPALNDGAAGESAEVVDSRSLEEILGSIPPAPPPAMSSEPGAPRLMITHIVNRNFKSYAGEQILGPFHKRFSCIIGPNGSGKSNVIDSMLFVFGYRAQKIRSKKLSVLIHSSDGHPDIQSCTVEVHFQKIIDKEGDDYDVIPDSSFYVARTAGKDNSSAYYINTKKATFKDVGTLLRSHGIDLDHNRFLILQGEVEQIAMMKPKGQTEHDEGMLEYLEDIIGSCRLKEPINILCRRVEALNEQRGEKLNRVKMVEKEKSALEGEKNKAVDFLNLENDIFKKRNLLCHFYVHDLQSRVSVKEAEKQQIQEDTKDLSDKSSQLTEEMKSKNEDLKAVEKKLTKLTKYIESQKEKFTQLDLQDVEVREKLKHTKSKTKKLQKQLQKDQEKLEEVRGVPASSEKIITEASAQKEELEKKKLLEEQKLAEVMESLKEETKGLQDDKEKKEKELLELSKSVNETRSRMDVAQSELDIYLSQHNTAINQLNQAKSALQETVDTLRERRAAIKDLQVKIPAQEEQLKKDERELEQISEQDKQTRAQVGDMRQKVAEAKSSLSSNRSRSKVLDTLMQQKRSGKIPGILGRLGDLGAIDEKYDVAISSSCGSLDNILVDTIDTAQKCVTFLKAQNIGVATFIGLDKMKVWQQSMGSISTPENIPRLFDMVRVKDESVRPAFYFALRDTLVAKDLEQATRVAFQKDKRWRVVTLQGQIIEQAGTMTGGGGRVMKGMMGSSFCADVTQEQLDKMESALNKEVTQLQDCQKRKNQLEEKVHKARCELRDMKNTLEKYTATIQSLTEQELHLKPQIKDLEANVIAAAPDKAKQKQMEKSLEAYRKDFEAASNKAGKVEAEVKRLHTLIVDINSHKLKAQQDKLDQINTQLDQCSSAVTKAQVAIKTAGRNLKKSEDGVSRLEQEISENEKLMEELTEQLKKLEEQAGEIMQSTSSGEGFARGAGSASCSGAGDQGSTGAGARVAERVSERPVEGGAHRHGHHGVPQQDQTLAERGQQVVSSPHGVPAEELPALNPDQLQDIGNPDVIKNEIALLEDRCANMKPNLGAIAEFKKKEELYLQRVAELDDITTQRDSFKRGCEDLRKQRLHEFMAGFNIITNKLKENYQMLTLGGDAELELVDSLDPFSEGIMFSVRPPKKSWKKIYNLSGGEKTLSSLALVFALHHFKPTPLYFMDEIDAALDFKNVSIVACYIYEQTKNAQFIIISLRNNMFEMADRLIGIYKTHNPTKNVAINPKTIILREIQTA

SEQ ID NO: 115 - DNA, 2931 bp, Danio rerio
gtctacagtcgagcttctgttcaaggcagacgtgagaaaatggcggattcctcagagtcgttcaacatggccaccagccccactcgaggctctcggcggggggatctgacctccagccctggcagagaccttcctccatttgaggatgagtctgagggacttctgggagacactcttcctgatgaggaggacgatgatggagaggagctgattggagacgccatggagagagactaccgtgtagtcccagagctggaccgctatgaagcggagggtctggatgaggatgaagatctgagtgaactttcacccagcgctcgtgccgaggcagaagcggcgatgaggagacgcgacagagaacaaggccttggcatgggccgcatcggacgtggactcctttacgacagtgaagacgaggatgacaaacggcctacgaagaggcagcgtgtcctggcagagagagcagccgaaggtggagcgatggagggcgaggatgaagagatgatcgaaagcattgagaacctgggacatgaagggccacacggtgcgcgagtgggtttctatggccgcaccacgactggagatctaccaccgcttaagaacttcctgcgcacgcacgtggatgagcacggacataatgtgtttaaagagcgcatcagcgacatgtgcaaagaaaataaggagagtttgctggtgaactatgaagactggcttccaagagcatgtgttggcgtattttctaccaaagctcctgccgaaatgctgaagatctttgacgaagcagcaaaagaagttgtacttgccatgtaccccaaatacgaccgaattgcacatgaaatccacgtcagaatcggtaatctaccattgggatcactcaggcaactccagctgatccccactagtggtgtcgtgaccaactgcactggagttcttcctcaacttgggatggtcaaatacaactgtaacaagtgtaactttattttggggccggtctttcagtctcaaaaccaggaggtgaagccgggctcctgtcctgaatgtcagtcgctcgggcccttcgaaatcaacatggagcagacggtgtaccagaactaccagcgcatcaccattcaggagagtcctgggaaagtggctgctggtcgcctgcctcgatctaaagacgccatcctgcttgcagacctggtggacatgtgtaaacccggagatgagatcgagctcactggaatctaccacaacaactatgacggctctctaaacatggccaatggtttccccgtcttcgccaccgtgatcttagccaatcacatcgctcgaaaggacgagggcgtggctgtggcggagctcaccgatgaagacgtcaaagccatcgttgcactgtccaaagacgagcgcattggcgaaaggattttcgcaagtattggacccttcatctacggacacgaggacattaaacgtggtctggcacttgctctgtttggtggcgaagctaaaaatccaggtggaaagcacaaggtgcgtggtgatataaacgtcctcctgtgtggagatcccggcacggctaaatctcagttcctgaagtacgtggagaaggtggcgagtcgagcggtgttcaccacaggacagggagcctccgctgtgggtctgacggcttacgtgcagcggcatcccgtcagccgagagtggacgctggaggccggagcgttagtgctggcggacagaggagtgtgtcttattgatgagttcgataagatgaacgatcaggacagaaccagcattcacgaggcgatggagcagcagagcatctccatttccaaagcaggaatcgtgacttctctccaggcccgctgcaccgtcatcgctgccgccaatcccatcggtggacgctacgacccgtctctaacgttttcggaaaacgtggacctgacggagcccatcatttctcgatttgatgttctgtgtgttgtaagagacactgtcgatcctgtgcaggatgaaatgttggctcgctttgtggtcggcagccacatcaagcatcaccctagcaacaaagaagggggcgtggccggactggaggaggtggtgcttcccaacacattcgacgtcccaccaatcccgcaggagttgctccgcaagtacatcatttacgctaaagagcgcgtgaggcccaaactcaaccagatggaccaggacaaagtggcccgcatttacagcgacctacgaaaagagtctatggctacaggaagtatcccgattactgtgcgtcacatcgagtctatgatccgtatggccgaggctcacgctcgcatgcatctgcgcgactacgtgctggaggacgacgtcaacatggccatccgcgtcatgctagagagcttcatcgacacgcagaagttcagtgtgatgaggagcatgagaaagacgttcgcacggtatctggccttcagacgagacaacaacgagctgctactgttcatccttaagcagctggtttctgagcaggtctcatatcagcgcaaccgttacggagcccagcaggacaccatcgagatcgcagagaaagacctggtggacaaggctcgacagatcaacatccacagcctgtcagcgttttacgacagcgatctcttccgctcaaacaaattctcccacgatgtcaagaagaagctcatcgttcagcagttctagcctgcatctggaatctcccggacttgtatataatttcgtcacgtggtcgttggaccagtcgttcgttacacatggattccacattttgacccattcatgaacttgctaccatatagatctgcctggctctcgtggttttgttgactgtatcttgcattgtttctgctgtcaaattaattgaatgttgtaaaaaaaaatatgagagaaatttttgtactgtttgttcagagtaataaactgaagctgtgt

SEQ ID NO: 116 - protein, 880 aa, Danio rerio
MADSSESFNMATSPTRGSRRGDLTSSPGRDLPPFEDESEGLLGDTLPDEEDDDGEELIGDAMERDYRVVPELDRYEAEGLDEDEDLSELSPSARAEAEAAMRRRDREQGLGMGRIGRGLLYDSEDEDDKRPTKRQRVLAERAAEGGAMEGEDEEMIESIENLGHEGPHGARVGFYGRTTTGDLPPLKNFLRTHVDEHGHNVFKERISDMCKENKESLLVNYEDWLPRACVGVFSTKAPAEMLKIFDEAAKEVVLAMYPKYDRIAHEIHVRIGNLPLGSLRQLQLIPTSGVVTNCTGVLPQLGMVKYNCNKCNFILGPVFQSQNQEVKPGSCPECQSLGPFEINMEQTVYQNYQRITIQESPGKVAAGRLPRSKDAILLADLVDMCKPGDEIELTGIYHNNYDGSLNMANGFPVFATVILANHIARKDEGVAVAELTDEDVKAIVALSKDERIGERIFASIGPFIYGHEDIKRGLALALFGGEAKNPGGKHKVRGDINVLLCGDPGTAKSQFLKYVEKVASRAVFTTGQGASAVGLTAYVQRHPVSREWTLEAGALVLADRGVCLIDEFDKMNDQDRTSIHEAMEQQSISISKAGIVTSLQARCTVIAAANPIGGRYDPSLTFSENVDLTEPIISRFDVLCVVRDTVDPVQDEMLARFVVGSHIKHHPSNKEGGVAGLEEVVLPNTFDVPPIPQELLRKYIIYAKERVRPKLNQMDQDKVARIYSDLRKESMATGSIPITVRHIESMIRMAEAHARMHLRDYVLEDDVNMAIRVMLESFIDTQKFSVMRSMRKTFARYLAFRRDNNELLLFILKQLVSEQVSYQRNRYGAQQDTIEIAEKDLVDKARQINIHSLSAFYDSDLFRSNKFSHDVKKKLIVQQF

SEQ ID NO: 117 - DNA, 623 bp, Danio rerio
aaacctcaacgacctgcgacgacgaaatgaaaagcgagcagcaaagctgttgaataacgcttttgaagagctcctggcattccagcgagccctgaaagatctggtggcttcaatagatgccacttacgccaagcaatgtgaggagttcttcattggtttggagggcagctttgggagcaaacatgtctcccctcgaaccctgacctctcgcctgctgggcagcatggtgtgtttggagggcatcgtcactaaatgctctttggtgcgtcccaaagtagtacgcagtgtgcactactgtccagccactaaaaagaccatggagcgcaaatacaccgatctgacttctctggatgccttcccttcaagtgctatttaccccaccaaggatgaagagaacaatccattggagacagagtttggtctgtccgtctataaggaccaccaaaccatcactatacaggagatgccggagaaggcccctgctggtcagctgccccgctctgtggacatcatcctggacaatgacctggtggacgcccgtgaaacctggagaccgaacacaggtgatcggcacctaccgctgcctgccgggcaaaaaaggtggcttacctcgggcaccttca

SEQ ID NO: 118 - protein, 178 aa, Danio rerio
NLNDLRRRNEKRAAKLLNNAFEELLAFQRALKDLVASIDATYAKQCEEFFIGLEGSFGSKHVSPRTLTSRLLGSMVCLEGIVTKCSLVRPKVVRSVHYCPATKKTMERKYTDLTSLDAFPSSAIYPTKDEENNPLETEFGLSVYKDHQTITIQEMPEKAPAGQLPRSVDIILDNDLVD

SEQ ID NO: 119 - DNA, 2915 bp, Danio rerio
aggctgacgtttccgtactgctgcctacagatggacactctctacatctcttcccaccctgatgacttcaggagtttgttgcccttgatcgctgctggattctgtccgtctcctccacgaattctccaccaggaccctccagcagaggtggccagatctcggcctgcgctggttttagcaggtgcggggggcactgtgctcagcggcaccagtgctgtgagttggtatctggcagcaacaggcaaacgagccggaagtgataaaaagcaggagagtcaggtgtggcagtggctgagttttgctgagaatgagctgacgccggtggcctgcgctgtggccttccctctgcttggaatcatgggagttgataagaagcttcagcagagttccagagcggagctgctgcgtgttctgaaagctctggatgggactcttgcgcttcggacattcttggtgggagagagtgttactctggcggatgcacctgttgcgatggcggctttgctgccattcaaatatacactggaccctgcaaacaggaagtcgttggtgaatgtgaccaggtggtttaatacctgtgtaaatcagccacagtttctgaaggtattgggtaaaatctcactatgtgagaaaatggtgcctgtgacccccaaacccaacactgcttccaatgtcacagtaactgctgctgctgctgctaaaccagacagcggacctgctaatggtcctcctaagacagaagctcaactgaagaaagaggccaagaagcgagaaaagatggagaagtttcaacagaagaaagacatggaggagaagaagaaaatgcagcctcagactgagaaaaaggccaaaccagagaagaaggaactgggtgtgatcacctacaatgtctccacaccacctggcgagaaaaaagatgtcctgagtcctctgcctgattcatacagtcctcagtatgtggaagcggcctggtactcctggtgggagaaacaaggatttttcaagcctgaatctgagaggaagaaactgagtgagccgaatccgcgtggagtgtttatgatgtgtatcccaccacccaatgtgactggatcccttcaccttggtcatgccttgaccaatgctattcaggactgcttgacgagatggcacagaatgcgagaagagacaacactgtgggatcccggctgtgatcacgctggcatcgccacacaggtggtggtggagaagaaactcgtgagagagcggaagatgaccagacacgaccttgggcgagagaacttcatcaaggaggtctggaagtggaagaagccagagaaaaagctgccaaacgcttcaatgtcactgcagacaaaatctctctgcgccaggatgaggacgtgttggacacatggttctcctcggggatcttccccttctccatctttggctggccaaatgagtctgaggatctgagagtcttctacccgggcacactgttagagacgggtcatgatatcttgttcttctgggttgctcgaatggtcatgatgggcctcaaactcactggaaaactacctttcaaagaggtgtacctgcatgcagtggtgcgagatgctcatggcaggaaaatgagcaagtctcttggtaacgtcatcgaccctctggacgtcatcaccggcatatcactggagggtctttatgctcagctagcagacagtaatctggatcctctggagattgagaaagccaaacagggacagaaatccgacttatcccactggaattccagaatgtgggactgatgcgcttagatttgctctgtgtgcataccccagtcaaggtcgagacattaatctggatgttaacaggattttgggatacaggcatttctgtaacaagctgtggaacgctgtgaagttcgccatgaggactctgggagaggggtttgtgccttgtgagaaagctcagctgtgtggaagtgaaagtgtctctgatcgctggattctgtctcgcctgagcgcagcagtagcgttgtgtgatggcggatttaaggcctacgactttcccacaatcaccactgccatctacaatttctggctgtatgagctctgtgacgtctatttggagtgtgtgaagccggtgtttagcaggacagactctgagggtcagaaacaggctgacgtctgcagacagaccctctacacctgtctggaggtgggactgcgcctcctgtcccccatcatgccttttgtttctgaagagctcttccagcgtttgccaaggagaaggcctcgggacagccttccagcatctctgtcacacctatcctgacactgctgagttctgctggcacagtgaggatgtggaccgtcagatggagtttgtcatgtctgtggtcagaaccattcgctcactcagggctgattacaacctgactaagactcgcgctgactgtttcctgcagtgtatcgactccgaaacggccgctctggttcagaaatacagtcttcagatccagaccctgtcctactctcaggccgttcactctgtggtcggagatgccgctatcccacaaggctgtgcggtggccatcgcttcagataaatgcactgtcaacctcatgctgaagggtttgattgacttgggaaaggaggtcacgaaactgacggccaagaaaggcgagctggagaaacagatggagaagatgaaggaaaagatgacaaagaacgactacaaggagaaagttcctgtgaaagtgcaggaggctgatgctgagaagctgaggcagagtgaagcagagctgcagaaggtgaaggaagctatagagaacttcagcaagatgatgtaacatttcaccctcatgctttttttttgtatcgtttatgaatttttccattgaaattcgttcctttgtgttcaatgtcagtcatgcctag

SEQ ID NO: 120 - protein, 957 aa, Danio rerio
MDTLYISSHPDDFRSLLPLIAAGFCPSPPRILHQDPPAEVARSRPALVLAGAGGTVLSGTSAVSWYLAATGKRAGSDKKQESQVWQWLSFAENELTPVACAVAFPLLGIMGVDKKLQQSSRAELLRVLKALDGTLALRTFLVGESVTLADAPVAMAALLPFKYTLDPANRKSLVNVTRWFNTCVNQPQFLKVLGKISLCEKMVPVTPKPNTASNVTVTAAAAAKPDSGPANGPPKTEAQLKKEAKKREKMEKFQQKKDMEEKKKMQPQTEKKAKPEKKELGVITYNVSTPPGEKKDVLSPLPDSYSPQYVEAAWYSWWEKQGFFKPESERKKLSEPNPRGVFMMCIPPPNVTGSLHLGHALTNAIQDCLTRWHRMREETTLWDPGCDHAGIATQVVVEKKLVRERKMTRHDLGRENFIKEVWKWKKPEKKLPNASMSLQTKSLCARMRTCWTHGSPRGSSPSPSLAGQMSLRIVFYPGTLLETGHDILFFWVARMVMMGLKLTGKLPFKEVYLHAVVRDAHGRKMSKSLGNVIDPLDVITGISLEGLYAQLADSNLDPLEIEKAKQGQKSDLSHWNSRMWDZCACAYPSQGRDINLDVNRILGYRHFCNKLWNAVKFAMRTLGEGFVPCEKAQLCGSESVSDRWILSRLSAAVALCDGGFKAYDFPTITTAIYNFWLYELCDVYLECVKPVFSRTDSEGQKQADVCRQTLYTCLEVGLRLLSPIMPFVSEELFQRLPRRRPRDSLPASLSHLSZHCZVLLAQZGCGPSDGVCHVCGQNHSLTQYPDTAEFCWHSEDVDRQMEFVMSVVRTIRSLRADYNLTKTRADCFLQCIDSETAALVQKYSLQIQTLSYSQAVHSVVGDAAIPQGCAVAIASDKCTVNLMLKGLIDLGKEVTKLTAKKGELEKQMEKMKEKMTKNDYKEKVPVKVQEADAEKLRQSEAELQKVKEAIENFSKMM

SEQ ID NO: 121 - DNA, 645 bp, Danio rerio
ctcgagtgcgccattcacacacacgctgcgaagatggctgaagattgggaggctgcaccagctgttgctgaaaccccagagatcaaactctttggcaagtggagcacagatgatgtacagatcaatgacatctccctgcaggattacattgccgtgaaggagaaatacgccaaatacctccctcattccagcggcagatacgcagccaagcgtttccgtaaggctcagtgtcccattgtggagcgcgtcactaactccatgatgatgcacggacgcaacaacggcaagaaactgctgaccgttcgcatcgtcaagcacgcttttgagatcatccacctgctcactggcgagaatcctctgcagattctggtaaacgccatcatcaacagtggccctcgtgaggactccacccgtatcggacgtgctggaaccgtgaggagacaggctgttgatgtgtcccccctgcgcagagtcaaccaggccatctggctgctctgcactggtgcaagagaagctgctttcaggaacatcaagaccatcgcagagtgtcttgctgacgagctcatcaacgctgccaagggttcctacaactcttacgccatcaagaagaaagatgagctggagagagtggccaagtctaaccgc

SEQ ID NO: 122 - protein, 204 aa, Danio rerio
MAEDWEAAPAVAETPEIKLFGKWSTDDVQINDISLQDYIAVKEKYAKYLPHSSGRYAAKRFRKAQCPIVERVTNSMMMHGRNNGKKLLTVRIVKHAFEIIHLLTGENPLQILVNAIINSGPREDSTRIGRAGTVRRQAVDVSPLRRVNQAIWLLCTGAREAAFRNIKTIAECLADELINAAKGSSNSYAIKKKDELERVAKSNR

SEQ ID NO: 123 - DNA, 1106 bp, Danio rerio
cctccctgtgctgttcttcctgtcgcgtgcgggtctcctcagcacatctaatcctcatcatggcgtctctatcgatggctccggtcagcattttcaagcatggagcggatgaggagaaggcagaaaccgctcgcctgtcgtccttcatcggcgccatcgccatcggggatctggtgaagagcactctggggcccaaaggaatggataagatcctcctgggcgggggcagagagagcaccgtgacggtgaccaatgacggagccaccatcctcaaggccatcggggtggacaaccctgctgctaaagtgctcgtggacatgtctaaggtccaggatgatgaggttggtgacaggacgacctcggtcactgtgctggctgctgagctgctgcgggaggcagagctgctgatcgccaagaagattcatcctcagatcatcatcgccggctggaggaaggccacacaggcggcccgcgacgccctcagagaggccgcagtggatcatgggagtgacgaggtgaagttccaggaggatctgctgaacatctcccgcaccacgctctcctccaagctgctgacgcaccataaggaccacttcgccaagctggcggtgcaggcggtgatgcggctccggggatctggcaacctggaggccattcacctgatcaagaagctgggcggcagcctcactgactcatacctggacgaggggttcctgctggacaagcgtatcggggtcaatcagcccaaacgcatcgagaacgctaacatcctcatcgccaacaccggcatggacaccgacaagatcaagatcttcggctccagagtgcgtgtggactccactgctaaagtggcagagatcgagacggcagagaaggagaagatgaaggagaaggtggagcgaatcctcaaacacggcatcaactgcttcatcaacaggcagctgatctacaattacccggagcaattgtttgcggccgcaggcgtcatggctatcgaacacgcagatttcacaggagtggagcgtctggctctcgtcacaggtggggaaatcacctccacctttgaccaccccgactggtgaactgggccactgtaagctgatcga

SEQ ID NO: 124 - protein, 340 aa, Danio rerio
MASLSMAPVSIFKHGADEEKAETARLSSFIGAIAIGDLVKSTLGPKGMDKILLGGGRESTVTVTNDGATILKAIGVDNPAAKVLVDMSKVQDDEVGDRTTSVTVLAAELLREAELLIAKKIHPQIIIAGWRKATQAARDALREAAVDHGSDEVKFQEDLLNISRTTLSSKLLTHHKDHFAKLAVQAVMRLRGSGNLEAIHLIKKLGGSLTDSYLDEGFLLDKRIGVNQPKRIENANILIANTGMDTDKIKIFGSRVRVDSTAKVAEIETAEKEKMKEKVERILKHGINCFINRQLIYNYPEQLFAAAGVMAIEHADFTGVERLALVTGGEITSTFDHPDW

SEQ ID NO: 125 - DNA, 2070 bp, Danio rerio
ggcaccgtcctcctctattccgcaatcatgatgtccactccagtcatcctcttgaaagagggcacagacacctctcagggggtcccacaactggtcagcaacataaatgcctgccaggttgtggcagaggctgtgcggaccacccttggcccccgtggcatggacaagcttgtggtggataaccgaggcaaagccactatttctaatgatggagccacaattctgaagcttttggatgttgtgcatcctgcagccaagactctggtggacattgctagatctcaagatgctgaggtcggagatggtaccacttcagtgactctgcttgctgctgagtttctgaagcagttgaaaccgtatgtggaagaagggcttcacccacagaccatcatcagagcattccgcatcgccacccaacttgctgtcaaaaagatcaaagaaatcgctgttaccatcaaaaaggatgacaaacaagaacagaggaggttgttggagaagtgtgctgctacagctttgaactccaagctgatagcagggcagaaggatttcttctccaagatggtggtggatgcagtgatgatgctggatgatctgctgcctctgaagatgattggagtgaagaaggtgcagggtggtgctctggaggagtctcagcttgtggctggtgtggcatttaagaagactttctcttatgctggttttgagatgcagcccaagcgttacatgaacccaaaaattgccctgctcaacattgagctggagttgaaggcagagaaggacaatgccgaggttcgcgtcaactcaatggaggactatcaggccattgttgatgctgaatggaacatcctgtatgataaactggagaagatccacaaatctggcgctaaagttgtgctgtccaagctgcccattggagatgtagccacacagtactttgcatacagaaatctgttctgtgcagcccgcgtcgtggaggaaaatctcaaaagaactatgatggcttgtggtggctccattcagaccagtgttggttccctgactgatgatgttcttggccagtgtgagctatttgaagaagtgcaggttggaggagagagatacaatttctttaaaggctgcccaaaggccaagacctgcaccatcattctgaggggtggtgcagagcagtttatggaagagacggaccgctcactgcatgatgccattatgatagtgcgcagggcaatcaagaatgactccattgttgccggtggtggggcaattgagatggagttgtcgaagtatctgagggattattctagaacaattccagggaagcagcaattgctgatcggagcctatgccaaggccctggagatcattcccagacagctctgtgacaatgcgggatttgatgccacaaatattctaaacaaactgagggccaagcatgcacagggtggtatgtggtatggagtggatgtgaataatgaagacatagcagataacttccaggcatgtgtttgggagccctctatagtgcgtatcaatgccttgactgctgcatctgaagctgcatgcctcatactgtcagtggatgagaccatcaagaaccctcgctccagtgttgatggcccaccggcagctgctggcagaggaaggggtcgtggtagaccagcccatgcccactaatccatatccagccttaggactttcagttgatcacaacaaatctttgccgttcacttggttttgatttgttttattggcccgttttgttatttaccagtcccactaactacgagaatctctgtgtttgcaaaggctaatgcagaataaaatggcgtttttttttctctctccttgttttagaccaatgggaactgacatcttttctattgggtatttggaattttgaatgttgttttgtaatagtctgagaacacacaaaaaacaccatgtgctcaaaattggttggtttgtagatctgagtaagcactgaaatgcgggcactctgctgagatatgttcctgtgttggtcagttggtggaactatttattgtaccattaaaatcagtacttttgcctcaa

SEQ ID NO: 126 - protein, 547 aa, Danio rerio
MMSTPVILLKEGTDTSQGVPQLVSNINACQVVAEAVRTTLGPRGMDKLVVDNRGKATISNDGATILKLLDVVHPAAKTLVDIARSQDAEVGDGTTSVTLLAAEFLKQLKPYVEEGLHPQTIIRAFRIATQLAVKKIKEIAVTIKKDDKQEQRRLLEKCAATALNSKLIAGQKDFFSKMVVDAVMMLDDLLPLKMIGVKKVQGGALEESQLVAGVAFKKTFSYAGFEMQPKRYMNPKIALLNIELELKAEKDNAEVRVNSMEDYQAIVDAEWNILYDKLEKIHKSGAKVVLSKLPIGDVATQYFAYRNLFCAARVVEENLKRTMMACGGSIQTSVGSLTDDVLGQCELFEEVQVGGERYNFFKGCPKAKTCTIILRGGAEQFMEETDRSLHDAIMIVRRAIKNDSIVAGGGAIEMELSKYLRDYSRTIPGKQQLLIGAYAKALEIIPRQLCDNAGFDATNILNKLRAKHAQGGMWYGVDVNNEDIADNFQACVWEPSIVRINALTAASEAACLILSVDETIKNPRSSVDGPPAAAGRGRGRGRPAHAH

SEQ ID NO: 127 - DNA, 1753 bp, Danio rerio
cttcctctttctgttacctggcaaaggggagcagcagctgaggagtgatctctcaatcttgaaacttatcaatcatgggaaaggaaaagacccacattaacatcgtggttattggccacgtcgactccggaaagtccaccaccaccggccatctgatctacaaatgcggtggaatcgacaagagaaccatcgagaagttcgagaaggaagccgctgagatgggcaagggctccttcaagtacgcctgggtgttggacaaactgaaggccgagcgtgagcgtggtatcaccattgacattgctctctggaaattcgagaccagcaaatactacgtcaccattattgatgcccctggacacagagacttcatcaagaacatgatcactggtacttctcaggctgactgtgctgtgctgattgttgctggtggtgtcggtgagtttgaggctggtatctccaagaacggacagacccgtgagcacgccctcctggctttcaccctgggagtgaaacagctgatcgttggagtcaacaagatggactccactgagcccccttacagccaggctcgttttgaggaaatcaccaaggaagtcagcgcatacatcaagaagatcggctacaaccctgccagtgttgccttcgtcccaatttcaggatggcacggtgacaacatgctggaggccagctcaaacatgggctggttcaagggatggaagattgagcgcaaggagggtaatgctagcggtactactcttcttgatgcccttgatgccattctgccccctagccgtcccaccgacaagcccctccgtctgccacttcaggatgtgtacaaaattggaggtattggaactgtacctgtgggtcgtgtggagactggtgtcctcaagcctggtatggttgtgaccttcgcccctgccaatgtaaccactgaggtcaagtctgttgagatgcaccacgagtctctgactgaggccactcctggtgacaacgttggcttcaacgttaagaacgtgtctgtcaaggacatccgtcgtggtaatgtggctggagacagcaagaacgacccacccatggaggctgccaacttcaacgctcaggtcatcatcctgaaccaccctggtcagatctctcagggttacgccccagtgctggattgccacactgctcacatcgcctgcaagtttgctgagctcaaggagaagatcgaccgtcgttctggcaagaagcttgaagacaaccccaaggctctcaaatccggagatgccgccattgttgagatggtccctggcaagcccatgtgtgtggagagcttctctacctaccctcctcttggtcgctttgctgtgcgtgacatgaggcagaccgttgctgtcggcgtcatcaagagcgttgagaagaaaatcggtggtgctggcaaggtcacaaagtctgcacagaaggctgccaagaccaagtgaatttccctcaatcacaccgttccaaaggttgcggcgtgttcttcccaacctcttggaatttctctaaacctgggcactctacttaaggactggataatgctgattaaaacccatcggaaaaattttcgcaggaaaggaaaacaacttggatttaagtgtggctccatttattgactgatagtgcctctttcagttattaaatttgtgttttgatggtttagaactgcacctgttgccacagtacaatttggaaacgctgatgaataaactaataaaggtattaaaaattg

SEQ ID NO: 128 - protein, 462 aa, Danio rerio
MGKEKTHINIVVIGHVDSGKSTTTGHLIYKCGGIDKRTIEKFEKEAAEMGKGSFKYAWVLDKLKAERERGITIDIALWKFETSKYYVTIIDAPGHRDFIKNMITGTSQADCAVLIVAGGVGEFEAGISKNGQTREHALLAFTLGVKQLIVGVNKMDSTEPPYSQARFEEITKEVSAYIKKIGYNPASVAFVPISGWHGDNMLEASSNMGWFKGWKIERKEGNASGTTLLDALDAILPPSRPTDKPLRLPLQDVYKIGGIGTVPVGRVETGVLKPGMVVTFAPANVTTEVKSVEMHHESLTEATPGDNVGFNVKNVSVKDIRRGNVAGDSKNDPPMEAANFNAQVIILNHPGQISQGYAPVLDCHTAHIACKFAELKEKIDRRSGKKLEDNPKALKSGDAAIVEMVPGKPMCVESFSTYPPLGRFAVRDMRQTVAVGVIKSVEKKIGGAGKVTKSAQKAAKTK

SEQ ID NO: 129 - DNA, 1891 bp, Danio rerio
acgcggggatcccataatgcacctcgtcgctgtcatggctgctcactgatgacacttgcggcacatgggcagaaatctaacacattacagtgaacacacgaacctcaacacgagtctcacactcacacagagagcaggtcctgagcgacgcgacgctgtttcattctccgcggaaccgggcacacaaggagcgcgatgctgcagctcgaggcggcggttctgggcggattgagtctggactggaaacagagcagcttcagcaccgcagatgcaggttcattgctgcagttggtatatgagggagattttgaggccgtcctcttcagctctgcggtgcaggggcttctgggagtttgtcctgaagagggtgacagcattgaggcctatctggagagacaggtgctgtcttacctgagtgatgccaccgaggagcagaggagccacagggaagttgctctgttggcggtggcagtggggtgtctgaaccttttcgcccagagtaactggaccggcccaccagttgagctccatgtctctgatgttcttcctgaagccctgctccagaacttttctcagcctgcggccctgagtacagctctgttgtccagtcttcagctggatggagagtctgtgtacagtctggtctccaaccctctcctgctgctgctgaccagagtcatcttcgtcaactgcgccgccaaactggagagccttcagctgctgccctggtggacgctgcgatatgtcgctctccatcagcagatactggaggaaagatccccacagctcctcaacctgatgctcagctgcatcgagaaagtgtgtaaatgtgacgagctgttcagcagtaatgcacacagaaacctggccattcagttccacctggagtgcagttatatctgcctgacctactatgagtatcacaaggccaaagaacatctgcagacggctcgtgatctgtcaggactcgatgttaacatgacaggtgctctagggaagcgaactcgttttcaggagaactttttggctcagctcatcttagatgtaaagcgaaaggacaactcgcccttttacaactccgagagtccctccagtacaccaacacctaaagagctcctaccaaaggaccatcagctgagcgacgatacagtcctaaaccagattaatcttgcagaaccatctgagcatgaacttccagacctaagcgctgaggagcaagccttcatgctggccacatgcacagactttcggaagaataatccagttcacaagctgaatgatgaggagctcctggccttcaccacggtaagacacattgttattctctaacaggccgcagtgcagttgacacatctcttatcagcacacacagttgtgttttcaggttgacagatggagggcatgcaaacaggtctctctcaattgctcacagtttagcacctagaatgtgtgtgtttgctctaacaaatgcaaacaaaggaaaatggtctgtggcttctgctgttgtgctccacaaaaatgaaaagatgtcaatctctggtcatttctgctgctctgctcaaatctacaatgtatagttcttctcaaaagttgacatgcccttcgcaaaatctccaagatgattatcatttttaaataagcaaacatgagcattaggaaaagttgatcgactatctctcatgccaagtgctgtcggaattttaaatggaaaccattggtttatttttttatacatttatattatatgatgaatttgagacaattttccacagtatgaataataaagtaattattaaatgcaaattttactttttttttcttcttcaattttgtgctttataataattatagcaccgacacaacagcaaaaaaaaaaaaaaaaaaaaaaaaa

SEQ ID NO: 130 - protein, 377 aa, Danio rerio
MLQLEAAVLGGLSLDWKQSSFSTADAGSLLQLVYEGDFEAVLFSSAVQGLLGVCPEEGDSIEAYLERQVLSYLSDATEEQRSHREVALLAVAVGCLNLFAQSNWTGPPVELHVSDVLPEALLQNFSQPAALSTALLSSLQLDGESVYSLVSNPLLLLLTRVIFVNCAAKLESLQLLPWWTLRYVALHQQILEERSPQLLNLMLSCIEKVCKCDELFSSNAHRNLAIQFHLECSYICLTYYEYHKAKEHLQTARDLSGLDVNMTGALGKRTRFQENFLAQLILDVKRKDNSPFYNSESPSSTPTPKELLPKDHQLSDDTVLNQINLAEPSEHELPDLSAEEQAFMLATCTDFRKNNPVHKLNDEELLAFTTVRHIVIL

SEQ ID NO: 131 - DNA, 565 bp, Danio rerio
acgcgggagcagcggtaacaacgcagagtacgcggggctctttcccgtctgaaccgccgccatgaaggtcgagctgtgcagttttagcggatacaaaatctatcccggtcacggccggcgatacgccagggttgacggaaaggttttccagttcctcaatgccaaatgtgagtctgcgtttctgtccaagaggaacccgaggcagatcaactggaccgttctgtaccgccgcaagcacaagaagggccagtctgaggaggtgtcgaagaagcgcacacgtcgtgcagtaaagttccagcgggcgatcaccggcgcttctctggccgagattctggccaagaggaaccagaagcctgaagtgcgtaaagcccagcgcgagcaggccatcagggctgcaaaggaggcgaagaaagccaagcaggccaccaagaagcagacgacccagagcagcaaggctcctgctaaatcagctcagaaacagaagattgccaagcccatgaaggtcagcgctcctcgcgtcggtggcaaacgctaaactctgatgtgctaataaagattcactgct

SEQ ID NO: 132 - protein, 157 aa, Danio rerio
MKVELCSFSGYKIYPGHGRRYARVDGKVFQFLNAKCESAFLSKRNPRQINWTVLYRRKHKKGQSEEVSKKRTRRAVKFQRAITGASLAEILAKRNQKPEVRKAQREQAIRAAKEAKKAKQATKKQTTQSSKAPAKSAQKQKIAKPMKVSAPRVGGKR

SEQ ID NO: 133 - DNA, 1735 bp, Danio rerio
gcagcggacagcacaatgtctaatggtcaggaaacggcgtgtctaaacggcgaaccggcgctcaaacgctccagagacagtctggattcactgcgggtcccgcgggagccccagaataaagtgaccgtagtgctgggcgcgcagtggggcgatgaggggaaggggaaagtggtcgacctcttggcaatggacgccgacattgtatgcagatgccagggtggaaatagcgcgggacacactgtggtcgtggactcggtcgagtatgacttccacctgctgcccagcggagttcttaacaaaaaggccacttcctttattggaaatggtgttgtgatacacctgccaggactctttgacgaggctgagaagaacttgcagaagggcaatggacttcaaggatgggaggaacgactgaagatatctgaccgggcacacattgtgttcaacttccaccaggctgttgatggcatacaggagcagctcagacaacagcaggcaggaaaaaatttgggcacaactaagaagggtattggacctgcatactcttccaaagcagcacgtaatggactgagagtgtgtgatctggtctccgacttctctgtctttgaagaaaagtttcgggtgctggctggacattatcagaccacttatcctaatcttaacattgacattgatgctgagcttcagcagctcaagaattttgctgaaagattacggccactagtgactgatggggtttatttcatgcacaaagccctcactggaccaagcaagaagattctggtggaaggagccaatgctgctttactggatattgattttgggacctacccctttgtgacctcatcaaactgcactgttggaggcgtctgcacaggtcttggcatccccccatctcatgttggccgtgtgtatggagtggtgaaggcctacaccaccagggttggagtcggggcatttcccactgagcagaacaatgaaattggagatctgctgcagagcagagggagggaatttggagttacaacaggcaggcggcggcgctgtgggtggctggatctggttttggttcgctatgcccacatggttaacgggttttcagccattgcgctgactaagttggacattcttgacacattgcctgaaataaagattggcacagcctacacagttgatggaaagcctcttcccagttttcctgcaaacatggacatgctcacaaaggttcaggtgacttatgagacgttccctggctggagttgtagcactgagggagtgcgcagcttcgatgagctgcctacacaggcacaaagctacattaaatttattgaggattttctgcaagtgccagtgaagtgggttggagttggcaagtcaagagaaagcatgatcaagctgttttgacgagggcgaagggacaggctgagctgatggtcatcgactggtctggcacgacatttatcatcatgaccgctggagcacctgaactggcttttatgcatcagcgctgagatgaagactaaccgtgggtttagaatccactgaatgtcccactcaaagtgaacaccagcattgtgtataggggtcagatttgagcaccagacctgcttcagtgcatttgtaattgcttgattcgttcctgtttgtttccaaagcgcatcccaaacctgctcaaaacatacctctgctaatcagtaatagtctgccggtaaaagtaatgagctcccaactgaaa

SEQ ID NO: 134 - protein, 462 aa, Danio rerio
MSNGQETACLNGEPALKRSRDSLDSLRVPREPQNKVTVVLGAQWGDEGKGKVVDLLAMDADIVCRCQGGNSAGHTVVVDSVEYDFHLLPSGVLNKKATSFIGNGVVIHLPGLFDEAEKNLQKGNGLQGWEERLKISDRAHIVFNFHQAVDGIQEQLRQQQAGKNLGTTKKGIGPAYSSKAARNGLRVCDLVSDFSVFEEKFRVLAGHYQTTYPNLNIDIDAELQQLKNFAERLRPLVTDGVYFMHKALTGPSKKILVEGANAALLDIDFGTYPFVTSSNCTVGGVCTGLGIPPSHVGRVYGVVKAYTTRVGVGAFPTEQNNEIGDLLQSRGREFGVTTGRRRRCGWLDLVLVRYAHMVNGFSAIALTKLDILDTLPEIKIGTAYTVDGKPLPSFPANMDMLTKVQVTYETFPGWSCSTEGVRSFDELPTQAQSYIKFIEDFLQVPVKWVGVGKSRESMIKLF

SEQ ID NO: 135 - DNA, 730 bp, Danio rerio
gtcgtgctgatttaaacgcgtccagcttcacttttcctcaattaatttgcttttttcacaatgtccataaaaggtaaatgctcttttcagcgacttctatgttgatgtgagccagtacagggatcagcattttaagggcaaccgttacgagcaggagaagctcctcaaacagagcgccacgctgtatgtaggaaacctgtccttctacaccacagaggagcaggtgcatgtgctcttcgccaagtgtggagacgtcaagaggatcatcatcggcctcgacaaaatcaagaagactgcgtgcggcttctgcttcgtcgaatattacactcgtgcggacgctgagaacgccatgaggtttgtcaacggcacgcgtcttgacgaccgcatcatcagaacagactgggacgcaggcttcaaagagggccgacagtacggacgggggaaatctggtggtcaggtcagagatgaatacaggcaggactatgatcccgccagagggggttatgggaagatggtgcagaaatcttgaacatccctttcgtcattttccttttctttttttttaatcttttaaaatatttccacgttatgaatgaatctggcaggattacggctgttttctgaatgtacatatcggagtttgctttaggaatcgctcaagttcttctggtccagcatgatgagtttaataaattgtgttttgtttcccaaaaaaaaaaaaaaaaaaaaa

SEQ ID NO: 136 - protein, 145 aa, Danio rerio
DFYVDVSQYRDQHFKGNRYEQEKLLKQSATLYVGNLSFYTTEEQVHVLFAKCGDVKRIIIGLDKIKKTACGFCFVEYYTRADAENAMRFVNGTRLDDRIIRTDWDAGFKEGRQYGRGKSGGQVRDEYRQDYDPARGGYGKMVQKS

SEQ ID NO: 137 - DNA, 2106 bp, Danio rerio
gcaggtcacgctagcccgtcgagccaggagaaatacagcatttttcatttccagcactctggtctaatgggaacactctttgtaaatgcgttttaagattcttgcttgagagaagtacacaccggccctggccttgaggcttatctgaaaggtttcaacttcactggtgaacctccttggctgtctgacatctgttagccacgtttcaatcccatctcttccattcgtctgtttcttttgcaatcaaatcttgaacaaacactatggcggcttgcaccggatcagactttgacttcgccttcctggaggagggattctgtgcgcgcgatatcgtggagcagaagatcaatgagtcctcgctctcagatgataaagatgctttctatgtggcggatctgggtgatgtcctgaagaagcacctccgctggctgcgtgttttgccccgcatcacgccgttttacgcagtgaagtgcaacgatagcagggcggtggtcaccacactggcgtctctgggagccgggttcgactgtgccagcaaaacggagatccagatcgtgcagtcagtgggtgtggatcccagcaggatcatctacgccaacccgtgcaagcaggtttcgcagatcaaatacgcctctgctcacggcgtgcagatgatgacgttcgacagcgaggtggagctcatgaaggtggcccgtagccacgaaaatgccaagctggttctccgcatcgccacagacgactcgaaggctgtttgcaggctgagtgtgaagtttggagcgacgctgaagagcagccggctgctgctggagagagcaaaagagctcgggctggacgtgatcggggtcagtttccatgtgggcagcggctgtacggacccggaaacatacagccaggccatctcagatgctcgctatgttttcgacattggggcggagctgggctataacatgagcctgctggacatcggcggaggatttccaggctctgaggacaccaaactcaagtttgaagagattgctgctgttattaaccctgcgctggataaatacttccctgtggactctggcgtgaggatcatcgctgaacccggccgttattatgtagcatctgcttacacactggcggtcaacatcatcgccaaaaaggtcatcatgaaggagcagtcagcctccgatgaagaagaggatgggtccaatgaccgaaccctgatgtactacgtgaatgacggcgtttatggctccttcaactgcatcctgtatgatcatgcgcacgtgttgccaactctgcacaagaaacccaagcccgatgagcgcatgtacccctgcagtatctggggcccgacctgcgacggtctggaccgcattgtggagcagtgcagcctgccggacatgcaggtgggcgactggctgctgttcgagaacatgggcgcctacaccgtcgccgcatcctccaccttcaatggcttcaagaaaccgatattttattacatcatgtcccgaacagcctggcagtgcatgcagcagattcgtgcccagcggattcctgcacttccattggaggagccgagcgctggaaacgtgccatcccactgcgggcgcgagagcagtctggatgttcccgccaaaccctgcccgactcaagtgctgtagcagaagacgctcaaccaaacctgctctcgggtccagctaatgcagatctgtgttactgatctccaacttctgatctaaacgcctcagagctgaggagaaaacaaacgcacagtccaattcttatttcctgttatctttgttttaactggtttttgttgattgtctgtatttgtatttcgaggccagatgcttgagcgaggacttcttcctcttcctcagtgaccatttccattttatacttgattagtaacttaattttttaatattgagttgaagtctttgtattataattattcagagctgagcttgtatgccactggtgcatttgaaacggacgtgaaagagatgggattgcatctattatgctttcctatggaaactgctttttgttttttttaagtattacgtttttttgtatttttatataaataaagatgctgtaaaatggcaaaaaaaa

SEQ ID NO: 138 - protein, 461 aa, Danio rerio
MAACTGSDFDFAFLEEGFCARDIVEQKINESSLSDDKDAFYVADLGDVLKKHLRWLRVLPRITPFYAVKCNDSRAVVTTLASLGAGFDCASKTEIQIVQSVGVDPSRIIYANPCKQVSQIKYASAHGVQMMTFDSEVELMKVARSHENAKLVLRIATDDSKAVCRLSVKFGATLKSSRLLLERAKELGLDVIGVSFHVGSGCTDPETYSQAISDARYVFDIGAELGYNMSLLDIGGGFPGSEDTKLKFEEIAAVINPALDKYFPVDSGVRIIAEPGRYYVASAYTLAVNIIAKKVIMKEQSASDEEEDGSNDRTLMYYVNDGVYGSFNCILYDHAHVLPTLHKKPKPDERMYPCSIWGPTCDGLDRIVEQCSLPDMQVGDWLLFENMGAYTVAASSTFNGFKKPIFYYIMSRTAWQCMQQIRAQRIPALPLEEPSAGNVPSHCGRESSLDVPAKPCPTQVL

SEQ ID NO: 139 - DNA, 2043 bp, Danio rerio
ctgagtcggtctcagtgaagatctggcgtcgatgatttttcaactgtgtacgaagagagggccccaaaatctcttacttttttctactcaaataacggacattcgcttactaccttttgtgctccatatggttcatggaaggaaaatctcaatttcagccaccacaatcccgcccattatgccatttttcagccaataatgcttaagatcatgacagtttacgttctcttaaactgttataaagctgaaatttgttgctgttaagcacaaaatattaatagcaacccgagacgcagaggagaggtattaatataagcagccatggccgtgggcccagtggaccccagagaggtgttgaaaggagttgaagcactgctggggaaggatggagatctacgtagttttgagggagtcgctaaagtcttcagtcttatgaaggcctcacataatatggtcatcagatgcatgtatctgaacatatcacttgagacaaaatcacatgatgtgctaattcgattcattcgagttggaggctacaagttgttgaactcttggctcacctactcgaagaccaccaccaacacgcctctgttgcagctcattctgctgaccctgcagaagcttcctcttactgtggaccacctcaaacagaataacactgccaagctggtgaaacatctgagcaagagcggagagacagaagagctgaggaagttggcatctgtgctggtggatggctggatggctattattcgctctcagagcgtctccagtggagcatcgcctaatgacaagaaacggaaaaaggaggatggaaaagtgcgtccagaggtgaagccaaaggaaaaggcagctgaggaggagaagaagaaggacaaaccaaaagcttatgctccaagtcatgctaagatccgctctacaggtttggaggtggagagtccggctccagttcctgtgaagaagccacccgttgcaccccaacttggagacaaatacaacatcaagccagctgtacttaaaagaccaagcacatctttgactgacgcaccaccagtggaaaagaagtacaagccacttaacaccacccccaactcaaccaaagagatcaaagtcaaactcatacccccacagccgatggaggccactggatttttggacgccctcaactctgctccagtgcctggcatcaagatcaagaaaaagaaaccaaaagcagtttcacccaccagtaataagccctgtccatttgacagcaggccgcagtcattttcaggcgcacaggccaaaccatcttcccctgaaacgccttcatctcaaacccctcctcatgagaatcaagaactggagcagcctggcacacccgtgcccaccgaggagcctgaagccatggaaaccggtgataaacccaatgttctggatgaacctcgcagtgaggaggagattctgattaagaaaggaaagaagaagaacagcgtgcggtgggcgcaagaggatcatctcaaagagtacttctatttcgatctggatgaaactgagagagtgaatgtgaacaaaattaaggactttggtgaggcggctaagcgggagctgatgatggataggcagacgtttgaaatggctcgtcgtctttctcatgacaccatggaggagagggtgccctggaccccaccgcgacccttgaccctattaggctgcctggtcactgcaggtgccagcagccgagagaagatgatccagagagaccgagagatgggcattctgcaggagatcttcctctgcaaggagagtgttccagacagtcctcatgagccagatcctgagccgtatgagcccatgcctcctcgtctcatcccactggacgaggatagcagtatagtagaggacatggattccactgatcctgcctctactcccggctctaatgaaggctccaagctgcctcctgtatttgttaaccttatggggagcctgggcaacagcggccgcatcccgcaggcacagggcaatgccccacagccaaaaaaaaaaaaaaaaaaaaaaaaa

SEQ ID NO: 140 - protein, 574 aa, Danio rerio
MAVGPVDPREVLKGVEALLGKDGDLRSFEGVAKVFSLMKASHNMVIRCMYLNISLETKSHDVLIRFIRVGGYKLLNSWLTYSKTTTNTPLLQLILLTLQKLPLTVDHLKQNNTAKLVKHLSKSGETEELRKLASVLVDGWMAIIRSQSVSSGASPNDKKRKKEDGKVRPEVKPKEKAAEEEKKKDKPKAYAPSHAKIRSTGLEVESPAPVPVKKPPVAPQLGDKYNIKPAVLKRPSTSLTDAPPVEKKYKPLNTTPNSTKEIKVKLIPPQPMEATGFLDALNSAPVPGIKIKKKKPKAVSPTSNKPCPFDSRPQSFSGAQAKPSSPETPSSQTPPHENQELEQPGTPVPTEEPEAMETGDKPNVLDEPRSEEEILIKKGKKKNSVRWAQEDHLKEYFYFDLDETERVNVNKIKDFGEAAKRELMMDRQTFEMARRLSHDTMEERVPWTPPRPLTLLGCLVTAGASSREKMIQRDREMGILQEIFLCKESVPDSPHEPDPEPYEPMPPRLIPLDEDSSIVEDMDSTDPASTPGSNEGSKLPPVFVNLMGSLGNSGRIPQAQGNAPQPKKKKKKKK

SEQ ID NO: 141 - DNA, 1254 bp, Danio rerio
ggcagggtagcatgtggaggtcagagatgtagaaacgcttcgtcagagaataaaaactttaattattagacatcagcgtttgataactcgacatagtgtcatctatgcggatttacatcagatattattgaacacaaggctgtcggtaaagagattttgtgtggatagtctccatcagtttgacgcgggtcagtaaggaagaattgtaatcagcgaagcggtattaactgtataagtgaatctccgaaaacatggataacaacacaccgccaccgggaggatttaaaggaggactcgggagcatttttggtggaggaacccctgagtattcaaacactgagctttcaggagtaccgttgactggaatgagtcctctttccccatacctcaatgttgaccctcgttacctcatacaggacactgatgagttcatcttgccaactggggcaaataaaactcgtggtcgctttgagctggccttcttcaccattggtggatgttgtataacaggagctgcttttgggacactcaatggtctccgtatgggcctgtcagaaactagagacatgccatggtcaaaacccagaaatgtacagattttgaacatggtcacacgacaaggggcatcgtgggccaacacactaggatcagtagctcttttgtatagcgtgtttggtgtggccattgagagggccaggggtgcagaggacgacctcaacacagtggcagctggaacattaaccgggatggtatttaaatccacaggtggactcaaaggagttgccagaggaggtctcattggtttagccatgtcagggctgtatgctctgtacaacaactgggatcacctaaaggggaaatctccttcacattactgagaatgactgagaactcaaaccatggacttattttgactctgagccaattcttggtatttattgcccccctttggagatccattcatttctacaagatacaaaaggacccccacatgtgcacagattcttcaaagttaggttaagtaaactatttaaatggaaataccaaactgcctccaaatatgtggactatgttgcaatgagttagctggtaacattgtgttcgttcatgtatgtttttgttgatgttaagttgcatctggaggaactttatgctttgattacgaggttttatctgttctcattgtggatttatgcgttttttcagtgctgattaaactgggctcttcctctttagaaaaaaaaaaaaaaaaaa

SEQ ID NO: 142 - protein, 208 aa, Danio rerio
MDNNTPPPGGFKGGLGSIFGGGTPEYSNTELSGVPLTGMSPLSPYLNVDPRYLIQDTDEFILPTGANKTRGRFELAFFTIGGCCITGAAFGTLNGLRMGLSETRDMPWSKPRNVQILNMVTRQGASWANTLGSVALLYSVFGVAIERARGAEDDLNTVAAGTLTGMVFKSTGGLKGVARGGLIGLAMSGLYALYNNWDHLKGKSPSHY

SEQ ID NO: 143 - DNA, 2850 bp, Danio rerio
cgccgagacgcgcgcacacgtgtctcactagcgtctgcggtgttttcatcgcacatttctttaatttcctcgattatctaccatccgtgagagcttcagaaatggcccagagaaagaaaaagctgatgaggaggaagaaaagcggcgcaaaccgcgatcacgagctccagtccgacgatgaagagtttgaggtggccgcaggagtcaaagatgacgaaaataccacggtcaggaggcttcctcggtttccggcctcgtctgagtgcgtgtctgacgtggagctggacaccagagagctggtcagagctcagaacaagaagaagaagaaatctggaggctttcagtctatgggtctcagctatccggtgtataaaggcatcatgaagaagggctataaagtgcccacacccatccagaggaagacgatcccggtgattctggacggtaaggatgtggtggcgatggcccgcaccggcagcggtaagactgcagcgttcctcgttcccctgtttgagaagctgaaggctccgcaggcacagacgggggctcgggcactgatcctcacacccacccgagagctcgccctgcagaccatgaagttcaccaaagagctggggaaattcactggtctgagaacagcgctcattctcggtggagacagcatggatgatcagttcgctgctcttcacgagaacccagacatcatcatcggtactcctggtcgtctgatgcacgtcatccaggagatgaacctgaagctgcagtcggtggagtatgtggtgttcgacgaggccgacagactgtttgagatgggctttgcggagcagcttcaggagatcatcagacgtcttccagacgcccggcagacgctgctgttctccgctacgcttcccaaactcatcgtggagttcgccagagccggtctgacggagccggtcctcattcgtctggatgtggacactaaactgagtgaacagctgaagctgtcgttcttctctctgcgtctggatgataagccggctcttctgctgcacctgctgaggaacgtggtgaagccgcaggagcagacggtggtgttcgtggccactaaacatcatgtggagtatctgaaggagctgctgtcagcggagggtgtggactgctcctgcatctacagtgctctggatcagacggctcgtaagatcagcatcggtcgctttgttcatcgtaaggtgatgctgctgctggtgacggatgtggcggcgcgcggtatcgacatccccctgctggacaacgtcatcaactacaacttcccctgcaaacccaagctcttcctgcacagagtcggtcgtgtggctcgcgctggtcgagggggaacagcatacagtctggtgtgtcctgatgaggtgccttacctgtatgacctgcacctgtttctgggtcgacccatgcagctcgcacaccccgaacacacacaagaagcggacggtgtgttcggccgtgttcctcagagtgttctggatgatgaggagtgtcagctgatcacggcgcatcagaactctctggacctgcagaatctcagacgcgtgtcagaaaacgcctacaagcagtacctgaagtccagacccgtcccgtctgcagagtccatcaagcgcagccgcaacacacagctcacagacatggcggtacacccgctgctgggctgtgggctggagaagatggaactggatcgactgctgatggtcgacaccatcaagggctacaaggccaaatctactatttttgagatcaactccagcaataagacgagcgctagcgaggtgatgcgcgccaaacgctcccgggaccgccagcgggtggagaagttcagcagagcaagagcagagctgagggcggagccaggggcgggactgagggcggagccaccagtgcacagagagacacaggaggaagacgaggaggagcaggaggagctgtcgacggtgttctctgaggtggttggaggtaagagacggaggccggatgcggagccgcacacacacaagagcaagaagagcagaacagcgggccgagaccaggagttctacatcccatacagacccaaagacttcaactcagagcgcgggctcagtctggactctggtgcgggctcgtttgagcagcaggcctcctctgcagttctggatctgattggagatgagagcaacacattaaaccagcacaagagcctgatgaagtgggaccgcaagaagaagcgcttcgtccgtgacaccggcaaagaggacaaacacaagaagatcaggacggagagcggacaactgatcagaggacacaagaagaagaagagcttttatgaagagtggaagaagaagtataaggtggagggcggagcttcagactctgatggagaagggggaggagctcagagggggcggggctctgcaggtcgtcggggtcgtggtcggggtcgctctgcagcaggttcccaggctacgccagcgcagcatcagggcggttcaggtgttcgctcggagctgaagaaccggcagcagatcctgaagcagcgcaagaggaaagcaaagcagcagttcctgcagagcggaggcatgaagaagctcagaaacaagggaaaacagcgcctccgagaggtcatgaagtccggcttcggccgcggagcagtcaagaagggcaagatgaggaagaaactttaggagaatgcatcgtgtgtgtgtgtgtgtgtgtgtgtgtgtgtgcgtgtgtgtgtgtgtgtgtgtgtgtgtgtgtgtgtgtgtgtgcgtgtgtgtgtatgtttataagaataatatggagatcagtgagtgtgtgtcatctctgcgtctgcattaaaggtgt

SEQ ID NO: 144 - protein, 862 aa, Danio rerio
MAQRKKKLMRRKKSGANRDHELQSDDEEFEVAAGVKDDENTTVRRLPRFPASSECVSDVELDTRELVRAQNKKKKKSGGFQSMGLSYPVYKGIMKKGYKVPTPIQRKTIPVILDGKDVVAMARTGSGKTAAFLVPLFEKLKAPQAQTGARALILTPTRELALQTMKFTKELGKFTGLRTALILGGDSMDDQFAALHENPDIIIGTPGRLMHVIQEMNLKLQSVEYVVFDEADRLFEMGFAEQLQEIIRRLPDARQTLLFSATLPKLIVEFARAGLTEPVLIRLDVDTKLSEQLKLSFFSLRLDDKPALLLHLLRNVVKPQEQTVVFVATKHHVEYLKELLSAEGVDCSCIYSALDQTARKISIGRFVHRKVMLLLVTDVAARGIDIPLLDNVINYNFPCKPKLFLHRVGRVARAGRGGTAYSLVCPDEVPYLYDLHLFLGRPMQLAHPEHTQEADGVFGRVPQSVLDDEECQLITAHQNSLDLQNLRRVSENAYKQYLKSRPVPSAESIKRSRNTQLTDMAVHPLLGCGLEKMELDRLLMVDTIKGYKAKSTIFEINSSNKTSASEVMRAKRSRDRQRVEKFSRARAELRAEPGAGLRAEPPVHRETQEEDEEEQEELSTVFSEVVGGKRRRPDAEPHTHKSKKSRTAGRDQEFYIPYRPKDFNSERGLSLDSGAGSFEQQASSAVLDLIGDESNTLNQHKSLMKWDRKKKRFVRDTGKEDKHKKIRTESGQLIRGHKKKKSFYEEWKKKYKVEGGASDSDGEGGGAQRGRGSAGRRGRGRGRSAAGSQATPAQHQGGSGVRSELKNRQQILKQRKRKAKQQFLQSGGMKKLRNKGKQRLREVMKSGFGRGAVKKGKMRKKL

SEQ ID NO: 145 - DNA, 2407 bp, Danio rerio
ccacgcgtccgcccacgcgtccgcccacgcgtccggctccgtgtggacagtcgtgcgctggcgggagtttgccggattaaagccgtttaaaacgaagaaatcgcaaagctttcctgaaactcggccttcactgaaacacaatgggcgacagtgatgacgagtacgaccgcagacgcagagacaagttccgccgggagaggagcgactatgaccggtccagggagagagaggaccgccgtagggatgactggaatgaccggcggccctcggctcgggaatgggaccggggccgagagcgtcgcagcagaggggagtaccgggactacgacaggggccgacgagagagattctcccctccacgacatgacatgagcccccaacagaagcgcatgaggagagactgggatgaccatggaggtgatccgtaccatggtggctatgatctgggttatggtggcggtggaggacccagctacgcccctcctcagccgtggggtcaccctgacatgcacctgatgcagccccatcatggaatccccatccaggccaggctgggaaacatccacgatatggatcttggtcctcctcctcctgtgatgaagacctttaaggagtttctgatttctctggatgactctgtggacgagacggagtcggtcaaacgctacaatgagtacaagattgattttaggcgtcagcagatgcaggacttcttcctggcccataaagatgaagagtggtttcgctcaaagtaccacccggatgaggctgggaggcgtaaagttgaggcccacagtgctctgcagaaccggctgggcgtctacatgtacctgatggacaacaactggtttgagtcggtctctctggatatcgagcgtgctccgcaaatcaccaagattttagatgcagccgtgataaagatggagggaggagcagaaaatgacctgcgcatcctggagcagcccagcgaggaagaagaggagcgtgagagactgtcatctggtggaactccctctgaaccctctaaacgggacgaacccaagcctgctgatactgagaacaaaccctcagaggaaaaagacaagatggaagagggtgaggagagcgctgagaaagacggagaaaaggcttctgcaggaggaagtgaaggagaggagaaaactgagaaggaagcacctgctgaaccaatccctgaacccaagaagctgagtaagaagaggaagaggaagcacagtggagacagcgaggatgaagccagtgcatctgagagcgaatctgattctgactccgactcaaactctcactgctcggagaaaccctcggagagagagagagaaccagaagaagtggaggaaaaggaagaagaggaagaagaggagggtgaagcggctgatggaaaggagcagacggatgagcagacggaaagagagaaggaaaaagagaagagagtcaaggatgatcagcctccacgtcctcgacctcttcacaggacgtgttctctgttcatgagagagtatcgctccaacaatatctaaagcagaaggtgagtgagtggcagaagacttttgaagagaaaatgggacccttgttcagtgtgaaagaaaacctgtctgaggaagaagcggagaaaatgggccgcaaagacccagaacaggaagtggaaaagtttgtggtggccaacacgcaggaaatgggcaaagacaagtggatctgccctgtaagtggcaaaaagtttaagggcccagagtttgtgaggaagcacatcctgaacaaacacggggacaagattgaggaagtaaagaaagaggtggtgtttttcaacaacttcctaatggatgctaaaagaccatgcatcccggagatgaaacccccaccccatccaggcccaggacagggagtgctgtcacccggtggtcttcccttcccaccgcagggtcctcaaggtctcatgggttttggtcagcccagacctccacttatgggttatggaggtggtcctccgtatccccctaaccagtatggtggaggcagaggcaactatgataacttccgtggacagggcggatatcctggaaaacccagaaacagccgagtgatgcgtggagatcctcgcaatatcatcgagtatcgtgatttagacgcacctgatgatgtggatttcttttagagctccagttttagtctctacattttaacctgttttttttttacggttgtcacagcattttctcgttttttgcattcattccagaagctgcccgccgccgtctcctttcacatgtattacagtagcaaagcttcactttgtgttcaatgtcattatgtacttcatgtcataatgtgagcgtgtgctttatggcctttttttcctccaataaaactttaatttggatgcaaaaaaaaaaaa

SEQ ID NO: 146 - protein, 673 aa, Danio rerio
MGDSDDEYDRRRRDKFRRERSDYDRSREREDRRRDDWNDRRPSAREWDRGRERRSRGEYRDYDRGRRERFSPPRHDMSPQQKRMRRDWDDHGGDPYHGGYDLGYGGGGGPSYAPPQPWGHPDMHLMQPHHGIPIQARLGNIHDMDLGPPPPVMKTFKEFLISLDDSVDETESVKRYNEYKIDFRRQQMQDFFLAHKDEEWFRSKYHPDEAGRRKVEAHSALQNRLGVYMYLMDNNWFESVSLDIERAPQITKILDAAVIKMEGGAENDLRILEQPSEEEEERERLSSGGTPSEPSKRDEPKPADTENKPSEEKDKMEEGEESAEKDGEKASAGGSEGEEKTEKEAPAEPIPEPKKLSKKRKRKHSGDSEDEASASESESDSDSDSNSHCSEKPSEREREPEEVEEKEEEEEEEGEAADGKEQTDEQTEREKEKEKRVKDDQPPRPRPLHRTCSLFMREYRSNNIQKVSEWQKTFEEKMGPLFSVKENLSEEEAEKMGRKDPEQEVEKFVVANTQEMGKDKWICPVSGKKFKGPEFVRKHILNKHGDKIEEVKKEVVFFNNFLMDAKRPCIPEMKPPPHPGPGQGVLSPGGLPFPPQGPQGLMGFGQPRPPLMGYGGGPPYPPNQYGGGRGNYDNFRGQGGYPGKPRNSRVMRGDPRNIIEYRDLDAPDDVDFF

SEQ ID NO: 147 - DNA, 2498 bp, Danio rerio
gggcactgttggcctactggcttttctctctcacccgggcggcggagcgcaccggtagcgctaattatccgtttacaactcactgggactccagacagagaccgtgatttcaggggaccgtcatcatggcgattaagttcttggaggtaataaaaccgttttgtgcggttttgcctgaaatccagaaaccagaacgaaagatccagttcagagaaaaggtgctatggactgccatcacattgttcatctttcttgtttgctgtcagatcccactgtttgggatcatgtcttcagactcagcagatccattctactggatgagagtcatcttggcttccaacagaggtactttgatggagttgggtatctctcccattgtgacctctggtctgatcatgcagctgctggctggagctaaaatcattgaggtcggagacacaccgaaagacagagcgctctttaatggagcccaaaaattgtttggtatgatcatcaccattggccaggctattgtgtacgtcatgactggaatgtatggagacccttcagagatgggtgctggcatctgtctgctgatcatcattcagttgtttgtggctggtctgatcgtgttgctgctggatgagttgctgcagaaaggttatggtttgggctctggtatctctctcttcattgccactaacatctgtgagactatcgtatggaaagcattcagcccaaccacagtcaacacaggcagaggtactgagtttgagggagccattattgctctgttccaccttttggccactcgcactgataaagtcagagctctgagagaggctttctacagacagaacctgcctaacctcatgaacctcatcgctacagtcttcgtctttgctgtggtcatatacttccagggcttcagagttgatctgcccatcaagtctgcacgttaccgtggccaatacaacacatatcccatcaagctgttctatacctccaacattcccatcatcctgcagtctgctctggtttccaatctttacgtcatctctcagatgctctccactcaattcagcggcaacttcctagtcaaccttctggggacttggtctgacacctcatccggtggtccagcgcgcgcctatcctgtgggcggtctctgttactatctgtctcctccggagtcgtttggttctgtgcttgatgacccagttcatgcagtgatctacattgtgttcatgctgggatcctgtgccttcttttcaaagacctggattgaagtttctggatcttctgccaaagatgtagctaaacagctgaaagaacagcacatggtgatgaggggacacactgggcgccatcggctccggaacgggaatcctattggctgttaccatcatctatcagtactttgagatatttgttaaggaacagagtgaagtcggcagcatgggagcgctactcttctaaacaaggacactcacaaacacacacatacttgctgacacctttttattatttttatgattgttgttgtcatttaatttttttatttcggtgcaatttgagcataattagtggtctgtcacgagcgctgtcttgggagaacgttctcttagccgctagctcaggccgagtaagttctcctgtatgtcagaagtggcaaaacgtttctaaaattcagtgatgaagctgaacttcagtaatggaacagtaaagatggactctgtgagctctgactaggaatatttagatgaattaaattttctcttcacatcagtctgtccttcatctgtctccagccatcaccgaatccattcatccactcttttctttagttcctgcctggtcctagttgataaagattttccagagagtattgcttatattgggtgactgatttactcgacactgccaattttgacaactgcttcgtgtttgtcagtcatatcggtcttgtatgtaaagctggcaggcagtttgcttttctaactcattgaatcatgctaagtcataaaaactgtgatttagttatggcttcaaatataagagctaaaccacaggttcttgttttccagtcagcaattgtcaattttaaacaaggcagcataggagtaaaaccacgcacaatttggccacacatgtgtgtgtggtggtttttctgtgtttgttgaacaagtatttcaaaacgctgggatctttttttttttttttttttttgtacccaacacatcttacacaatagagagcaaggattttctgtatttgaaagtacctggtctttagtgagagcttcagtgacagatggaagtggtcactttcccaaattaagccctttttactgtatcattgggtgtctgtatatatgacatttaatgctgtgcgcgaccatgtaaaataagcccttctttttctttccttttattttgggctgtaaaaaaaaatgttccgaacaaatatattcagcaaagactgatcattttcaataaaaactccttttttgggaaaaa

SEQ ID NO: 148 - protein, 425 aa, Danio rerio
MAIKFLEVIKPFCAVLPEIQKPERKIQFREKVLWTAITLFIFLVCCQIPLFGIMSSDSADPFYWMRVILASNRGTLMELGISPIVTSGLIMQLLAGAKIIEVGDTPKDRALFNGAQKLFGMIITIGQAIVYVMTGMYGDPSEMGAGICLLIIIQLFVAGLIVLLLDELLQKGYGLGSGISLFIATNICETIVWKAFSPTTVNTGRGTEFEGAIIALFHLLATRTDKVRALREAFYRQNLPNLMNLIATVFVFAVVIYFQGFRVDLPIKSARYRGQYNTYPIKLFYTSNIPIILQSALVSNLYVISQMLSTQFSGNFLVNLLGTWSDTSSGGPARAYPVGGLCYYLSPPESFGSVLDDPVHAVIYIVFMLGSCAFFSKTWIEVSGSSAKDVAKQLKEQHMVMRGHTGRHRLRNGNPIGCYHHLSVL

SEQ ID NO: 149 - DNA, 1820 bp, Danio rerio
cgtttcctttgttgtgatatcgaaaaaaaacgtagtcttacttaaaatctcagcttctggattaagaaatcaagcagttgtcgtctgtcggtattgatcagccgtaaaaatgagtggcggagtctacggaggagatgaagttggagctcttgtctttgatatgggctcgtattcagtgagagctggatatgcaggagaggattgtgccaaggctgattttcctactgtgattggtgtgactctggaccgggaagatggcagtacaccgatggagacagatggggagaaaggaaagcaaagcggaaccacctacttcattgacaccaaccagctcagggtgcctagagaaagcatggaggtcatgtcacctctcaaaaatgggatgattgaggactgggacagttttcaagccattttagatcatacctacaaaatgcacttcaagtcacagcccggtctgcatccagtcctgatgtcagaggcctcgtggaacacacgagcgaaaagagagaagctgacggagctgatgtttgagcattacaacattcccgctttcttcttgtgtaaatcagctgtgctgtcagcgtttgccaatggacgatccacaggcttagtgttagatagcggagcaacacatactactgctattccagtgcatgatggttatgtcctacaacaaggcatcgtaaagtctcctcttgctggtgacttcatgagtatgcaatgtagagagctgtttcaagagttaggtgttgaaatagtgcctccttatatgattgcatcaaaggattcagttcgtgaaggcactcctgctagttggaagaaaaaggagaaactacctcaagttacccgatcatggcataactatatgtgtaataccgtcatccaggattttcaggcctctgtgctccaggtgtcagattcaccctatgatgaacaagtggctgcccagatgcccactgttcactatgagctgcctaatggctacaactgtgactttggagcggaaagacttaagattcctgagggtctttttgacccttcaaatgctaagggtctatcagggaacaccatgttaggagtcggccatgttgtgaccaccagtgttggcatgtgtgacattgacattcgaccgggtctgtatggcagtgtggtagttacaggaggaaataccctcattcaaggctttacagacagacttaacagagaactctcgcagaagacaccacctagcatgcgtcttaagctaatagccaataacacaactgtggagcgccgatttagcgcctggattggaggatccatccttgcatccctgggaactttccaacaaatgtggatttcaaaacaggagtatgaagaaggtggcaaacagtgtgttgacaggaagtgcccttaacacgttccttcaacagctggttgccttcagaaaatgaaatcaaaataaataacacctccacctagtagaaaaagccttgttagtggactcaggaactaaggacacttgctgttatttttagtgcaaaccaacccatattttaaacagtttaacatggttgcactcttttctactctacacaatattttcatatccagtttggcaacacaaacttttccgaccacctaattcatttcagagtaacagtgacaaggttatggggatatgcgaaacctctataatatgtttaggacaattactgtttttttttttttaatttacaccactttgtcactttctagaaaatggatcacagaaaacaaatgtttgttatgcttttaacaactggatcaactgattgaggaataaaattgtttttgtt

SEQ ID NO: 150 - protein, 429 aa, Danio rerio
MSGGVYGGDEVGALVFDMGSYSVRAGYAGEDCAKADFPTVIGVTLDREDGSTPMETDGEKGKQSGTTYFIDTNQLRVPRESMEVMSPLKNGMIEDWDSFQAILDHTYKMHFKSQPGLHPVLMSEASWNTRAKREKLTELMFEHYNIPAFFLCKSAVLSAFANGRSTGLVLDSGATHTTAIPVHDGYVLQQGIVKSPLAGDFMSMQCRELFQELGVEIVPPYMIASKDSVREGTPASWKKKEKLPQVTRSWHNYMCNTVIQDFQASVLQVSDSPYDEQVAAQMPTVHYELPNGYNCDFGAERLKIPEGLFDPSNAKGLSGNTMLGVGHVVTTSVGMCDIDIRPGLYGSVVVTGGNTLIQGFTDRLNRELSQKTPPSMRLKLIANNTTVERRFSAWIGGSILASLGTFQQMWISKQEYEEGGKQCVDRKCP

SEQ ID NO: 151 - DNA, 2213 bp, Danio rerio
gtgcgcagaaagcccggcaggcgcaggctgtaattggagggtaaaatggcgctgagttctcaaggaacaaagaagaaagtttgctattattatgacggggatgtcgggaattactactatggtcagggccatcccatgaagccccacagaatccgcatgacccacaaccttctgctcaactatgggctgtataggaaaatggagatttatcgacctcataaagccaatgcggaagaaatgaccaagtaccacagtgacgactacatcaagttcctccgctccatccgcccagacaacatgtccgagtacagcaagcagatgcagagatttaacgtaggggaggattgtcctgtctttgacggcttatttgagttctgtcagctctcaacaggtggatctgttgctggtgctgtgaaactcaacaaacagcagacagacattgccattaactgggcaggaggtctacatcatgctaagaaatcagaggcatctggattctgctatgtcaacgacattgtactggccattctggagctgctcaagtaccatcaaagagtgctttacatcgacattgacattcatcacggtgatggagttgaggaagccttttacacaacagaccgtgtcatgactgtgtctttccacaagtatggagagtatttcccgggtactggagacctcagagatattggtgcaggcaaggggaagtattacgctgtaaattacccgctcagggatggtattgatgatgagtcctatgaagccatattcaaacctatcatgtccaaagtgatggagatgtaccagcccagtgctgtggtgctccagtgcggcgctgactctctgtccggagatcgattgggctgctttaaccttaccattaaaggtcatgccaagtgtgtagagtacatgaagagctttaacctgccgctgctcatgctgggaggtggaggctacaccattaaaaacgtggcccgctgctggactttcgagacagctgtagcactggacagcaccattcctaatgagctcccgtacaatgattactttgagtattttggacctgacttcaaacttcacatcagccccttcaacatgaccaatcaaaacacaaatgactacctggaaaagatcaagcagcgtctgtttgagaatctgcggatgttgcctcatgctccaggcgttcagatgcaggccatcccggaggatgcagttcaggaagacagcggagatgaagaggacgaccctgacaaacgcatctccattcgtgctcacgataagcggatagcctgtgatgaagagttctcagactctgaggatgaaggccagggtggacggagaaacgcagccaattacaagaagccaaaacgagtgaagactgaagaagagaaggatggagaagagaagaaagatgttaaagaagaggagaaagcttcagaagagaaaatggacacaaaggggccaaaagaagaattaaaaacagtgtgaggtgggggagaaggactgccagtcctcctggatgcatgtacttaagtcgtcaaacaccctcatcttcaaacatcaaacccacatttacacacacacacatacacatacatgctagatgttagctctagtcctgagctaaggactgtaaattattttgtaagactcgttttactatttgtaatctaatgaagcggtataaaagtttttacatttacaagtgtgaaatgaccagatgtttttttgtttcgtgctttggaaccatgtttgtttgcctcccttatgttactatttagcgacgatagcacctcagacacaggaccacaaatagcacataactttactgtcccatcatgggatgcttttttttctcttccccctccccctcaaataattttaaatcatggatggagatacttattggccgctcctacttgggttttctctgcggatgctgtttggtgtccggtgtcctttacctcgtttttaagcgctaaatgtttggtttttattctggtgtctgtctctcgatacctacatctgtgcggtgtgtaagtactccggtatggtcggggcatggcgaaggcttgctgtattgctgttagattcaagatttttctttctgttgtgctttattctagcatgtcagtagtgcccctgaccagacatttagccgtttgattattccagagttcagtggttgtgaaatgtccttattataacaatga

SEQ ID NO: 152 - protein, 480 aa, Danio rerio
MALSSQGTKKKVCYYYDGDVGNYYYGQGHPMKPHRIRMTHNLLLNYGLYRKMEIYRPHKANAEEMTKYHSDDYIKFLRSIRPDNMSEYSKQMQRFNVGEDCPVFDGLFEFCQLSTGGSVAGAVKLNKQQTDIAINWAGGLHHAKKSEASGFCYVNDIVLAILELLKYHQRVLYIDIDIHHGDGVEEAFYTTDRVMTVSFHKYGEYFPGTGDLRDIGAGKGKYYAVNYPLRDGIDDESYEAIFKPIMSKVMEMYQPSAVVLQCGADSLSGDRLGCFNLTIKGHAKCVEYMKSFNLPLLMLGGGGYTIKNVARCWTFETAVALDSTIPNELPYNDYFEYFGPDFKLHISPFNMTNQNTNDYLEKIKQRLFENLRMLPHAPGVQMQAIPEDAVQEDSGDEEDDPDKRISIRAHDKRIACDEEFSDSEDEGQGGRRNAANYKKPKRVKTEEEKDGEEKKDVKEEEKASEEKMDTKGPKEELKTV

SEQ ID NO: 153 - DNA, 1243 bp, Danio rerio
gggattgacctcttcaagcgacacctctccaagaacatccaacaatgagtgagaccgccatctccttcgccaaggacttcttggccggtggtattgccgctgccatctctaaaaccgccgtggcccccattgagagagtcaaactgctgcttcaggtgcaacatgctagcaaacagattacagcagataagcagtacaagggcattatggactgcgtggtgcgtatccccaaggagcagggcttcctgtcgttctggagaggaaacttggccaacgtcatcagatacttccccacacaggccctcaactttgctttcaaggacaagtacaagaaggtcttccttgatggtgtggacaagcgcacccagttttggaggtacttcgctggtaacctggcttcaggtggtgctgctggtgccacatccctctgcttcgtgtatccccttgacttcgcaagaacccgtcttgctgccgatgtcggaaaagctggagcagaaagagagttcagtgggctgggtaactgcttggtaaagatctccaaatctgatggcatcaagggtctgtaccagggcttcaacgtgtccgtgcagggtatcatcatttacagagctgcctacttcggcatttatgacacagccaagggtatgctgcccgatcccaagaacacccatattgttgtgagttggatgattgctcagagtgtgactgctgttgctggtcttgcttcctaccccttcgacacagtgcgtcgtcgtatgatgatgcagtctggacgtaaaggagctgacatcatgtacagtggcacaattgactgctggaggaagatcgcacgtgatgagggtggcaaggctttcttcaagggagcctggtccaacgttctcagaggcatgggtggcgcctttgtgctggtcttgtatgatgagctgaagaaggtcatttaaatgttttcctgtccaccattcaatgtgagatgtttaactgtaacatatcttgtacatttggaaggactctcaaattccgccttatttatagggaccaactagtatgtcagtactagttgtactgggggaaatttgttttgcggtttttcttttgccccgttcatctctcctttttgttcccctcaccgtactgtcattcccagaaagaaagaacaaaagaatgccaggtactaaaatctacctgttttccagccggtctgtttttagtcttcatcaataaagacccactgaatgaacctaaa

SEQ ID NO: 154 - protein, 298 aa, Danio rerio
MSETAISFAKDFLAGGIAAAISKTAVAPIERVKLLLQVQHASKQITADKQYKGIMDCVVRIPKEQGFLSFWRGNLANVIRYFPTQALNFAFKDKYKKVFLDGVDKRTQFWRYFAGNLASGGAAGATSLCFVYPLDFARTRLAADVGKAGAEREFSGLGNCLVKISKSDGIKGLYQGFNVSVQGIIIYRAAYFGIYDTAKGMLPDPKNTHIVVSWMIAQSVTAVAGLASYPFDTVRRRMMMQSGRKGADIMYSGTIDCWRKIARDEGGKAFFKGAWSNVLRGMGGAFVLVLYDELKKVI

SEQ ID NO: 155 - DNA, 1416 bp, Danio rerio
gtgcagcgctaaaatacgccgagcttccgcgagacacttcatcgaggcacagctatgtttatgtgctcaatttagctgttctctgataacaatcagtattatttactaactaagagccaaagatgacctcaaaaacaaaagtgggaaaggtgggcactaaaagcaaagaagatgctcctcatgaactggagagtcagtttattttgcggcttcctcaggaatacgcctctacggttagacggattgcccaatctagcagcatgaacatgaaagacaggcttacaatagagttgcatgctgatggacgccacgggattgtacgcgtggatcgtgttcctttagcatgtaaattggtggatttaccctgcatcctggagtcattaaaaactgttgacaaaaagactttttataagacggccgatatctgtcagatgctggtatgcacactagatggagatctgtacccccctctagaggagcctacaggcactactgactccaaaagcaaaaagaaagacaaggacaaagacaagaaatttgtttggaaccacggcattacacttcctctgaagaacacaagaaagaggcggttcaggaagacagcgaagaagaagtacatcgagtctcctgatgtggaaaaagaggtgaagagactcttgagcacagatgcagaagccgtcagcgttcgatgggaggtgatagctgaagatgaatctaaagaacctgacaacagcttatctctttccaacctggagtcttcacctggaacctctggacacaagggtcatggctcttcagtccaacatgatgagcttcgcgagatcttcaacgacatcagcagcagcagtgaggatgaggacgaagagggcgatcgacatgatgatgaagatctaaacatcatggacactgaagatgacatggtcaggcagctccacgagaaactgaacgagtcagacggaggcagagatgagaatgaccggaacagtcagatcgtaatggagtaccagatgcagatcaacaatttgaaggccaagctccaggaaacacgtgcccgcaagaagcaacaggagaaactgatcatggaggtggaaaatcaagccctgagggatcgcttccagggacttttgaatggtatgattcgtcaagaggagcaagagatggaacagctggcttcccttcaagagcagctggactcgctgatcgagaagtgatgctcaccattaccaagattcagcaatacaagcacactgaccagaggcgactgctgaatacatacacacatgtctgacttcttgtattagctgaatgtgagttgtctaaatatgtgactgaccatctggcctcatcatactgtggattggaaggatcaatcctgcattcgttctttcttttgaccttgtttttagttacttgttt

SEQ ID NO: 156 - protein, 362 aa, Danio rerio
MTSKTKVGKVGTKSKEDAPHELESQFILRLPQEYASTVRRIAQSSSMNMKDRLTIELHADGRHGIVRVDRVPLACKLVDLPCILESLKTVDKKTFYKTADICQMLVCTLDGDLYPPLEEPTGTTDSKSKKKDKDKDKKFVWNHGITLPLKNTRKRRFRKTAKKKYIESPDVEKEVKRLLSTDAEAVSVRWEVIAEDESKEPDNSLSLSNLESSPGTSGHKGHGSSVQHDELREIFNDISSSSEDEDEEGDRHDDEDLNIMDTEDDMVRQLHEKLNESDGGRDENDRNSQIVMEYQMQINNLKAKLQETRARKKQQEKLIMEVENQALRDRFQGLLNGMIRQEEQEMEQLASLQEQLDSLIEK

SEQ ID NO: 157 - DNA, 3050 bp, Danio rerio
tttaaatgtagtgagggaggaaaagacctctgctccctttacgttagagctgatagtctcttatctgagcatccgagaaatactcccgttaatcgcacatcattgccgacgtcctaatgatagatagaggaaaacgattcaagctgtgagactatcaggaaaaaggaaacatactaggacattctgtggtagaagttctgaattagagacaaaaaattgtatttttttatttttatacaaacaggccagtttaaaacgattaaaaatgactagaagatcttgtactatttacgccacagggattgtgtgcgctcatttgctaattttgggaatcgcccttctgctggctcaagtcttccaaacaatgatccaagaacggataaaaaaggagatcacgctggctgagaacagcagagtattggatggatggataaatccaccacctcctgtttacatgcagtatttcttcttcaatgttaccaatcccgatgagttcttggcggggaaggaaaaggccaaagttactcagatggggccatatacctacagggaatatcgaccaagagaaaatgtgacttatcttgagaatgggacaaagatttttgccacaaatcccaagagctttgtgtttcttcgcaacatgtcagctggagatcctgaggttgaccgtgtgacaaccgttaatattccaatgattgcagtgatgaatgagttaaactcctactccttctttgtgaggacagctgtatctatgtatatggggtccatgggcatgggcctgtttatgaatcgtacagttcatgagatcctgtggggtttcaaagatccacttctgaccaaactccacgccatgagaccagaagtggatgagcattttggcctcatgtacaataaaaatggaacccatgaaggtgaatttgtcttccacactggagagaagaattacatgaactatggcaagattgacacgtggaacggcatcagtcagatgaattggtggtcatctaaccagagcaacatgatcaatggtactgatggcagtgtcttccataccttcctgtccagaaaagagctcctctacatctttgctgctgatctctgccgatctatccatttgggttacgtgagggacatggaagtgaagggcatcccagccttccgcttcgctcctccttcagatgtactagctcctccagatgagaacccggcaaatgctggcttctgcgtgcctgctggagactgtctaggaaaaggagtcctcaaagtcagcgtctgtcgtcaaggtgcacccattgttgtgtcattccctcatttctaccaagctgatgagaggtacatcaatgccattgaagggatgaatccaaatgaggaggaacatgaaacatacctcgacatcaacccgaccacaggggttcctattcgagcttgtaagagagctcaactcaatatcatactgaaaagagtgcgtggcttccctaacacaaaatttctcaatgagactattttccccattatgtacgtcaatgagacagcaactattgatgatgagtctgcagcccagatgaggatgctcctgttaattgtaacagtggtatccaactttccagtcatcattttggcattgggggtgattttacttgtagtccttatattcctcgtctgcagaaaccgccagagaaagaatgaagtaaaacgtattgattttactgaagcttttcattcttttgctacaacaaaagatgaaactgcttatacccaagtgagcaaccaagcagaggattcaccagagaaccgcaacaatcagccattgaggaatggatcatatattgccatgtcaccagtggaggcacaaaagtgttgatgaagatgaccgatgaactttcgcctgaggacattatgtctattctcaccattttttaatgtgttttacttttcatcgcagcagtcttctttgggggccagtcaccacctttggctgagggtcacgcattttactaaagacttttttcttttgtgtttgtttgtttccttgagcatggccttctcaaactgccatatcaaagcacccaattcagttttgtagaaccaactctgggtttccagaggcttccgttgcctattcctcagaggcctgaaacacactcttgcctttttgaaaacacaaatatgcattgcatgttcacagagtcgtacaccctctgagcatgagtgttctggatgggtggaaatactggggcccgctgtgctttttaaagtgtttcaaatctcttctcacactactgtatactggtcagcacaggcagaccttgaactaacacatctcttcatatgtcaaaccctttgacaatgagcagacaaatgcaaactgttgatttctcacctctagagtattagggagctaaaaggtctaatatgcttacaaaaactgggtcttatccaggacttttgtgtgtgtgtgtgtgtgtgtgtgtgtgaaagagagagattgcacatatgagtgtgtttgtaattatatgtgagtatttcagcaagcatgtcggtgcttgcatgcttttgtcacattgtggtgaactgctctgttggctcactgtttaaacgacacagtgtgacagcaagtggaaaaaaatagtgacagtcctttccatttcacagtgtctgaagtacattcattgggatgtcagaaacatttcagctgttacaaaaggtcagaaagtcattgtattgcataaatcgcttagtattttataatgaacaaacaacactcacaagtgagacttgaataattctgccttacaaatacttaagaacataatgcggttctgctgcatggcattggcaggtggaactagatgaattgccaacgaaacacgtctgttctgtgatagtgagagcacaaatatagtatgcagatcccatggtctaatcgtagcagaaggggttaaaggtcagcgagttttgagcagtaaagttttgcacaattcttttctgtgcaagtccaatttataataaccctctgtgggtgaaagacctagcacttgaatcactacttg

SEQ ID NO: 158 - protein, 531 aa, Danio rerio
MTRRSCTIYATGIVCAHLLILGIALLLAQVFQTMIQERIKKEITLAENSRVLDGWINPPPPVYMQYFFFNVTNPDEFLAGKEKAKVTQMGPYTYREYRPRENVTYLENGTKIFATNPKSFVFLRNMSAGDPEVDRVTTVNIPMIAVMNELNSYSFFVRTAVSMYMGSMGMGLFMNRTVHEILWGFKDPLLTKLHAMRPEVDEHFGLMYNKNGTHEGEFVFHTGEKNYMNYGKIDTWNGISQMNWWSSNQSNMINGTDGSVFHTFLSRKELLYIFAADLCRSIHLGYVRDMEVKGIPAFRFAPPSDVLAPPDENPANAGFCVPAGDCLGKGVLKVSVCRQGAPIVVSFPHFYQADERYINAIEGMNPNEEEHETYLDINPTTGVPIRACKRAQLNIILKRVRGFPNTKFLNETIFPIMYVNETATIDDESAAQMRMLLLIVTVVSNFPVIILALGVILLVVLIFLVCRNRQRKNEVKRIDFTEAFHSFATTKDETAYTQVSNQAEDSPENRNNQPLRNGSYIAMSPVEAQKC

SEQ ID NO: 159 - protein, 418 aa, Danio rerio
MMRMSWMVTVINRRMMKILIALALIAYIASVWGTYANMRSIQEHGEMKIEQRIDEAVGPLREKIRELELSFTQKYPPVKFLSEKDRKRILITGGAGFVGSHLTDKLMMDGHEVTVVDNFFTGRKRNVEHWIGHENFELINHDVVEPLYIEVDQIYHLASPASPPNYMYNPIKTLKTNTIGTLNMLGLAKRVGARLLLASTSEVYGDPEVHPQNEDYWGHVNPIGPRACYDEGKRVAETMCYAYMKQEGVEVRVARIFNTFGSRMHMNDGRVVSNFILQALQGEALTVYGSGSQTRAFQYVSDLVNGLVSLMNSNISSPVNLGNPEEHTILEFGSLIKSLVASRSHIQFLSEAQDDPQRRRTDIRRAKLLLGWEPVVPLEEGLNKTIQYFSRELEHQANNQYIPKPKAARMKKGRPRHN

(19) United States
(12) Patent Application Publication
(10) Pub. No.: US 2004/0068763 A1
(43) Pub. Date: Apr. 8, 2004

(54) Developmental mutations in zebrafish

(76) Inventors: Nancy Hopkins, Cambridge, MA (US); Gregory Golling, Beaverton, OR (US); Adam Amsterdam, Cambridge, MA (US); Zhoaxia Sun, Quincy, MA (US)

Correspondence Address: CLARK & ELBING LLP, 101 FEDERAL STREET, BOSTON, MA, 02110, US

(21) Appl. No.: 10/403,571

(22) Filed: Mar. 28, 2003

Related U.S. Application Data
(60) Provisional application No. 60/368,760, filed on Mar. 29, 2002.

(51) Int. Cl. A01K 67/027

(52) U.S. Cl. 800/20

(57) ABSTRACT

The invention features novel zebrafish nucleic acid and amino acid molecules, and zebrafish containing mutations in important developmental genes. In addition, the invention features the use of these nucleic acid and amino acid molecules in methods of diagnosing, preventing, and treating a variety of mammalian diseases and developmental disorders. Furthermore, zebrafish mutant for a nucleic acid or amino acid molecule of the invention may be used in screens for compounds that modulate the development of an organism as a whole or of specific tissues or organs within an organism. In particular, the present invention features novel nucleic acid sequences involved, e.g., in kidney development and kidney disorders. Mutations in these sequences, for example, result in the formation of cysts in the kidney.

### CROSS REFERENCE TO RELATED APPLICATIONS

This application claims the benefit of U.S. Provisional Application Serial No. 60/368,760, filed Mar. 29, 2002, the disclosure of which is hereby incorporated by reference.

### FIELD OF THE INVENTION

The field of the invention is developmental diseases and disorders.

### BACKGROUND OF THE INVENTION

Genetic screens have been the most successful approach for identifying genes required for developmental processes. Applied on a sufficiently large scale, a genetic screen can identify all of the genes which, when mutated one at a time, impact the phenotype of interest. Notably, genetic screens are relatively unbiased since no assumptions about the genes involved in the biological processes of interest need to be made and, thus, such screens can reveal novel genetic pathways underlying important phenotypes.

It has long been recognized in the art that genetic screens in vertebrate animals would be highly informative and would help identify many new genes required for the development of vertebrate organs and structures and such genetic screens have been carried out in mice and zebrafish (Rinchik, Trends Genet. 7:15-21, 1991; Driever et al., Development 123:37-46, 1996; Haffter et al., Development 123:1-36, 1996). Genetic screens using chemical mutagens in zebrafish suggested that there are only about 2,400 genes that can be mutated to yield a visible phenotype affecting the development of a fertilized egg to a free-swimming five-day old larva (Haffter et al., Development 123:1-36, 1996). In accordance to what was observed in invertebrates, these results suggest that a small number of genes may be essential for the development of a viable vertebrate (Driever et al., Development 123:37-46, 1996; Haffter et al., Development 123:1-36, 1996; Dove, Genetics 116:5-8, 1987). In other respects, however, genetic screens in vertebrates have not been nearly as informative as those in invertebrates. The difficulty of performing large enough screens to identify all the genes required for a specific biological process, and the difficulty of rapidly cloning mutated genes from vertebrate genomes contribute to this shortcoming. Nevertheless, the small-scale screens that have been performed in zebrafish and mice hint at the vast potential of this approach.

Simply identifying mutant phenotypes in a genetic screen can be informative by revealing both the kinds of phenotypes that can occur and the number of genes involved in the process of interest. In zebrafish, simple visual screens of embryos in the first 5 days after fertilization can reveal mutations in genes essential for the normal development of most of the major organ systems, including the nervous system, heart, blood, gut, liver, kidney, jaws, eyes, and ears. However, to understand how genes specify a biological process, it is essential to identify the mutated genes responsible for the phenotypes.

Insertional mutagenesis, when compared to chemical mutagenesis, greatly speeds cloning the mutated gene. The integration of exogenous DNA sequences into a genome can be mutagenic, and simplifies cloning of the mutated genes since the inserted DNA serves as a tag to aid in isolating the flanking DNA sequence. Previously, insertional mutagens, including DNA viruses as well as retroviruses, have been used successfully in Drosophila and mice. For example, mouse retroviral vectors pseudotyped with a VSV-G envelope were found to be able to infect the fish germ line following injection of virus into blastula-stage embryos at the 1000 to 2000-cell stage. In addition, retroviruses were attractive candidates for insertional mutagens, because they had been shown to integrate into many different sites in mammalian and avian chromosomes and to be effective mutagens in mice. Importantly, they integrate without rearrangement of their own sequences or significant alterations to host DNA sequences at the site of insertion, essential features for easily cloning genes disrupted by insertions. Applying this approach to zebrafish to identify and clone genes important in zebrafish development is desirable and is likely to provide significant insights into many aspects of vertebrate development and, thereby, aid in our understanding, diagnosis, and treatment of a variety of diseases, including ones that affect humans.

### SUMMARY OF THE INVENTION

The invention features novel zebrafish nucleic acid and amino acid molecules, and zebrafish containing mutations in important developmental genes. In addition, the invention features the use of these nucleic acid and amino acid molecules in methods of diagnosing, preventing, and treating a variety of mammalian diseases and developmental disorders. Furthermore, zebrafish mutant for a nucleic acid or amino acid molecule of the invention may be used in screens for compounds that modulate the development of an organism as a whole or of specific tissues or organs within an organism. In particular, the present invention features novel nucleic acid sequences involved, e.g., in kidney development and kidney disorders. Mutations in these sequences, for example, result in the formation of cysts in the kidney.

Accordingly, the first aspect of the invention features an isolated nucleic acid molecule, e.g., a mouse, human, or zebrafish nucleic acid moleucle, including a nucleic acid sequence having at least 75% nucleic acid sequence identity to the 459 nucleic acid sequence of SEQ ID NO:59 over at least 600 contiguous nucleic acids, where this nucleic acid molecule functions in kidney development. In desirable embodiments of this aspect, the nucleic acid sequence includes the sequence of SEQ ID NO:59. In addition, the nucleic acid molecule may include a viral insertion at a nucleotide corresponding to nucleotide 210 of SEQ ID NO:59. Desirably, the invention features a zebrafish containing a viral insertion at a nucleotide corresponding to nucleotide 210 of SEQ ID NO:59 Further desirable embodiments are vector including the isolated nucleic acid molecule of this aspect of the invention and a cell including this vector.

In another aspect, the invention features an isolated polypeptide including an amino acid sequence having at least 75% sequence identity to the 459 amino acid sequence of SEQ ID NO:60 over at least 250 contiguous amino acids, where this polypeptide functions in kidney development. Desirably, the polypeptide includes the sequence of SEQ ID NO:60.

The invention also features a method of treating or preventing a kidney disorder in an organism. This method includes the step of contacting the organism with a therapeutically effective amount of the nucleic acid of the first aspect of the invention, or its complement, where the nucleic acid is sufficient to elicit an alteration in expression of a 459 nucleic acid sequence in the organism, and where the alteration in the level of expression treats or prevents a kidney disorder. The nucleic acidmolecule used in this method can be a cDNA or an mRNA molecule and the contacting can result in an increase in expression of the polypeptide encoded by a nucleic acid sequence including the sequence of SEQ ID NO:59. Alternatively, the nucleic acid molecule used in this method can be a double-stranded RNA molecule and the contacting can lead to a decrease in expression, or inhibits biological activity, of a nucleic acid sequence including SEQ ID NO:59. Further, the nucleic acid molecule used in this method may be an anti-sense RNA molecule, and the contacting can lead to a decrease in expression, or inhibition of biological activity, of a nucleic acid sequence including the sequence of SEQ ID NO:59.

The invention also features method for diagnosing a kidney disorder or the propensity to develop a kidney disorder in an organism. This method includes detecting an alteration in the level of 459 polypeptide expression in a sample derived from a first organism and comparing the level of expression to that of a 459 polypeptide in a sample derived from a second, control organism, where an alteration in the level of expression or activity of the 459 polypeptide in the first organism relative to the second organism is indicative of the first organism having or having a propensity to develop a kidney disorder. Desirably, the 459 polypeptide includes the amino acid sequence of SEQ ID NO:60.

Further, the invention features another method for diagnosing a kidney disorder or the propensity to develop a kidney disorder in an organism. This method involves detecting an alteration in the sequence, or a fragment of the sequence, of a 459 nucleic acid molecule in a sample derived from a first organism and comparing the sequence to that of a 459 nucleic acid molecule derived from a second, control organism, where an alteration of the 459 sequence in the first organism relative to the second organism is indicative of the first organism having or having a propensity to develop a kidney disorder. In desirable embodiments of this aspect of the invention, the alteration is a decrease in the expression or activity of a nucleic acid molecule including the sequence of SEQ ID NO:59 or, alternatively, the alteration is an increase in the expression or activity of a nucleic acid molecule including the sequence of SEQ ID NO:59.

Another aspect, the invention features an isolated nucleic acid molecule including a sequence having at least 85% nucleic acid sequence identity to the 904 nucleic acid sequence of SEQ ID NO:1 over at least 500 contiguous nucleic acids, where this nucleic acid molecule functions in neural tissue proliferation, Central Nervous System (CNS) development, or vascular development. In addition, this isolated nucleic acid sequence may include a mutation, for example, a viral insertion, such as, one at a nucleotide corresponding to nucleotide 1315 of SEQ ID NO:1.

In a further aspect, the invention features an isolated nucleic acid molecule including a sequence having at least 85% nucleic acid sequence identity to the U2AF nucleic acid sequence of SEQ ID NO:7 over at least 600 contiguous nucleic acids, where this nucleic acid molecule functions in brain or tectum development. In addition, this isolated nucleic acid molecule may include a mutation, for example, a viral insertion, such as, one between nucleotides corresponding to nucleotides 46 and 47 of SEQ ID NO:7.

In a further aspect, the invention features an isolated nucleic acid molecule including a sequence having at least 40% nucleic acid sequence identity to the 954 nucleic acid sequence of SEQ ID NO:9 over at least 500 contiguous nucleic acids, where this nucleic acid molecule functions in cartilage development. In addition, this isolated nucleic acid molecule may include a mutation, for example, a viral insertion, such as, one at a nucleotide corresponding to nucleotide 432 or 506 of SEQ ID NO:9.

In a further aspect, the invention features an isolated nucleic acid molecule including a sequence having at least 90% nucleic acid sequence identity to the V-ATPase Alpha Subunit nucleic acid sequence of SEQ ID NO:15 over at least 250 contiguous nucleic acids, where this nucleic acid molecule functions in eye or body pigmentation. In addition, this isolated nucleic acid molecule may include a mutation, for example, a viral insertion, such as, one at a nucleotide corresponding to nucleotide 169 of SEQ ID NO:15.

In a further aspect, the invention features an isolated nucleic acid molecule including a sequence having at least 85% nucleic acid sequence identity to the V-ATPase SFD Subunit nucleic acid sequence of SEQ ID NO:17 over at least 600 contiguous nucleic acids, where this nucleic acid molecule functions in eye or body pigmentation. In addition, this isolated nucleic acid molecule may include a mutation, for example, a viral insertion, such as, one between nucleotides corresponding to nucleotides 31 and 32 of SEQ ID NO:17.

In a further aspect, the invention features an isolated nucleic acid molecule including a sequence having at least 90% nucleic acid sequence identity to the V-ATPase-16 kDa Proteolytic Subunit nucleic acid sequence of SEQ ID NO:19 over at least 400 contiguous nucleic acids, where this nucleic acid molecule functions in body or eye pigmentation or touch sensitivity. In addition, this isolated nucleic acid molecule may include a mutation, for example, a viral insertion, such as, one between nucleotides corresponding to nucleotides 242 and 243 of SEQ ID NO:19.

In a further aspect, the invention features an isolated nucleic acid molecule including a sequence having at least 75% nucleic acid sequence identity to the 1463 nucleic acid sequence of SEQ ID NO:157 over at least 200 contiguous nucleic acids, where this nucleic acid molecule functions in body pigmentation, brain development, or vascular development. In addition, this isolated nucleic acid molecule may include a mutation, for example, a viral insertion, such as, one between nucleotides corresponding to nucleotides 389 and 390 of SEQ ID NO:157.

In an further aspect, the invention features an isolated nucleic acid molecule including a sequence having at least 90% nucleic acid sequence identity to the VPSP18 nucleic acid sequence of SEQ ID NO:21 over at least 75 contiguous nucleic acids, where this nucleic acid molecule functions in pigmentation, photoreceptor, retinal, or brain development. In addition, this isolated nucleic acid molecule may include a mutation, for example, a viral insertion, such as, one at a nucleotide corresponding to nucleotide 2336 of SEQ ID NO:21.

In a further aspect, the invention features an isolated nucleic acid molecule including a sequence having at least 85% nucleic acid sequence identity to the 60S Ribosomal Protein L35 nucleic acid sequence of SEQ ID NO:25 over at least 350 contiguous nucleic acids, where this nucleic acid molecule functions in head, eye, or brain development. In addition, this isolated nucleic acid molecule may include a mutation, for example, a viral insertion, such as, one between nucleotides corresponding to nucleotides 30 and 31 of SEQ ID NO:25.

In a further aspect, the invention features an isolated nucleic acid molecule including a sequence having at least 85% nucleic acid sequence identity to the 215 nucleic acid sequence of SEQ ID NO:31 over at least 200 contiguous nucleic acids, where this nucleic acid molecule functions in eye or jaw development. In addition, this isolated nucleic acid molecule may include a mutation, for example, a viral insertion, such as, one between nucleotides corresponding to nucleotides 294 and 295 of SEQ ID NO:31.

In a further aspect, the invention features an isolated nucleic acid molecule including a nucleic acid sequence having at least 95% nucleic acid sequence identity to the 307 nucleic acid sequence of SEQ ID NO:33 over at least 50 contiguous nucleic acids, where this nucleic acid molecule functions in jaw or cartilage development. In addition, this isolated nucleic acid molecule may include a mutation, for example, a viral insertion, such as, one at a nucleotide corresponding to nucleotide 176 of SEQ ID NO:33.

In a further aspect, the invention features an isolated nucleic acid molecule including a nucleic acid sequence having at least 40% nucleic acid sequence identity to the 572 nucleic acid sequence of SEQ ID NO:35, where this nucleic acid functions in jaw or cartilage development. In addition, this isolated nucleic acid molecule may include a mutation, for example, a viral insertion, such as, one at a nucleotide corresponding to nucleotide 277 of SEQ ID NO:35.

In a further aspect, this invention features an isolated nucleic acid molecule including a nucleic acid sequence having at least 42% nucleic acid sequence identity to the 1116A nucleic acid sequence of SEQ ID NO:37 over at least 1000 contiguous nucleic acids, where this nucleic acid molecule functions in jaw development. In addition, this isolated nucleic acid molecule may include a mutation, for example, a viral insertion, such as, one at a nucleotide corresponding to nucleotide 135 of SEQ ID NO:37.

In a further aspect, the invention features an isolated nucleic acid molecule including a nucleic acid sequence having at least 80% nucleic acid sequence identity over at least 1000 contiguous nucleic acids to the 1548 nucleic acid sequence of SEQ ID NO:39, where this nucleic acid molecule functions in eye, head, heart, limb, jaw, or neurocranium development. In addition, this isolated nucleic acid molecule may include a mutation, for example, a viral insertion, such as, one at a nucleotide corresponding to nucleotide 85 of SEQ ID NO:39.

In a further aspect, the invention features an isolated nucleic acid molecule including a nucleic acid sequence having at least 85% nucleic acid sequence identity to Casein Kinase 1α nucleic acid sequence of SEQ ID NO:41 over at least 1000 contiguous nucleic acids, where this nucleic acid molecule functions in jaw, limb, or cartilage development. In addition, this isolated nucleic acid molecule may include a mutation, for example, a viral insertion, such as, one between nucleotides corresponding to nucleotides 730 and 731 of SEQ ID NO:41.

In a further aspect, the invention features an isolated nucleic acid molecule including a nucleic acid sequence having at least 80% nucleic acid sequence identity to the 429 nucleic acid sequence of SEQ ID NO:47 over at least 200 contiguous nucleic acids, where this nucleic acid molecule functions in vascular, liver, gall bladder, pancreas, or gut development. In addition, this isolated nucleic acid molecule may include a mutation, for example, a viral insertion, such as, one between nucleotides corresponding to nucleotides 182 and 183 of SEQ ID NO:47.

In a further aspect, the invention features an isolated nucleic acid molecule including a nucleic acid sequence having at least 85% nucleic acid sequence identity to the 428 nucleic acid sequence of SEQ ID NO:49 over at least 200 contiguous nucleic acids, where this nucleic acid molecule functions in muscle or brain development. In addition, this isolated nucleic acid molecule may include a mutation, for example, a viral insertion, such as, one at a nucleotide corresponding to nucleotide 187 of SEQ ID NO:49.

In a further aspect, the invention features an isolated nucleic acid molecule including a nucleic acid sequence having at least 85% nucleic acid sequence identity to the Spinster nucleic acid sequence of SEQ ID NO:51 over at least 100 contiguous nucleic acids, where this nucleic acid molecule functions in infertility disorders. In addition, this isolated nucleic acid molecule may include a mutation, for example, a viral insertion, such as, one at a nucleotide corresponding to a nucleotide approximately 2 to 5 kb upstream of nucleotide 209 of SEQ ID NO:51.

In a further aspect, the invention features an isolated nucleic acid molecule including a nucleic acid sequence having at least 85% nucleic acid sequence identity to the Kinesin-Related Motor Protein EGS nucleic acid sequence of SEQ ID NO:57 over at least 600 nucleic acids, where this nucleic acid molecule functions in cell death regulation or body development. In addition, this isolated nucleic acid molecule may include a mutation, for example, a viral insertion, such as, one between nucleotides corresponding to nucleotides 50 and 51 of SEQ ID NO:57.

In a further aspect, the invention features an isolated nucleic acid molecule including a nucleic acid sequence having at least 75% nucleic acid sequence identity to the 459 nucleic acid sequence of SEQ ID NO:59 over at least 600 contiguous nucleic acids, where this nucleic acid molecule functions in cell death regulation in the CNS or in body development. In addition, this isolated nucleic acid molecule may include a mutation, for example, a viral insertion, such as, one at a nucleotide corresponding to nucleotide 210 of SEQ ID NO:59.

In a further aspect, the invention features an isolated nucleic acid molecule including a nucleic acid sequence having at least 80% nucleic acid sequence identity to the Vesicular Integral-Membrane Protein VIP 36 nucleic acid sequence of SEQ ID NO:65 over at least 300 contiguous nucleic acids, where this nucleic acid molecule functions in touch sensitivity. In addition, this isolated nucleic acid molecule may include a mutation, for example, a viral insertion, such as one between nucleotides corresponding to nucleotides 219 and 220 of SEQ ID NO:65.

In a further aspect, the invention features an isolated nucleic acid molecule including a nucleic acid sequence having at least 85% nucleic acid sequence identity to the 299 nucleic acid sequence of SEQ ID NO:67 over at least 100 contiguous nucleic acids, where this nucleic acid molecule functions in brain or eye apoptosis, or in jaw, cartilage, or limb development. In addition, this isolated nucleic acid molecule may include a mutation, for example, a viral insertion, such as, one between nucleotides corresponding to nucleotides 47 and 48 of SEQ ID NO:67.

In a further aspect, the invention features an isolated nucleic acid molecule including a nucleic acid sequence having at least 40% nucleic acid sequence identity to the 994 nucleic acid sequence of SEQ ID NO:69 over at least 500 contiguous nucleic acids, where this nucleic acid molecule functions in eye, head, jaw, cartilage, or stomach development. In addition, this isolated nucleic acid molecule may include a mutation, for example, a viral insertion, such as, one between nucleotides corresponding to nucleotides 66 and 67 of SEQ ID NO:69.

In a further aspect, the invention features an isolated nucleic acid molecule including a nucleic acid sequence having at least 85% nucleic acid sequence identity to the 1373 nucleic acid sequence of SEQ ID NO:71 over at least 350 contiguous nucleic acids, where this nucleic acid molecule functions in brain, eye, or body development. In addition, this isolated nucleic acid molecule may include a mutation, for example, a viral insertion, such as, one between nucleotides corresponding to nucleotides 118 and 119 of SEQ ID NO:71.

In a further aspect, the invention features an isolated nucleic acid molecule including a nucleic acid sequence having at least 80% nucleic acid sequence identity to the Denticleless nucleic acid sequence of SEQ ID NO:73 over at least 200 contiguous nucleic acids, where this nucleic acid molecule functions in CNS, body, somite, yolk sac, or eye development. In addition, this isolated nucleic acid molecule may include a mutation, for example, a viral insertion, such as, one between nucleotides corresponding to nucleotides 307 and 308 of SEQ ID NO:73.

In a further aspect, the invention features an isolated nucleic acid molecule including a nucleic acid sequence having at least 35% nucleic acid sequence identity to the Telomeric Repeat Factor 2 nucleic acid sequence of SEQ ID NO:79 over at least 250 contiguous nucleic acids, where this nucleic acid molecule functions in brain or eye development. In addition, this isolated nucleic acid molecule may include a mutation, for example, a viral insertion, such as, one between nucleotides corresponding to nucleotides 529 and 530 of SEQ ID NO:79.

In a further aspect, the invention features an isolated nucleic acid molecule including a nucleic acid sequence having at least 80% nucleic acid sequence identity to the SIL nucleic acid sequence of SEQ ID NO:81 over at least 100 contiguous nucleic acids, where this nucleic acid molecule functions in brain, head, or body development, or in motility. In addition, this isolated nucleic acid molecule may include a mutation, for example, a viral insertion, such as, one between nucleotides corresponding to nucleotides 273 and 274 of SEQ ID NO:81.

In a further aspect, the invention features an isolated nucleic acid molecule including a nucleic acid sequence having at least 85% nucleic acid sequence identity to the U1 Small Nuclear Ribonucleoprotein C nucleic acid sequence of SEQ ID NO:83 over at least 200 contiguous nucleic acids, where this nucleic acid molecule functions in body, eye, hindbrain, ear, pigment, or limb development. In addition, this isolated nucleic acid molecule may include a mutation, for example, a viral insertion, such as, one between nucleotides corresponding to nucleotides 52 and 53 of SEQ ID NO:83.

In a further aspect, the invention features an isolated nucleic acid molecule including a nucleic acid sequence having at least 80% nucleic acid sequence identity to Ski Interacting Protein nucleic acid sequence of SEQ ID NO:85 over at least 850 contiguous nucleic acids, where this nucleic acid molecule functions in brain or body development, or motility. In addition, this isolated nucleic acid molecule may include a mutation, for example, a viral insertion, such as, one at a nucleotide corresponding to a nucleotide approximately 1.2 kb upstream of nucleotide 19 of SEQ ID NO:85.

In a further aspect, the invention features an isolated nucleic acid molecule including a nucleic acid sequence having at least 85% nucleic acid sequence identity to the 297 nucleic acid sequence of SEQ ID NO:87 over at least 200 contiguous nucleic acids, where this nucleic acid molecule functions in motility, cartilage, jaw, eye, tail, or brain development. In addition, this isolated nucleic acid molecule may include a mutation, for example, a viral insertion, such as, one at a nucleotide corresponding to nucleotide 74 of SEQ ID NO:87.

In a further aspect, the invention features an isolated nucleic acid molecule including a nucleic acid sequence having at least 80% nucleic acid sequence identity to the TCP-1 Complex Gamma Chain nucleic acid sequence of SEQ ID NO:89 over at least 1500 contiguous nucleic acids, where this nucleic acid molecule functions in yolk sac development. In addition, this isolated nucleic acid molecule may include a mutation, for example, a viral insertion, such as, one between nucleotides corresponding to nucleotides 75 and 76 of SEQ ID NO:89.

In a further aspect, the invention features an isolated nucleic acid molecule including a nucleic acid sequence having at least 90% nucleic acid sequence identity to the Small Nuclear Ribonucleoprotein D1 nucleic acid sequence of SEQ ID NO:91 over at least 200 contiguous nucleic acids, where this nucleic acid molecule functions in CNS or eye development. In addition, this isolated nucleic acid molecule may include a mutation, for example, a viral insertion, such as, one between nucleotides corresponding to nucleotides 76 and 77 of SEQ ID NO:91.

In a further aspect, the invention features an isolated nucleic acid molecule including a nucleic acid sequence having at least 80% nucleic acid sequence identity to the DNA Polymerase Epsilon Subunit B nucleic acid sequence of SEQ ID NO:93 over at least 1050 contiguous nucleic acids, where this nucleic acid molecule functions in brain or eye development. In addition, this isolated nucleic acid molecule may include a mutation, for example, a viral insertion, such as, one at a nucleotide corresponding to nucleotide 929 of SEQ ID NO:93 or one between nucleotides corresponding to nucleotides 1161 and 1162 of SEQ ID NO:93.

In a further aspect, the invention features an isolated nucleic acid molecule including a nucleic acid sequence having at least 80% nucleic acid sequence identity to the 821-02 nucleic acid sequence of SEQ ID NO:95 over at least 100 contiguous nucleic acids, where this nucleic acid molecule functions in cell death regulation in the CNS or the eye. In addition, this isolated nucleic acid molecule may include a mutation, for example, a viral insertion, such as, one between nucleotides corresponding to nucleotides 231 and 232 or 369 and 370 of SEQ ID NO:95.

In a further aspect, the invention features an isolated nucleic acid molecule including a nucleic acid sequence having at least 80% nucleic acid sequence identity to the 1045 nucleic acid sequence of SEQ ID NO:97 over at least 600 contiguous nucleic acids, where this nucleic acid molecule functions in brain or head development. In addition, this isolated nucleic acid molecule may include a mutation, for example, a viral insertion, such as, one between nucleotides corresponding to nucleotides 216 and 344 of SEQ ID NO:97.

In a further aspect, the invention features an isolated nucleic acid molecule including a nucleic acid sequence having at least 75% nucleic acid sequence identity to the 1055-1 nucleic acid sequence of SEQ ID NO:99 over at least 600 contiguous nucleic acids, where this nucleic acid molecule functions in cell cycle progression or 60S ribosomal subunit biogenesis. In addition, this isolated nucleic acid molecule may include a mutation, for example, a viral insertion, such as, one between nucleotides corresponding to nucleotides 167 and 168 of SEQ ID NO:99.

In a further aspect, the invention features an isolated nucleic acid molecule including a nucleic acid sequence having at least 80% nucleic acid sequence identity to the Spliceosome Associated Protein 49 nucleic acid sequence of SEQ ID NO:101 over at least 700 contiguous nucleic acids, where this nucleic acid molecule functions in tectal or body development. In addition, this isolated nucleic acid molecule may include a mutation, for example, a viral insertion, such as, one between nucleotides corresponding to nucleotides 53 and 54 of SEQ ID NO:101.

In a further aspect, the invention features an isolated nucleic acid molecule including a nucleic acid sequence having at least 80% nucleic acid sequence identity to the DNA Replication Licensing Factor MCM7 nucleic acid sequence of SEQ ID NO:103 over at least 300 contiguous nucleic acids, where this nucleic acid molecule functions in eye or CNS development. In addition, this isolated nucleic acid molecule may include a mutation, for example, a viral insertion, such as, one at a nucleotide corresponding to nucleotide 198 of SEQ ID NO:103, or one between nucleotides corresponding to nucleotides 121 and 122 of SEQ ID NO:103.

In a further aspect, the invention features an isolated nucleic acid molecule including a nucleic acid sequence having at least 85% nucleic acid sequence identity to the Dead-Box RNA Helicase (DEAD5 or DEAD19) nucleic acid sequence of SEQ ID NO:105 over at least 900 contiguous nucleic acids, where this nucleic acid molecule functions in brain development. In addition, this isolated nucleic acid molecule may include a mutation, for example, a viral insertion, such as, one at a nucleotide corresponding to nucleotide 132 of SEQ ID NO:105.

In a further aspect, the invention features an isolated nucleic acid molecule including a nucleic acid sequence having at least 80% nucleic acid sequence identity to the 1581 nucleic acid sequence of SEQ ID NO:107 over at least 200 contiguous nucleic acids, where this nucleic acid molecule functions in head or eye development. In addition, this isolated nucleic acid molecule may include a mutation, for example, a viral insertion, such as, one between nucleotides corresponding to nucleotides 346 and 347 of SEQ ID NO:107.

In a further aspect, the invention features an isolated nucleic acid molecule including a nucleic acid sequence having at least 90% nucleic acid sequence identity to the Imitation Switch (ISWI)/SNF2 nucleic acid sequence of SEQ ID NO:111 over at least 200 contiguous nucleic acids, where this nucleic acid molecule functions in development of the visual system, brain, jaw or cartilage. In addition, this isolated nucleic acid molecule may include a mutation, for example, a viral insertion, such as, one at a nucleotide corresponding to nucleotide 76 of SEQ ID NO:111.

In a further aspect, this invention features an isolated nucleic acid molecule including a nucleic acid sequence having at least 80% nucleic acid sequence identity to the Chromosomal Assembly Protein C (XCAP-C) nucleic acid sequence of SEQ ID NO:113 over at least 600 contiguous nucleic acids, where this nucleic acid molecule functions in optic tectum, eye, or hindbrain development. In addition, this isolated nucleic acid molecule may include a mutation, for example, a viral insertion, such as, one between nucleotides corresponding to nucleotides 181 and 182 of SEQ ID NO:113.

In a further aspect, the invention features an isolated nucleic acid molecule including a nucleic acid sequence having at least 80% nucleic acid sequence identity to the DNA Replication Licensing Factor MCM2 nucleic acid sequence of SEQ ID NO:115 over at least 1200 contiguous nucleic acids, where this nucleic acid molecule functions in eye, optic tectum, jaw, or cartilage development. In addition, this isolated nucleic acid molecule may include a mutation, for example, a viral insertion, such as, one at a nucleotide in an corresponding to a nucleotide in an intron preceding nucleotide 399 of SEQ ID NO:115.

In a further aspect, the invention features an isolated nucleic acid molecule including a nucleic acid sequence having at least 80% nucleic acid sequence identity to the DNA Replication Licensing Factor MCM3 nucleic acid sequence of SEQ ID NO:117 over at least 600 contiguous nucleic acids, where this nucleic acid molecule functions in optic tectum, head, or eye development. In addition, this isolated nucleic acid molecule may include a mutation, for example, a viral insertion, such as, one at a nucleotide corresponding to nucleotide 50 of SEQ ID NO:117, or one between nucleotides corresponding to nucleotides 75 and 76 of SEQ ID NO:117.

In a further aspect, the invention features an isolated nucleic acid molecule including a Valyl-tRNA Synthase nucleic acid sequence of SEQ ID NO:119, where this nucleic acid molecule functions in cell death regulation, head or eye development, or eye pigmentation. In addition, this isolated nucleic acid molecule may include a mutation, for example, a viral insertion, such as, one between nucleotides corresponding to nucleotides 30 and 31 of SEQ ID NO:119.

In a further aspect, the invention features an isolated nucleic acid molecule including a nucleic acid sequence having at least 85% nucleic acid sequence identity to the 405 Ribosomal Protein S5 nucleic acid sequence of SEQ ID NO:121 over at least 600 contiguous nucleic acids, where this nucleic acid molecule functions in brain development or motility. In addition, thisisolated nucleic acid molecule may include a mutation, for example, a viral insertion, such as, one between nucleotides corresponding to nucleotides 31 and 32 of SEQ ID NO:121.

In a further aspect, the invention features an isolated nucleic acid molecule including a nucleic acid sequence having at least 80% nucleic acid sequence identity to the TCP-1 Beta nucleic acid sequence of SEQ ID NO:123 over at least 1500 contiguous nucleic acids, where this nucleic acid molecule functions in jaw, cartilage, head, or eye development. In addition, this isolated nucleic acid molecule may include a mutation, for example, a viral insertion, such as, one between nucleotides corresponding to nucleotides 63 and 64 of SEQ ID NO:123.

In a further aspect, the invention features an isolated nucleic acid molecule including a nucleic acid sequence having at least 80% nucleic acid sequence identity to the TCP-1 Eta nucleic acid sequence of SEQ ID NO:125 over at least 1600 contiguous nucleic acids, where this nucleic acid molecule functions in head, brain, or eye development. In addition, this isolated nucleic acid molecule may include a mutation, for example, a viral insertion, such as, one between nucleotides corresponding to nucleotides 32 and 33 of SEQ ID NO:125.

In a further aspect, the invention features an isolated nucleic acid molecule including the Translation Elongation Factor eEF1 Alpha nucleic acid sequence of SEQ ID NO:127, where this nucleic acid molecule functions in cell death regulation in the head or eye. In addition, this isolated nucleic acid molecule may include a mutation, for example, a viral insertion, such as, one between nucleotides corresponding to nucleotides 60 and 61 of SEQ ID NO:127.

In a further aspect, this invention features an isolated nucleic acid molecule including a nucleic acid sequence having at least 40% identity to the 1257 nucleic acid sequence of SEQ ID NO:129 over at least 500 contiguous nucleic acids where this nucleic acid molecule functions in head, eye, or jaw development. In addition, this isolated nucleic acid molecule may include a mutation, for example, a viral insertion, such as, one at a nucleotide corresponding to nucleotide 175 of SEQ ID NO:129.

In a further aspect, the invention features an isolated nucleic acid molecule including a nucleic acid sequence having at least 85% nucleic acid sequence identity to the 60S Ribosomal Protein L24 nucleic acid sequence of SEQ ID NO:131 over at least 400 contiguous nucleic acids, where this nucleic acid molecule functions in head or eye development. In addition, this isolated nucleic acid molecule may include a mutation, for example, a viral insertion, such as, one between nucleotides corresponding to nucleotides 144 and 145 of SEQ ID NO:131.

In a further aspect, the invention features an isolated nucleic acid molecule including a nucleic acid sequence having at least 80% nucleic acid sequence identity to the Non-Muscle Adenylosuccinate Synthase nucleic acid sequence of SEQ ID NO:133 over at least 350 contiguous nucleic acids, where this nucleic acid molecule functions in cell death regulation, or in jaw or cartilage development. In addition, this isolated nucleic acid molecule may include a mutation, for example, a viral insertion, such as, one between nucleotides corresponding to nucleotides 217 and 218 of SEQ ID NO:133, or one at a nucleotide corresponding to nucleotide 209 of SEQ ID NO:133.

In a further aspect, the invention features an isolated nucleic acid molecule including a nucleic acid sequence having at least 75% nucleic acid sequence identity to the Nuclear Cap Binding Protein Subunit 2 nucleic acid sequence of SEQ ID NO:135 over at least 450 contiguous nucleic acids, where this nucleic acid molecule functions in head, eye, CNS, jaw, cartilage, or stomach development. In addition, this isolated nucleic acid molecule may include a mutation, for example, a viral insertion, such as, one between nucleotides corresponding to nucleotides 137 and 138 of SEQ ID NO:135.

In a further aspect, the invention features an isolated nucleic acid molecule including the Ornithine Decarboxylase nucleic acid sequence of SEQ ID NO:137, where this nucleic acid molecule functions in jaw or cartilage development. In addition, this isolated nucleic acid molecule may include a mutation, for example, a viral insertion, such as, one between nucleotides corresponding to nucleotides 97 and 98 of SEQ ID NO:137.

In a further aspect, this invention features an isolated nucleic acid molecule including a nucleic acid sequence having at least 80% nucleic acid sequence identity to the full length Protein Phosphatase 1 Nuclear Targeting Subunit nucleic acid sequence of SEQ ID NO:139 over at least 250 contiguous nucleic acids, where this nucleic acid molecule functions in head, jaw, body, or gut development. In addition, this isolated nucleic acid molecule may include a mutation, for example, a viral insertion, such as, one at a nucleotide corresponding to nucleotide 303 of SEQ ID NO:139.

In a further aspect, this invention features an isolated nucleic acid molecule including a nucleic acid sequence having at least 80% nucleic acid sequence identity to the Mitochondrial Inner Membrane Translocating (rTIM23) nucleic acid sequence of SEQ ID NO:141 over at least 450 contiguous nucleic acids, where this nucleic acid molecule functions in eye pigmentation or in the development of the vascular system. In addition, this isolated nucleic acid molecule may include a mutation, for example, a viral insertion, such as, one at a nucleotide corresponding to nucleotide 100 of SEQ ID NO:141.

In a further aspect, the invention features an isolated nucleic acid molecule including a nucleic acid sequence having at least 80% nucleic acid sequence identity to the 1447 nucleic acid sequence of SEQ ID NO:143 over at least 950 contiguous nucleic acids, where this nucleic acid molecule functions in pancreas, tail, stomach, cartilage, or limb development. In addition, this isolated nucleic acid molecule may include a mutation, for example, a viral insertion, such as, one between nucleotides corresponding to nucleotides 227 and 228 of SEQ ID NO:143.

In a further aspect, the invention features an isolated nucleic acid molecule including a nucleic acid sequence having at least 80% nucleic acid sequence identity to the ARS2 nucleic acid sequence of SEQ ID NO:145 over at least 650 contiguous nucleic acids, where this nucleic acid molecule functions in pigment, tectum, jaw, or ear development. In addition, this isolated nucleic acid molecule may include a mutation, for example, a viral insertion, such as, one between nucleotides corresponding to nucleotides 103 and 104 of SEQ ID NO:145.

In a further aspect, the invention features an isolated nucleic acid molecule including a nucleic acid sequence having at least 80% nucleic acid sequence identity to the BAF53a nucleic acid sequence of SEQ ID NO:149 over at least 1300 contiguous nucleic acids, where this nucleic acid molecule functions in brain, body, or eye development. In addition, this isolated nucleic acid molecule may include a mutation, for example, a viral insertion, such as, one at a nucleotide corresponding to nucleotide 160 of SEQ ID NO:149.

In a further aspect, the invention features an isolated nucleic acid molecule including a nucleic acid sequence having at least 85% nucleic acid sequence identity to the Histone Deacetylase nucleic acid sequence of SEQ ID NO:151 over at least 1300 contiguous nucleic acids, where this nucleic acid molecule functions in heart, eye, ear, semicircular canal, jaw, limb, or cartilage development. In addition, this isolated nucleic acid molecule may include a mutation, for example, a viral insertion, such as, one between nucleotides corresponding to nucleotides 98 and 99 of SEQ ID NO:151, or at a nucleotide corresponding to nucleotide 88 of SEQ ID NO:151.

In a further aspect, the invention features an isolated nucleic acid molecule including a nucleic acid sequence having at least 85% nucleic acid sequence identity to the Fibroblast Isoform of the ADP/ATP Carrier Protein nucleic acid sequence of SEQ ID NO: 153 over at least 900 contiguous nucleic acids, where this nucleic acid molecule functions in lung development. In addition, this isolated nucleic acid molecule may include a mutation, for example, a viral insertion, such as, one between nucleotides corresponding to nucleotides 178 and 179 of SEQ ID NO:153.

In a further aspect, the invention features an isolated nucleic acid molecule including a nucleic acid sequence having at least 75% nucleic acid sequence identity to the TAFII-55 nucleic acid sequence of SEQ ID NO:155 over at least 600 contiguous nucleic acids, where this nucleic acid molecule functions in head, eye, or lung development. In addition, this isolated nucleic acid molecule may include a mutation, for example, a viral insertion, such as, one between nucleotides corresponding to nucleotides 107 and 108 of SEQ ID NO:155.

In a further aspect, the invention features an isolated nucleic acid molecule including a Neurogenin Related Protein-1 nucleic acid sequence of SEQ ID NO:11, where this nucleic acid molecule further includes a viral insertion at a nucleotide corresponding to nucleotide 1149 of SEQ ID NO:11.

In a sixty fourth aspect, the invention features an isolated nucleic acid molecule including a Cad-1 nucleic acid sequence of SEQ ID NO:13, where this nucleic acid molecule further includes a viral insertion between nucleotides corresponding to nucleotides 583 and 584 of SEQ ID NO:13.

In a further aspect, the invention features an isolated nucleic acid molecule including a CopZ1 nucleic acid sequence of SEQ ID NO:29, where this nucleic acid molecule further includes a viral insertion between nucleotides corresponding to nucleotides 90 and 91 of SEQ ID NO:29.

In a further aspect, the invention features an isolated nucleic acid molecule including a Ribonucleotide Reductase Protein R1 Class 1 nucleic acid sequence of SEQ ID NO:55, where this nucleic acid molecule further includes a viral insertion between nucleotides corresponding to nucleotides 147 and 148 of SEQ ID NO:55.

In a further aspect, the invention features an isolated nucleic acid molecule including an Aryl Hydrocarbon Receptor Nuclear Transporter 2A nucleic acid sequence of SEQ ID NO:63, where this nucleic acid molecule further includes a viral insertion at a nucleotide corresponding to nucleotide 229 or 240 of SEQ ID NO:63.

In a further aspect, the invention features an isolated nucleic acid molecule including a Ribonucleotide Reductase Protein R2 nucleic acid sequence of SEQ ID NO:75, where this nucleic acid molecule further includes a viral insertion at a nucleotide corresponding to nucleotide 137 of SEQ ID NO:75, or to a nucleotide corresponding to nucleotide 337 or 342 of GenBank Accession No. AW280665.

In a further aspect, the invention features an isolated nucleic acid molecule including a TCP-1 Alpha nucleic acid sequence of SEQ ID NO:77, where this nucleic acid molecule further includes a viral insertion between nucleotides corresponding to nucleotides 130 and 131 of SEQ ID NO:77, or to a nucleotide corresponding to nucleotide 140 bp upstream of nucleotide 64 of SEQ ID NO:77.

In a further aspect, the invention features an isolated nucleic acid molecule including a Cyclin A2 nucleic acid sequence of SEQ ID NO:109, where this nucleic acid molecule further includes a viral insertion at a nucleotide corresponding to nucleotide 374 or 401 of SEQ ID NO:109.

In a further aspect, the invention features an isolated nucleic acid molecule including a Sec61 Alpha nucleic acid sequence of SEQ ID NO:147, where this nucleic acid molecule further includes a viral insertion between nucleotides corresponding to nucleotides 132 and 133 of SEQ ID NO:147.

In a further aspect, the invention features an isolated polypeptide including an amino acid sequence having at least 95% sequence identity to the 904 amino acid sequence of SEQ ID NO:2 over at least 160 contiguous amino acids, where this polypeptide functions in CNS development or neural tissue proliferation.

In a further aspect, the invention features an isolated polypeptide including an amino acid sequence having at least 95% sequence identity to the U2AF amino acid sequence of SEQ ID NO:8 over at least 250 contiguous amino acids, where this polypeptide functions in brain or tectum development.

In a further aspect, the invention features an isolated polypeptide including an amino acid sequence having at least 95% sequence identity to the 954 amino acid sequence of SEQ ID NO:10, where this polypeptide functions in cartilage development.

In a further aspect, the invention features an isolated polypeptide including an amino acid sequence having at least 80% sequence identity to the V-ATPase Alpha Subunit amino acid sequence of SEQ ID NO:16 over at least 226 contiguous amino acids, where this polypeptide functions in body or eye pigmentation.

In a further aspect, the invention features an isolated polypeptide including an amino acid sequence having at least 90% sequence identity to the V-ATPase SFD Subunit amino acid sequence of SEQ ID NO:18 over at least 450 contiguous amino acids, where this polypeptide functions in body or eye pigmentation.

In a further aspect, the invention features an isolated polypeptide including an amino acid sequence having at least 95% sequence identity to the V-ATPase 16 kDa Proteolytic Subunit amino acid sequence of SEQ ID NO:20 over at least 150 contiguous amino acids, where this polypeptide functions in body or eye pigmentation, or touch sensitivity.

In a further aspect, the invention features an isolated polypeptide including an amino acid sequence having at least 50% sequence identity to the 1463 amino acid sequence of SEQ ID NO:158 over at least 475 contiguous amino acids, where this polypeptide functions in brain development, body pigmentation, or vascular development.

In a further aspect, the invention features an isolated polypeptide including an amino acid sequence having at least 70% sequence identity to the VPSP18 amino acid sequence of SEQ ID NO:22, over at least 550 contiguous amino acids, where this polypeptide functions in pigmentation, photoreceptor, retina, or tectum development.

In a further aspect, the invention features an isolated polypeptide including an amino acid sequence having at least 95% sequence identity to the 60S Ribosomal Protein L35 amino acid sequence of SEQ ID NO:26 over at least 100 contiguous amino acids, where this polypeptide functions in head, eye, or brain development.

In a further aspect, the invention features an isolated polypeptide including a sequence having at least 80% sequence identity to the 215 amino acid sequence of SEQ ID NO:32 over at least 529 contiguous amino acids, where this polypeptide functions in eye or jaw development.

In a further aspect, the invention features an isolated polypeptide including an amino acid sequence having at least 60% sequence identity to the 307 amino acid sequence of SEQ ID NO:34 over at least 200 contiguous amino acids, where this polypeptide functions in jaw or cartilage development.

In a further aspect, the invention features an isolated polypeptide including an amino acid sequence having at least 40% sequence identity to the 572 amino acid sequence of SEQ ID NO:36 over at least 200 contiguous amino acids, where this amino acid functions in jaw or cartilage development.

In a further aspect, the invention features an isolated polypeptide including an amino acid sequence having at least 45% sequence identity to the 1116A amino acid sequence of SEQ ID NO:38 over at least 200 contiguous amino acids, where this polypeptide functions in jaw development.

In a further aspect, the invention features an isolated polypeptide including an amino acid sequence having at least 76% sequence identity to the 1548 amino acid sequence of SEQ ID NO:40 over at least 950 contiguous amino acids, where this polypeptide functions in eye, head, heart, limb, jaw, or neurocranium development.

In a further aspect, the invention features an isolated polypeptide including a Casein Kinase 1α amino acid sequence of SEQ ID NO:42, where this polypeptide functions in jaw, limb, or cartilage development.

In a further aspect, the invention features an isolated polypeptide including an amino acid sequence having at least 55% sequence identity to the 429 amino acid sequence of SEQ ID NO:48 over at least 750 contiguous amino acids, where this polypeptide functions in vascular, liver, gall bladder, pancreas, or gut development.

In a further aspect, the invention features an isolated polypeptide including an amino acid sequence having at least 65% sequence identity to the 428 amino acid sequence of SEQ ID NO:50 over at least 175 contiguous amino acids, where this polypeptide functions in brain or muscle development.

In a further aspect, the invention features an isolated polypeptide including an amino acid sequence having at least 70% sequence identity to the Spinster amino acid sequence of SEQ ID NO:52 over at least 500 contiguous amino acids, where this polypeptide functions in fertility.

In a further aspect, the invention features an isolated polypeptide including an amino acid sequence having at least 60% amino acid sequence identity to the Kinesin-Related Motor Protein EG5 amino acid sequence of SEQ ID NO:58 over at least 900 contiguous amino acids, where this polypeptide functions in cell death regulation or body development.

In a further aspect, the invention features an isolated polypeptide including an amino acid sequence having at least 75% sequence identity to the 459 amino acid sequence of SEQ ID NO:60 over at least 250 contiguous amino acids, where this polypeptide functions in body development or cell death regulation.

In a further aspect, the invention features an isolated polypeptide including an amino acid sequence having at least 65% sequence identity to the Vesicular Integral-Membrane Protein VIP 36 amino acid sequence of SEQ ID NO:66 over at least 320 contiguous amino acids, where this polypeptide functions in touch sensitivity.

In a further aspect, the invention features an isolated polypeptide including an amino acid sequence having at least 50% sequence identity to the 299 amino acid sequence of SEQ ID NO:68 over at least 500 contiguous amino acids, where this polypeptide functions in cell death regulation in the eye or brain, or in jaw, cartilage, or limb development.

In a further aspect, the invention features an isolated polypeptide including an amino acid sequence having at least 40% sequence identity to the 994 amino acid sequence of SEQ ID NO:70 over at least 500 contiguous amino acids, where this polypeptide functions in eye, head, jaw, cartilage, or stomach development.

In a further aspect, the invention features an isolated polypeptide including at least 110 contiguous amino acids of the 1373 amino acid sequence of SEQ ID NO:72, where this polypeptide functions in brain, eye, or body development.

In a further aspect, the invention features an isolated polypeptide including an amino acid sequence having at least 70% sequence identity to the Denticleless amino acid sequence of SEQ ID NO:74 over at least 400 contiguous amino acids, where this amino acid functions in CNS, body, yolk sac, somite, or eye development.

In a further aspect, the invention features an isolated polypeptide including an amino acid sequence having at least 35% sequence identity to the Telomeric Repeat Factor 2 amino acid sequence of SEQ ID NO:80 over at least 200 contiguous amino acids, where this polypeptide functions in brain or eye development.

In a further aspect, the invention features an isolated polypeptide including an amino acid sequence having at least 40% sequence identity to the SIL amino acid sequence of SEQ ID NO:82 over at least 1200 contiguous amino acids, where this polypeptide functions in brain, head, or body development, or in motility.

In a further aspect, the invention features an isolated polypeptide including an amino acid sequence having at least 87% sequence identity to the U1 the Small Nuclear Ribonucleoprotein C polypeptide of SEQ ID NO:84 over at least 150 contiguous amino acids, where this polypeptide functions in body, eye, hindbrain, ear, pigment, or limb development.

In a further aspect, the invention features an isolated polypeptide including an amino acid sequence having at least 87% sequence identity to the Ski Interacting Polypeptide amino acid sequence of SEQ ID NO:86 over at least 500 contiguous amino acids, where this polypeptide functions in body or brain development, or in motility.

In a further aspect, the invention features an isolated polypeptide including an amino acid sequence having at least 80% sequence identity to the 297 amino acid sequence of SEQ ID NO:88 over at least 600 contiguous amino acids, where this polypeptide functions in motility, cartilage, cranium, eye, tail, or brain development.

In a further aspect, the invention features an isolated polypeptide including an amino acid sequence having at least 90% sequence identity to the TCP-1 Complex Gamma Chain amino acid sequence of SEQ ID NO:90 over at least 500 contiguous amino acids, where this polypeptide functions in yolk sac development.

In a further aspect, the invention features an isolated polypeptide including an amino acid sequence having at least 99% sequence identity to Small Nuclear Ribonucleoprotein D1 amino acid sequence of SEQ ID NO:92, where this polypeptide functions in CNS or eye development.

In a further aspect, the invention features an isolated polypeptide including an amino acid sequence having at least 76% sequence identity to the DNA polymerase Epsilon Subunit B amino acid sequence of SEQ ID NO:94 over at least 500 contiguous amino acids, where this polypeptide functions in brain or eye development.

In a further aspect, the invention features an isolated polypeptide including a amino acid sequence having at least 60% sequence identity over the full length of the 821-02 amino acid sequence of SEQ ID NO:96; where this polypeptide functions in cell death regulation in the CNS or the eye.

In a further aspect, the invention features an isolated polypeptide including an amino acid sequence having at least 78% sequence identity to the 1045 amino acid sequence of SEQ ID NO:98 over at least 300 contiguous amino acids, where this polypeptide functions in brain or head development.

In a further aspect, the invention features an isolated polypeptide including an amino acid sequence having at least 75% sequence identity to the 1055-1 amino acid sequence of SEQ ID NO:100 over at least 300 contiguous amino acids, where this polypeptide functions in cell cycle progression or 60S ribosomal subunit biogenesis.

In a further aspect, the invention features an isolated polypeptide including an amino acid sequence having at least 85% sequence identity to the Spliceosome Associated Protein 49 polypeptide of amino acid sequence SEQ ID NO:102 over at least 350 contiguous amino acids, where this polypeptide functions in tectal or body development.

In a further aspect, the invention features an isolated polypeptide including an amino acid sequence having at least 80% sequence dentity to the DNA Replication Licensing factor MCM7 amino acid sequence of SEQ ID NO:104 over at least 190 contiguous amino acids, where this polypeptide functions in eye or CNS development.

In a further aspect, the invention features an isolated polypeptide including an amino acid sequence having at least 85% sequence identity to the DEAD-Box RNA Helicase (DEAD5 or DEAD19) amino acid sequence of SEQ ID NO:106 over at least 450 contiguous amino acids, where this polypeptide functions in brain development.

In a further aspect, the invention features an isolated polypeptide including a amino acid acid sequence having at least 50% sequence identity to the 1581 amino acid sequence of SEQ ID NO:108 over at least 300 contiguous amino acids, where this polypeptide functions in head or eye development.

In a further aspect, the invention features an isolated polypeptide including an amino acid acid sequence having at least 75% sequence identity to Imitation Switch (ISWI)/SNF2 amino acid sequence of SEQ ID NO:112 over at least 150 contiguous amino acids, where this polypeptide functions in development of the visual system or of cartilage.

In a further aspect, the invention features an isolated polypeptide including an amino acid sequence having at least 75% sequence identity over the full length of the Chromosomal Assembly Protein C (XCAP-C) amino acid sequence of SEQ ID NO:114, where this polypeptide functions in optic tectum, eye, or hindbrain development.

In a further aspect, the invention features an isolated polypeptide including an amino acid sequence having at least 83% sequence identity over the full length of the DNA Replication Licensing Factor MCM2 amino acid sequence of SEQ ID NO:116, where this polypeptide functions in eye, optic tectum, jaw, or cartilage development.

In a further aspect, the invention features an isolated polypeptide including an amino acid sequence having at least 88% sequence identity to the DNA Replication Licensing Factor MCM3 amino acid sequence of SEQ ID NO:118 over at least 170 contiguous amino acids, where this polypeptide functions in optic tectum, head, or eye development.

In a further aspect, the invention features an isolated polypeptide including an amino acid sequence having at least 55% sequence identity to the Valyl-tRNA synthase amino acid sequence of SEQ ID NO:120 over at least 450 contiguous amino acids, where this polypeptide functions in cell death regulation, head or eye development, or pigmentation.

In a further aspect, the invention features an isolated polypeptide including an amino acid sequence having at least 98% sequence identity to the 40S Ribosomal Protein S5 amino acid sequence of SEQ ID NO:122 over at least 210 contiguous amino acids, where this polypeptide functions in brain development or motility.

In a further aspect, the invention features an isolated polypeptide including an amino acid sequence having at least 90% sequence identity to the TCP-1 Beta amino acid sequence of SEQ ID NO:124 over at least 500 contiguous amino acids, where this polypeptide functions in jaw, head, eye, or cartilage development.

In a further aspect, the invention features an isolated polypeptide including an amino acid sequence having at least 90% sequence identity to the TCP-1 Eta amino acid sequence of SEQ ID NO:126 over at least 500 contiguous amino acids, where this polypeptide functions in head and eye development.

In a further aspect, the invention features an isolated polypeptide including the amino acid sequence of Translation Elongation Factor eEF Alpha amino acid sequence of SEQ ID NO:128, where this polypeptide functions in cell death regulation in the head or eye.

In a further aspect, the invention features an isolated polypeptide including an amino acid sequence having at least 55% sequence identity to the 1257 amino acid sequence of SEQ ID NO:130 over at least 350 contiguous amino acids, where this polypeptide functions in head, eye, or jaw development.

In a further aspect, the invention features an isolated polypeptide including an amino acid sequence having at least 90% sequence identity to the 60S Ribosomal Protein L24 amino acid sequence of SEQ ID NO:132 over at least 160 contiguous amino acid, where this polypeptide functions in head or eye development.

In a further aspect, the invention features an isolated polypeptide including a amino acid sequence having at least 80% sequence identity to the Non-Muscle Adenylosuccinate. Synthase amino acid sequence of SEQ ID NO:134, where this polypeptide functions in cell death regulation, or in jaw or cartilage development.

In a further aspect, the invention features an isolated polypeptide including an amino acid sequence having at least 90% sequence identity to the Nuclear Cap Binding Protein Subunit 2 amino acid sequence of SEQ ID NO:136, over at least 150 contiguous amino acids, where this polypeptide functions in head, eye, CNS, jaw, cartilage, or stomach development.

In a further aspect, the invention features an isolated polypeptide including the Omithine Decarboxylase amino acid sequence of SEQ ID NO:138, where this polypeptide functions in jaw or cartilage development.

In a further aspect, the invention features an isolated polypeptide including a amino acid sequence having at least 60% sequence identity to the Protein Phosphatase 1 Nuclear Targeting Subunit amino acid sequence of SEQ ID NO:140 over at least 600 contiguous amino acids, where this polypeptide functions in head, jaw, body, or gut development.

In a further aspect, the invention features an isolated polypeptide including an amino acid sequence having at least 80% sequence identity to the Mitochondrial Inner Membrane Translocating (rTIM23) amino acid sequence of SEQ ID NO:142 over at least 175 contiguous amino acids, where this polypeptide functions in the development of the vascular system or in eye pigmentation.

In a further aspect, the invention features an isolated polypeptide including an amino acid sequence having at least 65% sequence identity to the 1447 amino acid sequence of SEQ ID NO:144 over at least 700 contiguous amino acids, where this polypeptide functions in pancreas, tail, stomach, or limb development.

In a further aspect, the invention features an isolated polypeptide including an amino acid sequence having at least 72% sequence identity to the ARS2 amino acid sequence of SEQ ID NO:146 over at least 900 contiguous amino acids, where this polypeptide functions in pigment, tectum, cartilage, jaw, or ear development.

In a further aspect, the invention features an isolated polypeptide including an amino acid sequence having at least 90% sequence identity over the full length of the BAF53a amino acid sequence of SEQ ID NO:150, where this polypeptide functions in brain, body, or eye development.

In a further aspect, the invention features an isolated polypeptide including an amino acid sequence having at least 92% sequence identity to the Histone Deacetylase amino acid sequence of SEQ ID NO:152, where this polypeptide functions in heart, eye, ear, semicircular canal, jaw, limb, or cartilage development.

In a further aspect, the invention features an isolated polypeptide including an amino acid sequence having at least 95% sequence identity to the Fibroblast Isoform of the ADP/ATP Carrier protein amino acid sequence of SEQ ID NO:154 over at least 300 contiguous amino acids, where this polypeptide functions in lung development.

In a further aspect, the invention features an isolated polypeptide including an amino acid sequence having at least 70% sequence identity to the TAFII-55 amino acid sequence of SEQ ID NO:156, where this polypeptide functions in head, lung, or eye development.

In a further aspect, the invention features an isolated nucleic acid (i) encoding the amino acid sequence of SEQ ID NO:2, 8, 10, 16, 18, 20, 158, 22, 26, 32, 34, 36, 38, 40, 42, 48, 50, 52, 58, 60, 66, 68, 70, 72, 74, 80, 82, 84, 86, 88, 90, 92, 94, 96, 98, 100, 102, 104, 106, 108, 112, 114, 116, 118, 120, 122, 124, 126, 128, 130, 132, 134, 136, 138, 140, 142, 144, 146, 150, 152, 154, or 156; (ii) an isolated nucleic acid molecule having the nucleic acid sequence of SEQ ID NO:1, 7, 9, 15, 17, 19, 157, 21, 25, 31, 33, 35, 37, 39, 41, 47, 49, 51, 57, 59, 65, 67, 69, 71, 73, 79, 81, 83, 85, 87, 89, 91, 93, 95, 97, 99, 101, 103, 105, 107, 111, 113, 115, 117, 119, 121, 123, 125, 127, 129, 131, 133, 135, 137, 139, 141, 143, 145, 149, 151, 153, or 155; (iii) an isolated nucleic acid molecule that hybridizes under highly stringent conditions to a probe having the nucleic acid sequence of SEQ ID NO:1, 7, 9, 15, 17, 19, 157, 21, 25, 31, 33, 35, 37, 39, 41, 47 49, 51, 57, 59, 65, 67, 69, 71, 73, 79, 81, 83, 85, 87, 89, 91, 93, 95, 97, 99, 101, 103, 105, 107, 111, 113, 115, 117, 119, 121, 123, 125, 127, 129, 131, 133, 135, 137, 139, 141, 143, 145, 149, 151, 153, or 155 or a portion thereof; or (iv) an isolated nucleic acid molecule complementary to the isolated nucleic acid molecule of (i), (ii), or (iii). For example, the nucleic acid molecule may be a vertebrate, e.g., human, mouse, or zebrafish nucleic acid molecule. In addition, the invention also features a vector including an isolated nucleic acid molecule of the invention, for example, one operably linked to a promoter, as well as a cell including such a vector.

In a further aspect, the invention features an isolated polypeptide including the amino acid sequence of SEQ ID NO:2, 8, 10, 16, 18, 20, 158, 22, 26, 32, 34, 36, 38, 40, 42, 48, 50, 52, 58, 60, 66, 68, 70, 72, 74, 80, 82, 84, 86, 88, 90, 92, 94, 96, 98, 100, 102, 104, 106, 108, 112, 114, 116, 118, 120, 122, 124, 126, 128, 130, 132, 134, 136, 138, 140, 142, 144, 146, 150, 152, 154, or 156; having the amino acid sequence of SEQ ID NO:2, 8, 10, 16, 18, 20, 158, 22, 26, 32, 34, 36, 38, 40, 42, 48, 50, 52, 58, 60, 66, 68, 70, 72, 74, 80, 82, 84, 86, 88, 90, 92, 94, 96, 98, 100, 102, 104, 106, 108, 112, 114, 116, 118, 120, 122, 124, 126, 128, 130, 132, 134, 136, 138, 140, 142, 144, 146, 150, 152, 154, or 156; or a polypeptide that is encoded by an isolated nucleic acid molecule including the nucleic acid sequence of SEQ ID NO:1, 7, 9, 15, 17, 19, 157, 21, 25, 31, 33, 35, 37, 39, 41, 47, 49, 51, 57, 59, 65, 67, 69, 71, 73, 79, 81, 83, 85, 87, 89, 91, 93, 95, 97, 99, 101, 103, 105, 107, 111, 113, 115, 117, 119, 121, 123, 125, 127, 129, 131, 133, 135, 137, 139, 141, 143, 145, 149, 151, 153, or 155.

In a further aspect, the invention features a method of treating or preventing a proliferative disorder in an organism, the method including the step of contacting the organism with a therapeutically effective amount of a nucleic acid including the nucleic acid sequence of SEQ ID NO:1, 57, 59, 67, 95, 99, 119, or 127 its complement, where the nucleic acid is sufficient to elicit an alteration in expression of a 904, 1055-1, Kinesin Related Motor Protein EG5, 459, 299, Valyl-tRNA Synthase, Translation Elongation Factor eEF1 Alpha, 821-02 nucleic acid sequence in the organism, and where this alteration in the level of expression treats or prevents a proliferative disorder. In addition, this nucleic acid may be, for example, a cDNA or an mRNA molecule and the contacting may result in an increase in expression of the polypeptide encoded by the nucleic acid sequence of SEQ ID NO:1, 57, 59, 67, 95, 99, 119, or 127. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:1, 57, 59, 67, 95, 99, 119, or 127.

In a further aspect, the invention features a method of treating or preventing a bone, connective tissue, or cartilage disorder in an organism, the method including the step of contacting the organism with a therapeutically effective amount of a nucleic acid including the nucleic acid sequence of SEQ ID NO:9, 31, 33, 35, 37, 39, 41, 67, 69, 123, 133, 135, 137, 143, or 151 or its complement, where the nucleic acid is sufficient to elicit an alteration in expression of a 954, histone deacetylase, 215, 307, 572, 1116A, 1548, Casein kinase 1α, 299, 994, TCP-1 Beta, Non-Muscle Adenylosuccinate Synthase, Nuclear Cap Binding Protein Subunit 2, Ornithine Decarboxylase, or 1447 nucleic acid sequence in the organism, and where this alteration in the level of expression treats or prevents a bone, connective tissue, or cartilage disorder. In addition, the nucleic acid may be, for example, a cDNA or an mRNA molecule and the contacting may result in an increase in expression of the polypeptide encoded by the nucleic acid sequence of SEQ ID NO:9, 31, 33, 35, 37, 39, 41, 67, 69, 123, 133, 135, 137, 143, or 151. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:9, 31, 33, 35, 37, 39, 41, 67, 69, 123, 133, 135, 137, 143, or 151.

In a further aspect, the invention features a method of treating or preventing a cell death disorder in an organism, the method including the step of contacting the organism with a therapeutically effective amount of a nucleic acid including the nucleic acid sequence of SEQ ID NO:7, 57, 59, 67, 71, 73, 79, 81, 83, 85, 87, 91, 93, 95, 97, 101, 103, 105, 107, 111, 113, 115, 117, 119, 127, 133, 135, 137, 139, or 145 or its complement, where the nucleic acid is sufficient to elicit an alteration in expression of a U2AF, Kinesin Related Motor Protein EG5, 459, 299, 1373, Denticleless, Telomeric Repeat Factor 2, SIL, U1 Small Nuclear Ribonuclear Protein C, Ski Interacting Protein, 297, Small Nuclear Ribonucleoprotein D1, DNA Polymerase Epsilon Subunit B, 1045, Spliceosome Associated Protein 49, DNA Replication Licensing Factor MCM7, 1581, DEAD-Box RNA Helicase (DEAD5 or DEAD19), ISWI/SNF2, XCAP-C, DNA Replication Licensing Factor MCM2, DNA Replication Licensing Factor MCM3, Nuclear Cap Binding Protein Subunit 2, Ornithine Decarboxylase, ARS2, Protein Phosphatase 1 Nuclear Targeting Subunit, Valyl-tRNA Synthase, Translation Elongation Factor eEF1 Alpha, Non-Muscle Adenylosuccinate Synthase, or 821-02 nucleic acid sequence in the organism, and where this alteration in the level of expression treats or prevents a cell death disorder. In addition, the nucleic acid may be, for example, a cDNA or an mRNA molecule and the contacting may result in an increase in expression of the polypeptide encoded by the nucleic acid sequence of SEQ ID NO:7, 57, 59, 67, 71, 73, 79, 81, 83, 85, 87, 91, 93, 95, 97, 101, 103, 105, 107, 111, 113, 115, 117, 119, 127, 133, 135, 137, 139, or 145. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:7, 57, 59, 67, 71, 73, 79, 81, 83, 85, 87, 91, 93, 95, 97, 101, 103, 105, 107, 111, 113, 115, 117, 119, 127, 133, 135, 137, 139, or 145.

In a further aspect, the invention features a method of treating or preventing a circulatory disorder in an organism, the method including the step of contacting the organism with a therapeutically effective amount of a nucleic acid including the nucleic acid sequence of SEQ ID NO:1, 47, 141, or 157 or its complement, where the nucleic acid is sufficient to elicit an alteration in expression of a 904, 1463, 429, or Mitochondrial Inner Membrane Translocating (rTIM23) nucleic acid sequence in the organism, and where this alteration in the level of expression treats or prevents a circulatory disorder. In addition, the nucleic acid may be, for example, a cDNA or an mRNA molecule and the contacting may result in an increase in expression of the polypeptide encoded by the nucleic acid sequence of SEQ ID NO:1, 47, 141, or 157. Alternatively, the nucleic acid may be a double-stranded RNA molecule, or an anti-sense RNA molecule, and the contacting may lead to a decrease in expression, or may inhibit biological activity, of a nucleic acid sequence including SEQ ID NO:1, 47, 141, or 157.

In a further aspect, the invention features a method of treating or preventing a craniofacial defect in an organism, the method including the step of contacting the organism with a therapeutically effective amount of a nucleic acid including the nucleic acid sequence of SEQ ID NO:31, 33, 35, 37, 39, 41, 67, 87, 115, 123, 129, 133, 135, or 137 or its complement, where the nucleic acid is sufficient to elicit an alteration in expression of a 215, 307, 572, 1116A, 1548, Casein Kinase 1α, 299, 297, DNA Replication Licensing Factor MCM2, 1257, TCP-1 Beta, Nuclear Cap Binding Protein Subunit 2, Non-Muscle Adenylosuccinate Synthase, or Ornithine Decarboxylase nucleic acid sequence in the organism, and where this alteration in the level of expression treats or prevents a craniofacial defect. In addition, the nucleic acid may be, for example, a cDNA or an mRNA molecule and the contacting may result in an increase in expression of the polypeptide encoded by the nucleic acid sequence of SEQ ID NO:31, 33, 35, 37, 39, 41, 67, 87, 115, 123, 129, 133, 135, or 137. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:31, 33, 35, 37, 39, 41, 67, 87, 115, 123, 129, 133, 135, or 137.

In a further aspect, the invention features a method of treating or preventing a hearing disorder in an organism, the method including the step of contacting the organism with a therapeutically effective amount of a nucleic acid including the nucleic acid sequence of SEQ ID NO:83, 125, 145, or 151 or its complement, where the nucleic acid is sufficient to elicit an alteration in expression of a U1 Small Nuclear Ribonucleoprotein C, ARS2, TCP-1 Eta, or Histone Deacetylase nucleic acid sequence in the organism, and where this alteration in the level of expression treats or prevents a hearing disorder. In addition, the nucleic acid may be, for example, a cDNA or an mRNA molecule and the contacting may result in an increase in expression of the polypeptide encoded by the nucleic acid sequence of SEQ ID NO:83, 125, 145, or 151. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:83, 125, 145, or 151.

In a further aspect, the invention features a method of treating or preventing diabetes in an organism, the method including the step of contacting the organism with a therapeutically effective amount of a nucleic acid including the nucleic acid sequence of SEQ ID NO:47 or 143 or its complement, where the nucleic acid is sufficient to elicit an alteration in expression of a 429 or 1447 nucleic acid sequence in the organism, and where this alteration in the level of expression treats or prevents diabetes. In addition, the nucleic acid may be, for example, a cDNA or an mRNA molecule and the contacting results in an increase in expression of the polypeptide encoded by the nucleic acid sequence of SEQ ID NO:47 or 143. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:47 or 143.

In a further aspect, the invention features a method of treating or preventing a heart defect, disease, or disorder in an organism, the method including the step of contacting the organism with a therapeutically effective amount of a nucleic acid including the nucleic acid sequence of SEQ ID NO:39, 77, 135, 141, or 151 or its complement, where the nucleic acid is sufficient to elicit an alteration in expression of a 1548, Histone Deacetylase, or Nuclear Cap Binding Protein Subunit 2, Mitochondrial Inner Membrane Translocating Protein (rTIM23), or TCP-1 Alpha nucleic acid sequence in the organism, and where this alteration in the level of expression treats or prevents a heart defect, disease, or disorder. In addition, the nucleic acid may be, for example, a cDNA or an mRNA molecule and the contacting may result in an increase in expression of the polypeptide encoded by the nucleic acid sequence of SEQ ID NO:25, 39, 77, 135, 141, or 151. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:25, 39, 77, 135, 141, or 151.

In a further aspect, the invention features a method of treating or preventing infertility in an organism, the method including the step of contacting the organism with a therapeutically effective amount of a nucleic acid including the nucleic acid sequence of SEQ ID NO:51 or its complement, where the nucleic acid is sufficient to elicit an alteration in expression of a Spinster nucleic acid sequence in the organism, and where this alteration in the level of expression treats or prevents infertility. In addition, the nucleic acid may be, for example, a cDNA or an mRNA molecule and the contacting may result in an increase in expression of the polypeptide encoded by the nucleic acid sequence of SEQ ID NO:51. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:51.

In a further aspect, the invention features a method of treating or preventing a limb formation defect in an organism, the method including the step of contacting the organism with a therapeutically effective amount of a nucleic acid including the nucleic acid sequence of SEQ ID NO:41 or 151 or its complement, where the nucleic acid is sufficient to elicit an alteration in expression of a Casein Kinase 1α or Histone Deacetylase nucleic acid sequence in the organism, and where this alteration in the level of expression treats or prevents a limb formation defect. In addition, the nucleic acid may be, for example, a cDNA or an mRNA molecule and the contacting may result in an increase in expression of the polypeptide encoded by the nucleic acid sequence of SEQ ID NO:41 or 151. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:41 or 151.

In a further aspect, the invention features a method of treating or preventing mental retardation in an organism, the method including the step of contacting the organism with a therapeutically effective amount of a nucleic acid including the nucleic acid sequence of SEQ ID NO:7, 21, 25, 67, 69, 71, 79, 81, 83, 85, 89, 91, 93, 97, 101, 103, 105, 107, 111, 113, 115, 117, 119, 121, 123, 127, 129, 131, 133, 135, 137, 139, 143, 145, 149, 151, 155, or 157 or its complement, where the nucleic acid is sufficient to elicit an alteration in expression of a U2AF, 1463, VPSP18, 60S Ribosomal Protein L35, 299, 1373, Telomeric Repeat Factor 2, SIL, U1 Small Ribonucleoprotein C, SKIP, 297, Small Nuclear Ribonucleoprotein D1, DNA Polymerase Epsilon Subunit B, 1045, Spliceosome Associated Protein 49, DNA Replication Licensing Factor MCM7, DEAD-Box RNA Helicase (DEAD5 or DEAD19), 1581, ISWI/SNF, Chromosomal Assembly Protein (XCAP-C), DNA Replication Licensing Factor MCM2, DNA Replication Licensing Factor MCM3, Valy-tRNA Synthase, 40S Ribosomal. Protein S5, TCP-1 Alpha, TCP-1 Beta, Translation Elongation Factor eEF1 Alpha, 1257, 60S Ribosomal Protein L24, Non-Muscle Adenylosuccinate Synthase, Nuclear Cap Binding Protein Subunit 2, ARS2, BAF53a, Histone Deacetylase, Protein Phosphatase 1 Nuclear Targeting Subunit protein (PNUTS), Ornithine Decarboxylase, 1447, 1262, 994, or TAFII-55 nucleic acid sequence in the organism, and where this alteration in the level of expression treats or prevents mental retardation. In addition, the nucleic acid may be a cDNA or an mRNA molecule and the contacting may result in an increase in expression of the polypeptide encoded by the nucleic acid sequence of SEQ ID NO:7, 21, 25, 67, 69, 71, 79, 81, 83, 85, 89, 91, 93, 97, 101, 103, 105, 107, 111, 113, 115, 117, 119, 121, 123, 127, 129, 131, 133, 135, 137, 139, 143, 145, 149, 151, 155, or 157. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:7, 21, 25, 67, 69, 71, 79, 81, 83, 85, 89, 91, 93, 97, 101, 103, 105, 107, 111, 113, 115, 117, 119, 121, 123, 127, 129, 131, 133, 135, 137, 139, 143, 145, 149, 151, 155, or 157.

In a further aspect, the invention features a method of treating or preventing a muscle defect in an organism, the method including the step of contacting the organism with a therapeutically effective amount of a nucleic acid including the nucleic acid sequence of SEQ ID NO:49 or 73 or its complement, where the nucleic acid is sufficient to elicit an alteration in expression of a 428 or Denticleless nucleic acid sequence in the organism, and where this alteration in the level of expression treats or prevents a muscle defect. In addition, the nucleic acid may be, for example, a cDNA or an mRNA molecule and the contacting may result in an increase in expression of the polypeptide encoded by the nucleic acid sequence of SEQ ID NO:49 or 73. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:49 or 73.

In a further aspect, the invention features a method of treating or preventing a neurodegenerative disorder in an organism, the method including the step of contacting the organism with a therapeutically effective amount of a nucleic acid including the nucleic acid sequence of SEQ ID NO:19, 57, 59, 65, 67, 87, 95, 119, 121, or 127 or its complement, where the nucleic acid is sufficient to elicit an alteration in expression of a Vesicular Integral Membrane Protein VIP 36, 297, 40S Ribosomal Protein S5, V-ATPase 16 kDa Proteolytic Subunit, Kinesin Related Motor Protein EG5, 459, 299, 821-02, Valyl-tRNA Synthase, or Translation Elongation Factor eEF1 Alpha nucleic acid sequence in the organism, and where this alteration in the level of expression treats or prevents a neurodegenerative disorder. In addition, the nucleic acid may be, for example, a cDNA or an mRNA molecule and the contacting may result in an increase in expression of the polypeptide encoded by the nucleic acid sequence of SEQ ID NO:19, 57, 65, 67, 69, 87, 95, 119, 121, or 127. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:19, 57, 59, 65, 67, 87, 95, 119, 121, or 127.

In a further aspect, the invention features a method of treating or preventing stroke in an organism, the method including the step of contacting the organism with a therapeutically effective amount of a nucleic acid including the nucleic acid sequence of SEQ ID NO:1, 141, or 157 or its complement, where the nucleic acid is sufficient to elicit an alteration in expression of a 904, Mitochondrial Inner Membrane Translocating (rTIM23), or 1463 nucleic acid sequence in the organism, and where this alteration in the level of expression treats or prevents stroke. In addition, the nucleic acid may be, for example, a cDNA or an mRNA molecule and the contacting may result in an increase in expression of the polypeptide encoded by the nucleic acid sequence of SEQ ID NO:1, 141, or 157. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:1, 141, or 157.

In a further aspect, the invention features a method of treating or preventing a stem cell regeneration disorder in an organism, the method including the step of contacting the organism with a therapeutically effective amount of a nucleic acid including the nucleic acid sequence of SEQ ID NO:1, 141, or 143 or its complement, where the nucleic acid is sufficient to elicit an alteration in expression of a 904, Mitochondrial Inner Membrane Translocating (rTIM23), or 1447 nucleic acid sequence in the organism, and where this alteration in the level of expression treats or prevents a stem cell regeneration disorder. In addition, the nucleic acid may be, for example, a cDNA or an mRNA molecule and the contacting may result in an increase in expression of the polypeptide encoded by the nucleic acid sequence of SEQ ID NO:1, 141, or 143. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:1, 141, or 143.

In a further aspect, the invention features a method of treating or preventing a visual defect in an organism, the method including the step of contacting the organism with a therapeutically effective amount of a nucleic acid including the nucleic acid sequence of SEQ ID NO:15, 17, 19, 21, 55, 79, 81, 87, 111, or 157 or its complement, where the nucleic acid is sufficient to elicit an alteration in expression of a V-ATPase SFD subunit, V-ATPase 16 kDa Proteolytic Subunit, V-ATPase Alpha Subunit, 1463, VPSP8, Ribonucleotide Reductase R1 Class 1, Telomeric Repeat Factor 2, SIL, ISWI/SNF2 or 297 nucleic acid sequence in the organism, and where this alteration in the level of expression treats or prevents a visual defect. In addition, the nucleic acid may be, for example, a cDNA or an mRNA molecule and the contacting may result in an increase in expression of the polypeptide encoded by the nucleic acid sequence of SEQ ID NO:15, 17, 19, 21, 55, 79, 81, 87, 111, or 157. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:15, 17, 19, 21, 55, 79, 81, 87, 111, or 157.

In a further aspect, the invention features a method of treating or preventing a pulmonary disorder in an organism, the method including the step of contacting the organism with a therapeutically effective amount of a nucleic acid including the nucleic acid sequence of SEQ ID NO:153 or 155 or its complement, where the nucleic acid is sufficient to elicit an alteration in expression of an Fibroblast Isoform of the ADP/ATP Carrier Protein or TAFII-55 nucleic acid sequence in the organism, and where this alteration in the level of expression treats or prevents a pulmonary disorder. In addition, the nucleic acid may be, for example, a cDNA or an mRNA molecule and the contacting may result in an increase in expression of the polypeptide encoded by the nucleic acid sequence of SEQ ID NO:153 or 155. Alternatively, the nucleic acid may be a double-stranded RNA or an anti-sense RNA molecule, and the contacting may lead to a decrease in expression, or may inhibit biological activity, of a nucleic acid sequence including SEQ ID NO:153 or 155.

In a further aspect, the invention features a method of treating or preventing a movement disorder in an organism, the method including the step of contacting the organism with a therapeutically effective amount of a nucleic acid including the nucleic acid sequence of SEQ ID NO:11, 13, 63, 81, 83, 85, 121 or its complement, where the nucleic acid is sufficient to elicit an alteration in expression of a Cad-1, Aryl Hydrocarbon Receptor Nuclear Translocator 2A, or 40S Ribosomal Protein S5, Neurogenin Related Protein 1, SIL, U1 Small Nuclear Ribonucleoprotein C, Ski Interacting Protein nucleic acid sequence in the organism, and where this alteration in the level of expression treats or prevents a movement disorder. In addition, the nucleic acid may be, for example, a cDNA or an mRNA molecule and the contacting may result in an increase in expression of the polypeptide encoded by the nucleic acid sequence of SEQ ID NO:11, 13, 63, 81, 83, 85, or 121. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:11, 13, 63, 81, 83, 85, or 121.

In a further aspect, the invention features a method of treating or preventing a somite formation disorder in an organism, the method including the step of contacting the organism with a therapeutically effective amount of a nucleic acid including the nucleic acid sequence of SEQ ID NO:49 or 73 or its complement, where the nucleic acid is sufficient to elicit an alteration in expression of a 428 or Denticleless nucleic acid sequence in the organism, and where this alteration in the level of expression treats or prevents a somite formation disorder. In addition, the nucleic acid may be, for example, a cDNA or an mRNA molecule and the contacting may result in an increase in expression of the polypeptide encoded by the nucleic acid sequence of SEQ ID NO:49 or 73. Alternatively, the nucleic acid may be a double-stranded RNA molecule or an anti-sense RNA molecule, and the contacting may lead to a decrease in expression, or may inhibit biological activity, of a nucleic acid sequence including SEQ ID NO:49 or 73.

In a further aspect, the invention features a method for diagnosing a proliferative disorder or the propensity to develop a proliferative disorder in an organism. This method includes detecting an alteration in the level of 904, 1055-1, Kinesin Related Motor Protein EG5, 459, 299, Valyl-tRNA Synthase, Translation Elongation Factor eEF1 Alpha, 821-02 polypeptide expression in a sample derived from a first organism and comparing the level of expression to that of a 904, 1055-1, Kinesin Related Motor Protein EG5, 459, 299, Valyl-tRNA Synthase, Translation Elongation Factor eEF1 Alpha, 821-02 polypeptide in a sample derived from a second, control organism, where an alteration in the level of expression or activity of the 904, 1055-1, Kinesin Related Motor Protein EG5, 459, 299, Valyl-tRNA Synthase, Translation Elongation Factor eEF1 Alpha, 821-02 polypeptide in the first organism relative to the second organism is indicative of the first organism having or having a propensity to develop a proliferative disorder. In a further aspect, the invention features a method for diagnosing a proliferative disorder or the propensity to develop a proliferative disorder in an organism. This method includes detecting an alteration in the sequence, or a portion of the sequence, of a 904, 1055-1, Kinesin Related Motor Protein EG5, 459, 299, Valyl-tRNA Synthase, Translation Elongation Factor eEF1 Alpha, 821-02 nucleic acid molecule in a sample derived from a first organism and comparing the sequence to that of a 904, 1055-1, Kinesin Related Motor Protein EG5, 459, 299, Valyl-tRNA Synthase, Translation Elongation Factor eEF1 Alpha, 821-02 nucleic acid molecule derived from a second, control organism, where an alteration of the 904, 1055-1, Kinesin Related Motor Protein EG5, 459, 299, Valyl-tRNA Synthase, Translation Elongation Factor eEF1 Alpha, 821-02 sequence in the first organism relative to the second organism is indicative of the first organism having or having a propensity to develop a proliferative disorder. In the alternative, the alteration may be an increase or a decrease in the expression or activity of a nucleic acid molecule including the sequence of SEQ ID NO:1, 57, 59, 67, 95, 99, 119, or 127.

In a further aspect, the invention features a method for identifying a candidate compound for the treatment of a proliferative disorder. This method includes the steps of: (a) contacting a polypeptide including the amino acid sequence of SEQ ID NO:2, 58, 60, 68, 96, 100, 120, or 128 with a candidate compound, and (b) detecting an interaction of the polypeptide with the candidate compound, where a candidate compound that binds the polypeptide may be used to treat a proliferative disorder.

In a further aspect, the invention features a method for diagnosing a bone, connective tissue, or cartilage formation disorder or the propensity to develop a bone, connective tissue, or cartilage formation disorder in an organism. This method includes detecting an alteration in the level of 954, histone deacetylase, 215, 307, 572, 1116A, 1548, Casein kinase 1α, 299, 994, TCP-1 Beta, Non-Muscle Adenylosuccinate Synthase, Nuclear Cap Binding Protein Subunit 2, Ornithine Decarboxylase, or 1447 polypeptide expression in a sample derived from a first organism and comparing the level of expression to that of a 954, histone deacetylase, 215, 307, 572, 1116A, 1548, Casein kinase 1α, 299, 994, TCP-1 Beta, Non-Muscle Adenylosuccinate Synthase, Nuclear Cap Binding Protein Subunit 2, Ornithine Decarboxylase, or 1447 polypeptide in a sample derived from a second, control organism, where an alteration in the level of expression or activity of 954, histone deacetylase, 215, 307, 572, 1116A, 1548, Casein kinase 1α, 299, 994, TCP-1 Beta, Non-Muscle Adenylosuccinate Synthase, Nuclear Cap Binding Protein Subunit 2, Omithine Decarboxylase, or 1447 polypeptide in the first organism relative to the second organism is indicative of the first organism having or having a propensity to develop a bone, connective tissue, or cartilage formation disorder.

In a further aspect, the invention features a method for diagnosing a bone, connective tissue, or cartilage formation disorder or the propensity to develop a bone, connective tissue, or cartilage formation disorder in an organism. This method includes detecting an alteration in the sequence, or a portion of the sequence, of a 954, histone deacetylase, 215, 307, 572, 1116A, 1548, Casein kinase 1α, 299, 994, TCP-1 Beta, Non-Muscle Adenylosuccinate Synthase, Nuclear Cap Binding Protein Subunit 2, Ornithine Decarboxylase, or 1447 nucleic acid molecule in a sample derived from a first organism and comparing the sequence to that of a 954, histone deacetylase, 215, 307, 572, 1116A, 1548, Casein kinase 1α, 299, 994, TCP-1 Beta, Non-Muscle Adenylosuccinate Synthase, Nuclear Cap Binding Protein Subunit 2, Ornithine Decarboxylase, or 1447 nucleic acid molecule derived from a second, control organism, where an alteration of the 954, histone deacetylase, 215, 307, 572, 1116A, 1548, Casein kinase 1α, 299, 994, TCP-1 Beta, Non-Muscle Adenylosuccinate Synthase, Nuclear Cap Binding Protein Subunit 2, Ornithine Decarboxylase, or 1447 sequence in the first organism relative to the second organism is indicative of the first organism having or having a propensity to develop a bone, connective tissue, or cartilage formation disorder. In the alternative, the alteration may be an increase or a decrease in the expression or activity of a nucleic acid molecule including the sequence of SEQ ID NO:9, 31, 33, 35, 37, 39, 41, 67, 69, 123, 133, 135, 137, 143, or 151.

In a further aspect, the invention features a method for identifying a candidate compound for the treatment of a bone, connective tissue, or cartilage formation disorder. This method includes the steps of: (a) contacting a polypeptide including the amino acid sequence of SEQ ID NO:10, 32, 34, 36, 38, 40, 42, 68, 70, or 152 with a candidate compound, and (b) detecting an interaction of the polypeptide with the candidate compound, where a candidate compound that binds the polypeptide may be used to treat a bone, connective tissue, or cartilage formation disorder.

In a further aspect, the invention features a method for diagnosing a cell death disorder or the propensity to develop a cell death disorder in an organism. This method includes detecting an alteration in the level of U2AF, Kinesin Related Motor Protein EG5, 459, 299, 1373, Denticleless, Telomeric Repeat Factor 2, SIL, U1 Small Nuclear Ribonuclear Protein C, Ski Interacting Protein, 297, Small Nuclear Ribonucleoprotein D1, DNA Polymerase Epsilon Subunit B, 1045, Spliceosome Associated Protein 49, DNA Replication Licensing Factor MCM7, 1581, DEAD-Box RNA Helicase (DEAD5 or DEAD19), ISWI/SNF2, XCAP-C, DNA Replication Licensing Factor MCM2, DNA Replication Licensing Factor MCM3, Nuclear Cap Binding Protein Subunit 2, Ornithine Decarboxylase, ARS2, Protein Phosphatase 1 Nuclear Targeting Subunit, Valyl-tRNA Synthase, Translation Elongation Factor eEF1 Alpha, Non-Muscle Adenylosuccinate Synthase, or 821-02 polypeptide expression in a sample derived from a first organism and comparing the level of expression to that of a U2AF, Kinesin Related Motor Protein EG5, 459, 299, 1373, Denticleless, Telomeric Repeat Factor 2, SIL, U1 Small Nuclear Ribonuclear Protein C, Ski Interacting Protein, 297, Small Nuclear Ribonucleoprotein D1, DNA Polymerase Epsilon Subunit B, 1045, Spliceosome Associated Protein 49, DNA Replication Licensing Factor MCM7, 1581, DEAD-Box RNA Helicase (DEAD5 or DEAD19), ISWI/SNF2, XCAP-C, DNA Replication Licensing Factor MCM2, DNA Replication Licensing Factor MCM3, Nuclear Cap Binding Protein Subunit 2, Omithine Decarboxylase, ARS2, Protein Phosphatase 1 Nuclear Targeting Subunit, Valyl-tRNA Synthase, Translation Elongation Factor eEF1 Alpha, Non-Muscle Adenylosuccinate Synthase, or 821-02 polypeptide in a sample derived from a second, control organism, where an alteration in the level of expression or activity of the U2AF, Kinesin Related Motor Protein EG5, 459, 299, 1373, Denticleless, Telomeric Repeat Factor 2, SIL, U1 Small Nuclear Ribonuclear Protein C, Ski Interacting Protein, 297, Small Nuclear Ribonucleoprotein D1, DNA Polymerase Epsilon Subunit B, 1045, Spliceosome Associated Protein 49, DNA Replication Licensing Factor MCM7, 1581, DEAD-Box RNA Helicase (DEAD5 or DEAD19), ISWI/SNF2, XCAP-C, DNA Replication Licensing Factor MCM2, DNA Replication Licensing Factor MCM3, Nuclear Cap Binding Protein Subunit 2, Omithine Decarboxylase, ARS2, Protein Phosphatase 1 Nuclear Targeting Subunit, Valyl-tRNA Synthase, Translation Elongation Factor eEF1 Alpha, Non-Muscle Adenylosuccinate Synthase, or 821-02 polypeptide in the first organism relative to the second organism is indicative of the first organism having or having a propensity to develop a cell death disorder.

In a further aspect, the invention features a method for diagnosing a cell death disorder or the propensity to develop a cell death disorder in an organism. This method includes detecting an alteration in the sequence, or a portion of the sequence, of a U2AF, Kinesin Related Motor Protein EG5, 459, 299, 1373, Denticleless, Telomeric Repeat Factor 2, SIL, U1 Small Nuclear Ribonuclear Protein C, Ski Interacting Protein, 297, Small Nuclear Ribonucleoprotein D1, DNA Polymerase Epsilon Subunit B, 1045, Spliceosome Associated Protein 49, DNA Replication Licensing Factor MCM7, 1581, DEAD-Box RNA Helicase (DEAD5 or DEAD19), ISWI/SNF2, XCAP-C, DNA Replication Licensing Factor MCM2, DNA Replication Licensing Factor MCM3, Nuclear Cap Binding Protein Subunit 2, Ornithine Decarboxylase, ARS2, Protein Phosphatase 1 Nuclear Targeting Subunit, Valyl-tRNA Synthase, Translation Elongation Factor eEF1 Alpha, Non-Muscle Adenylosuccinate Synthase, or 821-02 nucleic acid molecule in a sample derived from a first organism and comparing the sequence to that of a U2AF, Kinesin Related Motor Protein EG5, 459, 299, 1373, Denticleless, Telomeric Repeat Factor 2, SIL, U1 Small Nuclear Ribonuclear Protein C, Ski Interacting Protein, 297, Small Nuclear Ribonucleoprotein D1, DNA Polymerase Epsilon Subunit B, 1045, Spliceosome Associated Protein 49, DNA Replication Licensing Factor MCM7, 1581, DEAD-Box RNA Helicase (DEAD5 or DEAD19), ISWI/SNF2, XCAP-C, DNA Replication Licensing Factor MCM2, DNA Replication Licensing Factor MCM3, Nuclear Cap Binding Protein Subunit 2, Ornithine Decarboxylase, ARS2, Protein Phosphatase 1 Nuclear Targeting Subunit, Valyl-tRNA Synthase, Translation Elongation Factor eEF1 Alpha, Non-Muscle Adenylosuccinate Synthase, or 821-02 nucleic acid molecule derived from a second, control organism, where an alteration of the U2AF, Kinesin Related Motor Protein EG5, 459, 299, 1373, Denticleless, Telomeric Repeat Factor 2, SIL, U1 Small Nuclear Ribonuclear Protein C, Ski Interacting Protein, 297, Small Nuclear Ribonucleoprotein D1, DNA Polymerase Epsilon Subunit B, 1045, Spliceosome Associated Protein 49, DNA Replication Licensing Factor MCM7, 1581, DEAD-Box RNA Helicase (DEAD5 or DEAD19), ISWI/SNF2, XCAP-C, DNA Replication Licensing Factor MCM2, DNA Replication Licensing Factor MCM3, Nuclear Cap Binding Protein Subunit 2, Ornithine Decarboxylase, ARS2, Protein Phosphatase 1 Nuclear Targeting Subunit, Valyl-tRNA Synthase, Translation Elongation Factor eEF1 Alpha, Non-Muscle Adenylosuccinate Synthase, or 821-02 sequence in the first organism relative to the second organism is indicative of the first organism having or having a propensity to develop a cell death disorder. In the alternative, the alteration may be an increase or a decrease in the expression or activity of a nucleic acid molecule including the sequence of SEQ ID NO:7, 57, 59, 67, 71, 73, 79, 81, 83, 85, 87, 91, 93, 95, 97, 101, 103, 105, 107, 111, 113, 115, 117, 119, 127, 133, 135, 137, 139, or 145.

In a further aspect, the invention features a method for identifying a candidate compound for the treatment of a cell death disorder. This method includes the steps of: (a) contacting a polypeptide including the amino acid sequence of SEQ ID NO:8, 58, 60, 68, 72, 74, 80, 82, 84, 86, 88, 92, 94, 98, 102, 104, 106, 108, 112, 114, 116, 118, 120, 128, 134, 136, 138, 140, or 146 with a candidate compound, and (b) detecting an interaction of the polypeptide with the candidate compound, where a candidate compound that binds the polypeptide may be used to treat a cell death disorder.

In a further aspect, the invention features a method for diagnosing a circulatory disorder or the propensity to develop a circulatory disorder in an organism. This method includes detecting an alteration in the level of 904, 1463, 429, or Mitochondrial Inner Membrane Translocating (rTIM23) polypeptide expression in a sample derived from a first organism and comparing the level of expression to that of a 904, 1463, 429, or Mitochondrial Inner Membrane Translocating (rTIM23) polypeptide in a sample derived from a second, control organism, where an alteration in the level of expression or activity of the 904, 1463, 429, or Mitochondrial Inner Membrane Translocating (rTIM23) polypeptide in the first organism relative to the second organism is indicative of the first organism having or having a propensity to develop a circulatory disorder.

In a further aspect, the invention features a method for diagnosing a circulatory disorder or the propensity to develop a circulatory disorder in an organism. This method includes detecting an alteration in the sequence, or a portion of the sequence, of a 904, 1463, 429, or Mitochondrial Inner Membrane Translocating (rTIM23) nucleic acid molecule in a sample derived from a first organism and comparing the sequence to that of a 904, 1463, 429, or Mitochondrial Inner Membrane Translocating (rTIM23) nucleic acid molecule derived from a second, control organism, where an alteration of the 904, 1463, 429, or Mitochondrial Inner Membrane Translocating (rTIM23) sequence in the first organism relative to the second organism is indicative of the first organism having or having a propensity to develop a circulatory disorder. In the alternative, the alteration may be an increase or a decrease in the expression or activity of a nucleic acid molecule including the sequence of SEQ ID NO:1, 47, 141, or 157.

In a further aspect, the invention features a method for identifying a candidate compound for the treatment of a circulatory disorder. This method includes the steps of: (a) contacting a polypeptide including the amino acid sequence of SEQ ID NO:2, 48, 142, or 158 with a candidate compound, and (b) detecting an interaction of the polypeptide with the candidate compound, where a candidate compound that binds the polypeptide may be used to treat a circulatory disorder.

In a further aspect, the invention features a method for diagnosing a craniofacial defect or the propensity to develop a craniofacial defect in an organism. This method includes detecting an alteration in the level of 215, 307, 572, 1116A, 1548, Casein Kinase 1α, 299, 297, DNA Replication Licensing Factor MCM2, 1257, TCP-1 Beta, Nuclear Cap Binding Protein Subunit 2, Non-Muscle Adenylosuccinate Synthase, or Omithine Decarboxylase polypeptide expression in a sample derived from a first organism and comparing the level of expression to that of a 215, 307, 572, 1116A, 1548, Casein Kinase 1α, 299, 297, DNA Replication Licensing Factor MCM2, 1257, TCP-1 Beta, Nuclear Cap Binding Protein Subunit 2, Non-Muscle Adenylosuccinate Synthase, or Ornithine Decarboxylase polypeptide in a sample derived from a second, control organism, where an alteration in the level of expression or activity of the 215, 307, 572, 1116A, 1548, Casein Kinase 1α, 299, 297, DNA Replication Licensing Factor MCM2, 1257, TCP-1 Beta, Nuclear Cap Binding Protein Subunit 2, Non-Muscle Adenylosuccinate Synthase, or Ornithine Decarboxylase polypeptide in the first organism relative to the second organism is indicative of the first organism having or having a propensity to develop a craniofacial defect.

In a further aspect, the invention features a method for diagnosing a craniofacial defect or the propensity to develop a craniofacial defect in an organism. This method includes detecting an alteration in the sequence, or a portion of the sequence, of a 215, 307, 572, 1116A, 1548, Casein Kinase 1α, 299, 297, DNA Replication Licensing Factor MCM2, 1257, TCP-1 Beta, Nuclear Cap Binding Protein Subunit 2, Non-Muscle Adenylosuccinate Synthase, or Ornithine Decarboxylase nucleic acid molecule in a sample derived from a first organism and comparing the sequence to that of a 215, 307, 572, 1116A, 1548, Casein Kinase 1α, 299, 297, DNA Replication Licensing Factor MCM2, 1257, TCP-1 Beta, Nuclear Cap Binding Protein Subunit 2, Non-Muscle Adenylosuccinate Synthase, or Ornithine Decarboxylase nucleic acid molecule derived from a second, control organism, where an alteration of the 215, 307, 572, 1116A, 1548, Casein Kinase 1α, 299, 297, DNA Replication Licensing Factor MCM2, 1257, TCP-1 Beta, Nuclear Cap Binding Protein Subunit 2, Non-Muscle Adenylosuccinate Synthase, or Ornithine Decarboxylase sequence in the first organism relative to the second organism is indicative of the first organism having or having a propensity to develop a craniofacial defect. In the alternative, the alteration may be an increase or a decrease in the expression or activity of a nucleic acid molecule including the sequence of SEQ ID NO:31, 33, 35, 37, 39, 41, 67, 87, 115, 123, 129, 133, 135, or 137.

In a further aspect, the invention features a method for identifying a candidate compound for the treatment of a craniofacial defect. This method includes the steps of: (a) contacting a polypeptide including the amino acid sequence of SEQ ID NO:32, 34, 36, 38, 40, 42, 68, 88, 116, 124, 130, 134, 136, or 138 with a candidate compound, and (b) detecting an interaction of the polypeptide with the candidate compound, where a candidate compound that binds the polypeptide may be used to treat a craniofacial defect.

In a further aspect, the invention features a method for diagnosing a hearing disorder or the propensity to develop a hearing disorder in an organism. This method includes detecting an alteration in the level of U1 Small Nuclear Ribonucleoprotein C, ARS2, TCP-1 Eta, or Histone Deacetylase polypeptide expression in a sample derived from a first organism and comparing the level of expression to that of a U1 Small Nuclear Ribonucleoprotein C, ARS2, TCP-1 Eta, or Histone Deacetylase polypeptide in a sample derived from a second, control organism, where an alteration in the level of expression or activity of the U1 Small Nuclear Ribonucleoprotein C, ARS2, or TCP-1 Eta, Histone Deacetylase polypeptide in the first organism relative to the second organism is indicative of the first organism having or having a propensity to develop a hearing disorder.

In a further aspect, the invention features a method for diagnosing a hearing disorder or the propensity to develop a hearing disorder in an organism. This method includes detecting an alteration in the sequence, or a portion of the sequence, of a U1 Small Nuclear Ribonucleoprotein C, ARS2, TCP-1 Eta, or Histone Deacetylase nucleic acid molecule in a sample derived from a first organism and comparing the sequence to that of a U1 Small Nuclear Ribonucleoprotein C, ARS2, TCP-1 Eta, or Histone Deacetylase nucleic acid molecule derived from a second, control organism, where an alteration of the U1 Small Nuclear Ribonucleoprotein C, ARS2, TCP-1 Eta, or Histone Deacetylase sequence in the first organism relative to the second organism is indicative of the first organism having or having a propensity to develop a hearing disorder. In the alternative, the alteration may be an increase or a decrease in the expression or activity of a nucleic acid molecule including the sequence of SEQ ID NO:83, 125, 145, or 151.

In a further aspect, the invention features a method for identifying a candidate compound for the treatment of a hearing disorder. This method includes the steps of: (a) contacting a polypeptide including the amino acid sequence of SEQ ID NO:84, 125, 146, or 152 with a candidate compound, and (b) detecting an interaction of the polypeptide with the candidate compound, where a candidate compound that binds the polypeptide may be used to treat a hearing disorder.

In a further aspect, the invention features a method for diagnosing diabetes or the propensity to develop diabetes in an organism. This method includes detecting an alteration in the level of 429 or 1447 polypeptide expression in a sample derived from a first organism and comparing the level of expression to that of a 429 or 1447 polypeptide in a sample derived from a second, control organism, where an alteration in the level of expression or activity of the 429 or 1447 polypeptide in the first organism relative to the second organism is indicative of the first organism having or having a propensity to develop diabetes.

In a further aspect, the invention features a method for diagnosing diabetes or the propensity to develop diabetes in an organism. This method includes detecting an alteration in the sequence, or a portion of the sequence, of a 429 or 1447 nucleic acid molecule in a sample derived from a first organism and comparing the sequence to that of a 429 or 1447 nucleic acid molecule derived from a second, control organism, where an alteration of the 429 or 1447 sequence in the first organism relative to the second organism is indicative of the first organism having or having a propensity to develop diabetes. In the alternative, the alteration may be an increase or a decrease in the expression or activity of a nucleic acid molecule including the sequence of SEQ ID NO:47 or 143.

In a further aspect, the invention features a method for identifying a candidate compound for the treatment of diabetes. This method includes the steps of: (a) contacting a polypeptide including the amino acid sequence of SEQ ID NO:48 or 144 with a candidate compound, and (b) detecting an interaction of the polypeptide with the candidate compound, where a candidate compound that binds the polypeptide may be used to treat diabetes.

In a further aspect, the invention features a method for diagnosing heart defect or the propensity to develop a heart defect in an organism. This method includes detecting an alteration in the level of 1548, Histone Deacetylase, Nuclear Cap Binding Protein Subunit 2, Mitochondrial Inner Membrane Translocating Protein (rTIM23), or TCP-1 Alpha polypeptide expression in a sample derived from a first organism and comparing the level of expression to that of a 1548, Histone Deacetylase, Nuclear Cap Binding Protein Subunit 2, Mitochondrial Inner Membrane Translocating Protein (rTIM23), or TCP-1 Alpha polypeptide in a sample derived from a second, control organism, where an alteration in the level of expression or activity of the 1548, Histone Deacetylase, Nuclear Cap Binding Protein Subunit 2, Mitochondrial Inner Membrane Translocating Protein (rTIM23), or TCP-1 Alpha polypeptide in the first organism relative to the second organism is indicative of the first organism having or having a propensity to develop a heart defect.

In a further aspect, the invention features a method for diagnosing heart defect or the propensity to develop heart defect in an organism. This method includes detecting an alteration in the sequence, or a portion of the sequence, of a 1548, Histone Deacetylase, Nuclear Cap Binding Protein Subunit 2, Mitochondrial Inner Membrane Translocating Protein (rTIM23), or TCP-1 Alpha nucleic acid molecule in a sample derived from a first organism and comparing the sequence to that of a 1548, Histone Deacetylase, Nuclear Cap Binding Protein Subunit 2, Mitochondrial Inner Membrane Translocating Protein (rTIM23), or TCP-1 Alpha nucleic acid molecule derived from a second, control organism, where an alteration of the 1548, Histone Deacetylase, Nuclear Cap Binding Protein Subunit 2, Mitochondrial Inner Membrane Translocating Protein (rTIM23), or TCP-1 Alpha sequence in the first organism relative to the second organism is indicative of the first organism having or having a propensity to develop a heart defect. In the alternative, the alteration may be an increase or a decrease in the expression or activity of a nucleic acid molecule including the sequence of SEQ ID NO:39, 77, 135, 141, or 151.

In a further aspect, the invention features a method for identifying a candidate compound for the treatment of a heart defect. This method includes the steps of: (a) contacting a polypeptide including the amino acid sequence of SEQ ID NO:40, 136, or 152 with a candidate compound, and (b) detecting an interaction of the polypeptide with the candidate compound, where a candidate compound that binds the polypeptide may be used to treat a heart defect.

In a further aspect, the invention features a method for diagnosing infertility or the propensity to develop infertility in an organism. This method includes detecting an alteration in the level of Spinster polypeptide expression in a sample derived from a first organism and comparing the level of expression to that of a Spinster polypeptide in a sample derived from a second, control organism, where an alteration in the level of expression or activity of the Spinster polypeptide in the first organism relative to the second organism is indicative of the first organism having or having a propensity to develop infertility.

In a further aspect, the invention features a method for diagnosing infertility or the propensity to develop infertility in an organism. This method includes detecting an alteration in the sequence, or a portion of the sequence, of a Spinster nucleic acid molecule in a sample derived from a first organism and comparing the sequence to that of a Spinster nucleic acid molecule derived from a second, control organism, where an alteration of the Spinster sequence in the first organism relative to the second organism is indicative of the first organism having or having a propensity to develop infertility. In the alternative, the alteration may be an increase or a decrease in the expression or activity of a nucleic acid molecule including the sequence of SEQ ID NO:51.

In a further aspect, the invention features a method for identifying a candidate compound for the treatment of infertility. This method includes the steps of: (a) contacting a polypeptide including the amino acid sequence of SEQ ID NO:52 with a candidate compound, and (b) detecting an interaction of the polypeptide with the candidate compound, where a candidate compound that binds the polypeptide may be used to treat infertility.

In a further aspect, the invention features a method for diagnosing a limb formation defect or the propensity to develop a limb formation defect in an organism. This method includes detecting an alteration in the level of Casein Kinase 1α or Histone Deacetylase polypeptide expression in a sample derived from a first organism and comparing the level of expression to that of a Casein Kinase 1α or Histone Deacetylase polypeptide in a sample derived from a second, control organism, where an alteration in the level of expression or activity of the Casein Kinase 1α or Histone Deacetylase polypeptide in the first organism relative to the second organism is indicative of the first organism having or having a propensity to develop a limb formation defect.

In a further aspect, the invention features a method for diagnosing a limb formation defect or the propensity to develop a limb formation defect in an organism. This method includes detecting an alteration in the sequence, or a portion of the sequence, of a Casein Kinase 1α or Histone Deacetylase nucleic acid molecule in a sample derived from a first organism and comparing the sequence to that of a Casein Kinase 1α or Histone Deacetylase nucleic acid molecule derived from a second, control organism, where an alteration of the Casein Kinase 1α or Histone Deacetylase sequence in the first organism relative to the second organism is indicative of the first organism having or having a propensity to develop a limb formation defect. In the alternative, the alteration may be an increase or a decrease in the expression or activity of a nucleic acid molecule including the sequence of SEQ ID NO:41 or 151.

In a further aspect, the invention features a method for identifying a candidate compound for the treatment of a limb formation defect. This method includes the steps of: (a) contacting a polypeptide including the amino acid sequence of SEQ ID NO:42 or 152 with a candidate compound, and (b) detecting an interaction of the polypeptide with the candidate compound, where a candidate compound that binds the polypeptide may be used to treat a limb formation defect.

In a further aspect, the invention features a method for diagnosing mental retardation or the propensity to develop mental retardation in an organism. This method includes detecting an alteration in the level of U2AF, 1463, VPSP18, 60S Ribosomal Protein L35, 299, 1373, Telomeric Repeat Factor 2, SIL, U1 Small Ribonucleoprotein C, SKIP, 297, Small Nuclear Ribonucleoprotein D1, DNA Polymerase Epsilon Subunit B, 1045, Spliceosome Associated Protein 49, DNA Replication Licensing Factor MCM7, DEAD-Box RNA Helicase (DEAD5 or DEAD19), 1581, ISWI/SNF, Chromosomal Assembly Protein (XCAP-C), DNA Replication Licensing Factor MCM2, DNA Replication Licensing Factor MCM3, Valyl-tRNA Synthase, 40S Ribosomal Protein S5, TCP-1 Alpha, TCP-1 Beta, Translation Elongation Factor eEF1 Alpha, 1257, 60S Ribosomal Protein L24, Non-Muscle Adenylosuccinate Synthase, Nuclear Cap Binding Protein Subunit 2, ARS2, BAF53a, Histone Deacetylase, Protein Phosphatase 1 Nuclear Targeting Subunit protein (PNUTS), Omithine Decarboxylase, 1447, 1262, 994, or TAFII-55 polypeptide expression in a sample derived from a first organism and comparing the level of expression to that of a U2AF, 1463, VPSP18, 60S Ribosomal Protein L35, 299, 1373, Telomeric Repeat Factor 2, SIL, U1 Small Ribonucleoprotein C, SKIP, 297, Small Nuclear Ribonucleoprotein D1, DNA Polymerase Epsilon Subunit B, 1045, Spliceosome Associated Protein 49, DNA Replication Licensing Factor MCM7, DEAD-Box RNA Helicase (DEAD5 or DEAD19), 1581, ISWI/SNF, Chromosomal Assembly Protein (XCAP-C), DNA Replication Licensing Factor MCM2, DNA Replication Licensing Factor MCM3, Valyl-tRNA Synthase, 40S Ribosomal Protein S5, TCP-1 Alpha, TCP-1 Beta, Translation Elongation Factor eEF1 Alpha, 1257, 60S Ribosomal Protein L24, Non-Muscle Adenylosuccinate Synthase, Nuclear Cap Binding Protein Subunit 2, ARS2, BAF53a, Histone Deacetylase, Protein Phosphatase 1 Nuclear Targeting Subunit protein (PNUTS), Omithine Decarboxylase, 1447, 1262, 994, or TAFII-55 polypeptide in a sample derived from a second, control organism, where an alteration in the level of expression or activity of the U2AF, 1463, VPSP18, 60S Ribosomal Protein L35, 299, 1373, Telomeric Repeat Factor 2, SIL, U1 Small Ribonucleoprotein C, SKIP, 297, Small Nuclear Ribonucleoprotein D1, DNA Polymerase Epsilon Subunit B, 1045, Spliceosome Associated Protein 49, DNA Replication Licensing Factor MCM7, DEAD-Box RNA Helicase (DEAD5 or DEAD19), 1581, ISWI/SNF, Chromosomal Assembly Protein (XCAP-C), DNA Replication Licensing Factor MCM2, DNA Replication Licensing Factor MCM3, Valyl-tRNA Synthase, 40S Ribosomal Protein S5, TCP-1 Alpha, TCP-1 Beta, Translation Elongation Factor eEF1 Alpha, 1257, 60S Ribosomal Protein L24, Non-Muscle Adenylosuccinate Synthase, Nuclear Cap Binding Protein Subunit 2, ARS2, BAF53a, Histone Deacetylase, Protein Phosphatase 1 Nuclear Targeting Subunit protein (PNUTS), Omithine Decarboxylase, 1447, 1262, 994, or TAFII-55 polypeptide in the first organism relative to the second organism is indicative of the first organism having or having a propensity to develop mental retardation.

In a further aspect, the invention features a method for diagnosing mental retardation or the propensity to develop mental retardation in an organism. This method includes detecting an alteration in the sequence, or a portion of the sequence, of a U2AF, 1463, VPSP18, 60S Ribosomal Protein L35, 299, 1373, Telomeric Repeat Factor 2, SIL, U1 Small Ribonucleoprotein C, SKIP, 297, Small Nuclear Ribonucleoprotein D1, DNA Polymerase Epsilon Subunit B, 1045, Spliceosome Associated Protein 49, DNA Replication Licensing Factor MCM7, DEAD-Box RNA Helicase (DEAD5 or DEAD19), 1581, ISWI/SNF, Chromosomal Assembly Protein (XCAP-C), DNA Replication Licensing Factor MCM2, DNA Replication Licensing Factor MCM3, Valyl-tRNA Synthase, 40S Ribosomal Protein S5, TCP-1 Alpha, TCP-1 Beta, Translation Elongation Factor eEF1 Alpha, 1257, 60S Ribosomal Protein L24, Non-Muscle Adenylosuccinate Synthase, Nuclear Cap Binding Protein Subunit 2, ARS2, BAF53a, Histone Deacetylase, Protein Phosphatase 1 Nuclear Targeting Subunit protein (PNUTS), Ornithine Decarboxylase, 1447, 1262, 994, or TAFII-55 nucleic acid molecule in a sample derived from a first organism and comparing the sequence to that of a U2AF, 1463, VPSP18, 60S Ribosomal Protein L35, 299, 1373, Telomeric Repeat Factor 2, SIL, U1 Small Ribonucleoprotein C, SKIP, 297, Small Nuclear Ribonucleoprotein D1, DNA Polymerase Epsilon Subunit B, 1045, Spliceosome Associated Protein 49, DNA Replication Licensing Factor MCM7, DEAD-Box RNA Helicase (DEAD5 or DEAD19), 1581, ISWI/SNF, Chromosomal Assembly Protein (XCAP-C), DNA Replication Licensing Factor MCM2, DNA Replication Licensing Factor MCM3, Valyl-tRNA Synthase, 40S Ribosomal Protein S5, TCP-1 Alpha, TCP-1 Beta, Translation Elongation Factor eEF1 Alpha, 1257, 60S Ribosomal Protein L24, Non-Muscle Adenylosuccinate Synthase, Nuclear Cap Binding Protein Subunit 2, ARS2, BAF53a, Histone Deacetylase, Protein Phosphatase 1 Nuclear Targeting Subunit protein (PNUTS), Ornithine Decarboxylase, 1447, 1262, 994, or TAFII-55 nucleic acid molecule derived from a second, control organism, where an alteration of the U2AF, 1463, VPSP18, 60S Ribosomal Protein L35, 299, 1373, Telomeric Repeat Factor 2, SIL, U1 Small Ribonucleoprotein C, SKIP, 297, Small Nuclear Ribonucleoprotein D1, DNA Polymerase Epsilon Subunit B, 1045, Spliceosome Associated Protein 49, DNA Replication Licensing Factor MCM7, DEAD-Box RNA Helicase (DEAD5 or DEAD19), 1581, ISWLI/SNF, Chromosomal Assembly Protein (XCAP-C), DNA Replication Licensing Factor MCM2, DNA Replication Licensing Factor MCM3, Valyl-tRNA Synthase, 40S Ribosomal Protein S5, TCP-1 Alpha, TCP-1 Beta, Translation Elongation Factor eEF1 Alpha, 1257, 60S Ribosomal Protein L24, Non-Muscle Adenylosuccinate Synthase, Nuclear Cap Binding Protein Subunit 2, ARS2, BAF53a, Histone Deacetylase, Protein Phosphatase 1 Nuclear Targeting Subunit protein (PNUTS), Ornithine Decarboxylase, 1447, 1262, 994, or TAFII-55 sequence in the first organism relative to the second organism is indicative of the first organism having or having a propensity to develop mental retardation. In the alternative, the alteration may be an increase or a decrease in the expression or activity of a nucleic acid molecule including the sequence of SEQ ID NO:7, 21, 25, 67, 69, 71, 79, 81, 83, 85, 89, 91, 93, 97, 101, 103, 105, 107, 111, 113, 115, 117, 119, 121, 123, 127, 129, 131, 133, 135, 137, 139, 143, 145, 149, 151, 155, or 157.

In a further aspect, the invention features a method for identifying a candidate compound for the treatment of mental retardation. This method includes the steps of: (a) contacting a polypeptide including the amino acid sequence of SEQ ID NO:8, 20, 22, 26, 68, 70, 72, 80, 82, 84, 86, 90, 92, 94, 98, 102, 104, 106, 108, 112, 114, 116, 118, 120, 122, 124, 128, 130, 132, 134, 136, 138, 140, 144, 146, 150, 152, 156, or 158 with a candidate compound, and (b) detecting an interaction of the polypeptide with the candidate compound, where a candidate compound that binds the polypeptide ma be used to treat mental retardation.

In a further aspect, the invention features a method for diagnosing a muscle defect or the propensity to develop a muscle defect in an organism. This method includes detecting an alteration in the level of 428 or Denticleless polypeptide expression in a sample derived from a first organism and comparing the level of expression to that of a 428 or Denticleless polypeptide in a sample derived from a second, control organism, where an alteration in the level of expression or activity of the 428 or Denticleless polypeptide in the first organism relative to the second organism is indicative of the first organism having or having a propensity to develop a muscle defect.

In a further aspect, the invention features a method for diagnosing a muscle defect or the propensity to develop a muscle defect in an organism. This method includes detecting an alteration in the sequence, or a portion of the sequence, of a 428 or Denticleless nucleic acid molecule in a sample derived from a first organism and comparing the sequence to that of a 428 or Denticleless nucleic acid molecule derived from a second, control organism, where an alteration of the 428 or Denticleless sequence in the first organism relative to the second organism is indicative of the first organism having or having a propensity to develop a muscle defect. In the alternative, the alteration may be an increase or a decrease in the expression or activity of a nucleic acid molecule including the sequence of SEQ ID NO:49 or 73.

In a further aspect, the invention features a method for identifying a candidate compound for the treatment of a muscle defect. This method includes the steps of: (a) contacting a polypeptide including the amino acid sequence of SEQ ID NO:50 or 74 with a candidate compound, and (b) detecting an interaction of the polypeptide with the candidate compound, where a candidate compound that binds the polypeptide may be used to treat a muscle defect.

In a further aspect, the invention features a method for diagnosing a neurodegenerative disorder or the propensity to develop a neurodegenerative disorder in an organism. This method includes detecting an alteration in the level of Vesicular Integral Membrane Protein VIP 36, 297, 40S Ribosomal Protein S5, V-ATPase 16 kDa Proteolytic Subunit, Kinesin Related Motor Protein EG5, 299, 821-02, Valyl-tRNA Synthase, or Translation Elongation Factor eEF1 Alpha polypeptide expression in a sample derived from a first organism and comparing the level of expression to that of a Vesicular Integral Membrane Protein VIP 36, 297, 40S Ribosomal Protein S5, V-ATPase 16 kDa Proteolytic Subunit, Kinesin Related Motor Protein EG5, 299, 821-02, Valyl-tRNA Synthase, or Translation Elongation Factor eEF1 Alpha polypeptide in a sample derived from a second, control organism, where an alteration in the level of expression or activity of the Vesicular Integral Membrane Protein VIP 36, 297, 40S Ribosomal Protein S5, V-ATPase 16 kDa Proteolytic Subunit, Kinesin Related Motor Protein EG5, 299, 821-02, Valyl-tRNA Synthase, or Translation Elongation Factor eEF1 Alpha polypeptide in the first organism relative to the second organism is indicative of the first organism having or having a propensity to develop a neurodegenerative disorder.

In a further aspect, the invention features a method for diagnosing a neurodegenerative disorder or the propensity to develop a neurodegenerative disorder in an organism. This method includes detecting an alteration in the sequence of a Vesicular Integral Membrane Protein VIP 36, 297, 40S Ribosomal Protein S5, V-ATPase 16 kDa Proteolytic Subunit, Kinesin Related Motor Protein EG5, 299, 821-02, Valyl-tRNA Synthase, or Translation Elongation Factor eEF1 Alpha nucleic acid molecule in a sample derived from a first organism and comparing the sequence to that of a Vesicular Integral Membrane Protein VIP 36,297, 40SRibosomal Protein S5, V-ATPase 16 kDa Proteolytic Subunit, Kinesin Related Motor Protein EG5, 299, 821-02, Valyl-tRNA Synthase, or Translation Elongation Factor eEF1 Alpha nucleic acid molecule derived from a second, control organism, where an alteration of the Vesicular Integral Membrane Protein VIP 36, 297, 40S Ribosomal Protein S5, V-ATPase 16 kDa Proteolytic Subunit, Kinesin Related Motor Protein EG5, 299, 821-02, Valyl-tRNA Synthase, or Translation Elongation Factor eEF1 Alpha sequence in the first organism relative to the second organism is indicative of the first organism having or having a propensity to develop a neurodegenerative disorder. In the alternative, the alteration may be an increase or a decrease in the expression or activity of a nucleic acid molecule including the sequence of SEQ ID NO:19, 57, 65, 67, 87, 95, 119, 121, or 127.

In a further aspect, the invention features a method for identifying a candidate compound for the treatment of a neurodegenerative disorder. This method includes the steps of: (a) contacting a polypeptide including the amino acid sequence of SEQ ID NO:20, 58, 66, 68, 88, 96, 120, 122, or 128 with a candidate compound, and (b) detecting an interaction of the polypeptide with the candidate compound, where a candidate compound that binds the polypeptide may be used to treat a neurodegenerative disorder.

In a further aspect, the invention features a method for diagnosing stroke or the propensity to develop stroke in an organism. This method includes detecting an alteration in the level of 1463 polypeptide expression in a sample derived from a first organism and comparing the level of expression to that of a 1463 polypeptide in a sample derived from a second, control organism, where an alteration in the level of expression or activity of the 1463 polypeptide in the first organism relative to the second organism is indicative of the first organism having or having a propensity to develop stroke.

In a further aspect, the invention features a method for diagnosing stroke or the propensity to develop stroke in an organism. This method includes detecting an alteration in the sequence, or a portion of the sequence of a 1463 nucleic acid molecule in a sample derived from a first organism and comparing the sequence to that of a 1463 nucleic acid molecule derived from a second, control organism, where an alteration of the 1463 sequence in the first organism relative to the second organism is indicative of the first organism having or having a propensity to develop stroke. In the alternative, the alteration may be an increase or a decrease in the expression or activity of a nucleic acid molecule including the sequence of SEQ ID NO:157.

In a further aspect, the invention features a method for identifying a candidate compound for the treatment of stroke. This method includes the steps of: (a) contacting a polypeptide including the amino acid sequence of SEQ ID NO:158 with a candidate compound, and (b) detecting an interaction of the polypeptide with the candidate compound, where a candidate compound that binds the polypeptide may be used to treat stroke.

In a further aspect, the invention features a method for diagnosing a stem cell regeneration disorder or the propensity to develop a stem cell regeneration disorder in an organism. This method includes detecting an alteration in the level of 1447 polypeptide expression in a sample derived from a first organism and comparing the level of expression to that of a 1447 polypeptide in a sample derived from a second, control organism, where an alteration in the level of expression or activity of the 1447 polypeptide in the first organism relative to the second organism is indicative of the first organism having or having a propensity to develop a stem cell regeneration disorder.

In a further aspect, the invention features a method for diagnosing a stem cell regeneration disorder or the propensity to develop a stem cell regeneration disorder in an organism. This method includes detecting an alteration in the sequence, or a portion of the sequence of a 1447 nucleic acid molecule in a sample derived from a first organism and comparing the sequence to that of a 1447 nucleic acid molecule derived from a second, control organism, where an alteration of the 1447 sequence in the first organism relative to the second organism is indicative of the first organism having or having a propensity to develop a stem cell regeneration disorder. In the alternative, the alteration may be an increase or a decrease in the expression or activity of a nucleic acid molecule including the sequence of SEQ ID NO:143.

In a further aspect, the invention features a method for identifying a candidate compound for the treatment of a stem cell regeneration disorder. This method includes the steps of: (a) contacting a polypeptide including the amino acid sequence of SEQ ID NO: 144 with a candidate compound, and (b) detecting an interaction of the polypeptide with the candidate compound, where a candidate compound that binds the polypeptide may be used to treat a stem cell regeneration disorder.

In a further aspect, the invention features a method for diagnosing a visual defect or the propensity to develop a visual defect in an organism. This method includes detecting an alteration in the level of V-ATPase SFD subunit, V-ATPase 16 kDa Proteolytic Subunit, V-ATPase Alpha Subunit, 1463, VPSP18, or 297 polypeptide expression in a sample derived from a first organism and comparing the level of expression to that of a V-ATPase SFD subunit, V-ATPase 16 kDa Proteolytic Subunit, V-ATPase Alpha Subunit, 1463, VPSP18, Ribonucleotide Reductase R1 Class 1, Telomeric Repeat Factor 2, SIL, ISWI/SNF2 or 297 polypeptide in a sample derived from a second, control organism, where an alteration in the level of expression or activity of the V-ATPase SFD subunit, V-ATPase 16 kDa Proteolytic Subunit, V-ATPase Alpha Subunit, 1463, VPSP18, Ribonucleotide Reductase R1 Class 1, Telomeric Repeat Factor 2, SIL, ISWI/SNF2 or 297 polypeptide in the first organism relative to the second organism is indicative of the first organism having or having a propensity to develop a visual defect.

In a further aspect, the invention features a method for diagnosing a visual defect or the propensity to develop a visual defect in an organism. This method includes detecting an alteration in the sequence, or a portion of the sequence of a V-ATPase SFD subunit, V-ATPase 16 kDa Proteolytic Subunit, V-ATPase Alpha Subunit, 1463, VPSP18, Ribonucleotide Reductase R1 Class 1, Telomeric Repeat Factor 2, SIL, ISWI/SNF2 or 297 nucleic acid molecule in a sample derived from a first organism and comparing the sequence to that of a V-ATPase SFD subunit, V-ATPase 16 kDa Proteolytic Subunit, V-ATPase Alpha Subunit, 1463, VPSP18, Ribonucleotide Reductase R1 Class 1, Telomeric Repeat Factor 2, SIL, ISWI/SNF2 or 297 nucleic acid molecule derived from a second, control organism, where an alteration of the V-ATPase SFD subunit, V-ATPase 16 kDa Proteolytic Subunit, V-ATPase Alpha Subunit, 1463, VPSP18, Ribonucleotide Reductase R1 Class 1, Telomeric Repeat Factor 2, SIL, ISWI/SNF2 or 297 sequence in the first organism relative to the second organism is indicative of the first organism having or having a propensity to develop a visual defect. In the alternative, the alteration may be an increase or a decrease in the expression or activity of a nucleic acid molecule including the sequence of SEQ ID NO:15, 17, 19, 21, 55, 79, 81, 87, 111, or 157.

In a further aspect, the invention features a method for identifying a candidate compound for the treatment of a visual defect. This method includes the steps of: (a) contacting a polypeptide including the amino acid sequence of SEQ ID NO:16, 18, 20, 22, 56, 80, 82, 88, 112, or 158 with a candidate compound, and (b) detecting an interaction of the polypeptide with the candidate compound, where a candidate compound that binds the polypeptide may be used to treat a visual defect.

In a further aspect, the invention features a method for diagnosing a pulmonary disorder or the propensity to develop a pulmonary disorder in an organism. This method includes detecting an alteration in the level of Fibroblast Isoform of the ADP/ATP Carrier Protein or TAFII-55 polypeptide expression in a sample derived from a first organism and comparing the level of expression to that of a Fibroblast Isoform of the ADP/ATP Carrier Protein or TAFII-55 polypeptide in a sample derived from a second, control organism, where an alteration in the level of expression or activity of the Fibroblast Isoform of the ADP/ATP Carrier Protein or TAFII-55 polypeptide in the first organism relative to the second organism is indicative of the first organism having or having a propensity to develop a pulmonary disorder.

In a further aspect, the invention features a method for diagnosing a pulmonary disorder or the propensity to develop a pulmonary disorder in an organism. This method includes detecting an alteration in the sequence, or a portion of the sequence, of a Fibroblast Isoform of the ADP/ATP Carrier Protein or TAFII-55 nucleic acid molecule in a sample derived from a first organism and comparing the sequence to that of a Fibroblast Isoform of the ADP/ATP Carrier Protein or TAFII-55 nucleic acid molecule derived from a second, control organism, where an alteration of the Fibroblast Isoform of the ADP/ATP Carrier Protein or TAFII-55 sequence in the first organism relative to the second organism is indicative of the first organism having or having a propensity to develop a pulmonary disorder. In the alternative, the alteration may be an increase or a decrease in the expression or activity of a nucleic acid molecule including the sequence of SEQ ID NO:153 or 155.

In a further aspect, the invention features a method for identifying a candidate compound for the treatment of a pulmonary disorder. This method includes the steps of: (a) contacting a polypeptide including the amino acid sequence of SEQ ID NO:154 or 156 with a candidate compound, and (b) detecting an interaction of the polypeptide with the candidate compound, where a candidate compound that binds the polypeptide may be used to treat a pulmonary disorder.

In a further aspect, the invention features a method for diagnosing a movement disorder or the propensity to develop a movement disorder in an organism. This method includes detecting an alteration in the level of 40S Ribosomal Protein S5 polypeptide expression in a sample derived from a first organism and comparing the level of expression to that of a 40S Ribosomal Protein S5 polypeptide in a sample derived from a second, control organism, where an alteration in the level of expression or activity of the 40S Ribosomal Protein S5 polypeptide in the first organism relative to the second organism is indicative of the first organism having or having a propensity to develop a movement disorder.

In a further aspect, the invention features a method for diagnosing a movement disorder or the propensity to develop a movement disorder in an organism. This method includes detecting an alteration in the sequence, or a portion of the sequence, of a 40S Ribosomal Protein S5 nucleic acid molecule in a sample derived from a first organism and comparing the sequence to that of a 40S Ribosomal Protein S5 nucleic acid molecule derived from a second, control organism, where an alteration of the 40S Ribosomal Protein SS sequence in the first organism relative to the second organism is indicative of the first organism having or having a propensity to develop a movement disorder. In the alternative, the alteration may be an increase or a decrease in the expression or activity of a nucleic acid molecule including the sequence of SEQ ID NO:121.

In a further aspect, the invention features a method for identifying a candidate compound for the treatment of a movement disorder. This method includes the steps of: (a) contacting a polypeptide including the amino acid sequence of SEQ ID NO:122 with a candidate compound, and (b) detecting an interaction of the polypeptide with the candidate compound, where a candidate compound that binds the polypeptide may be used to treat a movement disorder.

In a further aspect, the invention features a method for diagnosing a somite formation disorder or the propensity to develop a somite formation disorder in an organism. This method includes detecting an alteration in the level of 428 or Denticleless polypeptide expression in a sample derived from a first organism and comparing the level of expression to that of a 428 or Denticleless polypeptide in a sample derived from a second, control organism, where an alteration in the level of expression or activity of the 428 or Denticleless polypeptide in the first organism relative to the second organism is indicative of the first organism having or having a propensity to develop a somite formation disorder.

In a further aspect, the invention features a method for diagnosing a somite formation disorder or the propensity to develop a somite formation disorder in an organism. This method includes detecting an alteration in the sequence, or a portion of the sequence, of a 428 or Denticleless nucleic acid molecule in a sample derived from a first organism and comparing the sequence to that of a 428 or Denticleless nucleic acid molecule derived from a second, control organism, where an alteration of the 428 or Denticleless sequence in the first organism relative to the second organism is indicative of the first organism having or having a propensity to develop a somite formation disorder. In the alternative, the alteration may be an increase or a decrease in the expression or activity of a nucleic acid molecule including the sequence of SEQ ID NO:49 or 73.

In a further aspect, the invention features a method for identifying a candidate compound for the treatment of a somite formation disorder. This method includes the steps of: (a) contacting a polypeptide including the amino acid sequence of SEQ ID NO:50 or 74 with a candidate compound, and (b) detecting an interaction of the polypeptide with the candidate compound, where a candidate compound that binds the polypeptide may be used to treat a somite formation disorder.

In a further aspect, the invention features an antibody that specifically binds to a polypeptide described herein and in another aspect, the invention features a zebrafish that includes a mutant nucleic acid molecule described herein.

Generally, the novel nucleic acid and amino acid sequences described herein may be, for example, naturally-occurring. These nucleic acid and amino acid sequences may be, for example, used in protein-protein interaction assays (e.g., two-hybrid, three-hybrid, and co-immunoprecipitation). In addition, the novel nucleic acid sequences described herein may be used to generate transgenic animals, for example, zebrafish, mice, and rats. Furthermore, such transgenic animals may be used in whole animal assays, such as assays to identify candidate compounds potentially useful for treating a disease or disorder. Moreover, these novel nucleic acid and amino acid molecules may be used, for instance, to generate probes and primers, as well as anti-sense nucleic acid sequences complementary to a novel nucleic acid sequence described herein that may be used to inhibit the biological activity of the nucleic acid and amino acid sequences described herein, regardless of the length of the anti-sense nucleic acid sequence. These antisense nucleic acid sequences may be used to treat disease and may also be used to form pharmaceutical compositions.

Definitions

By a “459 protein,” or a “459 polypeptide” is meant a polypeptide that has at least 72% amino acid sequence identity to the zebrafish 459 amino acid sequence of SEQ ID NO:60 over a region spanning at least 233 contiguous amino acids. Desirably, a “459 protein” or a “459 polypeptide” is at least 80%, 85%, 90%, 95%, 97%, 99%, or even 100% identical to the sequence of SEQ ID NO:60 over at least 150, 175, 200, or 233 contiguous amino acids. Polypeptides encoded by splice variants of 459 nucleic acid sequences, as well as by 459 nucleic acids containing a mutation, for example, a mutation resulting from the insertion of a virus at nucleotide 210 of a 459 nucleic acid sequence (e.g., SEQ ID NO:59) are also included in this definition. A “459 protein” or a “459 polypeptide,” as referred to herein, plays a role in kidney development and in cell death during development. In zebrafish, the loss of, or an alteration in, a 459 polypeptide in a cell may result in the development of a cyst in a kidney, a bent body shape, or in the appearance of apoptotic cells in the Central Nervous System (CNS). Accordingly, a “459 protein” or a “459 polypeptide” may be used as a marker for, or to prevent or treat, for example, a kidney disorder, for example, polycystic kidney disease, multicystic kidney disease, or malformation of the kidney; a neurodegenerative disease characterized by excess cell death, for example, Alzheimer's disease, Parkinson's disease, or spinocerebellar ataxia; or may be used for the treatment of a proliferative disorder, such as cancer.

By a “459 nucleic acid sequence” is meant a nucleic acid sequence that has at least 74%, 80%, 85%, 90%, 95%, or 98% identity to the zebrafish 459 nucleic acid sequence of SEQ ID NO:59 over at least 250, 584, 700, 800, 900, 1000, 1500, 2000, or 2500 contiguous nucleotides. In a desirable embodiment, a “459 nucleic acid sequence” is identical to the sequence of SEQ ID NO:59. Nucleic acid molecules consisting of splice variants of 459 nucleic acid sequences, as well as 459 nucleic acid sequences containing a mutation, for example, a mutation resulting from the insertion of a virus, e.g., an insertion at nucleotide 210 of SEQ ID NO:59, are also included in this definition. By a “459 nucleic acid sequence” is also meant a nucleic acid molecule, such as a genomic DNA, cDNA, or RNA (e.g., mRNA) molecule, that encodes a 459 polypeptide or a zebrafish 459 polypeptide (e.g., SEQ ID NO:60), or a fragment thereof, as defined above. A mutation in a 459 nucleic acid molecule can be characterized, for example, by the insertion of a retrovirus into the nucleic acid sequence, for example, using methods described herein. In addition, the invention includes mutations that result in aberrant 459 expression or function, including, as examples, null mutations and mutations causing truncations.

In zebrafish, an alteration in a 459 nucleic acid sequence in a cell may result in the development of a cyst in a kidney, a bent body shape, or in the appearance of apoptotic cells in the Central Nervous System (CNS). Accordingly, a “459 nucleic acid sequence” may be used as a marker for, or to prevent or treat, for example, a kidney disorder, for example, polycystic kidney disease, multicystic kidney disease, or malformation or abnormal development of the kidney; a neurodegenerative disease characterized by excess cell death, for example, Alzheimer's disease, Parkinson's disease, or spinocerebellar ataxia; or may be used for the treatment of a proliferative disorder, such as cancer.

By a “904 protein” or a “904 polypeptide” is meant a polypeptide that has at least 92%, 95%, 97%, or 99% amino acid sequence identity over at least 75, 100, or 150 contiguous amino acids to the zebrafish 904 polypeptide sequence of SEQ ID NO:2. Desirably, a “904 protein” or a “904 polypeptide” is at least 92%, 95%, 97%, or even 100% identical to the sequence of SEQ ID NO:2. Polypeptides encoded by splice variants of 904 nucleic acid sequences, as well as by 904 nucleic acids containing a mutation, for example, a mutation resulting from the insertion of a virus, e.g., an insertion at nucleotide 1315 of SEQ ID NO:1, are also included in this definition. A “904 protein” or a “904 polypeptide,” as referred to herein, plays a role in brain development, compartmentalization, or function. The loss of a 904 polypeptide in a cell may result in an overgrowth of neural tissue, increased vascularization, and in brain hemorrhages. Accordingly, a “904 protein” or a “904 polypeptide” may be used as a marker for, or to prevent or treat, for example, a proliferative disease, such as cancer or neuroblastoma, or a circulatory disorder, such as stroke.

By a “POU2 protein” or a “POU2 polypeptide” is meant a polypeptide that has at least 37%, 40%, 45%, 50%, 60%, 75%, 90%, 95%, or 99% amino acid sequence identity to the zebrafish POU2 polypeptide sequence of SEQ ID NO:4 over a region spanning at least 100, 200, 300, 350 contiguous amino acids. In one desirable embodiment, a “POU2 protein” or a “POU2 polypeptide” is identical to the sequnce of SEQ ID NO:4. Polypeptides encoded by splice variants of POU2 nucleic acid sequences, as well as by POU2 nucleic acid sequences containing a mutation, for example, a mutation resulting from the insertion of a virus, e.g., an insertion at nucleotide 653 or 1088 of SEQ ID NO:3, are also included in this definition. A “POU2 protein” or a “POU2 polypeptide,” as referred to herein, plays a role in neural development, e.g., mid-brain and hind-brain development, as well as, e.g., in zebrafish, in the development of the otolith, or the hair cells of the otolith. Accordingly, a “POU2 protein” or a “POU2” polypeptide” may be used as a marker for, or to prevent or treat, congenital hearing or a sensory disorder, such as Usher syndrome or Waardenburg syndrome, or a congenital brain defect that causes mental retardation, such as Down's syndrome, Chiari Malformation, colpocephaly, holoprosencephaly, lissencephaly, or megalencephaly.

By a “40S Ribosomal Protein S18protein” or a “40S Ribosomal Protein S18 polypeptide” is meant a polypeptide that has at least 97%, 98%, or at least 99% amino acid sequence identity to the zebrafish 40S ribosomal protein S118 sequence of SEQ ID NO:6 over a region spanning at least 75, 100, 115, or 152 contiguous amino acids. Desirably, a “40S Ribosomal Protein S18 protein” or “40S Ribosomal Protein S18 polypeptide” is meant a polypeptide that is identical to the sequence of SEQ ID NO:6. Polypeptides encoded by splice variants of 40S Ribosomal Protein S18 nucleic acid sequences, as well as by 40S Ribosomal Protein S18 nucleic acids containing a mutation, for example, a mutation resulting from the insertion of a virus, e.g., an insertion between nucleotides 220 and 221 of SEQ ID NO:5, are also included in this definition. A. “40S Ribosomal Protein S18 protein” or a “40S Ribosomal Protein S18 polypeptide,” as referred to herein, plays a role in cell proliferation, e.g., neuronal proliferation. For example, in zebrafish, a “40S Ribosomal Protein S18 protein” or a “40S Ribosomal Protein S18 polypeptide,” as referred to herein, plays a role in brain development, neural organization, or compartmentalization, or eye development. In addition, the loss of a zebrafish 40S Ribosomal Protein S18 polypeptide in a cell may result in a kinked tail, a reduced forebrain, and a bigger hind-brain. Accordingly, a “40S Ribosomal Protein S18 protein” or a “40S Ribosomal Protein S18 polypeptide” may be used as a marker for, or to prevent or treat, for example, a proliferative disorder such as cancer or neuroblastoma, or an eye malformation syndrome, such as oculorenal syndrome or Reiger syndrome, or a congenital brain defect that causes mental retardation, such as microencephaly, anencephaly, Down's syndrome, Chiari Malformation, Colpocephaly, holoprosencephaly, lissencephaly, or megalencephaly.

By a U2AF protein” or a “U2AF polypeptide” is meant a polypeptide that has at least 92%, 95%, 97%, or 99% amino acid sequence identity to the zebrafish splicing factor U2AF amino acid sequence of SEQ ID NO:8 over a region spanning at least 150, 175, 200, 225, or 250 contiguous amino acids. Desirably, a “U2AF protein” or a “U2AF polypeptide” is identical to the sequence of SEQ ID NO:8. Polypeptides encoded by splice variants of U2AF nucleic acid sequences, as well as U2AF nucleic acid sequences containing a mutation, for example, a mutation resulting from the insertion of a virus, e.g., an insertion between nucleotides 46 and 47 of the sequence of SEQ ID NO:7, are also included in this definition. A “U2AF protein” or a “U2AF polypeptide,” as referred to herein, plays a role in brain development, particularly in the tectum. The loss of a U2AF polypeptide in a cell may result in brain necrosis, particularly in the tectum. Accordingly, a “U2AF protein,” or a “U2AF polypeptide” may be used as a marker for, or to prevent or treat, for example, a necrotizing brain disorder, such as Leigh's disease, or subacute necrotizing encephalomyelopathy.

By a “954 protein” or a “954 polypeptide” is meant a polypeptide that has at least 93%, 95%, 97%, or 99% amino acid sequence identity to the zebrafish 954 amino acid sequence of SEQ ID NO:10 or SEQ ID NO:159 over a region spanning at least 200, 300, or 334 amino acids. Desirably, a “954 protein” or a “954 polypeptide” is identical to the sequence of SEQ ID NO:10 or SEQ ID NO:159. Polypeptides encoded by splice variants of 954 nucleic acid sequences, as well as 954 nucleic acid sequences containing a mutation, for example, a mutation resulting from the insertion of a virus, e.g., an insertion at nucleotide 432 or 506 of the sequence of SEQ ID NO:9, are also included in this definition. A “954 protein” or a “954 polypeptide,” as referred to herein, plays a role in cartilage development. Accordingly, a “954 protein,” or a “954 polypeptide” may be used as a marker for, or to prevent or treat, for example, a connective tissue disorder such as arthritis.

By a “Neurogenin Related Protein-1,” a “Nrp-1 protein,” a “Nrp-1 polypeptide” or a “Neurogenin Related Protein-1 polypeptide” is meant a polypeptide that is identical to a zebrafish Nrp-1 amino acid sequence, for example, the sequence of SEQ ID NO:12. Polypeptides encoded by splice variants of Nrp-1 nucleic acid sequences, as well as Nrp-1 nucleic acid sequences containing a mutation, for example, a mutation resulting from the insertion of a virus, e.g., an insertion at nucleotide 1149 of the sequence of SEQ ID NO:11, are also included in this definition. A “Neurogenin Related Protein-1,” a “Nrp-1 protein,” or a “Neurogenin Related Protein-1 polypeptide,” as referred to herein, plays a role in cell fate determination or in jaw development. The loss of a Nrp-1 polypeptide in a cell may also result in touch insensitivity in the head, a gaping jaw, and motility problems. Accordingly, a “Neurogenin Related Protein-1,” a “Nrp-1 protein,” a “Nrp-1 polypeptide” or a “Neurogenin Related Protein-1 polypeptide” may be used as a marker for, or to prevent or treat, for example, a movement disorder such as Angelman's syndrome, tardive dyskinesia, spasticity, ataxias, or spinocerebellar ataxia; also neurodegenerative disorders, for example, Huntington's disease, or Parkinson's disease, Alzheimer's disease, or a craniofacial defect, such as Apert, Crouzon, Pfeiffer and Saethre-Chotzen Syndromes

By a “Caudal Protein,” a “Cad-1 protein,” or a “Caudal polypeptide” is meant a polypeptide that is identical to a zebrafish Cad-1 amino acid sequence, for example, the sequence of SEQ ID NO:14. Polypeptides encoded by splice variants of Cad-1 nucleic acid sequences, as well as Cad-1 nucleic acid sequences containing a mutation, for example, a mutation resulting from the insertion of a virus, e.g., an insertion between nucleotides 583 and 584 of the sequence of SEQ ID NO:13, are also included in this definition. A “Caudal Protein,” a “Cad-1 protein,” or a “Caudal polypeptide,” as referred to herein, plays a role in trunk and tail development, motility, touch sensitivity and, e.g., in zebrafish, in the formation of the yolk sac extension. Accordingly, a “Caudal Protein,” a “Cad-1 protein,” or a “Caudal polypeptide” may be used as a marker for, or to prevent or treat, for example, a movement disorder such as Angelman's syndrome, tardive dyskinesia, spasticity, ataxias, or spinocerebellar ataxia, or a neurodegenerative disorder, such as Parkinson's disease, Huntington's disease, amyotrophic lateral sclerosis, or spinocerebellar ataxia.

By a “V-ATPase Alpha Subunit Protein” or a “V-ATPase Alpha Subunit polypeptide” is meant a polypeptide that has at least 77%, 80%, 85%, 90%, 95%, 97%, or 99% identity to the sequence of SEQ ID NO:16, over at least 150, 175, 200, or 226 amino acids. Desirably, a “V-ATPase Alpha Subunit Protein” or a “V-ATPase Alpha Subunit polypeptide” is identical to the sequence of SEQ ID NO:16. Polypeptides encoded by splice variants of V-ATPase Alpha Subunit nucleic acid sequences (e.g., SEQ ID NO:15), as well as V-ATPase Alpha Subunit nucleic acid sequences containing a mutation, for example, a mutation resulting from the insertion of a virus, e.g., an insertion at nucleotide 169 of the sequence of SEQ ID NO:15, are also included in this definition. A “V-ATPase Alpha Subunit polypeptide,” as referred to herein, plays a role in pigmentation in the body or the eye. Accordingly, a “V-ATPase Subunit Alpha polypeptide” or a “V-ATPase Alpha Subunit protein” may be used as a marker for, or to prevent or treat, for example, a variety of disorders, particularly disorders related to pigmentation, such as human pigmentary glaucoma, Wagner's disease, Cockayne's syndrome, Kearns-Sayre disease, Waardenburg syndrome, Usher's syndrorme, or Multiple lentigines syndrome.

By a “V-ATPase SFD Subunit protein” or a “V-ATPase SFD Subunit polypeptide” is meant a polypeptide that has at least 89%, 91%, 93%, 95%, 97%, or 99% identity to the zebrafish V-ATPase subunit SFD sequence of SEQ ID NO:18 over at least 100, 150, 200, 250, 300, 400, or 450 contiguous amino acids. Desirably, a “V-ATPase SFD Subunit protein” or a “V-ATPase SFD Subunit polypeptide” is identical to the sequence of SEQ ID NO:18. Polypeptides encoded by splice variants of V-ATPase SFD Subunit nucleic acid sequences, as well as V-ATPase SFD Subunit nucleic acid sequences containing a mutation, for example, a mutation resulting from the insertion of a virus, e.g., an insertion at nucleotide 31-32 of the sequence of SEQ ID NO:17, are also included in this definition. A “V-ATPase SFD Subunit Protein,” a “V-ATP synthase subunit SFD protein,” or a “V-ATPase SFD Subunit polypeptide,” as referred to herein, plays a role in eye and body pigmentation. Accordingly, a “V-ATPase SFD Subunit protein,” or a “V-ATPase SFD Subunit polypeptide” may be used as a marker for, or to prevent or treat, a variety of disorders, such as developmental disorders, particularly disorders related to pigmentation, for example, human pigmentary glaucoma, Wagner's disease, Cockayne's syndrome, Kearns-Sayre disease, Waardenburg syndrome, Usher's syndrome, or Multiple lentigines syndrome.

By a “V-ATPase 16 kDa Proteolytic Subunit protein” or a “V-ATPase 16 kDa Proteolytic Subunit polypeptide” is meant a polypeptide that has at least 91%, 93%, 95%, or 99% identity to the zebrafish V-ATPase 16 kDa Proteolytic Subunit amino acid sequence of SEQ ID NO:20 over at least 50, 75, 100, 125, or 150 contiguous amino acids. Desirably, a “V-ATPase 16 kDa Proteolytic Subunit protein” or a “V-ATPase 16 kDa Proteolytic Subunit polypeptide” is idenical to the sequence of SEQ ID NO:20. Polypeptides encoded by splice variants of VATPase 16 kDa Proteolytic Subunit nucleic acid sequences, as well as V-ATPase 16 kDa Proteolytic Subunit nucleic acid sequences containing a mutation, for example, a mutation resulting from the insertion of a virus, e.g., an insertion at nucleotide 242-243 of the sequence of SEQ ID NO:19, are also included in this definition. The V-ATPase 16 kDa Proteolytic Subunit nucleic acid sequence plays a role in body and eye pigmentation, and also touch sensitivity. Accordingly, a “V-ATPase 16 kDa Proteolytic Subunit protein,” or a “V-ATPase 16 kDa Proteolytic Subunit polypeptide” may be used as a marker for, or to prevent or treat, for example, a disorder related to pigmentation, such as human pigmentary glaucoma, Wagner's disease, Cockayne's syndrome, Kearns-Sayre disease, Waardenburg syndrome, Usher's syndrome, or Multiple lentigines syndrome, or a neurodegenerative disease, such as Alzheimer's disease, Huntington's disease, Parkinson's disease, or spinocerebellar ataxia.

By a “1463 protein” or a “1463 polypeptide” is meant a polypeptide that has at least 44%, 50%, 60%, 70%, 80%, 90%, or 95% identity to the zebrafish 1463 polypeptide sequence of SEQ ID NO:158 over at least 100, 150, 200, 250, 300, 350, 400, 450, or 500 contiguous amino acids. Desirably, a “1463 protein” or a “1463 polypeptide” is identical to the sequence of SEQ ID NO:158. Polypeptides encoded by splice variants of 1463 nucleic acid sequences, as well as 1463 nucleic acid sequences containing a mutation, for example, a mutation resulting from the insertion of a virus, e.g., an insertion between nucleotides 389 and 390 of the nucleic acid sequence of SEQ ID NO:157 as referred to herein, are also included in this definition. 1463 plays a role in body pigmentation, brain morphogenesis, or angiogenesis. For example, in zebrafish, the loss of a 1463 polypeptide in a cell results in brain dysmorphia, a shortened hind-brain and swollen tectum, or a defect in body pigmentation. Accordingly, a “1463 protein,” or a “1463 polypeptide” may be used as a marker for, or to prevent or treat, for example, disorders related to pigmentation, such as human pigmentary glaucoma, Wagner's disease, Cockayne's syndrome, Kearns-Sayre disease, Waardenburg syndrome, Usher's syndrome, or multiple lentigines syndrome, or a developmental neurological disorder, such as autism, or a congenital brain defect that causes mental retardation, such as Down's syndrome, Chiari Malformation, Colpocephaly, holoprosencephaly, lissencephaly, or megalencephaly, or a circulatory disorder, such as stroke.

By a “Vacuolar Sorting Protein 18,” “VPSP18 protein,” or a “VPSP18 polypeptide” is meant a polypeptide that has at least 65%, 70%, 80%, 90%, or 95% identity to the zebrafish VPSP18 sequence of SEQ ID NO:22 over a region spanning at least 500, 600, 700, 800, 900, or 974 contiguous amino acids. Desirably, a “VPSP18 protein” or a “VPSP18 polypeptide” is identical to the sequence of SEQ ID NO:22. Polypeptides encoded by splice variants of VPSP18 nucleic acid sequences, as well as polypeptides encoded by VPSP18 nucleic acid sequences containing a mutation, for example, a mutation resulting from the insertion of a virus, e.g., an insertion at nucleotide 2336 of the sequence of SEQ ID NO:21 as referred to herein, are also included in this definition. VPSP18 plays a role in pigmentation, iridophore development, or tectum development. Accordingly, a “VPSP18 protein,” or a “VPSP18 polypeptide” may be used as a marker for, or to prevent or treat, for example, disorders related to pigmentation, such as human pigmentary glaucoma, Wagner's disease, Cockayne's syndrome, Kearns-Sayre disease, or multiple lentigines syndrome, or a sensory disorder, such as Waardenburg syndrome, or Usher's syndrome, or a developmental neurological disorder, for example, autism, or a retinal disorder, such as retinitis, macular degeneration, Friedreich's ataxia, or Laurence-Moon syndrome.

By a “Pescadillo protein” or a “Pescadillo polypeptide” is meant a polypeptide encoded by a Pescadillo gene (e.g., GenBank Accession No. U77627). Polypeptides encoded by splice variants of Pescadillo nucleic acid sequences, as well as Pescadillo nucleic acid sequences containing a mutation, for example, a mutation resulting from the insertion of a virus, e.g., an insertion at approximately nucleotide 20 of the Pescadillo nucleic acid sequence of GenBank Accession No. U77627, are also included in this definition. In addition, a “Pescadillo protein,” or a “Pescadillo polypeptide,” as referred to herein, plays a role in embryonic organ formation and cell cycle checkpoints. Accordingly, a “Pescadillo protein,” or a “Pescadillo polypeptide” may be used as a marker for, or to prevent or treat, for example, a proliferative disorder, such as cancer or neuroblastoma.

By a “HNF1-β/vHNF1 protein,” or a “HNF1-β/vHNF1 polypeptide” is meant a polypeptide that has at least 80%, 85%, 90%, 95%, or 99% amino acid sequence identity to the zebrafish HNF1-β/vHNF1 polypeptide sequence of SEQ ID NO:24 iver at keast 300, 350, 400, 500, or 550 contiguous amino acids. Desirably, a “HNF1-β/vHNF1 protein” or a “HNF1-β/vHNF1 polypeptide” is identical to the sequence of SEQ ID NO:24. Polypeptides encoded by splice variants of HNF1-β/vHNF1 nucleic acid sequences, as well as by HNF1-β/vHNF1 nucleic acids containing a mutation, for example, a mutation resulting from the insertion of a virus, e.g., an insertion between nucleotides 1682 and 1683 of the sequence of SEQ ID NO:23, or at nucleotide 361 or 745, are also included in this definition. A “HNF1-β/vHNF1 protein” or a “HNF1-β/vHNF1 polypeptide,” as referred to herein, plays a role in kidney or pancreas development, as well as in patterning the hind-brain. Accordingly, a “HNF1-β/vHNF1 protein,” or a “HNF1-β/vHNF1 polypeptide” may be used a marker for, or to prevent or treat, for example, a pancreatic or kidney disorder, such as diabetes, polycystic kidney disease, or Bardet-Biedl syndrome, or a congenital brain defect that causes mental retardation, such as Down's syndrome, Chiari Malformation, Colpocephaly, Holoprosencephaly, Lissencephaly, or Megalencephaly.

By a “60S Ribosomal L35 protein,” or a “60S Ribosomal L35 polypeptide” is meant a polypeptide that has at least 92%, 95%, 97%, or 99% amino acid sequence identity to the zebrafish 60S Ribosomal L35 polypeptide over at least 50, 75, 100, or 123 contiguous amino acids encoded by the 60S ribosomal L35 polypeptide sequence of SEQ ID NO:26. Desirably, a “60S Ribosomal L35 protein” or a “60S Ribosomal L35 polypeptide” is identical to the sequence of SEQ ID NO:26. Polypeptides encoded by splice variants of 60S Ribosomal L35 nucleic acid sequences, as well as by 60S Ribosomal L35 nucleic acids containing a mutation, for example, a mutation resulting from the insertion of a virus, e.g., an insertion between nucleotides 30 and 31 of the sequence of SEQ ID NO:25, are also included in this definition. A “60S Ribosomal L35 protein” or a “60S Ribosomal L35 polypeptide,” as referred to herein, plays a role in brain, head and eye development. A 60S Ribosomal protein may also be involved in the development of somite boundaries. Accordingly, a “60S Ribosomal L35 protein” or a “60S Ribosomal L35 polypeptide” may be used as a marker for, or to prevent or treat, for example, brain and/or eye disorders, sach as Balci's syndrome, or a congenital brain defect that causes mental retardation, such as microencephaly, anencephaly, Down's syndrome, Chiari Malformation, Colpocephaly, holoprosencephaly, lissencephaly, or megalencephaly, or muscle disorders, for example, a congenital muscular dystrophy disorder.

By a “60S Ribosomal L44 protein,” or a “60S Ribosomal L44 polypeptide” is meant a polypeptide that has at least 98% or 99% amino acid sequence identity to the zebrafish 60S Ribosomal L44 polypeptide sequence of SEQ ID NO:28 over at least 50, 75, or 106 amino acids. Desirably, a “60S Ribosomal L44 protein” or a “60S Ribosomal L44 polypeptide” is identical to the sequence of SEQ ID NO:28. Polypeptides encoded by splice variants of 60S Ribosomal L44 nucleic acid sequences, as well as by 60S Ribosomal L44 nucleic acids containing a mutation, for example, a mutation resulting from the insertion of a virus, e.g., an insertion between nucleotides 195 and 196 of the nucleic acid sequence of SEQ ID NO:27, are also included in this definition. A “60S Ribosomal L44 protein” or a “60S Ribosomal L44 polypeptide,” as referred to herein, plays a role in brain development and, in zebrafish, in the formation of the yolk sac extension. Accordingly, the loss of a 60S Ribosomal L44 polypeptide in a cell may result in an organism with large brain ventricles and a defective yolk. Consequently, a “60S Ribosomal L44 protein” or a “60S Ribosomal L44 polypeptide” may be used as a marker for, or to prevent or treat, for example, a congenital brain defect that causes mental retardation, such as microencephaly, anencephaly, Down's syndrome, Chiari Malformation, Colpocephaly, holoprosencephaly, lissencephaly, or megalencephaly.

By a “CopZ1 protein,” or a “CopZ1 polypeptide” is meant a polypeptide that is, for example, identical to the sequence of SEQ ID NO:30. Polypeptides encoded by splice variants of copZ1 nucleic acid sequences, as well as by copZ1 nucleic acids containing a mutation, for example, a mutation resulting from the insertion of a virus, e.g., an insertion between nucleotides 90 and 91 of the sequence of SEQ ID NO:29, are also included in this definition. A “CopZ1 protein” or a “CopZ1 polypeptide,” as referred to herein, plays a role in maintaining the retina. The loss of a CopZ1 polypeptide in a cell may result in eye degeneration, particularly in the retinal pigmented epithelia. Accordingly, a “CopZ1 protein” or a “CopZ1 polypeptide” may be used as a marker for, or to prevent or treat, for example, retinitis pigmentosa, macular degeneration, Friedreich's ataxia, or Laurence-Moon syndrome.

By a “215 protein,” or a “215 polypeptide” is meant a polypeptide that has at least 78% amino acid sequence identity over at least 529 amino acids to the zebrafish 215 polypeptide sequence of SEQ ID NO:32. Desirably, a “215 protein” or a “215 polypeptide” is at least 78%, 80%, 85%, 90%, 95%, 97%, or 99% identical to the sequence of SEQ ID NO:31. In a more desirable embodiment, a “215 protein” or a “215 polypeptide” is identical to the sequence of SEQ ID NO:31 over at least 300, 400, 450, or 500 contiguous amino acids. Polypeptides encoded by splice variants of 215 nucleic acid sequences, as well as by 215 nucleic acids containing a mutation, for example, a mutation resulting from the insertion of a virus, e.g., an insertion between nucleotides 294 and 295 of the sequence of SEQ ID NO:31, are also included in this definition. A “215 protein” or a “215 polypeptide,” as referred to herein, plays a role in eye and jaw development. For example, in zebrafish, the loss of a 215 polypeptide in a cell may result in eyes that are at least 75% smaller than those of three day-old wild-type zebrafish, or a jaw that is at least 75% reduced when compared to that of a three day old wild-type zebrafish. Alternatively, the loss of a 215 protein may result in general underdevelopment or a bent ceratohyal. Accordingly, a “215 protein” or a “215 polypeptide” may be used as a marker for, or to prevent or treat, for example, a craniofacial defect, such as Apert, Crouzon, Pfeiffer and Saethre-Chotzen Syndromes, or an eye malformation syndrome, such as oculorenal syndrome or Reiger syndrome.

By a “307 protein,” or a “307 polypeptide” is meant a polypeptide that has at least 54% amino acid sequence identity over at least 199 amino acids to the zebrafish 307 polypeptide sequence of SEQ ID NO:34. Desirably, a “307 protein” or a “307 polypeptide” is at least 54%, 60%, 70%, 75%, 80%, 85%, 90%, or 95% identical to the sequence of SEQ ID NO:34 over at least 75, 100, 125, 150, 175, or 199 contiguous amino acids. In a more desirable embodiment, a “307 protein” or a “307 polypeptide” is identical to the sequence of SEQ ID NO:34. Polypeptides encoded by splice variants of 307 nucleic acid sequences, as well as by 307 nucleic acids containing a mutation, for example, a mutation resulting from the insertion of a virus, e.g., an insertion at nucleotide 176 of the sequence of SEQ ID NO:33, are also included in this definition. A “307 protein” or a “307 polypeptide,” as referred to herein, plays a role in cartilage or jaw development. In zebrafish, the loss of a 307 polypeptide in a cell may result in mutants with mandibular arches that fail to extend anteriorly, and branchial arches that are slightly misshapen when compared to wild-type zebrafish. Accordingly, a “307 protein” or a “307 polypeptide” may be used as a marker for, or to prevent or treat, for example, a craniofacial disorder, such as Apert, Crouzon, Pfeiffer or Saethre-Chotzen Syndromes, or a connective tissue disease, such as rheumatoid arthritis.

By a “572 protein,” or a “572 polypeptide” is meant a polypeptide that has at least 37% amino acid sequence identity over at least 196 amino acids to the zebrafish 572 polypeptide sequence of SEQ ID NO:36. Desirably, a “572 protein” or a “572 polypeptide” is at least 37%, 40%, 50%, 60%, 70%, 80%, 85%, 90%, 95%, 97%, or 99% identical to the sequence of SEQ ID NO:36 over at least 100, 125, 150, 175, or 196 contiguous amino acids. In a more desirable embodiment, a “572 protein,” or a “572 polypeptide” is identical to the sequence of SEQ ID NO:36. Polypeptides encoded by splice variants of 572 nucleic acid sequences, as well as by 572 nucleic acids containing a mutation, for example, a mutation resulting from the insertion of a virus, e.g., an insertion at nucleotide 277 of SEQ ID NO:35, are also included in this definition. A zebrafish “572 protein” or a “572 polypeptide,” as referred to herein, plays a role in jaw and branchial arch development. The loss of a zebrafish 572 polypeptide in a cell may result in mutants with shorter jaws and fragmented branchial arches when compared to wild-type zebrafish. Accordingly, a “572 protein” or a “572 polypeptide” may be used as a marker for, or to prevent or treat, for example, a connective tissue disorder, such as arthritis, rheumatoid arthritis, or juvenile rheumatoid arthritis, or a craniofacial defect, such as Apert, Crouzon, Pfeiffer or Saethre-Chotzen Syndromes.

By a “1116A protein,” or a “1116A polypeptide” is meant a polypeptide that has at least 42% amino acid sequence identity over at least 191 contiguous amino acids amino acids to the zebrafish 1116A polypeptide sequence of SEQ ID NO:38. Desirably, a “1116A protein” or a “1116A polypeptide” is at least 45%, 50%, 60%, 70%, 80%, 85%, 90%, or 95% identical to the sequence of SEQ ID NO:38 over at least 75, 100, 125, 150, 175, or 191 contiguous amino acids. In a more desirable embodiment a “1116A protein,” or a “1116A polypeptide” is identical to the sequence of SEQ ID NO:38. Polypeptides encoded by splice variants of 1116A nucleic acid sequences, as well as by 1116A nucleic acids containing a mutation, for example, a mutation resulting from the insertion of a virus, e.g., an insertion at nucleotide 135 of the 1116A nucleic acid sequence of SEQ ID NO:37 are also included in this definition. A “1116A protein” or a “1116A polypeptide,” as referred to herein, plays a role in jaw development. For example, the loss of a zebrafish 116A polypeptide in a cell may result in failure of the jaw to form in three-day old mutant zebrafish. Accordingly, a “1116A protein” or a “1116A polypeptide” may be used as a marker for, or to prevent or treat, a craniofacial disorder, for example, Apert, Crouzon, Pfeiffer and Saethre-Chotzen Syndromes.

By a “1548 protein,” or a “1548 polypeptide” is meant a polypeptide that has at least 76%, 80%, 85%, 90%, 95%, or 98% amino acid sequence identity over at least 500, 600, 700, 800, 900, 925, or 950 contiguous amino acids to the zebrafish 1548 polypeptide sequence of SEQ ID NO:40. Desirably, a “1548 protein” or a “1548 polypeptide” is identical to the sequence of SEQ ID NO:40. Polypeptides encoded by splice variants of 1548 nucleic acid sequences, as well as by 1548 nucleic acids containing a mutation, for example, a mutation resulting from the insertion of a virus, e.g., an insertion at nucleotide 85 of the 1548 nucleic acid sequence of SEQ ID NO:39 are also included in this definition. A zebrafish “1548 protein” or a “1548 polypeptide,” as referred to herein, plays a role in eye, head, heart, fin, or jaw development. The loss of a zebrafish 1548 polypeptide in a cell may result in an added structure attached to the parachordal of the neurocranium in three day-old mutant zebrafish. Accordingly, a “1548 protein” or a “1548 polypeptide” may be used as a marker for, or to prevent or treat, for example, a neurological disorder, such as Balci syndrome or Angelman syndrome, or a craniofacial defect such as Apert, Crouzon, Pfeiffer or Saethre-Chotzen Syndromes, or a congenital brain defect that causes mental retardation, such as microencephaly, anencephaly, Down's syndrome, Chiari Malformation, Colpocephaly, holoprosencephaly, lissencephaly, or megalencephaly, or a congenital heart defect, such as a ventricular or atrial septal defect, or a limb formation defect, such as achondroplasia, Ellis-van Creveld syndrome, Poland sequence, or Pfeiffer syndrome.

By a “Casein Kinase 1 α protein,” or a “Casein Kinase 1 α polypeptide” is meant a polypeptide encoded by a Casein Kinase 1 α gene that has at least 99% amino acid sequence identity over at least 275, 300, or 324 amino acids to the zebrafish Casein Kinase 1 α sequence of SEQ ID NO:42. Desirably, a “Casein Kinase 1 α protein” or a “Casein Kinase 1 α polypeptide” is at least 99% or even 100% identical to the sequence of SEQ ID NO:42. Polypeptides encoded by splice variants of Casein Kinase 1 α nucleic acid sequences, as well as by Casein Kinase 1 α nucleic acids containing a mutation, for example, a mutation resulting from the insertion of a virus, e.g., an insertion between nucleotides 730 and 731 of the Casein Kinase 1 α nucleic acid sequence of SEQ ID NO:41 are also included in this definition. A “Casein Kinase 1 α protein” or a “Casein Kinase 1 α polypeptide,” as referred to herein, plays a role in cartilage development. For example, in zebrafish, the loss of a Casein Kinase 1 α polypeptide in a cell may result in the retarded development of pectoral fins in three-day old mutant zebrafish. In addition, these some of these fins may be misshapen. Alcian blue staining shows that the cartilage of the fins, brancial arches, and jaw is wrinkled. Accordingly, a “Casein Kinase 1 α protein” or a “Casein Kinase 1 α polypeptide” may be used as a marker for, or to prevent or treat, a limb formation defect, for example, achondroplasia, Ellis-van Creveld syndrome, Poland sequence, or Pfeiffer syndrome, or a cartilage or connective tissue disease, such as arthritis, juvenile rheumatoid arthritis, or Marfan's syndrome, or a craniofacial defect such as Apert, Crouzon, Pfeiffer or Saethre-Chotzen Syndromes.

By a “Nodal-Related or Squint protein,” or a “Nodal-Related or Squint polypeptide” is meant a polypeptide that has at least 42% amino acid sequence identity over at least 381 amino acids to the zebrafish “Nodal-Related or Squint protein sequence of SEQ ID NO:44. Desirably, a “Nodal-Related or Squint protein” or a “Nodal-Related or Squint polypeptide” is at least 43%, 50%, 60%, 70%, 80%, 90%, or even 100% identical to the sequence of SEQ ID NO:44. Polypeptides encoded by splice variants of Nodal-Related or Squint nucleic acid sequences, as well as by Nodal-Related or Squint nucleic acids containing a mutation, for example, a mutation resulting from the insertion of a virus, e.g., an insertion at nucleotide 654 of the Nodal-Related or Squint nucleic acid sequence of SEQ ID NO:43 are also included in this definition.

By a “Smoothened protein,” or a “Smoothened polypeptide” is meant a polypeptide that is, for example, identical to the zebrafish Smoothened amino acid sequence of SEQ ID NO:46. Polypeptides encoded by splice variants of Smoothened nucleic acid sequences, as well as by Smoothened nucleic acids containing a mutation, for example, a mutation resulting from the insertion of a virus, e.g., an insertion at nucleotide 271 the Smoothened nucleic acid sequence of SEQ ID NO:45 are also included in this definition. A “Smoothened protein” or a “Smoothened polypeptide,” as referred to herein, plays a role in in the development of motoneurons, forebrain and midbrain commissures, body shape/axial structures, cartilage, or muscles, also, optic nerves fail to reach or cross the midline. Accordingly, a “Smoothened” or a “Smoothened polypeptide” may be used as a marker for, or to prevent or treat, for example, a congenital disorder associated with mental retardation, such as microencephaly, anencephaly, Down's syndrome, Chiari Malformation, Colpocephaly, holoprosencephaly, lissencephaly, or megalencephaly, or a disorder affecting the cartilage or connective tissue, such as arthritis, juvenile rheumatoid arthritis, or Marfan's syndrome, or a disorder affecting muscle development, or a limb formation defect, such as achondroplasia, Ellis-van Creveld syndrome, Poland sequence, or Pfeiffer syndrome

By a “429 protein,” or a “429 polypeptide” is meant a polypeptide that has at least 53% amino acid sequence identity to the zebrafish 429 amino acid sequence of SEQ ID NO:48. Desirably, a “429 protein” or a “429 polypeptide” is at least 60%, 70%, 80%, 85%, 90%, 95%, 97%, 99%, or even 100% identical to the sequence of SEQ ID NO:48 over at least 500, 600, 700, or 750 contiguous amino acids. Polypeptides encoded by splice variants of 429 nucleic acid sequences, as well as by 429 nucleic acids containing a mutation, for example, a mutation resulting from the insertion of a virus, e.g., an insertion between nucleotides 182 and 183 of the 429 nucleic acid sequence of SEQ ID NO:47 are also included in this definition. A “429 protein” or a “429 polypeptide,” as referred to herein, plays a role in liver, gall bladder, pancreas, and gut development. The loss of a 429 polypeptide in a cell may result in the retarded development of these organs in three-day old mutant zebrafish. Accordingly, a “429 protein” or a “429 polypeptide” may be used as a marker for, or to prevent or treat, for example, a pancreatic disorder, such as diabetes.

By a “428 protein,” or a “428 polypeptide” is meant a polypeptide that has at least 62% amino acid sequence identity to the zebrafish 428 amino acid sequence of SEQ ID NO:50 over at least 170 amino acids. Desirably, a “428 protein” or a “428 polypeptide” is at least 62%, 70%, 80%, 85%, 90%, 95%, 97%, 99%, or even 100% identical to the sequence of SEQ ID NO:50 over at least 75, 100, 125, 150, 179 contiguous amino acids. Polypeptides encoded by splice variants of 428 nucleic acid sequences, as well as by 428 nucleic acids containing a mutation, for example, a mutation resulting from the insertion of a virus, e.g., an insertion at nucleotide 187 of the 428 nucleic acid sequence of SEQ ID NO:49 are also included in this definition. A “428 protein” or a “428 polypeptide,” as referred to herein, plays a role in muscle and brain development. The loss of a 428 polypeptide in a cell may result in defective muscles or brain necrosis. Accordingly, a “428 protein” or a “428 polypeptide” may be used as a marker for, or to prevent or treat, for example, a necrotizing brain disorder, such as Leigh's disease, or subacute necrotizing encephalomyelopathy, or a congenital brain defect that causes mental retardation, such as microencephaly, anencephaly, Down's syndrome, Chiari malformation, colpocephaly, holoprosencephaly, lissencephaly, or megalencephaly, or a muscle defect, such as myotonia congenital, inclusion body myositis, or a congenital muscular dystrophy.

By a “Spinster protein,” or a “Spinster polypeptide” is meant a polypeptide that has at least 64% amino acid sequence identity to the zebrafish Spinster amino acid sequence of SEQ ID NO:52 over at least 528 contiguous amino acids. Desirably, a “Spinster protein” or a “Spinster polypeptide” is at least 70%, 80%, 85%, 90%, 95%, 97%, 99%, or even 100% identical to the sequence of SEQ ID NO:52 over at least 300, 400, 450, or 507 contiguous amino acids. Polypeptides encoded by splice variants of Spinster nucleic acid sequences are also included in this definition. In addition, mutations Spinster nucleic acids that result in altered expression of a Spinster polypeptide, for example, a insertion of a virus several kb, for example, 2, 3, 4, 5, 8, 10, or even 15 kb upstream of the Spinster coding region (e.g., SEQ ID NO:51), are also included in this definition. A “Spinster protein” or a “Spinster polypeptide,” as referred to herein, plays a role in the maintainance of the yolk. In zebrafish, the loss of a Spinster polypeptide in a cell may result in the degeneration of the yolk by day two of development, resulting in the gradual death of the mutant zebrafish embryo. Accordingly, a “Spinster protein” or a “Spinster polypeptide” may be used as a marker for, or to prevent or treat, for example, a fertility defect.

By a “Glypican-6 or Knypek protein,” or a “Glypican-6 or Knypek polypeptide” is meant a polypeptide encoded by a Glypican-6 or Knypek gene, that has at least 58% amino acid sequence identity to the zebrafish Glypican-6 or Knypek amino acid sequence of SEQ ID NO:54 over a region spanning at least 550 amino acids. Desirably, a “Glypican-6 or Knypek protein” or a “Glypican-6 or Knypek polypeptide” is at least 60%, 70%, 80%, 85%, 90%, 95%, 97%, 99%, or even 100% identical to the sequence of SEQ ID NO:54 over at least 300, 350, 400, 450, 500, or 557 contiguous amino acids. Polypeptides encoded by splice variants of Glypican-6 or Knypek nucleic acid sequences, as well as by Glypican-6 or Knypek nucleic acids containing a mutation, for example, a mutation resulting from the insertion of a virus at nucleotide 1054 or nucleotide 133 of a Glypican-6 or Knypek nucleic acid sequence (e.g., SEQ ID NO:53) are also included in this definition. A “Glypican-6 or Knypek protein” or a “Glypican-6 or Knypek polypeptide,” as referred to herein, plays a role in the tail and somite development. For example, in zebrafish, the loss of a Glypican-6 or Knypek polypeptide in a cell may result in a shortened tail or u-shaped somites. Accordingly, a “Glypican-6 or Knypek protein” or a “Glypican-6 or Knypek polypeptide” may be used as a marker for, or to prevent or treat, for example, a muscle defect such as myotonia congenital, inclusion body myositis, or a congenital muscular dystrophy.

By a “Ribonucleotide Reductase R1 Class 1 protein,” or a “Ribonucleotide Reductase R1 Class 1 polypeptide” is meant a polypeptide encoded by a Ribonucleotide Reductase R1 Class 1 nucleic acid sequence that is identical to the zebrafish Ribonucleotide Reductase R1 Class 1 amino acid sequence of SEQ ID NO:56. Polypeptides encoded by splice variants of Ribonucleotide Reductase R1 Class 1 nucleic acid sequences, as well as by nucleic acids containing a mutation, for example, a mutation resulting from the insertion of a virus between nucleotides 147 and 148 of the Ribonucleotide Reductase R1 Class I nucleic acid sequence (e.g., SEQ ID NO:55) are also included in this definition. A “Ribonucleotide Reductase R1 Class 1 protein” or a “Ribonucleotide Reductase R1 Class 1 polypeptide,” as referred to herein, plays multiple roles in development. The loss of a Ribonucleotide Reductase R1 Class 1 polypeptide in a cell may result in a bent convex body shape. In addition, zebrafish mutant for a Ribonucleotide Reductase R1 Class 1 nucleic acid sequence display transient brain and eye necrosis between 24 and 48 hours of development. Accordingly, a “Ribonucleotide Reductase R1 Class 1 protein” or a “Ribonucleotide Reductase R1 Class 1 polypeptide” may be used as a marker for, or to prevent or treat, for example, a necrotizing brain disorder, such as Leigh's disease, or subacute necrotizing encephalomyelopathy, or a degenerative eye disorder, such as retinitis pigmentosa or other retinal disorders, such as macular degeneration, Friedreich's ataxia, or Laurence-Moon syndrome.

By a “Kinesin-Related Motor Protein EG5,” or a “Kinesin-Related Motor EG5 polypeptide” is meant a polypeptide that has at least 55% amino acid sequence identity to the zebrafish Kinesin-Related Motor Protein EG5 amino acid sequence of SEQ ID NO:58 over a region spanning at least 948 contiguous amino acids. Desirably, a “Kinesin-Related Motor Protein EG5” or a “Kinesin-Related Motor EG5 polypeptide” is at least 60%, 70%, 80%, 85%, 90%, 95%, 97%, 99%, or even 100% identical to the sequence of SEQ ID NO:58 over at least 700, 750, 800, 850, 900, or 948 contiguous amino acids. Polypeptides encoded by splice variants of Kinesin-Related Motor EG5 nucleic acid sequences, as well as by Kinesin-Related Motor EG5 nucleic acids containing a mutation, for example, a mutation resulting from the insertion of a virus between nucleotides 50 and 51 of a Kinesin-Related Motor EG5 nucleic acid sequence (e.g., SEQ ID NO:57) are also included in this definition. A “Kinesin-Related Motor protein EG5” or a “Kinesin-related motor EG5 polypeptide,” as referred to herein, plays a role in cell death during development. In zebrafish, the loss of a Kinesin-Related Motor EG5 polypeptide in a cell results in a bent body shape. In addition, these zebrafish mutants display elevated levels of apoptotic cells on their surface by 48 hours of development. Accordingly, a “Kinesin-Related Motor Protein EG5” or a “Kinesin-Related Motor EG5 polypeptide” may be used as a marker for, or to prevent or treat, a neurodegenerative disease characterized by excess cell death, for example, Alzheimer's disease, Parkinson's disease, or spinocerebellar ataxia, or to treat a proliferative disorder, such as cancer.

By a “Wnt5 or Pipetail protein,” or a “Wnt5 or Pipetail polypeptide” is meant a polypeptide that is identical to a zebrafish Pipetail amino acid sequence, for example, that of SEQ ID NO:62. Polypeptides encoded by splice variants of Wnt5 or Pipetail nucleic acid sequences (e.g., SEQ ID NO:61), as well as by Wnt5 or Pipetail nucleic acids containing a mutation, for example, a mutation resulting from the insertion of a virus at nucleotide 397, or between nucleotides 530 and 531 of the Wnt5 or Pipetail nucleic acid sequence of SEQ ID NO:61 nucleic acid sequence are also included in this definition. A “Wnt5 or Pipetail protein” or a “Wnt5 or Pipetail polypeptide,” as referred to herein, plays a role in cell death during development. For example, the loss of a Wnt5 or Pipetail polypeptide in a cell may result in a variably truncated tail in zebrafish. Accordingly, a “Wnt5 or Pipetail protein” or a “Wnt5 or Pipetail polypeptide” may be used as a marker for, or to prevent or treat, for example, a neurodegenerative disease characterized by excess cell death, for example, Alzheimer's disease, Parkinson's disease, or spinocerebellar ataxia.

By an “Aryl Hydrocarbon Receptor Nuclear Translocator 2A protein,” or an “Aryl Hydrocarbon Receptor Nuclear Translocator 2A polypeptide” is meant a polypeptide that, desirably, is identical to the sequence of SEQ ID NO:64. However, polypeptides encoded by splice variants of Aryl Hydrocarbon Receptor Nuclear Translocator 2A nucleic acid sequences, as well as by Aryl Hydrocarbon Receptor Nuclear Translocator 2A nucleic acids containing a mutation, for example, a mutation resulting from the insertion of a virus at nucleotide 229 or 240 of the Aryl Hydrocarbon Receptor Nuclear Translocator 2A nucleic acid sequence of SEQ ID NO:63 are also included in this definition. An “Aryl Hydrocarbon Receptor Nuclear Translocator 2A protein” or an “Aryl Hydrocarbon Receptor Nuclear Translocator 2A polypeptide,” as referred to herein, plays a role in cell death during development. For example, the loss of an Aryl Hydrocarbon Receptor Nuclear Translocator 2A polypeptide in a cell may result in little motility and a weak tap response in zebrafish. Accordingly, an “Aryl Hydrocarbon Receptor Nuclear Translocator 2A protein” or an “Aryl Hydrocarbon Receptor Nuclear Translocator 2A polypeptide” may be used as a marker for, or to prevent or treat, for example, a neurodegenerative disease, for example, Alzheimer's disease, Parkinson's disease, or spinocerebellar ataxia, or a movement disorder, such as Angelman's syndrome, tardive dyskinesia, or spasticity.

By a “Vesicular Integral-Membrane Protein VIP 36 Protein,” or a “Vesicular Integral-Membrane Protein VIP 36 polypeptide” is meant a polypeptide that has at least 61% amino acid sequence identity to the zebrafish Vesicular Integral-Membrane Protein VIP 36 amino acid sequence of SEQ ID NO:66 over a region spanning at least 340 contiguous amino acids. Desirably, a “Vesicular Integral-Membrane Protein VIP 36 protein” or a “Vesicular Integral-Membrane Protein VIP 36 polypeptide” is at least 61%, 70%, 80%, 90%, or 95% identical to the sequence of SEQ ID NO:66 over at least 250, 275, 300, o4 340 contiguous amino acids. In a more desirable embodiment, a “Vesicular Integral-Membrane Protein VIP 36 Protein,” or a “Vesicular Integral-Membrane Protein VIP 36 polypeptide” is identical to the sequence of SEQ ID NO:66. Polypeptides encoded by splice variants of Vesicular Integral-Membrane Protein VIP 36 nucleic acid sequences, as well as by Vesicular Integral-Membrane Protein VIP 36 nucleic acids containing a mutation, for example, a mutation resulting from the insertion of a virus between nucleotides 219 and 220 of the Vesicular Integral-Membrane Protein VIP 36 nucleic acid sequence of SEQ ID NO:65 are also included in this definition. A “Vesicular Integral-Membrane Protein VIP 36 protein” or a “Vesicular Integral-Membrane Protein VIP 36 polypeptide,” as referred to herein, plays a role touch sensitivity. For example, the loss of a Vesicular Integral-Membrane Protein VIP 36 polypeptide in a cell may result in touch insensitivity in zebrafish mutants at day five of development. Accordingly, a “Vesicular Integral-Membrane Protein VIP 36 protein” or a “Vesicular Integral-Membrane Protein VIP 36 polypeptide” may be used as a marker for, or to prevent or treat, for example, a neurodegenerative disease, such as Alzheimer's disease, Parkinson's disease, or spinocerebellar ataxia.

By a “299 Protein,” or a “299 polypeptide” is meant a polypeptide that has at least 44% amino acid sequence identity to the zebrafish 299 amino acid sequence of SEQ ID NO:68 over a region spanning 563 amino acids. Desirably, a “299” protein or a “299 polypeptide” is at least 44%, 50%, 60%, 70%, 80%, 90%, 95%, 99%, or even 100% identical to the sequence of SEQ ID NO:68. Polypeptides encoded by splice variants of 299 nucleic acid sequences, as well as by 299 nucleic acids containing a mutation, for example, a mutation resulting from the insertion of a virus between nucleotides 47 and 48 of the 299 nucleic acid sequence of SEQ ID NO:67 are also included in this definition. A “299 protein” or a “299 polypeptide,” as referred to herein, plays a role in cell death. For example, in zebrafish, the loss of a 299 polypeptide in a cell results in mutants that fail to develop a jaw, branchial arches, and normal size fins by day four of development and that have apoptosis in the eye and brain. Accordingly, a “299 protein” or a “299 polypeptide” may be used as a marker for, or to prevent or treat, for example, a neurodegenerative disease characterized by excess cell death, such as Alzheimer's disease, Parkinson's disease, or spinocerebellar ataxia, or a craniofacial defect, such as Apert, Crouzon, Pfeiffer and Saethre-Chotzen Syndromes, or a connective tissue disease, such as arthritis or rheumatoid arthritis, or may be used to treat a proliferative disorder, such as cancer.

By a “994 Protein,” or a “994 polypeptide” is meant a polypeptide that has at least 35% amino acid sequence identity to the zebrafish 994 amino acid sequence of SEQ ID NO:70 over a region spanning at least 490 contiguous amino acids. Desirably, a “994 protein” or a “994 polypeptide” is at least 35%, 40%, 50%, 60%, 70%, 80%, 90%, or 95% identical to the sequence of SEQ ID NO:70 over at least 300, 350, 400, 450, or 490 contiguous amino acids. In a more desirable embodiment, a “994 Protein,” or a “994 polypeptide” is identical to the sequence of SEQ ID NO:70. Polypeptides encoded by splice variants of 994 nucleic acid sequences, as well as by 994 nucleic acids containing a mutation, for example, a mutation resulting from the insertion of a virus between nucleotides 66 and 67 of a 994 nucleic acid sequence (e.g., SEQ ID NO:69) are also included in this definition. The coding region of the 994 gene may begin at nucleotide 5 or 80 of SEQ ID NO 69. In addition, a “994 protein” or a “994 polypeptide,” as referred to herein, plays a role in head and eye development. For example, in zebrafish, the loss of a 994 polypeptide in a cell results in mutants that have defects in jaw, branchial arch, fin development, an under developed stomach, and small heads and eyes. Accordingly, a “994 protein” or a “994 polypeptide” may be used as a marker for, or to prevent or treat, for example, a craniofacial disorder, such as Apert, Crouzon, Pfeiffer or Saethre-Chotzen Syndromes, or an eye malformation syndrome, such as oculorenal syndrome, or Reiger syndrome, or a limb formation defect, such as achondroplasia, Ellis-van Creveld syndrome, Poland sequence, or Pfeiffer syndrome, or a congenital brain defect that causes mental retardation, such as microencephaly, anencephaly, Down's syndrome, Chiari Malformation, Colpocephaly, holoprosencephaly, lissencephaly, or megalencephaly.

By a “1373 protein,” or a “1373 polypeptide” is meant a polypeptide encoded by a 1373 nucleic acid sequence that has at least 91% amino acid sequence identity to the zebrafish 1373 amino acid sequence of SEQ ID NO:72 over a region spanning at least 110 contiguous amino acids. Desirably, a “1373 protein” or a “1373 polypeptide” is at least 91% or 95% identical to the sequence of SEQ ID NO:72 over at least 75, 100, or 110 contiguous amino acids. In a more desirable embodiment, a “1373 protein,” or a “1373 polypeptide” is identical to the sequence of SEQ ID NO:72. Polypeptides encoded by splice variants of 1373 nucleic acid sequences, as well as by 1373 nucleic acids containing a mutation, for example, a mutation resulting from the insertion of a virus between nucleotides 118 and 119 of the 1373 nucleic acid sequence of SEQ ID NO:71 are also included in this definition. A “1373 protein” or a “1373 polypeptide,” as referred to herein, plays a role in development. For example, in zebrafish, the loss of a 1373 polypeptide in a cell results in mutants that display brain and eye necrosis, constriction of the anterior end of the yolk sac extension, and body curvature by day two of development. Accordingly, a “1373 protein” or a “1373 polypeptide” may be used as a marker for, or to prevent or treat, for example, a necrotizing brain disorder, Leigh's disease, or subacute necrotizing encephalomyelopathy, or a degenerative eye disorder, such as such as retinitis pigmentosa, macular degeneration, Friedreich's ataxia, or Laurence-Moon syndrome.

By a “Denticleless Protein,” or a “Denticleless polypeptide” is meant a polypeptide that has at least 46% amino acid sequence identity to the zebrafish Denticleless amino acid sequence of SEQ ID NO:74 over a region spanning at least 729 contiguous amino acids. Desirably, a “Denticleless protein” or a “denticleless polypeptide” is at least 46%, 50%, 60%, 70%, 80%, 90%, or 95% identical to the sequence of SEQ ID NO:74 over at least 500, 559, 600, 650, 700 or 729 contiguous amino acids. In a more desirable embodiment, a “Denticleless Protein,” or a “Denticleless polypeptide” is identical to the sequence of SEQ ID NO:74. Polypeptides encoded by splice variants of Denticleless nucleic acid sequences, as well as by Denticleless nucleic acids containing a mutation, for example, a mutation resulting from the insertion of a virus between nucleotides 307 and 308 of the Denticleless nucleic acid sequence of SEQ ID NO:73 are also included in this definition. A “Denticleless protein” or a “Denticleless polypeptide,” as referred to herein, plays a role in development. For example, in zebrafish, the loss of a Denticleless polypeptide in a cell results in mutants that display brain necrosis extending down the neural tube and a wrinkled yolk sac on day one of development. In addition, body curvature, wrinkled somites, irregular eye shape and the absence of the yolk sac extension are observed by day two of development. Accordingly, a “Denticleless protein” or a “Denticleless polypeptide” may be used as a marker for, or to prevent or treat, for example, a craniofacial disorder, such as Angelman's disease, or an eye malformation syndrome, for example, oculorenal syndrome, or Reiger syndrome, or a necrotizing brain disorder, such as Leigh's disease, or subacute necrotizing encephalomyelopathy.

By a “Ribonucleotide Reductase Protein R2 Protein,” or a “Ribonucleotide Reductase Protein R2 polypeptide” is meant, for example, a polypeptide that is identical to the sequence of SEQ ID NO:76. However, polypeptides encoded by splice variants of Ribonucleotide Reductase Protein R2 nucleic acid sequences, as well as by Ribonucleotide Reductase Protein R2 nucleic acids containing a mutation, for example, a mutation resulting from the insertion of a virus at nucleotide 137 (which corresponds to position 360 of an alternatively splice form of this gene (GenBank Accession No. AW280665)) of a Ribonucleotide Reductase Protein R2 nucleic acid sequence (e.g., SEQ ID NO:75), and virus insertions at nucleotide 337 or 342 of GenBank Accession No. AW280665, are also included in this definition. A “Ribonucleotide Reductase Protein R2 protein” or a “Ribonucleotide Reductase Protein R2 polypeptide,” as referred to herein, plays a role in development. For example, in zebrafish, the loss of a Ribonucleotide Reductase Protein R2 polypeptide in a cell results in mutants that display CNS necrosis and the entire body curls up by day two of development. Accordingly, a “Ribonucleotide Reductase Protein R2 protein” or a “Ribonucleotide Reductase Protein R2 polypeptide” may be used as a marker for, or to prevent or treat, for example, a necrotizing brain disorder, such as Leigh's disease, or subacute necrotizing encephalomyelopathy.

By a “TCP-1 Alpha protein,” or a “TCP-1 Alpha polypeptide” is meant a polypeptide that, desirably, is identical to the zebrafish TCP-1 Alpha amino acid sequence of SEQ ID NO:78. Polypeptides encoded by splice variants of TCP-1 Alpha nucleic acid sequences, as well as by TCP-1 Alpha nucleic acids containing a mutation, for example, a mutation resulting from the insertion of a virus between nucleotides 130 and 131, or 140 bp upstream of, a TCP-1 Alpha nucleic acid sequence (e.g., SEQ ID NO:77) are also included in this definition. A “TCP-1 Alpha protein” or a “TCP-1 Alpha polypeptide,” as referred to herein, plays a role in development. For example, in zebrafish, the loss of a TCP-1 Alpha polypeptide in a cell results in mutants that display small brains and eyes. Accordingly, a “TCP-1 Alpha protein” or a “TCP-1 Alpha polypeptide” may be used as a marker for, or to prevent or treat, for example, a congenital brain defect that causes mental retardation, such as anencephaly, Down's syndrome, Chiari Malformation, Colpocephaly, holoprosencephaly, lissencephaly, or megalencephaly, or a disorder associated with microcephaly, such as Angelman's syndrome or cri du chat, or an eye malformation syndrome, such as oculorenal syndrome or Reiger syndrome.

By a “Telomeric Repeat Factor 2 Protein,” or a “Telomeric Repeat Factor 2 polypeptide” is meant a polypeptide encoded by a Telomeric Repeat Factor 2 gene that has at least 32% amino acid sequence identity to the zebrafish Telomeric Repeat Factor 2 amino acids sequence of SEQ ID NO:80 over a region spanning 200 amino acids. Desirably, a “Telomeric Repeat Factor 2 protein” or a “Telomeric Repeat Factor 2 polypeptide” is at least 40%, 50%, 60%, 70%, 80%, 90%, or 95% identical to the sequence of SEQ ID NO:80 over at least 75, 100, 125, 150, 175, or 200 contiguous amino acids. In a more desirable embodiment, a “Telomeric Repeat Factor 2 Protein,” or a “Telomeric Repeat Factor 2 polypeptide” is identical to the sequence of SEQ ID NO:80. Polypeptides encoded by splice variants of Telomeric Repeat Factor 2 nucleic acid sequences, as well as by Telomeric Repeat Factor 2 nucleic acids containing a mutation, for example, a mutation resulting from the insertion of a virus between nucleotides 529 and 530 of the Telomeric Repeat Factor 2 nucleic acid sequence of SEQ ID NO:79 are also included in this definition. A “Telomeric Repeat Factor 2 protein” or a “Telomeric Repeat Factor 2 polypeptide,” as referred to herein, plays a role in brain and eye development. For example, in zebrafish, the loss of a Telomeric Repeat Factor 2 polypeptide in a cell results in mutants that display brain and eye necrosis by day two of development. Accordingly, a “Telomeric Repeat Factor 2 protein” or a “Telomeric Repeat Factor 2 polypeptide” may be used as a marker for, or to prevent or treat, for example, a necrotizing brain disorder, such as Leigh's disease, or subacute necrotizing encephalomyelopathy, or a degenerative eye disease, such as retinitis pigmentosa, macular degeneration, Friedreich's ataxia, or Laurence-Moon syndrome.

By a “SIL Protein,” or a “SIL polypeptide” is meant a polypeptide that has at least 36% amino acid sequence identity to the zebrafish SIL amino acid sequence of SEQ ID NO:82 over a region spanning 1363 amino acids. Desirably, a “SIL protein” or a “SIL polypeptide” is at least 36%, 50%, 60%, 70%, 80%, or 90% identical to the sequence of SEQ ID NO:82 over at least 700, 800, 900, 1000, 1100, 1200, 1300, or 1363 contiguous amino acids. In a more desirable embodiment, a “SIL Protein,” or a “SIL polypeptide” is identical to the sequence of SEQ ID NO:82. Polypeptides encoded by splice variants of SIL nucleic acid sequences, as well as by SIL nucleic acids containing a mutation, for example, a mutation resulting from the insertion of a virus between nucleotides 273 and 274 of the SIL nucleic acid sequence of SEQ ID NO:81 are also included in this definition. A “SIL protein” or a “SIL polypeptide,” as referred to herein, plays a role in development of the head and body. For example, in zebrafish, the loss of a SIL polypeptide in a cell results in mutants that have a head that is 33% smaller than wild-type by day two of development. In addition, these mutants exhibit brain necrosis, a bent body, and motility defects. Accordingly, a “SIL protein” or a “SIL polypeptide” may be used as a marker for, or to prevent or treat, for example, a necrotizing brain disorder, for example, Leigh's disease, or subacute necrotizing encephalomyelopathy, a congenital brain defect that causes mental retardation, such as anencephaly, Down's syndrome, Chiari Malformation, Colpocephaly, holoprosencephaly, lissencephaly, or megalencephaly, or a disorder associated with microcephaly, such as Angelman's syndrome or cri du chat, or an eye malformation syndrome, such as oculorenal syndrome or Reiger syndrome, or a degenerative eye disease, such as retinitis pigmentosa or other retinal disorders, such as macular degeneration, Friedrich ataxia, or Laurence-Moon syndrome.

By a “U1 Small Nuclear Ribonucleoprotein C Protein,” or a “U1 Small Nuclear Ribonucleoprotein C polypeptide” is meant a polypeptide that has at least 84% amino acid sequence identity to the zebrafish U1 Small Nuclear Ribonucleoprotein C amino acid sequence of SEQ ID NO:84 over a region spanning at least 159 contiguous amino acids. Desirably, a “U1 Small Nuclear Ribonucleoprotein C protein” or a “U1 Small Nuclear Ribonucleoprotein C polypeptide” is at least 90%, 95%, or 98% identical to the sequence of SEQ ID NO:84 over at least 75, 100, 125, or 159 contiguous amino acids. In a more desirable embodiment, a “U1 Small Nuclear Ribonucleoprotein C Protein,” or a “U1 Small Nuclear Ribonucleoprotein C polypeptide” is identical to the sequence of SEQ ID NO:84. Polypeptides encoded by splice variants of U1 Small Nuclear Ribonucleoprotein C nucleic acid sequences, as well as by U1 Small Nuclear Ribonucleoprotein C nucleic acids containing a mutation, for example, a mutation resulting from the insertion of a virus between nucleotides 52 and 53 of the U1 Small Nuclear Ribonucleoprotein C nucleic acid sequence of SEQ ID NO:83 are also included in this definition. A “U1 Small Nuclear Ribonucleoprotein C protein” or a “U1 Small Nuclear Ribonucleoprotein C polypeptide,” as referred to herein, plays a role in development of the brain, eyes and ears. For example, in zebrafish, the loss of a U1 Small Nuclear Ribonucleoprotein C polypeptide in a cell results in mutants that display motility defects, a body that curves upward, brain necrosis, smaller eyes and otoliths than wild-type zebrafish, pigment in the hind-brain, and retarded fin development. Accordingly, a “U1 Small Nuclear Ribonucleoprotein C protein” or a “U1 Small Nuclear Ribonucleoprotein C polypeptide” may be used as a marker for, or to prevent or treat, for example, a sensory disorder, such as Usher syndrome, Waardenburg syndrome, or other disorders characterized by congenital deafness or blindness, or an eye malformation syndrome, such as oculorenal syndrome or Reiger syndrome, or a necrotizing brain disorder, for example, Leigh's disease, or subacute necrotizing encephalomyelopathy, or a movement disorder, such as Angelman's syndrome, tardive dyskinesia, spasticity, ataxias, or spinocerebellar ataxia.

By a “Ski Interacting Protein,” or a “Ski Interacting polypeptide” is meant a polypeptide that has at least 84% amino acid sequence identity to the zebrafish Ski Interacting Protein amino acid sequence of SEQ ID NO:86 over a region spanning at least 536 contiguous amino acids. Desirably, a “Ski Interacting Protein protein” or a “Ski Interacting Protein polypeptide” is at least 90%, 95%, or 98% identical to the sequence of SEQ ID NO:86 over at least 300, 350, 400, 450, 500, or 536 contiguous amino acids. In a more desirable embodiment, a “Ski Interacting Protein,” or a “Ski Interacting polypeptide” is identical to the sequence of SEQ ID NO:86. Polypeptides encoded by splice variants of Ski Interacting Protein nucleic acid sequences, as well as by Ski Interacting Protein nucleic acids containing a mutation, for example, a mutation resulting from the insertion of a virus approximately 1.2 kb upstream from the beginning of the Ski Interacting Protein nucleic acid sequence of SEQ ID NO:85 are also included in this definition. A “Ski Interacting Protein” or a “Ski Interacting Protein polypeptide,” as referred to herein, plays a role in development of the brain and body. For example, in zebrafish, the loss of a Ski Interacting Protein polypeptide in a cell results in mutants that display extensive brain necrosis, a lack of brain divisions, a curved body, and abnormal motility by day two of development. Accordingly, a “Ski Interacting Protein protein” or a “Ski Interacting Protein polypeptide” may be used as a marker for, or to prevent or treat, for example, a necrotizing brain disorder, such as Leigh's disease, or subacute necrotizing encephalomyelopathy, or a congenital brain defect that causes mental retardation, such as microencephaly, anencephaly, Down's syndrome, Chiari Malformation, Colpocephaly, holoprosencephaly, lissencephaly, or megalencephaly, or a movement disorder such as Angelman's syndrome, tardive dyskinesia, spasticity, ataxias, or spinocerebellar ataxia.

By a “297 protein,” or a “297 polypeptide” is meant a polypeptide that has at least 77% amino acid sequence identity to the zebrafish 297 amino acid sequence of SEQ ID NO:88 over a region spanning at least 624 contiguous amino acids. Desirably, a “297 protein” or a “297 polypeptide” is at least 77%, 85%, 90%, or 95% identical to the sequence of SEQ ID NO:88 over at least 400, 500, 550, 600, or 624 contiguous amino acids. In a more desirable embodiment, “297 protein,” or a “297 polypeptide” is identical to the sequence of SEQ ID NO:88. Polypeptides encoded by splice variants of 297 nucleic acid sequences, as well as by 297 nucleic acids containing a mutation, for example, a mutation resulting from the insertion of a virus at nucleotide 74 of the 297 nucleic acid sequence of SEQ ID NO:87 are also included in this definition. A “297 protein” or a “297 polypeptide,” as referred to herein, plays a role in development of the brain, tail, cartilage, ethmoid plate, and the jaw. For example, in zebrafish, the loss of a 297 polypeptide in a cell results in mutants that display brain necrosis, and a kinked tail. Accordingly, a “297 protein” or a “297 polypeptide” may be used as a marker for, or to prevent or treat, for example, a necrotizing brain disorder, for example, Leigh's disease, or subacute necrotizing encephalomyelopathy, or a congenital brain defect that causes mental retardation, such as microencephaly, anencephaly, Down's syndrome, Chiari Malformation, Colpocephaly, holoprosencephaly, lissencephaly, or megalencephaly, or a craniofacial defect, such as Apert, Crouzon, Pfeiffer or Saethre-Chotzen Syndromes.

By a “TCP-1 Complex Gamma Chain protein,” or a “TCP-1 Complex Gamma Chain polypeptide” is meant a polypeptide that has at least 87% amino acid sequence identity to the zebrafish TCP-1 Complex Gamma Chain amino acid sequence of SEQ ID NO:90 over a region spanning at least 541 contiguous amino acids. Desirably, a “TCP-1 Complex Gamma Chain protein” or a “TCP-1 Complex Gamma Chain polypeptide” is at least 90%, 95%, or 98% identical to the sequence of SEQ ID NO:90 over at least 300, 350, 400, 450, 500, or 541 contiguous amino acids. In a more desirable embodiment, a “TCP-1 Complex Gamma Chain protein,” or a “TCP-1 Complex Gamma Chain polypeptide” is identical to the sequence of SEQ ID NO:90. Polypeptides encoded by splice variants of TCP-1 Complex Gamma Chain nucleic acid sequences, as well as by TCP-1 Complex Gamma Chain nucleic acids containing a mutation, for example, a mutation resulting from the insertion of a virus between nucleotides 75 and 76 of the TCP-1 Complex Gamma Chain nucleic acid sequence of SEQ ID NO:89 are also included in this definition. A “TCP-1 Complex Gamma Chain protein” or a “TCP-1 Complex Gamma Chain polypeptide,” as referred to herein, plays a role in development of the yolk sac. For example, in zebrafish, the loss of a TCP-1 Complex Gamma Chain polypeptide in a cell results in mutants that display a thinner yolk sac extension than wild-type.

By a “Small Nuclear Ribonucleoprotein D1 protein,” or a “Small Nuclear Ribonucleoprotein D1 polypeptide” is meant a polypeptide that has at least 97% amino acid sequence identity to the zebrafish Small Nuclear Ribonucleoprotein D1 amino acid sequence of SEQ ID NO:92 over at least 119 contiguous amino acids. Desirably, a “Small Nuclear Ribonucleoprotein D1 protein” or a “Small Nuclear Ribonucleoprotein D1 polypeptide” is at least 97%, 98%, 99%, or even 100% identical to the sequence of SEQ ID NO:92 over at least 75, 100, or 119 contiguous amino acids. Polypeptides encoded by splice variants of Small Nuclear Ribonucleoprotein D1 nucleic acid sequences, as well as by Small Nuclear Ribonucleoprotein D1 nucleic acids containing a mutation, for example, a mutation resulting from the insertion of a virus between nucleotides 76 and 77 of the Small Nuclear Ribonucleoprotein D1 nucleic acid sequence of SEQ ID NO:91 are also included in this definition. A “Small Nuclear Ribonucleoprotein D1 protein” or a “Small Nuclear Ribonucleoprotein D1 polypeptide,” as referred to herein, plays a role in development of the CNS. For example, in zebrafish, the loss of a Small Nuclear Ribonucleoprotein D1 polypeptide in a cell results in mutants that display an inflated hind-brain and increased necrosis in the CNS, particularly in the eye. Accordingly, a “Small Nuclear Ribonucleoprotein D1 protein” or a “Small Nuclear Ribonucleoprotein D1 polypeptide” may be used as a marker for, or to prevent or treat, for example, a necrotizing brain disorder, such as Leigh's disease, or subacute necrotizing encephalomyelopathy, or a degenerative eye disease, such as retinitis pigmentosa, macular degeneration, Friedreich's ataxia, or Laurence-Moon syndrome, or a congenital brain defect that causes mental retardation, such as microencephaly, anencephaly, Down's syndrome, Chiari Malformation, Colpocephaly, holoprosencephaly, lissencephaly, or megalencephaly.

By a “DNA Polymerase Epsilon Subunit B protein,” or a “DNA Polymerase Epsilon Subunit B polypeptide” is meant a polypeptide that has at least 74% amino acid sequence identity to the zebrafish DNA Polymerase Epsilon Subunit B amino acid sequence of SEQ ID NO:94 over at least 527 contiguous amino acids. Desirably, a “DNA Polymerase Epsilon Subunit B protein” or a “DNA Polymerase Epsilon Subunit B polypeptide” is at least 80%, 90%, 95% or 98% identical to the sequence of SEQ ID NO:94 over at least 300, 350, 400, 500, or 527 contiguous amino acids. In a more desirable embodiment, a “DNA Polymerase Epsilon Subunit B protein,” or a “DNA Polymerase Epsilon Subunit B polypeptide” is identical to the sequence of SEQ ID NO:94. Polypeptides encoded by splice variants of DNA Polymerase Epsilon Subunit B nucleic acid sequences, as well as by DNA Polymerase Epsilon Subunit B nucleic acids containing a mutation, for example, a mutation resulting from the insertion of a virus between nucleotides 1161 and 1162, or at nucleotide 929 of the DNA Polymerase Epsilon Subunit B nucleic acid sequence of SEQ ID NO:93 are also included in this definition. A “DNA Polymerase Epsilon Subunit B protein” or a “DNA Polymerase Epsilon Subunit B polypeptide,” as referred to herein, plays a role in development of the CNS. For example, in zebrafish, the loss of a DNA Polymerase Epsilon Subunit B polypeptide in a cell results in mutants that display increased necrosis in the brain and eye. Accordingly, a “DNA Polymerase Epsilon Subunit B protein” or a “DNA Polymerase Epsilon Subunit B polypeptide” may be used as a marker for, or to prevent or treat, for example, a necrotizing brain disorder, such as Leigh's disease, or subacute necrotizing encephalomyelopathy, or a degenerative eye disease, such as retinitis pigmentosa, macular degeneration, Friedreich's ataxia, or Laurence-Moon syndrome.

By an “821-02 protein,” or an “821-02 polypeptide” is meant a polypeptide that has at least 52% amino acid sequence identity to the zebrafish 821-02 amino acid sequence of SEQ ID NO:96 over at least 683 contiguous amino acids. Desirably, an “821-02 protein” or an “821-02 polypeptide” is at least 52%, 60%, 70%, 80%, 90%, 95%, or 98% identical to the sequence of SEQ ID NO:96 over at least 400, 450, 500, 550, 600, 650, or 683 contiguous amino acids. In a more desirable embodiment, an “821-02 protein,” or an “821-02 polypeptide” is identical to the sequence of SEQ ID NO:96. Polypeptides encoded by splice variants of 821-02 nucleic acid sequences, as well as by 821-02 nucleic acids containing a mutation, for example, a mutation resulting from the insertion of a virus between nucleotides 231 and 232, or between nucleotides 369 and 370, of the 821-02 nucleic acid sequence of SEQ ID NO:95 are also included in this definition. An “821-02 protein” or an “821-02 polypeptide,” as referred to herein, plays a role in cell death. For example, in zebrafish, the loss of an 821-02 polypeptide in a cell results in mutants that display extensive apoptosis in the CNS and the eye by 24 to 48 hours of development as visualized by acridine orange staining. Accordingly, an “821-02 protein” or an “821-02 polypeptide” may be used as a marker for, or to prevent or treat, a neurodegenerative disease characterized by excess cell death, for example, Alzheimer's disease, Parkinson's disease, or spinocerebellar ataxia, or a degenerative eye disease, such as retinitis pigmentosa, macular degeneration, Friedreich's ataxia, or Laurence-Moon syndrome.

By a “1045 protein,” or a “1045 polypeptide” is meant a polypeptide that has at least 76% amino acid sequence identity to the zebrafish 1045 amino acid sequence of SEQ ID NO:98 over a region that spans at least 265 contiguous amino acids. Desirably, a “1045 protein” or a “1045 polypeptide” is at least 80%, 90%, or 95% identical to the sequence of SEQ ID NO:98 over at least 150, 175, 200, 225, or 265 contiguous amino acids. In a more desirable embodiment, a “1045 protein,” or a “1045 polypeptide” is identical to the sequence of SEQ ID NO:98. Polypeptides encoded by splice variants of 1045 nucleic acid sequences, as well as by 1045 nucleic acids containing a mutation, for example, a mutation resulting from the insertion of a virus between nucleotides 216 and 344 of the 1045 nucleic acid sequence of SEQ ID NO:97 are also included in this definition. A “1045 protein” or a “1045 polypeptide,” as referred to herein, plays a role in brain development. For example, in zebrafish, the loss of a 1045 polypeptide in a cell results in mutants that display severe brain and head necrosis at 24 hours of development. Accordingly, a “1045 protein” or a “1045 polypeptide” may be used as a marker for, or to prevent or treat, for example, a necrotizing brain disorder, for example, Leigh's disease, or subacute necrotizing encephalomyelopathy.

By a “1055-1 protein,” or a “1055-1 polypeptide” is meant a polypeptide that has at least 70% amino acid sequence identity to the zebrafish 1055-1 amino acid sequence of SEQ ID NO:100 over a region that spans at least 285 contiguous amino acids. Desirably, a “1055-1 protein” or a “1055-1 polypeptide” is at least 70%, 75%, 80%, 90%, or 95% identical to the sequence of SEQ ID NO:100 over at least 150, 175, 200, 225, 250, or 285 contiguous amino acids. In a more desirable embodiment, sa “1055-1 protein,” or a “1055-1 polypeptide” is identical to the sequence of SEQ ID NO:100. Polypeptides encoded by splice variants of 1055-1 nucleic acid sequences, as well as by 1055-1 nucleic acids containing a mutation, for example, a mutation resulting from the insertion of a virus between nucleotides 167 and 168 of the 1055-1 nucleic acid sequence of SEQ ID NO:99 are also included in this definition. A “1055-1 protein” or a “1055-1 polypeptide,” as referred to herein, plays a role in yolk sac development. For example, in zebrafish, the loss of a 1055-1 polypeptide in a cell results in mutants that display a misshapen or missing yolk sac extension and a tail that bends down. A “1055-1 protein” or a “1055-1 polypeptide” may be used as a marker for, or to prevent or treat, a proliferative disorder.

By a “Spliceosome Associated Protein 49 protein,” or a “Spliceosome Associated Protein 49 polypeptide” is meant a polypeptide that has at least 80% amino acid sequence identity to the zebrafish Spliceosome Associated Protein 49 amino acid sequence of SEQ ID NO:102 over a region that spans at least 322 contiguous amino acids. Desirably, a “Spliceosome Associated Protein 49 protein” or a “Spliceosome Associated Protein 49 polypeptide” is at least 85%, 90%, 95% or 98% identical to the sequence of SEQ ID NO:102 over at least 200, 225, 250, or 300, or 322 contiguous amino acids. In a more desirable embodiment, a “Spliceosome Associated Protein 49 protein,” or a “Spliceosome Associated Protein 49 polypentide” is identical to the sequence of SEQ ID NO:102. Polypeptides encoded by splice variants of Spliceosome Associated Protein 49 nucleic acid sequences, as well as by Spliceosome Associated Protein 49 nucleic acids containing a mutation, for example, a mutation resulting from the insertion of a virus between nucleotides 53 and 54 of the Spliceosome Associated Protein 49 nucleic acid sequence of SEQ ID NO:101 are also included in this definition. A “Spliceosome Associated Protein 49 protein” or a “Spliceosome Associated Protein 49 polypeptide,” as referred to herein, plays a role in brain development. For example, in zebrafish, the loss of a Spliceosome Associated Protein 49 polypeptide in a cell results in mutants that display tectal necrosis and a bent body by day two of development. Accordingly, a “Spliceosome Associated Protein 49 protein” or a “Spliceosome Associated Protein 49 polypeptide” may be used as a marker for, or to prevent or treat, for example, a necrotizing brain disorder, such as Leigh's disease, or subacute necrotizing encephalomyelopathy.

By a “DNA Replication Licensing Factor MCM7 protein,” or a “DNA Replication Licensing Factor MCM7 polypeptide” is meant a polypeptide that has at least 75% amino acid sequence identity to the zebrafish DNA Replication Licensing Factor MCM7 amino acid sequence of SEQ ID NO:104 over at least 175, 100, 125, 150, 175, or 194 contiguous amino acids. Desirably, a “DNA Replication Licensing Factor MCM7 protein” or a “DNA Replication Licensing Factor MCM7 polypeptide” is at least 75%, 80%, 90%, or 95% identical to the sequence of SEQ ID NO:104. In a more desirable embodiment, a “DNA Replication Licensing Factor MCM7 protein” or a “DNA Replication Licensing Factor MCM7 polypeptide” is identical to the sequence of SEQ ID NO: 104. Polypeptides encoded by splice variants of DNA Replication Licensing Factor MCM7 nucleic acid sequences, as well as by DNA Replication Licensing Factor MCM7 nucleic acids containing a mutation, for example, a mutation resulting from the insertion of a virus between nucleotides 121 and 122, or at nucleotide 198, of the DNA Replication Licensing Factor MCM7 nucleic acid sequence of SEQ ID NO:103 are also included in this definition. A “DNA Replication Licensing Factor MCM7 protein” or a “DNA Replication Licensing Factor MCM7 polypeptide,” as referred to herein, plays a role in eye and CNS development. For example, in zebrafish, the loss of a DNA Replication Licensing Factor MCM7 polypeptide in a cell results in mutants that display severe eye and CNS necrosis during late day one, or early in day two of development. Accordingly, a “DNA Replication Licensing Factor MCM7 protein” or a “DNA Replication Licensing Factor MCM7 polypeptide” may be used as a marker for, or to prevent or treat, for example, a necrotizing brain disorder, such as Leigh's disease, or subacute necrotizing encephalomyelopathy disease, or a degenerative eye disease, such as retinitis pigmentosa, macular degeneration, Friedreich's ataxia, or Laurence-Moon syndrome.

By a “DEAD-Box RNA Helicase (DEAD5 or DEAD19) protein,” or a “DEAD-Box RNA Helicase (DEAD5 or DEAD19) polypeptide” is meant a polypeptide that has at least 84% amino acid sequence identity to the zebrafish DEAD-Box RNA Helicase (DEAD5 or DEAD19) amino acid sequence of SEQ ID NO:106 over a region that spans at least 487 contiguous amino acids. Desirably, a “DEAD-Box RNA Helicase (DEAD5 or DEAD19) protein” or a “DEAD-Box RNA Helicase (DEAD5 or DEAD19) polypeptide” is at least 84%, 90%, 95%, or 98% identical to the sequence of SEQ ID NO:106 over at least 300, 350, 400, 450, or 457 contiguous amino acids. In a more desirable embodiment, a “DEAD-Box RNA Helicase (DEAD5 or DEAD19) protein,” or a “DEAD-Box RNA Helicase (DEAD5 or DEAD19) polypeptide” is identical to the sequence of SEQ ID NO:106. Polypeptides encoded by splice variants of DEAD-Box RNA Helicase (DEAD5 or DEAD19) nucleic acid sequences, as well as by DEAD-Box RNA Helicase (DEAD5 or DEAD19) nucleic acids containing a mutation, for example, a mutation resulting from the insertion of a virus at nucleotide 132 of the DEAD-Box RNA Helicase (DEAD5 or DEAD19) nucleic acid sequence of SEQ ID NO:105 are also included in this definition. A “DEAD-Box RNA Helicase (DEAD5 or DEAD19) protein” or a “DEAD-Box RNA Helicase (DEAD5 or DEAD19) polypeptide,” as referred to herein, plays a role in brain development. For example, in zebrafish, the loss of a DEAD-Box RNA Helicase (DEAD5 or DEAD19) polypeptide in a cell results in mutants that display severe brain necrosis at 24 hours, and are dead by the second day of development. Accordingly, a “DEAD-Box RNA Helicase (DEAD5 or DEAD19) protein” or a “DEAD-Box RNA Helicase (DEAD5 or DEAD19) polypeptide” may be used as a marker for, or to prevent or treat, for example, a necrotizing brain disorder, such as Leigh's disease, or subacute necrotizing encephalomyelopathy disease.

By a “1581 protein,” or a “1581 polypeptide” is meant a polypeptide that has at least 48% amino acid sequence identity to the zebrafish 1581 amino acid sequence of SEQ ID NO:108 over a region that spans at least 273 contiguous amino acids. Desirably, a “1581 protein” or a “1581 polypeptide” is at least 55%, 60%, 70%, 75%, 80%, 85%, 90%, or 95% identical to the sequence of SEQ ID NO:108 over at least 150, 175, 200, 225, 250, or 273 contiguous amino acids. In a more desirable embodiment, a “1581 protein” or a “1581 polypeptide” is identical to the sequence of SEQ ID NO:108. Polypeptides encoded by splice variants of 1581 nucleic acid sequences, as well as by 1581 nucleic acids containing a mutation, for example, a mutation resulting from the insertion of a virus between nucleotides 346 and 347 of the 1581 nucleic acid sequence of SEQ ID NO:107 are also included in this definition. A “1581 protein” or a “1581 polypeptide,” as referred to herein, plays a role in brain and eye development. For example, in zebrafish, the loss of a 1581 polypeptide in a cell results in mutants that display brain and eye necrosis and brains and eyes that are 50% smaller than those of wild-type zebrafish by the third day of development. Accordingly, a “1581 protein” or a “1581 polypeptide” may be used as a marker for, or to prevent or treat, for example, a necrotizing brain disorder, such as Leigh's disease, or subacute necrotizing encephalomyelopathy, or an eye malformation syndrome, for example, oculorenal syndrome, or Reiger syndrome, or a congenital brain defect that causes mental retardation, such as microencephaly, anencephaly, Down's syndrome, Chiari Malformation, Colpocephaly, holoprosencephaly, lissencephaly, or megalencephaly.

By a “Cyclin A2 protein,” or a “Cyclin A2 polypeptide” is meant a polypeptide that is, for example, identical to the sequence of SEQ ID NO:110. Polypeptides encoded by splice variants of Cyclin A2 nucleic acid sequences, as well as by Cyclin A2 nucleic acids containing a mutation, for example, a mutation resulting from the insertion of a virus at nucleotide 374 or 401 of the Cyclin A2 nucleic acid sequence of SEQ ID NO:109 are also included in this definition. A “Cyclin A2 protein” or a “Cyclin A2 polypeptide,” as referred to herein, plays a role head development. For example, in zebrafish, the loss of a Cyclin A2 polypeptide in a cell results in mutants that display heads that are 66% smaller than those of wild-type zebrafish by the fifth day of development display eye and CNS necrosis, no jaw, and abnormal semicircular canals. Accordingly, a “Cyclin A2 protein” or a “Cyclin A2 polypeptide” may be used as a marker for, or to prevent or treat, for example, a proliferative disease, sensory disorders, for example, Usher syndrome, Waardenburg syndrome, or other disorders characterized by congenital deafness or blindness, or a neurodegenerative disease such as Alzheimer's disease, Parkinson's disease, or spinocerebellar ataxia, or a congenital brain defect that causes mental retardation, such as microencephaly, anencephaly, Down's syndrome, Chiari Malformation, Colpocephaly, holoprosencephaly, lissencephaly, or megalencephaly, or a necrotizing brain disorder, such as Leigh's disease, or subacute necrotizing encephalomyelopathy disease.

By an “Imitation Switch (ISWI)/SNF2 protein,” an “ISWI/SNF2 protein,” or an “Imitation Switch (ISWI)/SNF2 polypeptide,” or an “ISWI/SNF2 polypeptide” is meant a polypeptide that has at least 70% amino acid sequence identity to the zebrafish ISWI/SNF2 amino acid sequence of SEQ ID NO:112 over at least 75, 100, 125, or 145 contiguous amino acids. Desirably, an “ISWI/SNF2 protein” or an “ISWI/SNF2 polypeptide” is at least 70%, 80%, 85%, 90%, or 95% identical to the sequence of SEQ ID NO:112. In a more desirable embodiment, an “ISWI/SNF2 protein,” or an “ISWI/SNF2 polypeptide” is identical to the sequence of SEQ ID NO:112. Polypeptides encoded by splice variants of ISWI/SNF2nucleic acid sequences, as well as by ISWI/SNF2 nucleic acids containing a mutation, for example, a mutation resulting from the insertion of a virus at nucleotide 76 of the ISWI/SNF2 nucleic acid sequence of SEQ ID NO:111 are also included in this definition. An “ISWI/SNF2 protein” or an “ISWI/SNF2 polypeptide,” as referred to herein, plays a role in eye, brain and jaw development. For example, in zebrafish, the loss of an ISWI/SNF2 polypeptide in a cell results in mutants that display eye necrosis, and necrosis of the inner cell ganglion layer and the optic tectum. The eyes of these mutants are 25% smaller those of wild-type zebrafish by the fourth day of development, and their lower jaw has dropped. Accordingly, an “ISWI/SNF2 protein” or an “ISWI/SNF2 polypeptide” may be used as a marker for, or to prevent or treat, for example, a necrotizing brain disorder, such as Leigh's disease, or subacute necrotizing encephalomyelopathy, or an eye malformation syndrome, such as syndrome, or Reiger syndrome, or a degenerative eye disease, such as retinitis pigmentosa or other retinal disorders, for example, macular degeneration, Friedreich's ataxia, or Laurence-Moon syndrome.

By a “Chromosomal Assembly Protein C (XCAP-C) protein,” an “XCAP-C protein,” or a “Chromosomal Assembly Protein C (XCAP-C) polypeptide,” or an “XCAP-C polypeptide” is meant a polypeptide that has at least 70% amino acid sequence identity to the zebrafish XCAP-C amino acid sequence of SEQ ID NO:114 over at least 600, 700, 800, 900, or 979 contiguous amino acids. Desirably, a “XCAP-C protein” or a “XCAP-C polypeptide” is at least 70%, 75%, 80%, 85%, 90%, or 95% identical to the sequence of SEQ ID NO:114. In a more desirable embodiment, a “XCAP-C protein,” or a “XCAP-C polypeptide” is identical to the sequence of SEQ ID NO:114. Polypeptides encoded by splice variants of XCAP-C nucleic acid sequences, as well as by XCAP-C nucleic acids containing a mutation, for example, a mutation resulting from the insertion of a virus at nucleotide 181 and 182 of the XCAP-C nucleic acid sequence of SEQ ID NO: 113 are also included in this definition. A “XCAP-C protein” or a “XCAP-C polypeptide,” as referred to herein, plays a role in brain and eye development. For example, in zebrafish, the loss of a XCAP-C polypeptide in a cell results in mutants that display necrosis in the eye, optic tectum and hind-brain by day two of development. Accordingly, a “XCAP-C protein” or a “XCAP-C polypeptide” may be used as a marker for, or to prevent or treat, for example, a necrotizing brain disorder, such as Leigh's disease, or subacute necrotizing encephalomyelopathy, or a degenerative eye disease, such as retinitis piginentosa, macular degeneration, Friedreich's ataxia, or Laurence-Moon syndrome.

By a “DNA Replication Licensing Factor MCM2 protein,” or a “DNA Replication Licensing Factor MCM2 polypeptide” is meant a polypeptide that has at least 79% amino acid sequence identity to the zebrafish DNA Replication Licensing Factor MCM2 amino acid sequence of SEQ ID NO:116 over a region that spans at least 893 contiguous amino acids. Desirably, a “DNA Replication Licensing Factor MCM2 protein” or a “DNA Replication Licensing Factor MCM2 polypeptide” is at least 79%, 85%, 90%, or 95% identical to the sequence of SEQ ID NO:116 over at least 600, 700, 800, 850, or 893 contiguous amino acids. In a more desirable embodiment, a “DNA Replication Licensing Factor MCM2 protein,” or a “DNA Replication Licensing Factor MCM2 polypeptide” is identical to the sequence of SEQ ID NO:116. Polypeptides encoded by splice variants of DNA Replication Licensing Factor MCM2 nucleic acid sequences, as well as by DNA Replication Licensing Factor MCM2 nucleic acids containing a mutation, for example, a mutation resulting from the insertion of a virus at nucleotide 399 of the DNA Replication Licensing Factor MCM2 nucleic acid sequence of SEQ ID NO:115 are also included in this definition. A “DNA Replication Licensing Factor MCM2 protein” or a “DNA Replication Licensing Factor MCM2 polypeptide,” as referred to herein, plays a role in brain and eye development. For example, in zebrafish, the loss of a DNA Replication Licensing Factor MCM2 polypeptide in a cell results in mutants that display necrosis in the optic tectum. In addition the eyes of these mutants are smaller than those of wild-type zebrafish, and they have abnormal jaws and branchial arches by day five of development. Accordingly, a “DNA Replication Licensing Factor MCM2 protein” or a “DNA Replication Licensing Factor MCM2 polypeptide” may be used as a marker for, or to prevent or treat, a craniofacial defect, for example, Apert, Crouzon, Pfeiffer and Saethre-Chotzen Syndromes, or, for example, a necrotizing brain disorder, for example, Leigh's disease, or subacute necrotizing encephalomyelopathy, an eye malformation syndrome, such as oculorenal syndrome or Reiger syndrome, or a craniofacial defect, such as Apert, Crouzon, Pfeiffer or Saethre-Chotzen Syndromes.

By a “DNA Replication Licensing Factor MCM3 protein,” or a “DNA Replication Licensing Factor MCM3 polypeptide” is meant a polypeptide that has at least 86% amino acid sequence identity to the zebrafish DNA Replication Licensing Factor MCM3 amino acid sequence of SEQ ID NO:118 over a region that spans at least 178 contiguous amino acids. Desirably, a “DNA Replication Licensing Factor MCM3 protein” or a “DNA Replication Licensing Factor MCM3 polypeptide” is at least 86%, 90%, or 95% identical to the sequence of SEQ ID NO:118 over at least 100, 125, 150, or 178 contiguous amino acids. In a more desirable embodiment, a “DNA Replication Licensing Factor MCM3 protein,” or a “DNA Replication Licensing Factor MCM3 polypeptide” is identical to the sequence of SEQ ID NO:118. Polypeptides encoded by splice variants of DNA Replication Licensing Factor MCM3 nucleic acid sequences, as well as by DNA Replication Licensing Factor MCM3 nucleic acids containing a mutation, for example, a mutation resulting from the insertion of a virus at nucleotide 50, or between nucleotides 75 and 76 of the DNA Replication Licensing Factor MCM3 nucleic acid sequence of SEQ ID NO:117 are also included in this definition. A “DNA Replication Licensing Factor MCM3 protein” or a “DNA Replication Licensing Factor MCM3 polypeptide,” as referred to herein, plays a role in eye and brain development. For example, in zebrafish, the loss of a DNA Replication Licensing Factor MCM3 polypeptide in a cell results in mutants that display necrosis in the optic tectum. In addition the head and eyes of these mutants are at least 25% smaller than those of wild-type zebrafish by day three of development. Accordingly, a “DNA Replication Licensing Factor MCM3 protein” or a “DNA Replication Licensing Factor MCM3 polypeptide” may be used as a marker for, or to prevent or treat, a disorder associated with microcephaly, such as Angelman's syndrome or cri du chat, or an eye malformation syndrome, such as oculorenal syndrome or Reiger syndrome.

By a “Valyl-tRNA Synthase protein,” or a “Valyl-tRNA Synthase polypeptide” is meant a polypeptide that has at least 52% amino acid sequence identity to the zebrafish Valyl-tRNA Synthase amino acid sequence of SEQ ID NO:120 over at least 250, 300, 350, 400, or 440 contiguous amino acids. Desirably, a “Valyl-tRNA Synthase protein” or a “Valyl-tRNA Synthase polypeptide” is at least 52%, 60%, 70%, 80%, 90%, or 95% identical to the sequence of SEQ ID NO:120. In a more desirable embodiment, a “Valyl-tRNA Synthase protein,” or a “Valyl-tRNA Synthase polypeptide” is identical to the sequence of SEQ ID NO:120. Polypeptides encoded by splice variants of Valyl-tRNA Synthase nucleic acid sequences, as well as by Valyl-tRNA Synthase nucleic acids containing a mutation, for example, a mutation resulting from the insertion of a virus between nucleotides 30 and 31 of the Valyl-tRNA Synthase nucleic acid sequence of SEQ ID NO:119 are also included in this definition. A “Valyl-tRNA Synthase protein” or a “Valyl-tRNA Synthase polypeptide,” as referred to herein, plays a role in cell death or pigmentation. For example, in zebrafish, the loss of a Valyl-tRNA Synthase polypeptide in a cell results in mutants that display apoptosis in the brain. In addition, the head and eyes of these mutants are smaller than those of wild-type zebrafish by day three of development. The eyes of the mutant zebrafish are also lighter in color than those of wild-type zebrafish. Accordingly, a “Valyl-tRNA Synthase protein” or a “Valyl-tRNA Synthase polypeptide” may be used as a marker for, or to prevent or treat, for example, a disorder associated with microcephaly, such as Angelman's syndrome or cri du chat, or a neurodegenerative disease characterized by excess cell death, for example, Alzheimer's disease, Parkinson's disease, or spinocerebellar ataxia, or disorders related to pigmentation, such as human pigmentary glaucoma, Wagner's disease, Cockayne's syndrome, Kearns-Sayre disease, Waardenburg syndrome, Usher's syndrome, or Multiple lentigines syndrome.

By a “40S Ribosomal Protein S5 protein,” or a “40S Ribosomal Protein S5 polypeptide” is meant a polypeptide that has at least 96% amino acid sequence identity to the zebrafish 40S Ribosomal Protein S5 amino acid sequence of SEQ ID NO:122 over a region that spans at least 202 contiguous amino acids. Desirably, a “40S Ribosomal Protein S5 protein” or a “40S Ribosomal Protein S5 polypeptide” is at least 98% or 99% identical to the sequence of SEQ ID NO:122 over at least 100, 125, 150, 175, or 202 contiguous amino acids. In a more desirable embodiment, a “40S Ribosomal Protein S5 protein” or a “40S Ribosomal Protein S5 polypeptide” is idenical to SEQ ID NO:122. Polypeptides encoded by splice variants of 40S Ribosomal Protein S5 nucleic acid sequences, as well as by 40S Ribosomal Protein SS nucleic acids containing a mutation, for example, a mutation resulting from the insertion of a virus between nucleotides 31 and 32 of the 40S Ribosomal Protein SS nucleic acid sequence of SEQ ID NO:121 are also included in this definition. A “40S Ribosomal Protein S5 protein” or a “40S Ribosomal Protein S5 polypeptide,” as referred to herein, plays a role in brain development. For example, in zebrafish, the loss of a 40S Ribosomal Protein S5 polypeptide in a cell results in mutants that display a swollen “bubble-brain” phenotype at day two of development. In addition, the mutant zebrafish display persistent motility defects. Accordingly, a “40S Ribosomal Protein S5 protein” or a “40S Ribosomal Protein S5 polypeptide” may be used as a marker for, or to prevent or treat, for example, a congenital brain defect, such as hydranencephaly.

By a “TCP-1 Beta protein,” or a “TCP-1 Beta polypeptide” is meant a polypeptide that has at least 86% amino acid sequence identity to the zebrafish TCP-1 Beta amino acid sequence of SEQ ID NO:124 over a region that spans at least 507 contiguous amino acids. Desirably, a “TCP-1 Beta protein” or a “TCP-1 Beta polypeptide” is at least 90%, 95%, 98%, 99%, or even 100% identical to the sequence of SEQ ID NO:124 over at least 300, 350, 400, 450, or 507 contiguous amino acids. Polypeptides encoded by splice variants of TCP-1 Beta nucleic acid sequences, as well as by TCP-1 Beta nucleic acids containing a mutation, for example, a mutation resulting from the insertion of a virus between nucleotides 63 and 64 of the TCP-1 Beta nucleic acid sequence of SEQ ID NO: 123 are also included in this definition. A “TCP-1 Beta protein” or a “TCP-1 Beta polypeptide,” as referred to herein, plays a role in head development. For example, in zebrafish, the loss of a TCP-1 Beta polypeptide in a cell results in mutants that display jaw and cartilage defects, as well as a small head and eyes at day three of development as compared to wild-type zebrafish. Accordingly, a “TCP-1 Beta protein” or a “TCP-1 Beta polypeptide” may be used as a marker for, or to prevent or treat, for example, a disorder associated with microcephaly, such as Angelman's syndrome or cri du chat, or a disorder affecting the cartilage or connective tissue, such as arthritis, or a craniofacial defect, such as Apert, Crouzon, Pfeiffer and Saethre-Chotzen Syndromes, or an eye malformation syndrome, such as Reiger syndrome.

By a “TCP-1 Eta protein,” or a “TCP-1 Eta polypeptide” is meant a polypeptide that has at least 88% amino acid sequence identity to the zebrafish TCP-1 Eta amino acid sequence of SEQ ID NO:126 over a region that spans at least 541 contiguous amino acids. Desirably, a “TCP-1 Eta protein” or a “TCP-1 Eta polypeptide” is at least 88%, 90%, 95%, 98%, 99%, or even 100% identical to the sequence of SEQ ID NO:126 over at least 350, 400, 450, 500, or 541 contiguous amino acids. Polypeptides encoded by splice variants of TCP-1 Eta nucleic acid sequences, as well as by TCP-1 Eta nucleic acids containing a mutation, for example, a mutation resulting from the insertion of a virus between nucleotides 32 and 33 of the TCP-1 Eta nucleic acid sequence of SEQ ID NO:125 are also included in this definition. A “TCP-1 Eta protein” or a “TCP-1 Eta polypeptide,” as referred to herein, plays a role in head and eyedevelopment. For example, in zebrafish, the loss of a TCP-1 Eta polypeptide in a cell results in mutants that display, for example, smaller head and smaller eyes when compared to age-matched wild-type zebrafish. Accordingly, a “TCP-1 Eta protein” or a “TCP-1 Eta polypeptide” may be used as a marker for, or to prevent or treat, for example, a disorder associated with microcephaly, such as Angelman's syndrome or cri du chat, disease.

By a “Translation Elongation Factor eEF1 Alpha protein,” or a “Translation Elongation Factor eEF1 Alpha polypeptide” is meant a polypeptide that is, desirably, identical to the zebrafish Translation Elongation Factor eEF1 Alpha amino acid sequence of SEQ ID NO:128. Polypeptides encoded by splice variants of Translation Elongation Factor eEF1 Alpha nucleic acid sequences, as well as by Translation Elongation Factor eEF1 Alpha nucleic acids containing a mutation, for example, a mutation resulting from the insertion of a virus between nucleotides 60 and 61 of the Translation Elongation Factor eEF1 Alpha nucleic acid sequence of SEQ ID NO:127 are also included in this definition. A “Translation Elongation Factor eEF1 Alpha protein” or a “Translation Elongation Factor eEF1 Alpha polypeptide,” as referred to herein, plays a role in cell death. For example, in zebrafish, the loss of a Translation Elongation Factor eEF1 Alpha polypeptide in a cell results in mutants that display increased apoptosis in the head and eyes. These mutants display a head and eyes that are at least 33% smaller then those of age-matched wild-type zebrafish. Accordingly, a “Translation Elongation Factor eEF1 Alpha protein” or a “Translation Elongation Factor eEF1 Alpha polypeptide” may be used as a marker for, or to prevent or treat, for example, a neurodegenerative disease characterized by excess cell death, such as Alzheimer's disease, Parkinson's disease, or spinocerebellar ataxia, or a degenerative eye disease, such as retinitis pigmentosa, macular degeneration, Friedreich's ataxia, or Laurence-Moon syndrome, or a disorder associated with microcephaly, such as Angelman's syndrome or cri du chat.

By a “1257 protein,” or a “1257 polypeptide” is meant a polypeptide that has at least 49% amino acid sequence identity to the zebrafish 1257 amino acid sequence of SEQ ID NO:130 over a region spanning at least 372 contiguous amino acids. Desirably, a “1257 protein” or a “1257 polypeptide” is at least 49%, 60%, 70%, 80%, 90%, or 95% identical to the sequence of SEQ ID NO:130 over at least 250, 300, 350, or 372 contiguous amino acids. In a more desirable embodiment, a “1257 protein,” or a “1257 polypeptide” is identical to the sequence of SEQ ID NO:130. Polypeptides encoded by splice variants of 1257 nucleic acid sequences, as well as by 1257 nucleic acids containing a mutation, for example, a mutation resulting from the insertion of a virus at nucleotide 175 of the 1257 nucleic acid sequence of SEQ ID NO:129 are also included in this definition. A “1257 protein” or a “1257 polypeptide,” as referred to herein, plays a role in head and eye development. For example, in zebrafish, the loss of a 1257 polypeptide in a cell results in mutants having a head and eyes that are at least 25% smaller than those of age-matched four-day old wild-type zebrafish. In addition these zebrafish have an underdeveloped jaw. Accordingly, a “1257 protein” or a “1257 polypeptide” may be used as a marker for, or to prevent or treat, for example, a disorder associated with microcephaly, such as Angelman's syndrome or cri du chat, or an eye malformation syndrome, such as oculorenal syndrome, or Reiger syndrome, or a craniofacial defect, such as Apert, Crouzon, Pfeiffer or Saethre-Chotzen Syndromes.

By a “60S Ribosomal Protein L24 protein,” or a “60S Ribosomal Protein L24 polypeptide” is meant a polypeptide that has at least 89% amino acid sequence identity to the zebrafish 60S Ribosomal Protein L24 amino acid sequence of SEQ ID NO:132 over a region spanning at least 157 contiguous amino acids. Desirably, a “60S Ribosomal Protein L24 protein” or a “60S Ribosomal Protein L24 polypeptide” is at least 89%, 90%, or 95% identical to the sequence of SEQ ID NO:132 over at least 75, 100, 125, or 157 contiguous amino acids. In a more desirable embodiment, a “60S Ribosomal Protein L24 protein,” or a “60S Ribosomal Protein L24 polypeptide” is identical to the sequence of SEQ ID NO:132. Polypeptides encoded by splice variants of 60S Ribosomal Protein L24 nucleic acid sequences, as well as by 60S Ribosomal Protein L24 nucleic acids containing a mutation, for example, a mutation resulting from the insertion of a virus between nucleotides 144 and 145 of the 60S Ribosomal Protein L24 nucleic acid sequence of SEQ ID NO:131 are also included in this definition. A “60S Ribosomal Protein L24 protein” or a “60S Ribosomal Protein L24 polypeptide,” as referred to herein, plays a role in head development. For example, in zebrafish, the loss of a 60S Ribosomal Protein L24 polypeptide in a cell results in mutants that display having a head and eyes that are at least 33% smaller than those of age-matched wild-type zebrafish. Accordingly, a “60S Ribosomal Protein L24 protein” or a “60S Ribosomal Protein L24 polypeptide” may be used as a marker for, or to prevent or treat, for example, a disorder associated with microcephaly, such as Angelman's syndrome or cri du chat, or an eye malformation syndrome, such as oculorenal syndrome, or Reiger syndrome.

By a “Non-Muscle Adenylosuccinate Synthase protein,” or a “Non-Muscle Adenylosuccinate Synthase polypeptide” is meant a polypeptide that has at least 76% amino acid sequence identity to the zebrafish Non-Muscle Adenylosuccinate Synthase amino acid sequence of SEQ ID NO:134 over a region spanning at least 175 contiguous amino acids. Desirably, a “Non-Muscle Adenylosuccinate Synthase” or a “Non-Muscle Adenylosuccinate Synthase polypeptide” is at least 80%, 90%, or 95% identical to the sequence of SEQ ID NO:134 over at least 75, 100, 125, 150, or 175 contiguous amino acids. In a more desirable embodiment, a “Non-Muscle Adenylosuccinate Synthase protein,” or a “Non-Muscle Adenylosuccinate Synthase polypeptide” is identical to the sequence of SEQ ID NO:134. Polypeptides encoded by splice variants of Non-Muscle Adenylosuccinate Synthase nucleic acid sequences, as well as by Non-Muscle Adenylosuccinate Synthase nucleic acids containing a mutation, for example, a mutation resulting from the insertion of a virus in the Non-Muscle Adenylosuccinate Synthase nucleic acid sequence of SEQ ID NO:133 between nucleotides 217 and 218, or at nucleotide 209, are also included in this definition. A “Non-Muscle Adenylosuccinate Synthase protein” or a “Non-Muscle Adenylosuccinate Synthase polypeptide,” as referred to herein, plays a role in head and eye development and cell death regulation. For example, in zebrafish, the loss of a Non-Muscle Adenylosuccinate Synthase polypeptide in a cell results in mutants that display a head and eyes that are at least 50% smaller than those of age-matched wild-type zebrafish. In addition, these mutants have some apoptotic. Cells and lack jaws and branchial arches. Accordingly, a “Non-Muscle Adenylosuccinate Synthase protein” or a “Non-Muscle Adenylosuccinate Synthase polypeptide” may be used as a marker for, or to prevent or treat, for example, a disorder associated with microcephaly, such as Angelman's syndrome or cri du chat, or a neurodegenerative disease characterized by excess cell death, such as Alzheimer's disease, Parkinson's disease, or spinocerebellar ataxia, or a cartilage or connective tissue disorder, such as arthritis or juvenile rheumatoid arthritis, or a craniofacial defect characterized by an underdeveloped jaw, such as Treacher-Collins syndrome or Marfan's syndrome.

By a “Nuclear Cap Binding Protein Subunit 2 protein,” or a “Nuclear Cap Binding Protein Subunit 2 polypeptide” is meant a polypeptide that has at least 85% amino acid sequence identity to the zebrafish Nuclear Cap Binding Protein Subunit 2 amino acid sequence of SEQ ID NO:136 over a region spanning at least 143 contiguous amino acids. Desirably, a “Nuclear Cap Binding Protein Subunit 2 Protein” or a “Nuclear Cap Binding Protein Subunit 2 polypeptide” is at least 90%, 95%, or 98% identical to the sequence of SEQ ID NO:136 over at least 75, 100, or 135 contiguous amino acids. In a more desirable embodiment, a “Nuclear Cap Binding Protein Subunit 2 protein,” or a “Nuclear Cap Binding Protein Subunit 2 polypeptide” is identical to the sequence of SEQ ID NO:136. Polypeptides encoded by splice variants of Nuclear Cap Binding Protein Subunit 2 nucleic acid sequences, as well as by Nuclear Cap Binding. Protein Subunit 2 nucleic acids containing a mutation, for example, a mutation resulting from the insertion of a virus in the Nuclear Cap Binding Protein Subunit 2 nucleic acid sequence of SEQ ID NO:135 between nucleotides 137 and 138, or at nucleotide 209, are also included in this definition. A “Nuclear Cap Binding Protein Subunit 2 protein” or a “Nuclear Cap Binding Protein Subunit 2 polypeptide,” as referred to herein, plays a role in head and eye development. For example, in zebrafish, the loss of a Nuclear Cap Binding Protein Subunit 2 polypeptide in a cell results in mutants that display a head and eyes that are at least 25% smaller than those of age-matched wild-type zebrafish by day four of development. In addition, these mutants have transient necrosis in the CNS between 24 and 48 hours of development, underdeveloped jaws, an underdeveloped stomach, and lack branchial arches three and four. Accordingly, a “Nuclear Cap Binding Protein Subunit 2 protein” or a “Nuclear Cap Binding Protein Subunit 2 polypeptide” may be used as a marker for, or to prevent or treat, for example, a disorder associated with microcephaly, such as Angelman's syndrome or cri du chat, or a craniofacial defect, such as Apert, Crouzon, Pfeiffer and Saethre-Chotzen Syndromes, or a necrotizing brain disorder, such as Leigh's disease, or subacute necrotizing encephalomyelopathy, or a degenerative eye disease, such as retinitis pigmentosa, macular degeneration, Friedreich's ataxia, or Laurence-Moon syndrome, or a cartilage or connective tissue disease, such as arthritis.

By an “Ornithine Decarboxylase protein,” or an “Ornithine Decarboxylase polypeptide” is meant a polypeptide encoded by a Ornithine Decarboxylase gene that, desirably, is identical to the zebrafish Ornithine Decarboxylase amino acid sequence of SEQ ID NO:138. Polypeptides encoded by splice variants of Ornithine Decarboxylase nucleic acid sequences, as well as by Ornithine Decarboxylase nucleic acids containing a mutation, for example, a mutation resulting from the insertion of a virus in the Ornithine Decarboxylase nucleic acid sequence of SEQ ID NO:137 between nucleotides 97 and 98 are also included in this definition. An “Ornithine Decarboxylase protein” or an “Ornithine Decarboxylase polypeptide,” as referred to herein, plays a role in head development and necrosis. For example, in zebrafish, the loss of an Ornithine Decarboxylase polypeptide in a cell results in mutants that display display a head and eyes that are at least 33% smaller than those of age-matched wild-type zebrafish. In addition, these mutants have underdeveloped jaws and branchial arches relative to wild-type zebrafish. Accordingly, an “Ornithine Decarboxylase protein” or an “Ornithine Decarboxylase polypeptide” may be used as a marker for, or to prevent or treat, for example, a craniofacial defect, such as Apert, Crouzon, Pfeiffer or Saethre-Chotzen Syndromes, or a disorder associated with microcephaly, such as Angelman's syndrome or cri du chat, or an eye malformation syndrome, such as oculorenal syndrome, or Reiger syndrome, or a cartilage or connective tissue disorder, such as arthritis.

By a “Protein Phosphatase 1 Nuclear Targeting Subunit protein (PNUTS),” or a “Protein Phosphatase 1 Nuclear Targeting Subunit (PNUTS) polypeptide” is meant a polypeptide that has at least 55% amino acid sequence identity to the zebrafish Protein Phosphatase 1 Nuclear Targeting Subunit amino acid sequence of SEQ ID NO:140 over a region spanning at least 636 contiguous amino acids. Desirably, a “Protein Phosphatase 1 Nuclear Targeting Subunit protein” or a “Protein Phosphatase 1 Nuclear Targeting Subunit polypeptide” is at least 60%, 70%, 80%, 90%, 95%, or 98% identical to the sequence of SEQ ID NO:140. In a more desirable embodiment, a “Protein Phosphatase 1 Nuclear Targeting Subunit protein (PNUTS),” or a “Protein Phosphatase 1 Nuclear Targeting Subunit (PNUTS) polypeptide” is identical to the sequence of SEQ ID NO:140 over at least 400, 500, 550, 600, or 636 contiguous amino acids. Polypeptides encoded by splice variants of Protein Phosphatase 1 Nuclear Targeting Subunit nucleic acid sequences, as well as by Protein Phosphatase 1 Nuclear Targeting Subunit nucleic acids containing a mutation, for example, a mutation resulting from the insertion of a virus in the Protein Phosphatase 1 Nuclear Targeting Subunit nucleic acid sequence of SEQ ID NO:139 at nucleotide 303 are also included in this definition. A “Protein Phosphatase 1 Nuclear Targeting Subunit protein” or a “Protein Phosphatase 1 Nuclear Targeting Subunit polypeptide,” as referred to herein, plays a role in head development. For example, in zebrafish, the loss of a Protein Phosphatase 1 Nuclear Targeting Subunit polypeptide in a cell results in mutants having slightly smaller head than those of age-matched wild-type zebrafish. In addition, these mutants have slightly compressed jaws and an underdeveloped gut relative to wild-type zebrafish. Accordingly, a “Protein Phosphatase 1 Nuclear Targeting Subunit protein” or a “Protein Phosphatase 1 Nuclear Targeting Subunit polypeptide” may be used as a marker for, or to prevent or treat, for example, craniofacial defects characterized by an underdeveloped jaw, such as Treacher-Collins syndrome or Marfan's syndrome.

By a “Mitochondrial Inner Membrane Translocating Protein (rTIM23),” or a “Mitochondrial Inner Membrane Translocating (rTIM23) polypeptide” is meant a polypeptide that has at least 79% amino acid sequence identity to the zebrafish Mitochondrial Inner Membrane Translocating Protein (rTIM23) amino acid sequence of SEQ ID NO:142 over at least 75, 100, 125, 150, 175, or 190 contiguous amino acids. Desirably, a “Mitochondrial Inner Membrane Translocating Protein” or a “Mitochondrial Inner Membrane Translocating Protein (rTIM23) polypeptide” is at least 79%, 80%, or 90% identical to the sequence of SEQ ID NO:142. In a more desirable embodiment, a “Mitochondrial Inner Membrane Translocating Protein (rTIM23),” or a “Mitochondrial Inner Membrane Translocating (rTIM23) polypeptide” is identical to the sequence of SEQ ID NO:142. Polypeptides encoded by splice variants of Mitochondrial Inner Membrane Translocating Protein (rTIM23) nucleic acid sequences, as well as by Mitochondrial Inner Membrane Translocating Protein (rTIM23) nucleic acids containing a mutation, for example, a mutation resulting from the insertion of a virus in the Mitochondrial Inner Membrane Translocating Protein (rTIM23) nucleic acid sequence of SEQ ID NO:141 at nucleotide 100 are also included in this definition. A “Mitochondrial Inner Membrane Translocating Protein” or a “Mitochondrial Inner Membrane Translocating Protein (rTIM23) polypeptide,” as referred to herein, plays a role in development of the circulatory system. For example, in zebrafish, the loss of a Mitochondrial Inner Membrane Translocating polypeptide in a cell results in mutants that display display lighter eyes than wild-type zebrafish and defects in the tail blood vessel. In addition, most of these mutants are dying by day four of development. Accordingly, a “Mitochondrial Inner Membrane Translocating Protein” or a “Mitochondrial Inner Membrane Translocating polypeptide” may be used as a marker for, or to prevent or treat, for example, a pigmentation disorder, such as retinitis pigmentosa, human pigmentary glaucoma, Wagner's disease, Cockayne's syndrome, Kearns-Sayre disease, Waardenburg syndrome, Usher's syndrome, or Multiple lentigines syndrome, or a circulatory disorder, such as stroke.

By a “1447 protein,” or a “1447 polypeptide” is meant a polypeptide that has at least 59% amino acid sequence identity to the zebrafish 1447 amino acid sequence of SEQ ID NO:144 over a region spanning at least 738 contiguous amino acids. Desirably, a “1447 protein” or a “1447 polypeptide” is at least 70%, 80%, 90%, 95%, or 98% identical to the sequence of SEQ ID NO:144 over at least 500, 550, 600, 650, 700, or 738 contiguous amino acids. In a more desirable embodiment, a “1447 protein,” or a “1447 polypeptide” is identical to the sequence of SEQ ID NO:144. Polypeptides encoded by splice variants of 1447 nucleic acid sequences, as well as by 1447 nucleic acids containing a mutation, for example, a mutation resulting from the insertion of a virus in the 1447 nucleic acid sequence of SEQ ID NO:143 between nucleotides 227 and 228 are also included in this definition. A “1447 protein” or a “1447 polypeptide,” as referred to herein, plays a role in development of the head, eyes, and jaw. For example, in zebrafish, the loss of a 1447 polypeptide in a cell results in mutants that display head and eyes that are at least 25% smaller than those of three day-old wild-type zebrafish. In addition, these mutants lack, or have severely reduced, mandibular or branchial arches. Furthermore, these mutants have shorter fins, an underdeveloped stomach, lack a pancreas, and have bent tails. Accordingly, a “1447 protein” or a “1447 polypeptide” may be used as a marker for, or to prevent or treat, for example, a pancreatic disorder, such as diabetes, or a limb formation defect, such as achondroplasia, Ellis-van Creveld syndrome, Poland sequence, or Pfeiffer syndrome, or a cartilage or connective tissue disease, such as arthritis, or a disorder associated with microcephaly, such as Angelman's syndrome or cri du chat.

By an “ARS2 protein,” or an “ARS2 polypeptide” is meant a polypeptide that has at least 69% amino acid sequence identity to the zebrafish ARS2 amino acid sequence of SEQ ID NO:146 over a region spanning at least 917 contiguous amino acids. Desirably, an “ARS2 protein” or an “ARS2 polypeptide” is at least 79%, 80%, 90%, 95%, or 98% identical to the sequence of SEQ ID NO:146 over at least 700, 750, 800, 850, or 917 contiguous amino acids. In a more desirable embodiment, an “ARS2 protein,” or an “ARS2 polypeptide” is identical to the sequence of SEQ ID NO:146. Polypeptides encoded by splice variants of ARS2 nucleic acid sequences, as well as by ARS2 nucleic acids containing a mutation, for example, a mutation resulting from the insertion of a virus in the ARS2 nucleic acid sequence of SEQ ID NO:145 between nucleotides 103 and 104 are also included in this definition. An “ARS2 protein” or an “ARS2 polypeptide,” as referred to herein, plays a role jaw development and pigmentation. For example, in zebrafish, the loss of an ARS2 polypeptide in a cell results in mutants that display underdeveloped jaws and have necrosis in the tectum. In addition, these mutants have flecks of pigment in the otholiths and widespread edema by day five of development. Accordingly, an “ARS2 protein” or an “ARS2 polypeptide” may be used as a marker for, or to prevent or treat, such as a pigmentation disorder, such as retinitis pigmentosa, human pigmentary glaucoma, Wagner's disease, Cockayne's syndrome, Kearns-Sayre disease, Waardenburg syndrome, Usher's syndrome, or multiple lentigines syndrome, or craniofacial defects characterized by an underdeveloped jaw, such as Treacher-Collins syndrome or Marfan's syndrome.

By a “Sec61 Alpha protein,” or a “Sec61 Alpha polypeptide” is meant a polypeptide that, desirably, is identical to the zebrafish Sec61 Alpha amino acid sequence of SEQ ID NO:148 over a region spanning at least 190 contiguous amino acids. However, polypeptides encoded by splice variants of Sec61 Alpha nucleic acid sequences, as well as by Sec61 Alpha nucleic acids containing a mutation, for example, a mutation resulting from the insertion of a virus in the Sec61 Alpha nucleic acid sequence of SEQ ID NO:147 between nucleotides 132 and 133 are also included in this definition. A “Sec61 Alpha protein” or a “Sec61 Alpha polypeptide,” as referred to herein, plays a role head, eye and body development. For example, in zebrafish, the loss of a Sec61 Alpha polypeptide in a cell results in mutants that display a bent body, head and eyes that are at least 25% smaller than those of age-matched wild-type zebrafish, and having a lack of development of the jaw or branchial arches. Accordingly, a “Sec61 Alpha protein” or a “Sec61 Alpha polypeptide” may be used as a marker for, or to prevent or treat, craniofacial defects characterized by an underdeveloped jaw, such as Treacher-Collins syndrome or Marfan's syndrome, or a disorder associated with microcephaly, such as Angelman's syndrome or cri du chat, or an eye malformation syndrome, such as oculorenal syndrome, or Reiger syndrome.

By a “BAF53a protein,” or a “BAF53a polypeptide” is meant a polypeptide that has at least 88% amino acid sequence identity to the zebrafish BAF53a amino acid sequence of SEQ ID NO:150 over a region spanning at least 429 contiguous amino acids. Desirably, a “BAF53a protein” or a “BAF53a polypeptide” is at least 90%, 95%, or 99% identical to the sequence of SEQ ID NO:150 over a region spanning at least 300, 350, 400, or 429 contiguous amino acids. In a more desirable embodiment, a “BAF53a protein,” or a “BAF53a polypeptide” is identical to the sequence of SEQ ID NO:150. Polypeptides encoded by splice variants of BAF53a nucleic acid sequences, as well as by BAF53A nucleic acids containing a mutation, for example, a mutation resulting from the insertion of a virus in the BAF53A nucleic acid sequence of SEQ ID NO:149 at nucleotide 160 are also included in this definition. A “BAF53a protein” or a “BAF53A polypeptide,” as referred to herein, plays a role body, eye, and brain development. For example, in zebrafish, the loss of a BAF53a polypeptide in a cell results in fish that display a curved body, small underdeveloped eyes, and enlarged ventricles. Accordingly, a “BAF53a protein” or a “BAF53a polypeptide” may be used as a marker for, or to prevent or treat, for example, an eye malformation syndrome, such as oculorenal syndrome, or Reiger syndrome, or a congenital brain defect that causes mental retardation, such as microencephaly, anencephaly, Down's syndrome, Chiari Malformation, Colpocephaly, holoprosencephaly, lissencephaly, or megalencephaly.

By a “Histone Deacetylase protein,” or a “Histone Deacetylase polypeptide” is meant a polypeptide that has at least 90% amino acid sequence identity to the zebrafish Histone Deacetylase amino acid sequence of SEQ ID NO:152 over a region spanning at least 483 contiguous amino acids. Desirably, a “Histone Deacetylase protein” or a “Histone Deacetylase polypeptide” is at least 93%, 95%, or 98% identical to the sequence of SEQ ID NO:152 over at least 300, 350, 400, 450, or 483 contiguous amino acids. In a more desirable embodiment, a “Histone Deacetylase protein” or a “Histone Deacetylase polypeptide” is identical to the sequence of SEQ ID NO:152. Polypeptides encoded by splice variants of Histone Deacetylase nucleic acid sequences, as well as by Histone Deacetylase nucleic acids containing a mutation, for example, a mutation resulting from the insertion of a virus in the Histone Deacetylase nucleic acid sequence of SEQ ID NO: 151 between nucleotides 98 and 99, or at nucleotide 88, are also included in this definition. A “Histone Deacetylase protein” or a “Histone Deacetylase polypeptide,” as referred to herein, plays a role in the development of the heart, eyes, semicircular canals, otoliths, and cartilaginous structures. For example, in zebrafish, the loss of a Histone Deacetylase polypeptide in a cell results in mutants that display enlarged hearts, with atria twice the size of wild-type, eyes that are 33% smaller than wild-type, and ears that lack semicircular canals and have close together or fused otoliths. In addition, fin buds, jaws and branchial arches all fail to develop. Moreover, at day five of development, Alcian blue fails to stain any cartilage corresponding to the pectoral fins, jaw, branchial arches, or the neurocranium. Accordingly, a “Histone Deacetylase protein” or a “Histone Deacetylase polypeptide” may be used as a marker for, or to prevent or treat, for example, hearing disorders, such as Usher syndrome, Waardenburg syndrome, or other disorders characterized by congenital deafness; or a cartilage, connective tissue, or bone disorder, such as arthritis, or a limb formation defect, such as achondroplasia, Ellis-van Creveld syndrome, Poland sequence, or Pfeiffer syndrome, or a congenital heart defect, such as an atrial or ventricular septal defect, or an eye malformation syndrome, such as oculorenal syndrome, or Reiger syndrome.

By a “Fibroblast Isoform of the ADP/ATP Carrier protein,” or a “Fibroblast Isoform of the ADP/ATP Carrier polypeptide” is meant a polypeptide that has at least 93% amino acid sequence identity to the zebrafish Fibroblast Isoform of the Fibroblast Isoform of the ADP/ATP Carrier Protein amino acid sequence of SEQ ID NO:154 over a region spanning at least 298 contiguous amino acids. Desirably, a “Fibroblast Isoform of the ADP/ATP Carrier Protein protein” or a “Fibroblast Isoform of the ADP/ATP Carrier Protein polypeptide” is at least 93% or 95% identical to to the sequence of SEQ ID NO:154 over a region spanning at least 175, 200, 225, 250, 275, or 298 contiguous amino acids. In more desirable embodiments, a “Fibroblast Isoform of the ADP/ATP Carrier protein,” or a “Fibroblast Isoform of the ADP/ATP Carrier polypeptide” is identical to the sequence of SEQ ID NO:154. Polypeptides encoded by splice variants of Fibroblast Isoform of the ADP/ATP Carrier Protein nucleic acid sequences, as well as by Fibroblast Isoform of the ADP/ATP Carrier Protein nucleic acids containing a mutation, for example, a mutation resulting from the insertion of a virus in the Fibroblast Isoform of the ADP/ATP Carrier Protein nucleic acid sequence of SEQ ID NO:153 between nucleotides 178 and 179 are also included in this definition. A “Fibroblast Isoform of the ADP/ATP Carrier Protein protein” or a “Fibroblast Isoform of the ADP/ATP Carrier Protein polypeptide,” as referred to herein, plays a role in lung or, in zebrafish, swim bladder development. For example, in zebrafish, the loss of a Fibroblast Isoform of the ADP/ATP Carrier Protein polypeptide in a cell results in mutants that display swim bladders that fail to inflate. Accordingly, a “Fibroblast Isoform of the ADP/ATP Carrier Protein protein” or a “Fibroblast Isoform of the ADP/ATP Carrier Protein polypeptide” may be used as a marker for, or to prevent or treat, for example, a pulmonary disease, such as asthma.

By a “TAFII-55 protein,” or a “TAFII-55 polypeptide” is meant a polypeptide that has at least 68% amino acid sequence identity to the zebrafish TAFII-55 amino acid sequence of SEQ ID NO:156 over a region spanning at least 336 contiguous amino acids. Desirably, a “TAFII-55 protein” or a “TAFII-5 polypeptide” is at least 80%, 90%, 95%, or 98% identical to the sequence of SEQ ID NO:156 over a region spanning at least 336 contiguous amino acids. In a desirable embodiment, a “TAFII-55 protein,” or a “TAFII-55 polypeptide” is identical to the sequence of SEQ ID NO:156. Polypeptides encoded by splice variants of TAFII-55 nucleic acid sequences, as well as by TAFII-55 nucleic acids containing a mutation, for example, a mutation resulting from the insertion of a virus in the TAFII-55 nucleic acid sequence of SEQ ID NO:155 between nucleotides 107 and 108 are also included in this definition. A “TAFII-55 protein” or a “TAFII-55 polypeptide,” as referred to herein, plays a role in head, eye, lung, or, in zebrafish, swim bladder development. For example, in zebrafish, the loss of a TAFII-55 polypeptide in a cell results in mutants that display swim bladders that fail to fill. In addition the mutants have heads that are approximately 20% smaller, and eyes that are approximately 33% smaller than those of age-matched wild-type zebrafish. Accordingly, a “TAFII-55 protein” or a “TAFII-55 polypeptide” may be used as a marker for, or to prevent or treat, for example, a disorder associated with microcephaly, such as Angelman's syndrome or cri du chat, or a pulmonary disease, such as asthma, or an eye malformation syndrome, such as oculorenal syndrome, or Reiger syndrome.

By a “904 nucleic acid sequence” is meant a nucleic acid molecule that is at least 79%, 85%, 90%, 95%, or 98% identical, or to the sequence of SEQ ID NO:1. In a desirable embodiment, a “904 nucleic acid sequence” is identical to the sequence of SEQ ID NO:1. Such nucleic acid molecules include genomic DNA, cDNA, or RNA (e.g., mRNA) molecule, that encodes a 904 polypeptide (e.g., SEQ ID NO:2) or a portion thereof, as defined above. A mutation in a 904 nucleic acid molecule can be characterized, for example, by the insertion of a retrovirus into the nucleic acid sequence, for example, at nucleotide 1315 of SEQ ID NO:1, using methods described herein. In addition, the invention includes mutations that result in aberrant 904 expression or function, including, as examples, null mutations and mutations causing truncations.

By a “POU2 nucleic acid sequence” is meant a nucleic acid sequence that is identical to the zebrafish POU2 nucleic acid sequence of SEQ ID NO:3. However, nucleic acid molecules consisting of splice variants of POU2 nucleic acid sequences, as well as POU2 nucleic acid sequences containing a mutation, for example, a mutation resulting from the insertion of a virus, e.g., an insertion at nucleotide 653 or 1088 of SEQ ID NO:3, are also included in this definition. By a “POU2 nucleic acid sequence” is also meant a nucleic acid molecule, such as a genomic DNA, cDNA, or RNA (e.g., mRNA) molecule, that encodes a POU2 polypeptide or a zebrafish (SEQ ID NO:4,) POU2 polypeptide, or a portion thereof, as defined above. A mutation in a POU2 nucleic acid molecule can be characterized, for example, by the insertion of a retrovirus into the nucleic acid sequence, for example, using methods described herein. In addition, the invention includes mutations that result in aberrant POU2 expression or function, including, as examples, null mutations and mutations causing truncations.

By a “40S Ribosomal Protein S18 nucleic acid sequence” is meant a nucleic acid sequence that has at least 97%, 98%, or 99% identity to the zebrafish 40S Ribosomal Protein S18 nucleic acid sequence of SEQ ID NO:5 over at least 152, 200, 250, 300, 400, or 500 nucleotides. In a desirable embodiment, a “40S Ribosomal Protein S18 nucleic acid sequence” is identical to the sequence of SEQ ID NO:5. However, nucleic acid molecules consisting of splice variants of 40S Ribosomal Protein S18 nucleic acid sequences, as well as 40S Ribosomal Protein S18 nucleic acid sequences containing a mutation, for example, a mutation resulting from the insertion of a virus, e.g., an insertion between nucleotides 220 and 221 of SEQ ID NO:5, are also included in this definition. By a “40S Ribosomal Protein S18 nucleic acid sequence” is also meant a nucleic acid molecule, such as a genomic DNA, cDNA, or RNA (e.g., mRNA) molecule, that encodes a 40S Ribosomal Protein S18 polypeptide or a zebrafish (e.g., SEQ ID NO:6) 40S Ribosomal Protein S18 polypeptide, or a portion thereof, as defined above. A mutation in a 40S Ribosomal Protein S18 nucleic acid molecule can be characterized, for example, by the insertion of a retrovirus into the nucleic acid sequence, for example, using methods described herein. In addition, the invention includes mutations that result in aberrant 40S Ribosomal Protein S18 expression or function, including, as examples, null mutations and mutations causing truncations.

By a “U2AF nucleic acid sequence” is meant a nucleic acid sequence that has at least 82%, 85%, 90%, 95%, or 97% identity to the zebrafish U2AF nucleic acid sequence of SEQ ID NO:7 over at least 100, 200, 300, 400, 537, 750, 1000, or 1500 nucleotides. In a desirable embodiment, a “U2AF nucleic acid sequence” is identical to the sequence of SEQ ID NO:7. Nucleic acid molecules consisting of splice variants of U2AF nucleic acid sequences, as well as U2AF nucleic acid sequences containing a mutation, for example, a mutation resulting from the insertion of a virus, e.g., an insertion between nucleotides 46 and 47 of SEQ ID NO:7, are also included in this definition. By a “U2AF nucleic acid sequence” is also meant a nucleic acid molecule, such as a genomic DNA, cDNA, or RNA (e.g., mRNA) molecule, that encodes a U2AF polypeptide or a zebrafish U2AF polypeptide (e.g., SEQ ID NO:8), or a portion thereof, as defined above. A mutation in a U2AF nucleic acid molecule can be characterized, for example, by the insertion of a retrovirus into the nucleic acid sequence, for example, using methods described herein. In addition, the invention includes mutations that result in aberrant U2AF expression or function, including, as examples, null mutations and mutations causing truncations.

By a “954 nucleic acid sequence” is meant a nucleic acid sequence that has at least 40%, 45%, 50%, 55%, 60%, 70%, 80%, 90%, 95%, or 98% identity to the zebrafish 954 nucleic acid sequence of SEQ ID NO:9 over at least 100, 200, 250, 300, 400, 500, 600, 750, 1000, 1250, 1500, 1750, 2000, or 2100 contiguous nucleotides. In a desirable embodiment, a “954 nucleic acid sequence” is identical to the sequence of SEQ ID NO:9. Nucleic acid molecules consisting of splice variants of 954 nucleic acid sequences, as well as 954 nucleic acid sequences containing a mutation, for example, a mutation resulting from the insertion of a virus, e.g., an insertion at nucleotide 432 or 506 of SEQ ID NO:9, are also included in this definition. By a “954 nucleic acid sequence” is also meant a nucleic acid molecule, such as a genomic DNA, cDNA, or RNA (e.g., mRNA) molecule, that encodes a 954 polypeptide or a zebrafish 954 polypeptide (e.g., SEQ ID NO:10), or a portion thereof, as defined above. A mutation in a 954 nucleic acid molecule can be characterized, for example, by the insertion of a retrovirus into the nucleic acid sequence, for example, using methods described herein. In addition, the invention includes mutations that result in aberrant 954 expression or function, including, only as examples, null mutations and mutations causing truncations.

By a “Neurogenin Related Protein-1 nucleic acid sequence” or a “Nrp-1 nucleic acid sequence” is meant a nucleic acid sequence that identical to a zebrafish Nrp-1 nucleic acid sequence, for example, that of SEQ ID NO:11. Nucleic acid molecules consisting of splice variants of Nrp-1 nucleic acid sequences, as well as Nrp-1 nucleic acid sequences containing a mutation, for example, a mutation resulting from the insertion of a virus, e.g., an insertion at nucleotide 1149 of SEQ ID NO:11, are also included in this definition. By a “Nrp-1 nucleic acid sequence” is also meant a nucleic acid molecule, such as a genomic DNA, cDNA, or RNA (e.g., mRNA) molecule, that encodes a Nrp-1 polypeptide or a zebrafish Nrp-1 polypeptide (e.g., SEQ ID NO:12), or a portion thereof, as defined above. A mutation in a Nrp-1 nucleic acid molecule can be characterized, for example, by the insertion of a retrovirus into the nucleic acid sequence, for example, using methods described herein. In addition, the invention includes mutations that result in aberrant Nrp-1 expression or function, including, only as examples, null mutations and mutations causing truncations.

By a “Caudal nucleic acid sequence” or “Cad-1 nucleic acid sequence” is meant a nucleic acid sequence that is identical to a zebrafish Caudal or Cad-1 nucleic acid sequence, for example, that of SEQ ID NO:13. Nucleic acid molecules consisting of splice variants of Cad-1 nucleic acid sequences, as well as Cad-1 nucleic acid sequences containing a mutation, for example, a mutation resulting from the insertion of a virus, e.g., an insertion between nucleotides 583 and 584 of SEQ ID NO:13, are also included in this definition. By a “Caudal nucleic acid sequence” or “Cad-1 nucleic acid sequence” is also meant a nucleic acid molecule, such as a genomic DNA, cDNA, or RNA (e.g., mRNA) molecule, that encodes a Caudal or Cad-1 polypeptide or a zebrafish Cad-1 polypeptide (e.g., SEQ ID NO:14), or a portion thereof, as defined above. A mutation in a cad-1 nucleic acid molecule can be characterized, for example, by the insertion of a retrovirus into the nucleic acid sequence, for example, using methods described herein. In addition, the invention includes mutations that result in aberrant Caudal or Cad-1 expression or function, including, only as examples, null mutations and mutations causing truncations.

By a “V-ATPase Alpha Subunit nucleic acid sequence” is meant a nucleic acid sequence that has at least 85%, 90%, 95%, or 98% identity to the zebrafish V-ATPase Alpha Subunit nucleic acid sequence of SEQ ID NO:15 over at least 800, 1000, or 1200 nucleotides. In a desirable embodiment, a “V-ATPase Alpha Subunit nucleic acid sequence” is identical to the sequence of SEQ ID NO:15. Nucleic acid molecules consisting of splice variants of V-ATPase Alpha Subunit nucleic acid sequences, as well as V-ATPase Alpha Subunit nucleic acid sequences containing a mutation, for example, a mutation resulting from the insertion of a virus, e.g., an insertion at nucleotide 169 of SEQ ID NO:15, are also included in this definition. By a “V-ATPase Alpha Subunit nucleic acid sequence” is also meant a nucleic acid molecule, such as a genomic DNA, cDNA, or RNA (e.g., mRNA) molecule, that encodes a V-ATPase Alpha Subunit polypeptide or a zebrafish V-ATPase Alpha Subunit polypeptide (e.g., SEQ ID NO:16), or a portion thereof, as defined above. A mutation in a V-ATPase Alpha Subunit nucleic acid molecule can be characterized, for example, by the insertion of a retrovirus into the nucleic acid sequence, for example, using methods described herein. In addition, the invention includes mutations that result in aberrant V-ATPase Alpha Subunit expression or function, including, only as examples, null mutations and mutations causing truncations.

By a “V-ATPase SFD Subunit nucleic acid sequence” is meant a nucleic acid sequence that has at least 81%, 85%, 90%, 95%, or 98% identity to the zebrafish V-ATPase SFD Subunit nucleic acid sequence of SEQ ID NO:17 over at least 100, 200, 300, 400, 537, 750, 1000, or 1500 nucleotides. In a desirable embodiment, a “V-ATPase SFD Subunit nucleic acid sequence” is identical to the sequence of SEQ ID NO:17. Nucleic acid molecules consisting of splice variants of V-ATPase SFD Subunit nucleic acid sequences, as well as V-ATPase SFD Subunit nucleic acid sequences containing a mutation, for example, a mutation resulting from the insertion of a virus, e.g., an insertion between nucleotides 31 and 32 of SEQ ID NO:17, are also included in this definition. By a “V-ATPase SFD Subunit nucleic acid sequence” is also meant a nucleic acid molecule, such as a genomic DNA, cDNA, or RNA (e.g., mRNA) molecule, that encodes a V-ATPase SFD subunit polypeptide or a zebrafish V-ATPase SFD Subunit polypeptide (e.g., SEQ ID NO:18), or a portion thereof, as defined above. A mutation in a V-ATPase SFD Subunit nucleic acid molecule can be characterized, for example, by the insertion of a retrovirus into the nucleic acid sequence, for example, using methods described herein. In addition, the invention includes mutations that result in aberrant V-ATPase SFD Subunit expression or function, including, as examples, null mutations and mutations causing truncations.

By a “1463 nucleic acid sequence” is meant a nucleic acid sequence that has at least 74%, 80%, 85%, 90%, 95%, or 98% identity to the zebrafish 1463 nucleic acid sequence of SEQ ID NO:157 over at least 175, 200, 300, 400, 500, 750, 1000, 1500, 2000, 2500, or 3000 nucleotides. In a desirable embodiment, a “1463” nucleic acid sequence” is identical to the sequence of SEQ ID NO:157. Nucleic acid molecules consisting of splice variants of 1463 nucleic acid sequences, as well as 1463 nucleic acid sequences containing a mutation, for example, a mutation resulting from the insertion of a virus, e.g., an insertion between nucleotides 389 and 390 of SEQ ID NO:157, are also included in this definition. By a “1463 nucleic acid sequence” is also meant a nucleic acid molecule, such as a genomic DNA, cDNA, or RNA (e.g., mRNA) molecule, that encodes a 1463 polypeptide or a zebrafish 1463 polypeptide (e.g., SEQ ID NO:158), or a portion thereof, as defined above. A mutation in a 1463 nucleic acid molecule can be characterized, for example, by the insertion of a retrovirus into the nucleic acid sequence, for example, using methods described herein. In addition, the invention includes mutations that result in aberrant 1463 expression or function, including, as examples, null mutations and mutations causing truncations.

By a “VPSP18 nucleic acid sequence” is meant a nucleic acid sequence that has at least 83%, 90%, 95%, or 98% identity to the zebrafish VPSP18 nucleic acid sequence of SEQ ID NO:21 spanning at least 50, 70, 100, 200, 300, 500, 750, 1000, 1200, or 1400 contiguous nucleic acids. In a desirable embodiment, “VPSP18 nucleic acid sequence” is identical to the sequence of SEQ ID NO:21. Nucleic acid molecules consisting of splice variants of VPSP18 nucleic acid sequences, as well as VPSP18 nucleic acid sequences containing a mutation, for example, a mutation resulting from the insertion of a virus, e.g., an insertion at nucleotide 2336 of SEQ ID NO:21, are also included in this definition. A mutation in a VPSP18 nucleic acid molecule can be characterized, for example, by the insertion of a retrovirus into the nucleic acid sequence, for example, using methods described herein. In addition, the invention includes mutations that result in aberrant VPSP18 expression or function, including, as examples, null mutations and mutations causing truncations.

By a “Pescadillo nucleic acid sequence” is meant a nucleic acid sequence that is identical to a zebrafish Pescadillo nucleic acid sequence, for example, that of GenBank Accession Number U77627. Nucleic acid molecules encoded by splice variants of Pescadillo nucleic acid sequences, as well as Pescadillo nucleic acid sequences containing a mutation, for example, a mutation resulting from the insertion of a virus, e.g., an insertion at approximately nucleotide 20 of the U77627 sequence, are also included in this definition. By a “Pescadillo nucleic acid sequence” is also meant a nucleic acid molecule, such as a genomic DNA, cDNA, or RNA (e.g., mRNA) molecule, that encodes a Pescadillo polypeptide or a zebrafish Pescadillo polypeptide, or a portion thereof, as defined above. A mutation in a Pescadillo nucleic acid molecule can be characterized, for example, by the insertion of a retrovirus into the nucleic acid sequence, for example, using methods described herein. In addition, the invention includes mutations that result in aberrant Pescadillo expression or function, including, as examples, null mutations and mutations causing truncations.

By a “HNF-β/vHNF1 nucleic acid sequence” is meant a nucleic acid sequence that has at least 81%, 85%, 90%, 95%, or 98% identity to the zebrafish HNF-β/vHNF1 nucleic acid sequence of SEQ ID NO:23 over at least 100, 200, 270, 300, 400, 500, 600, 700, 800, 1000, 1500, 2000, 2500, or 3000 nucleotides. In a desirable embodiment, a “HNF-β/vHNF1 nucleic acid sequence” is identical to the sequence of SEQ ID NO:23. Nucleic acid molecules consisting of splice variants of HNF-β/vHNF1nucleic acid sequences, as well as HNF-β/vHNF1 nucleic acid sequences containing a mutation, for example, a mutation resulting from the insertion of a virus, e.g., an insertion between nucleotides 1682/1683, or at nucleotide 361 or 745 of SEQ ID NO:23, are also included in this definition. By a “HNF-β/vHNF1 nucleic acid sequence” is also meant a nucleic acid molecule, such as a genomic DNA, cDNA, or RNA (e.g., mRNA) molecule, that encodes a HNF-β/vHNF1 polypeptide or a zebrafish HNF-β/vHNF1 polypeptide (e.g., SEQ ID NO:24), or a portion thereof, as defined above. A mutation in a HNF-β/vHNF1 nucleic acid molecule can be characterized, for example, by the insertion of a retrovirus into the nucleic acid sequence, for example, using methods described herein. In addition, the invention includes mutations that result in aberrant HNF-β/vHNF1 expression or function, including, as examples, null mutations and mutations causing truncations.

By a “60S Ribosomal Protein L35 nucleic acid sequence” is meant a nucleic acid sequence that has at least 82%, 85%, 90%, 95%, or 98% identity to the zebrafish 60S Ribosomal Protein L35 nucleic acid sequence of SEQ ID NO:25 over at least 100, 200, 319, 400, ot 450 nucleotides. In a desirable embodiment, a “60S Ribosomal Protein L35 nucleic acid sequence” is identical to the sequence of SEQ ID NO:25. Nucleic acid molecules consisting of splice variants of 60S Ribosomal Protein L35 nucleic acid sequences, as well as 60S Ribosomal Protein L35 nucleic acid sequences containing a mutation, for example, a mutation resulting from the insertion of a virus, e.g., an insertion between nucleotides 30 and 31 of SEQ ID NO:25, are also included in this definition. By a “60S Ribosomal Protein L35 nucleic acid sequence” is also meant a nucleic acid molecule, such as a genomic DNA, cDNA, or RNA (e.g., mRNA) molecule, that encodes a 60S Ribosomal Protein L35 polypeptide or a zebrafish 60S Ribosomal Protein L35 polypeptide (e.g., SEQ ID NO:26), or a portion thereof, as defined above. A mutation in a 60S Ribosomal Protein L35 nucleic acid molecule can be characterized, for example, by the insertion of a retrovirus into the nucleic acid sequence, for example, using methods described herein. In addition, the invention includes mutations that result in aberrant 60S Ribosomal Protein L35 expression or function, including, as examples, null mutations and mutations causing truncations.

By a “60S Ribosomal Protein L44 nucleic acid sequence” is meant a nucleic acid sequence that has at least 85%, 90%, 95%, or 98% identity to the zebrafish 60S Ribosomal Protein L44 nucleic acid sequence of SEQ ID NO:27 over at least 100, 200, 324, or 350 nucleotides. In a desirable embodiment, a “60S Ribosomal Protein L44 nucleic acid sequence” is identical to the sequence of SEQ ID NO:27. Nucleic acid molecules consisting of splice variants of 60S Ribosomal Protein L44 nucleic acid sequences, as well as 60S Ribosomal Protein L44 nucleic acid sequences containing a mutation, for example, a mutation resulting from the insertion of a virus, e.g., an insertion between nucleotides 195 and 196 of the nucleic acid sequence of SEQ ID NO:27, are also included in this definition. By a “60S Ribosomal Protein L44 nucleic acid sequence” is also meant a nucleic acid molecule, such as a genomic DNA, cDNA, or RNA (e.g., mRNA) molecule, that encodes a 60S Ribosomal Protein L44 polypeptide or a zebrafish 60S Ribosomal Protein L44 polypeptide e.g., SEQ ID NO:28, or a portion thereof, as defined above. A mutation in a 60S Ribosomal Protein L44 nucleic acid molecule can be characterized, for example, by the insertion of a retrovirus into the nucleic acid sequence, for example, using methods described herein. In addition, the invention includes mutations that result in aberrant 60S Ribosomal Protein L44 expression or function, including, as examples, null mutations and mutations causing truncations.

By a “CopZ1 nucleic acid sequence” is meant a nucleic acid sequence that is, for example, identical to the zebrafish CopZ1 nucleic acid sequence of SEQ ID NO:29. In a desirable embodiment, a “CopZ1 nucleic acid sequence” is identical to the sequence of SEQ ID NO:29. Nucleic acid molecules consisting of splice variants of CopZ1 nucleic acid sequences, as well as CopZ1 nucleic acid sequences containing a mutation, for example, a mutation resulting from the insertion of a virus, e.g., an insertion between nucleotides 90 and 91 of SEQ ID NO:29, are also included in this definition. By a “CopZ1 nucleic acid sequence” is also meant a nucleic acid molecule, such as a genomic DNA, cDNA, or RNA (e.g., mRNA) molecule, that encodes a CopZ1 polypeptide or a zebrafish CopZ1 polypeptide (e.g., SEQ ID NO:30), or a portion thereof, as defined above. A mutation in a CopZ1 nucleic acid molecule can be characterized, for example, by the insertion of a retrovirus into the nucleic acid sequence, for example, using methods described herein. In addition, the invention includes mutations that result in aberrant CopZ1expression or function, including, as examples, null mutations and mutations causing truncations.

By a “215 nucleic acid sequence” is meant a nucleic acid sequence that has at least 81%, 85%, 90%, 95% or 98% identity to the zebrafish 215 nucleic acid sequence of SEQ ID NO:31 over at least 100, 188, 300, 400, 500, 600, 700, 800, 1000, 1500, or 2000 contiguous nucleotides. In a desirable embodiment, a “215 nucleic acid sequence” is identical to the sequence of SEQ ID NO:31. Nucleic acid molecules consisting of splice variants of 215 nucleic acid sequences, as well as 215 nucleic acid sequences containing a mutation, for example, a mutation resulting from the insertion of a virus, e.g., an insertion between nucleotides 294 and 295 of SEQ ID NO:31, are also included in this definition. By a “215 nucleic acid sequence” is also meant a nucleic acid molecule, such as a genomic DNA, cDNA, or RNA (e.g., mRNA) molecule, that encodes a 215 polypeptide or a zebrafish 215 polypeptide (e.g., SEQ ID NO:32), or a portion thereof, as defined above. A mutation in a 215 nucleic acid molecule can be characterized, for example, by the insertion of a retrovirus into the nucleic acid sequence, for example, using methods described herein. In addition, the invention includes mutations that result in aberrant 215 expression or function, including, as examples, null mutations and mutations causing truncations.

By a “307 nucleic acid sequence” is meant a nucleic acid sequence that has at least 94%, 96%, or 98% identity to the zebrafish 307 nucleic acid sequence of SEQ ID NO:33 over at least 34, 50, 100, 200, 300, 400, 500, 600, 700, 800, or 900 nucleotides. In a desirable embodiment, a “307 nucleic acid sequence” is identical to the sequence of SEQ ID NO:33. Nucleic acid molecules consisting of splice variants of 307 nucleic acid sequences, as well as 307 nucleic acid sequences containing a mutation, for example, a mutation resulting from the insertion of a virus, e.g., an insertion at nucleotide 176 of SEQ ID NO:33, are also included in this definition. By a “307 nucleic acid sequence” is also meant a nucleic acid molecule, such as a genomic DNA, cDNA, or RNA (e.g., mRNA) molecule, that encodes a 307 polypeptide or a zebrafish 307 polypeptide (e.g., SEQ ID NO:34), or a portion thereof, as defined above. A mutation in a 307 nucleic acid molecule can be characterized, for example, by the insertion of a retrovirus into the nucleic acid sequence, for example, using methods described herein. In addition, the invention includes mutations that result in aberrant 307 expression or function, including, as examples, null mutations and mutations causing truncations.

By a “572 nucleic acid sequence” is meant a nucleic acid sequence that has at least 37%, 40%, 50%, 60%, 70%, 80%, 85%, 90%, 95%, 97%, or 99% identity to the zebrafish 572 nucleic acid sequence of SEQ ID NO:35 over at least 50, 100, 150, 200, 250, 300, 350, 400, 500, or 750 contiguous nucleotides. In a desirable embodiment, a “572 nucleic acid sequence” is identical to the sequence of SEQ ID NO:35. Nucleic acid molecules consisting of splice variants of 572 nucleic acid sequences, as well as 572 nucleic acid sequences containing a mutation, for example, a mutation resulting from the insertion of a virus, e.g., an insertion at nucleotide 277 of SEQ ID NO:35, are also included in this definition. By a “572 nucleic acid sequence” is also meant a nucleic acid molecule, such as a genomic DNA, cDNA, or RNA (e.g., mRNA) molecule, that encodes a 572 polypeptide or a zebrafish 572 polypeptide (e.g., SEQ ID NO:36), or a portion thereof, as defined above. A mutation in a 572 nucleic acid molecule can be characterized, for example, by the insertion of a retrovirus into the nucleic acid sequence, for example, using methods described herein. In addition, the invention includes mutations that result in aberrant 572 expression or function, including, as examples, null mutations and mutations causing truncations.

By a “1116A nucleic acid sequence” is meant a nucleic acid sequence that has at least 42%, 45%, 50%, 60%, 70%, 80%, 85%, 90%, or 95% identity to the zebrafish 1116A nucleic acid sequence of SEQ ID NO:37 over at least 1000 nucleotides. In a desirable embodiment, a “1116A nucleic acid sequence” is identical to the sequence of SEQ ID NO:37. Nucleic acid molecules consisting of splice variants of 1116A nucleic acid sequences, as well as 1116A nucleic acid sequences containing a mutation, for example, a mutation resulting from the insertion of a virus, e.g., an insertion at nucleotide 135 of SEQ ID NO:37, are also included in this definition. By a “1116A nucleic acid sequence” is also meant a nucleic acid molecule, such as a genomic DNA, cDNA, or RNA (e.g., mRNA) molecule, that encodes a 1116A polypeptide or a zebrafish 1116A polypeptide (e.g., SEQ ID NO:38), or a portion thereof, as defined above. A mutation in a 1116A nucleic acid molecule can be characterized, for example, by the insertion of a retrovirus into the nucleic acid sequence, for example, using methods described herein. In addition, the invention includes mutations that result in aberrant 1116A expression or function, including, as examples, null mutations and mutations causing truncations.

By a “1548 nucleic acid sequence” is meant a nucleic acid sequence that has at least 78%, 85%, 90%, 95%, or 98% identity to the zebrafish 1548 nucleic acid sequence of SEQ ID NO:39 over at least 100, 200, 300, 400, 503, 600, 700, 800, 900, 1000, 1500, 2000, 2500, or 2900 contiguous nucleotides. In a desirable embodiment, a “1548 nucleic acid sequence” is identical to the sequence of SEQ ID NO:39. Nucleic acid molecules consisting of splice variants of 1548 nucleic acid sequences, as well as 1548 nucleic acid sequences containing a mutation, for example, a mutation resulting from the insertion of a virus, e.g., an insertion at nucleotide 85 of SEQ ID NO:39, are also included in this definition. By a “1548 nucleic acid sequence” is also meant a nucleic acid molecule, such as a genomic DNA, cDNA, or RNA (e.g., mRNA) molecule, that encodes a 1548 polypeptide or a zebrafish 1548 polypeptide (e.g., SEQ ID NO:39), or a portion thereof, as defined above. A mutation in a 1548 nucleic acid molecule can be characterized, for example, by the insertion of a retrovirus into the nucleic acid sequence, for example, using methods described herein. In addition, the invention includes mutations that result in aberrant 1548 expression or function, including, as examples, null mutations and mutations causing truncations.

By a “Casein Kinase 1 α nucleic acid sequence” is meant a nucleic acid sequence that has at least 84%, 90%, 95%, or 98% identity to the zebrafish Casein Kinase 1α nucleic acid sequence of SEQ ID NO:41 over at least 250, 500, 750, 976, 1000, 1250, 1500, or 2000 nucleotides. In a desirable embodiment, a “Casein Kinase 1α nucleic acid sequence” is identical to the sequence of SEQ ID NO:41. Nucleic acid molecules consisting of splice variants of Casein Kinase 1α nucleic acid sequences, as well as Casein Kinase 1α nucleic acid sequences containing a mutation, for example, a mutation resulting from the insertion of a virus, e.g., an insertion between nucleotides 730 and 731 of SEQ ID NO:41, are also included in this definition. By a “Casein Kinase 1α nucleic acid sequence” is also meant a nucleic acid molecule, such as a genomic DNA, cDNA, or RNA (e.g., mRNA) molecule, that encodes a Casein Kinase 1α polypeptide or a zebrafish Casein Kinase 1α polypeptide (e.g., SEQ ID NO:41), or a portion thereof, as defined above. A mutation in a Casein Kinase 1α nucleic acid molecule can be characterized, for example, by the insertion of a retrovirus into the nucleic acid sequence, for example, using methods described herein. In addition, the invention includes mutations that result in aberrant Casein Kinase 1α expression or function, including, as examples, null mutations and mutations causing truncations.

By a “Nodal-Related (squint) nucleic acid sequence” is meant a nucleic acid sequence that is identical to a zebrafish Nodal-Related (squint) nucleic acid sequence, for example, that of SEQ ID NO:43. Nucleic acid molecules consisting of splice variants of Nodal-Related (squint) nucleic acid sequences, as well as Nodal-Related (squint) nucleic acid sequences containing a mutation, for example, a mutation resulting from the insertion of a virus, e.g., an insertion between nucleotides 654 of SEQ ID NO:43, are also included in this definition. By a “Nodal-Related (squint) nucleic acid sequence” is also meant a nucleic acid molecule, such as a genomic DNA, cDNA, or RNA (e.g., mRNA) molecule, that encodes a Nodal-Related (Squint) polypeptide or a zebrafish Nodal-Related (Squint) polypeptide (e.g., SEQ ID NO:43), or a portion thereof, as defined above. A mutation in a Nodal-Related (squint) nucleic acid molecule can be characterized, for example, by the insertion of a retrovirus into the nucleic acid sequence, for example, using methods described herein. In addition, the invention includes mutations that result in aberrant Nodal-Related (squint) expression or function, including, as examples, null mutations and mutations causing truncations.

By a “Smoothened nucleic acid sequence” is meant a nucleic acid sequence that is, for example, identical to the zebrafish Smoothened nucleic acid sequence of SEQ ID NO:45. Nucleic acid molecules consisting of splice variants of Smoothened nucleic acid sequences, as well as Smoothened nucleic acid sequences containing a mutation, for example, a mutation resulting from the insertion of a virus, e.g., an insertion at nucleotide 271 or 600 of SEQ ID NO:45, are also included in this definition. By a “Smoothened nucleic acid sequence” is also meant a nucleic acid molecule, such as a genomic DNA, cDNA, or RNA (e.g., mRNA) molecule, that encodes a Smoothened polypeptide or a zebrafish Smoothened polypeptide (e.g., SEQ ID NO:46), or a portion thereof, as defined above. A mutation in a Smoothened nucleic acid molecule can be characterized, for example, by the insertion of a retrovirus into the nucleic acid sequence, for example, using methods described herein. In addition, the invention includes mutations that result in aberrant Smoothened expression or function, including, as examples, null mutations and mutations causing truncations.

By a “429 nucleic acid sequence” is meant a nucleic acid sequence that has at least 40%, 50%, 55%, 60%, 70%, 80%, 85%, 90%, 95%, 97%, 99% identity to the zebrafish 429 nucleic acid sequence of SEQ ID NO:47 over at least 50, 100, 200, 500, 1000, 1500, 2000, or 2400 contiguous nucleotides. In a desirable embodiment, a “429 nucleic acid sequence” is identical to the sequence of SEQ ID NO:47. Nucleic acid molecules consisting of splice variants of 429 nucleic acid sequences, as well as 429 nucleic acid sequences containing a mutation, for example, a mutation resulting from the insertion of a virus, e.g., an insertion between nucleotides 182 and 183 of SEQ ID NO:47, are also included in this definition. By a “429 nucleic acid sequence” is also meant a nucleic acid molecule, such as a genomic DNA, cDNA, or RNA (e.g., mRNA) molecule, that encodes a 429 polypeptide or a zebrafish 429 polypeptide (e.g., SEQ ID NO:48), or a portion thereof, as defined above. A mutation in a 429 nucleic acid molecule can be characterized, for example, by the insertion of a retrovirus into the nucleic acid sequence, for example, using methods described herein. In addition, the invention includes mutations that result in aberrant 429 expression or function, including, as examples, null mutations and mutations causing truncations.

By a “428 nucleic acid sequence” is meant a nucleic acid sequence that has at least 85%, 90%, 95%, or 98% identity to the zebrafish 428 nucleic acid sequence of SEQ ID NO:49 over at least 50, 100, 200, 300, 400, 500, 600, or 700 nucleotides. In a desirable embodiment, a “428 nucleic acid sequence” is identical to the sequence of SEQ ID NO:49. Nucleic acid molecules consisting of splice variants of 428 nucleic acid sequences, as well as 428 nucleic acid sequences containing a mutation, for example, a mutation resulting from the insertion of a virus, e.g., an insertion at nucleotide 187 of SEQ ID NO:49, are also included in this definition. By a “428 nucleic acid sequence” is also meant a nucleic acid molecule, such as a genomic DNA, cDNA, or RNA (e.g., mRNA) molecule, that encodes a 428 polypeptide or a zebrafish 428 polypeptide (e.g., SEQ ID NO:50), or a portion thereof, as defined above. A mutation in a 428 nucleic acid molecule can be characterized, for example, by the insertion of a retrovirus into the nucleic acid sequence, for example, using methods described herein. In addition, the invention includes mutations that result in aberrant 428 expression or function, including, as examples, null mutations and mutations causing truncations.

By a “Spinster nucleic acid sequence” is meant a nucleic acid sequence that has at least 83%, 88%, 90%, 95%, or 98% identity to a zebrafish Spinster nucleic acid sequence, for example, that of SEQ ID NO:51. In a desirable embodiment, a “Spinster nucleic acid sequence” is identical to the sequence of SEQ ID NO:51. Nucleic acid molecules consisting of splice variants of Spinster nucleic acid sequences, as well as Spinster nucleic acid sequences containing a mutation, for example, a mutation resulting from the insertion of a virus, e.g., an insertion several kb, for example, 2, 3, 4, 5, 8, 10, or 15 kb upstream of the coding region, are also included in this definition. By a “Spinster nucleic acid sequence” is also meant a nucleic acid molecule, such as a genomic DNA, cDNA, or RNA (e.g., mRNA) molecule, that encodes a Spinster polypeptide or a zebrafish Spinster polypeptide (e.g., SEQ ID NO:52), or a portion thereof, as defined above. A mutation in a Spinster nucleic acid molecule can be characterized, for example, by the insertion of a retrovirus into the nucleic acid sequence, for example, using methods described herein. In addition, the invention includes mutations that result in aberrant Spinster expression or function, including, as examples, null mutations and mutations causing truncations.

By a “Glypican-6 or Knypek nucleic acid sequence” is meant a nucleic acid sequence that has at least 87%, 90%, 95%, or 98% identity to the zebrafish Glypican-6 or Knypek nucleic acid sequence of SEQ ID NO:53 over at least 100, 200, 500, 750, 100, 1500, 1750, or 2000 contiguous nucleic acids. In a desirable embodiment, a “Glypican-6 or Knypek nucleic acid sequence” is identical to the sequence of SEQ ID NO:53. Nucleic acid molecules encoded by splice variants of Glypican-6 or Knypek nucleic acid sequences, as well as Glypican-6 or Knypek nucleic acid sequences containing a mutation, for example, a mutation resulting from the insertion of a virus, e.g., an insertion at nucleotide 133 or 1054 of SEQ ID NO:53, are also included in this definition. By a “Glypican-6 or Knypek nucleic acid sequence” is also meant a nucleic acid molecule, such as a genomic DNA, cDNA, or RNA (e.g., mRNA) molecule, that encodes a Glypican-6 or Knypek polypeptide or a zebrafish Glypican-6 or Knypek polypeptide (e.g., SEQ ID NO:54), or a portion thereof, as defined above. A mutation in a Glypican-6 or knypek nucleic acid molecule can be characterized, for example, by the insertion of a retrovirus into the nucleic acid sequence, for example, using methods described herein. In addition, the invention includes mutations that result in aberrant Glypican-6 or Knypek expression or function, including, as examples, null mutations and mutations causing truncations.

By a “Ribonucleotide Reductase Protein R1 Class 1 nucleic acid sequence” is meant a nucleic acid sequence that is identical to a zebrafish Ribonucleotide Reductase Protein R1 Class 1 nucleic acid sequence, for example, that of SEQ ID NO:55. Nucleic acid molecules consisting of splice variants of Ribonucleotide Reductase Protein R1 Class 1 nucleic acid sequences, as well as Ribonucleotide Reductase Protein R1 Class 1 nucleic acid sequences containing a mutation, for example, a mutation resulting from the insertion of a virus, e.g., an insertion between nucleotides 147 and 148 of SEQ ID NO:55, are also included in this definition. By a “Ribonucleotide Reductase Protein R1 Class 1 nucleic acid sequence” is also meant a nucleic acid molecule, such as a genomic DNA, cDNA, or RNA (e.g., mRNA) molecule, that encodes a Ribonucleotide Reductase Protein R1 Class 1 polypeptide or a zebrafish Ribonucleotide Reductase Protein R1 Class 1 polypeptide (e.g., SEQ ID NO:56), or a portion thereof, as defined above. A mutation in a Ribonucleotide Reductase Protein R1 Class 1 nucleic acid molecule can be characterized, for example, by the insertion of a retrovirus into the nucleic acid sequence, for example, using methods described herein. In addition, the invention includes mutations that result in aberrant Ribonucleotide Reductase Protein R1 Class 1 expression or function, including, as examples, null mutations and mutations causing truncations.

By a “Kinesin Related Motor Protein EG5 nucleic acid sequence” is meant a nucleic acid sequence that has at least 80%, 85%, 90%, 95%, or 98% identity to the zebrafish Kinesin Related Motor Protein EG5 nucleic acid sequence of SEQ ID NO:57 over at least 250, 538, 600, 700, 800, 900, 1000, 1500, 2000, 2500, 3000, or 3500 nucleotides. In a desirable embodiment, a “Kinesin Related Motor Protein EG5 nucleic acid sequence” is identical to the sequence of SEQ ID NO:57. Nucleic acid molecules consisting of splice variants of Kinesin Related Motor Protein EG5 nucleic acid sequences, as well as Kinesin Related Motor Protein EG5 nucleic acid sequences containing a mutation, for example, a mutation resulting from the insertion of a virus, e.g., an insertion between nucleotides 50 and 51 of SEQ ID NO:57, are also included in this definition. By a “Kinesin Related Motor Protein EG5 nucleic acid sequence” is also meant a nucleic acid molecule, such as a genomic DNA, cDNA, or RNA (e.g., mRNA) molecule, that encodes a Kinesin Related Motor Protein EG5 polypeptide or a zebrafish Kinesin Related Motor Protein EG5 polypeptide (e.g., SEQ ID NO:58), or a portion thereof, as defined above. A mutation in a Kinesin Related Motor Protein EG5 nucleic acid molecule can be characterized, for example, by the insertion of a retrovirus into the nucleic acid sequence, for example, using methods described herein. In addition, the invention includes mutations that result in aberrant Kinesin Related Motor Protein EG5 expression or function, including, as examples, null mutations and mutations causing truncations.

By a “Wnt5 (pipetail) nucleic acid sequence” is meant a nucleic acid sequence that is identical to a zebrafish Wnt5 (pipetail) nucleic acid sequence, for example, that of SEQ ID NO:61. Nucleic acid molecules consisting of splice variants of Wnt5 (pipetail) nucleic acid sequences, as well as Wnt5 (pipetail) nucleic acid sequences containing a mutation, for example, a mutation resulting from the insertion of a virus, e.g., an insertion between nucleotides 530 and 531 or at nucleotide 397 of SEQ ID NO:61, are also included in this definition. By a “Wnt5 (pipetail) nucleic acid sequence” is also meant a nucleic acid molecule, such as a genomic DNA, cDNA, or RNA (e.g., mRNA) molecule, that encodes a Wnt5 or Pipetail polypeptide or a zebrafish Wnt5 or Pipetail polypeptide (e.g., SEQ ID NO:62), or a portion thereof, as defined above. A mutation in a Wnt5 (pipetail) nucleic acid molecule can be characterized, for example, by the insertion of a retrovirus into the nucleic acid sequence, for example, using methods described herein. In addition, the invention includes mutations that result in aberrant Wnt5 or Pipetail expression or function, including, as examples, null mutations and mutations causing truncations.

By an “Aryl Hydrocarbon Receptor Nuclear Translocator 2A nucleic acid sequence” is meant a nucleic acid sequence that is identical to a zebrafish Aryl Hydrocarbon Receptor Nuclear Translocator 2A nucleic acid sequence, for example, that of SEQ ID NO:63. Nucleic acid molecules consisting of splice variants of Aryl Hydrocarbon Receptor Nuclear Translocator 2A nucleic acid sequences, as well as Aryl Hydrocarbon Receptor Nuclear Translocator 2A nucleic acid sequences containing a mutation, for example, a mutation resulting from the insertion of a virus, e.g., an insertion at nucleotide 229 or 240 of SEQ ID NO:63, are also included in this definition. By a “Aryl Hydrocarbon Receptor Nuclear Translocator 2A nucleic acid sequence” is also meant a nucleic acid molecule, such as a genomic DNA, cDNA, or RNA (e.g., mRNA) molecule, that encodes a Aryl Hydrocarbon Receptor Nuclear Translocator 2A polypeptide or a zebrafish Aryl Hydrocarbon Receptor Nuclear Translocator 2A polypeptide (e.g., SEQ ID NO:64), or a portion thereof, as defined above. A mutation in a Aryl Hydrocarbon Receptor Nuclear Translocator 2A nucleic acid molecule can be characterized, for example, by the insertion of a retrovirus into the nucleic acid sequence, for example, using methods described herein. In addition, the invention includes mutations that result in aberrant Aryl Hydrocarbon Receptor Nuclear Translocator 2A expression or function, including, as examples, null mutations and mutations causing truncations.

By a “Vesicular Integral-Membrane Protein VIP 36 nucleic acid sequence” is meant a nucleic acid sequence that has at least 76%, 80%, 85%, 90%, 95% or 98% identity to the zebrafish Vesicular Integral-Membrane Protein VIP 36 nucleic acid sequence of SEQ ID NO:65 over at least 100, 200, 271, 300, 400, 500, 600, 700, 800, 900, or 1000 nucleotides. In a desirable embodiment, a “Vesicular Integral-Membrane Protein VIP 36 nucleic acid sequence” is identical to the sequence of SEQ ID NO:65. Nucleic acid molecules consisting of splice variants of Vesicular Integral-Membrane Protein VIP 36 nucleic acid sequences, as well as Vesicular Integral-Membrane Protein VIP 36 nucleic acid sequences containing a mutation, for example, a mutation resulting from the insertion of a virus, e.g., an insertion between nucleotides 219 and 220 of SEQ ID NO:65, are also included in this definition. By a “Vesicular Integral-Membrane Protein VIP 36 nucleic acid sequence” is also meant a nucleic acid molecule, such as a genomic DNA, cDNA, or RNA (e.g., mRNA) molecule, that encodes a Vesicular Integral-Membrane Protein VIP 36 polypeptide or a zebrafish Vesicular Integral-Membrane Protein VIP 36 polypeptide (e.g., SEQ ID NO:66), or a portion thereof, as defined above. A mutation in a Vesicular Integral-Membrane Protein VIP 36 nucleic acid molecule can be characterized, for example, by the insertion of a retrovirus into the nucleic acid sequence, for example, using methods described herein. In addition, the invention includes mutations that result in aberrant Vesicular Integral-Membrane Protein VIP 36 expression or function, including, as examples, null mutations and mutations causing truncations.

By an “299 nucleic acid sequence” is meant a nucleic acid sequence that has at least 84%, 90%, 95% or 98% identity to the zebrafish 299 nucleic acid sequence of SEQ ID NO:67 over at least 89, 250, 500, 750, 1250, 1500, 1750, or 2000 nucleotides. In a desirable embodiment, “299 nucleic acid sequence” is identical to the sequence of SEQ ID NO:67. Nucleic acid molecules consisting of splice variants of 299 nucleic acid sequences, as well as 299 nucleic acid sequences containing a mutation, for example, a mutation resulting from the insertion of a virus, e.g., an insertion between nucleotides 47 and 48 of SEQ ID NO:67, are also included in this definition. By a “299 nucleic acid sequence” is also meant a nucleic acid molecule, such as a genomic DNA, cDNA, or RNA (e.g., mRNA) molecule, that encodes a 299 polypeptide or a zebrafish 299 polypeptide (e.g., SEQ ID NO:68), or a portion thereof, as defined above. A mutation in a 299 nucleic acid molecule can be characterized, for example, by the insertion of a retrovirus into the nucleic acid sequence, for example, using methods described herein. In addition, the invention includes mutations that result in aberrant 299 expression or function, including, as examples, null mutations and mutations causing truncations.

By a “994 nucleic acid sequence” is meant a nucleic acid sequence that has at least 40%, 45%, 50%, 60%, 70%, 80%, 90%, 95%, or 98% identity to the zebrafish 994 nucleic acid sequence of SEQ ID NO:69 over at least 100, 150, 200, 250, 400, 500, 750, 1000, 1250, or 1500 contiguous nucleotides. In a desirable embodiment, a “994 nucleic acid sequence” is identical to the sequence of SEQ ID NO:69. Nucleic acid molecules consisting of splice variants of 994 nucleic acid sequences, as well as 994 nucleic acid sequences containing a mutation, for example, a mutation resulting from the insertion of a virus, e.g., an insertion between nucleotides 66 and 67 of SEQ ID NO:69, are also included in this definition. By a “994 nucleic acid sequence” is also meant a nucleic acid molecule, such as a genomic DNA, cDNA, or RNA (e.g., mRNA) molecule, that encodes a 994 polypeptide or a zebrafish 994 polypeptide (e.g., SEQ ID NO:70), or a portion thereof, as defined above. A mutation in a 994 nucleic acid molecule can be characterized, for example, by the insertion of a retrovirus into the nucleic acid sequence, for example, using methods described herein. In addition, the invention includes mutations that result in aberrant 994 expression or function, including, as examples, null mutations and mutations causing truncations.

By a “1373 nucleic acid sequence” is meant a nucleic acid sequence that has at least 84%, 90%, 95% or 98% identity to the zebrafish 1373 nucleic acid sequence of SEQ ID NO:71 over at least 333, 400, 450, or 500 nucleotides. In a desirable embodiment, a a “1373 nucleic acid sequence” is identical to the sequence of SEQ ID NO:71. Nucleic acid molecules consisting of splice variants of 1373 nucleic acid sequences, as well as 1373 nucleic acid sequences containing a mutation, for example, a mutation resulting from the insertion of a virus, e.g., an insertion between nucleotides 118 and 119 of SEQ ID NO:71, are also included in this definition. By a “1373 nucleic acid sequence” is also meant a nucleic acid molecule, such as a genomic DNA, cDNA, or RNA (e.g., mRNA) molecule, that encodes a 1373 polypeptide or a zebrafish 1373 polypeptide (e.g., SEQ ID NO:72), or a portion thereof, as defined above. A mutation in a 1373 nucleic acid molecule can be characterized, for example, by the insertion of a retrovirus into the nucleic acid sequence, for example, using methods described herein. In addition, the invention includes mutations that result in aberrant 1373 expression or function, including, as examples, null mutations and mutations causing truncations.

By a “Denticleless nucleic acid sequence” is meant a nucleic acid sequence that has at least 79%, 85%, 90%, 95%, or 98% identity to the zebrafish Denticleless nucleic acid sequence of SEQ ID NO:73 over at least 181 nucleotides. In a desirable embodiment, a “Denticleless nucleic acid sequence” is identical to the sequence of SEQ ID NO:73. Nucleic acid molecules consisting of splice variants of Denticleless nucleic acid sequences, as well as Denticleless nucleic acid sequences containing a mutation, for example, a mutation resulting from the insertion of a virus, e.g., an insertion between nucleotides 307 and 308 of SEQ ID NO:73, are also included in this definition. By a “Denticleless nucleic acid sequence” is also meant a nucleic acid molecule, such as a genomic DNA, cDNA, or RNA (e.g., mRNA) molecule, that encodes a Denticleless polypeptide or a zebrafish Denticleless polypeptide (e.g., SEQ ID NO:74), or a portion thereof, as defined above. A mutation in a Denticleless nucleic acid molecule can be characterized, for example, by the insertion of a retrovirus into the nucleic acid sequence, for example, using methods described herein. In addition, the invention includes mutations that result in aberrant Denticleless expression or function, including, as examples, null mutations and mutations causing truncations.

By a “Ribonucleotide Reductase Protein R2 nucleic acid sequence” is meant a nucleic acid sequence that is identical to the zebrafish Ribonucleotide Reductase Protein R2 nucleic acid sequence, for example, that of SEQ ID NO:75. In a desirable embodiment, a “Ribonucleotide Reductase Protein R2 nucleic acid sequence” is identical to the sequence of SEQ ID NO:75. Nucleic acid molecules consisting of splice variants of Ribonucleotide Reductase Protein R2 nucleic acid sequences, as well as Ribonucleotide Reductase Protein R2 nucleic acid sequences containing a mutation, for example, a mutation resulting from the insertion of a virus, e.g., an insertion at nucleotide 137 of SEQ ID NO:75 (which corresponds to position 360 of GenBank Accession No. AW280665), or at 337 or 342 of AW28066 are also included in this definition. By a “Ribonucleotide Reductase Protein R2 nucleic acid sequence” is also meant a nucleic acid molecule, such as a genomic DNA, cDNA, or RNA (e.g., mRNA) molecule, that encodes a Ribonucleotide Reductase Protein R2 polypeptide or a zebrafish a Ribonucleotide Reductase Protein R2 polypeptide (e.g., SEQ ID NO:76), or a portion thereof, as defined above. A mutation in a Ribonucleotide Reductase Protein R2 nucleic acid molecule can be characterized, for example, by the insertion of a retrovirus into the nucleic acid sequence, for example, using methods described herein. In addition, the invention includes mutations that result in aberrant Ribonucleotide Reductase Protein R2 expression or function, including, as examples, null mutations and mutations causing truncations.

By a “TCP-1 Alpha nucleic acid sequence” is meant a nucleic acid sequence that is identical to a zebrafish TCP-1 Alpha nucleic acid sequence, for example, that of SEQ ID NO:77. Nucleic acid molecules consisting of splice variants of TCP-1 Alpha nucleic acid sequences, as well as TCP-1 Alpha nucleic acid sequences containing a mutation, for example, a mutation resulting from the insertion of a virus, e.g., an insertion 140 bp upstream of the coding region of between nucleotides 130 and 131 of SEQ ID NO:77, are also included in this definition. By a “TCP-1 Alpha nucleic acid sequence” is also meant a nucleic acid molecule, such as a genomic DNA, cDNA, or RNA (e.g., mRNA) molecule, that encodes a TCP-1 Alpha polypeptide or a zebrafish TCP-1 Alpha polypeptide (e.g., SEQ ID NO:78), or a portion thereof, as defined above. A mutation in a TCP-1 Alpha nucleic acid molecule can be characterized, for example, by the insertion of a retrovirus into the nucleic acid sequence, for example, using methods described herein. In addition, the invention includes mutations that result in aberrant TCP-1 Alpha expression or function, including, as examples, null mutations and mutations causing truncations.

By a “Telomeric Repeat Factor 2 nucleic acid sequence” is meant a nucleic acid sequence that has at least 31%, 40%, 50%, 60%, 75%, 85%, 90%, 95% or 98% identity to the zebrafish Telomeric Repeat Factor 2 nucleic acid sequence of SEQ ID NO:79 over at least 354, 500, 1000, 1500, 1700, 2000, or 2200 nucleotides. In a desirable embodiment, a “Telomeric Repeat Factor 2 nucleic acid sequence” is identical to the sequence of SEQ ID NO:79. Nucleic acid molecules consisting of splice variants of Telomeric Repeat Factor 2 nucleic acid sequences, as well as telomeric repeatfactor 2 nucleic acid sequences containing a mutation, for example, a mutation resulting from the insertion of a virus, e.g., an insertion between nucleotides 529 and 530 of SEQ ID NO:79, are also included in this definition. By a “Telomeric Repeat Factor 2 nucleic acid sequence” is also meant a nucleic acid molecule, such as a genomic DNA, cDNA, or RNA (e.g., mRNA) molecule, that encodes a Telomeric Repeat Factor 2 polypeptide or a zebrafish Telomeric Repeat Factor 2 polypeptide (e.g., SEQ ID NO:80), or a portion thereof, as defined above. A mutation in a Telomeric Repeat Factor 2 nucleic acid molecule can be characterized, for example, by the insertion of a retrovirus into the nucleic acid sequence, for example, using methods described herein. In addition, the invention includes mutations that result in aberrant Telomeric Repeat Factor 2 expression or function, including, as examples, null mutations and mutations causing truncations.

By a “SIL nucleic acid sequence” is meant a nucleic acid sequence that has at least 79%, 85%, 90%, 95%, or 98% identity to the zebrafish SIL nucleic acid sequence of SEQ ID NO:81 over at least 96, 200, 500, 1000, 1500, 2000, 2500, 3000, 3500, or 4000 nucleotides. In a desirable embodiment, a “SIL nucleic acid sequence” is identical to the sequence of SEQ ID NO:81. Nucleic acid molecules consisting of splice variants of SIL nucleic acid sequences, as well as SIL nucleic acid sequences containing a mutation, for example, a mutation resulting from the insertion of a virus, e.g., an insertion between nucleotides 273 and 274 of SEQ ID NO:81, are also included in this definition. By a “SIL nucleic acid sequence” is also meant a nucleic acid molecule, such as a genomic DNA, cDNA, or RNA (e.g., mRNA) molecule, that encodes a SIL polypeptide or a zebrafish SIL polypeptide (e.g., SEQ ID NO:82), or a portion thereof, as defined above. A mutation in a SIL nucleic acid molecule can be characterized, for example, by the insertion of a retrovirus into the nucleic acid sequence, for example, using methods described herein. In addition, the invention includes mutations that result in aberrant SIL expression or function, including, as examples, null mutations and mutations causing truncations.

By a “U1 Small Nuclear Ribonucleoprotein C nucleic acid sequence” is meant a nucleic acid sequence that has at least 85%, 90%, 95% or 98% identity to the zebrafish U1 Small Nuclear Ribonucleoprotein C nucleic acid sequence of SEQ ID NO:83 over at least 170, 250, 300, 400, 500, 600, 700, or 750 nucleotides. In a desirable embodiment, a “U1 Small Nuclear Ribonucleoprotein C nucleic acid sequence” is identical to the sequence of SEQ ID NO:83. Nucleic acid molecules consisting of splice variants of U1 Small Nuclear Ribonucleoprotein C nucleic acid sequences, as well as U1 Small Nuclear Ribonucleoprotein C nucleic acid sequences containing a mutation, for example, a mutation resulting from the insertion of a virus, e.g., an insertion between nucleotides 52 and 53 of SEQ ID NO:83, are also included in this definition. By a “U1 Small Nuclear Ribonucleoprotein C nucleic acid sequence” is also meant a nucleic acid molecule, such as a genomic DNA, cDNA, or RNA (e.g., mRNA) molecule, that encodes a U1 Small Nuclear Ribonucleoprotein C polypeptide or a zebrafish (e.g., SEQ ID NO:84) U1 Small Nuclear Ribonucleoprotein C polypeptide, or a portion thereof, as defined above. A mutation in a U1 Small Nuclear Ribonucleoprotein C nucleic acid molecule can be characterized, for example, by the insertion of a retrovirus into the nucleic acid sequence, for example, using methods described herein. In addition, the invention includes mutations that result in aberrant U1 Small Nuclear Ribonucleoprotein C expression or function, including, as examples, null mutations and mutations causing truncations.

By a “Ski Interacting Protein (SKIP) nucleic acid sequence” is meant a nucleic acid sequence that has at least 79%, 85%, 90%, 95% or 98% identity to the zebrafish Ski Interacting Protein (SKIP) nucleic acid sequence of SEQ ID NO:85 over at least 500, 600, 700, 812, 900, 1000, 1500, or 2000 nucleotides. In a desirable embodiment, a “Ski Interacting Protein (SKIP) nucleic acid sequence” is identical to the sequence of SEQ ID NO:85. Nucleic acid molecules consisting of splice variants of Ski Interacting Protein (SKIP) nucleic acid sequences, as well as Ski Interacting Protein (SKIP) nucleic acid sequences containing a mutation, for example, a mutation resulting from the insertion of a virus, e.g., an insertion 1.2 kb upstream of the coding region of the Ski Interacting Protein (SKIP) gene, are also included in this definition. By a “Ski Interacting Protein (SKIP) nucleic acid sequence” is also meant a nucleic acid molecule, such as a genomic DNA, cDNA, or RNA (e.g., mRNA) molecule, that encodes a Ski Interacting Protein (SKIP) polypeptide or a zebrafish Ski Interacting Protein (SKIP) (e.g., SEQ ID NO:86), or a portion thereof, as defined above. A mutation in a Ski Interacting Protein (SKIP) nucleic acid molecule can be characterized, for example, by the insertion of a retrovirus into the nucleic acid sequence, for example, using methods described herein. In addition, the invention includes mutations that result in aberrant Ski Interacting Protein (SKIP) expression or function, including, as examples, null mutations and mutations causing truncations.

By a “297 nucleic acid sequence” is meant a nucleic acid sequence that has at least 80%, 85%, 90%, 95% or 98% identity to the zebrafish 297 nucleic acid sequence of SEQ ID NO:87 over at least 173, 250, 300, 400, 500, or 600 nucleotides. In a desirable embodiment, a “297 nucleic acid sequence” is identical to the sequence of SED ID NO:87. Nucleic acid molecules encoded by splice variants of 297 nucleic acid sequences, as well as 297 nucleic acid sequences containing a mutation, for example, a mutation resulting from the insertion of a virus, e.g., an insertion at nucleotide 74 of SEQ ID NO:87, are also included in this definition. By a “297 nucleic acid sequence” is also meant a nucleic acid molecule, such as a genomic DNA, cDNA, or RNA (e.g., mRNA) molecule, that encodes a 297 polypeptide or a zebrafish 297 polypeptide (e.g., SEQ ID NO:88), or a portion thereof, as defined above. A mutation in a 297 nucleic acid molecule can be characterized, for example, by the insertion of a retrovirus into the nucleic acid sequence, for example, using methods described herein. In addition, the invention includes mutations that result in aberrant 297 expression or function, including, as examples, null mutations and mutations causing truncations.

By a “TCP-1 Complex Gamma Chain nucleic acid sequence” is meant a nucleic acid sequence that has at least 76%, 80%, 85%, 90% or 95% identity to the zebrafish TCP-1 Complex Gamma Chain nucleic acid sequence of SEQ ID NO:89 over at least 250, 300, 400, 500, 600, 700, 800, 900, 1000, or 1598 nucleotides. In a desirable embodiment, a “TCP-1 Complex Gamma Chain nucleic acid sequence” is identical to the sequence of SEQ ID NO:89. Nucleic acid molecules consisting of splice variants of TCP-1 Complex Gamma Chain nucleic acid sequences, as well as TCP-1 Complex Gamma Chain nucleic acid sequences containing a mutation, for example, a mutation resulting from the insertion of a virus, e.g., an insertion between nucleotide 75 and 76 of SEQ ID NO:89, are also included in this definition. By a “TCP-1 Complex Gamma Chain nucleic acid sequence” is also meant a nucleic acid molecule, such as a genomic DNA, cDNA, or RNA (e.g., mRNA) molecule, that encodes a TCP-1 Complex Gamma Chain polypeptide or a zebrafish TCP-1 Complex Gamma Chain polypeptide (e.g., SEQ ID NO:90), or a portion thereof, as defined above. A mutation in a TCP-1 Complex Gamma Chain nucleic acid molecule can be characterized, for example, by the insertion of a retrovirus into the nucleic acid sequence, for example, using methods described herein. In addition, the invention includes mutations that result in aberrant TCP-1 Complex Gamma Chain expression or function, including, as examples, null mutations and mutations causing truncations.

By a “Small Nuclear Ribonucleoprotein D1 nucleic acid sequence” is meant a nucleic acid sequence that has at least 88%, 90%, 95%, or 98% identity to the zebrafish Small Nuclear Ribonucleoprotein D1 nucleic acid sequence of SEQ ID NO:91 over at least 152, 250, 300, 400, 500, 600, 700, 800, 900, 1000, or 1500 nucleotides. In a desirable embodiment, a “Small Nuclear Ribonucleoprotein D1 nucleic acid sequence” is identical to the sequence of SEQ ID NO:91. Nucleic acid molecules consisting of splice variants of Small Nuclear Ribonucleoprotein D1 nucleic acid sequences, as well as Small Nuclear Ribonucleoprotein D1 nucleic acid sequences containing a mutation, for example, a mutation resulting from the insertion of a virus, e.g., an insertion between nucleotides 76 and 77 of SEQ ID NO:91, are also included in this definition. By a “Small Nuclear Ribonucleoprotein D1 nucleic acid sequence” is also meant a nucleic acid molecule, such as a genomic DNA, cDNA, or RNA (e.g., mRNA) molecule, that encodes a Small Nuclear Ribonucleoprotein D1polypeptide or a zebrafish Small Nuclear Ribonucleoprotein D1 polypeptide (e.g., SEQ ID NO:92), or a portion thereof, as defined above. A mutation in a Small Nuclear Ribonucleoprotein D1 nucleic acid molecule can be characterized, for example, by the insertion of a retrovirus into the nucleic acid sequence, for example, using methods described herein. In addition, the invention includes mutations that result in aberrant Small Nuclear Ribonucleoprotein D1 expression or function, including, as examples, null mutations and mutations causing truncations.

By a “DNA Polymerase Epsilon Subunit B nucleic acid sequence” is meant a nucleic acid sequence that has at least 70%, 75%, 80%, 85%, 90%, 95%, or 98% identity to the zebrafish DNA Polymerase Epsilon Subunit B nucleic acid sequence of SEQ ID NO:93 over at least 500, 1038, 1500, or 2000 nucleotides, or at least 79%, 85%, 90%, 95%, or 99% identity or over at least 96, 250, 300, 400, 500, 600, 700, 800, 900, 1000, 1500, or 1750 nucleotides. In a desirable embodiment, a “DNA Polymerase Epsilon Subunit B nucleic acid sequence” is identical to the sequence of SEQ ID NO:93. Nucleic acid molecules consisting of splice variants of DNA Polymerase Epsilon Subunit B nucleic acid sequences, as well as DNA Polymerase Epsilon Subunit B nucleic acid sequences containing a mutation, for example, a mutation resulting from the insertion of a virus, e.g., an insertion between nucleotide 1161 and 1162, or at nucleotide 929 of SEQ ID NO:93, are also included in this definition. By a “DNA Polymerase Epsilon Subunit B nucleic acid sequence” is also meant a nucleic acid molecule, such as a genomic DNA, cDNA, or RNA (e.g., mRNA) molecule, that encodes a DNA Polymerase Epsilon Subunit B polypeptide or a zebrafish DNA Polymerase Epsilon Subunit B (e.g., SEQ ID NO:94), or a portion thereof, as defined above. A mutation in a DNA Polymerase Epsilon Subunit B nucleic acid molecule can be characterized, for example, by the insertion of a retrovirus into the nucleic acid sequence, for example, using methods described herein. In addition, the invention includes mutations that result in aberrant DNA Polymerase Epsilon Subunit B expression or function, including, as examples, null mutations and mutations causing truncations.

By a “821-02 nucleic acid sequence” is meant a nucleic acid sequence that has at least 76%, 80%, 85%, 90%, 95%, or 98% identity to the zebrafish 821-02 nucleic acid sequence of SEQ ID NO:95 over at least 99, 250, 300, 400, 500, 600, 700, 800, 900, 1000, 1500, 2000, or 2500 nucleotides. In a desirable embodiment, a “821-02 nucleic acid sequence” is identical to the sequence of SEQ ID NO:95. Nucleic acid molecules consisting of splice variants of 821-02 nucleic acid sequences, as well as 821-02 nucleic acid sequences containing a mutation, for example, a mutation resulting from the insertion of a virus, e.g., an insertion between nucleotides 369 and 370 or 231 and 232 of SEQ ID NO:95, are also included in this definition. By a “821-02 nucleic acid molecule” is also meant a nucleic acid molecule, such as a genomic DNA, cDNA, or RNA (e.g., mRNA) molecule, that encodes a 821-02 polypeptide or a zebrafish 821-02 polypeptide (e.g., SEQ ID NO:96), or a portion thereof, as defined above. A mutation in a 821-02 nucleic acid molecule can be characterized, for example, by the insertion of a retrovirus into the nucleic acid sequence, for example, using methods described herein. In addition, the invention includes mutations that result in aberrant 821-02 expression or function, including, as examples, null mutations and mutations causing truncations.

By a “1045 nucleic acid sequence” is meant a nucleic acid sequence that has at least 75%, 80%, 85%, 90%, or 95% identity to the zebrafish 1045 nucleic acid sequence of SEQ ID NO:97 over at least 250, 573, 700, 800, 900, or 1000 nucleotides. In a desirable embodiment, a “1045 nucleic acid sequence” is identical to the sequence of SEQ ID NO:97. Nucleic acid molecules consisting of splice variants of 1045 nucleic acid sequences, as well as 1045 nucleic acid sequences containing a mutation, for example, a mutation resulting from the insertion of a virus, e.g., an insertion between nucleotides 216 and 344 of SEQ ID NO:97, are also included in this definition. By a “1045 nucleic acid sequence” is also meant a nucleic acid molecule, such as a genomic DNA, cDNA, or RNA (e.g., mRNA) molecule, that encodes a 1045 polypeptide or a zebrafish 1045 polypeptide (e.g., SEQ ID NO:98), or a portion thereof, as defined above. A mutation in a 1045 nucleic acid molecule can be characterized, for example, by the insertion of a retrovirus into the nucleic acid sequence, for example, using methods described herein. In addition, the invention includes mutations that result in aberrant 1045 expression or function, including, as examples, null mutations and mutations causing truncations.

By a “1055-1 nucleic acid sequence” is meant a nucleic acid sequence that has at least 74%, 80%, 85%, 90%, or 95% identity to the zebrafish 1055-1 nucleic acid sequence of SEQ ID NO:99 over at least 250, 552, 600, 700, 800, 900, 1000, 1500, or 2000 nucleotides. In a desirable embodiment, a “1055-1 nucleic acid sequence” is identical to the sequence of SEQ ID NO:99. Nucleic acid molecules consisting of splice variants of 1055-1 nucleic acid sequences, as well as 1055-1 nucleic acid sequences containing a mutation, for example, a mutation resulting from the insertion of a virus, e.g., an insertion between nucleotides 167 and 168 of SEQ ID NO:99, are also included in this definition. By a “1055-1 nucleic acid sequence” is also meant a nucleic acid molecule, such as a genomic DNA, cDNA, or RNA (e.g., mRNA) molecule, that encodes a 1055-1 polypeptide or a zebrafish 1055-1 polypeptide (e.g., SEQ ID NO:100), or a portion thereof, as defined above. A mutation in a 1055-1 nucleic acid molecule can be characterized, for example, by the insertion of a retrovirus into the nucleic acid sequence, for example, using methods described herein. In addition, the invention includes mutations that result in aberrant 1055-1 expression or function, including, as examples, null mutations and mutations causing truncations.

By a “Spliceosome Associated Protein 49 nucleic acid sequence” is meant a nucleic acid sequence that has at least 78%, 80%, 85%, 90%, or 95% identity to the zebrafish Spliceosome Associated Protein 49 nucleic acid sequence of SEQ ID NO:101 over at least 250, 500, 651, 700, 800, 900, 1000, or 1200 nucleotides. In a desirable embodiment, a “Spliceosome Associated Protein 49 nucleic acid sequence” is identical to the sequence of SEQ ID NO:101. Nucleic acid molecules consisting of splice variants of Spliceosome Associated Protein 49 nucleic acid sequences, as well as Spliceosome Associated Protein 49 nucleic acid sequences containing a mutation, for example, a mutation resulting from the insertion of a virus, e.g., an insertion between nucleotides 53 and 54 of SEQ ID NO:101, are also included in this definition. By a “Spliceosome Associated Protein 49 nucleic acid sequence” is also meant a nucleic acid molecule, such as a genomic DNA, cDNA, or RNA (e.g., mRNA) molecule, that encodes a Spliceosome Associated Protein 49 polypeptide or a zebrafish Spliceosome Associated Protein 49 polypeptide (e.g., SEQ ID NO:102), or a portion thereof, as defined above. A mutation in a Spliceosome Associated Protein 49 nucleic acid molecule can be characterized, for example, by the insertion of a retrovirus into the nucleic acid sequence, for example, using methods described herein. In addition, the invention includes mutations that result in aberrant Spliceosome Associated Protein 49 expression or function, including, as examples, null mutations and mutations causing truncations.

By a “DNA Replication Licensing Factor MCM7 nucleic acid sequence” is meant a nucleic acid sequence that has at least 74%, 80%, 85%, 90%, 95%, or 98% identity to the zebrafish DNA Replication Licensing Factor MCM7 nucleic acid sequence of SEQ ID NO:103 over at least 100, 200, 286, 400, 500, 600, 700, or 800 nucleotides. In a desirable embodiment, a “DNA Replication Licensing Factor MCM7 nucleic acid sequence” is identical to the sequence of SEQ ID NO:103. Nucleic acid molecules consisting of splice variants of DNA Replication Licensing Factor MCM7 nucleic acid sequences, as well as DNA Replication Licensing Factor MCM7 nucleic acid sequences containing a mutation, for example, a mutation resulting from the insertion of a virus, e.g., an insertion between nucleotides 121 and 122 or at nucleotide 198 of SEQ ID NO:103, are also included in this definition. By a “DNA Replication Licensing Factor MCM7 nucleic acid sequence” is also meant a nucleic acid molecule, such as a genomic DNA, cDNA, or RNA (e.g., mRNA) molecule, that encodes a DNA Replication Licensing Factor MCM7 polypeptide or a zebrafish DNA Replication Licensing Factor MCM7 polypeptide (e.g., SEQ ID NO:104), or a portion thereof, as defined above. A mutation in a DNA Replication Licensing Factor MCM7 nucleic acid molecule can be characterized, for example, by the insertion of a retrovirus into the nucleic acid sequence, for example, using methods described herein. In addition, the invention includes mutations that result in aberrant DNA Replication Licensing Factor MCM7 expression or function, including, as examples, null mutations and mutations causing truncations.

By a “Dead-Box RNA Helicase (DEAD5 or DEAD19) nucleic acid sequence” is meant a nucleic acid sequence that has at least 81%, 85%, 90%, 95%, or 98% identity to the zebrafish DNA Dead-Box RNA Helicase (DEAD5 or DEAD19) nucleic acid sequence of SEQ ID NO:105 over at least 250, 300, 400, 500, 600, 700, 800, 810, 900, 1000, 1500, or 1750 nucleotides. In a desirable embodiment, a “Dead-Box RNA Helicase (DEAD5 or DEAD19) nucleic acid sequence” is identical to the sequence of SEQ ID NO:105. Nucleic acid molecules encoded by splice variants of Dead-Box RNA Helicase (DEAD5 or DEAD19) nucleic acid sequences, as well as Dead-Box RNA Helicase (DEAD5 or DEAD19) nucleic acid sequences containing a mutation, for example, a mutation resulting from the insertion of a virus, e.g., an insertion at nucleotide 132 of SEQ ID NO:105, are also included in this definition. By a “Dead-Box RNA Helicase (DEAD5 or DEAD19) nucleic acid sequence” is also meant a nucleic acid molecule, such as a genomic DNA, cDNA, or RNA (e.g., mRNA) molecule, that encodes a Dead-Box RNA Helicase (DEAD5 or DEAD19) polypeptide or a zebrafish Dead-Box RNA Helicase (DEAD5 or DEAD19) polypeptide (e.g., SEQ ID NO:106), or a portion thereof, as defined above. A mutation in a Dead-Box RNA Helicase (DEAD5 or DEAD19) nucleic acid molecule can be characterized, for example, by the insertion of a retrovirus into the nucleic acid sequence, for example, using methods described herein. In addition, the invention includes mutations that result in aberrant Dead-Box RNA Helicase (DEAD5 or DEAD19) expression or function, including, as examples, null mutations and mutations causing truncations.

By a “1581 nucleic acid sequence” is meant a nucleic acid sequence that has at least 77%, 80%, 85%, 90%, 95%, or 98% identity to the zebrafish 1581 nucleic acid sequence of SEQ ID NO:107 over at least 165, 250, 300, 400, 500, 600, 700, 800, 900, 1000, or 1300 nucleotides. In a desirable embodiment, a “1581 nucleic acid sequence” is identical to the sequence of SEQ ID NO:107. Nucleic acid molecules consisting of splice variants of 1581 nucleic acid sequences, as well as 1581 nucleic acid sequences containing a mutation, for example, a mutation resulting from the insertion of a virus, e.g., an insertion between nucleotides 346 and 347 of SEQ ID NO:107, are also included in this definition. By a “1581 nucleic acid sequence” is also meant a nucleic acid molecule, such as a genomic DNA, cDNA, or RNA (e.g., mRNA) molecule, that encodes a 1581 polypeptide or a zebrafish 1581 polypeptide (e.g., SEQ ID NO:108), or a portion thereof, as defined above. A mutation in a 1581 nucleic acid molecule can be characterized, for example, by the insertion of a retrovirus into the nucleic acid sequence, for example, using methods described herein. In addition, the invention includes mutations that result in aberrant 1581 expression or function, including, as examples, null mutations and mutations causing truncations.

By a “Cyclin A2 nucleic acid sequence” is meant a nucleic acid sequence that is, for example, identical to the zebrafish Cyclin A2 nucleic acid sequence of SEQ ID NO: 109. Nucleic acid molecules consisting of splice variants of Cyclin A2 nucleic acid sequences, as well as Cyclin A2 nucleic acid sequences containing a mutation, for example, a mutation resulting from the insertion of a virus, e.g., an insertion at nucleotide 374 or 401 of SEQ ID NO:109, are also included in this definition. By a “Cyclin A2 nucleic acid sequence” is also meant a nucleic acid molecule, such as a genomic DNA, cDNA, or RNA (e.g., mRNA) molecule, that encodes a Cyclin A2 polypeptide or a zebrafish (e.g., SEQ ID NO:110) Cyclin A2 polypeptide, or a portion thereof, as defined above. A mutation in a Cyclin A2 nucleic acid molecule can be characterized, for example, by the insertion of a retrovirus into the nucleic acid sequence, for example, using methods described herein. In addition, the invention includes mutations that result in aberrant Cyclin A2 expression or function, including, as examples, null mutations and mutations causing truncations.

By an “Imitation Switch (ISWI)/SNF2 nucleic acid sequence” or “ISWI/SNF2 nucleic acid sequence” is meant a nucleic acid sequence that has at least 84%, 90%, 95%, or 98% identity to the zebrafish Imitation Switch (ISWI)/SNF2 nucleic acid sequence of SEQ ID NO:11 over at least 196, 250, 300, 400, 500, 600, 700, 800, 900, 1000, or 1100 nucleotides. In a desirable embodiment, an “Imitation Switch (ISWI)/SNF2 nucleic acid sequence” is identical to the sequence of SEQ ID NO:111. Nucleic acid molecules consisting of splice variants of Imitation Switch (ISWI)/SNF2 nucleic acid sequences, as well as Imitation Switch (ISWI)/SNF2 nucleic acid sequences containing a mutation, for example, a mutation resulting from the insertion of a virus, e.g., an insertion at nucleotide 76 of SEQ ID NO:111, are also included in this definition. By an “Imitation Switch (ISWI)/SNF2 nucleic acid sequence” is also meant a nucleic acid molecule, such as a genomic DNA, cDNA, or RNA (e.g., mRNA) molecule, that encodes an Imitation Switch (ISWI)/SNF2 polypeptide or a zebrafish Imitation Switch (ISWI)/SNF2 polypeptide (e.g., SEQ ID NO:112), or a portion thereof, as defined above. A mutation in an Imitation Switch (ISWI)/SNF2 nucleic acid molecule can be characterized, for example, by the insertion of a retrovirus into the nucleic acid sequence, for example, using methods described herein. In addition, the invention includes mutations that result in aberrant Imitation Switch (ISWI)/SNF2 expression or function, including, as examples, null mutations and mutations causing truncations.

By a “Chromosomal Assembly Protein C (XCAP-C) nucleic acid sequence” or “XCAP-C nucleic acid sequence” is meant a nucleic acid sequence that has at least 78%, 85%, 90%, 95%, or 98% identity to the zebrafish Chromosomal Assembly Protein C (XCAP-C) nucleic acid sequence of SEQ ID NO:113 over at least 250, 500, 765, 800, 900, 1000, 1250, 1500, 2000, 2500, 3000, 3500, or 4000 nucleotides. In a desirable embodiment, a “Chromosomal Assembly Protein C (XCAP-C) nucleic acid sequence” is identical to the sequence of SEQ ID NO:113. Nucleic acid molecules consisting of splice variants of Chromosomal Assembly Protein C (XCAP-C) nucleic acid sequences, as well as Chromosomal Assembly Protein C (XCAP-C) nucleic acid sequences containing a mutation, for example, a mutation resulting from the insertion of a virus, e.g., an insertion between nucleotides 181 and 182 of SEQ ID NO:113, are also included in this definition. By a “Chromosomal Assembly Protein C (XCAP-C) nucleic acid sequence” is also meant a nucleic acid molecule, such as a genomic DNA, cDNA, or RNA (e.g., mRNA) molecule, that encodes Chromosomal Assembly Protein C (XCAP-C) polypeptide or a zebrafish (e.g., SEQ ID NO:114) Chromosomal Assembly Protein C (XCAP-C) polypeptide, or a portion thereof, as defined above. A mutation in a Chromosomal Assembly Protein C (XCAP-C) nucleic acid molecule can be characterized, for example, by the insertion of a retrovirus into the nucleic acid sequence, for example, using methods described herein. In addition, the invention includes mutations that result in aberrant Chromosomal Assembly Protein C (XCAP-C) expression or function, including, as examples, null mutations and mutations causing truncations.

By a “DNA Replication Licensing Factor MCM2 nucleic acid sequence” is meant a nucleic acid sequence that has at least 78%, 85%, 90%, 95%, or 98% identity to the zebrafish DNA Replication Licensing Factor MCM2 nucleic acid sequence of SEQ ID NO:115 over at least 500, 1000, 1164, 1500, or 2000 nucleotides. In a desirable embodiment, a “DNA Replication Licensing Factor MCM2 nucleic acid sequence” is identical to the sequence of SEQ ID NO:115. Nucleic acid molecules consisting of splice variants of DNA Replication Licensing Factor MCM2 nucleic acid sequences, as well as DNA Replication Licensing Factor MCM2 nucleic acid sequences containing a mutation, for example, a mutation resulting from the insertion of a virus, e.g., an insertion in the intron preceding nucleotide 399 of SEQ ID NO:115, are also included in this definition. By a “DNA Replication Licensing Factor MCM2 nucleic acid sequence” is also meant a nucleic acid molecule, such as a genomic DNA, cDNA, or RNA (e.g., mRNA) molecule, that encodes DNA Replication Licensing Factor MCM2 polypeptide or a zebrafish DNA Replication Licensing Factor MCM2 polypeptide (e.g., SEQ ID NO:116), or a portion thereof, as defined above. A mutation in a DNA Replication Licensing Factor MCM2 nucleic acid molecule can be characterized, for example, by the insertion of a retrovirus into the nucleic acid sequence, for example, using methods described herein. In addition, the invention includes mutations that result in aberrant DNA Replication Licensing Factor MCM2 expression or function, including, as examples, null mutations and mutations causing truncations.

By a “DNA Replication Licensing Factor MCM3 nucleic acid sequence” is meant a nucleic acid sequence that has at least 78%, 85%, 90%, or 95% identity to the zebrafish DNA Replication Licensing Factor MCM3 nucleic acid sequence of SEQ ID NO:117 over at least 250, 400, 500, 574, or 600 nucleotides. In a desirable embodiment, a “DNA Replication Licensing Factor MCM3 nucleic acid sequence” is identical to the sequence of SEQ ID NO:117. Nucleic acid molecules consisting of splice variants of DNA Replication Licensing Factor MCM3 nucleic acid sequences, as well as DNA Replication Licensing Factor MCM3 nucleic acid sequences containing a mutation, for example, a mutation resulting from the insertion of a virus, e.g., an insertion at nucleotide 50 or between nucleotides 75 and 76 of SEQ ID NO:117, are also included in this definition. By a “DNA Replication Licensing Factor MCM3 nucleic acid sequence” is also meant a nucleic acid molecule, such as a genomic DNA, cDNA, or RNA (e.g., mRNA) molecule, that encodes DNA Replication Licensing Factor MCM3 polypeptide or a zebrafish DNA Replication Licensing Factor MCM3 polypeptide (e.g., SEQ ID NO:118), or a portion thereof, as defined above. A mutation in a DNA Replication Licensing Factor MCM3 nucleic acid molecule can be characterized, for example, by the insertion of a retrovirus into the nucleic acid sequence, for example, using methods described herein. In addition, the invention includes mutations that result in aberrant DNA Replication Licensing Factor MCM3 expression or function, including, as examples, null mutations and mutations causing truncations.

By a “Valyl-tRNA Synthase nucleic acid sequence” is meant a nucleic acid sequence that has at least 76%, 80%, 85%, 90%, 95%, or 98% identity to the zebrafish Valyl-tRNA Synthase nucleic acid sequence of SEQ ID NO:119 over at least 519, 550, 600, 650, 750, 1000, 1500, 2000, 2500, or 2900 contiguous nucleotides. In a desirable embodiment, a “Valyl-tRNA Synthase nucleic acid sequence” is identical to the sequence of SEQ ID NO:119. Nucleic acid molecules consisting of splice variants of Valyl-tRNA Synthase nucleic acid sequences, as well as Valyl-tRNA Synthase nucleic acid sequences containing a mutation, for example, a mutation resulting from the insertion of a virus, e.g., an insertion between nucleotides 30 and 31 of the nucleic acid sequence of SEQ ID NO:119, are also included in this definition. By a “Valyl-tRNA Synthase nucleic acid sequence” is also meant a nucleic acid molecule, such as a genomic DNA, cDNA, or RNA (e.g., mRNA) molecule, that encodes Valyl-tRNA Synthase polypeptide or a zebrafish Valyl-tRNA Synthase polypeptide (e.g., SEQ ID NO:120), or a portion thereof, as defined above. A mutation in a Valyl-tRNA Synthase nucleic acid molecule can be characterized, for example, by the insertion of a retrovirus into the nucleic acid sequence, for example, using methods described herein. In addition, the invention includes mutations that result in aberrant Valyl-tRNA Synthase expression or function, including, as examples, null mutations and mutations causing truncations.

By a “40S Ribosomal Protein S5 nucleic acid sequence” is meant a nucleic acid sequence that has at least 84%, 90%, 95%, or 98% identity to the zebrafish 40S Ribosomal Protein S5 nucleic acid sequence of SEQ ID NO:121 over at least 593 or 645 nucleotides. In a desirable embodiment, a “40S Ribosomal Protein S5 nucleic acid sequence” is identical to the sequence of SEQ ID NO:121. Nucleic acid molecules consisting of splice variants of 40S Ribosomal Protein S5 nucleic acid sequences, as well as 40S Ribosomal Protein S5 nucleic acid sequences containing a mutation, for example, a mutation resulting from the insertion of a virus, e.g., an insertion between nucleotides 31 and 32 of SEQ ID NO:121, are also included in this definition. By a “40S Ribosomal Protein S5 nucleic acid sequence” is also meant a nucleic acid molecule, such as a genomic DNA, cDNA, or RNA (e.g., mRNA) molecule, that encodes 40S Ribosomal Protein S5 polypeptide or a zebrafish 40S Ribosomal Protein S5 polypeptide (e.g., SEQ ID NO:122), or a portion thereof, as defined above. A mutation in a 40S Ribosomal Protein S5 nucleic acid molecule can be characterized, for example, by the insertion of a retrovirus into the nucleic acid sequence, for example, using methods described herein. In addition, the invention includes mutations that result in aberrant 40S Ribosomal Protein S5 expression or function, including, as examples, null mutations and mutations causing truncations.

By a “TCP-1 Beta nucleic acid sequence” is meant a nucleic acid sequence that has at least 74%, 80%, 85%, 90%, 95%, or 98% identity to the zebrafish TCP-1 Beta nucleic acid sequence of SEQ ID NO:123 over at least 500, 750, 1000, or 1100 nucleotides. In a desirable embodiment, a “TCP-1 Beta nucleic acid sequence” is identical to the sequence of SEQ ID NO:123. Nucleic acid molecules consisting of splice variants of TCP-1 Beta nucleic acid sequences, as well as TCP-1 Beta nucleic acid sequences containing a mutation, for example, a mutation resulting from the insertion of a virus, e.g., an insertion between nucleotides 63 and 64 of SEQ ID NO:123, are also included in this definition. By a “TCP-1 Beta nucleic acid sequence” is also meant a nucleic acid molecule, such as a genomic DNA, cDNA, or RNA (e.g., mRNA) molecule, that encodes TCP-1 Beta polypeptide or a zebrafish TCP-1 Beta polypeptide (e.g., SEQ ID NO:124), or a portion thereof, as defined above. A mutation in a TCP-1 Beta nucleic acid molecule can be characterized, for example, by the insertion of a retrovirus into the nucleic acid sequence, for example, using methods described herein. In addition, the invention includes mutations that result in aberrant TCP-1 Beta expression or function, including, as examples, null mutations and mutations causing truncations.

By a “TCP-1 Eta nucleic acid sequence” is meant a nucleic acid sequence that has at least 79%, 85%, 90%, 95%, or 98% identity to the zebrafish TCP-1 Eta nucleic acid sequence of SEQ ID NO:125 over at least 1584, 1750, or 2000 nucleotides. In a desirable embodiment, a “TCP-1 Eta nucleic acid sequence” is identical to the sequence of SEQ ID NO:125. Nucleic acid molecules consisting of splice variants of TCP-1 Eta nucleic acid sequences, as well as TCP-1 Eta nucleic acid sequences containing a mutation, for example, a mutation resulting from the insertion of a virus, e.g., an insertion between nucleotides 32 and 33 of SEQ ID NO:125, are also included in this definition. By a “TCP-1 Eta nucleic acid sequence” is also meant a nucleic acid molecule, such as a genomic DNA, cDNA, or RNA (e.g., mRNA) molecule, that encodes TCP-1 Eta polypeptide or a zebrafish TCP-1 Eta polypeptide (e.g., SEQ ID NO:126), or a portion thereof, as defined above. A mutation in a TCP-1 Eta nucleic acid molecule can be characterized, for example, by the insertion of a retrovirus into the nucleic acid sequence, for example, using methods described herein. In addition, the invention includes mutations that result in aberrant TCP-1 Eta expression or function, including, as examples, null mutations and mutations causing truncations.

By a “Translation Elongation Factor eEF1 Alpha nucleic acid sequence” is meant a nucleic acid sequence that is identical to a zebrafish Translation Elongation Factor eEF1 Alpha nucleic acid sequence, for example, the nucleic acid sequence of SEQ ID NO:127. Nucleic acid molecules consisting of splice variants of Translation Elongation Factor eEF1 Alpha nucleic acid sequences, as well as Translation Elongation Factor eEF1 Alpha nucleic acid sequences containing a mutation, for example, a mutation resulting from the insertion of a virus, e.g., an insertion between nucleotides 60 and 61 of SEQ ID NO:127, are also included in this definition. By a “Translation Elongation Factor eEF1 Alpha nucleic acid sequence” is also meant a nucleic acid molecule, such as a genomic DNA, cDNA, or RNA (e.g., mRNA) molecule, that encodes Translation Elongation Factor eEF1 Alpha polypeptide or a zebrafish Translation Elongation Factor eEF1 Alpha polypeptide (e.g., SEQ ID NO:128), or a portion thereof, as defined above. A mutation in a Translation Elongation Factor eEF1 Alpha nucleic acid molecule can be characterized, for example, by the insertion of a retrovirus into the nucleic acid sequence, for example, using methods described herein. In addition, the invention includes mutations that result in aberrant Translation Elongation Factor eEF1 Alpha expression or function, including, as examples, null mutations and mutations causing truncations.

By a “1257 nucleic acid sequence” is meant a nucleic acid sequence that has at least 40%, 45%, 50%, 55%, 60%, 70%, 80%, 90%, 95%, or 98% identity to the zebrafish 1257 nucleic acid sequence of SEQ ID NO:129 over at least 100, 150, 200, 250, 300, 400, 500, 600, 750, 1000, 1250, 1500, 1750, or 1800 contiguous nucleotides. In a desirable embodiment, a “1257 nucleic acid sequence” is identical to the sequence of SEQ ID NO:129. Nucleic acid molecules consisting of splice variants of 1257 nucleic acid sequences, as well as 1257 nucleic acid sequences containing a mutation, for example, a mutation resulting from the insertion of a virus, e.g., an insertion at nucleotide 175 of SEQ ID NO:129, are also included in this definition. By a “1257 nucleic acid sequence” is also meant a nucleic acid molecule, such as a genomic DNA, cDNA, or RNA (e.g., mRNA) molecule, that encodes 1257 polypeptide or a zebrafish 1257 polypeptide (e.g., SEQ ID NO:130), or a portion thereof, as defined above. A mutation in a 1257 nucleic acid molecule can be characterized, for example, by the insertion of a retrovirus into the nucleic acid sequence, for example, using methods described herein. In addition, the invention includes mutations that result in aberrant 1257 expression or function, including, as examples, null mutations and mutations causing truncations.

By a “60S Ribosomal Protein L24 nucleic acid sequence” is meant a nucleic acid sequence that has at least 82%, 85%, 90%, 95%, or 98% identity to the zebrafish 60S Ribosomal Protein L24 nucleic acid sequence of SEQ ID NO:131 over at least 250, 363, 400, 500, or 565 nucleotides. In a desirable embodiment, a “60S Ribosomal Protein L24 nucleic acid sequence” is identical to the sequence of SEQ ID NO:131. Nucleic acid molecules consisting of splice variants of 60S Ribosomal Protein L24 nucleic acid sequences, as well as 60S Ribosomal Protein L24 nucleic acid sequences containing a mutation, for example, a mutation resulting from the insertion of a virus, e.g., an insertion between nucleotides 144 and 145 of the nucleic acid sequence of SEQ ID NO:131, are also included in this definition. By a “60S Ribosomal Protein L24 nucleic acid sequence” is also meant a nucleic acid molecule, such as a genomic DNA, cDNA, or RNA (e.g., mRNA) molecule, that encodes 60S Ribosomal Protein L24 polypeptide or a zebrafish 60S Ribosomal Protein L24 polypeptide (e.g., SEQ ID NO:132), or a portion thereof, as defined above. A mutation in a 60S Ribosomal Protein L24 nucleic acid molecule can be characterized, for example, by the insertion of a retrovirus into the nucleic acid sequence, for example, using methods described herein. In addition, the invention includes mutations that result in aberrant 60S Ribosomal Protein L24 expression or function, including, as examples, null mutations and mutations causing truncations.

By a “Non-Muscle Adenylosuccinate Synthase nucleic acid sequence” is meant a nucleic acid sequence that has at least 76%, 80%, 85%, 90%, 95%, or 98% identity to the zebrafish Non-Muscle Adenylosuccinate Synthase nucleic acid sequence of SEQ ID NO:133 over at least 250, 333, 400, 500, 600, 700, 800, 900, 1000, 1250, 1500, or 1700 nucleotides. In a desirable embodiment, a “Non-Muscle Adenylosuccinate Synthase nucleic acid sequence” is identical to the sequence of SEQ ID NO:133. Nucleic acid molecules consisting of splice variants of Non-Muscle Adenylosuccinate Synthase nucleic acid sequences, as well as Non-Muscle Adenylosuccinate Synthase nucleic acid sequences containing a mutation, for example, a mutation resulting from the insertion of a virus, e.g., an insertion at nucleotide 209 or between nucleotides 217 and 218 of SEQ ID NO:133, are also included in this definition. By a “Non-Muscle Adenylosuccinate Synthase nucleic acid sequence” is also meant a nucleic acid molecule, such as a genomic DNA, cDNA, or RNA (e.g., mRNA) molecule, that encodes Non-Muscle Adenylosuccinate Synthase polypeptide or a zebrafish Non-Muscle Adenylosuccinate Synthase polypeptide (e.g., SEQ ID NO:134), or a portion thereof, as defined above. A mutation in a Non-Muscle Adenylosuccinate Synthase nucleic acid molecule can be characterized, for example, by the insertion of a retrovirus into the nucleic acid sequence, for example, using methods described herein. In addition, the invention includes mutations that result in aberrant Non-Muscle Adenylosuccinate Synthase expression or function, including, as examples, null mutations and mutations causing truncations.

By a “Nuclear Cap Binding Protein Subunit 2 nucleic acid sequence” is meant a nucleic acid sequence that has at least 74%, 80%, 85%, 90%, 95%, or 98% identity to the zebrafish Nuclear Cap Binding Protein Subunit 2 nucleic acid sequence of SEQ ID NO:135 over at least 390, 500, 600, 700, or 740 nucleotides. In a desirable embodiment, a “Nuclear Cap Binding Protein Subunit 2 nucleic acid sequence” is identical to the sequence of SEQ ID NO:135. Nucleic acid molecules consisting of splice variants of Nuclear Cap Binding Protein Subunit 2 nucleic acid sequences, as well as Nuclear Cap Binding Protein Subunit 2 nucleic acid sequences containing a mutation, for example, a mutation resulting from the insertion of a virus, e.g., an insertion between nucleotides 137 and 138 of SEQ ID NO:135, are also included in this definition. By a “Nuclear Cap Binding Protein Subunit 2 nucleic acid sequence” is also meant a nucleic acid molecule, such as a genomic DNA, cDNA, or RNA (e.g., mRNA) molecule, that encodes Nuclear Cap Binding Protein Subunit 2 polypeptide or a zebrafish Nuclear Cap Binding Protein Subunit 2 polypeptide (e.g., SEQ ID NO:136), or a portion thereof, as defined above. A mutation in a Nuclear Cap Binding Protein Subunit 2 nucleic acid molecule can be characterized, for example, by the insertion of a retrovirus into the nucleic acid sequence, for example, using methods described herein. In addition, the invention includes mutations that result in aberrant Nuclear Cap Binding Protein Subunit 2 expression or function, including, as examples, null mutations and mutations causing truncations.

By a “Ornithine Decarboxylase nucleic acid sequence” is meant a nucleic acid sequence that is identical to a zebrafish Ornithine Decarboxylase nucleic acid sequence, for example, that of SEQ ID NO:137. Nucleic acid molecules consisting of splice variants of Ornithine Decarboxylase nucleic acid sequences, as well as Ornithine Decarboxylase nucleic acid sequences containing a mutation, for example, a mutation resulting from the insertion of a virus, e.g., an insertion between nucleotides 97 and 98 of SEQ ID NO:137, are also included in this definition. By a “Ornithine Decarboxylase nucleic acid sequence” is also meant a nucleic acid molecule, such as a genomic DNA, cDNA, or RNA (e.g., mRNA) molecule, that encodes Ornithine Decarboxylase polypeptide or a zebrafish Ornithine Decarboxylase polypeptide (e.g., SEQ ID NO:138), or a portion thereof, as defined above. A mutation in an Ornithine Decarboxylase nucleic acid molecule can be characterized, for example, by the insertion of a retrovirus into the nucleic acid sequence, for example, using methods described herein. In addition, the invention includes mutations that result in aberrant Ornithine Decarboxylase expression or function, including, as examples, null mutations and mutations causing truncations.

By a “Protein Phosphatase 1 Nuclear Targeting Subunit (PNUTS) nucleic acid sequence” is meant a nucleic acid sequence that has at least 76%, 80%, 85%, 90%, 95%, or 98% identity to the zebrafish Protein Phosphatase 1 Nuclear Targeting Subunit (PNUTS) nucleic acid sequence of SEQ ID NO:139 over at least 240, 300, 400, 500, 600, 700, 800, 900, 1000, 1500, or 2000 nucleotides. In a desirable embodiment, a “Protein Phosphatase 1 Nuclear Targeting Subunit (PNUTS) nucleic acid sequence” is identical to the sequence of SEQ ID NO:139. Nucleic acid molecules consisting of splice variants of Protein Phosphatase 1 Nuclear Targeting Subunit (PNUTS) nucleic acid sequences, as well as Protein Phosphatase 1 Nuclear Targeting Subunit (PNUTS) nucleic acid sequences containing a mutation, for example, a mutation resulting from the insertion of a virus, e.g., an insertion at nucleotide 303 of SEQ ID NO:139, are also included in this definition. By a “Protein Phosphatase 1 Nuclear Targeting Subunit (PNUTS) nucleic acid sequence” is also meant a nucleic acid molecule, such as a genomic DNA, cDNA, or RNA (e.g., mRNA) molecule, that encodes Protein Phosphatase 1 Nuclear Targeting Subunit (PNUTS) polypeptide or a zebrafish Protein Phosphatase 1 Nuclear Targeting Subunit (PNUTS) polypeptide (e.g., SEQ ID NO:140), or a portion thereof, as defined above. A mutation in a Protein Phosphatase 1 Nuclear Targeting Subunit (PNUTS) nucleic acid molecule can be characterized, for example, by the insertion of a retrovirus into the nucleic acid sequence, for example, using methods described herein. In addition, the invention includes mutations that result in aberrant Protein Phosphatase 1 Nuclear Targeting Subunit (PNUTS) expression or function, including, as examples, null mutations and mutations causing truncations.

By a “Mitochondrial Inner Membrane Translocating Protein (rTIM23) nucleic acid sequence” is meant a nucleic acid sequence that has at least 76%, 80%, 85%, 90%, 95%, or 98% identity to the zebrafish Mitochondrial Inner Membrane Translocating Protein (rTIM23) nucleic acid sequence of SEQ ID NO:141 over at least 250, 416, 500, 600, 700, 800, 900, 1000, or 1250 nucleic acids. In a desirable embodiment, a “Mitochondrial Inner Membrane Translocating Protein (rTIM23) nucleic acid sequence” is identical to the sequence of SEQ ID NO:141. Nucleic acid molecules consisting of splice variants of Mitochondrial Inner Membrane Translocating Protein (rTIM23) nucleic acid sequences, as well as Mitochondrial Inner Membrane Translocating Protein (rTIM23) nucleic acid sequences containing a mutation, for example, a mutation resulting from the insertion of a virus, e.g., an insertion at nucleotide 100 of SEQ ID NO:141, are also included in this definition. By a “Mitochondrial Inner Membrane Translocating Protein (rTIM23) nucleic acid sequence” is also meant a nucleic acid molecule, such as a genomic DNA, cDNA, or RNA (e.g., mRNA) molecule, that encodes Mitochondrial Inner Membrane Translocating Protein (rTIM23) polypeptide or a zebrafish Mitochondrial Inner Membrane Translocating Protein (rTIM23) polypeptide (e.g., SEQ ID NO:142), or a portion thereof, as defined above. A mutation in a Mitochondrial Inner Membrane Translocating Protein (rTIM23) nucleic acid molecule can be characterized, for example, by the insertion of a retrovirus into the nucleic acid sequence, for example, using methods described herein. In addition, the invention includes mutations that result in aberrant Mitochondrial Inner Membrane Translocating Protein (rTIM23) expression or function, including, as examples, null mutations and mutations causing truncations.

By a “1447 nucleic acid sequence” is meant a nucleic acid sequence that has at least 76%, 80%, 85%, 90%, 95%, or 98% identity to the zebrafish 1447 nucleic acid sequence of SEQ ID NO:143 over at least 500, 750, 910, 1000, 1250, 1500, 2000, 2500, or 2800 nucleic acids. In a desirable embodiment, a “1447 nucleic acid sequence” is identical to the sequence of SEQ ID NO:143. Nucleic acid molecules consisting of splice variants of 1447 nucleic acid sequences, as well as 1447 nucleic acid sequences containing a mutation, for example, a mutation resulting from the insertion of a virus, e.g., an insertion between nucleotides 227 and 228 of SEQ ID NO:143, are also included in this definition. By a “1447 nucleic acid sequence” is also meant a nucleic acid molecule, such as a genomic DNA, cDNA, or RNA (e.g., mRNA) molecule, that encodes 1447 polypeptide or a zebrafish 1447 polypeptide (e.g., SEQ ID NO:144), or a portion thereof, as defined above. A mutation in a 1447 nucleic acid molecule can be characterized, for example, by the insertion of a retrovirus into the nucleic acid sequence, for example, using methods described herein. In addition, the invention includes mutations that result in aberrant 1447 expression or function, including, as examples, null mutations and mutations causing truncations.

By an “ARS2 nucleic acid sequence” is meant a nucleic acid sequence that has at least 75%, 80%, 85%, 90%, 95%, or 98% identity to the zebrafish ARS2 nucleic acid sequence of SEQ ID NO:145 over at least 250, 500, 614, 750, 1000, 1250, 1500, 2000, or 2400 nucleic acids. In a desirable embodiment, an “ARS2 nucleic acid sequence” is identical to the sequence of SEQ ID NO:145. Nucleic acid molecules consisting of splice variants of ARS2 nucleic acid sequences, as well as ARS2 nucleic acid sequences containing a mutation, for example, a mutation resulting from the insertion of a virus, e.g., an insertion between nucleotides 103 and 104 of SEQ ID NO:143, are also included in this definition. By an “ARS2 nucleic acid sequence” is also meant a nucleic acid molecule, such as a genomic DNA, cDNA, or RNA (e.g., mRNA) molecule, that encodes ARS2 polypeptide or a zebrafish ARS2 polypeptide (e.g., SEQ ID NO:146), or a portion thereof, as defined above. A mutation in an ARS2 nucleic acid molecule can be characterized, for example, by the insertion of a retrovirus into the nucleic acid sequence, for example, using methods described herein. In addition, the invention includes mutations that result in aberrant ARS2 expression or function, including, as examples, null mutations and mutations causing truncations.

By a “Sec61 Alpha nucleic acid sequence” is meant a nucleic acid sequence that is identical to a zebrafish Sec61 Alpha nucleic acid sequence, for example, that of SEQ ID NO:147. Nucleic acid molecules consisting of splice variants of Sec61 Alpha nucleic acid sequences, as well as Sec61 Alpha nucleic acid sequences containing a mutation, for example, a mutation resulting from the insertion of a virus, e.g., an insertion between nucleotides 132 and 133 of SEQ ID NO:147, are also included in this definition. By a “Sec61 Alpha nucleic acid sequence” is also meant a nucleic acid molecule, such as a genomic DNA, cDNA, or RNA (e.g., mRNA) molecule, that encodes Sec61 alpha polypeptide or a zebrafish Sec61 alpha polypeptide (e.g., SEQ ID NO:148), or a portion thereof, as defined above. A mutation in a Sec61 Alpha nucleic acid molecule can be characterized, for example, by the insertion of a retrovirus into the nucleic acid sequence, for example, using methods described herein. In addition, the invention includes mutations that result in aberrant Sec61 Alpha expression or function, including, as examples, null mutations and mutations causing truncations.

By a “BAF53a nucleic acid sequence” is meant a nucleic acid sequence that has at least 77%, 85%, 90%, 95%, or 98% identity to the zebrafish BAF53a nucleic acid sequence of SEQ ID NO:149 over at least 500, 750, 1000, 1288, 1500, or 1800 nucleic acids. In a desirable embodiment, a “BAF53a nucleic acid sequence” is identical to the sequence of SEQ ID NO:149. Nucleic acid molecules consisting of splice variants of BAF53a nucleic acid sequences, as well as BAF53a nucleic acid sequences containing a mutation, for example, a mutation resulting from the insertion of a virus, e.g., an insertion at nucleotide 160 of SEQ ID NO:149, are also included in this definition. By a “BAF53a nucleic acid sequence” is also meant a nucleic acid molecule, such as a genomic DNA, cDNA, or RNA (e.g., mRNA) molecule, that encodes BAF53a polypeptide or a zebrafish BAF53a polypeptide (e.g., SEQ ID NO:150), or a portion thereof, as defined above. A mutation in a BAF53a nucleic acid molecule can be characterized, for example, by the insertion of a retrovirus into the nucleic acid sequence, for example, using methods described herein. In addition, the invention includes mutations that result in aberrant BAF53a expression or function, including, as examples, null mutations and mutations causing truncations.

By a “Histone Deacetylase nucleic acid sequence” is meant a nucleic acid sequence that has at least 81%, 85%, 90%, 95%, or 98% identity to the zebrafish Histone Deacetylase nucleic acid sequence of SEQ ID NO:151 over at least 500, 750, 1000, 1250, 1406, 1500, or 2000 nucleic acids. In a desirable embodiment, a “Histone Deacetylase nucleic acid sequence” is identical to the sequence of SEQ ID NO:151. Nucleic acid molecules consiting of splice variants of Histone Deacetylase nucleic acid sequences, as well as Histone Deacetylase nucleic acid sequences containing a mutation, for example, a mutation resulting from the insertion of a virus, e.g., an insertion at nucleotide 88 or between nucleotides 98 and 99 of SEQ ID NO:151, are also included in this definition. By a “Histone Deacetylase nucleic acid sequence” is also meant a nucleic acid molecule, such as a genomic DNA, cDNA, or RNA (e.g., mRNA) molecule, that encodes Histone Deacetylase polypeptide or a zebrafish Histone Deacetylase polypeptide (e.g., SEQ ID NO:152), or a portion thereof, as defined above. A mutation in a Histone Deacetylase nucleic acid molecule can be characterized, for example, by the insertion of a retrovirus into the nucleic acid sequence, for example, using methods described herein. In addition, the invention includes mutations that result in aberrant Histone Deacetylase expression or function, including, as examples, null mutations and mutations causing truncations.

By a “Fibroblast Isoform of the ADP/ATP Carrier Protein nucleic acid sequence” is meant a nucleic acid sequence that has at least 83%, 87%, 90%, 95%, or 98% identity to the zebrafish Fibroblast Isoform of the ADP/ATP Carrier Protein nucleic acid sequence of SEQ ID NO:153 over at least 500, 750, 886, 1000, or 1200 nucleic acids. In a desirable embodiment, a “Fibroblast Isoform of the ADP/ATP Carrier Protein nucleic acid sequence” is identical to the sequence of SEQ ID NO:153. Nucleic acid molecules consisting of splice variants of Fibroblast Isoform of the ADP/ATP Carrier Protein nucleic acid sequences, as well as Fibroblast Isoform of the ADP/ATP Carrier Protein nucleic acid sequences containing a mutation, for example, a mutation resulting from the insertion of a virus, e.g., an insertion between nucleotides 178 and 179 of SEQ ID NO:153, are also included in this definition. By a “Fibroblast Isoform of the ADP/ATP Carrier Protein nucleic acid sequence” is also meant a nucleic acid molecule, such as a genomic DNA, cDNA, or RNA (e.g., mRNA) molecule, that encodes Fibroblast Isoform of the ADP/ATP Carrier Protein polypeptide or a zebrafish Fibroblast Isoform of the ADP/ATP Carrier Protein polypeptide (e.g., SEQ ID NO:154), or a portion thereof, as defined above. A mutation in a Fibroblast Isoform of the ADP/ATP Carrier Protein nucleic acid molecule can be characterized, for example, by the insertion of a retrovirus into the nucleic acid sequence, for example, using methods described herein. In addition, the invention includes mutations that result in aberrant Fibroblast Isoform of the ADP/ATP Carrier Protein expression or function, including, as examples, null mutations and mutations causing truncations.

By a “TAFII-55 nucleic acid sequence” is meant a nucleic acid sequence that has at least 75%, 80%, 85%, 90%, 95%, or 98% identity to the zebrafish TAFII-55 nucleic acid sequence of SEQ ID NO:155 over at least 250, 559, 750, 900, 1000, 1250, or 1400 nucleic acids. In a desirable embodiment, a “TAFII-55 nucleic acid sequence” is identical to the sequence of SEQ ID NO:155. Nucleic acid molecules consisting of splice variants of TAFII-55 nucleic acid sequences, as well as TAFII-55 nucleic acid sequences containing a mutation, for example, a mutation resulting from the insertion of a virus, e.g., an insertion between nucleotides 107 and 108 of SEQ ID NO:155, are also included in this definition. By a “TAFII-55 nucleic acid sequence” is also meant a nucleic acid molecule, such as a genomic DNA, cDNA, or RNA (e.g., mRNA) molecule, that encodes TAFII-55 polypeptide or a zebrafish TAFII-55 polypeptide (e.g., SEQ ID NO:154), or a portion thereof, as defined above. A mutation in a TAFII-55 nucleic acid molecule can be characterized, for example, by the insertion of a retrovirus into the nucleic acid sequence, for example, using methods described herein. In addition, the invention includes mutations that result in aberrant TAFII-55 expression or function, including, as examples, null mutations and mutations causing truncations.

By an “alteration” or a “mutation” in reference to a nucleic acid sequence is meant a change in the nucleic acid sequence relative to that of a wild-type sequence. Such a change may include, for example, a substitution of one nucleotide for another, an inversion, a deletion or insertion of one or more nucleic acids, or a duplication of one or more nucleic acids. In reference to an amino acid sequence, an “alteration”, or “mutation” includes a change in the amino acid sequence relative to that of a wild-type organism. Such a change in an amino acid sequence may be, for example, a substitution of one amino acid for another, a deletion or insertion of one or more amino acids, or a duplication of one or more amino acids.

By an “alteration in level,” in reference to a nucleic acid molecule or an amino acid molecule, for example, a 904, POU2, 40S Ribosomal Protein S18, U2AF, 954, Nrp-1, Cad-1, V-ATPase alpha subunit, V-ATPase SFD subunit, V-ATPase 16 kDa proteolytic subunit, 1463, VPSP18, Pescadillo, HNF1-β/vHNF1, 60S Ribosomal Protein L35, 60S Ribosomal Protein L44, CopZ1, 215, 307, 572, 1116A, 1548, Casein Kinase 1 α, Nodal-Related or Squint, Smoothened, 429, 428, Spinster, Glypican-6 or Knypek, Ribonucleotide Reductase Protein R1 Class 1, Kinesin-Related Motor Protein EG5, 459, Wnt5 or Pipetail, Aryl Hydrocarbon Receptor Nuclear Translocator 2A, Vesicular Integral-Membrane Protein VIP 36, 299, 994, 1373, Denticleless, Ribonucleotide Reductase Protein R2, TCP-1 Alpha, Telomeric Repeat Factor 2, SIL, U1 Small Nuclear Ribonucleoprotein C, Ski Interacting Protein, 297, TCP-1 Complex Gamma Chain, Small Nuclear Ribonucleoprotein D1, DNA Polymerase Epsilon Subunit B, 821-02, 1045, 1055-01, Spliceosome Associated Protein 49, DNA Replication Licensing Factor MCM7, DEAD-Box RNA Helicase (DEAD5 or DEAD19), 1581, Cyclin A2, ISWI/SNF2, Chromosomal Assembly Protein C (XCAP-C), DNA Replication Licensing Factor MCM2, DNA Replication Licensing Factor MCM3, Valyl-tRNA Synthase, 40S Ribosomal Protein S5, TCP-1 Beta, TCP-1 Eta, Translation Elongation Factor eEF1 Alpha, 1257, 60S Ribosomal Protein L24, Non-Muscle Adenylosuccinate Synthase, Nuclear Cap Binding Protein Subunit 2, Ornithine Decarboxylase, Protein Phosphatase 1 Nuclear Targeting Subunit (PNUTS), Mitochondrial Inner Membrane Translocating Protein (rTIM23), 1447, ARS2, Sec61 Alpha, BAF53a, Histone Deacetylase, Fibroblast Isoform of the ADP/ATP Carrier Protein, or a TAFII-55 polypeptide or nucleic acid sequence is meant a change, such as an increase or a decrease in expression level of an endogenous (e.g., a naturally-occuring sequence) or heterologous nucleic acid or amino acid sequence.

Desirably, such an increase or decrease in the expression of a nucleic acid or amino acid sequence is, for example, at least 20%, 40%, 50%, 60%, 70%, or 80%. In more desirable embodiments, the decrease or increase may be, for example, 90%, 95%, or even 100%. Thus, a decrease may be a complete lack of expression of a nucleic acid or amino acid sequence. Further, an increase in the expression of a nucleic acid or amino acid sequence may be, for example, 2-fold, 3-fold, 5-fold, or even 10-fold. For instance, one can detect an alteration in the level of a nucleic acid by amplifying the sequence, or part thereof, using standard techniques such as quantitative Polymerase Chain Reaction (PCR) analysis, hybridization analysis, gel electrophoresis, Northern blots, Southern blots, and spectrophotometric assays. Alternatively, an alteration in the level of an amino acid sequence may be detected, for example, by using an antibody specific for this amino acid sequence and performing a Western blot. In addition, amino acid levels may be detected using Bradford assays and spectrophotometric assays.

By an “alteration in sequence,” in reference to a nucleic acid or amino acid sequence, for example, a 904, POU2, 40S Ribosomal Protein S18, U2AF, 954, Nrp-1, Cad-1, V-ATPase alpha subunit, V-ATPase SFD subunit, V-ATPase 16 kDa proteolytic subunit, 1463, VPSP18, Pescadillo, HNF1-β/vHNF1, 60S Ribosomal Protein L35, 60S Ribosomal Protein L44, CopZ1, 215, 307, 572, 1116A, 1548, Casein Kinase 1 α, Nodal-Related or Squint, Smoothened, 429, 428, Spinster, Glypican-6 or Knypek, Ribonucleotide Reductase Protein R1 Class 1, Kinesin-Related Motor Protein EG5, 459, Wnt5 or Pipetail, Aryl Hydrocarbon Receptor Nuclear Translocator 2A, Vesicular Integral-Membrane Protein VIP 36, 299, 994, 1373, Denticleless, Ribonucleotide Reductase Protein R2, TCP-1 Alpha, Telomeric Repeat Factor 2, SIL, U1 Small Nuclear Ribonucleoprotein C, Ski Interacting Protein, 297, TCP-1 Complex Gamma Chain, Small Nuclear Ribonucleoprotein D1, DNA Polymerase Epsilon Subunit B, 821-02, 1045, 1055-01, Spliceosome Associated Protein 49, DNA Replication Licensing Factor MCM7, DEAD-Box RNA Helicase (DEAD5 or DEAD19), 1581, Cyclin A2, ISWI/SNF2, Chromosomal Assembly Protein C (XCAP-C), DNA Replication Licensing Factor MCM2, DNA Replication Licensing Factor MCM3, Valyl-tRNA Synthase, 40S Ribosomal Protein S5, TCP-1 Beta, TCP-1 Eta, Translation Elongation Factor eEF1 Alpha, 1257, 60S Ribosomal Protein L24, Non-Muscle Adenylosuccinate Synthase, Nuclear Cap Binding Protein Subunit 2, Ornithine Decarboxylase, Protein Phosphatase 1 Nuclear Targeting Subunit (PNUTS), Mitochondrial Inner Membrane Translocating Protein (rTIM23), 1447, ARS2, Sec61 Alpha, BAF53a, Histone Deacetylase, Fibroblast Isoform of the ADP/ATP Carrier Protein, or a TAFII-55 amino acid or nucleic acid sequence is meant a change, for example, one resulting from a mutation in an endogenous (e.g., a naturally-occuring sequence) nucleic acid sequence. For example, one can detect an alteration in a nucleic acid sequence, or part thereof, by Restriction Fragment Length Polymorphism (“RFLP”) analysis and by amplifying the sequence, or a fragment thereof, using standard techniques such as the Polymerase Chain Reaction (“PCR”) and determining its sequence using standard DNA sequencing protocols. In addition, an alteration in an amino acid sequence may be detected, for example, using standard peptide sequencing protocols.

By “anti-sense,” as used herein in reference to a nucleic acid sequence, is meant a nucleic acid sequence, regardless of length, that is complementary to the coding strand or mRNA of a nucleic acid sequence. Desirably the anti-sense nucleic acid is capable of decreasing the expression or biological activity of a nucleic acid or amino acid sequence. In a desirable embodiment, the decrease in expression or biological activity is at least 10%, relative to a control, more desirably 25%, and most desirably 50% or more. The anti-sense nucleic acid may contain a modified backbone, for example, phosphorothioate, phosphorodithioate, or other modified backbones known in the art, or may contain non-natural internucleoside linkages.

By “biological activity” is meant any activity that is caused by a nucleic acid or amino acid sequence either in vivo or in vitro. For example, the biological activity of a 459 amino acid or nucleic acid sequence may be regulation of kidney development or function or regulation of cell proliferation.

By a “candidate compound” or “test compound” is meant a chemical, be it naturally-occurring or artificially-derived, that is surveyed for its ability to modulate the biological activity of a nucleic acid or amino acid molecule, by employing one of the assay methods described herein. Candidate compounds may include, for example, peptides, polypeptides, synthetic organic molecules, naturally-occurring organic molecules, nucleic acid molecules, and components thereof.

By “high stringency conditions” is meant conditions that allow hybridization comparable with the hybridization that occurs using a DNA probe of at least 300, 400, or 500 nucleotides in length, in a buffer containing, for example, 0.5 M NaHPO₄, pH 7.2, 7% SDS, 1 mM EDTA, and 1% BSA (fraction V), at a temperature of about 65° C., or a buffer containing, for example, 48% formamide, 4.8×SSC, 0.2 M Tris-Cl, pH 7.6, 1× Denhardt's solution, 10% dextran sulfate, and 0.1% SDS, at a temperature of about 42° C. (These are typical conditions for high stringency Northern or Southern hybridizations.) High stringency hybridization may also be used in numerous techniques routinely performed by molecular biologists, such as high stringency PCR, DNA sequencing, single strand conformational polymorphism analysis, and in situ hybridization. In contrast to Northern and Southern hybridizations, these techniques are usually performed with relatively short probes (e.g., usually 16 nucleotides or longer for PCR or sequencing, and 40 nucleotides or longer for in situ hybridization). The high stringency conditions used in these techniques are well known to those skilled in the art of molecular biology, and examples of them can be found, for example, in Ausubel et al. (Current Protocols in Molecular Biology, John Wiley & Sons, New York, 2000), which is hereby incorporated by reference.

The term “identity” is used herein to describe the relationship of the sequence of a particular nucleic acid molecule or polypeptide to the sequence of a reference molecule of the same type. For example, if a polypeptide or a nucleic acid molecule has the same amino acid or nucleotide residue at a given position, compared to a reference molecule to which it is aligned, there is said to be “identity” at that position. The level of sequence identity of a nucleic acid molecule or a polypeptide to a reference molecule is typically measured using sequence analysis software with the default parameters specified therein, such as the introduction of gaps to achieve an optimal alignment (e.g., EditSeq™ or MegAlign™ (DNASTAR, Inc. 1993-2001), Sequence Analysis Software Package of the Genetics Computer Group, University of Wisconsin Biotechnology Center, 1710 University Avenue, Madison, Wis. 53705, BLAST, or PILEUP/PRETTYBOX programs). These software programs match identical or similar sequences by assigning degrees of identity to various substitutions, deletions, or other modifications. Conservative substitutions typically include substitutions within the following groups: glycine, alanine, valine, isoleucine, and leucine; aspartic acid, glutamic acid, asparagine, and glutamine; serine and threonine; lysine and arginine; and phenylalanine and tyrosine.

By “isolated nucleic acid molecule” is meant a nucleic acid molecule, e.g., a DNA molecule, that is free of the nucleic acid sequence(s) which, in the naturally-occurring genome of the organism from which the nucleic acid molecule of the invention is derived, flank the nucleic acid molecule. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. The term “isolated nucleic acid molecule” also includes a recombinant DNA which is part of a hybrid gene encoding additional polypeptide sequence.

By a “kidney disorder,” as used herein is meant abnormal development, structure, or function of a kidney. Such a disorder may be congentital or it may be acquired during the life of an organism, e.g., a human. For example, a “kidney disorder” may result in the formation of fluid-filled sacs, or cysts, in a kidney. Moreover, either one or both of the kidneys may be affected by the disorder. Examples of “kidney disorders” include polycystic kidney disease, multicystic kidney disease, malformation of the kidney, Bardet-Biedl syndrome, kidney failure, acute renal failure, nephrolithiasis, congenital nephritic syndrome, kidney infection, and kidney stones.

By a “part” or “fragment,” in reference to a nucleic acid sequence is meant a stretch of 10 or more contiguous nucleic acids. In desirable embodiments, a part refers to a stretch of 20, 25, 30, 40, 50, 75, or 100 contiguous nucleic acids. In other desirable embodiments, a part is a stretch of 200, 300, 500, or 1000 contiguous nucleic acids and may include the entire coding region of a gene.

By a “part” of “fragment” in reference to an amino acid sequence is meant a stretch of 4 or more contiguous amino acids. In desirable embodiments, a part refers to a stretch of 10, 15, 25, 50, 75, or 100 contiguous amino acids. In other desirable embodiments, a part is a stretch of 200, 300, 500, or 1000 contiguous amino acids.

By “probe” or “primer” is meant a single-stranded nucleic acid sequence, for example, a DNA or RNA molecule, of defined sequence that can base pair to a second nucleic acid sequence that contains a complementary sequence (“target”). The stability of the resulting hybrid depends upon the extent of the base pairing that occurs. This stability is affected by parameters such as the degree of complementarity between the probe and target molecule, and the degree of stringency of the hybridization conditions. The degree of hybridization stringency is affected by parameters such as the temperature, salt concentration, and concentration of organic molecules, such as formamide, and is determined by methods that are well known to those skilled in the art. Probes or primers specific for a 904, POU2, 40S Ribosomal Protein S18, U2AF, 954, Nrp-1, Cad-1, V-ATPase Alpha Subunit, V-ATPase SFD subunit, V-ATPase 16 kDa proteolytic subunit, 1463, VPSP18, pescadillo, HN1F-β/vHNF1, 60S Ribosomal Protein L35, 60S Ribosomal Protein L44, CopZ1, 215, 307, 572, 1116A, 1548, Casein Kinase 1 a, Nodal-Related or Squint, Smoothened, 429, 428, Spinster, Glypican-6 or Knypek, Ribonucleotide Reductase Protein R1 Class 1, Kinesin-Related Motor Protein EG5, 459, Wnt5 or Pipetail, Aryl Hydrocarbon Receptor Nuclear Translocator 2A, Vesicular Integral-Membrane Protein VIP 36, 299, 994, 1373, Denticleless, Ribonucleotide Reductase Protein R2, TCP-1 Alpha, Telomeric Repeat Factor 2, SIL, U1 Small Nuclear Ribonucleoprotein C, Ski Interacting Protein, 297, TCP-1 Complex Gamma Chain, Small Nuclear Ribonucleoprotein D1, DNA Polymerase Epsilon Subunit B, 821-02, 1045, 1055-01, Spliceosome Associated Protein 49, DNA Replication Licensing Factor MCM7, DEAD-Box RNA Helicase (DEAD5 or DEAD19), 1581, Cyclin A2, ISWI/SNF2, Chromosomal Assembly Protein C (XCAP-C), DNA Replication Licensing Factor MCM2, DNA Replication Licensing Factor MCM3, Valyl-tRNA Synthase, 40S Ribosomal Protein S5, TCP-1 Beta, TCP-1 Eta, Translation Elongation Factor eEF1 Alpha, 1257, 60S Ribosomal Protein L24, Non-Muscle Adenylosuccinate Synthase, Nuclear Cap Binding Protein Subunit 2, Ornithine Decarboxylase, Protein Phosphatase 1 Nuclear Targeting Subunit (PNUTS), Mitochondrial Inner Membrane Translocating Protein (rTIM23), 1447, ARS2, Sec61 Alpha, BAF53a, Histone Deacetylase, Fibroblast Isoform of the ADP/ATP Carrier Protein, or a TAFII-55 nucleic acid sequences, desirably have greater than 45%, 55%, 65%, 75%, or 85% identity, in more desirable embodiments such probes have sequence identity that is at least 85-99%, and may even be identical to a fragment of or the whole length of the sequence provided in any one of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, or 157.

Probes can be detectably-labeled, either radioactively or non-radioactively, by methods that are well-known to those skilled in the art (see, for example, Ausubel et al. (Current Protocols in Molecular Biology, John Wiley & Sons, New York, 2000)). Probes can be used for methods involving nucleic acid hybridization, such as nucleic acid sequencing, nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA), and other methods that are well known to those skilled in the art.

A molecule, e.g., an oligonucleotide probe or primer, a gene or fragment thereof, a cDNA molecule, a polypeptide, or an antibody, can be said to be “detectably-labeled” if it is marked in such a way that its presence can be directly identified in a sample. Methods for detectably-labeling molecules are well known in the art and include, without limitation, radioactive labeling (e.g., with an isotope, such as ³²P or ³⁵S) and non-radioactive labeling (e.g., with a fluorescent label, such as fluorescein).

By “polypeptide” or “polypeptide fragment” is meant a chain of two or more amino acids, regardless of any post-translational modification (e.g., glycosylation or phosphorylation), constituting all or part of a naturally or non-naturally occurring polypeptide. By “post-translational modification” is meant any change to a polypeptide or polypeptide fragment during or after synthesis. Post-translational modifications can be produced naturally (such as during synthesis within a cell) or generated artificially (such as by recombinant or chemical means). A “protein” may be made up of one or more polypeptides.

By “sample” is meant a tissue biopsy, amniotic fluid, cell, blood, serum, urine, stool, or other specimen from a patient or a test subject. The sample can be analyzed to detect a mutation in, or a change in expression level of, a 904, POU2, 40S Ribosomal Protein S18, U2AF, 954, Nrp-1, Cad-1, V-ATPase Alpha Subunit, V-ATPase SFD Subunit, V-ATPase 16 kDa Proteolytic Subunit, 1463, VPSP18, Pescadillo, HNF1-β/vHNF1, 60S Ribosomal Protein L35, 60S Ribosomal Protein L44, CopZ1, 215, 307, 572, 1116A, 1548, Casein Kinase 1 α, Nodal-Related or Squint, Smoothened, 429, 428, Spinster, Glypicari-6 or Knypek, Ribonucleotide Reductase Protein R1 Class 1, Kinesin-Related Motor Protein EG5, 459, Wnt5 or Pipetail, Aryl Hydrocarbon Receptor Nuclear Translocator 2A, Vesicular Integral-Membrane Protein VIP 36, 299, 994, 1373, Denticleless, Ribonucleotide Reductase Protein R2, TCP-1 Alpha, Telomeric Repeat Factor 2, SIL, U1 Small Nuclear Ribonucleoprotein C, Ski Interacting Protein, 297, TCP-1 Complex Gamma Chain, Small Nuclear Ribonucleoprotein D1, DNA Polymerase Epsilon Subunit B, 821-02, 1045, 1055-01, Spliceosome Associated Protein 49, DNA Replication Licensing Factor MCM7, DEAD-Box RNA Helicase (DEAD5 or DEAD19), 1581, Cyclin A2, ISWI/SNF2, Chromosomal Assembly Protein C (XCAP-C), DNA Replication Licensing Factor MCM2, DNA Replication Licensing Factor MCM3, Valyl-tRNA Synthase, 40S Ribosomal Protein S5, TCP-1 Beta, TCP-1 Eta, Translation Elongation Factor eEF1 Alpha, 1257, 60S Ribosomal Protein L24, Non-Muscle Adenylosuccinate Synthase, Nuclear Cap Binding Protein Subunit 2, Ornithine Decarboxylase, Protein Phosphatase 1 Nuclear Targeting Subunit (PNUTS), Mitochondrial Inner Membrane Translocating Protein (rTIM23), 1447, ARS2, Sec61 Alpha, BAF53a, Histone Deacetylase, Fibroblast Isoform of the ADP/ATP Carrier Protein, or a TAFII-55 gene. For example, methods such as sequencing, single-strand conformational polymorphism (SSCP) analysis, or restriction fragment length polymorphism (RFLP) analysis of PCR products derived from a patient sample can be used to detect a mutation in an above-listed gene; ELISA can be used to measure levels of a polypeptide encoded by an above-listed gene; and PCR can be used to measure the level of an above-listed gene or nucleic acid sequence.

An antibody is said to “specifically bind” to a polypeptide if it recognizes and binds to the polypeptide (e.g., a 904, POU2, 40S Ribosomal Protein S18, U2AF, 954, Nrp-1, Cad-1, V-ATPase Alpha Subunit, V-ATPase SFD Subunit, V-ATPase 16 kDa Proteolytic Subunit, 1463, VPSP18, Pescadillo, HNF1-β/vHNF1, 60S Ribosomal Protein L35, 60S Ribosomal Protein L44, CopZ1, 215, 307, 572, 1116A, 1548, Casein Kinase 1 α, Nodal-Related or Squint, Smoothened, 429, 428, Spinster, Glypican-6 Knypek, Ribonucleotide Reductase Protein R1 Class 1, Kinesin-Related Motor Protein EG5, 459, WNT5 or Pipetail, Aryl Hydrocarbon Receptor Nuclear Translocator 2A, Vesicular Integral-Membrane Protein VIP 36, 299, 994, 1373, Denticleless, Ribonucleotide Reductase Protein R2, TCP-1 Alpha, Telomeric Repeat Factor 2, SIL, U1 Small Nuclear Ribonucleoprotein C, Ski Interacting Protein, 297, TCP-1 Complex Gamma Chain, Small Nuclear Ribonucleoprotein D1, DNA Polymerase Epsilon Subunit B, 821-02, 1045, 1055-01, Spliceosome Associated Protein 49, DNA Replication Licensing Factor MCM7, DEAD-Box RNA Helicase (DEAD5 or DEAD19), 1581, Cyclin A2, ISWI/SNF2, Chromosomal Assembly Protein C (XCAP-C), DNA Replication Licensing Factor MCM2, DNA Replication Licensing Factor MCM3, Valyl-tRNA Synthase, 40S Ribosomal Protein S5, TCP-1 Beta, TCP-1 Eta, Translation Elongation Factor eEF1 Alpha, 1257, 60S Ribosomal Protein L24, Non-Muscle Adenylosuccinate Synthase, Nuclear Cap Binding Protein Subunit 2, Ornithine Decarboxylase, Protein Phosphatase 1 Nuclear Targeting Subunit (PNUTS), Mitochondrial Inner Membrane Translocating Protein (rTIM23), 1447, ARS2, Sec61 Alpha, BAF53a, Histone Deacetylase, Fibroblast Isoform of the ADP/ATP Carrier Protein, or a TAFII-55 polypeptide), but does not substantially recognize and bind to other, molecules in a sample, e.g., a biological sample that naturally includes the polypeptide.

A nucleic acid sequence or polypeptide is said to be “substantially identical” to a reference molecule if it exhibits, over its entire length, at least 51%, desirably at least 55%, 60%, or 65%, and in more desirable embodiments 75%, 85%, 90%, 95%, 98%, or 99% identity to the sequence of the reference molecule. For polypeptides, the length of comparison sequences is at least 16 amino acids, desirably at least 20, 30, 40, 50, 75, or 100 amino acids, and in more desirable embodiments at least 125, 150, 175, 200, 250, 300, 400, 500, 600, 700, 800, or 1000 amino acids. For nucleic acid sequences, the length of comparison sequences is at least 50 nucleotides, desirably at least 60, 90, 120, 150, 225, or 300 nucleotides, and in more desirable embodiments at least 375, 450, 525, 600, 750, 900, 1200, 1500, 2100, 2400, 2700, or 3000 nucleotides.

By a “substantially pure polypeptide” or “isolated polypeptide” is meant a polypeptide (or a fragment thereof) that has been separated from proteins and organic molecules that naturally accompany it. Typically, a polypeptide is substantially pure when it is at least 60%, by weight, free from the proteins and naturally-occurring organic molecules with which it is naturally associated. Preferably, the polypeptide is a 904, POU2, 40S Ribosomal Protein S18, U2AF, 954, Nrp-1, Cad-1, V-ATPase Alpha Subunit, V-ATPase SFD Subunit, V-ATPase 16 kDa Proteolytic Subunit, 1463, VPSP18, Pescadillo, HNF1-β/vHNF1, 60S Ribosomal Protein L35, 60S Ribosomal Protein L44, CopZ1, 215, 307, 572, 1116A, 1548, Casein Kinase 1 a, Nodal-Related or Squint, Smoothened, 429, 428, Spinster, Glypican-6 or Knypek, Ribonucleotide Reductase Protein R1 Class 1, Kinesin-Related Motor Protein EG5, 459, Wnt5 or Pipetail, Aryl Hydrocarbon Receptor Nuclear Translocator 2A, Vesicular Integral-Membrane Protein VIP 36, 299, 994, 1373, Denticleless, Ribonucleotide Reductase Protein R2, TCP-1 Alpha, Telomeric Repeat Factor 2, SIL, U1 Small Nuclear Ribonucleoprotein C, Ski Interacting Protein, 297, TCP-1 Complex Gamma Chain, Small Nuclear Ribonucleoprotein D1, DNA Polymerase Epsilon Subunit B, 821-02, 1045, 1055-01, Spliceosome Associated Protein 49, DNA Replication Licensing Factor MCM7, DEAD-Box RNA Helicase (DEAD5 or DEAD19), 1581, Cyclin A2, ISWI/SNF2, Chromosomal Assembly Protein C (XCAP-C), DNA Replication Licensing Factor MCM2, DNA Replication Licensing Factor MCM3, Valyl-tRNA Synthase, 40S Ribosomal Protein S5, TCP-1 Beta, TCP-1 Eta, Translation Elongation Factor eEF1 Alpha, 1257, 60S Ribosomal Protein L24, Non-Muscle Adenylosuccinate Synthase, Nuclear Cap Binding Protein Subunit 2, Ornithine Decarboxylase, Protein Phosphatase 1 Nuclear Targeting Subunit (PNUTS), Mitochondrial Inner Membrane Translocating Protein (rTIM23), 1447, ARS2, Sec61 Alpha, BAF53a, Histone Deacetylase, Fibroblast Isoform of the ADP/ATP Carrier Protein, or a TAFII-55 polypeptide that is at least 75%, in a more desirable embodiment at least 90%, and in another desirable embodiment at least 99%, by weight, pure. A substantially pure or isolated 904, POU2, 40S Ribosomal Protein S18, U2AF, 954, Nrp-1, Cad-1, V-ATPase Alpha Subunit, V-ATPase SFD subunit, V-ATPase 16 kDa Proteolytic Subunit, 1463, VPSP18, Pescadillo, HNF1-β/vHNF1, 60S Ribosomal Protein L35, 60S Ribosomal Protein L44, CopZ1, 215, 307, 572, 1116A, 1548, Casein Kinase 1 α, Nodal-Related or Squint, Smoothened, 429, 428, Spinster, Glypican-6 or Knypek Ribonucleotide Reductase Protein R1 Class 1, Kinesin-Related Motor Protein EG5, 459, Wnt5 or Pipetail, Aryl Hydrocarbon Receptor Nuclear Translocator 2A, Vesicular Integral-Membrane Protein VIP 36, 299, 994, 1373, Denticleless, Ribonucleotide Reductase Protein R2, TCP-1 Alpha, Telomeric Repeat Factor 2, SIL, U1 Small Nuclear Ribonucleoprotein C, Ski Interacting Protein, 297, TCP-1 Complex Gamma Chain, Small Nuclear Ribonucleoprotein D1, DNA Polymerase Epsilon Subunit B, 821-02, 1045, 1055-01, Spliceosome Associated Protein 49, DNA Replication Licensing Factor MCM7, DEAD-Box RNA Helicase (DEAD5 or DEAD19), 1581, Cyclin A2, ISWI/SNF2, Chromosomal Assembly Protein C (XCAP-C), DNA Replication Licensing Factor MCM2, DNA Replication Licensing Factor MCM3, Valyl-tRNA Synthase, 40S Ribosomal Protein S5, TCP-1 Beta, TCP-1 Eta, Translation Elongation Factor eEF1 Alpha, 1257, 60S Ribosomal Protein L24, Non-Muscle Adenylosuccinate Synthase, Nuclear Cap Binding Protein Subunit 2, Ornithine Decarboxylase, Protein Phosphatase 1 Nuclear Targeting Subunit (PNUTS), Mitochondrial Inner Membrane Translocating Protein (rTIM23), 1447, ARS2, Sec61 Alpha, BAF53a, Histone Deacetylase, Fibroblast Isoform of the ADP/ATP Carrier Protein, or TAFII-55 polypeptide can be obtained, for example, by extraction from a natural source (e.g., zebrafish or mammalian tissue), by expression of a recombinant nucleic acid sequence encoding an above-listed polypeptide, or by chemical synthesis. Purity can be measured by any appropriate method, e.g., by column chromatography, polyacrylamide gel electrophoresis, or HPLC analysis.

A polypeptide is substantially free of naturally associated components when it is separated from those proteins and organic molecules that accompany it in its natural state. Thus, a protein that is chemically synthesized or produced in a cellular system that is different from the cell in which it is naturally produced is substantially free from its naturally associated components. Accordingly, substantially pure polypeptides not only include those that are derived from eukaryotic organisms, but also those synthesized in E. coli or other prokaryotes.

By “transgene” is meant any piece of DNA which is inserted by artifice into a cell and typically becomes part of the genome of the organism which develops from that cell. Such a transgene may include a gene which is partly or entirely heterologous (i.e., foreign) to the transgenic organism, or may represent a gene homologous to an endogenous gene of the organism.

By “transgenic” is meant any cell which includes a DNA sequence which is inserted by artifice into a cell and becomes part of the genome of the organism which develops from that cell. As used herein, the transgenic organisms are generally transgenic vertebrates, such as, zebrafish, mice, and rats, and the DNA (transgene) is inserted by artifice into the nuclear genome.

### BRIEF DESCRIPTION OF THE DRAWINGS

FIG. 1 is a schematic diagram of the protocol for the large-scale mutagenesis screen.

FIG. 2 shows Southern Blots of eight fish from two different F1 families (FIGS. 2A and 2B).

FIG. 3 is a schematic diagram of the structure of the provirus along with the position of the Southern blot probes and PCR primers.

FIG. 4A is a scanned image of four-day old zebrafish embryos, lateral view. A wild-type embryo is shown at the top, and an embryo containing a mutation in the 904 nucleic acid sequence is shown at the bottom.

FIG. 4B is a scanned image of three-day old zebrafish embryos, ventral view. A wild-type embryo is shown at the top, and an embryo containing a mutation in the 904 nucleic acid sequence is shown at the bottom.

FIG. 5 is a scanned image of three-day old zebrafish embryos. A wild-type embryo is shown at the top and an embryo containing a mutation in the POU2 nucleic acid sequence is shown at the bottom of the panel.

FIG. 6A is a scanned image of two-day old zebrafish embryos. A wild type embryo is at the top and an embryo containing a mutation in the 40S Ribosomal Protein S18 nucleic acid sequence is at the bottom.

FIG. 6B is a scanned image of four two-day old zebrafish embryos. The embryo on the right is wild-type and the other three embros contain a mutation in the 40S ribosomal protein S18 nucleic acid sequence.

FIG. 7A is a scanned image of four two-day old zebrafish embryos, lateral views. A wild-type embryo is shown at the top. Embryos containing a mutation in the U2AF nucleic acid sequence are shown below.

FIG. 7B is a scanned image of a two-day old zebrafish embryo, lateral view. This embryo contains a mutation in the U2AF nucleic acid sequence.

FIG. 8A is a scanned image of four-day old zebrafish embryos, ventral view. A wild-type embryo is shown at the top. The embryo at the bottom of the panel contains a mutation in the 954 nucleic acid sequence.

FIG. 8B is a scanned image of four-day old zebrafish embryos, lateral view. The embryo at the bottom of the panel contains a mutation in the 954 nucleic acid sequence.

FIG. 9A is a scanned image of five-day old zebrafish embryos, lateral view. The embryo at the bottom of the panel contains a mutation in the Nrp-1 gene.

FIG. 9B is a scanned image of five day old zebrafish embryos stained with Alcian blue, lateral view. The embryo at the bottom of the panel contains a mutation in the Nrp-1 nucleic acid sequence.

FIG. 10 is a scanned image of two-day old zebrafish embryos, lateral view. The embryo at the bottom of the panel contains a mutation in the Cad-1 gene. Cad-1 is a caudal homeobox zinc finger homolog.

FIG. 11A is a scanned image of a five-day old zebrafish embryo that contains a mutation in the V-ATPase Alpha Subunit nucleic acid sequence, dorsal view.

FIG. 11B is a scanned image of a five-day old wild-type zebrafish embryo, dorsal view.

FIG. 12A is a scanned image of three four-day old zebrafish embryos, lateral view. The embryo at the top of the panel is wild-type. The other embryos contain mutations in the V-ATPase SFD Subunit nucleic acid sequence.

FIG. 12B is a scanned image of a four-day old zebrafish embryo, lateral view. This embryo contains a mutation in the V-ATPase SFD Subunit nucleic acid sequence and shows brain necrosis.

FIG. 13A is a scanned image of two-day old zebrafish embryos, lateral view. The embryo at the top of the panel is wild-type. The embryo at the bottom of the panel contains a mutation in the V-ATPase 16 kDa Proteolytic Subunit nucleic acid sequence.

FIG. 13B is a scanned image of three-day old zebrafish embryos, lateral views. The embryo at the top of the panel is wild-type. The embryo at the bottom of the panel contains a mutation in the V-ATPase16 kDa Proteolytic Subunit nucleic acid sequence.

FIG. 14A is a scanned image of two-day old zebrafish embryos, lateral views. The embryo at the top of the panel is wild-type. The embryo at the bottom of the panel contains a mutation in the 1463 nucleic acid sequence which is a CD36-like Transmembrane Receptor.

FIG. 14B is a scanned image of six one-day old zebrafish embryos. The embryo in the upper-left hand corner is wild-type. The other embryos contain mutations in the 1463 nucleic acid sequence which is a CD36-Like Transmembrane Receptor.

FIG. 15A is a scanned image of three five-day old embryos, lateral views. The embryo at the top of the panel is wild-type. The other embryos contain mutations in the VPSP18 nucleic acid sequence.

FIG. 15B is a scanned image of five-day old embryos, lateral views. The embryo at the top of the panel is wild-type. The other embryos contain mutations in the VPSP18 nucleic acid sequence.

FIG. 16A is a scanned image of a four-day old zebrafish embryo. This embryo contains a mutation in the HNF1-β/vHNF1 gene. The mesonephros have ballooned out, and there is a bulge in a duct at chevron eight.

FIG. 16B is a scanned image of a four-day old zebrafish embryo. This embryo contains a mutation in the HNF1-β/vHNF1 gene.

FIG. 17A is a scanned image of a two-day old zebrafish embryo. This embryo contains a mutation in the 60S Ribosomal Protein L35 gene and has abnormal somites.

FIG. 17B is a scanned image of two day old zebrafish embryos. The embryo at the top of the panel is wild type. The embryo at the bottom of the panel contains a mutation in the 60S Ribosomal Protein L35 nucleic acid sequence.

FIG. 18A is a scanned image of a two-day old wild-type zebrafish embryo, lateral view.

FIG. 18B is a scanned image of a two-day old zebrafish embryo, lateral view. This embryo contains a mutation in the 60S Ribosomal Protein L44 nucleic acid sequence.

FIG. 19A is a scanned image of a three-day old wild-type zebrafish embryo, lateral view.

FIG. 19B is a scanned image of a three-day old zebrafish embryo, lateral view. This embryo contains a mutation in the CopZ1 nucleic acid sequence.

FIG. 20 is a scanned image of three-day old zebrafish embryos. The embryo at the top is wild-type. The other embryos contain mutations in the 215 nucleic acid sequence that encodes an ATP-dependent RNA helicase.

FIG. 21A is a scanned image of six-day old zebrafish embryos stained with Alcian blue, ventral views. The embryo at the top of the panel is wild-type, the embryo at the bottom of the panel contains a mutation in the 307 nucleic acid sequence which encodes Beta-1,3-Glucuronyltransferase.

FIG. 21B is a scanned image of six-day old zebrafish embryos stained with Alcian blue, lateral views. The embryo at the top is wild-type. The embryo at the bottom contain a mutation in the 307 nucleic acid sequence which encodes a Beta-1,3-Glucuronyltransferase.

FIG. 22A is a scanned image of four-day old zebrafish embryos, lateral views. The embryo at the top of the panel is wild-type. The embryo at the bottom of the panel contains a mutation in the 572 nucleic acid sequence.

FIG. 22B is a scanned image of a four-day old zebrafish embryo, ventral view. The embryo at the top of the panel is wild-type. The embryo at the bottom of the panel contains a mutation in the 572 nucleic acid sequence.

FIG. 23A is a scanned image of a six-day old wild-type zebrafish embryo, lateral view.

FIG. 23B is a scanned image of a six-day old zebrafish embryo, lateral view. The embryo contains a mutation in the 1116A nucleic acid sequence.

FIG. 24A is a scanned image of five-day old zebrafish embryos, lateral views. The embryo at the top of the panel is wild-type. The two embryos below it contain mutations in the 1548 nucleic acid sequence.

FIG. 24B is a scanned image of five-day old zebrafish embryos, ventral views. The embryo at the top of the panel is wild-type. The two embryos below it contain mutations in the 1548 nucleic acid sequence.

FIG. 25A is a scanned image of four-day old zebrafish embryos stained with Alcian blue, ventral views. The embryo at the top of the panel is wild-type. The embryo at the bottom of the panel contains a mutation in Casein Kinasel a nucleic acid sequence that has been identified as a Casein Kinase 1 a isoform.

FIG. 25B is a scanned image of three four-day old zebrafish embryos, ventral view. The embryos below contain mutations in the Casein Kinase 1 a nucleic acid sequence.

FIG. 26 is a scanned image of a five-day old zebrafish embryo, ventral view. The embryo contains a mutation in the Nodal-related (Squint) nucleic acid sequence.

FIG. 27A is a scanned image of three-day old zebrafish embryos, ventral views. The embryo at the top of the panel is wild-type. The embryo at the bottom of the panel contains a mutation in the Smoothened nucleic acid sequence.

FIG. 27B is a scanned image of two-day old zebrafish embryos, lateral views. The embryo at the top of the panel is wild-type. The embryo at the bottom of the panel contains a mutation in the Smoothened nucleic acid sequence.

FIG. 28A is a scanned image of six-day old zebrafish embryos, lateral views. The embryo at the upper-lefthand corner of the panel is wild-type. The other embryos in the panel contain mutations in the 429 nucleic acid sequence.

FIG. 28B is a scanned image of a six-day old zebrafish embryo, lateral view. This embryo contains a mutation in the 429 nucleic acid sequence, and displays a defect in the lower jaw.

FIG. 29A is a scanned image of a four-day old wild-type zebrafish embryo, shown in indirect light.

FIG. 29B is a scanned image of a four-day old 428 mutant embryo under indirect light.

FIG. 30 is a scanned image of one-day old zebrafish embryos, lateral posterior views. The embryo at the top of the panel is wild-type. The embryo at the bottom of the panel contains a mutation in the Glypican-6 or Knypek nucleic acid sequence.

FIG. 31A is a scanned image of six-day old zebrafish embryos, ventral views. The embryo at the top of the panel is wild-type. The embryo at the bottom of the panel contains a mutation in the nucleic acid sequence that encodes Ribonucleotide Reductase Protein R1 Class 1.

FIG. 31B is a scanned image of one-day old zebrafish embryos, lateral views. The embryo at the top of the panel is wild-type. The embryo at the bottom of the panel contains a mutation in the nucleic acid sequence that encodes Ribonucleotide Reductase Protein R1 Class 1.

FIG. 32 is a scanned image of three-day old zebrafish embryos, dorsal views. The embryo at the top of the panel is wild-type. The embryo at the bottom of the panel contains a mutation in the Kinesin-Related Motor Protein EGS nucleic acid sequence.

FIG. 33A is a scanned image of one-day old zebrafish embryos, lateral views. The embryo at the top of the panel is wild-type. The embryo at the bottom of the panel contains a mutation in the 459 nucleic acid sequence.

FIG. 33B is a scanned image of two-day old zebrafish embryos, lateral views. The embryo at the top of the panel is wild-type. The embryos at the bottom of the panel contain a mutation in the 459 nucleic acid sequence.

FIG. 34A is a scanned image of three-day old zebrafish embryos, lateral views. The embryo at the top of the panel is wild-type. The embryos at the bottom of the panel contain mutations in the Wnt5 (Pipetail) nucleic acid sequence.

FIG. 34B is a scanned image of three-day old zebrafish embryos, dorsal views. The embryo at the top of the panel is wild-type. The embryo at the bottom of the panel contains a mutation in the Wnt5 (Pipetail) nucleic acid sequence.

FIG. 35 is a scanned image of five-day old zebrafish embryos. A wild-type embryo is shown at the top and an embryo containing a mutation in the Aryl Hydrocarbon Receptor Nuclear Translocator 2A nucleic acid sequence is shown at the bottom of the panel.

FIG. 36 is a scanned image of five-day old zebrafish embryos. A wild-type embryo is shown at the top and two embryos containing a mutation in the Vesicular Integral Membrane Protein (VIP 36) nucleic acid sequence are shown at the bottom of the panel.

FIG. 37A is a scanned image of four-day old zebrafish embryos, ventral views. The embryo at the left of the panel is wild-type. The embryo at the right of the panel contains a mutation in the 299 nucleic acid sequence.

FIG. 37B is a scanned image of four-day old zebrafish embryos stained with Alcian blue, ventral views. The embryo at the left of the panel is wild-type. The embryo at the right of the panel contains a mutation in the 299 nucleic acid sequence. The mutant displays defects that include no jaw and branchial arches, as well as small fins.

FIG. 38A is a scanned image of a four-day old wild-type zebrafish embryo, lateral view.

FIG. 38B is a scanned image of a four-day old zebrafish embryo, lateral view, containing a mutation in the 994 nucleic acid sequence.

FIG. 39A is a scanned image of a two-day old wild-type zebrafish embryo, lateral view.

FIG. 39B is a scanned image of a two-day old zebrafish embryo, lateral view, containing a mutation in the 1373 nucleic acid sequence

FIG. 40A is a scanned image of two-day old zebrafish embryos, lateral views. A wild-type embryo is shown at the top of the panel, and an embryo containing a mutation in the Denticleless nucleic acid sequence is shown at the bottom of the panel.

FIG. 40B is a scanned image of a two-day old zebrafish embryo, lateral view, containing a mutation in the Denticleless nucleic acid sequence.

FIG. 41A is a scanned image of two-day old zebrafish embryos, lateral view. A wild-type embryo is shown at the top of the panel, and an embryo containing a mutation in the Ribonucleoside Reductase Protein R2 nucleic acid sequence is shown at the bottom of the panel.

FIG. 41B is a scanned image of a two-day old zebrafish embryo, lateral posterior view. The posterior of a wild-type embryo is shown at the top of the panel, and an embryo containing a mutation in the Ribonucleotide Reductase Protein R2 nucleic acid sequence is shown at the bottom of the panel.

FIG. 42A is a scanned image of a four-day old zebrafish embryo, lateral view. This embryo, containing a mutation in the TCP-1 Alpha nucleic acid sequence, displays a small head and heart edema.

FIG. 42B is a scanned image of a wild-type four-day old zebrafish embryo, lateral view.

FIG. 43 is a scanned image of two-day old zebrafish embryos, lateral views. A wild-type embryo is shown at the top of the panel, and an embryo containing a mutation in the Telomeric Repeat Factor 2 nucleic acid sequence is shown at the bottom of the panel.

FIG. 44 is a scanned image of three-day old zebrafish embryos. A wild-type embryo is shown at the top and an embryo containing a mutation in the SIL nucleic acid sequence is shown at the bottom of the panel.

FIG. 45A is a scanned image of two-day old zebrafish embryos, lateral view of the midbody. A wild-type embryo is shown at the top of the panel, and an embryo containing a mutation in the U1 Small Nuclear Ribonucleoprotein C nucleic acid sequence is shown at the bottom of the panel.

FIG. 45B is a scanned image of two-day old zebrafish embryo, lateral view. A wild-type embryo is shown at the top of the panel, and an embryo containing a mutation in the U1 Small Nuclear Ribonucleoprotein C nucleic acid sequence is shown at the bottom of the panel.

FIG. 46A is a scanned image of a one-day old zebrafish embryo, lateral view. A wild-type embryo is shown at the top of the panel, note that the mid/hind-brain barrier is obvious, and brain structures clearly visible.

FIG. 46B is a scanned image of a one-day old zebrafish embryo, lateral view, with a mutation in the Ski Interacting Protein (SKIP) nucleic acid sequence. This mutant embryo has a small head due to extensive brain necrosis.

FIG. 47A is a scanned image of three-day old zebrafish embryos, lateral views. An embryo containing a mutation in the 297 nucleic acid sequence is shown at the top of the panel, note the flattened head, brain containing yellow debris, and large yolk sac. A wild-type embryo is shown at the bottom of the panel.

FIG. 47B is a scanned image of three-day old zebrafish embryos, dorsal views. A wild-type embryo is shown at the top of the panel, and an embryo containing a mutation in the 297 nucleic acid sequence is shown at the bottom of the panel. The branchial arches appear abnormal.

FIG. 48 is a scanned image of three-day old zebrafish embryos, lateral views. A wild-type embryo is shown at the top of the panel, and an embryo containing a mutation in the TCP-1 Complex Gamma Chain nucleic acid sequence is shown at the bottom of the panel.

FIG. 49A is a scanned image of a two-day old zebrafish embryo, lateral view. A wild-type embryo is shown.

FIG. 49B is a scanned image of a two-day old zebrafish embryo, lateral view, containing a mutation in the nucleic acid sequence that encodes the Small Nuclear Ribonucleoprotein D1.

FIG. 50 is a scanned image of three-day old zebrafish embryos. A wild-type embryo is shown at the top and an embryo containing a mutation in the DNA Polymerase Epsilon Subunit B nucleic acid sequence is shown at the bottom of the panel.

FIG. 51 is a series of scanned images of zebrafish embryos containing a mutation in an 821-02 nucleic acid sequence, lateral views. FIG. 51A shows a one-day old embryo and FIG. 51B shows the posterior of two-day old embryos.

FIG. 52 is a scanned image of one-day old zebrafish embryos. A wild-type embryo is shown at the top-left corner of this panel, the other three embryos contain a mutation in the 1045 nucleic acid sequence.

FIG. 53A is a scanned image of three-day old zebrafish embryos, lateral views. A wild-type embryo is shown at the top of the panel, and embryos containing mutations in the 1055-1 nucleic acid sequence are shown below. The 1055-1 gene encodes a MAK16 homolog.

FIG. 53B is a scanned image of three-day old zebrafish embryos, lateral views. A wild-type embryo is shown at the top of the panel, and an embryo containing a mutation in the 1055-1 nucleic acid sequence is shown below. The 1055-1 gene encodes a MAK16 homolog.

FIG. 54A is a scanned image of a two-day old zebrafish embryo, lateral view. A wild-type embryo is shown.

FIG. 54B is a scanned image of a two-day old zebrafish embryo, lateral view, containing a mutation in the Spliceosome Associated Protein 49 nucleic acid.

FIG. 55 is a scanned image of three-day old zebrafish embryos, lateral views. A wild-type embryo is shown at the top of the panel, and an embryo containing a mutation in the DNA Replication Licensing Factor MCM7 nucleic acid sequence is shown at the bottom of the panel.

FIG. 56A is a scanned image of a one-day old wild-type zebrafish embryo, lateral view.

FIG. 56B is a scanned image of a one-day old zebrafish embryo, lateral view. This embryo contains a mutation in the Dead-Box RNA Helicase (DEAD5 or DEAD19) nucleic acid sequence.

FIG. 57A is a scanned image of one-day old zebrafish embryos, lateral views. A wild-type embryo is shown in the left-hand panel, and an embryo containing a mutation in the 1581 nucleic acid sequence is shown in the right-hand panel.

FIG. 57B is a scanned image of one-day old zebrafish embryos, ventral views. A wild-type embryo is shown in the left-hand panel, and an embryo containing a mutation in the 1581 nucleic acid sequence is shown in the right-hand panel.

FIG. 58 is a scanned image of four-day old zebrafish embryos, lateral views. A wild-type embryo is shown at the top of the panel, and embryos containing mutations in the Cyclin A2 nucleic acid sequence are shown below.

FIG. 59 is a scanned image of two-day old zebrafish embryos. A wild-type embryo is shown at the top and two embryos containing a mutation in the ISWI/SNF nucleic acid sequence are shown at the bottom of the panel.

FIG. 60 is a scanned image of four-day old zebrafish embryos, lateral views. A wild-type embryo is show at the top of the panel and an embryo containing a mutation in the XCAP-C nucleic acid sequence is shown at the bottom.

FIG. 61A is a scanned image of three-day old zebrafish embryos, dorsolateral views. A wild-type embryo is shown at the top of the panel, and an embryo containing a mutation in the DNA Replication Licensing Factor MCM2 nucleic acid sequence is shown at the bottom of the panel.

FIG. 61B is a scanned image of four-day old zebrafish embryos, lateral views. A wild-type embryo is shown at the top of the panel, and an embryo containing a mutation in the DNA Replication Licensing Factor MCM2 nucleic acid sequence is shown at the bottom of the panel.

FIG. 62A is a scanned image of a four-day old zebrafish embryo, lateral view. An embryo containing a mutation in the DNA Replication Licensing Factor MCM3 nucleic acid sequence is shown.

FIG. 62B is a scanned image of a four-day old wild-type zebrafish embryo, lateral view.

FIG. 63A is a scanned image of three-day old zebrafish embryos, ventral views. A wild-type embryo is shown at the top of the panel, and an embryo containing a mutation in the Valyl-tRNA Synthase nucleic acid sequence is shown at the bottom of the panel. Note the delay in jaw development displayed by the mutant embryo.

FIG. 63B is a scanned image of four-day old zebrafish embryos, ventral views, stained with Alcian blue. A wild-type embryo is shown at the top of the panel, and an embryo containing a mutation in the Valyl-tRNA Synthase nucleic acid sequence is shown at the bottom of the panel.

FIG. 64A is a scanned image of a two-day old wild-type zebrafish embryo, lateral view.

FIG. 64B is a scanned image of a two-day old zebrafish embryo, lateral view. An embryo containing a mutation in the 40S Ribosomal Protein S5 nucleic acid sequence is shown.

FIG. 65A is a scanned image of five-day old zebrafish embryos stained with Alcian blue, ventral views. A wild-type embryo is shown at the top of the panel, and an embryo containing a mutation in the TCP-1 Beta nucleic acid sequence is shown at the bottom of the panel.

FIG. 65B is a scanned image of four-day old zebrafish embryos, ventral views. A wild-type embryo is shown at the top of the panel, and an embryo containing a mutation in the TCP-1 Beta nucleic acid sequence is shown at the bottom of the panel.

FIG. 66A is a scanned image of three-day old zebrafish embryos, dorsal views. A wild-type embryo is shown at the top of the panel, and an embryo containing a mutation in the TCP-1 Eta nucleic acid sequence is shown at the bottom of the panel.

FIG. 66B is a scanned image of five-day old zebrafish embryos, lateral views. This image shows the muscles of a wild-type embryo at the top of the panel, and a mutant embryo containing a mutation in the TCP-1 Eta nucleic acid sequence is shown at the bottom of the panel.

FIG. 67 is a series of scanned images of three-day old zebrafish embryos. A lateral view of a wild-type embryo is shown in FIG. 67A, a lateral view of an embryo containing a mutation in the Translation Elongation Factor eEF1 Alpha nucleic acid sequence is shown in FIG. 67B, and ventral views of a wild-type (top) and Translation Elongation Factor eEF1 Alpha mutant (bottom) embryos are shown in FIG. 67C.

FIG. 68A is a scanned image of a five-day old zebrafish embryo, ventral view. An embryo containing a mutation in the 1257 nucleic acid sequence is shown.

FIG. 68B is a scanned image of a five-day old zebrafish embryo, ventral view.

FIG. 69A is a scanned image of three-day old zebrafish embryos, lateral views. A wild-type embryo is shown at the top of the panel, and an embryo containing a mutation in the 60S Ribosomal Protein L24 nucleic acid sequence is shown at the bottom of the panel.

FIG. 69B is a scanned image of three-day old zebrafish embryos, lateral views. A wild-type embryo is shown at the top of the panel, and an embryo containing a mutation in the 60S Ribosomal Protein L24 nucleic acid sequence is shown at the bottom of the panel.

FIG. 70A is a scanned image of three-day old zebrafish embryos, ventral views. A wild-type embryo is shown at the top of the panel, and an embryo containing a mutation in the Non-Muscle Adenylosuccinate Synthase nucleic acid sequence is shown at the bottom of the panel.

FIG. 70B is a scanned image of four-day old zebrafish embryos, ventral views. A wild-type embryo is shown at the top of the panel, and an embryo containing a mutation in the Non-Muscle Adenylosuccinate Synthase nucleic acid sequence is shown at the bottom of the panel.

FIG. 71A is a scanned image of four-day old zebrafish embryos, lateral views. A wild-type embryo is shown at the top of the panel, and embryos containing mutations in the Nuclear-Cap Binding Protein Subunit 2 nucleic acid sequence are shown below it.

FIG. 71B is a scanned image of four-day old zebrafish embryos, lateral views. A wild-type embryo is shown at the top of the panel, and an embryo containing a mutation in the Nuclear-Cap Binding Protein Subunit 2 nucleic acid sequence is shown at the bottom of the panel.

FIG. 72 is a scanned image of six-day old zebrafish embryos. A wild-type embryo is shown at the top and two embryos containing a mutation in the Ornithine Decarboxylase nucleic acid sequence are shown at the bottom of the panel.

FIG. 73A is a scanned image of a four-day old zebrafish embryo, dorsal view. A wild-type embryo is shown.

FIG. 73B is a scanned image of a four-day old zebrafish embryo, dorsal view. An embryo containing a mutation in the Protein Phosphatase 1 Nuclear Targeting Subunit (PNUTS) nucleic acid sequence is shown at the bottom of the panel.

FIG. 74A is a scanned image of three-day old zebrafish embryos, lateral views. A wild-type embryo is shown at the left of the panel, and an embryo containing a mutation in the Mitochondrial Inner Membrane Translocating Protein (rTIM23) nucleic acid sequence is shown at the right of the panel. The mutant embryo displays smaller lighter eyes than the wild-type embryo. The mutant also displays pooling of blood around the heart, and some mutants have slower tail circulation.

FIG. 74B is a scanned image of three-day old zebrafish embryos, dorsal views. A wild-type embryo is shown at the top of the panel, and an embryo containing a mutation in the Mitochondrial Inner Membrane Translocating Protein (rTIM23) nucleic acid sequence is shown at the bottom of the panel.

FIG. 75A is a scanned image of three-day old zebrafish embryos, lateral views. A wild-type embryo is shown at the top of the panel, and embryos containing mutations in the 1447 nucleic acid sequence are shown below.

FIG. 75B is a scanned image of four-day old zebrafish embryos, ventral views. A wild-type embryo is shown at the top of the panel, and an embryo containing a mutation in the 1447 nucleic acid sequence is shown at the bottom of the panel.

FIG. 76A is a scanned image of three-day old zebrafish embryos, dorsal views. A wild-type embryo is shown at the top of the panel, and an embryo containing a mutation in the ARS2 nucleic acid sequence is shown at the bottom of the panel.

FIG. 76B is a scanned image of a five-day old zebrafish embryo, lateral view. This embryo contains a mutation in the ARS2 nucleic acid sequence

FIG. 77A is a scanned image of three-day old zebrafish embryos, lateral views. A wild-type embryo is shown at the top of the panel, and an embryo containing a mutation in the Sec61 Alpha nucleic acid sequence is shown at the bottom of the panel.

FIG. 77B is a scanned image of four-day old zebrafish embryos, lateral views. A wild-type embryo is shown at the top of the panel, and embryos containing a mutation in the Sec61 Alpha nucleic acid sequence are shown below it.

FIG. 78 is a scanned image of two-day old zebrafish embryos. A wild-type embryo is shown at the top and three embryos containing a mutation in the BAF53a nucleic acid sequence are shown at the bottom of the panel.

FIG. 79A is a scanned image of three-day old zebrafish embryos, lateral views. A wild-type embryo is shown at the top of the panel, and an embryo containing a mutation in the Histone Deacetylase nucleic acid sequence is shown at the bottom of the panel.

FIG. 79B is a scanned image of three-day old zebrafish embryos, dorsal views. A wild-type embryo is shown at the right of the panel, and an embryo containing a mutation in the Histone Deacetylase nucleic acid sequence is shown at the left of the panel.

FIG. 80A is a scanned image of a four-day old zebrafish embryo, lateral view. A wild-type embryo is shown.

FIG. 80B is a scanned image of a four-day old zebrafish embryo, lateral view. An embryo containing a mutation in the Fibroblast Isoform of the ADP/ATP Carrier Protein nucleic acid sequence is shown.

FIG. 81 is a scanned image showing a 48-hour old wild-type zebrafish embryo (Top) and a 48-hour old zebrafish embryo containing a mutation in a 459 nucleic acid sequence (Bottom).

FIG. 82 is a scanned image of a four-day old zebrafish embryo containing a mutation in a 459 nucleic acid sequence. The arrow points to a kidney cyst, which is also circled with a dashed line.

FIG. 83A is a scanned image of a transverse section through a wild-type zebrafish embryo. The arrow points to the kidney tubule and the notochord is labeled “nc.”

FIG. 83B is a scanned image of a transverse section through a zebrafish embryo containing a mutation in a 459 nucleic acid sequence. The arrow points to the kidney tubule which is swollen into a large cyst. The notochord is labeled “nc.”

FIG. 84 is a scanned image of a dorsal view of a 45-hour old wild-type zebrafish embryo. This embryo was stained with a polyclonal antibody raised against the C-terminal region of a 459 polypeptide. The dotted line represents the lateral boundary of one of the kidney tubules. The staining is localized to the apical surface of the epithelial cells lining the tubule.

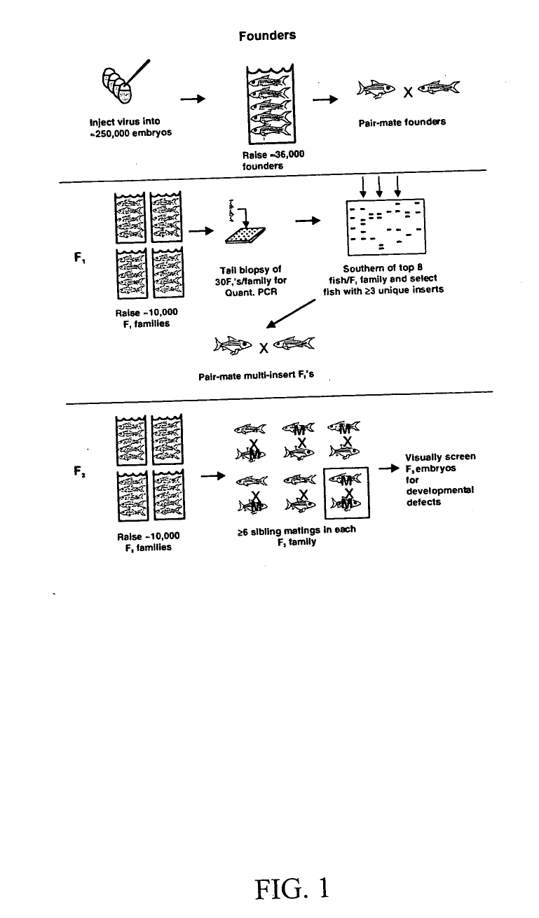

### DETAILED DESCRIPTION

We devised a strategy for the most efficient breeding and screening of proviral insertions so that we could perform a large-scale screen. By breaking this multi-step experiment into component projects and designing a protocol for each, we established a successful method for identifying vertebrate genes that are essential for normal development. Here we describe the screening methods and report on the first 80 developmental mutants obtained in our screen and the rapid cloning of the mutated genes.

The intent of this screen is to recover all embryonic mutations visible in a dissecting microscope at 1, 2, and 5 days post fertilization. As was found in large chemical mutagenesis screens in the zebrafish, mutants identified in this way include those with highly specific developmental defects involving one or a few organ systems and mutants that display one of several more general, and frequently recurring “syndromes.” In our experience, some mutants fall between these two groups, having specific aspects in combination with more widespread abnormalities. We refer to these phenotypes as “mixed.” Like almost all embryonic mutations ever isolated in the zebrafish by any method of screening, our mutants are recessive lethals. Most homozygous mutant embryos die between 3-10 days of age.

The general screening method used to identify the 80 genes described in this application involved the following steps. First, we injected high titer retrovirus into zebrafish embryos at the 1000-2000 cell stage. Approximately 36,000 of these injected fish (founders) were raised and pair-mated to generate 10,000 families of F1 fish. To identify fish with the most non-overlapping proviral inserts, 30 fish from each F1 family were analyzed by real-time quantitative PCR analysis of DNA extracted from tail fin clips. The tail fin clips from the eight fish containing the greatest number of proviral inserts, as assessed by real time quantitative PCR analysis, were further characterized using Southern blot hybridization. Fish with at least 3 unique proviral inserts were selected and pair-mated to generated 10,000 F2 families. The F2 fish were raised and siblings from these F2 families were crossed to generate F3 families, which were visually screened for developmental defects.

Since the viral vectors used in the experiments described herein generate a 4 bp duplication when they integrate, the insertion sites described herein are the approximate locations within the given nucleic acid sequences and the exact location may vary by as many as 4 nucleotides in either direction from that provided in the following descriptions. Described herein are the first 80 of the developmental mutants identified in the screen thus far.

The 904 Gene

Insertional mutations in the 904 nucleic acid sequence result in a severe disorganization of the brain and central nervous system (CNS) including, an overgrowth of neuronal tissue, a lack of definition of the brain compartments, increased vascularization, and brain hemorrhages. These phenotypes are already apparent two days into development and became more pronounced during day three, four, and five. 904 mutant embryos are still alive by day five as evidenced by the heart continuing to beat and the embryo responding to touch. In addition, a tail kink becomes evident by day four of development. The tail kink is indicative of the neural tube being disorganized.

We mapped the insertion site of the F5 virus in the 904 nucleic acid sequence to be approximately at nucleotide 1315 of SEQ ID NO:1. Analysis of the 904 amino acid sequence showed that it contains a zinc-binding domain which spans amino acids 80-125 of SEQ ID NO:2, and which may be important for protein degradation and cell cycle regulation. The 904 amino acid sequence also contains ankyrin repeats.

In addition, the zebrafish 904 amino acid sequence is 83% identical and 87% similar to a Drosophila melanogaster protein of unknown function (the CG 5841 gene product; GenBank Accession No. AAF49551.1) over a region encompassing amino acids 8 to 236 of SEQ ID NO:2. The 904 amino acid sequence is also 92% identical and 96% similar to the protein encoded by Homo sapiens clone IMAGE:3350926 (GenBank Accession No. BE255862) the over a region spanning 151 amino acids of SEQ ID NO:2. Furthermore, the 904 nucleic acid sequence (SEQ ID NO:1) is 79% identical over a region spanning 469 nucleotides of the human IMAGE:3350926 nucleotide sequence.

The POU2 Gene

We isolated zebrafish mutants containing a virus insertion in the POU2 gene (GenBank Accession No. D28548). This gene was previously described by Takeda et al. (Genes & Dev. 8:45-49, 1994). POU domain proteins are a large family of transcriptional regulatory proteins that may play roles in the regulation of gene expression in early development. The zebrafish POU2 protein is 37% identical and 47% similar to the human Brain-1 protein (GenBank Accession No. NP_{—}006227.1) over a stretch of amino acids encompassing amino acids 81 to 426 of SEQ ID NO:4. The nucleotide sequence encoding the human Brain-1 protein is 76% identical over 196 nucleotides to the zebrafish POU2 gene (SEQ ID NO:3; GenBank Accession No. D28548).

The POU2 mutants that we isolated display a lack of a mid-brain/hind-brain boundary and only have one otolith. This phenotype is visually detectable on day one of development, but may be observed earlier by in situ hybridization. In addition, the phenotype becomes more pronounced over time, but POU2 mutant embryos are still alive on day five of development.

The hair cells of the otolith can be visualized by staining for actin bundles. Accordingly one skilled in the art can readily determine the number of hair cells in a developing zebrafish embryo. POU2 mutant embryos may therefore be used in screens for test compounds that affect the number of hair cells in these embryos. A test compound that, when contacted with a POU2 embryo, results in an increase in the number of hair cell, is a candidate neuroprotective compound.

We mapped the insertion site of the virus in the POU2 gene and found the F5 virus insertion of allele hi349 to be approximately at nucleotide 1088, and the GT virus insertion of allele hi1940 to be at nucleotide 653, of SEQ ID NO:3.

The 40S Ribosomal Protein S18 Gene

We isolated zebrafish mutants containing a viral insertion in the 40S Ribosomal Protein S18 gene (GenBank Accession No. AF210641). We determined the GT virus insertion to be between nucleotides 220 and 221 of SEQ ID NO:5. The zebrafish gene product is 97% identical and 98% similar to the Mus musculus or Homo sapiens 40S Ribosomal Protein 18S (GenBank Accession Nos. NP_{—}035426.1 and P25232, respectively) over 152 amino acids of SEQ ID NO:6. The zebrafish gene is 82% identical over 410 nucleotides of SEQ ID NO:5 to the human 40S Ribosomal Protein S18.

Zebrafish mutant for the 40S Ribosomal Protein S18 have a head and eyes that are at least 50% smaller than those of identically aged wild-type zebrafish, a kinked tail, a reduced forebrain (generally only 50% the size of wild-type), and a bigger hind-brain. These defects may be observed on day two of development, but the embryos continue to be alive at day five of development.

The U2AF Gene

We isolated zebrafish mutants containing a viral insertion in the U2 Small Nuclear Rna Auxiliary Factor (U2AF) gene and found the insertion to be approximately between nucleotides 46 and 47 of SEQ ID NO:7 (GT virus for allele hi2505A and F5 virus for allele hi199). The zebrafish gene product is 92% identical and 94% similar to the human U2AF protein (GenBank Accession No. NP_{—}006749.1) in the region encompassing amino acids 1-249 of SEQ ID NO:8. In addition, the zebrafish U2AF gene is 82% identical to the Mus musculus homologue (GenBank Accession No. AK012334) over a region encompassing 537 nucleotides of SEQ ID NO:7. This zebrafish gene is also 82% identical to the Homo sapiens homologue (GenBank Accession No. XM_{—}009787.3) over a region encompassing 491 nucleotides of SEQ ID NO:7.

The zebrafish U2AF mutants display general brain necrosis by day two of development, with a particularly strong effect in the tectum.

The 954 Gene

We isolated zebrafish mutants containing a GT viral insertion approximately at nucleotide 432 or 506 of the 954 gene (SEQ ID NO:9). The coding region of the 954 gene is contained in the region spanning nucleotides 583 to 1252 of SEQ ID NO:9. The zebrafish 954 gene product is 93% identical and 96% similar to the human FLJ23591 protein (Accession No. NP_{—}079352.1).

In these mutants, cartilage does not stain with Alcian blue, but cartilage cells are visible in tissue sections. The zebrafish 954 gene is similar to Arabidopsis and Synechosystis dTDP-glucose 4-6-dehydratase.

The Nrp-1 Gene

We isolated zebrafish mutants containing a viral insertion in the Neurogenin Related Protein-1 (Nrp-1) gene (GenBank Accession No. AF036149). We determined the GT viral insertion to map approximately to nucleotide 1149 of SEQ ID NO:11. The Nrp-1 coding regions spans nucleotides 114 to 735 of SEQ ID NO:11 and the amino acid sequence is provided in SEQ ID NO:12.

Zebrafish mutant for the Nrp-1 gene have motility problems and are touch insensitive around the head, but not around the tail. By day five of development, zebrafish mutant for the Nrp-1 gene have a gaping jaw. In mice, a knockout mutation of this gene results in defects in cell fate determination of the neural crest.

The Cad-1 Gene

We isolated zebrafish mutants containing a viral insertion in the Caudal (Cad-1) gene (GenBank Accession No. X66958.1), a homeobox-containing transcription factor. We determined the GT virus insertion to be approximately between nucleotides 583 and 584 of SEQ ID NO:13 in both the hi2188A and 2092 alleles. The zebrafish Cad-1 gene product is 52% identical and 62% similar to the Homo sapiens homeobox protein CDX4 (GenBank Accession No. NP_{—}005184.1) over 258 amino acids of SEQ ID NO:14. In addition, the zebrafish Cad-1 gene is 80% identical to the Homo sapiens caudal type homeobox transcription factor 2 (CDX2; GenBank Accession No. XM_{—}039996.1) over 186 nucleotides of SEQ ID NO:13 and 83% identical to Homo sapiens CDX4 (GenBank Accession No. XM_{—}010453.1) over 102 nucleotides of SEQ ID NO:13.

On day one of development, these mutants have a shortened trunk and tail and no yolk sac extension. By day two of development, zebrafish mutant for Cad-1 are no longer motile and they are touch insensitive.

The V-ATPase Alpha Subunit Gene

We isolated zebrafish mutants containing an insertion of an F5 virus approximately at nucleotide 169 of the zebrafish V-ATPase Alpha Subunit gene (V-ATPase Alpha Subunit; SEQ ID NO:15). The zebrafish V-ATPase Alpha Subunit gene is 87% identical to Mus musculus clone 4930500C14 (GenBank Accession No. AK015654) over 185 nucleotides of SEQ ID NO:15, and 85% identical to the Homo sapiens LOC90423 gene (GenBank Accession No. XM_{—}031576.1) over 223 nucleotides of SEQ ID NO:15. In addition, the zebrafish V-ATPase Alpha Subunit gene product is 77% identical and 89% similar to the human homologue (GenBank Accession No. AAH04443) over a region spanning 226 amino acids of SEQ ID NO:16.

Mutants in this gene have reduced pigmentation in both the body and the eye.

The V-ATPase SFD Subunit Gene

We isolated zebrafish mutants containing an insertion of an F5 virus between approximately nucleotides 31 and 32 of the zebrafish VATPase SFD Subunit gene (SEQ ID NO:17). The coding region of the zebrafish V-ATPase SFD Subunit gene spans nucleotides 57 to 1445 of SEQ ID NO:17. The zebrafish V-ATPase SFD Subunit gene product is 89% identical and 94% similar to a Sus scrofa V H⁺-ATPase gene product (GenBank Accession No. AJ223757), and is 88% identical and 93% similar to the Homo sapiens MSTP042 protein (GenBank Accession No. AF 13222) over a stretch of 464 amino acids of SEQ ID NO:18. In addition, the zebrafish V-ATPase SFD Subunit gene is 81% identical to 537 nucleotides of the human LOC51606 mRNA over a region spanning 537 nucleotides of SEQ ID NO:17.

Mutants in this gene have reduced pigmentation in both the body and the eye by day three of development.

The V-ATPase 16 kDa Proteolytic Subunit Gene

We isolated zebrafish mutants containing an insertion of a virus approximately between nucleotides 242 and 243 of the zebrafish V-ATPase 16 kDa Proteolytic Subunit gene (SEQ ID NO:19). The coding region of the zebrafish V-ATPase 16 kDa Proteolytic Subunit gene spans nucleotides 170-631 of SEQ ID NO:19. We determined that the zebrafish V-ATPase 16 kDa Proteolytic Subunit gene is 86% identical to the Ovis aries homologue (GenBank Accession No. AF027705) over a region spanning 179 nucleotides, and 81% identical to the human homologue (GenBank Accession No. NM_{—}001694) over a region spanning 425 nucleotides, of SEQ ID NO:19. In addition, the zebrafish V-ATPase 16 kDa Proteolytic Subunit gene product is 91% identical and 94% similar to the corresponding human homologue (GenBank Accession No: P27449) over a region spanning 148 amino acids, and 91% identical and 95% similar to the mouse homologue (GenBank Accession No. NP_{—}033859.1) over a region spanning 149 amino acids, of SEQ ID NO:20.

Mutants in this gene have reduced pigmentation in both the body and the eye by 48 hours of development, as well as reduced touch sensitivity by 72 hours of development.

The 1463 Gene

We isolated zebrafish mutants containing an insertion of a GT virus approximately between nucleotides 389 and 390 in the zebrafish 1463 gene (SEQ ID NO:157). The coding region of the zebrafish 1463 gene spans nucleotides 266 to 1858 of SEQ ID NO:157. The zebrafish 1463 gene is 74% identical to the nucleic acid sequence encoding the Homo sapiens LimpII protein (GenBank Accession No. D12676) over a region spanning 175 bp of SEQ ID NO:157. In addition, the zebrafish 1463 gene product is 44% identical and 68% similar to the human Limp2 protein (GenBank Accession No. A56525) in a region encompassing amino acids 6-474 of SEQ ID NO:158.

These mutants have a defect in body pigmentation, but the eye is unaffected. Furthermore, 1463 mutants display brain dysmorphia including a swollen tectum, and a shorter hind-brain by day two of development and the defect becomes stronger as development progresses. In addition, the hind-brain is at least 10% shorter than wild-type. Zebrafish 1463 mutants also have less body pigment.

LimpII is part of a family of proteins which includes a transmembrane receptor for thrombospondin 1 (tsp1). In addition, LimpII has also been shown to bind Tsp1. Furthermore, Tsp1 is thought to be a naturally-occurring inhibitor of angiogenesis that limits vessel density in normal tissue and curtails tumor growth and progression (Jimenez et al., Nature Medicine 6:41-80, 2000; Tuszynski and Nicosia, Bioessays 18:71-76, 1996). Accordingly, the zebrafish 1463 gene may function in the development of vasculature in the brain and may be an important target for stroke therapy.

The VPSP18 Gene

We isolated zebrafish mutants containing an insertion of a GT virus approximately at nucleotide 2336 of the VPSP18 gene (SEQ ID NO:21), which encodes a protein that is 37% identical and 59% similar, over the C-terminal 592 amino acids of SEQ ID NO:22, to the Drosophila melanogaster Vacuolar Sorting Protein Deep Orange (GenBank Accession No. AE003421). In addition, the VPSP18 gene is 87% identical to the human Vacuolar Protein Sorting Protein 18 gene (GenBank Accession Nos. AF308802 and XP_{—}031478.1) over a stretch of 58 nucleotides of SEQ ID NO:21 and is 67% identical and 84% similar to this human protein over a region encompassing 604 amino acids of SEQ ID NO:22.

Mutants in the VPSP18 gene show a decrease in pigmentation, a lack of iridophores, and some necrosis in the tectum.

The Pescadillo Gene

We isolated zebrafish mutants containing a viral insertion in the Pescadillo gene, for example, approximately at nucleotide 20 of GenBank Accession No. U77627. This gene is required for the normal size of some, but not all, embryonic organs (Allende et al., Genes Dev. 10:3141-3155, 1996). In addition, the protein encoded by this gene contains a BRCF motif. Furthermore, Pescadillo was recently identified as a gene whose expression is elevated in p53 deficient tumor cell lines and it is now thought to play a role in cell cycle check points (Charpentier et al., Cancer Res. 60:5977-5983, 2000; Kinoshita et al., J. Biol. Chem. 276:6656-6665, 2001).

The HNF1-β/vHNF1 Gene

We isolated zebrafish mutants containing a viral insertion in the HNF1-β/vHNF1 gene. The hi548 allele is the result of an F5 virus insertion approximately at nucleotide 1682/1683, the hi1843 allele is the result of a GT virus insertion at nucleotide 745, and the hi2169 allele is the result of a GT virus insertion at nucleotide 361, of SEQ ID NO:23. The coding region of the zebrafish HNF1-β/vHNF1 gene is contained within nucleotides 143 to 1819 of SEQ ID NO:23. This gene is 84% identical to the Rattus norvegicus Hepatic Transcription Factor 1 gene (GenBank Accession No. NM_{—}012669.1) over 227 nucleotides of SEQ ID NO:23. This zebrafish gene, SEQ ID NO:23, also is identical to various stretches of the Homo sapiens Hepatic Transcription Factor 1 nucleotide sequence (GenBank Accession No. XM_{—}012120.3), 77% identical over 381 nucleotides, 78% over 375 nucleotides nucleotides, 80% identical over 270 nucleotides, 81% identical over 149 nucleotides, and 74% identical over 95 nucleotides. In addition, the zebrafish HNF1-β/vHNF1 protein (SEQ ID NO:24) is 80% identical and 87% similar to the human homologue (GenBank Accession Nos. NP_{—}000449.1, XP_{—}008554.1, S34412, X58840, and U90287).

Mutants in the HNF1-β vHNF1 gene display a cystic kidney and an abnormal pancreas. Zebrafish containing the hi2169 allele also have defects in the patterning of the hind-brain, resulting in defective ear (otolith) structures. In addition, mutations in the human HNF1-β/vHNF1 gene has also been found to cause of a genetic form of human diabetes, MODY V (maturity onset diabetes of the young), in which patients have kidney defects in addition to diabetes (Iwasaki et al., Diabetes Care 21:2144-2148, 1998; Horikawa et al., Nat. Genet. 17:384-385, 1997).

The 60S Ribosomal Protein L35 Gene

We isolated zebrafish mutants containing a viral insertion approximately between nucleotides 30 and 31 of the 60S Ribosomal Protein L35 nucleic acid sequence (SEQ ID NO:25). The coding region of the zebrafish 60S Ribosomal Protein L35 spans nucleotides 29-397 of SEQ ID NO:25 and this gene is 82% identical to the human homologue (GenBank Accession No. BC000348.1) over a region spanning 319 nucleotides of SEQ ID NO:25. In addition, the zebrafish 60S Ribosomal Protein L35 is 92% identical and 95% similar to the human protein (GenBank Accession No. NP_{—}009140.1) over a region spanning 123 amino acids of SEQ ID NO:26.

The zebrafish 60S Ribosomal Protein L35 mutants display an inflated ventricle, have a head and eyes that are at least 50% smaller than those of identically aged wild-type zebrafish by 48 hours of development, and have blurred somite boundaries.

The 60S Ribosomal Protein L44 Gene

We isolated zebrafish mutants containing a viral insertion in the 60S Ribosomal Protein L44 gene (described in Amsterdam et al. (Genes & Dev. 13:2713-2724, 1999)). We determined the viral insertion to be approximately between nucleotides 195 and 196 of the sequence of SEQ ID NO:27. Late in day one of development, these mutants have an enlarged brain ventricle and the yolk disappears from the yolk sac extension. 60S Ribosomal Protein L44 mutant embryos die by day four or five of development. The zebrafish 60S Ribosomal Protein L44 gene is 85% identical to the Mus musculus homologue (GenBank Accession No. NM_{—}019865.1) over a region spanning 324 nucleotides of SEQ ID NO:27. In addition, the zebrafish 60S Ribosomal Protein L44 gene product is 98% identical and 98% similar to the Mus musculus 60S Ribosomal Protein L44 gene product (GenBank Accession No. NP_{—}063918.1) over the region encompassing amino acids 1-106 of SEQ ID NO:28.

The CopZ1 Gene

We isolated zebrafish mutants containing an insertion of an F5 virus approximately between nucleotides 90 and 91 of the CopZ1 gene (SEQ ID NO:29; GenBank Accession No. AB040044). The coding region of this gene spans nucleotides 73 to 606 of SEQ ID NO:29 and the amino acid sequence of the CopZ1 gene product is provided in SEQ ID NO:30.

CopZ1 mutant zebrafish display a degeneration of the eye, especially in the retinal, pigmented epithelia. However, the neuronal layers of the retina also begin to degenerate starting on day four of development.

The 215 Gene

We isolated zebrafish mutants containing an insertion of an F5 virus approximately between nucleotides 294 and 295 of the 215 gene (SEQ ID NO:31). The coding region of the zebrafish 215 gene spans nucleotides 47-622 of SEQ ID NO:31 and the 215 gene product, which is similar to ATP-dependent RNA helicases, contains a DEAD-Box helicase domain between amino acids 24 and 228, as well as a Helicase C domain between amino acids 292 and 363. Furthermore, the zebrafish 215 gene is 81% identical to the human gene encoding the KIAA1595 protein (GenBank Accession Nos. AB046815 and BAB13421.1) over a region spanning 188 nucleotides of SEQ ID NO:31 and the gene product is 78% identical and 88% similar to the gene product of this human gene over a region spanning 393 amino acids of SEQ ID NO:32. The zebrafish 215 gene product is also 77% identical and 86% similar to the Mus musculus AK012782 gene product (GenBank Accession No. BAB28466.1) over a region spanning 529 amino acids of SEQ ID NO:32.

By day three of development, these mutants have eyes that are at least 75% smaller than those of three day-old wild-type zebrafish, a jaw that is at least 75% reduced when compared to that of a three day-old wild type zebrafish, and display general underdevelopment. In addition, using Alcian blue staining, we observed a bent ceratohyal.

The 307 Gene

We isolated zebrafish mutants containing an insertion of an F5 virus approximately at nucleotide 176 of the 307 gene (SEQ ID NO:33). The coding region of the 307 gene may begin either at nucleotide 333 or 339 of SEQ ID NO:33. The zebrafish 307 gene is 94% identical over a region spanning 34 nucleic acids of SEQ ID NO:33, and the 307 gene product is 54% identical and 68% similar over a stretch of 199 amino acids of SEQ ID NO:34, to human beta 1,3 glucuronyl transferase (GenBank Accession No. AB009598) and is required for the formation of cartilage and/or jaw structures. In addition, zebrafish containing a mutation in the 307 gene have a mandibular arch that does not extend anteriorly and have slightly misshapen branchial arches 3-7.

The 572 Gene

We isolated zebrafish mutants containing an insertion of an F5 virus approximately at nucleotide 277 of the 572 gene. The coding region of the 572 gene spans nucleotides 48-701 of SEQ ID NO:35. The 572 gene product is 37% identical to the human FLJ20508 gene product (GenBank Accession No. NP_{—}060320.1) over a stretch of 196 amino acids of SEQ ID NO:36. By day four of development, mutants in the 572 gene have shorter jaw and branchial arches which are fragmented and/or hard to see.

The 1116A Gene

We isolated zebrafish mutants containing an insertion of a GT virus approximately at nucleotide 135 of the 1116A gene (SEQ ID NO:37). The coding region of the 1116A gene spans nucleotides 33 to 578 of SEQ ID NO:37. The 1116A gene product is 42% identical to the human NIH_MGC_{—}93 cDNA clone (GenBank Accession No. BG287661) over a stretch of 191 amino acids of SEQ ID NO:38. By day three of development, we observed a failure of the jaw to develop in 1116A mutants, based on Alcian blue staining.

The 1548 Gene

We isolated zebrafish mutants containing an insertion of a GT virus approximately at nucleotide 85 of the 1548 gene (SEQ ID NO:39). The coding region of the 1548 gene spans nucleotides 99 to 2990 of SEQ ID NO:39. The zebrafish 1548 gene is 78% identical to the human gene encoding the DKFZP434B168 protein (GenBank Accession No. NM_{—}015434.1) over a stretch of 503 nucleotides of SEQ ID NO:39, and the nucleic acid sequence encoding the human protein (GenBank Accession No. NP_{—}056249.1) is 76% identical and 87% similar to the zebrafish 1548 gene over 963 nucleotides of SEQ ID NO:40. Zebrafish containing a 1548 mutation have eyes that are slightly smaller than those of identically aged wild-type zebrafish, an abnormal head shape, and edema around the eyes and heart. In addition, these mutants appear to have thicker pectoral fins and jaws. Furthermore, by Alcian blue staining, these mutants have an added structure attached to the parachordal in the neurocranium. While these phenotypes are visible by day three of development, they are more apparent by day five.

The Casein Kinase 1 α Gene

We isolated zebrafish mutants containing an insertion of a GT virus approximately between nucleotides 730 and 731 of the Casein Kinase 1 a gene (SEQ ID NO:41). The coding region of the zebrafish Casein Kinase 1 α gene spans nucleotides 505 to 1479 of SEQ ID NO:41. The zebrafish Casein Kinase 1 α gene is 84% identical to the Gallus gallus homologue (GenBank Accession No. AF042862) over a region spanning 976 nucleotides, and is 82% identical to the Homo sapiens Casein Kinase 1 α and clone PP2685 (GenBank Accession Nos. XM_{—}046995.1 and AF218004, respectively) over a region spanning 946 nucleotides, of SEQ ID NO:41. In addition, the zebrafish Casein Kinase 1 α gene product is 99% identical to various vertebrate homologues including the Gallus gallus (GenBank Accession Nos. AF042862 Y08817, and U80822), Rattus norvegicus (GenBank Accession No. U77582), and Homo sapiens (Genbank Accession Nos. XP_{—}046994.1, XP_{—}011309.3, and XP_{—}046996.1), over a region spanning 324 to 325 amino acids of SEQ ID NO:42.

The mutant phenotype indicates that this gene is required for the formation of cartilage and/or jaw structures. By day three of development, zebrafish mutant for the Casein Kinase 1 α gene have retarded development of the pectoral fins and some of these fins are misshapen. In addition, Alcian blue staining shows that the cartilage of the fins, branchial arches, and jaw is wrinkled.

The Nodal-Related (Squint) Gene

We isolated zebrafish mutants containing a viral insertion in the Nodal-Related (Squint) gene (Feldman et al., Nature 395:181-185, 1998; GenBank Accession No. AF056327). We determined that our Nodal-Related (Squint) mutant has a GT virus inserted approximately at nucleotide 654 of SEQ ID NO:43 (GenBank Accession No. AF002218). This location is equivalent to nucleotide 526 of GenBank Accession No. AF056327. The coding region of the zebrafish Nodal-Related (Squint) gene spans nucleotides 177-1355 of SEQ ID NO:43. The Squint gene is 43% identical and 61% similar to the Xenopus laevis Xnr5 gene (GenBank Accession No. BAB18971.1) over a region spanning 355 amino acids, 42% identical and 60% similar to the Xenopus laevis Xnr-2 gene (Genbank Accession No. AAA97393.1) over a region spanning 381 amino acids, 41% identical and 61% similar to the Xenopus laevis Xnr-6 gene (GenBank Accession No. BAB18972.1) over a region spanning 348 amino acids, 41% identical and 60% similar to the Xenopus laevis Xnr-1 gene (GenBank Accession No. AAA97392.1) over a region spanning 367 amino acids, 35% identical and 48% similar to the Homo sapiens Nodal-Related Protein (GenBank Accession No. BAB62524.1) over a region spanning 346 amino acids, and 33% identical and 48% similar to the Mus musculus Nodal-Related or Squint Protein (GenBank Accession No. NP_{—}038639.1) over a region spanning 344 amino acids, of SEQ ID NO:44.

The Smoothened Gene

We isolated zebrafish mutants containing a viral insertion in the Smoothened gene (described in Chen et al. (Development 128:2385-2396, 2001); GenBank Accession No. AY029808.1). In the Smoothened mutant zebrafish, we determined that, in the hi 229 allele, an F5 virus inserted approximately at nucleotide 271, and that, in the hi 1640 allele, a GT virus inserted approximately at nucleotide 600, of SEQ ID NO:45. The coding region of the Smoothened gene spans nucleotides 383-2815 of SEQ ID NO:45 and the amino acid sequence of the Smoothened gene product is provided in SEQ ID NO:46.

Mutations in this gene affect the central nervous system, resulting in a reduction in the number of neurons. Fewer primary motoneurons are present and their axons do not extend correctly. In addition, no secondary motoneurons can be observed in smoothened mutants. The forebrain and midbrain commissures do not form and the optic nerves fail to reach and cross the midline. Further phenotypes observed in smoothened mutants are abnormalities in body shape/axial structures (the body is curved ventrally, the floorplate is reduced, the horizontal myoseptum is missing, the somites are U-shaped, not V-shaped, and mild cyclopia is observed), in cartilaginous structures (the jaw, branchial arches, and pectoral fins are absent), and in muscles (a lack of adaxial muscle tissue, slow muscle fibers, and muscle pioneer cells is observed).

The 429 Gene

We isolated zebrafish mutants containing an insertion of an F5 virus approximately between nucleotides 182 and 183 of the 429 gene (SEQ ID NO:47). The coding region of the 429 gene spans nucleotides 43 to 2301 of SEQ ID NO:47. These mutants have a very small liver with no visible circulation, no gall bladder, no pancreas, and the gut is underdeveloped. These phenotypes begin to appear by day three of development and are easily discernible by day five of development. The zebrafish 429 gene product is 53% identical and 70% similar to a human protein (GenBank Accession No. NP_{—}055203.1) over a region spanning 765 amino acids of SEQ ID NO:48 and is 53% identical and 68% similar to the Mus musculus bM282D4.5 protein (GenBank Accession No. CAC42185.1) protein over a region spanning 771 amino acids of SEQ ID NO:48.

The 428 Gene

We isolated zebrafish mutants containing an insertion of a GT virus approximately at nucleotide 187 of the 428 gene (SEQ ID NO:49). The coding region of the 428 gene spans nucleotides 44-629 of SEQ ID NO:49. This gene is 85% identical to the Mus musculus 2810405O22Rik sequence (GenBank Accession No. NM_{—}026042.1) over a region spanning 137 nucleotides of SEQ ID NO:49, and is 89% identical to the Homo sapiens DKFZp434H247 protein (GenBank Accession Nos. XM_{—}046121.1 and AL137304.1) over a region spanning 68 nucleotides of SEQ ID NO:49. In addition, the 428 gene product is 62% identical and 71% similar to the Homo sapiens DKFZp434H247 protein (GenBank Accession Nos. CAB70687.1 and BAA91147.1) over a region spanning 170 amino acids of SEQ ID NO:50.

Zebrafish mutant for the 428 gene have more muscle septa and striations than identically aged wild-type zebrafish. In addition, by day five of development these zebrafish have necrosis of the brain.

The Spinster Gene

We isolated zebrafish mutants containing a viral insertion in the spinster gene. These mutants are the result of an SGF virus inserted several kilobases, e.g., 2 to 5 kb, upstream of the coding region, which spans nucleotides 209-1729 of the Spinster gene (SEQ ID NO:51). The zebrafish Spinster gene is 83% identical to the human clone IMAGE:3627317 sequence (GenBank Accession No. BC006156) over a region spanning 81 nucleotides of SEQ ID NO:51. In addition, the zebrafish spinster gene product is 64% identical and 75% similar to the Homo sapiens (GenBank Accession Nos. NP_{—}114427.1, AAG43830.1, and AAH08325.1) or Mus musculus (GenBank Accession Nos. NP_{—}076201.1 and AAG43831.1) Spinster-Like Proteins over a region spanning 527 or 528 amino acids, respectively, of SEQ ID NO:52.

Zebrafish mutant for this gene show a degeneration of the yolk by day two of development, resulting in a gradual death of the embryo.

The Glypican-6 or Knypek Gene

We isolated zebrafish mutants containing an insertion of a GT virus approximately at nucleotide 1054 (hi 1688 allele) or nucleotide 133 (hi 1934 allele) of the Glypican-6 or Knypek gene (SEQ ID NO:53). The coding region of the zebrafish Glypican-6 or Knypek gene spans nucleotides 302-1972 of SEQ ID NO:53. The zebrafish Glypican-6 or Knypek gene (GenBank Accession No. AF354754) is 87% identical to the Homo sapiens Glypican 4 gene (GenBank Accession No. XM_{—}010339.3) over a region spanning 86 nucleotides, and is 82% identical to the Homo sapiens Glypican 6 gene (GenBank Accession Nos. XM_{—}015995.2 and AF105267.1) over a region spanning 133 nucleotides, of SEQ ID NO:53. In addition, the zebrafish Glypican-6 or Knypek gene product is 56% identical and 75% similar to the Mus musculus Glypican 6 Protein (GenBank Accession No. AF105268_{—}1) over a region spanning 541 amino acids, and is 55% identical and 74% similar to the Homo sapiens Glypican 6 Protein (GenBank Acession Nos. AF111178_{—}1 and AF105267_{—}1) over a region spanning 542 amino acids, of SEQ ID NO:54. Furthermore, the zebrafish Glypican-6 or Knypek gene product is 58% identical and 72% similar to the Mus musculus Glypican 4 Protein (GenBank Accession No. NP_{—}032176.1) over a region spanning 533 amino acids, and is 56% identical and 70% similar to the Homo sapiens Glypican 4 Protein over a region spanning 550 amino acids, of SEQ ID NO:54.

By day one of development, zebrafish containing a mutation in the Glypican-6 or Knypek gene have a shortened tail and U-shaped somites.

The Ribonucleotide Reductase Protein R1 Class 1 Gene

We isolated zebrafish mutants (hi 318 and hi 2769A) containing a viral insertion in the Ribonucleotide Reductase Protein R1 Class 1 gene (GenBank Accession No. U57964). The hi 318 allele has an insertion of an F5 virus, and the hi 2769A allele has an insertion of a GT virus, approximately between nucleotides 147 and 148 of SEQ ID NO:55. The coding region of the zebrafish Ribonucleotide Reductase Protein R1 Class 1 gene spans nucleotides 131 to 2512 of SEQ ID NO:55.

Zebrafish having a mutation in this gene have a bent, convex body shape. In addition, these mutants display transient brain and eye necrosis between 24 and 48 hours of development.

The Kinesin-Related Motor Protein EG5 Gene

We isolated zebrafish mutants (alleles hi 486 and hi 3112A) containing a viral insertion in the Kinesin-Related Motor Protein EG5 gene. The hi 486 allele contains an insertion of an F5 virus, ad the hi 3112A allele contains an insertion of a GT virus, approximately between nucleotides 50 and 51 of SEQ ID NO:57. This gene is 80% identical to the Xenopus laevis gene encoding a kinesin-like protein (GenBank Accession No. X71864.1) over a region spanning 538 nucleotides of SEQ ID NO:57. The zebrafish Kinesin-Related Motor Protein EG5 gene is also 80% identical to the Mus musculus Kinesin-Related Mitotic Motor Protein gene (GenBank Accession No. AJ223293.1) over 320 nucleotides, and is 80% identical to the Homo sapiens Kinesin-Like 1 gene (GenBank Accession No. XM_{—}005889.4) over 310 nucleotides, of SEQ ID NO:57. Furthermore, the zebrafish Kinesin-Related Motor Protein EG5 gene product is 55% identical and 71% similar to the Xenopus laevis homologue (GenBank Accession Nos. P28025, A40264, and CAA37950.1) over a region spanning 948 amino acids of SEQ ID NO:58. This protein is also 50% identical and 67% similar to the Homo sapiens Kinesis-Like Spindle Protein HSKP (GenBank Accession Nos. NP_{—}004514.2, XP_{—}051151.1, XP_{—}051152.1, XP_{—}005889.3, G02157, and AAA86132.1), and the Homo sapiens Kinesin-Related Protein EG5 (Genbank Accession Nos. P52732 and CAA59449.1), over a region spanning 948 amino acids of SEQ ID NO:58.

While the mutant phenotype is not 100% penetrant, zebrafish mutant for the Kinesin-Related Motor Protein EG5 gene generally have bent bodies. In addition, these mutant embryos display elevated levels of apoptotic cells on their surface by 48 hours of development.

The 459 Gene

We isolated zebrafish mutants containing an insertion of an F5 virus approximately at nucleotide 210 of the 459 nucleic acid sequence (SEQ ID NO:59). The zebrafish 459 nucleic acid sequence is 74% identical to the Xenopus laevis clone IMAGE:3744559 (GenBank Accession No. BG016943.1) over a region spanning 415 nucleotides of SEQ ID NO:59. In addition, this zebrafish nucleic acid sequence is 73% identical to the Mus musculus clone 4933412L17 (GenBank Accession No. AK016794.1) over a region spanning 584 nucleotides of SEQ ID NO:59. In addition, the polypeptide encoded by the 459 nucleic acid sequence is 72% identical and 90% similar to the polypeptide encoded that the Xenopus laevis IMAGE:3744559 clone (GenBank Accession No. BG016943.1) over a region spanning 233 amino acids of SEQ ID NO:60 and is 80% identical and 92% similar to the Mus musculus IMAGE 1245647 clone (GenBank Accession No. AA797813.1) polypeptide over a region spanning 200 amino acids of SEQ ID NO:60.

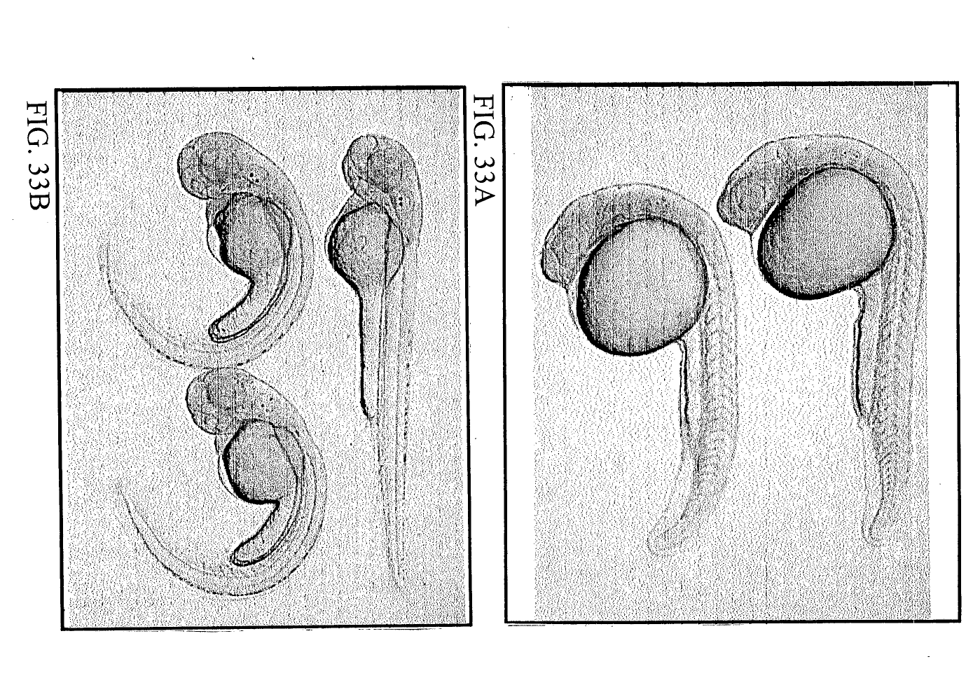

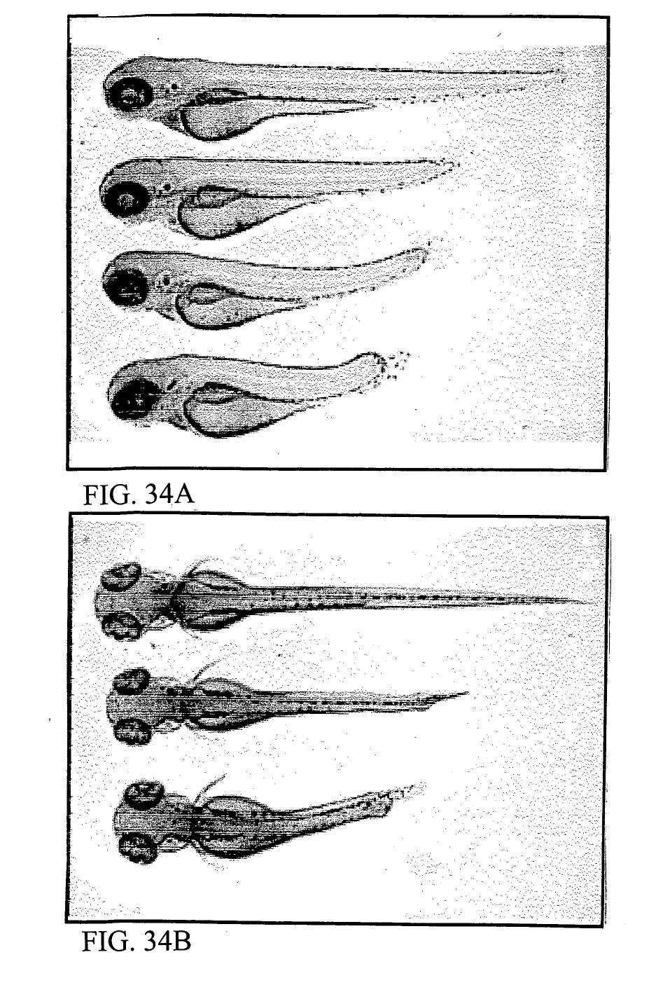

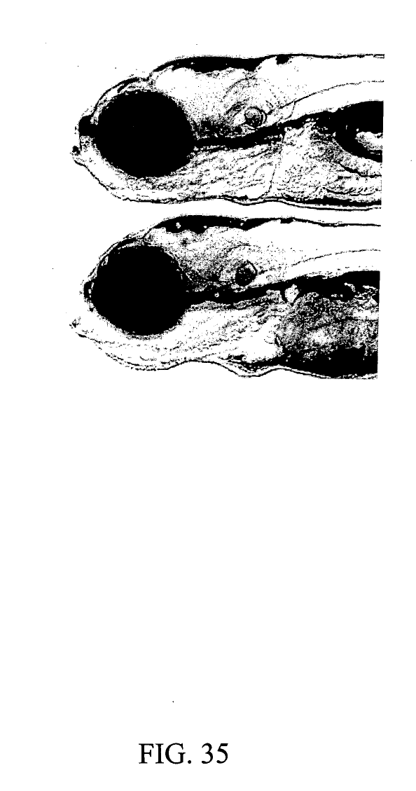

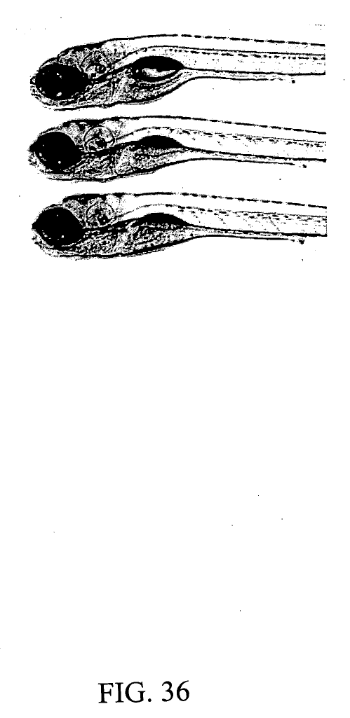

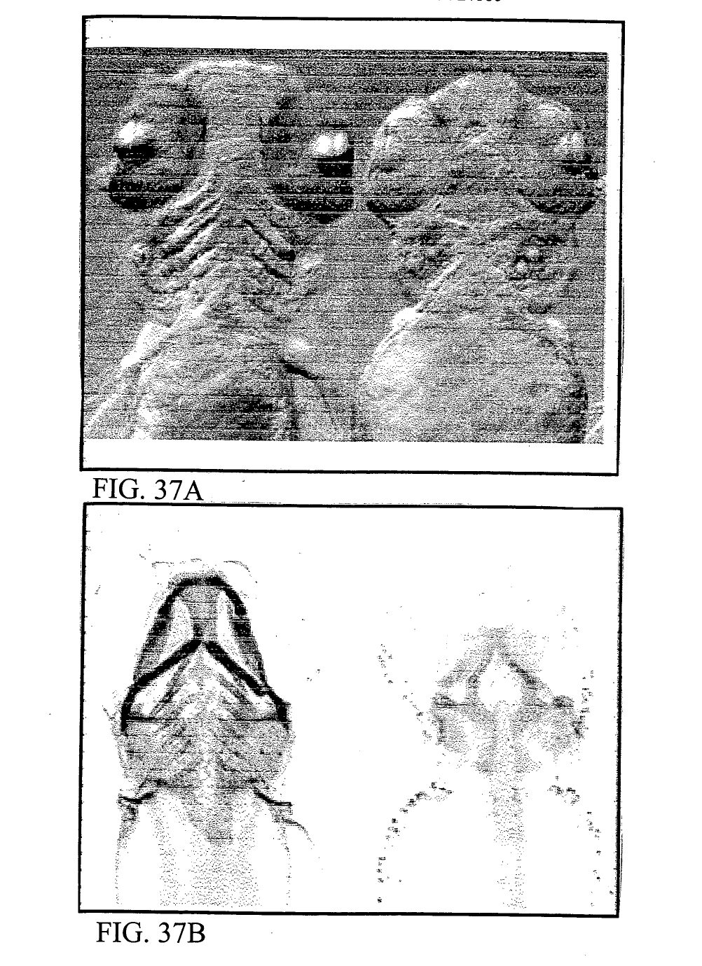

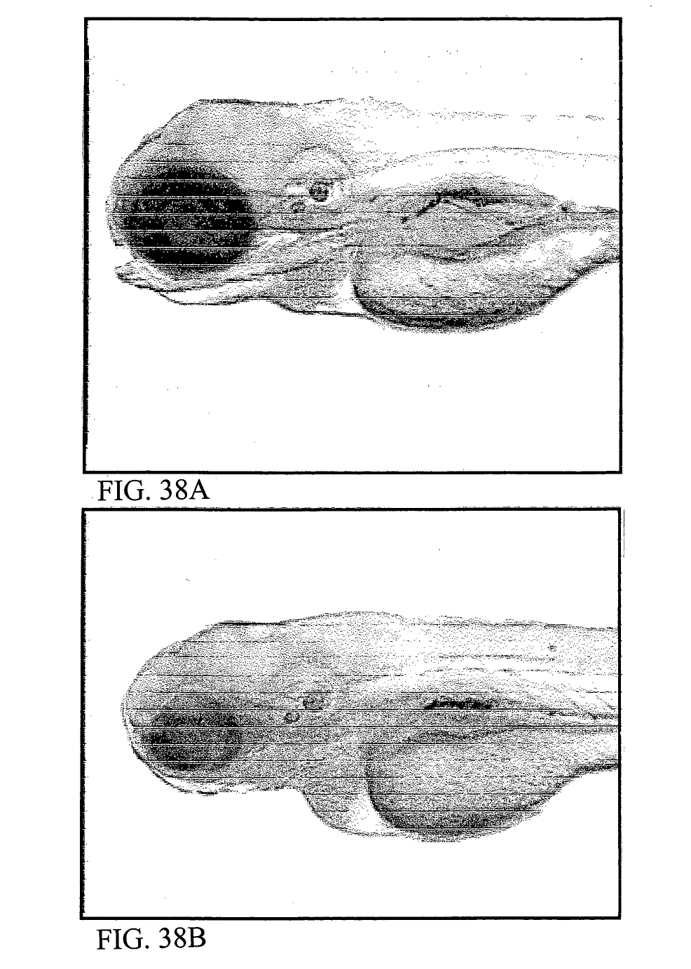

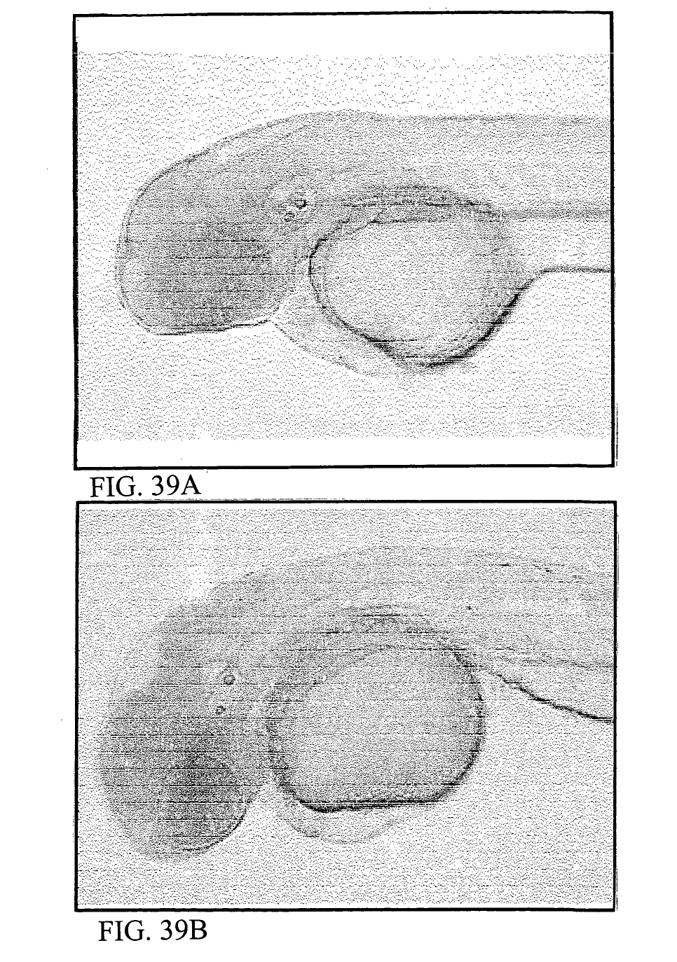

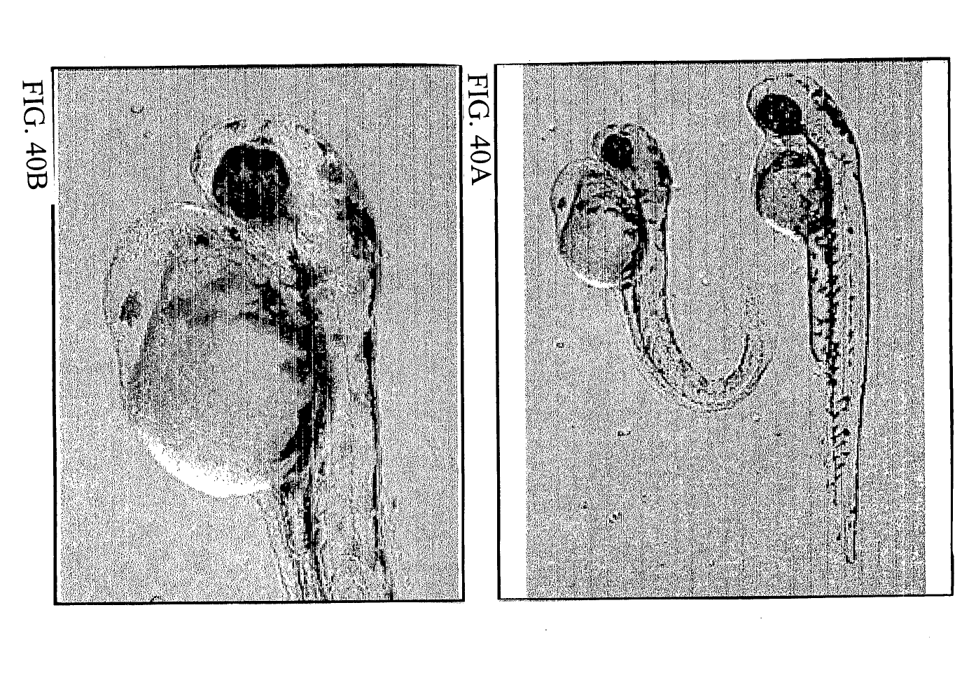

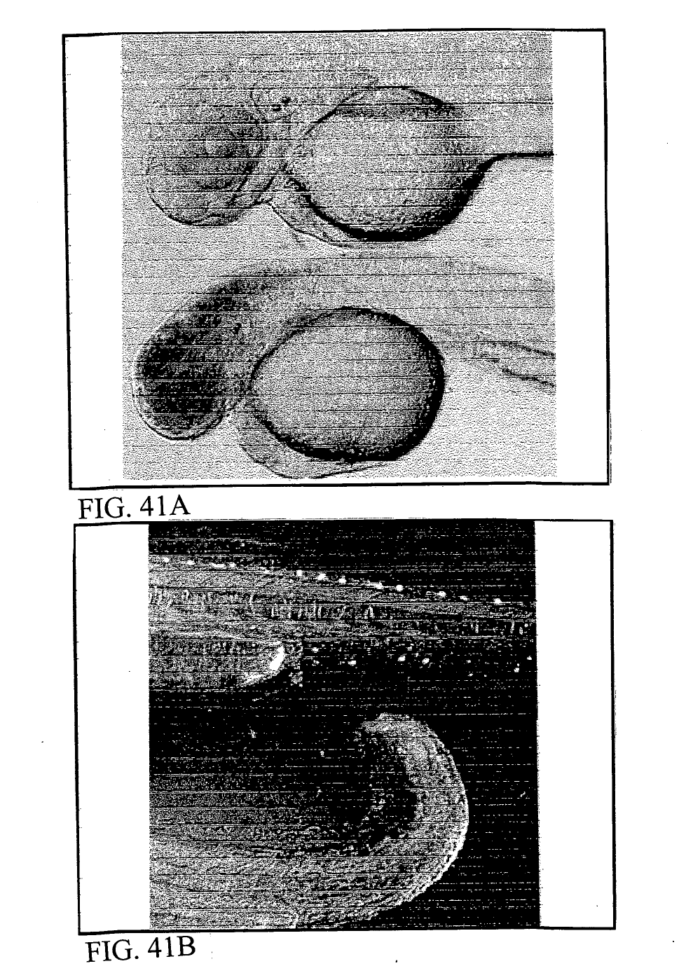

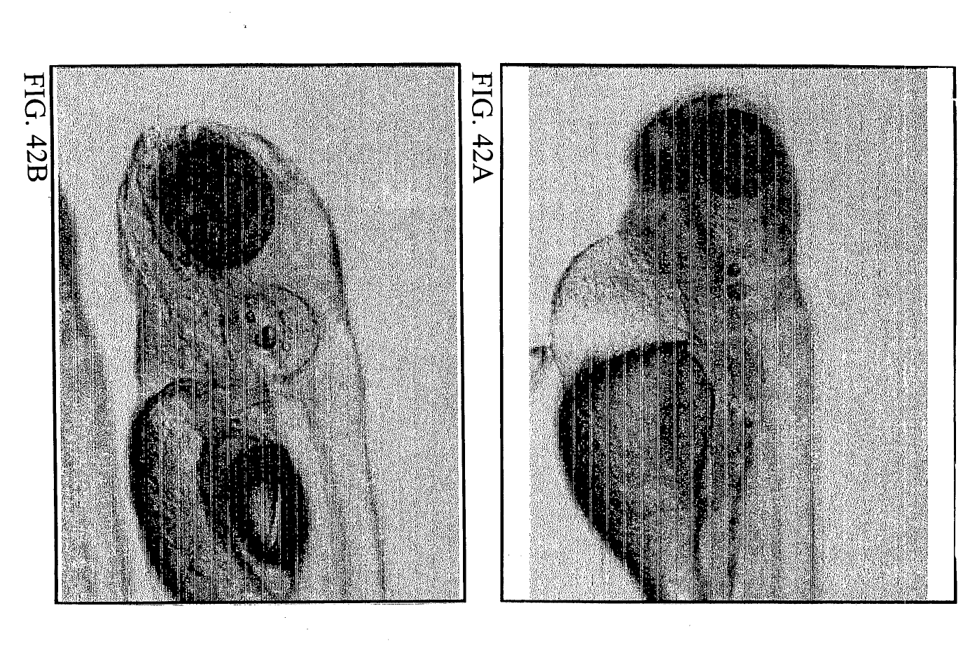

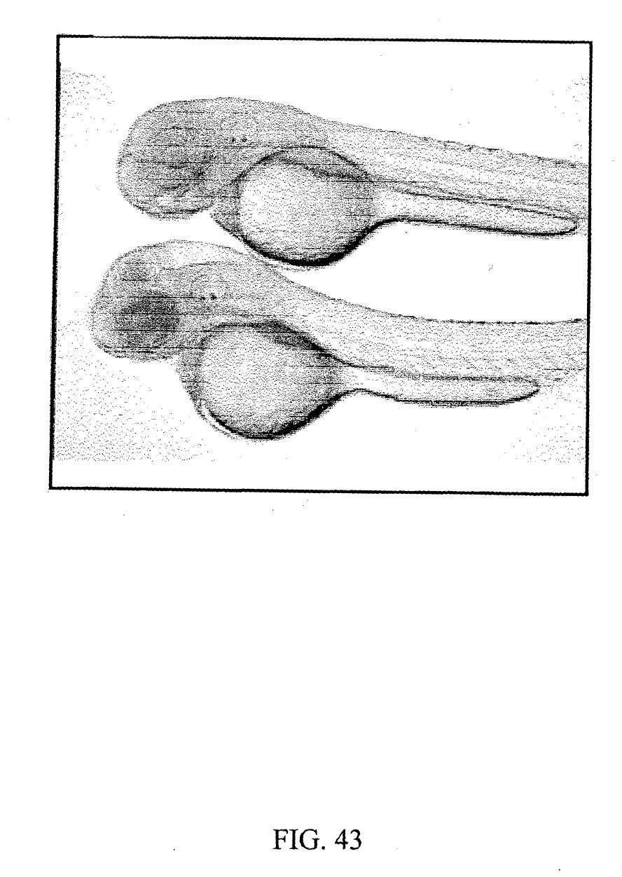

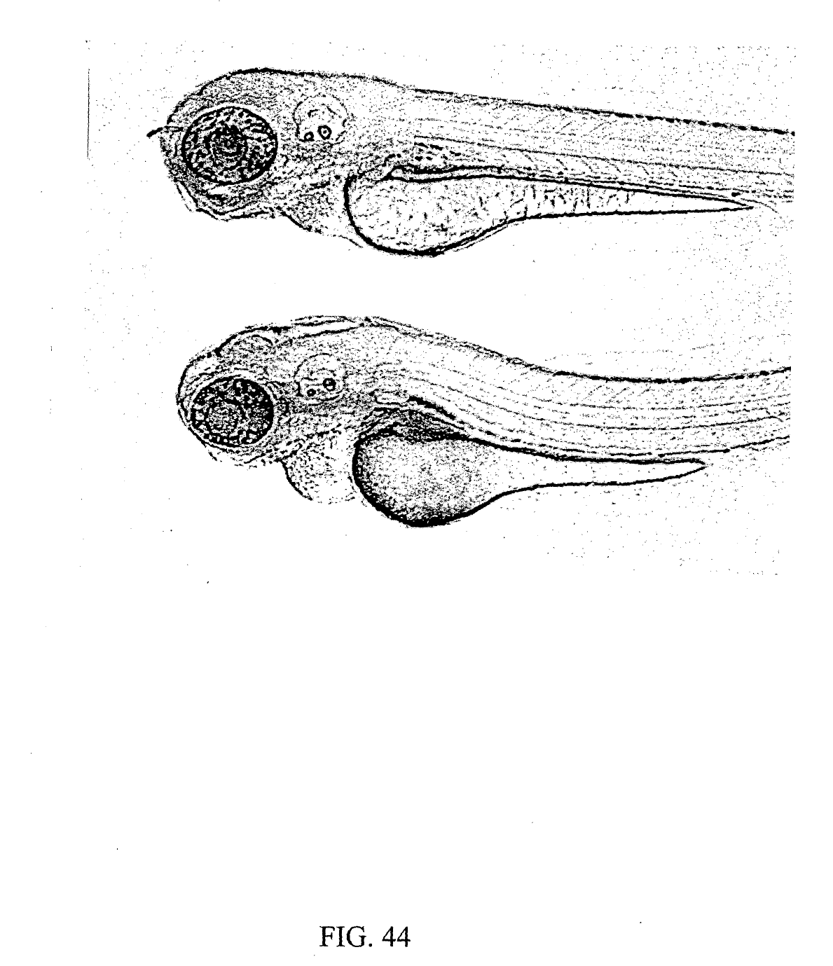

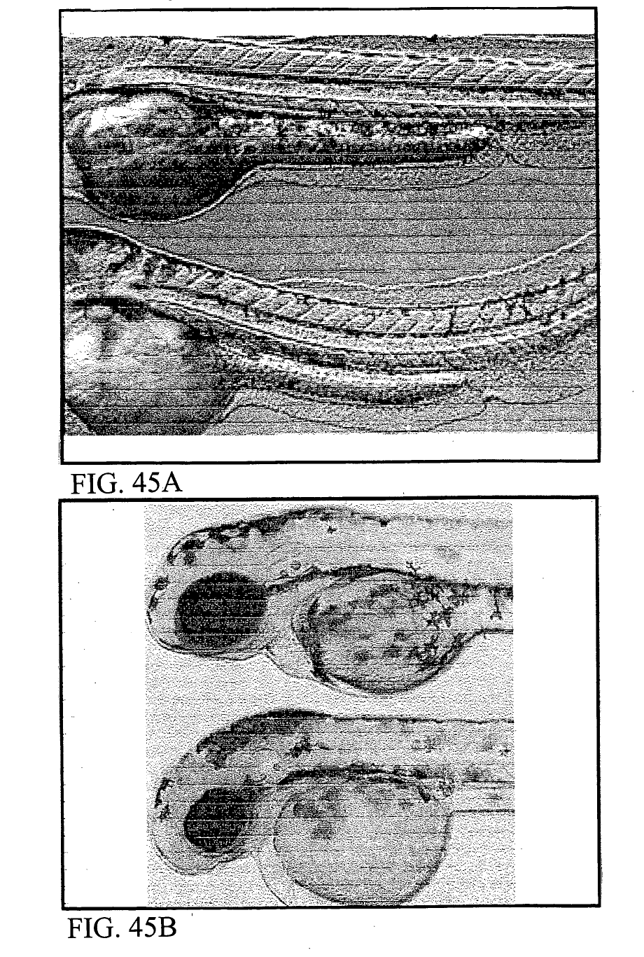

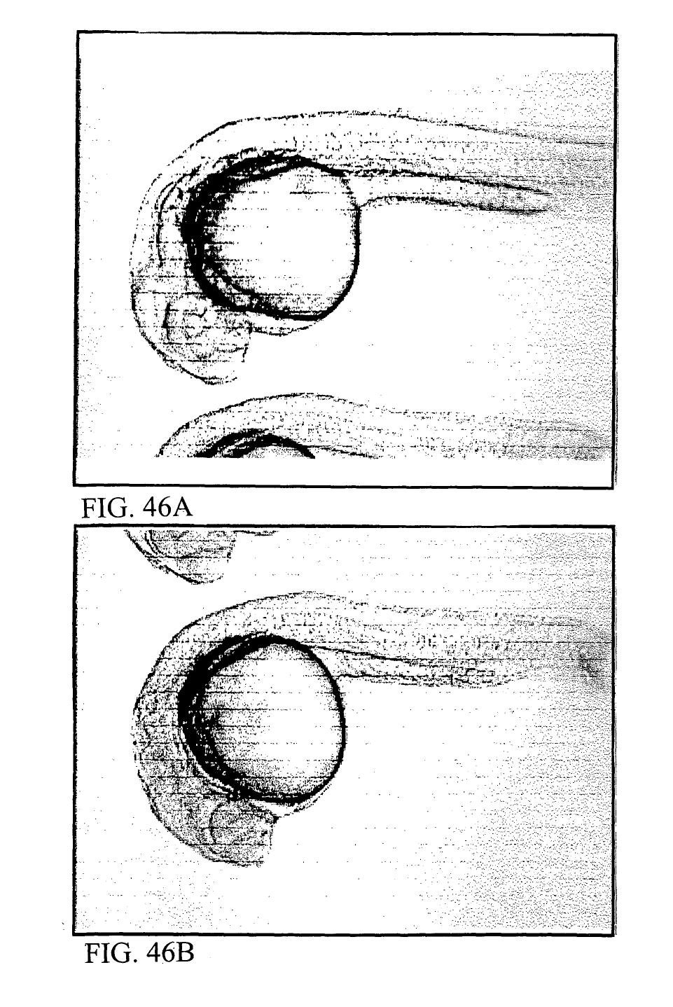

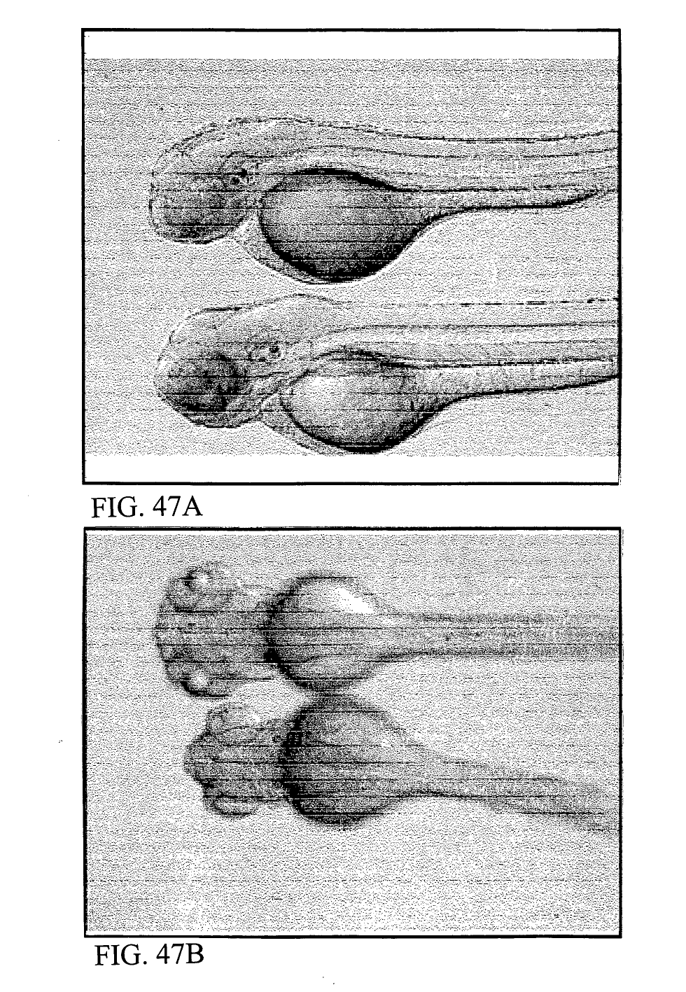

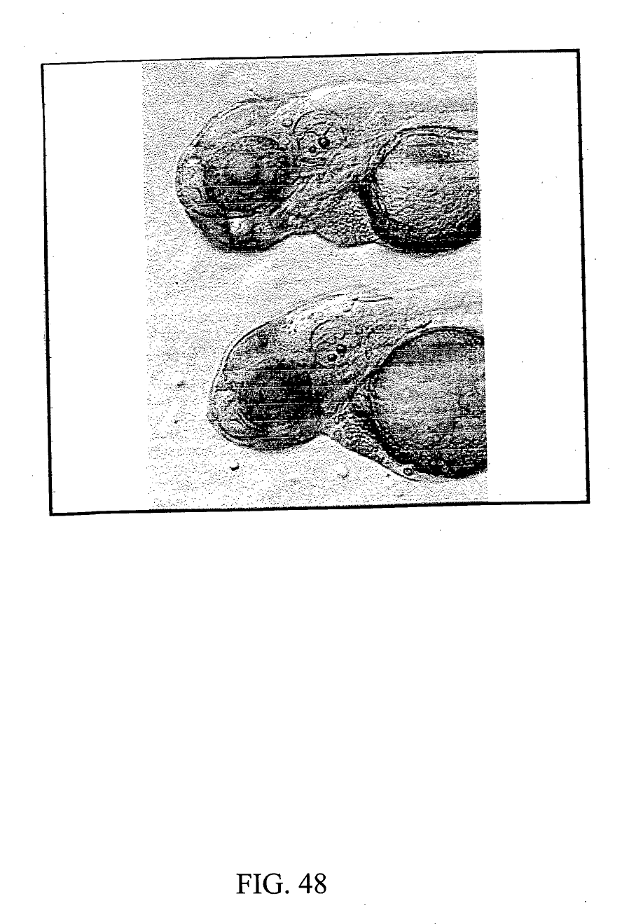

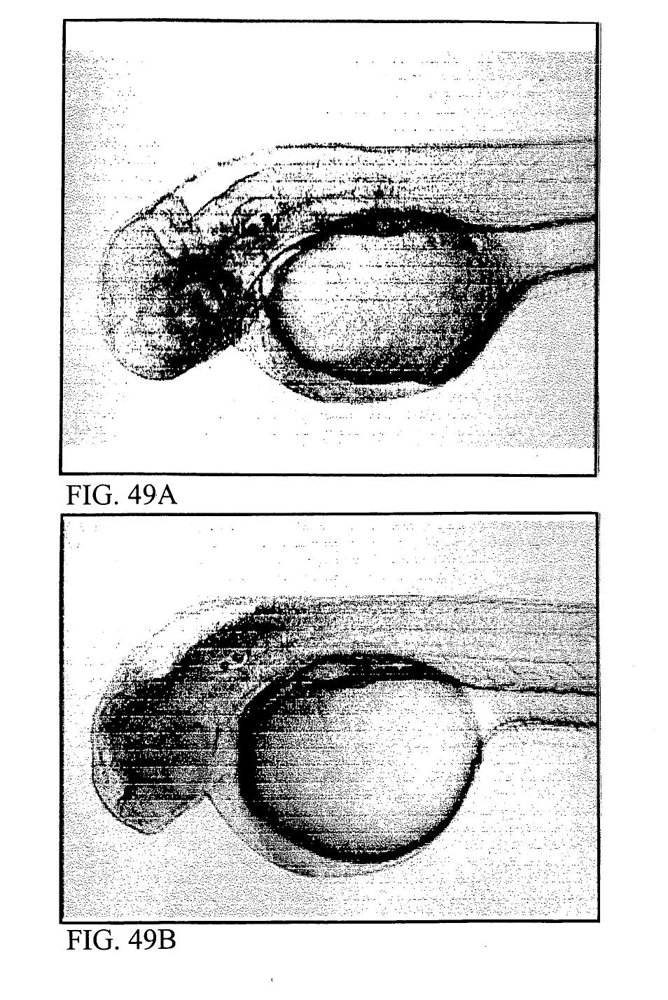

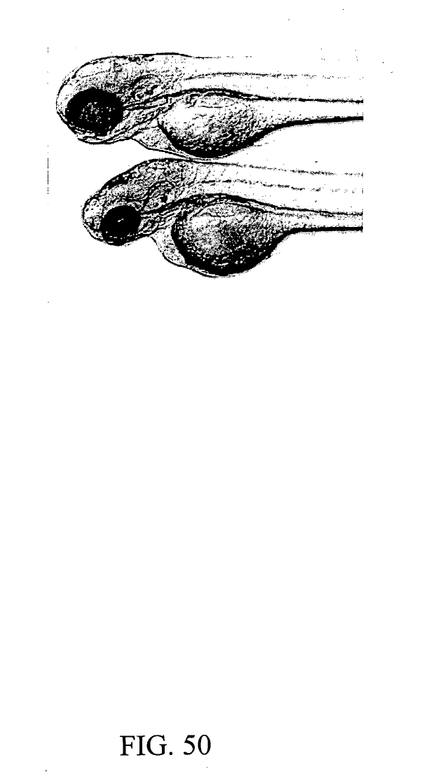

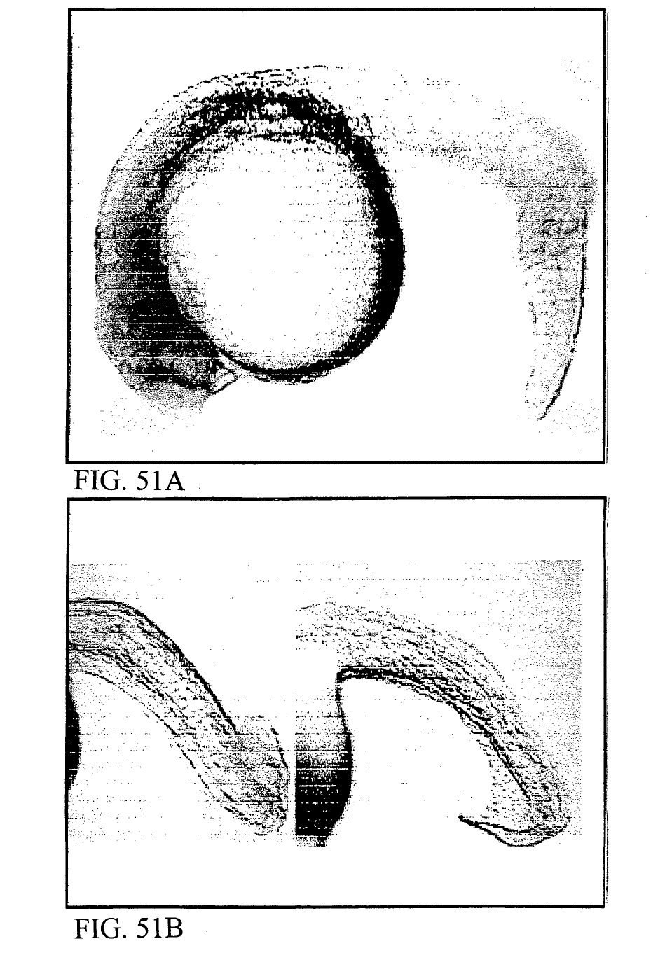

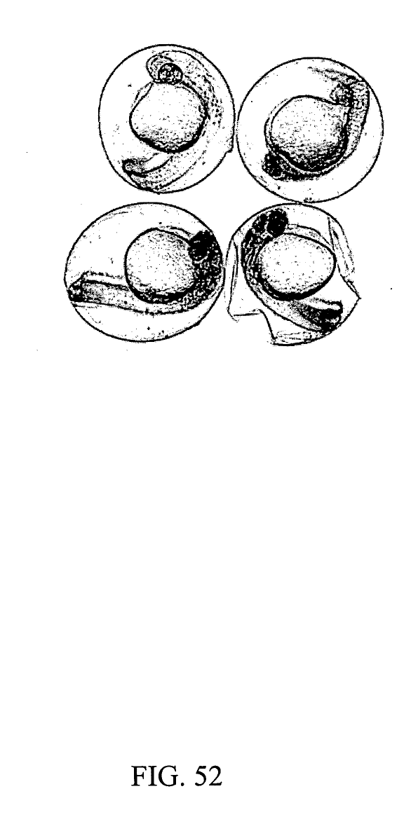

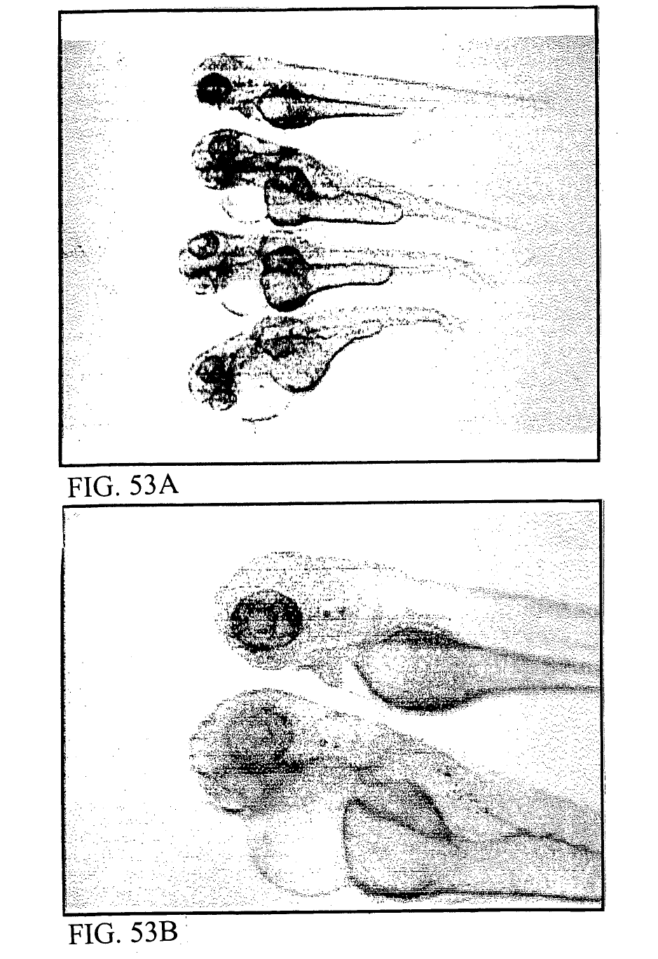

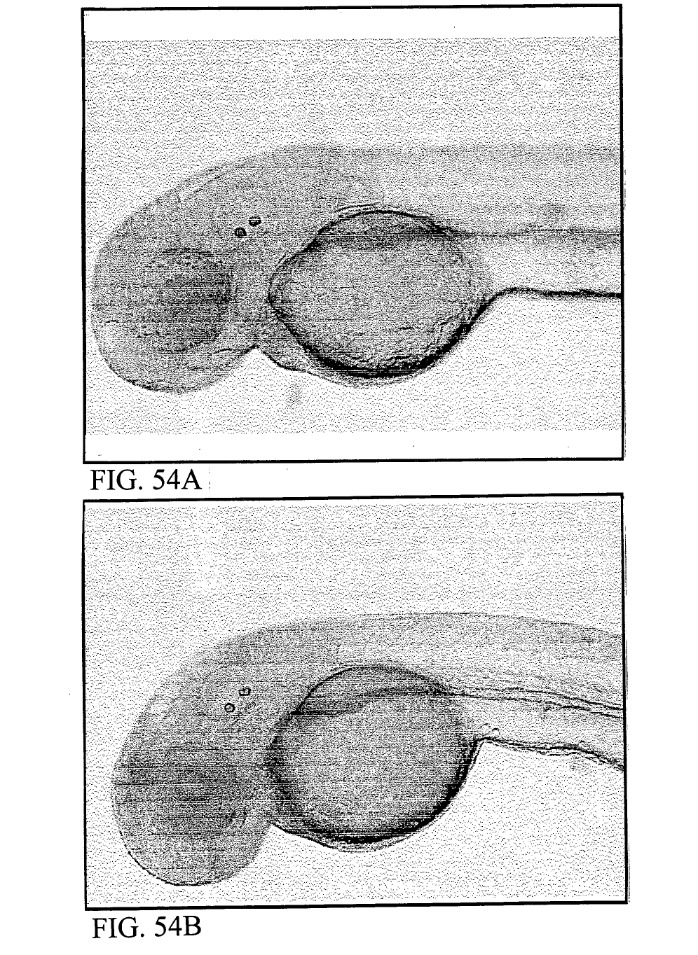

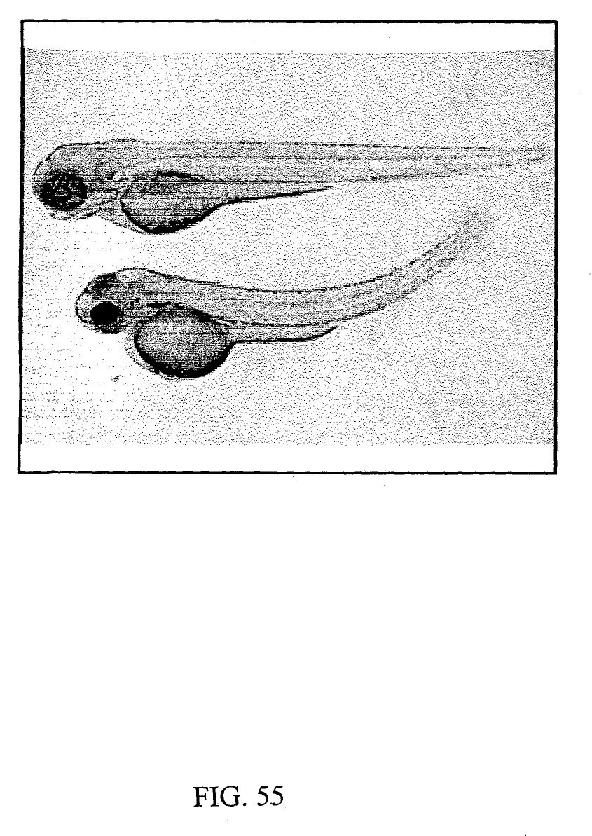

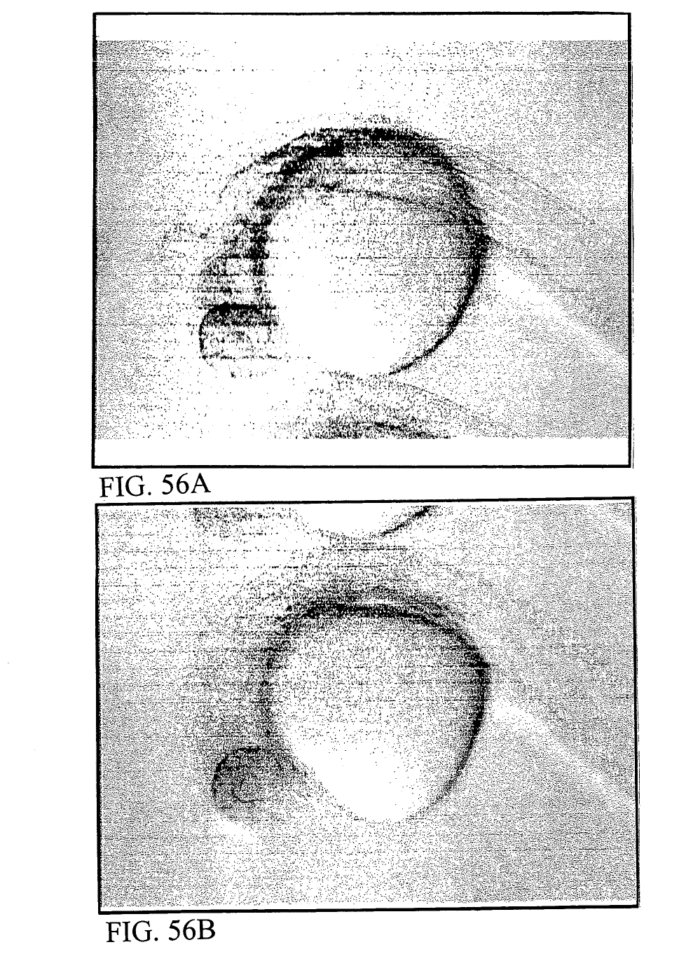

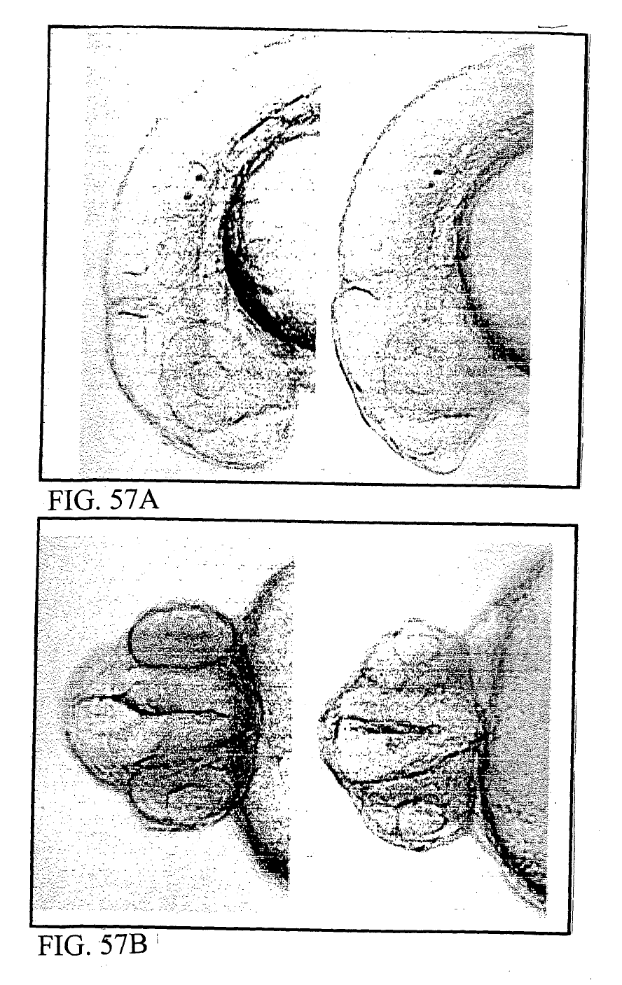

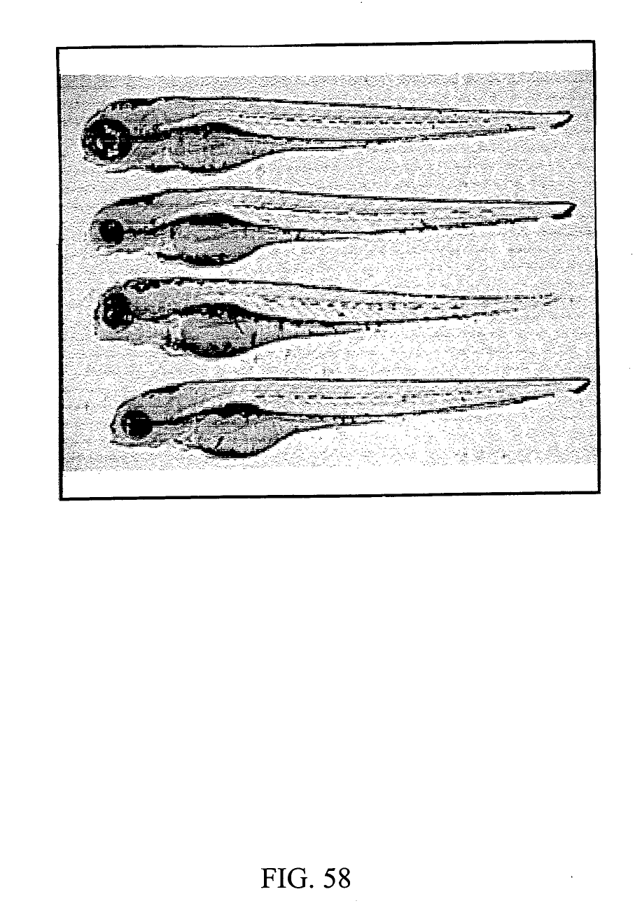

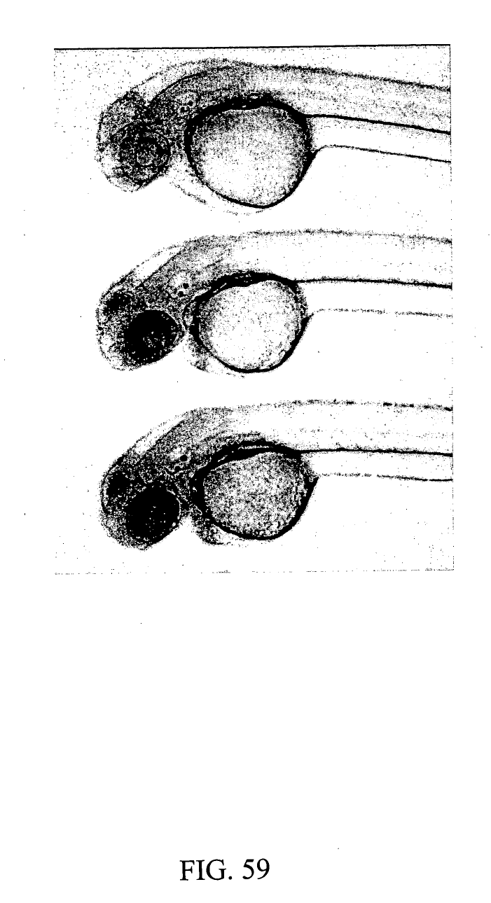

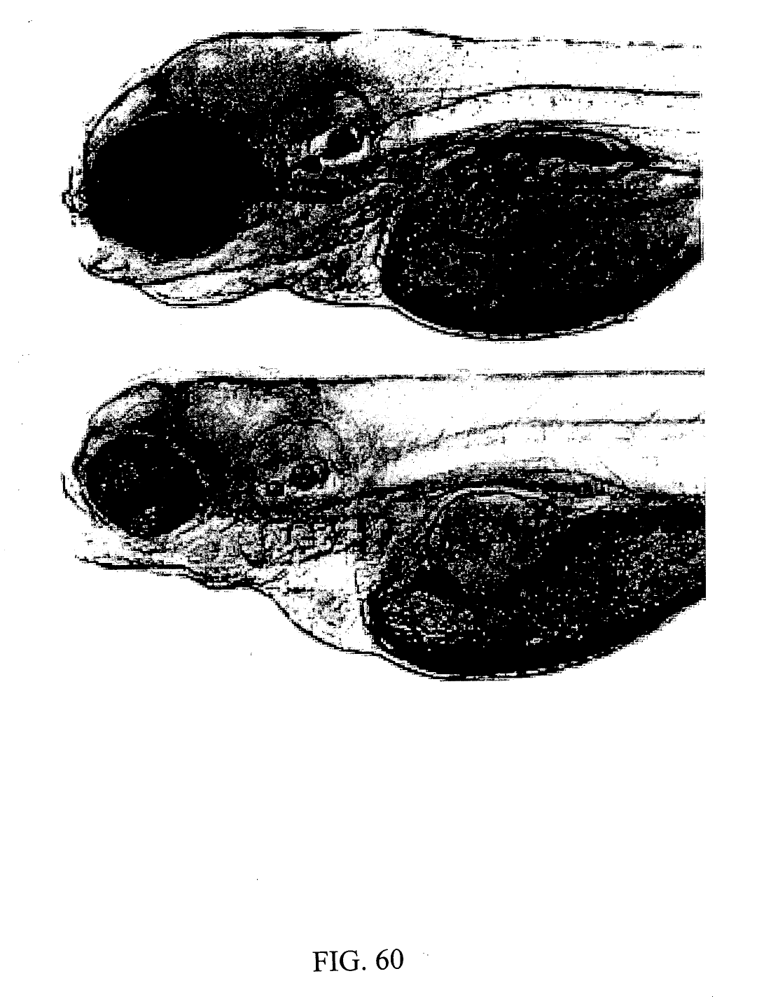

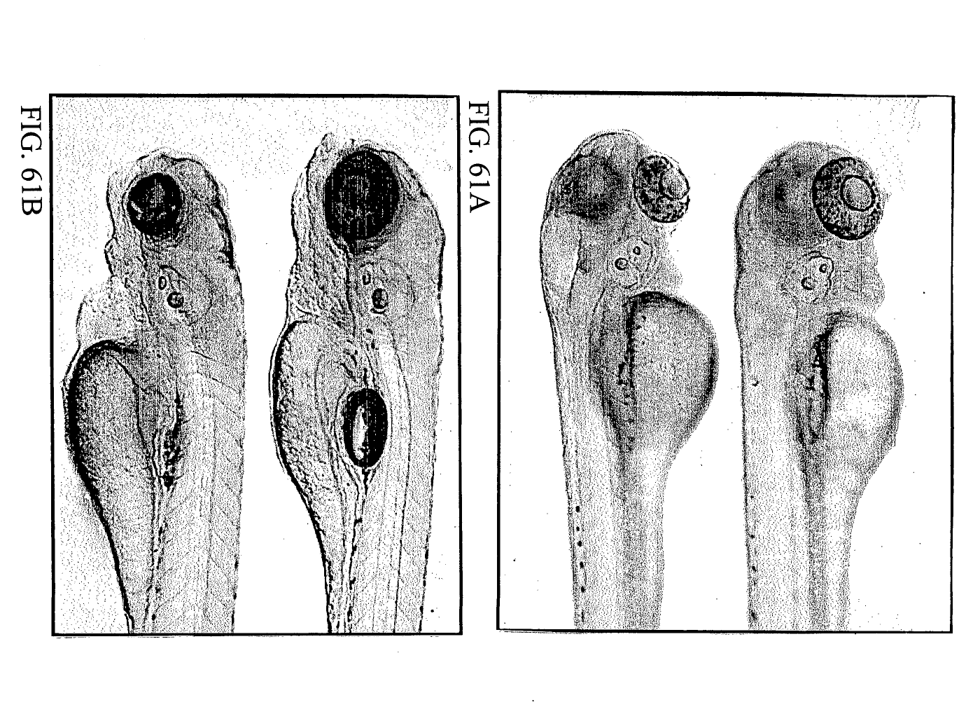

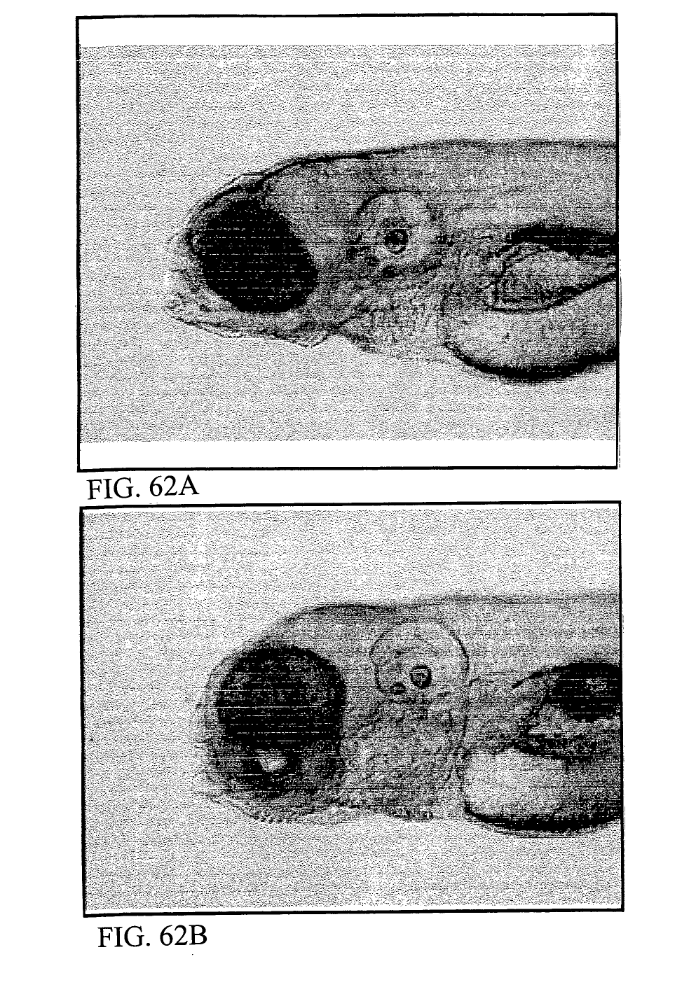

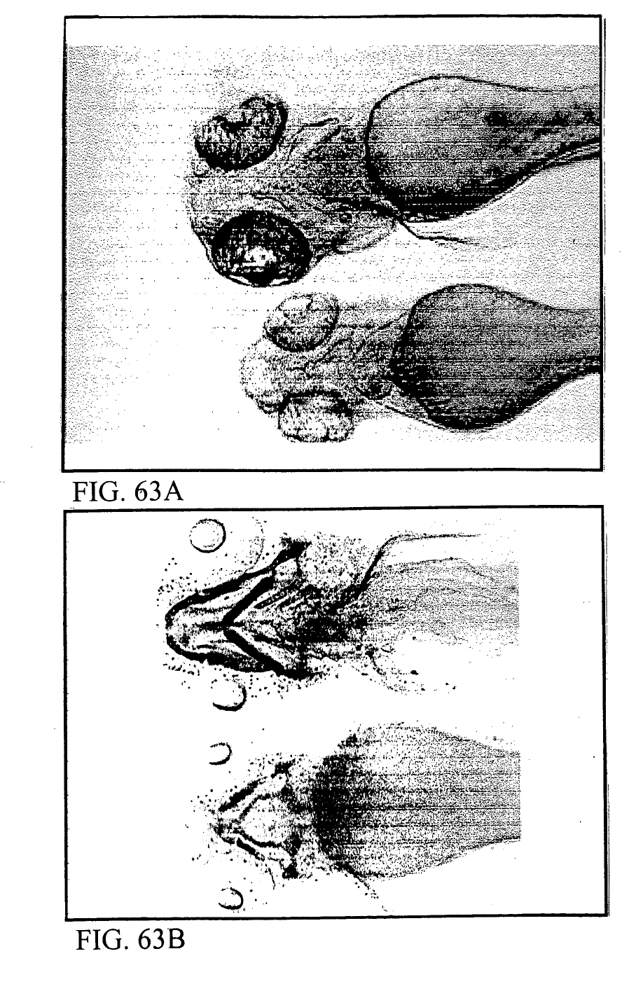

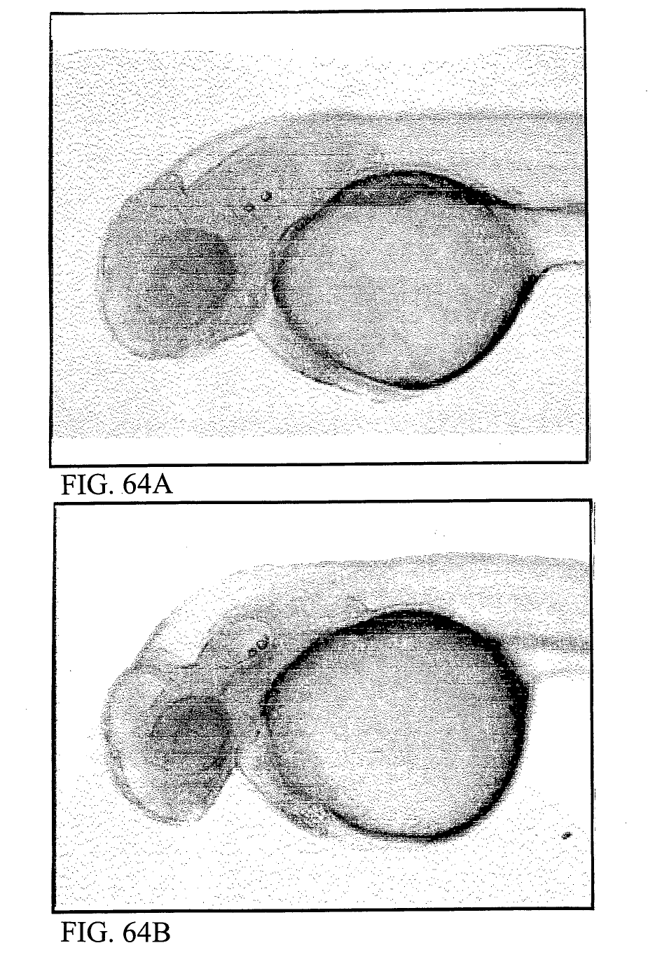

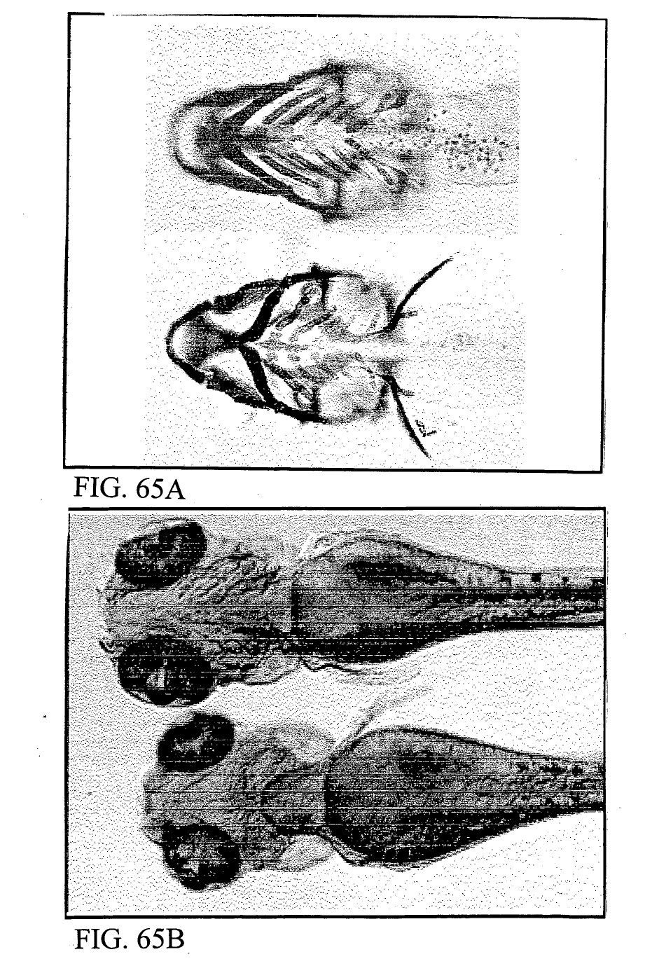

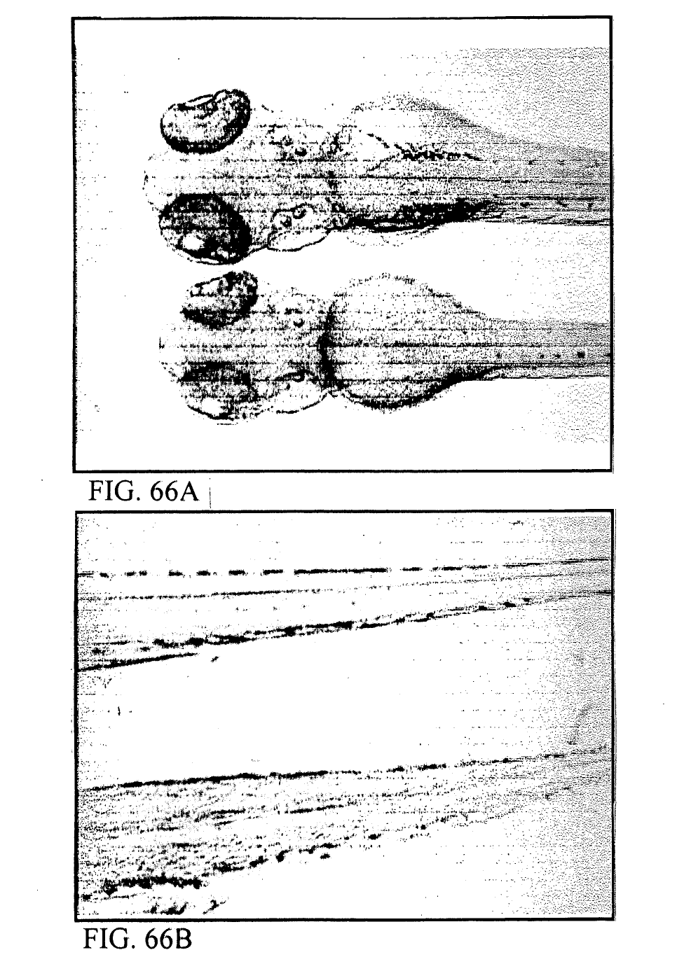

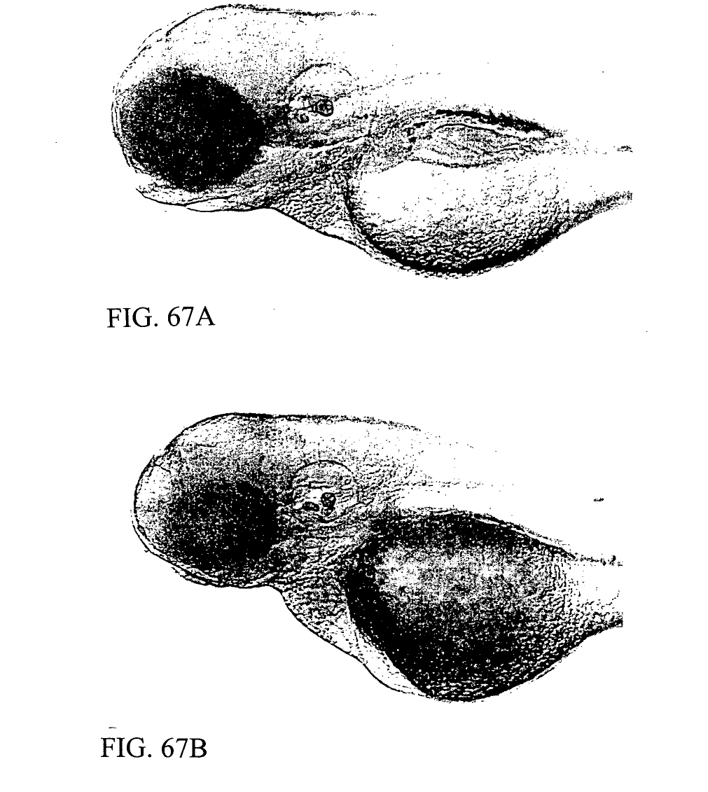

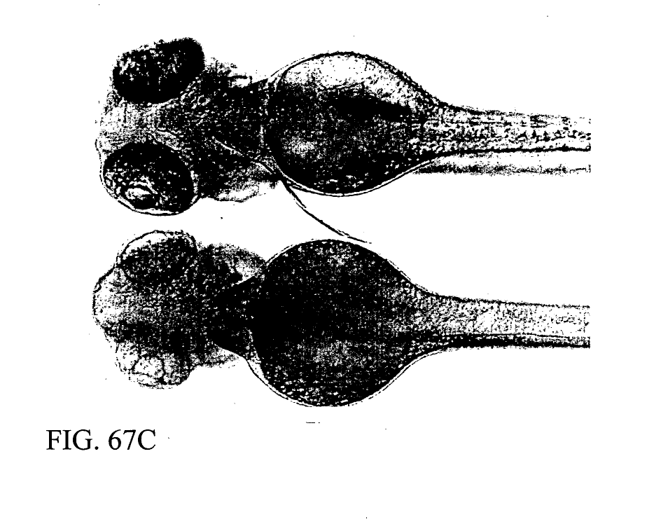

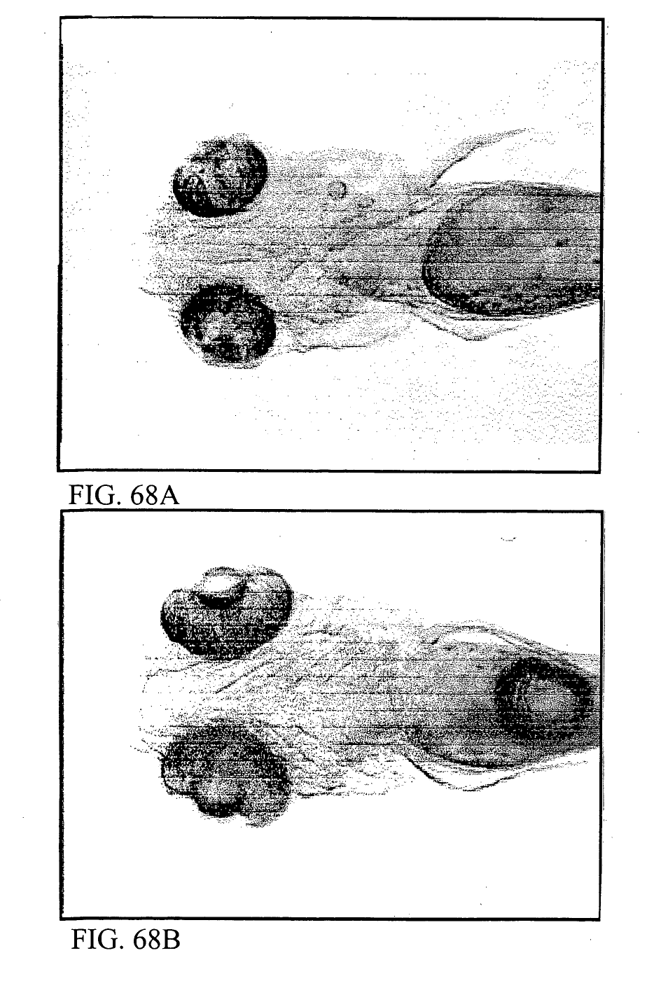

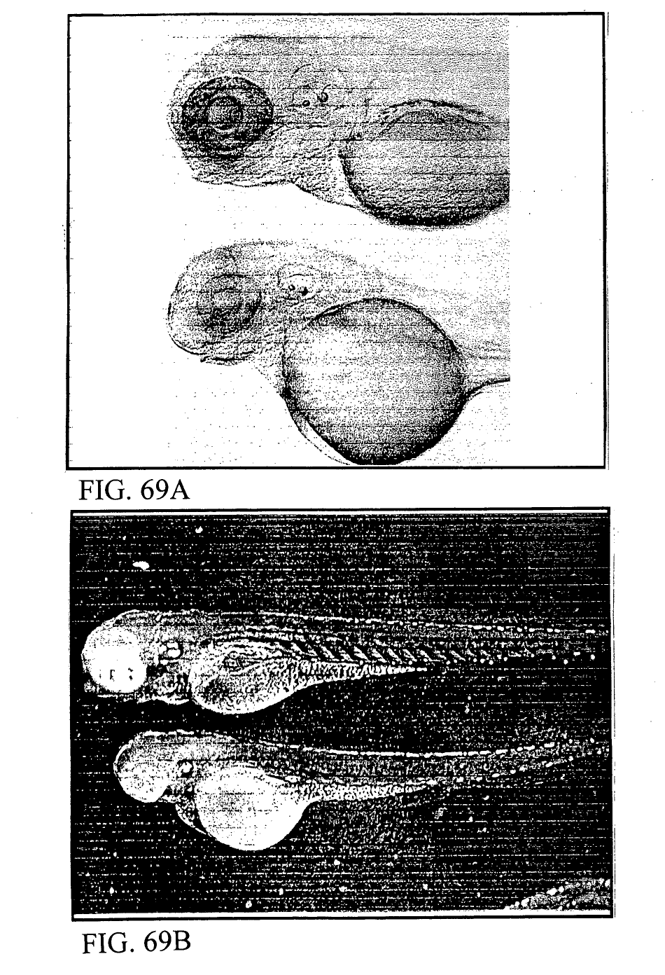

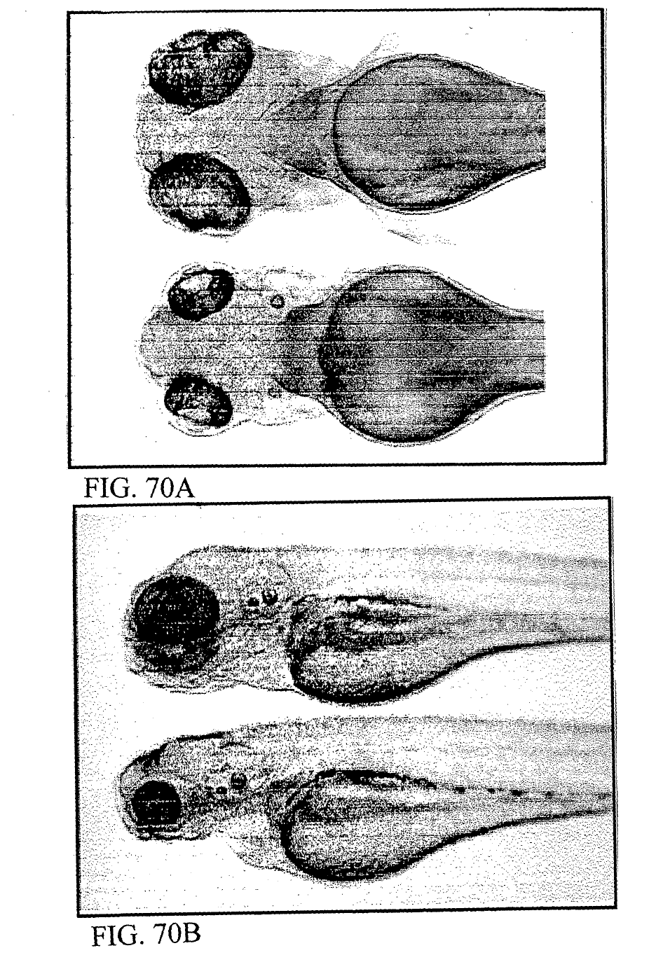

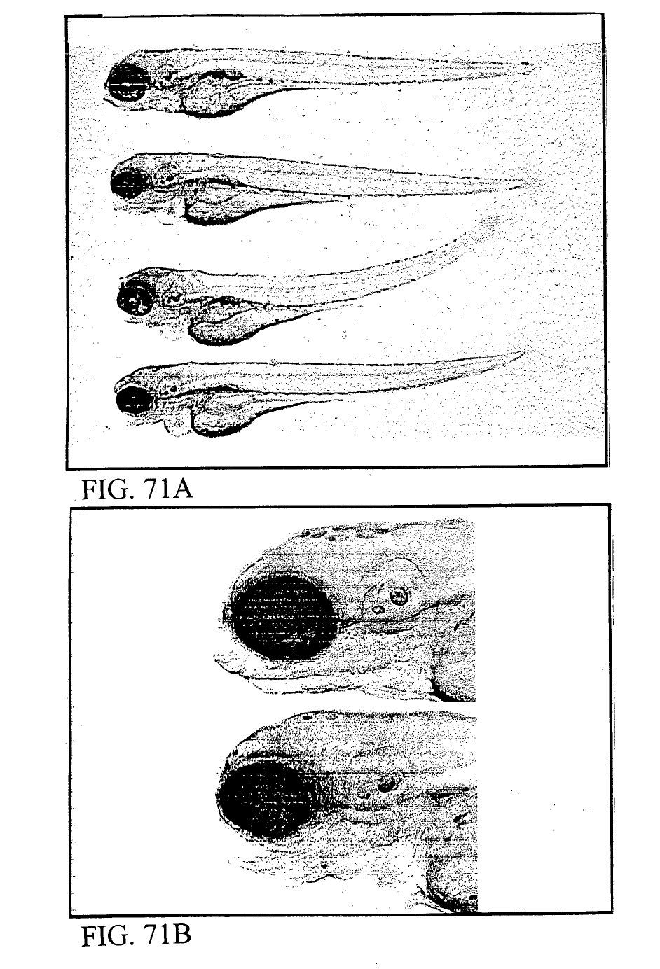

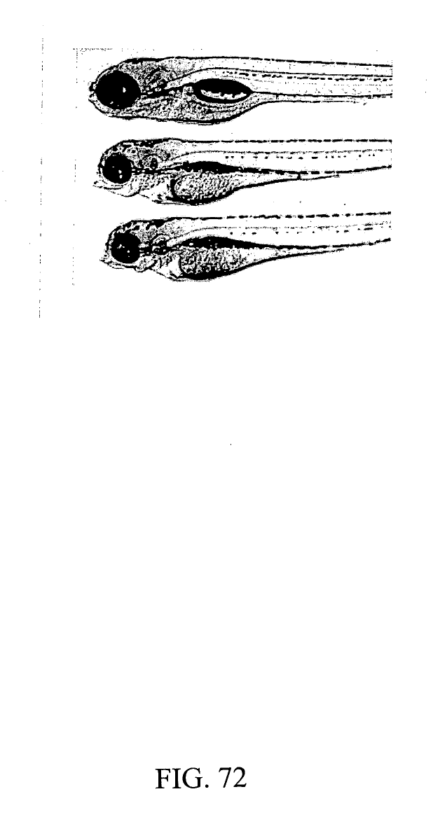

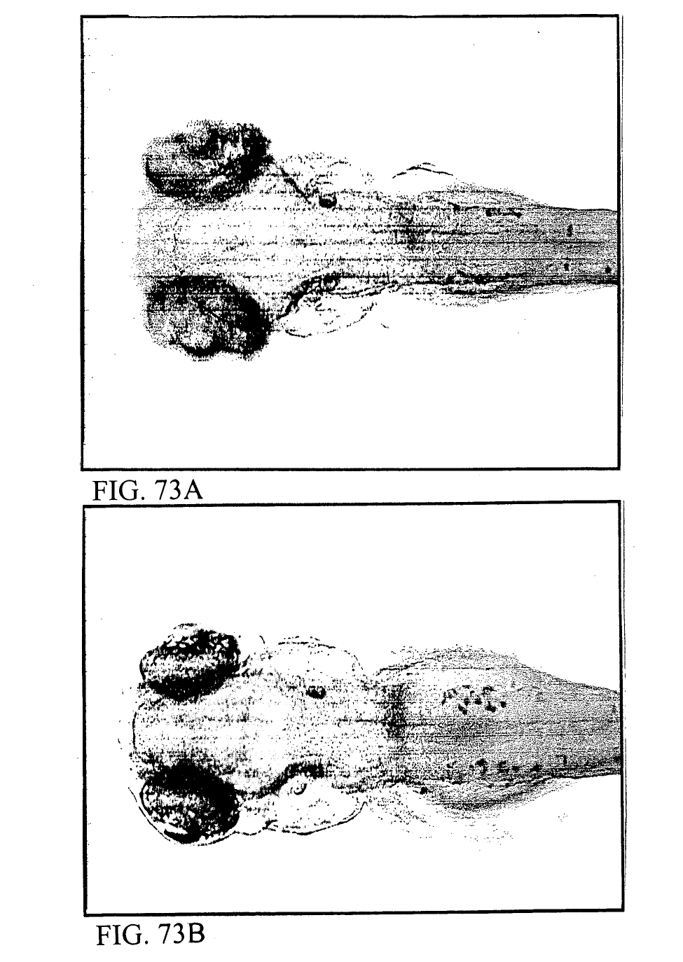

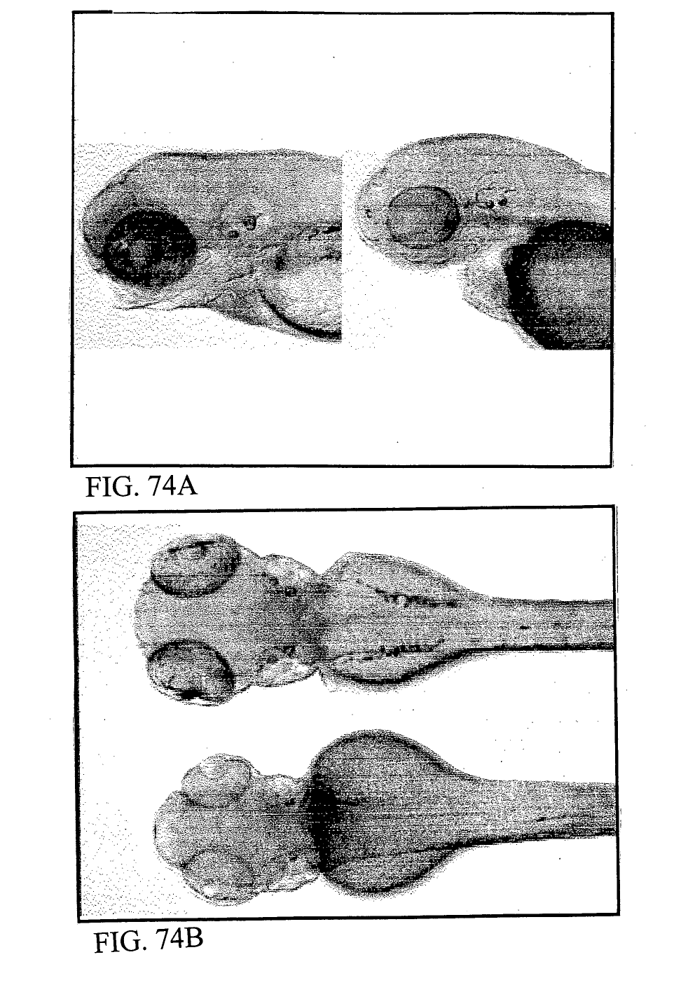

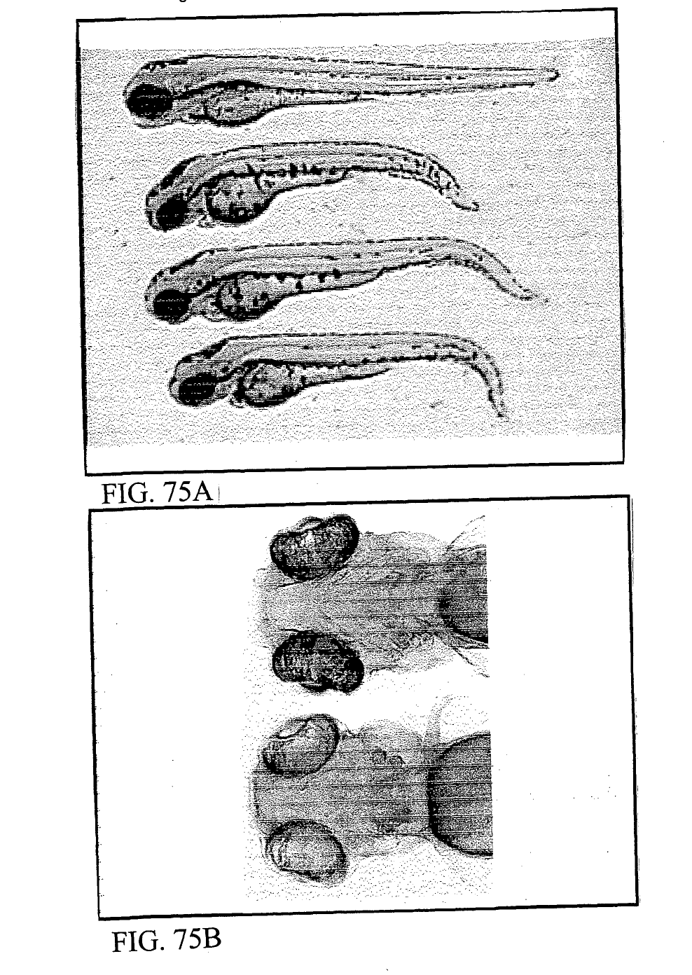

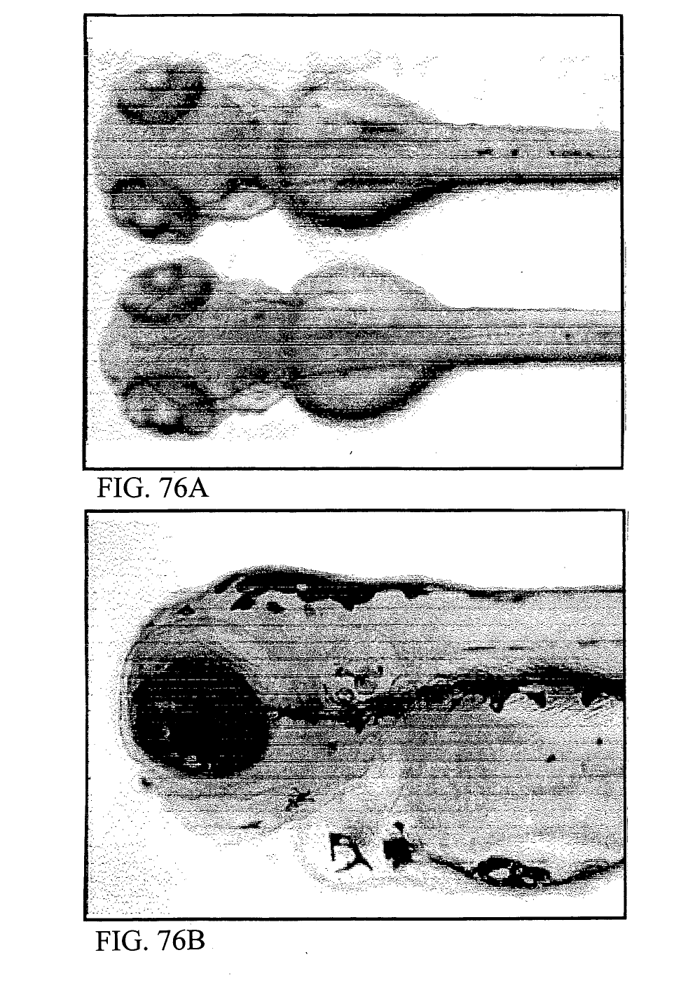

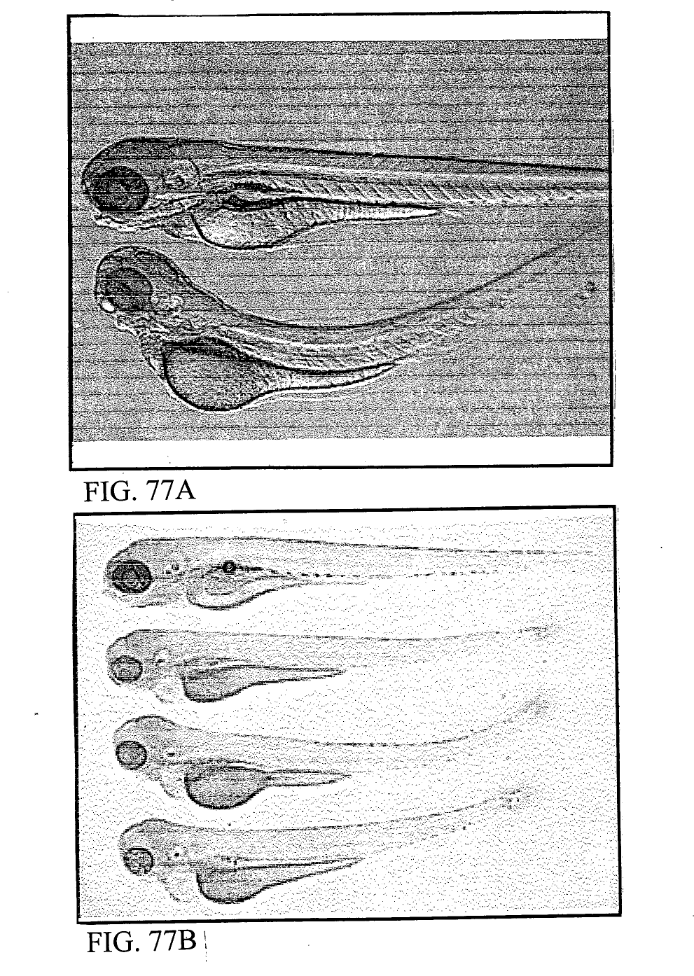

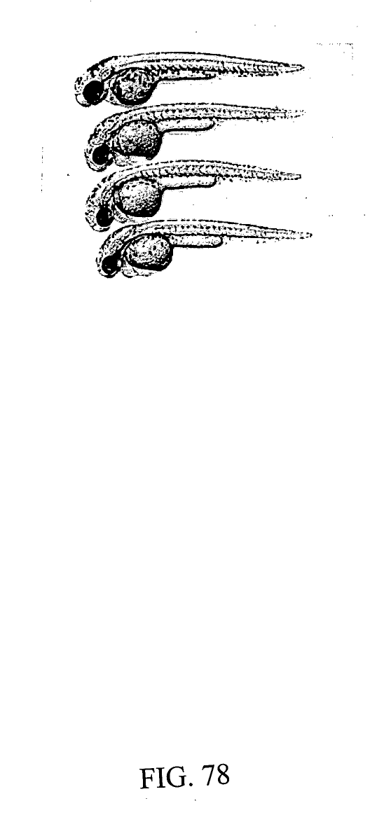

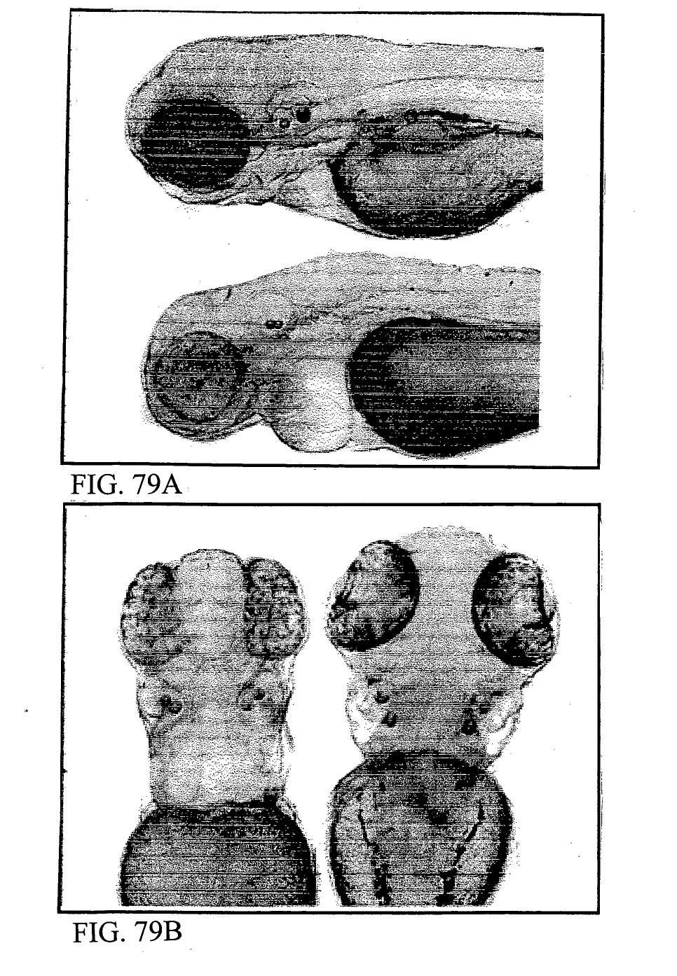

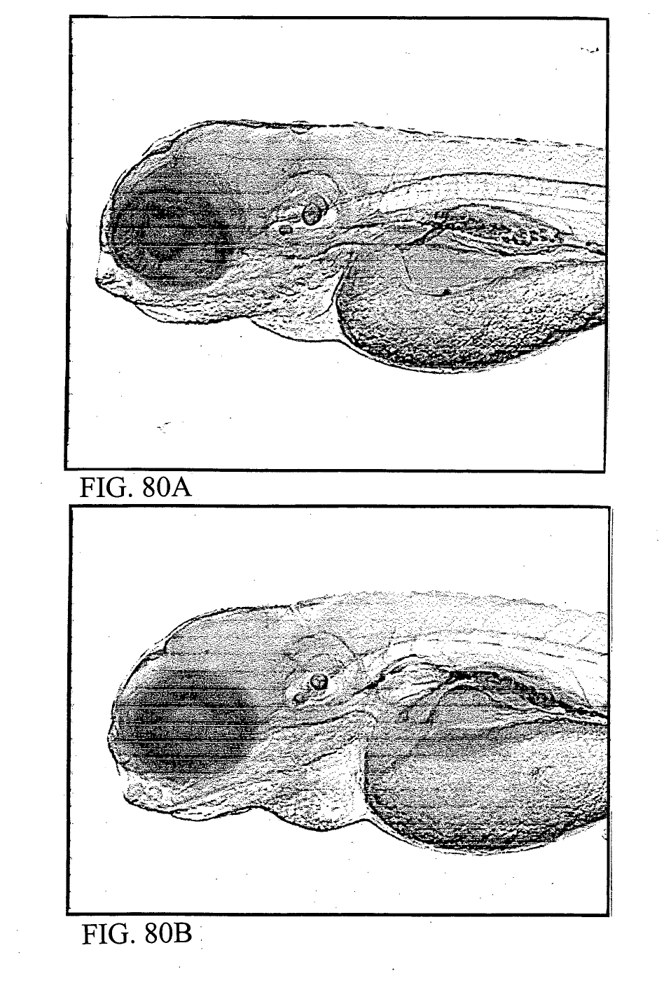

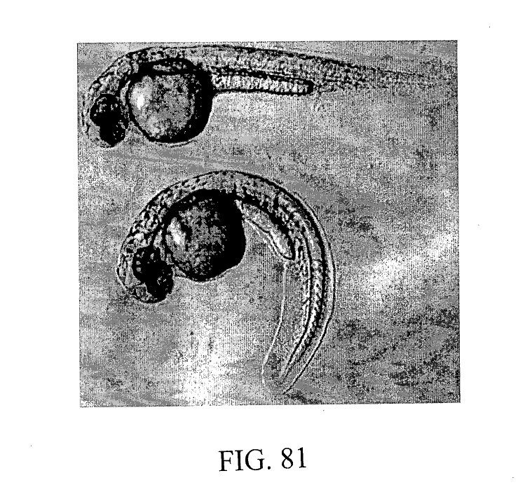

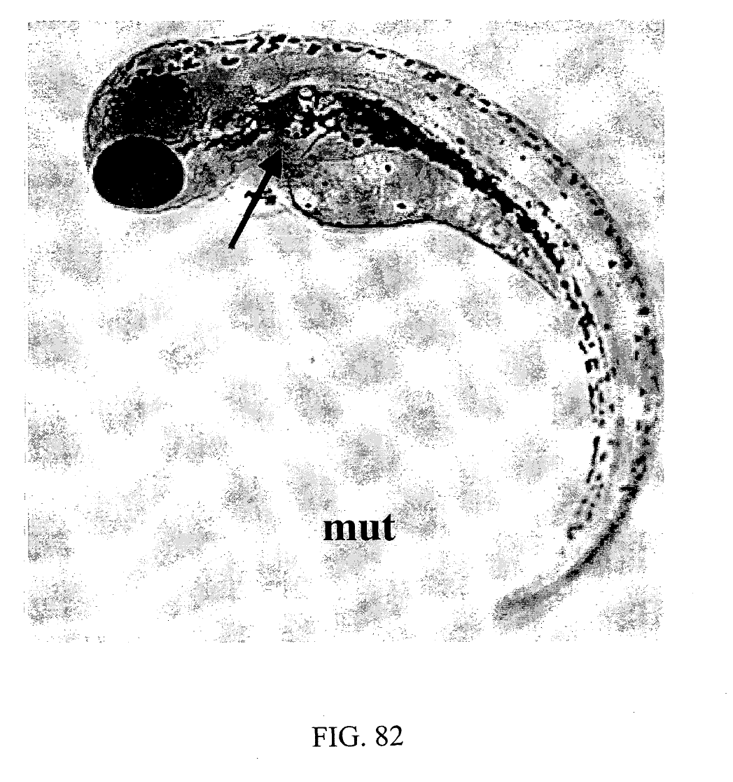

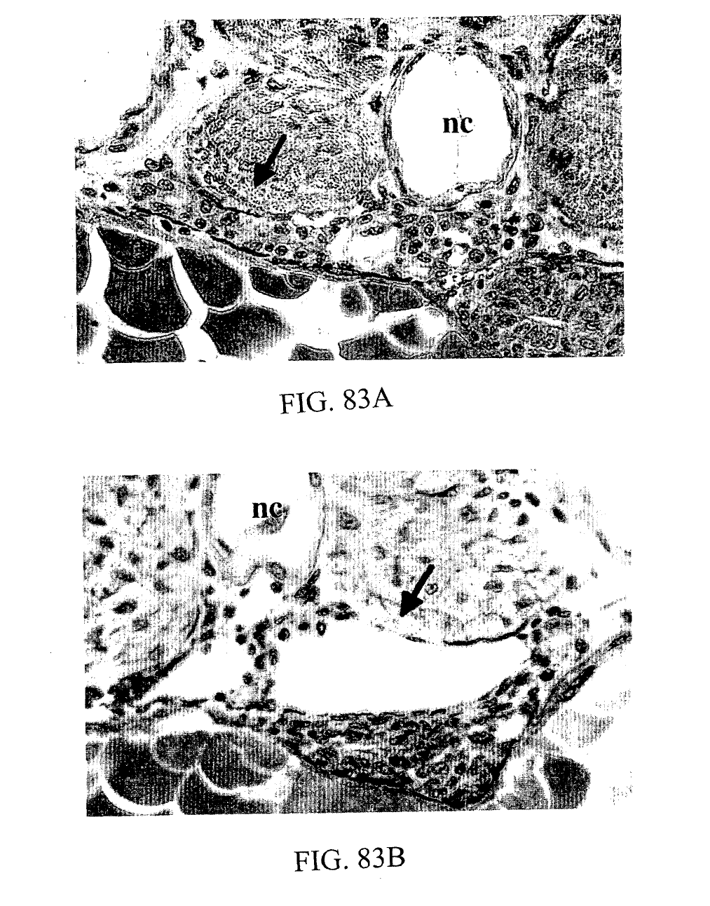

Zebrafish embryos having a mutation in a 459 nucleic acid sequence show apoptosis in the CNS on day one of development (FIG. 33A) and have a curved body by day two of development (FIGS. 33B and 81). In contrast to wild-type embryos of similar age, by day four of development, 459 mutant embryos have kidney tubules that are swollen into cysts (FIGS. 82, 83A, and 83B). Using a polyclonal antibody against the C-terminus of the polypeptide encoded by a 459 nucleic acid sequence, we observed that the 459 polypeptide is localized to the apical surface of the epithelial cells lining the kidney tubule (FIG. 84).

As described below, 459 amino acid and nucleic acid sequences may be used to identify drug targets and may be used to diagnose, prevent, and treat kidney and proliferative disorders using the exemplary methods provided herein.

The Wnt5 (Pipetail) Gene

We isolated zebrafish mutants (alleles hi 1780B and 2735B) containing a viral insertion in the Wnt5 (pipetail) gene (GenBank Accession No. U51268.1). The 1780B allele has an insertion of a GT virus approximately at nucleotide 397, and the 2735B allele has an insertion of a GT virus approximately between nucleotides 530 and 531, of SEQ ID NO:61. The coding region of the Wnt5 (pipetail) gene spans nucleotides 190-1281 of SEQ ID NO:61 and the amino acid sequence of the Wnt5 (pipetail) gene product is provided in SEQ ID NO:62. These mutants have a truncated tail; however, the extent of the truncation is variable.

The Aryl Hydrocarbon Receptor Nuclear Translocator 2A Gene

We isolated zebrafish mutants (alleles hi 1715 and hi 2639C) containing a viral insertion in the Aryl Hydrocarbon Receptor Nuclear Translocator 2A gene (GenBank Accession No. AF155066). The hi 1715 allele has a GT virus inserted approximately at nucleotide 229, and the 2639C allele has a GT virus inserted approximately at nucleotide 240 of SEQ ID NO:63. The amino acid sequence of the zebrafish Aryl Hydrocarbon Receptor Nuclear Translocator 2A gene product is provided in SEQ ID NO:64.

Zebrafish containing a mutation in this gene show very little motility and a minimal tap response by day five of development.

The Vesicular Integral-Membrane Protein VIP 36 Gene

We isolated zebrafish mutants containing an insertion of an F5 virus approximately between nucleotides 219 and 220 of the Vesicular Integral-Membrane Protein VIP 36 gene (SEQ ID NO:65). The coding region of this zebrafish gene spans nucleotides 103 to 1110 of SEQ ID NO:65. The Vesicular Integral-Membrane Protein VIP 36 gene is 76% identical to a human endoplasmic reticulum glycoprotein gene (GenBank Accession No. AAH00347.1) over a region spanning 235 nucleotides, and is 73% identical to this human protein over a region spanning 271 nucleotides, of SEQ ID NO:65. In addition, the zebrafish Vesicular Integral-Membrane Protein VIP 36 gene product is 48% identical and 66% similar to the Canis familiaris homologue over a region spanning 340 amino acids, and is 61% identical and 78% similar to the human DKFZp564L2423 protein (GenBank Accession Nos. NP_{—}110432.1, CAB66552.2, AAH00347.1, AAH05822.1, and AAH05862.1) over a region spanning 311 amino acids of SEQ ID NO:66.

These mutants are touch insensitive at day five of development.

The 299 Gene

We isolated zebrafish mutants containing an insertion of an F5 virus approximately between nucleotides 47 and 48 of the 299 gene (SEQ ID NO:67). The zebrafish 299 gene is 84% identical to a human putative nucleotide binding protein (GenBank Accession No. BC001024) over a region spanning 89 nucleotides of SEQ ID NO:67. In addition, the zebrafish 299 protein is 44% identical and 63% similar to a Homo sapiens estradiol-induced protein (GenBank Accession Nos. AAH01024.1, XP_{—}003213.4, and BAB55169.1) over a region spanning 563 amino acids of SEQ ID NO:68.

Zebrafish mutant for this gene have some apoptosis in the eye and brain, they lack a jaw, branchial arches, and have small fins by the end of day four of development.

The 994 Gene

We isolated zebrafish mutants containing an insertion of a GT virus approximately between nucleotides 66 and 67 of the 994 gene (SEQ ID NO:69). The coding region of the 994 gene may begin at nucleotide 5 or 80 of SEQ ID NO:69. In addition, the zebrafish 994 gene is 35% identical and 49% similar to a Homo sapiens protein (GenBank Accession No. BAB15418.1) over a region spanning 490 amino acids of SEQ ID NO:70.

These mutants have small eyes and a small head. The eyes and head are smaller than wild-type. In addition, zebrafish containing a mutation in the 994 gene have abnormal jaws and arches, as well as an underdeveloped stomach.

The 1373 Gene

We isolated zebrafish mutants (alleles hi 1373 and hi 3245) containing a viral insertion in the 1373 gene. Both the hi 1373 and the hi 3245 alleles have a GT virus inserted approximately between nucleotides 118 and 119 of SEQ ID NO:71. In addition, the coding region of the 1373 gene spans nucleotides 67 to 396 of SEQ ID NO:71. The zebrafish 1373 gene is 82% identical to the Mus musculus Riken Library clone 1110007B08 (GenBank Accession No. AK003520) and is 84% identical to gene encoding the Homo sapiens MGC1346 protein (GenBank Accession No. XM_{—}039445.1) over a region spanning 333 nucleotides of SEQ ID NO:71. Furthermore, the zebrafish 1373 protein is 91% identical and 95% similar to the Arabidopsis thaliana protein F23F1.8 (GenBank Accession No. AAF82203.1) over a region spanning 104 amino acids of SEQ ID NO:72. The zebrafish 1373 protein is also 100% identical to the Homo sapiens MGC 1346 protein (GenBank Accession Nos. NP_{—}116147.1, XP_{—}039445.1, and CAB62939.1) and its Mus musculus homolog (GenBank Accession No. BAB22833.1) over a region spanning 110 amino acids of SEQ ID NO:72.

Mutants in this gene display brain and eye necrosis, constriction of the anterior end of the yolk sac extension, and body curvature by day two of development.

The Denticleless Gene

We isolated zebrafish mutants containing an insertion of an F5 virus approximately between nucleotides 307 and 308 of the Denticleless gene (SEQ ID NO:73). The coding region of the zebrafish Denticleless gene spans nucleotides 34-1974 of SEQ ID NO:73. The zebrafish Denticleless gene is 79% identical to the human gene encoding the L2DTL protein (GenBank Accession No. NM_{—}016448.1) over a region spanning 181 nucleotides of SEQ ID NO:73. The zebrafish Denticleless protein contains four WD-40 domains, which are located between amino acids 88-125, 128-168, 301-339, and 351-383 of SEQ ID NO:74. In addition, this protein is 46% identical and 58% similar to a Homo sapiens protein (GenBank Acession No. BAA91355.1) over a region spanning 729 amino acids of SEQ ID NO:74. Furthermore, the zebrafish Denticleless protein is 66% identical and 77% similar to the N-terminal 386 amino acids of this human protein.

Mutants in this gene have a body that bends upwards, brain necrosis extending down the neural tube, and a wrinkled yolk sac on day one of development. By day two of development, the body shape is convex, the somites are wrinkled, the eye shape is irregular, and the yolk sac extension is absent.

The Ribonucleotide Reductase Protein R2 Gene

We isolated zebrafish mutants (including alleles hi 688 and hi 1706) containing an insertion of a GT virus in the Ribonucleotide Reductase Protein R2 gene (SEQ ID NO:75; GenBank Accession No. U57965). The hi 688 allele has the virus inserted approximately at nucleotide 137 of SEQ ID NO:75, which corresponds to position 360 of an alternatively spliced form of this gene (GenBank Accession No. AW280665). Similarly, the hi 1706 allele has the virus inserted approximately at nucleotide 342 of GenBank Accession No. AW280665. An additional allele has the virus inserted at nucleotide 337 of GenBank Accession No. AW280665. The coding region of the Ribonucleotide Reductase Protein R2 gene spans nucleotides 130-1290 of SEQ ID NO:75 and starts and nucleotide 352 of GenBank Accession No. AW280665. The amino acid sequence of the zebrafish Ribonucleotide Reductase Protein R2 gene product is provided in SEQ ID NO:76.

Zebrafish mutant for this gene show necrosis in the CNS and the entire body curls upward by day two of development.

The TCP-1 Alpha Gene

We isolated zebrafish mutants (alleles hi 491 and hi 1907) containing a viral insertion in the TCP-1 Alpha gene (SEQ ID NO:77; GenBank Accession No. AF143493, with 59 bp added from GenBank Accession No. AW175148). The hi 491 allele results from an insertion of an F5 virus 140 bp upstream of the gene and the hi 1907 allele results from an insertion of an F5 virus approximately between nucleotides 130 and 131 of SEQ ID NO:77. The coding region of the zebrafish TCP-1 Alpha gene spans nucleotides 64-1734 of SEQ ID NO:77 and the amino acid sequence is provided in SEQ ID NO:78.

Zebrafish mutant for the TCP-1 Alpha gene have eyes and a head that are at least 50% smaller than wild-type by day four of development.

The Telomeric Repeat Factor 2 Gene

We isolated zebrafish mutants containing an insertion of a GT virus approximately between nucleotides 529 and 530 of the Telomeric Repeat Factor 2 gene (SEQ ID NO:79). The coding region of the zebrafish Telomeric Repeat Factor 2 gene spans nucleotides 391-2112 of SEQ ID NO:79. In addition the protein encoded by this zebrafish gene is 31% identical and 45% similar to the human Telomeric Repeat Factor 2 protein (GenBank Accession No. U95970) over a region spanning 354 nucleotides, and is 32% identical and 46% similar to this human protein over a region spanning 200 nucleotides, of SEQ ID NO:79. The amino acid sequence of the zebrafish Telomeric Repeat Factor 2 gene product is provided in SEQ ID NO:80.

Zebrafish mutant for this gene have some necrosis in the brain and eye by day two of development.

The SIL Gene

We isolated zebrafish mutants containing an insertion of a GT virus approximately between nucleotide 273 and 274 of the SIL gene. The coding region of the zebrafish SIL gene spans nucleotides 274 to 4059 of SEQ ID NO:81. The zebrafish SIL gene is 79% identical to the DNA sequence encoding the Homo sapiens Tall (SCL) Interrupting Locus (GenBank Accession Nos. NP_{—}003026.1, AAA60550.1, and AAK51418.1) over a region spanning 77 nucleotides, and is 75% identical to this human gene over a region spanning 96 nucleotides, of SEQ ID NO:81. In addition, the zebrafish SIL gene product is 36% identical and 51% similar to the Mus musculus Tall Interrupting Locus gene product (GenBank Accession Nos. NP_{—}033211.1, AAC52386.1, and CAC14001.1) over a region spanning 1348 amino acids of SEQ ID NO:82. In addition, this zebrafish gene product is also 36% identical and 50% similar to the protein encoded by the Homo sapiens SCL Interrupting Locus gene (GenBank Accession Nos. NP_{—}003026.1, AAA60550.1, and AAK51418.1) over a region spanning 1363 amino acids of SEQ ID NO:82.

Zebrafish mutant for the 1262 gene have brain necrosis, a head that is at least 33% smaller than wild-type, and a bent body by day two of development. By day three of development, these zebrafish have a motility defect.

The U1 Small Nuclear Ribonucleoprotein C Gene

We isolated zebrafish mutants containing an insertion of a GT virus approximately between nucleotides 52 and 53 of the U1 Small Nuclear Ribonucleoprotein C gene (SEQ ID NO:83). The coding region of the zebrafish U1 Small Nuclear Ribonucleoprotein C gene spans nucleotides 45 to 521 of SEQ ID NO:83. In addition, the zebrafish U1 Small Nuclear Ribonucleoprotein C gene is 85% identical to the human homologue (GenBank Accession No. XM_{—}004292.3) over a region spanning 170 nucleotides of SEQ ID NO:83. Furthermore, this zebrafish gene is 83% identical and 84% similar to the Xenopus laevis homologue (GenBank Accession No. CAA45354.1) over a region spanning 159 amino acids, and is 84% identical and 85% similar to the human homologue (GenBank Accession Nos. NP_{—}003084.1, XP_{—}043295.1, and CAA31037.1) over a region spanning 159 amino acids, of SEQ ID NO:84.

By day two of development, zebrafish mutant for this gene have a body that is curved upwards, some brain necrosis, motility problems, and smaller otoliths. Furthermore, by day three of development, U1 Small Nuclear Ribonucleoprotein C gene mutant zebrafish have small and irregular eyes, retarded fins, and more pigment in the hind-brain.

The Ski Interacting Protein (SKIP) Gene

We isolated zebrafish mutants containing an insertion of an F5 virus approximately 1.2 kb upstream of the beginning of the zebrafish Ski Interacting Protein (SKIP) gene. The coding region of the zebrafish Ski Interacting Protein (SKIP) gene spans nucleotides 19 to 1626 of SEQ ID NO:85. The nucleic acid sequence encoding zebrafish Ski Interacting Protein is 79% identical to that of the human homologue (GenBank Accession No. U51432) over a region spanning 812 nucleotides of SEQ ID NO:85. In addition, the amino acid sequence of the protein encoded by the zebrafish nucleic acid sequence is 84% identical and 94% similar to that of the human homologue (GenBank Accession No. NP_{—}036377.1) over a region spanning 536 amino acids of SEQ ID NO:86.

Zebrafish mutant for this gene display a curved body, extensive brain necrosis, a lack of brain divisions, and abnormal mobility by day two of development.

The 297 Gene

We isolated zebrafish mutants containing an insertion of an F5 virus approximately at nucleotide 74 of the 297 gene (SEQ ID NO:87). The coding region of the 297 gene may span either from nucleotide 108 to 2153 or from nucleotide 288 to 2153 of SEQ ID NO:87. The 297 gene is 80% identical to the human gene encoding the FLJ10498 protein (GenBank Accession No. XM_{—}011142.2) over a region spanning 173 nucleotides of SEQ ID NO:87. Furthermore, the protein encoded by the 297 gene is 77% identical and 89% similar to the FLJ10498 or AK001360 protein (GenBank Accession Nos: NP_{—}060585.1 and BAA91648.1) over a region spanning 624 amino acids of SEQ ID NO:88.

Zebrafish mutant for the 297 gene show brain necrosis, movement abnormalities, less eye pigment, and a tail that is kinked down. In addition, these mutants are missing the branchial arches, the ethmoid plate, and most of the jaw by day three of development.

The TCP-1 Complex Gamma Chain Gene

We isolated zebrafish mutants (alleles hi 383A and hi 1867) containing a viral insertion in the TCP-1 Complex Gamma Chain gene. The hi 383A allele contains an F5 virus and the hi 1867 allele contains a GT virus inserted approximately between nucleotides 75 and 76 of SEQ ID NO:89. The coding region of the zebrafish TCP-1 Complex Gamma Chain gene spans nucleotides 51 to 1676 of SEQ ID NO:89. The TCP-1 Complex Gamma Chain gene is 76% identical to the Xenopus laevis Chaperonin Subunit CCT Gamma gene (GenBank Accession No. U37062) over a region spanning 1598 nucleotides, and is 75% identical to the Homo sapiens Chaperonin Containing TCP1, subunit 3 (gamma) gene (GenBank Accession No. AAH06501.1) over a region spanning 1573 nucleotides, of SEQ ID NO:89. In addition, this zebrafish protein is 87% identical and 95% similar to the Xenopus laevis homologue over a region spanning 529 amino acids, and is 84% identical and 92% similar to the Homo sapiens homologue over a region spanning 541 amino acids, of SEQ ID NO:90.

Zebrafish having a mutation in this gene have a thinner yolk sac extension.

The Small Nuclear Ribonucleoprotein D1 Gene

We isolated zebrafish mutants containing an insertion of an F5 virus approximately between nucleotides 76 and 77 of the Small Nuclear Ribonucleoprotein D1 gene (SEQ ID NO:91). The coding region of the zebrafish Small Nuclear Ribonucleoprotein D1 gene spans nucleotides 63-420 of SEQ ID NO:91. The zebrafish Small Nuclear Ribonucleoprotein D1 gene is 88% identical to the Mus musculus homologue (GenBank Accession No. NM_{—}009226.1), and is 87% identical to the Homo sapiens homologue (GenBank Accession No. XM_{—}032156.1), over a region spanning 152 nucleotides of SEQ ID NO:91. In addition, the amino acid sequence of the zebrafish Small Nuclear Ribonucleoprotein D1 (SEQ ID NO:92) is 97% identical and 98% similar to the Homo sapiens (GenBank Accession Nos. NP_{—}008869.1, XP_{—}008813.1, XP_{—}032156.1, P13641, A27668, AAA36620.1, and AAH01721.1) or Mus musculus (GenBank Accession Nos. NP_{—}033252.1, AAA96493.1, BAB24092.1, BAB25178.1, BAB27628.1, and BAB28635.1) homologues.

Zebrafish mutant for this gene have an inflated hind-brain and increased necrosis in the CNS, particularly in the eye, when compared to wild-type zebrafish.

The DNA Polymerase Epsilon Subunit B Gene

We isolated zebrafish mutants (alleles hi 783 and hi 1703) containing a viral insertion in the DNA Polymerase Epsilon Subunit B gene. The hi 785 allele contains an insertion of an F5 virus approximately at nucleotide 929, and the hi 1703 allele contains an insertion of a GT virus between nucleotides 1161 and 1162, of SEQ ID NO:93. The coding region of the zebrafish DNA Polymerase Epsilon Subunit B gene spans nucleotides 32 to 1612 of SEQ ID NO:93. The DNA Polymerase Epsilon Subunit B gene is 70% identical to the Mus musculus homologue (GenBank Accession No. AF036898) over a region spanning 1038 nucleotides, is 71% identical to the Xenopus laevis homologue (GenBank Accession No. AB048257) over a region spanning 541 nucleotides, and is 79% identical to the Homo sapiens homologue (GenBank Accession No. AF036899) over a region spanning 96 nucleotides, of SEQ ID NO:93. In addition the protein encoded by the zebrafish DNA Polymerase Epsilon Subunit B gene is 74% identical and 89% similar to the Xenopus laevis homologue (GenBank Accession No. BAB12726.1) over a region spanning 527 amino acids, is 74% identical and 89% similar to the Mus musculus homologue (GenBank Accession No. AAC40045.1) over a region spanning 522 amino acids, and is 73% identical and 87% similar to the Homo sapiens homologue (GenBank Accession Nos. XP_{—}012327.3, P56282, AAC39610.1, and AAK72254.1) over a region spanning 526 amino acids of SEQ ID NO:94.

Zebrafish mutant for this gene show severe necrosis of the brain and eye by day two of development.

The 821-02 Gene

We isolated zebrafish mutants (alleles hi 821-02 and hi 2144) containing a viral insertion in the 821-02 gene. The hi 821-02 allele contains a GT virus inserted approximately between nucleotides 369 and 370, and the hi 2144 allele contains a GT virus inserted approximately between nucleotides 231 and 232, of SEQ ID NO:95. The coding region of the 821-02 gene spans nucleotides 33 to 1982 of SEQ ID NO:95. The zebrafish 821-02 nucleic acid sequence is 76% identical to that encoding the Homo sapiens D26488 protein (GenBank Accession No. BAA05499.1) over a region spanning 99 nucleotides of SEQ ID NO:95. In addition, the zebrafish 821-02 amino acids sequence is 52% identical and 68% similar to the Homo sapiens D26488 amino acid sequence (GenBank Accession No. BAA05499.1) over a region spanning 683 amino acids of SEQ ID NO:96. Furthermore, the zebrafish 821-02 amino acid sequence contains WD40 repeats at positions 105-145 and 148-185 of SEQ ID NO:96.

Zebrafish mutant for this gene have extensive apoptosis in the CNS and the eye by 24 to 48 hours into development, as visualized by acridine orange staining.

The 1045 Gene

We isolated zebrafish mutants containing an insertion of a GT virus approximately between nucleotides 216 and 344 of the zebrafish 1045 gene (SEQ ID NO:97). The coding region of the zebrafish 1045 gene spans nucleotides 2-1039 of SEQ ID NO:97. In addition, the zebrafish 1045 gene is 75% identical to the Homo sapiens serine/threonine kinase 12 sequence (GenBank Accession No. XM_{—}008569.4) over a region spanning 573 nucleotides, and is 75% identical to the Homo sapiens serine/threonine kinase AIE2 sequence (GenBank Accession No. AF054621.1) over a region spanning 511 nucleotides, of SEQ ID NO:97. Furthermore, the protein encoded by the zebrafish 1045 nucleic acid sequence is 73% identical and 86% similar to the Homo sapiens Aurora/Ip11-Related Kinase 3 protein (GenBank Accession No. BAA76292.1), the Homo sapiens STK13 protein (GenBank Accession No. AAC25618.1), and the Homo sapiens Serine/Threonine Kinase AIE2 protein (GenBank Accession No. AAC 25955.1), over a region spanning 266 amino acids of SEQ ID NO:98. The zebrafish 1045 gene product is also 74% identical and 86% similar to Mus musculus homologues (GenBank Accession Nos. BAA04658.1, AAC12683.1, AAH03261.1, and JC4665) over a region spanning 270 amino acids, and is 76% identical and 88% similar to the Rattus norvegicus AIM-1 protein (GenBank Accession No. BAA23794.1) over a region spanning 265 amino acids, of SEQ ID NO:98.

Zebrafish mutant for the 1045 gene have severe brain and head necrosis at 24 hours post fertilization.

The 1055-1 Gene (Zebrafish MAK16 Homologue)

We isolated zebrafish mutants containing an insertion of a GT virus approximately between nucleotides 167 and 168 of the zebrafish 1055-1 gene (SEQ ID NO:99). The coding region of the 1055-1 gene spans nucleotides 152-1204 of SEQ ID NO:99. In addition, the 1055-1 nucleic acids sequence is 74% identical to the DNA sequence encoding a Homo sapiens RNA binding protein (GenBank Accession No. AF251062.1) over a region spanning 552 nucleotides of SEQ ID NO:99. Furthermore, the protein encoded by the zebrafish 1055-1 gene is 70% identical and 83 to 84% similar to Homo sapiens RNA binding proteins (GenBank Accession Nos. NP_{—}115898.1, AAK34952.1, BAB55134.1, XP_{—}050217.1, and XP_{—}050216.1) over a region spanning 285 amino acids of SEQ ID NO:100.

Zebrafish mutant for the 1055-1 gene have a misshapen or missing yolk sac extension and a tail that bends down by day two or three of development. As is noted above, the zebrafish 1055-1 gene product is highly homologous (54% identical and 75% similar over 191 amino acids of SEQ ID NO:100) to the Saccharomyces cerevisiae MAK16 protein (GenBank Accession Nos. AAA34752.1 and AAC05007.1). The yeast protein has been shown to be involved in both cell cycle progression and in the biogenesis of 60S ribosomal subunits.

The Spliceosome Associated Protein 49 Gene

We isolated zebrafish mutants containing an insertion of a GT virus approximately between nucleotides 53 and 54 of the Spliceosome Associated Protein 49 gene (SEQ ID NO:101). The coding region of this zebrafish gene spans nucleotides 20-1216 of SEQ ID NO:101. In addition, the zebrafish Spliceosome Associated Protein 49 gene is 78% identical to the Homo sapiens Splicing Factor 3b, subunit 4 gene (GenBank Accession No. BC004273) over a region spanning 651 nucleotides of SEQ ID NO:101. Furthermore the protein product encoded by the zebrafish Spliceosome Associated Protein 49 gene is 80% identical and 82% similar to the Homo sapiens Splicing Factor 3b, subunit 4 gene product (GenBank Accession Nos. NP_{—}005841.1, XP_{—}001943.1, XP_{—}051919.1, Q15427, A54964, AAA60300.1, and AAH04273.1) over a region spanning 322 amino acids of SEQ ID NO:102.

Zebrafish mutant for this gene have tectal necrosis and a bent body by day two of development.

The DNA Replication Licensing Factor MCM7 Gene

We isolated zebrafish mutants (alleles hi 1411 and hi 2704) containing a viral insertion in the DNA Replication Licensing Factor MCM7 gene. The hi 1411 allele contains an insertion of a GT virus approximately between nucleotides 121 and 122, and the hi 2704 allele contains an insertion of a GT virus approximately at nucleotide 198, of SEQ ID NO:103. The coding region of this zebrafish gene spans nucleotides 93 to 677 of SEQ ID NO:103. However, the DNA sequence encoding the full-length protein may be longer than that of SEQ ID NO:103. One skilled in the art of molecular biology would readily be able to obtain a longer cDNA sequence for this gene, using standard techniques, once provided with the sequence of SEQ ID NO:103.

The zebrafish DNA Replication Licensing Factor MCM7 gene is 74% identical to the Homo sapiens P1cdc47 gene (GenBank Accession No. D55716.1) over a region spanning 286 nucleotides of SEQ ID NO:103 In addition, the protein encoded by this zebrafish gene is 75% identical and 85% similar to the Xenopus laevis CDC47-2p protein (GenBank Accession No. AAC60228.1) over a region spanning 192 amino acids, and is 72% identical and 83% similar to the Homo sapiens DNA Replication Licensing Factor MCM7 (GenBank Accession No. P33993) over a region spanning 194 amino acids, of SEQ ID NO:104.

Zebrafish mutant for this gene have severe necrosis in the eye and in the CNS by late day one/early day two of development.

The DEAD-Box RNA Helicase (DEAD5 or DEAD19) Gene

We isolated zebrafish mutants containing an insertion of a GT virus approximately at nucleotide 132 of the zebrafish DEAD-Box RNA Helicase (DEAD5 or DEAD19) nucleic acid sequence (SEQ ID NO:105). The coding region of this zebrafish nucleic acid sequence spans nucleotides 131 to 1592 of SEQ ID NO:105. In addition, the zebrafish protein (SEQ ID NO:106) encoded by the DEAD-Box RNA Helicase (DEAD5 or DEAD19) nucleic acid sequence contains a DEAD Box between amino acids 113 and 318, as well as a helicase C domain between amino acids 357 and 442). Furthermore, the zebrafish DEAD-Box RNA Helicase (DEAD5 or DEAD19) nucleic acid sequence is 81% identical a human homologue (GenBank Accession No. AJ237946) over a region spanning 810 nucleotides, and is 78% identical to the human sequence with GenBank Accession No. NM_{—}007242.2 over a region spanning 1312 nucleotides, of SEQ ID NO:105. The protein encoded by the DEAD-Box RNA Helicase (DEAD5 or DEAD19) nucleic acid sequence is 84% identical and 92% similar to the Homo sapiens DEAD/H Box Polypeptide 19 and DEAD-Box Protein 5 (GenBank Accession Nos. NP_{—}009173.1, XP_{—}028024.1, XP_{—}028026.1, Q9UMR2, AAH03626.1, and CAB52189.1) over a region spanning 487 amino acids of SEQ ID NO:106.

Zebrafish mutant for this gene show brain necrosis on day one of development and are dead by day two of development.

The 1581 Gene

We isolated zebrafish mutants containing an insertion of a GT virus approximately between nucleotides 346 and 347 of the 1581 nucleic acid sequence (SEQ ID NO:107). The coding region of the zebrafish 1581 nucleic acid sequence spans nucleotides 106 to 912 of SEQ ID NO:107. In addition, the zebrafish 1581 polypeptide may have an RNA recognition motif between amino acids 46 and 118 of SEQ ID NO:108.

The 1581 nucleic acid sequence is 77% identical to a Homo sapiens nucleic acid sequence for a nucleolar protein interacting with the FHA domain of pKi-67 (GenBank Accession No. NM_{—}032390) over a region spanning 165 nucleotides of SEQ ID NO:107. Furthermore, the zebrafish 1581 polypeptide is 48% identical and 62% similar to the Homo sapiens nucleolar phosphoprotein Nopp34 (GenBank Accession Nos. NP_{—}115766.1, XP_{—}037099.1, and BAB41210.1) over a region spanning 273 amino acids of SEQ ID NO:108.

Zebrafish mutant for the 1581 gene show transient brain and eye necrosis between days one and three of development. By day three of development, these mutant zebrafish have heads and eyes that are 50% smaller than those of identically aged wild-type zebrafish.

The Cyclin A2 Gene

We isolated zebrafish mutants containing an insertion of a GT virus approximately at position 374 or 401 of the Cyclin A2 gene (GenBank Accession No. AF234784). The coding region of this zebrafish gene spans nucleotides 250 to 1536 of SEQ ID NO:109 and the amino acid sequence is provided in SEQ ID NO:110.

Zebrafish mutant for the Cyclin A2 gene display eye and CNS necrosis, no jaw development, and abnormal semicircular canals by day three of development. By day five of development, the head is at least 66% smaller than that of a five day-old wild-type zebrafish.

The ISWI/SNF2 Gene

We isolated zebrafish mutants containing an insertion of a GT virus approximately at position 76 of the Imitation Switch ISWI/SNF2 nucleic acid sequence (SEQ ID NO:111). The coding region of this zebrafish gene spans nucleotides 161 to 655 of SEQ ID NO:111. The naturally-occurring zebrafish protein may be considerably longer than the one encoded by SEQ ID NO:111. One skilled in the art of molecular biology would be able to isolate a longer cDNA sequence for the zebrafish ISWI/SNF2 nucleic acid based on the sequence of SEQ ID NO:111, using standard techniques. The zebrafish ISWI/SNF2 nucleic acid sequence is 84% identical to the Xenopus laevis Initiation Switch (ISWI) nucleic acid sequence (GenBank Accession No. AF292095.2) over a region spanning 196 nucleotides, and is 82% identical to the Homo sapiens hSNF2H nucleic acid sequence (GenBank Accession No. AB010882) over a region spanning 224 nucleotides, of SEQ ID NO:111. In addition, the zebrafish ISWI/SNF2 polypeptide is 60% identical and 76% similar to the Xenopus laevis Imitation Switch ISWI polypeptide (GenBank Accession No. AAG01537.2) over a region spanning 145 amino acids, and is 70% identical and 76% similar to the Homo sapiens SWI/SNF related, actin-dependent regulator of chromatin (GenBank Acession Nos. NP_{—}003592.1 and BAA25173.1) over a region spanning 129 amino acids, of SEQ ID NO:112.

On day three of development, zebrafish mutant for the ISWI/SNF2 gene show extensive necrosis in the eye, in particular on the medial 75% of the inner cell ganglion layer and in the optic tectum. By day four of development the eye is at least 25% smaller that that of a four day-old wild-type zebrafish, and the lower jaw has dropped.

The Chromosomal Assembly Protein C (XCAP-C) Gene

We isolated zebrafish mutants containing an insertion of a GT virus approximately between nucleotides 181 and 182 of the zebrafish XCAP-C nucleic acid sequence (SEQ ID NO:113). The coding region of the zebrafish XCAP-C nucleic acid sequence spans nucleotides 192 to 3326 of SEQ ID NO:113. The zebrafish XCAP-C nucleic acid sequence is 78% identical to the Xenopus laevis homologue (GenBank Accession No. U13673.1) over a region spanning 554 nucleotides, is 74% identical to this Xenopus gene over a region spanning 765 nucleotides, and is 72% identical to the Homo sapiens CAP-C nucleic acid sequence (GenBank Accession No. NM_{—}005496.1) over a region spanning 760 nucleotides, of SEQ ID NO:113. In addition, the zebrafish XCAP-C polypeptide sequence is 70% identical and 84% similar to the Xenopus laevis homologue (GenBank Accession Nos. P50532, A55094, and AAA64679.1), and is 65% identical and 81% similar to the Homo sapiens homologue (GenBank Accession Nos. CAB66811.1, NP_{—}005487.1, and BAA73535.1) over a region spanning 979 amino acids of SEQ ID NO:114.

Zebrafish mutant for this gene show necrosis in the optic tectum, eye, and hind-brain by day two of development.

The DNA Replication Licensing Factor MCM2 Gene

We isolated zebrafish mutants (alleles hi 1244 and hi 3205) containing a viral insertion in the DNA Replication Licensing Factor MCM2 nucleic acid sequence. The hi 1244 and hi 3205 alleles are the result of an insertion of a GT virus in the intron preceding nucleotide 399 of SEQ ID NO:115. The coding region of the zebrafish DNA Replication Licensing Factor MCM2 nucleic acid sequence begins with nucleotide 36 of SEQ ID NO:115. In addition, the zebrafish DNA Replication Licensing Factor MCM2 gene is 78% identical to the human homologue (GenBank Accession No. D83987.1) over a region spanning 1164 nucleotides of SEQ ID NO:115. Furthermore, the zebrafish DNA Replication Licensing Factor MCM2 polypeptide is 79% identical and 86% similar to the Homo sapiens MCM2 protein (GenBank Accession Nos. P49736 and BAA12177.1) over a region spanning 893 amino acids of SEQ ID NO:116.

Zebrafish mutant for this gene have small eyes, necrosis in the optic tectum, and abnormal jaws and branchial arches by day five of development.

The DNA Replication Licensing Factor MCM3 Gene

We isolated zebrafish mutants (alleles hi 319 and hi 3068) containing a viral insertion in the DNA Replication Licensing Factor MCM3 nucleic acid sequence. The hi 319 allele is the result of an F5 virus insertion approximately at nucleotide 50, and the hi 3068 allele is the result of a GT virus insertion approximately between nucleotides 75 and 76 of SEQ ID NO:117. In addition, the entire nucleic acid sequence of SEQ ID NO:117 is part of the coding sequence of the DNA Replication Licensing Factor MCM3 gene. The zebrafish DNA Replication Licensing Factor MCM3 nucleic acid sequence is 78% identical to the Mus musculus P1 nucleic acid sequence (GenBank Accession No. X62154.1), and is 77% identical to the Homo sapiens MCM3 nucleic acid sequence (GenBank Accession No. NM_{—}002388.2) over a region spanning 574 nucleotides of SEQ ID NO:117. Furthermore, the zebrafish DNA Replication Licensing Factor MCM3 polypeptide is 83% identical and 93% similar to the Xenopus homologue over a region spanning 178 amino acids, and is 86% identical and 94% similar to the human and mouse homologues (GenBank Accession Nos. NP_{—}002379.2, XP_{—}004096.3, XP_{—}037069.1, XP_{—}037070.1, XP_{—}037068.1, CAA44078.2, CAB75298.1, and AAH01626.1 (human); and CAA44079.1 (mouse)) over a region spanning 167 amino acids, of SEQ ID NO:118.

Zebrafish mutant for this gene have necrosis in the optic tectum on day two of development. By day three of development, no further necrosis is visible in the optic tectum, but the head and eyes are at least 25% smaller than those of three day-old wild-type zebrafish.

The Valyl-tRNA Synthase Gene

We isolated zebrafish mutants containing an insertion of an F5 virus approximately between nucleotides 30 and 31 of the Valyl-tRNA Synthase nucleic acid sequence (SEQ ID NO:119; GenBank Accession No. AF210648). The zebrafish Valyl-tRNA Synthase polypeptide is 67% identical and 75% similar to the Takifugu rubripes homologue (GenBank Accession Nos. P49696 and CAA62967.1) over a region spanning 425 amino acids, and is 52% identical and 62% similar to the Mus musculus homologue (GenBank Accession Nos. ADD26531.1, ADD26532.1, NP_{—}035820.1, Q9Z1Q9, and AAC84151.1) over a region spanning 437 amino acids, of SEQ ID NO:120. This zebrafish polypeptide is also 52% identical and 62% similar to the Homo sapiens homologue (GenBank Accession Nos. CAA41990.1, NP_{—}006286.1, P26640, and AAD21819.1) over a region spanning 440 amino acids of SEQ ID NO:120.

By day three of development, zebrafish with a mutation in this gene have apoptosis in the brain, a smaller head, and lighter, smaller eyes.

The 40S Ribosomal Protein S5 Gene

We isolated zebrafish mutants (alleles 577B (or 577-03) and 1364A) containing a viral insertion in the 40S Ribosomal Protein S5 nucleic acid sequence. The 577B allele is the result of an F5 virus, and the 1364A allele is the result of a GT virus, inserted in an intron between nucleotides 31 and 32 of SEQ ID NO:121. The coding region of the zebrafish 40S Ribosomal Protein S5 nucleic acid sequence spans nucleotides 36 to 645 of SEQ ID NO:121. The zebrafish 40S Ribosomal Protein S5 nucleic acid sequence is 84% identical to that of the human homologue (GenBank Accession Nos. NM_{—}001009.2, XM_{—}034266.1, and XM_{—}034265.1) over a region spanning 593 nucleotides of SEQ ID NO: 121. In addition, the zebrafish 40S Ribosomal Protein S5 is 96% identical and 98% similar to the Mus musculus (GenBank Accession Nos. CAA73041.1, BAB21953.1, BAB26424.1, BAB27113.1, BAB28229.1, BAB28270.1, BAB32115.1, and BAB32203.1) and Homo sapiens (GenBank Accession Nos. NP_{—}001000.2, XP_{—}034266.1, and XP_{—}034265.1) homologues over a region spanning 202 amino acids of SEQ ID NO:122.

Zebrafish mutant for this gene have a swollen “bubble-brain” and motility problems by day two of development. By day three of development, the brain looks relatively normal, but the motility defect persists.

The TCP-1 Beta Gene

We isolated zebrafish mutants (alleles hi 642, hi 1269, and hi 2108) containing a viral insertion in the TCP-1 Beta gene. The hi 642 allele is the result of an F5 virus, and the hi 1269 and hi 2108 alleles are the result of a GT virus, inserted in an intron between nucleotides 63 and 64 of SEQ ID NO:123. The coding region of the TCP-1 Beta nucleic acid sequence includes nucleotides 60-1582 of SEQ ID NO:123. One skilled in the art of molecular biology can isolate additional coding sequence for the zebrafish TCP-1 Beta gene using the sequences provided herein.

In addition, the zebrafish TCP-1 Beta nucleic acid sequence is 73% identical to the Mus musculus Chaperonin Subunit 2 Beta nucleic acid sequence (GenBank Accession No. BC007470) over a region spanning 1353 nucleotides, and is 74% identical the Homo sapiens chaperonin containing TCPI subunit 2 Beta nucleic acid sequence (GenBank Accession No. XM_{—}006861.4) over a region spanning 831 nucleotides, of SEQ ID NO:123. Furthermore, the polypeptide encoded by the zebrafish TCP-1 Beta nucleic acid sequence is 86% identical and 92% similar to the human homologue (GenBank Accession Nos. NP_{—}006422.1, XP_{—}046041.1, XP_{—}006861.4, P78371, AAC96012.1, and AAC98906.1) over a region spanning 507 amino acids of SEQ ID NO:124.

Zebrafish mutant for this gene have jaw and cartilage defects and a small head and eyes by day three of development, when compared to identically aged wild-type zebrafish.

The TCP-1 Eta Gene

We isolated zebrafish mutants (alleles hi 800A and hi 2191) containing a viral insertion in the TCP-1 Eta gene. The hi 800A allele is the result of an F5 virus insertion, and the hi 2191 allele is the result of a GT virus insertion in the intron, present in the genomic sequence, between nucleotides 32 and 33 of SEQ ID NO:125. The coding region of the zebrafish TCP-1 Eta nucleic acid sequence spans nucleotides 31 to 1668 of SEQ ID NO:125. The zebrafish TCP-1 Eta gene is 79% identical to the Mus musculus homologue (GenBank Accession No. BC008255.1) over a region spanning 1584 nucleotides, and is 78% identical to the Homo sapiens homologue (GenBank Accession No. XM_{—}002345.4) over a region spanning 1585 nucleotides, of SEQ ID NO:125. In addition, the zebrafish TCP-1 Eta polypeptide is 87% identical and 94% similar to the Mus musculus homologue (GenBank Accession Nos. NP_{—}031664.1, P80313, S43058, CAA83274.1, BAA81878.1, and AAH08255.1) over a region spanning 541 amino acids, and is 88% identical and 95% similar to the Homo sapiens homologue (GenBank Accession Nos. NP_{—}006420.1, XP_{—}002345.2, Q99832, and AAC96011.1) over a region spanning 532 amino acids, of SEQ ID NO:126. Zebrafish mutant for this gene have small heads and eyes.

The Translation Elongation Factor eEF1 Alpha Gene

We isolated zebrafish mutants containing an insertion of a GT virus in an intron, present in the genomic sequence, between nucleotides 60 and 61 of the Translation Elongation Factor eEF1 Alpha gene (SEQ ID NO:127). The coding region of the zebrafish 1257 gene spans nucleotides 196 to 1326 of SEQ ID NO:127. In addition, the sequence of SEQ ID NO:127 is identical to that of GenBank Accession No. L23807.1, except for the addition of fourteen nucleotides at the 5′end of the sequence.

Zebrafish mutant for this gene have a head and eyes that are at least 33% smaller than those of a comparably aged wild-type zebrafish by day three of development. In addition, these zebrafish mutants display increased apoptosis in the head and eyes, but not in the neural tube, between 48 and 72 hours into development.

The 1257 Gene

We isolated zebrafish mutants containing an insertion of a GT virus approximately at nucleotide 175 of the zebrafish 1257 nucleic acid sequence (SEQ ID NO: 129). The coding region of this zebrafish nucleic acid sequence spans nucleotides 196 to 1326 of SEQ ID NO:129. In addition, the polypeptide encoded by the zebrafish 1257 nucleic acid sequence is 49% identical and 67% similar to the Homo sapiens AK027570 protein (GenBank Accession Nos. BAB55206.1 and XP_{—}002467.3) over a region spanning 371 or 372 amino acids, respectively, of SEQ ID NO:130.

By day four of development, zebrafish mutant for the 1257 gene have a head and eyes that are at least 25% smaller than those of a four day-old wild-type zebrafish. In addition, these mutant zebrafish have an underdeveloped jaw.

The 60S Ribosomal Protein L24 Gene

We isolated zebrafish mutants containing a viral insertion approximately between nucleotides 144 and 145 of the 60S Ribosomal Protein L24 nucleic acid sequence (SEQ ID NO:131). The zebrafish 60S Ribosomal Protein L24 gene is 82% identical to the Gillichthys mirabilis homologue (GenBank Accession No. AF266175) over a region spanning 344 nucleotides, and is 78% identical to the Homo sapiens homologue (GenBank Accession No. NM_{—}000986.1) over a region spanning 363 nucleotides, of SEQ ID NO:131. In addition, the zebrafish 60S Ribosomal Protein L24 is 89% identical and 94% similar to the Homo sapiens (GenBank Accession Nos. NP_{—}000977.1, XP_{—}015463.1, XP_{—}040555.1, P38663, JN0549, AAC28251.1, and AAH00690.1), Rattus norvegicus (GenBank Accession Nos. NP_{—}071960.1, JC2444, and CAA55203.1), Bos taurus (GenBank Accession No. AAC16388.1), and Mus musculus (GenBank Accession Nos. AAH02110.1 and BAB31374.1) homologues over a region spanning 157 amino acids of SEQ ID NO:132.

By day three of development, zebrafish mutant for this nucleic acid sequence have a head and eyes that are at least 33% smaller than those of identically aged wild-type zebrafish.

The Non-Muscle Adenylosuccinate Synthase Gene

We isolated zebrafish mutants (alleles hi 1433 and hi 3081) containing a viral insertion in the Non-Muscle Adenylosuccinate Synthase nucleic acid sequence. The hi 1433 allele is the result of a GT virus insertion in an intron, present in the genomic sequence, between nucleotides 217 and 218, and the hi 3081 allele is the result of a GT virus insertion approximately at nucleotide 209, of SEQ ID NO:133. The coding region of the Non-Muscle Adenylosuccinate Synthase nucleic acid sequence spans nucleotides 16 to 1399 of SEQ ID NO:133. In addition, the zebrafish Non-Muscle Adenylosuccinate Synthase nucleic acid sequence is 76% identical to the Mus musculus Adenylosuccinate Synthase 1, Muscle (Adssl) nucleic acid sequence (GenBank Accession No. NM_{—}007421.1) over a region spanning 333 nucleotides, and is 74% identical to the Homo sapiens ADSS nucleic acid sequence (GenBank Accession No. NM_{—}001126.1) over a region spanning 476 nucleotides, of SEQ ID NO:133. Furthermore, the zebrafish Non-Muscle Adenylosuccinate Synthase polypeptide is 76% identical and 88% similar to Mus musculus (GenBank Accession Nos. BAB23635.1 and BAB26805.1) and Homo sapiens (GenBank Accession No. S21166) homologues over a region spanning 433 and 429 amino acids, respectively, of SEQ ID NO:134.

By day two of development, zebrafish mutant for this gene have a head and eyes that are at least 50% smaller than those of two day-old wild-type zebrafish. In addition, at this time in development, these mutants have some apoptotic cells and lack jaws and branchial arches.

The Nuclear Cap Binding Protein Subunit 2 Gene

We isolated zebrafish mutants containing an insertion of a GT virus in an intron, present in the genomic sequence, between nucleotides 137 and 138 of the Nuclear Cap Binding Protein Subunit 2 nucleic acid sequence (SEQ ID NO:135). The coding region of the zebrafish Nuclear Cap Binding Protein Subunit 2 nucleic acid sequence includes nucleotides 80 to 526 of SEQ ID NO:135. In addition, the zebrafish Nuclear Cap Binding Protein Subunit 2 gene is 74% identical to the Xenopus laevis homologue (GenBank Accession No. X84788) over a region spanning 390 nucleotides, and is 72% identical to the Homo sapiens homologue (GenBank Accession No. NM_{—}007362.1) over a region spanning 420 nucleotides, of SEQ ID NO:135. Furthermore, the zebrafish Nuclear Cap Binding Protein Subunit 2 is 85% identical and 92% similar to the Xenopus laevis homologue (GenBank Accession Nos. P52299, 151602, CAA59259.1, and 2118330B), and is 81% identical and 92% similar to the Homo sapiens homologue (GenBank Accession Nos. XP_{—}003131.3, XP_{—}028278.1, XP_{—}028279.1, P52298, 137222, CAA58962.1, AAH01255.1, and 2118330A) over a region spanning 143 amino acids of SEQ ID NO:136.

Zebrafish mutant for this nucleic acid sequence have some transient necrosis in the CNS between 24 and 48 hours of development, resulting in a head and eyes that are at least 25% smaller than those of identically aged wild-type zebrafish by day four of development. In addition, these mutant zebrafish have an underdeveloped jaw and lack branchial arches three and four. Furthermore, the stomach is also underdeveloped by day five of development.

The Ornithine Decarboxylase Gene

We isolated zebrafish mutants containing an insertion of a GT virus, present in an intron in the genomic sequence, between nucleotides 97 and 98 of the Ornithine Decarboxylase nucleic acid sequence (SEQ ID NO:137). The coding region of the zebrafish Ornithine Decarboxylase nucleic acid sequence spans nucleotides 264 to 1646 of SEQ ID NO:137. This nucleic acid sequence is identical to that of GenBank Accession No. AF290981, except for the addition of 101 nucleotides at the 5′ end. In addition, the amino acid sequence also is provided as SEQ ID NO:138.

By day three of development, zebrafish mutant for this nucleic acid sequence have heads and eyes that are at least 33% smaller than those of identically aged wild-type zebrafish. In addition, these mutants also have underdeveloped jaws and branchial arches, relative to wild-type zebrafish.

The Protein Phosphatase 1 Nuclear Tareting Subunit (PNUTS) Gene

We isolated zebrafish mutants containing an insertion of an F5 virus approximately at nucleotide 303 of the Protein Phosphatase 1 Nuclear Targeting Subunit (PNUTS) nucleic acid sequence (SEQ ID NO:139). The coding region of the zebrafish Protein Phosphatase 1 Nuclear Targeting Subunit (PNUTS) nucleic acid sequence includes nucleotides 322 to 2364 of SEQ ID NO:139. One skilled in the art of molecular biology would be able to identify any additional coding sequence using the sequence provided herein.

In addition, the Zebrafish Protein Phosphatase 1 Nuclear Targeting Subunit (PNUTS) nucleic acid sequence is 76% identical to the Rattus norvegicus Protein Phosphatase 1 Nuclear Targeting Subunit nucleic acid sequence (GenBank Accession No. NM_{—}022951.1) over a region spanning 233 nucleotides, and is 74% identical to Homo sapiens Protein Phosphatase 1 Regulatory Subunit 10 nucleic acid sequence (GenBank Accession No. NM_{—}002714.1) over a region spanning 240 nucleotides, of SEQ ID NO:139. Furthermore, the zebrafish Protein Phosphatase 1 Nuclear Targeting Subunit (PNUTS) polypeptide is 56% identical and 70% similar to the Rattus norvegicus Protein Phosphatase 1 Nuclear Targeting Subunit (GenBank Accession Nos. NP_{—}075240.1 and AAB96775.1) over a region spanning 636 amino acids, and is 55% identical and 68% similar to the Homo sapiens Protein Phosphatase 1 Regulatory Subunit 10 over a region spanning 637 amino acids, of SEQ ID NO:140.

By day three of development, zebrafish mutant for the PNUTS gene have slightly smaller heads. In addition, these mutants curve ventrally, have a slightly compressed jaw, and an underdeveloped gut by day five of development.

The Mitochondrial Inner Membrane Translocating Protein (rTIM23) Gene

We isolated zebrafish mutants containing an insertion of a GT virus approximately at nucleotide 100 of the Mitochondrial Inner Membrane Translocating Protein (rTIM23) nucleic acid sequence (SEQ ID NO:141). The coding region of this zebrafish nucleic acid sequence spans nucleotides 252 to 875 of SEQ ID NO:141. In addition, the zebrafish Mitochondrial Inner Membrane Translocating Protein (rTIM23) nucleic acid sequence is 76% identical to the Rattus norvegicus homologue (GenBank Accession No. NM_{—}019352.1) over a region spanning 416 nucleotides of SEQ ID NO:141. Furthermore, the zebrafish Mitochondrial Inner Membrane Translocating Protein (rTIM23) is 79% identical and 89% similar to the Rattus norvegicus homologue (GenBank Accession Nos. NP_{—}062225.1, JE0154, and BAA21819.1) over a region spanning 190 amino acids of SEQ ID NO:142.

Zebrafish mutant for this gene have lighter eyes than wild-type zebrafish and a circulatory defect in the tail blood vessel on day three of development. In addition, most of these mutant zebrafish are dying by day four of development.

The 1447 Gene

We isolated zebrafish mutants containing an insertion of a GT virus in an intron, present in the genomic sequence, between nucleotides 227 and 228 of the 1447 nucleic acid sequence (SEQ ID NO:143). The coding region of the zebrafish 1447 nucleic acid sequence spans nucleotides 102 to 2687 of SEQ ID NO:143. The zebrafish 1447 nucleic acid sequence is 76% identical to the human sequence that is similar to Riken cDNA clone 2410015A15 (GenBank Accession No. BC005848.1) over a region spanning 910 nucleotides of SEQ ID NO:143. Furthermore, the zebrafish 1447 polypeptide is 59% identical and 73% similar to the Homo sapiens sequence (GenBank Accession No. AAH05848.1) which is similar to the 2410015A15 sequence over a region spanning 738 amino acids of SEQ ID NO:144. The 1447 amino acid sequence also has a DEAD-box motif at amino acids 91 to 288, and a helicase C motif at amino acids 334 to 415, of SEQ ID NO:144, indicating that the zebrafish protein is likely to be an RNA helicase.

By day three of development, zebrafish mutant for this gene have an abnormal jaw, as well as a head and eyes that are at least 25% smaller than those of three day-old wild-type zebrafish. In addition, by day five of development, we observe that these mutants either lack or have severely reduced mandibular and branchial arches. Furthermore, at this time in development, these zebrafish mutant for the 1447 nucleic acid have shorter fins, an underdeveloped stomach, lack a pancreas, and have bent tails.

The ARS2 Gene

We isolated zebrafish mutants (alleles hi 591 and 2765) containing a viral insertion in the ARS2 (Arsenate Resistance Protein 2) nucleic acid sequence. The hi 591 allele is the result of an F5 virus, and the 2765 allele is the result of a GT virus, inserted in an intron, present in the genomic sequence, between nucleotides 103 and 104 of SEQ ID NO:145. The coding region of the zebrafish ARS2 nucleic acid sequence spans nucleotides 141 to 2828 of SEQ ID NO:145. In addition, the zebrafish ARS2 nucleic acid sequence is 75% identical to the Homo sapiens ARS2 nucleic acid sequence (GenBank Accession No. XM_{—}005015.4) over a region spanning 614 nucleotides of SEQ ID NO:145. In addition, the zebrafish ARS2 polypeptide sequence is 69% identical and 79% similar to that of the human homologue (GenBank Accession No. AAK21005.1) over a region spanning 917 amino acids of SEQ ID NO:146.

Zebrafish mutant for the ARS2 gene have necrosis in the tectum and an underdeveloped jaw by day three of development. In addition, by day five of development, these mutants have flecks of pigment in the otoliths and widespread edema.

The Sec61 Alpha Gene

We isolated zebrafish mutants (alleles hi 1058 and hi 2839B) containing a viral insertion in the Sec61 Alpha nucleic acid sequence (GenBank Accession Nos. AY029527 (nucleotide) and AAK40295 (amino acid)). Both the hi 1058 and the hi 2839B alleles are the result of GT virus insertions in an intron, present in the genomic sequence, between nucleotides 132 and 133 of SEQ ID NO:147. The coding region of the zebrafish Sec61 Alpha nucleic acid sequence spans nucleotides 126 to 1556 of SEQ ID NO:147 and the amino acid sequence is provided in SEQ ID NO:148.

Zebrafish mutant for the Sec61 Alpha nucleic acid sequence have widespread defects by day three of development, including a bent body, a head and eyes that are at least 25% smaller than those of identically aged wild-type zebrafish, and no development of the jaw or branchial arches.

The BAF53a Gene

We isolated zebrafish mutants containing an insertion of a GT virus approximately at nucleotide 160 of the BAF53a nucleic acid sequence (SEQ ID NO:149). The coding region of this zebrafish nucleic acid sequence spans nucleotides 110 to 1396 of SEQ ID NO:149. In addition, the Zebrafish BAF53a nucleic acid sequence is 77% identical to the human homologue (GenBank Accession No. XM_{—}011050.2) over a region spanning 1288 nucleotides of SEQ ID NO:149. The amino acid sequence encoded by the zebrafish BAF53a nucleic acid sequence is 88% identical and 94% similar to the Mus musculus (GenBank Accession No. AAH01994.1) and Homo sapiens (GenBank Accession Nos. NP_{—}004292.1, XP_{—}011050.2, XP_{—}37377.1, XP_{—}37379.1, XP_{—}37376.1, XP_{—}37378.1, AAC94991.1, BAA74577.1, and AAH01391.1) homologues over a region spanning 429 amino acids of SEQ ID NO:150.

Zebrafish mutant for this nucleic acid sequence have a body that is curved sideways, a small, underdeveloped eye, and an enlarged ventricle with edema by day one of development.

The Histone Deacetylase Gene

We isolated zebrafish mutants (alleles hi 1618 and hi 2628) containing a viral insertion in a Histone Deacetylase nucleic acid sequence. The hi 1618 allele is the result of a GT virus insertion approximately at nucleotide 88 of SEQ ID NO:151, and the hi 2628 allele is the result of a GT virus insertion in an intron, present in the genomic sequence, between nucleotides 98 and 99 of SEQ ID NO:151. The coding region of this zebrafish Histone Deacetylase nucleic acid sequence spans nucleotides 46 to 1485 of SEQ ID NO:151. In addition, the zebrafish Histone Deacetylase gene is 81% identical to the Xenopus laevis Deacetylase (RPD3) gene (GenBank Accession No. AF020658.1) over a region spanning 1406 nucleotides, is 79% identical to the Homo sapiens Histone Deacetylase 1 gene (GenBank Accession No. BC000301.1) over a region spanning 1245 nucleotides, and is 77% identical to the Homo sapiens Histone Deacetylase 2 gene (GenBank Accession No. XM_{—}004370.4) over a region spanning 1253 nucleotides, of SEQ ID NO:151. Furthermore, the amino acid sequence encoded by this zebrafish histone deacetylase nucleic acid sequence is 90% identical and 96% similar to a likely Xenopus laevis Histone Deacetylase 1-2 (GenBank Accession No. 042227) over a region spanning 481 amino acids, is 87% identical and 92% similar to Homo sapiens (GenBank Accession No. XP_{—}004370.4) or Mus musculus (GenBank Accession Nos. NP_{—}032255.1 and P70288) Histone Deacetylase 2 over a region spanning 483 amino acids, of SEQ ID NO:152.

Zebrafish mutant for this Histone Deacetylase nucleic acid sequence have multiple developmental defects. By 48 hours into development, zebrafish mutant for this gene have an enlarged heart, with the atrium being at least twice the size of that of identically aged wild-type embryos. In addition, the eyes are at least 33% smaller than those of wild-type zebrafish and the ears lack semicircular canals and have otoliths that are either very close together or fused. Furthermore, by day three of development, the fin buds are not growing and the jaws and branchial arches are not visible. Finally, by day five of development, Alcian blue fails to stain any cartilage corresponding to the pectoral fins, jaw or branchial arches, or the neurocranium.

The Fibroblast Isoform of the ADP/ATP Carrier Protein Gene

We isolated zebrafish mutants containing an insertion of an F5 virus in an intron, present in the genomic sequence, between nucleotides 178 and 179 of the fibroblast isoform of the Fibroblast Isoform of the ADP/ATP Carrier Protein nucleic acid sequence (SEQ ID NO:153). The coding region of this zebrafish nucleic acid sequence spans nucleotides 68 to 961 of SEQ ID NO:153. The zebrafish Fibroblast Isoform of the ADP/ATP Carrier Protein gene is 83% identical to the Xenopus laevis Adenine Nucleotide Translocase (Ant1) GenBank Accession No. AF231347) over a region spanning 886 nucleotides, and is 82% identical to the Homo sapiens Solute Carrier Family 25 Adenine Nucleotide Translocator 5 (GenBank Accession No. NM_{—}001152.1) over a region spanning 870 nucleotides, of SEQ ID NO:153. Furthermore, the amino acid sequence encoded by this zebrafish nucleic acid sequence is 93% identical and 96% similar to the Xenopus laevis Adenine Nucleotide Translocase (GenBank Accession No. AAF63471.1) sequence over a region spanning 298 amino acids, and is 93% identical and 97% similar to the Homo sapiens Solute Carrier Family 25 Adenine Nucleotide Translocator 5 (GenBank Accession Nos. NP_{—}001143.1, P05141, AAA51737.1, and AAB39266.1) sequence over a region spanning 296 amino acids, of SEQ ID NO:154.

Zebrafish mutant for the Fibroblast Isoform of the ADP/ATP Carrier Protein nucleic acid sequence fail to inflate their swim bladder, but have no other readily apparent phenotypes.

The TAFII-55 Gene

We isolated zebrafish mutants containing an insertion of a GT virus in an intron, present in the genomic sequence, between nucleotides 107 and 108 of the TAFII-55 nucleic acid sequence (SEQ ID NO:155). The zebrafish TAFII-55 nucleic acid sequence is 75% identical to the Mus musculus TATA Box Binding Protein Associated Factor (GenBank Accession No. NM_{—}011901.1) spanning 553 nucleotides, and is 74% identical to the Homo sapiens TATA Box Binding Protein Associated Factor (GenBank Accession No. XM_{—}003757.2) over a region spanning 559 nucleotides, of SEQ ID NO:155. In addition, the amino acid sequence encoded by the zebrafish TAFII-55 nucleic acid sequence is 71% identical and 83% similar to the sequence of the Homo sapiens TATA Box Binding Protein Associated Factor (GenBank Accession Nos. XP_{—}003757.1, NP_{—}005633.2, XP_{—}049114.1, Q15545, CAA66636.1, and AAK30585.1), and is 68% identical and 81% similar to the sequence of the Mus musculus TATA Box Binding Protein Associated Factor (GenBank Accession Nos. NP_{—}036031.1 and AAD46767.1), over a region spanning 336 amino acids of SEQ ID NO:156.

Zebrafish mutant for the TAFII-55 nucleic acid sequence fail to inflate their swim bladder. In addition, by day five of development, these mutants have heads that are approximately 20% smaller, and have eyes that are approximately 33% smaller, than those of identically aged wild-type zebrafish.

The above experiments were carried out using the following materials and methods.

### Materials and Methods

Zebrafish Strain Maintenance

Zebrafish were raised and maintained as described previously (Culp et al., Proc. Natl. Acad. Sci. USA 88:7953-7957, 1991), with the following exceptions. Synchronized eggs for injection were obtained by placing four females and two males (which had been separated the night before) in a 4 liter mating chamber for 10-15 min. Pair matings for raising F1 and F2 fish and for screening F3 embryos were performed in 1 liter mating chambers as described by Mullins et al. (Curr. Biol. 4:189-202, 1994). Paramecia fed to fry were counted and delivered in measured amounts three times a day; a total of approximately 400 paramecia per fry per day were required between days 5 and 7 and 800 paramecia per fry per day between days 8 and 11 to allow fish to reach a size at which they could eat brine shrimp.

Founder fish were generated from embryos from either of two lethal-free lines that were obtained as follows: We crossed outbred fish originally from the laboratory of Dr. Christianne Nüsslein-Volhard (Tübingen, Germany), but carried in our laboratory for approximately 6 years with AB*, a line selected by Charlene Walker (University of Oregon) as highly suitable for use in haploid and early pressure screens. We raised families from each of 15 pair matings. Sibling matings within each family were performed to identify families with no embryonic lethal mutations. Two lines, designated TAB-5 and TAB-14, were identified (no embryonic mutations seen in 18 matings from TAB-5 or in 22 matings from TAB-14) and used to obtain embryos for virus injections.

Virus Preparation and Injection

A packaging cell line 293 gp/bsr (Miyoshi et al., Proc. Natl. Acad. Sci. USA 94:10319-10323, 1997), grown in Dulbecco's modified Eagle medium supplemented with fetal calf serum, penicillin, streptomycin, and fungisome, was infected with SFGnlslacZ virus (Gaiano et al., Proc. Natl. Acad. Sci. USA 93:7777-7782, 1996) at three multiplicities of infection (M.O.I.s), 0.05, 0.5, and 5. Four days later, cells were trypsinized and stained with the vital stain fluorescein di-β-D-galactopyranoside (FDG), and passed through a cell sorter. Moderate and highly fluorescent cell populations from each of the three cell populations were selected, grown for 1 week, and then cloned. A total of 46 clones were screened to identify the one capable of producing the highest titer of virus following calcium phosphate-mediated transfection with the plasmid pHCMV-G (Yee et al., Proc. Natl. Acad. Sci. USA 91:9564-9568, 1994), which encodes the envelope protein of vesicular stomatitis virus. The medium was changed 24 hr after transfection, and collections of supernatant were made at 48, 72, 96, and 120 hours. The titer of virus harvested at these time points was measured using mouse 3T3 cells. Titers ranged from 0 to 5.4×10⁶ CFU/ml. The 10 best lines were selected and viral supernatants were titered on a fish cell line, PAC2. The F5 line, derived from an infection at a multiplicity of infection (M.O.I.) of 5, and harboring a single proviral genome, was selected and used to produce F5 virus for the experiment.

Large quantities of virus stocks were prepared by calcium phosphate transfection of F5 cells that had been seeded 1 day earlier on fifteen 15 cm tissue culture plates treated with 0.01% poly-L-lysine. A total of 50 μg of pHCMV-G DNA per plate was used in the transfection. Media were changed at 24 hr post-transfection, collected at 48, 72, and 96-hr post-transfection were filter sterilized (0.2 pm filter) and concentrated by centrifugation at 21,000 rpm with an SW28 rotor for 1.5 hr at 4° C. (Burns et al., Proc. Natl. Acad. Sci. USA 90:8033-8037, 1993). Viral pellets were resuspended in 30 μl of PBS, the titer determined, and either used fresh or frozen at −80° C. for future use. We estimate that approximately 250,000 embryos were injected on approximately 230 days over a period of 12 months.

Embryo Assay

To determine whether viral stocks had high titers on embryos and to ensure that founder fish that were raised were efficiently infected, we determined the proviral DNA content of several injected embryos from every batch injected using an assay designated the embryo assay.

For the bulk of the project, this assay was performed by quantitative Southern analysis. Ten injected embryos were lysed as five pools of two at 3-5 days of age in 100 mM Tris (pH 8.3), 200 mM NaCl, 5 mM EDTA, 0.4% SDS, 100 pg/ml proteinase K and lysed overnight. DNA was precipitated with ethanol, resuspended, and digested with PvuII, which cuts several times in the viral sequence. The samples were then subjected to electrophoresis through 0.8% agarose and Southern blots were performed along with a reference control from a fish with one proviral insert. The Southern blots were then hybridized with probes to the provirus and to the zebrafish RAG2 gene. The amount of DNA present in each band recognized by a probe was determined using a Molecular Dynamics Phosphoimager, the virus/RAG2 ratio calculated and normalized to the internal reference of 1. Subsequently, the embryo assay was performed using real time quantitative PCR. Single embryos were lysed at 2 days of age and processed as described below for the fin clips.

Identification of F1 Fish Carrying Multiple Proviral Insertions

We raised 30 fish per F1 family. To identify fish with at least three unique proviral inserts, we proceeded as follows. At 8-10 weeks of age, fish were anesthetized and placed on a small piece of PARAFILM® flexible thermoplastic film, and the end of their caudal fins were amputated with a scalpel and placed in wells of a 96-well plate. The fish were stored in disposable 16-oz (473 ml) cups while the fin clips were processed. DNA was extracted by incubation in 50 pl of ELVIS lysis buffer (50 mM KCI, 10 mM Tris at pH 8.5, 0.01% gelatin, 0.45% NP-40, 0.45% Tween-20, 5 mM EDTA, 200 pg/ml proteinase K) for at least 2 hr at 55° C. The Proteinase K was inactivated by placing the samples at 96° C. for 15 minutes.

Approximately 1 pl from each sample served as template for real time quantitative PCR with a Perkin-Elmer 7700 Sequence Detector (Heid et al., Genome Res. 6:986-994, 1996). Primers and the probe used to amplify viral sequences are as follows: SFG F, 5′-CGCTGGAAAGGACCTTACACA-3′; SFG R, 5′-TGCGATGCCGTCTACTTTGA-3′, and SFG probe, 5′-FAM-CTGCTGACCACCCCCACCGC-TAMRA-3′. A separate primer/probe combination was utilized for an internal reference amplicon to amplify the RAGI locus (RAG F, 5′-ATTGGAGAAGTCTACCAGAAGCCTAA-3′; RAG R, 5′-CTTAGTTGCTTGTCCAGGGTTGA-3′, RAG probe, 5′JOE-GCGCAACGGCGGC-GCTC-TAMRA-3′). The SFG primers and RAG primers were used at final concentrations of 74 and 150 nM, respectively, whereas both RAG and SFG probes were used at 200 nM. Each reaction was carried out in a final volume of 12.5 pl with Perkin-Elmer Master Mix. The cycling profiles were 2 minutes at 50° C., 10 minutes at 95° C., and 30×(15 seconds at 95° C., 1 minute at 60° C.). Each 96-well run contained six wells of a reference control from a fish with six inserts. At the end of each run, the RAG and SFG Cts (threshold cycle; the cycle at which the amount of product passed a certain threshold in the linear amplification range) were calculated for each sample and a ACt value was defined by subtracting the SFG Ct from the RAG Ct. The larger the ACt, value the greater the number of viral insets for any given sample. By subtracting the average six-insert fish ACt from each sample's ACt, we calculate the AACt, which can then be used in the following formula to estimate the number of inserts per fish: n=6×2AAct.

The eight fish with the highest ACt values from each F1 family were further analyzed by Southern blot to allow selection of fish with the greatest number of unique inserts among this group. The remaining fin clip sample for these fish was digested with BglII, which cuts only once in the provirus, and subjected to electrophoresis through 0.8% agarose for approximately 1200 volt-hours before being subjected to Southern analysis. The Southern Blots were then incubated with probes that yield one band per insert. Only fish with at least three unique inserts were kept and used to generate F2 families.

Identification of Mutagenic Insertions and Gene Cloning

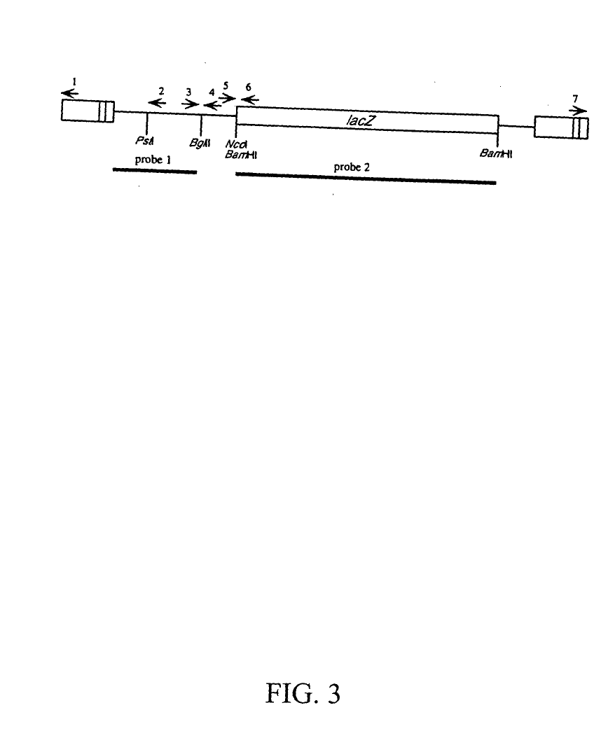

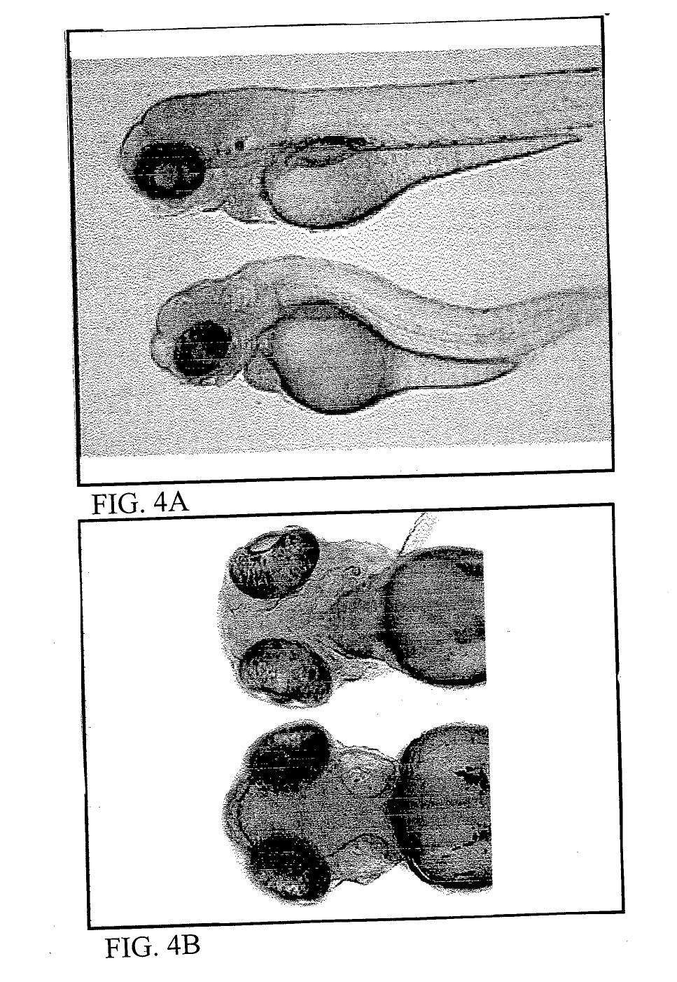

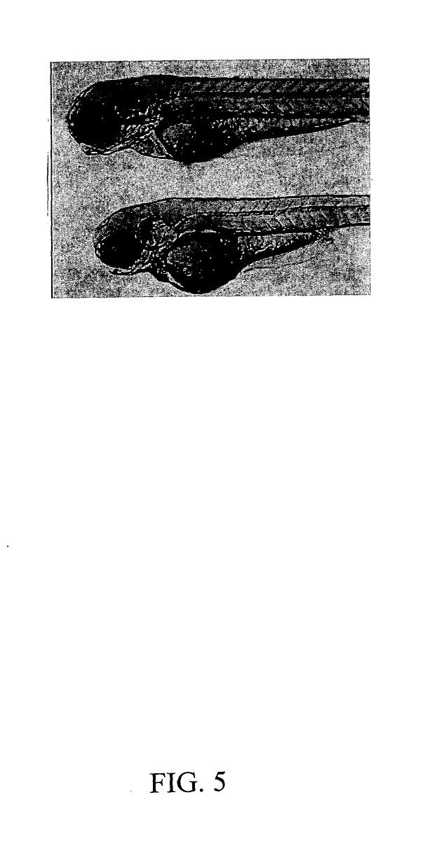

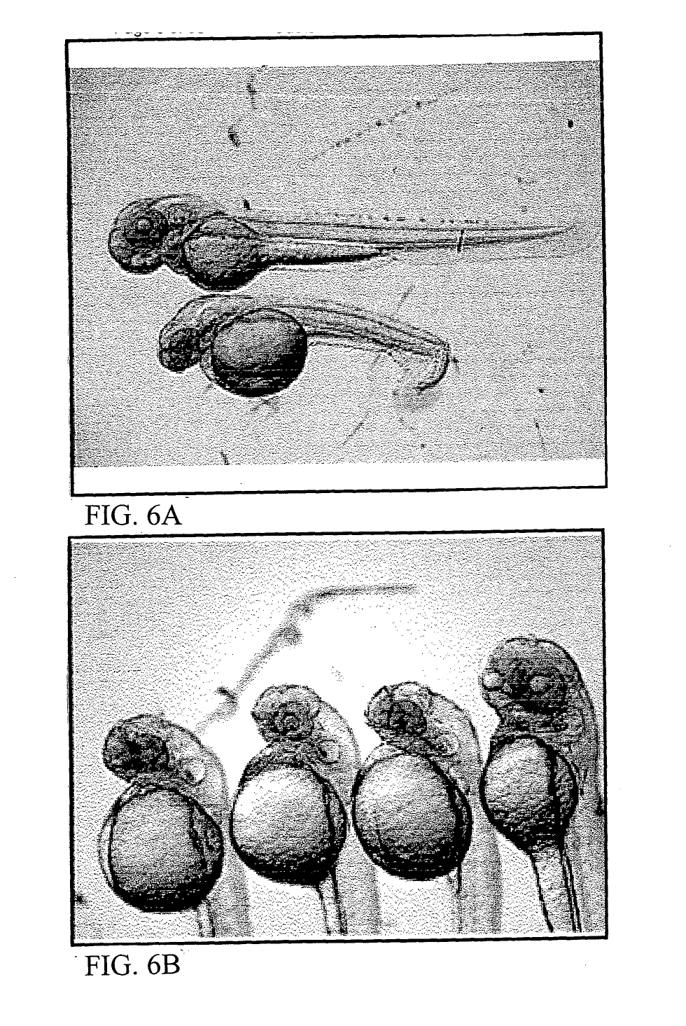

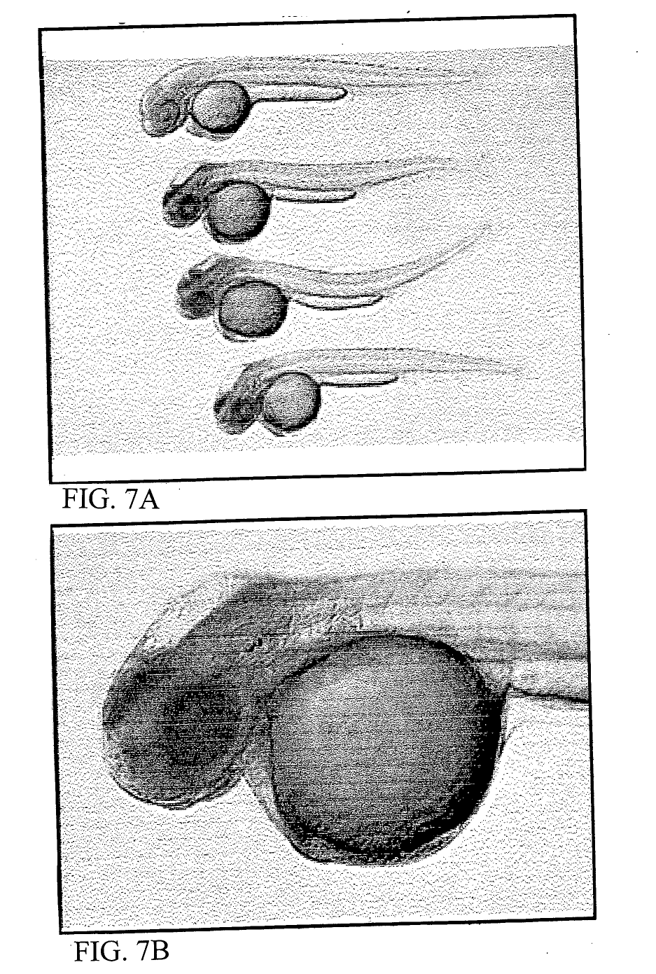

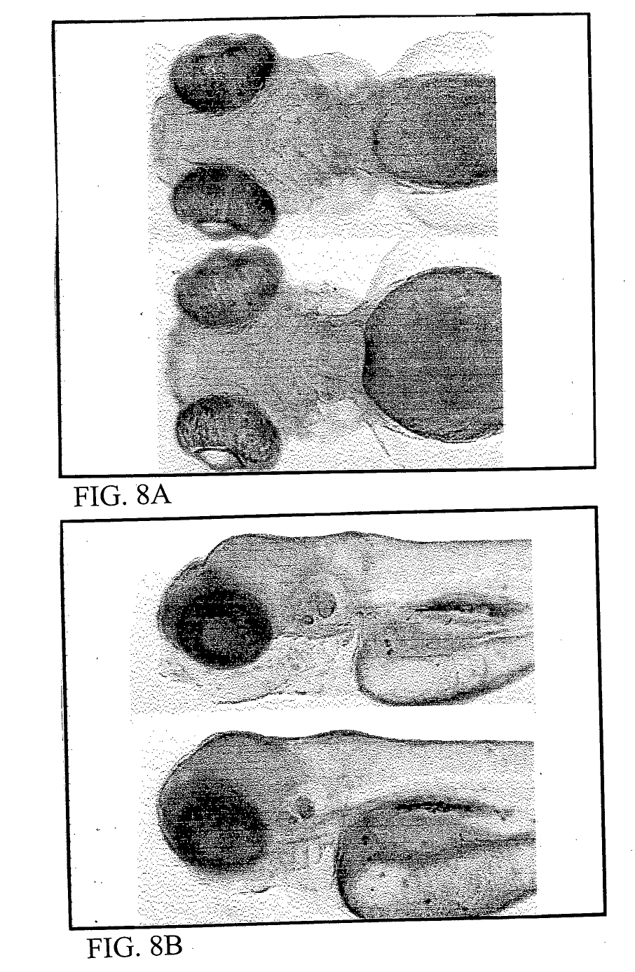

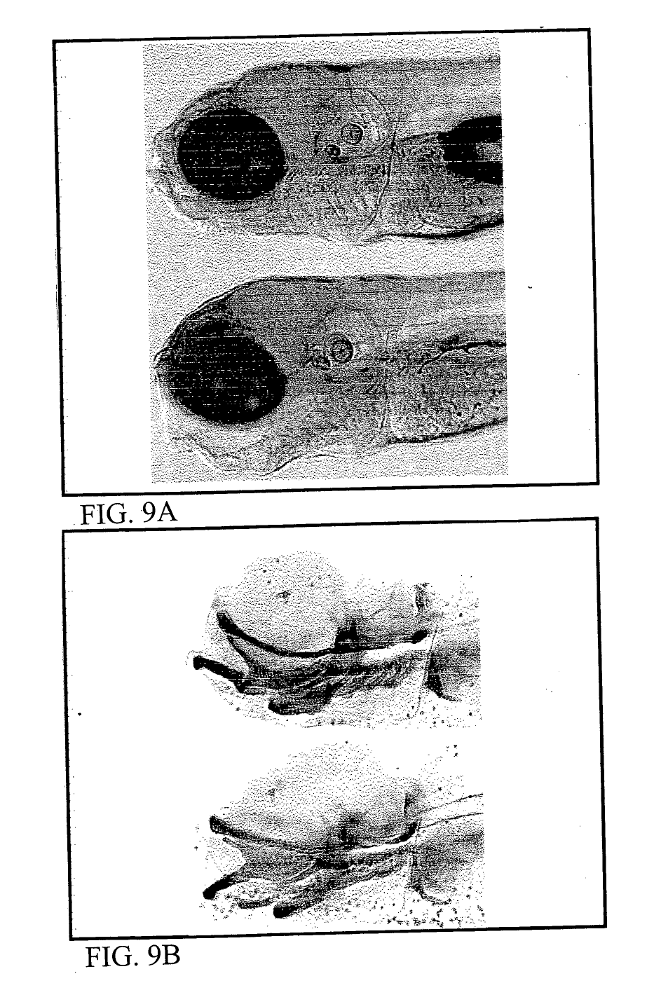

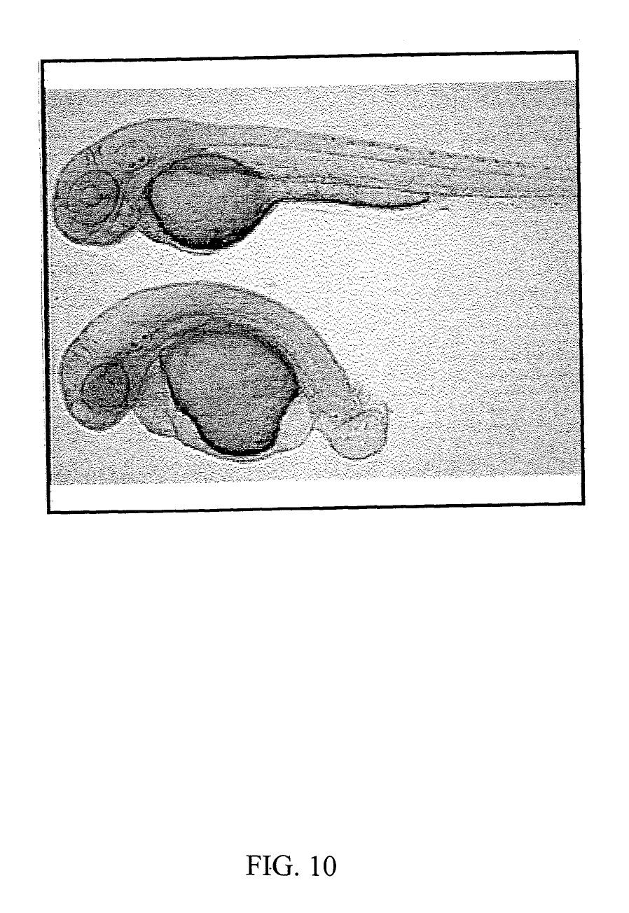

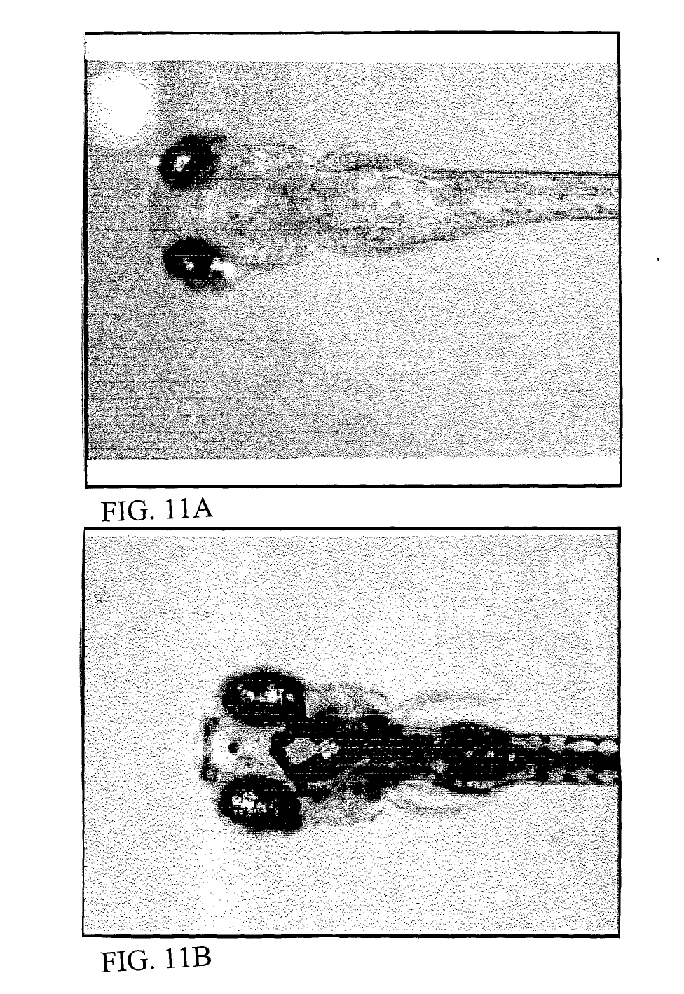

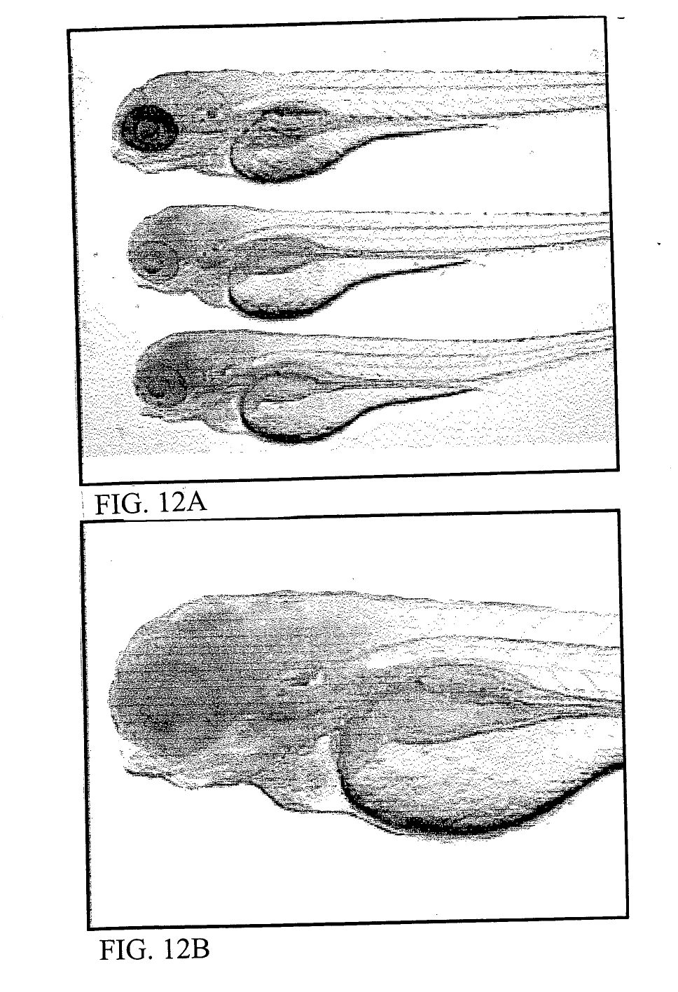

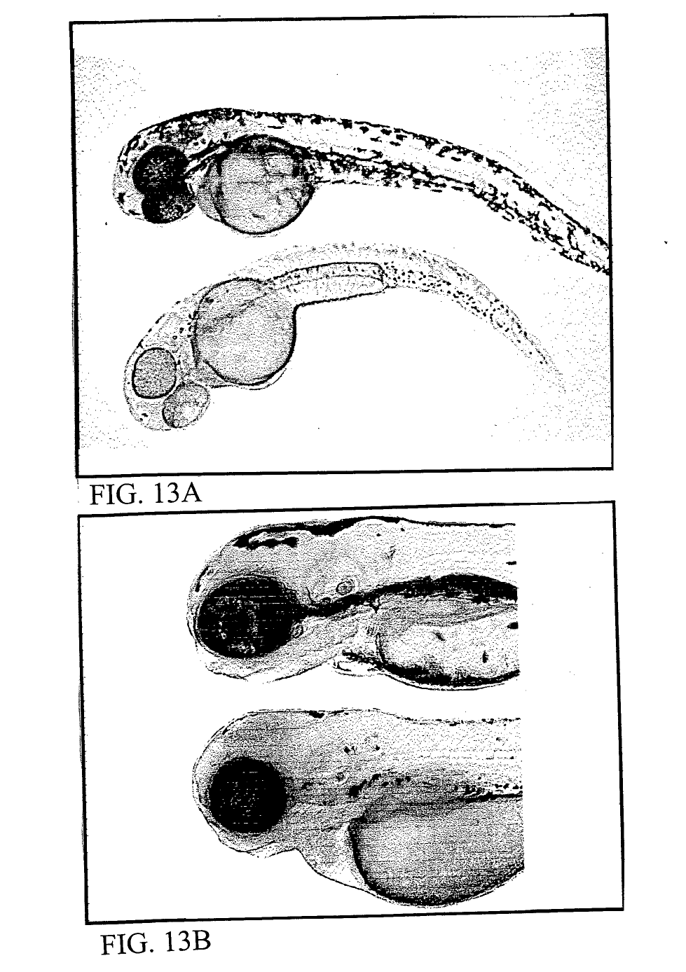

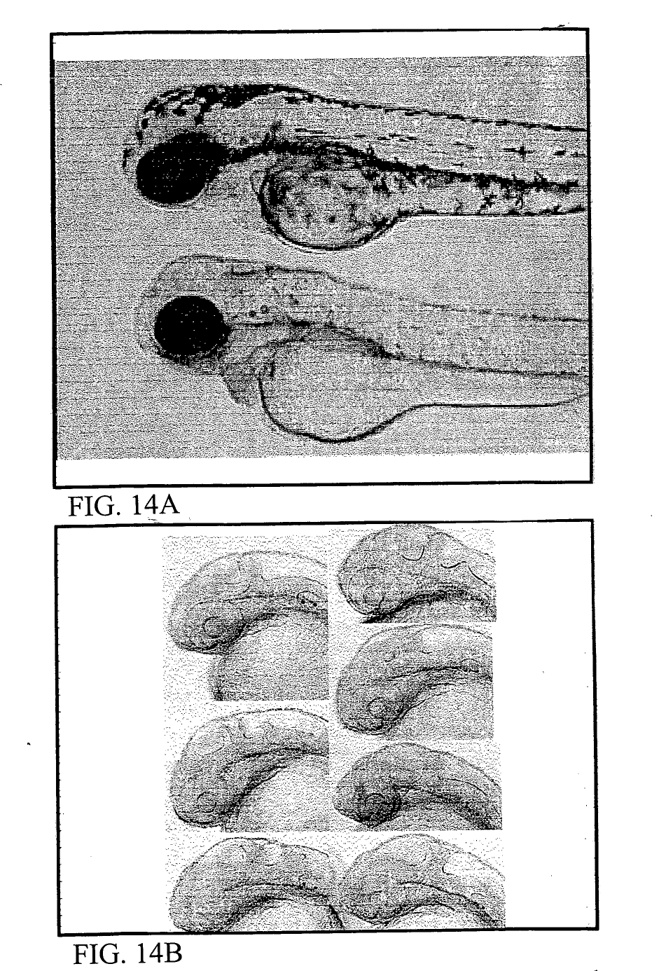

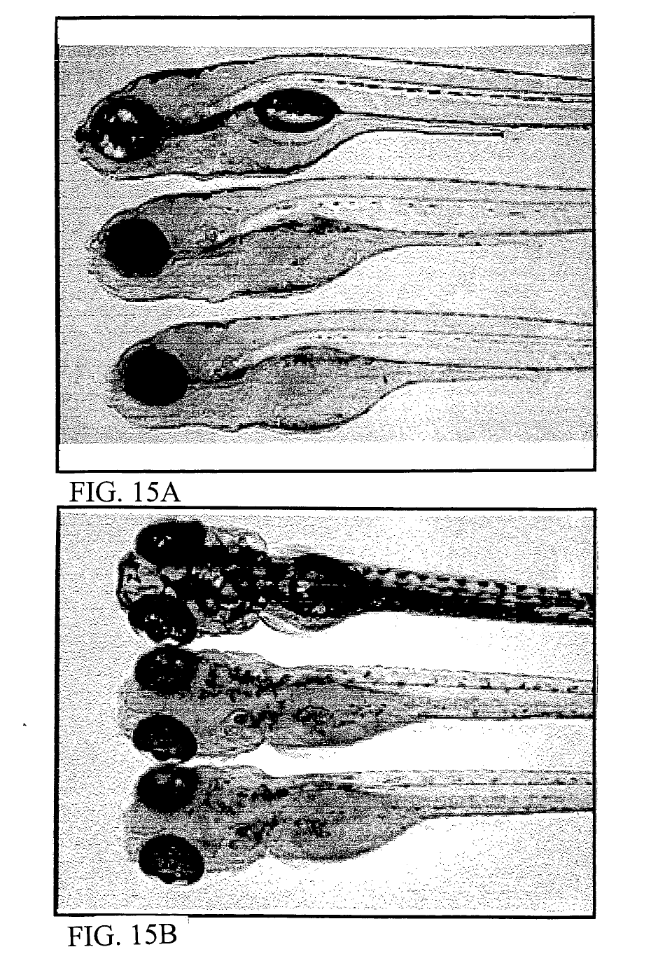

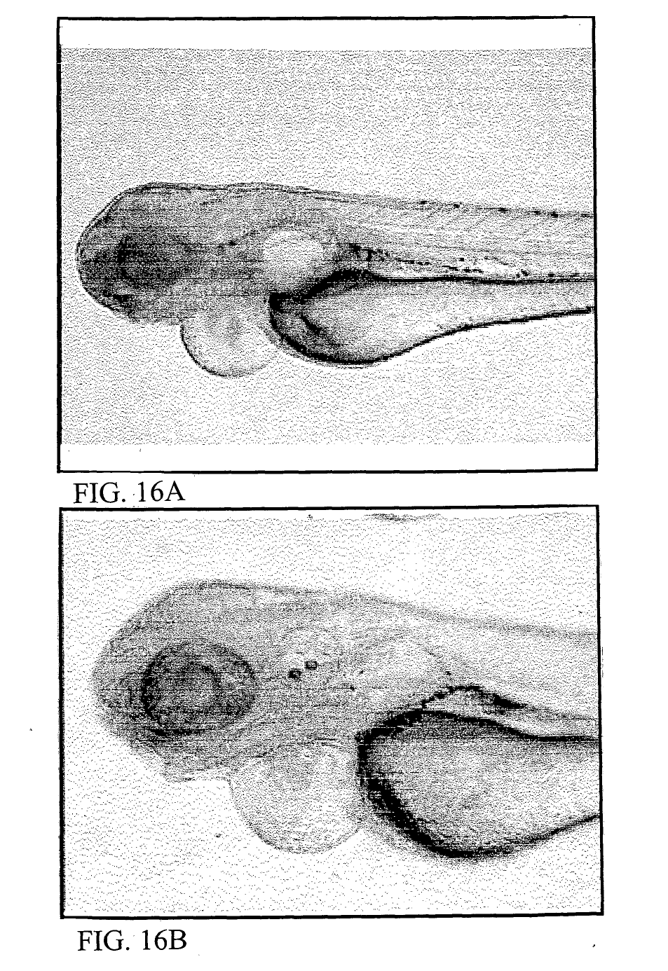

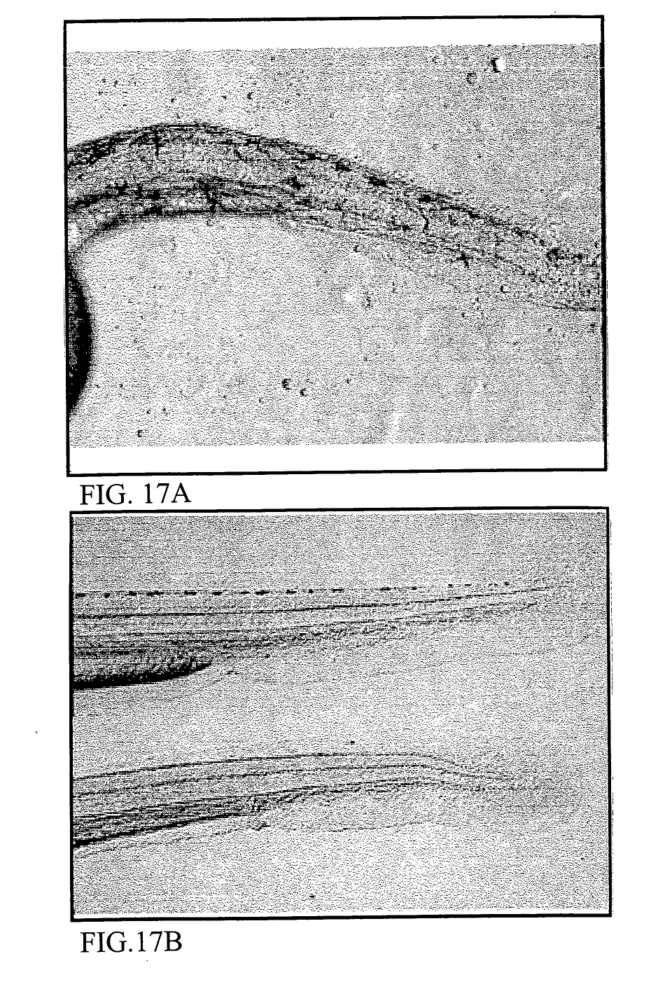

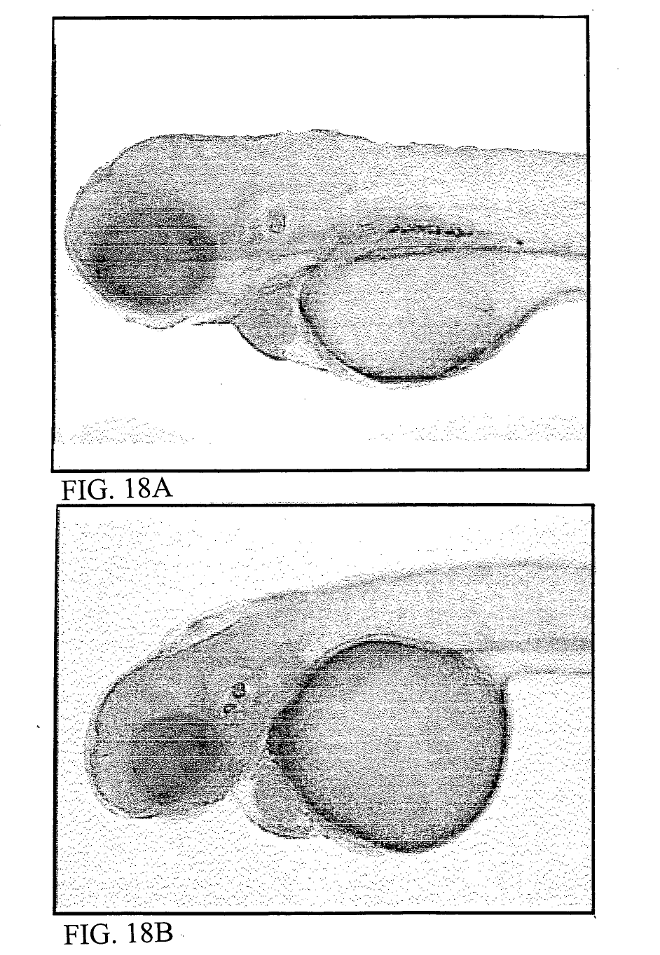

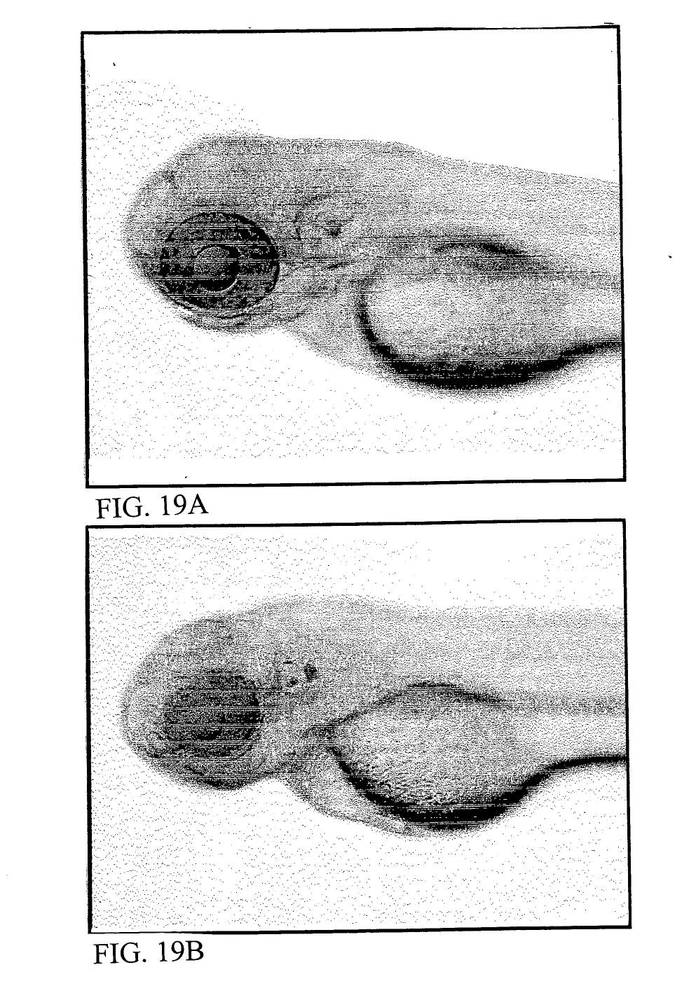

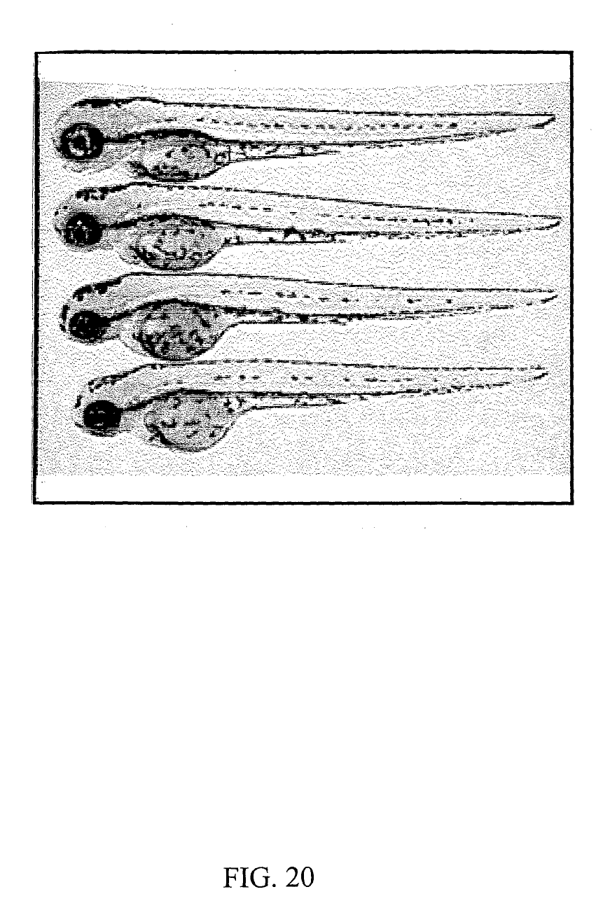

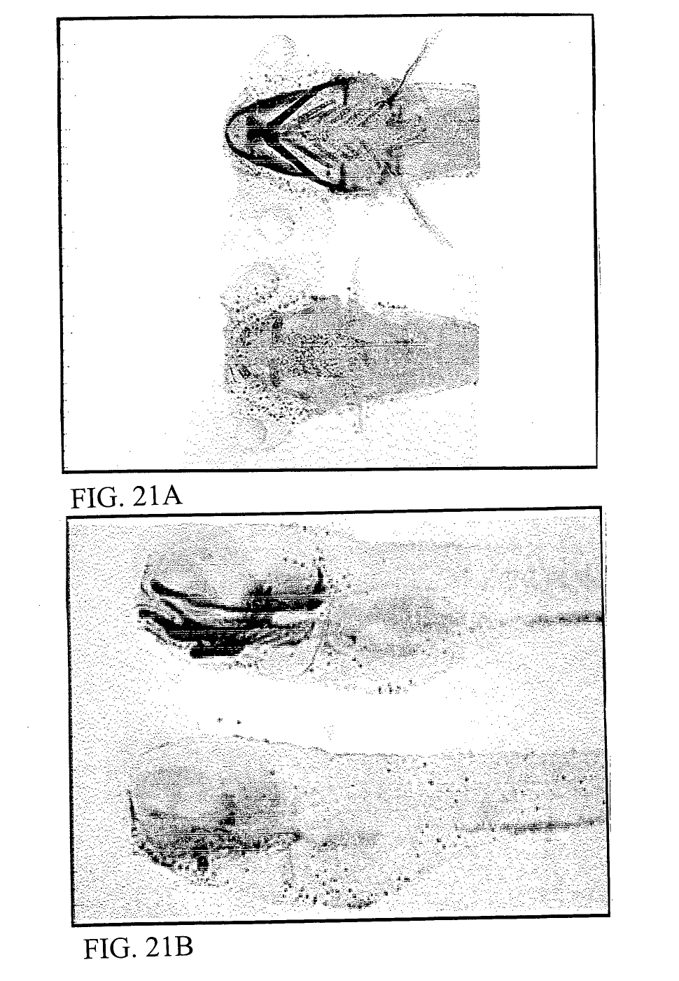

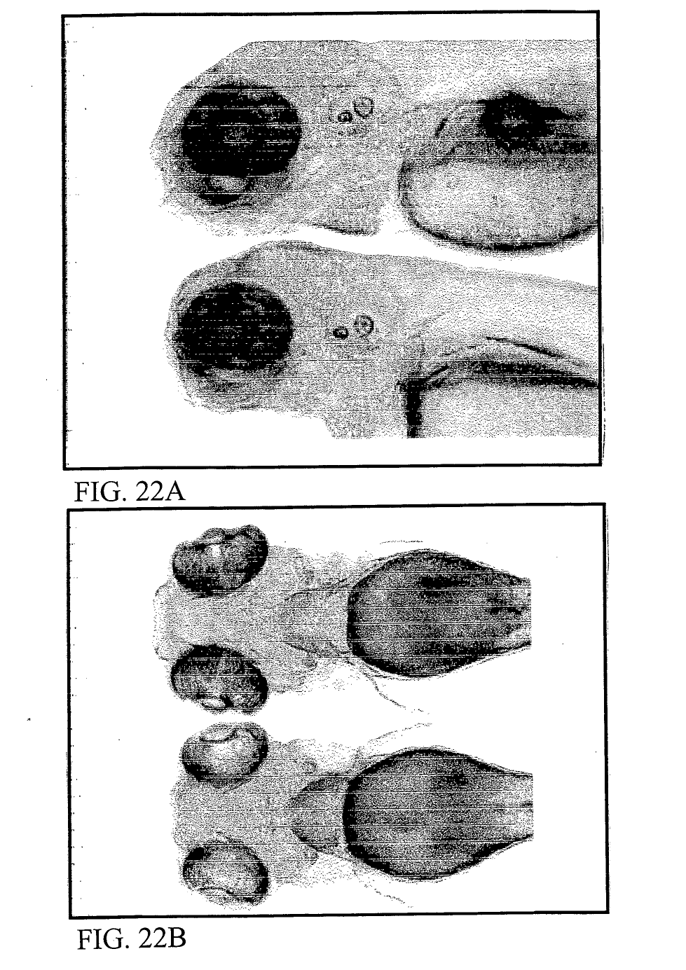

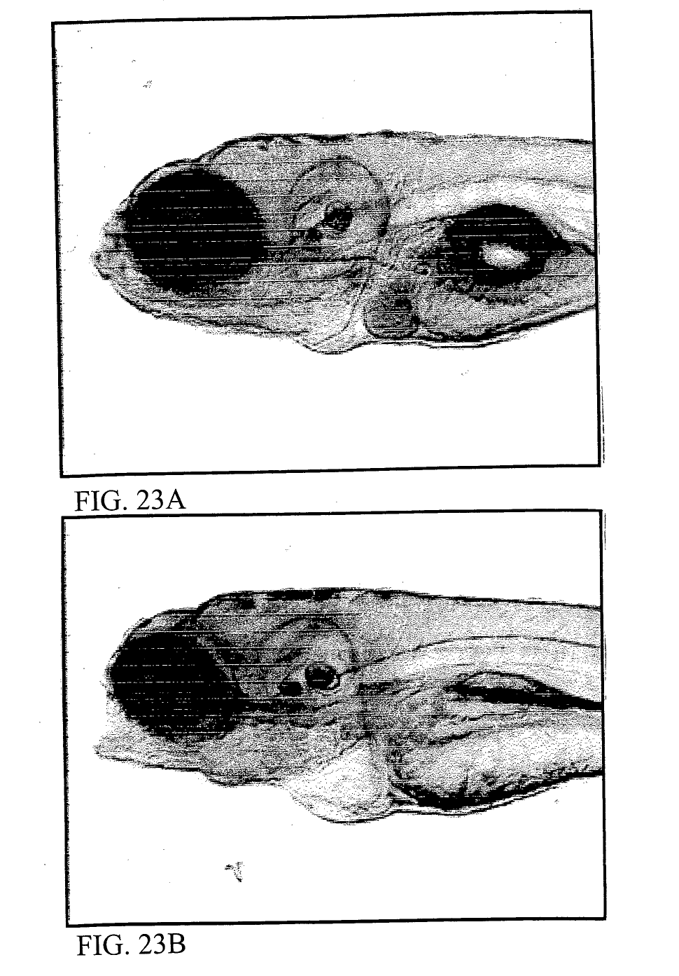

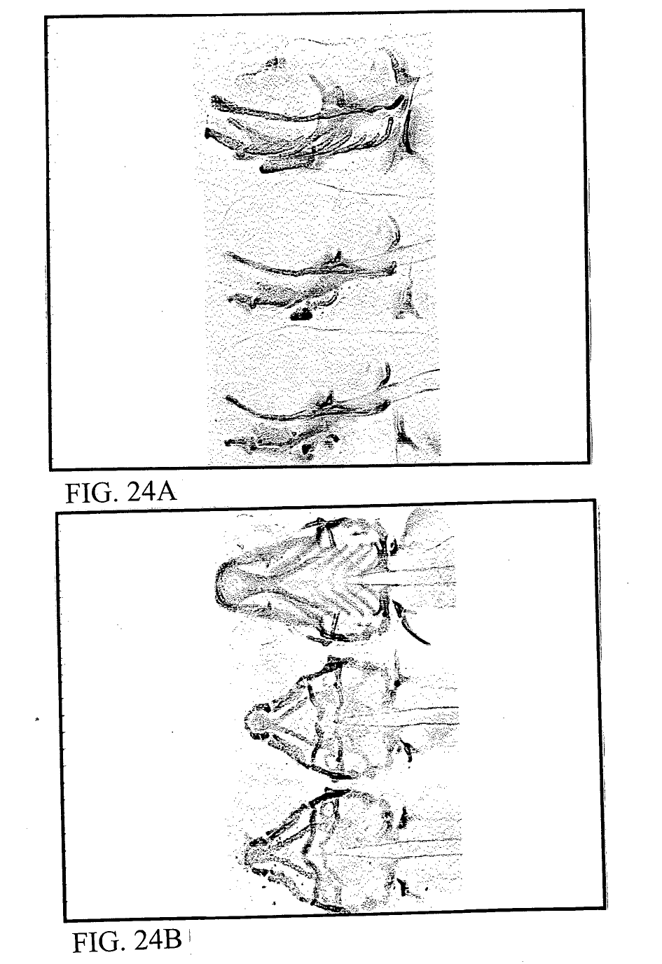

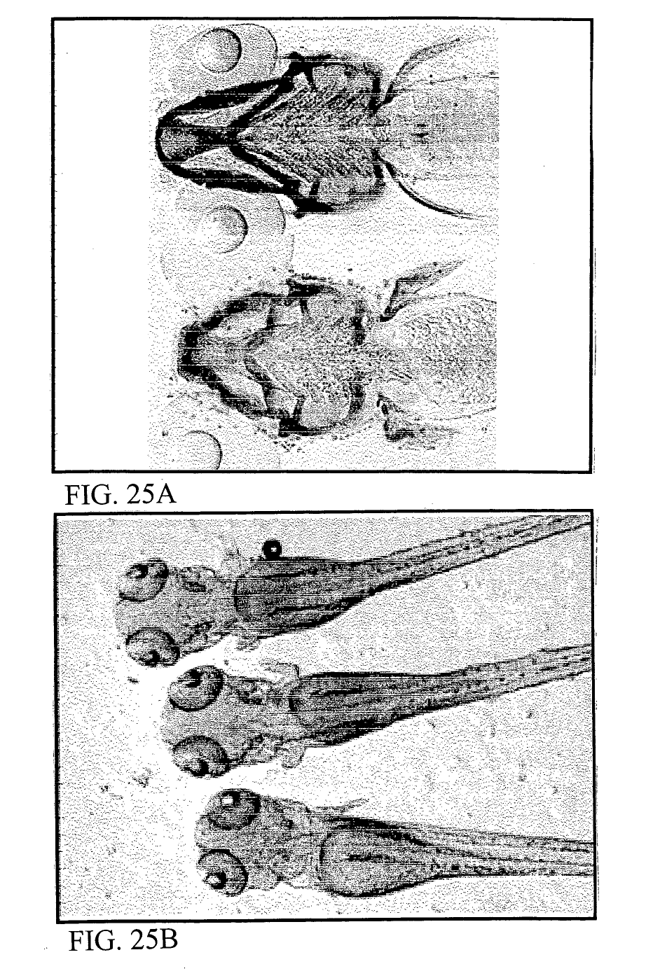

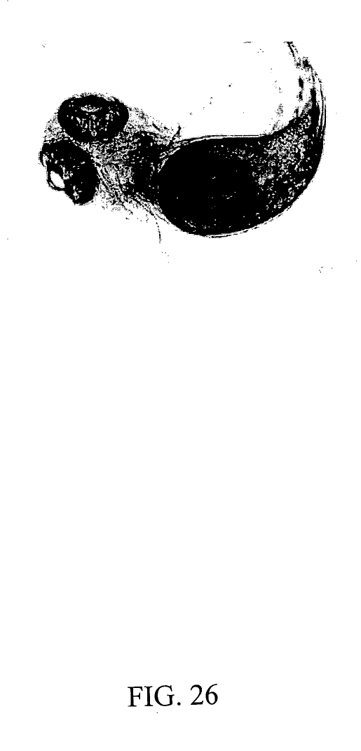

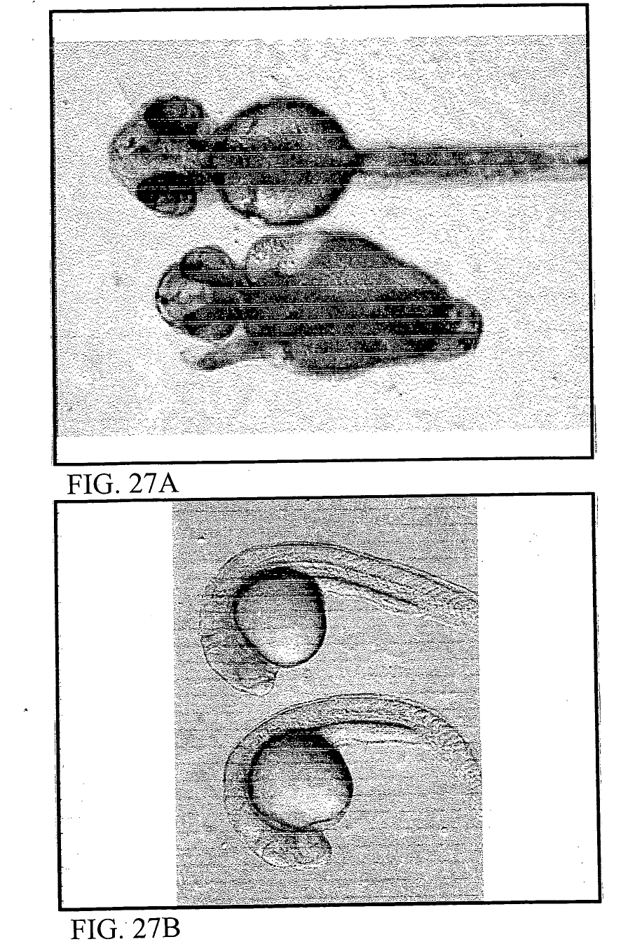

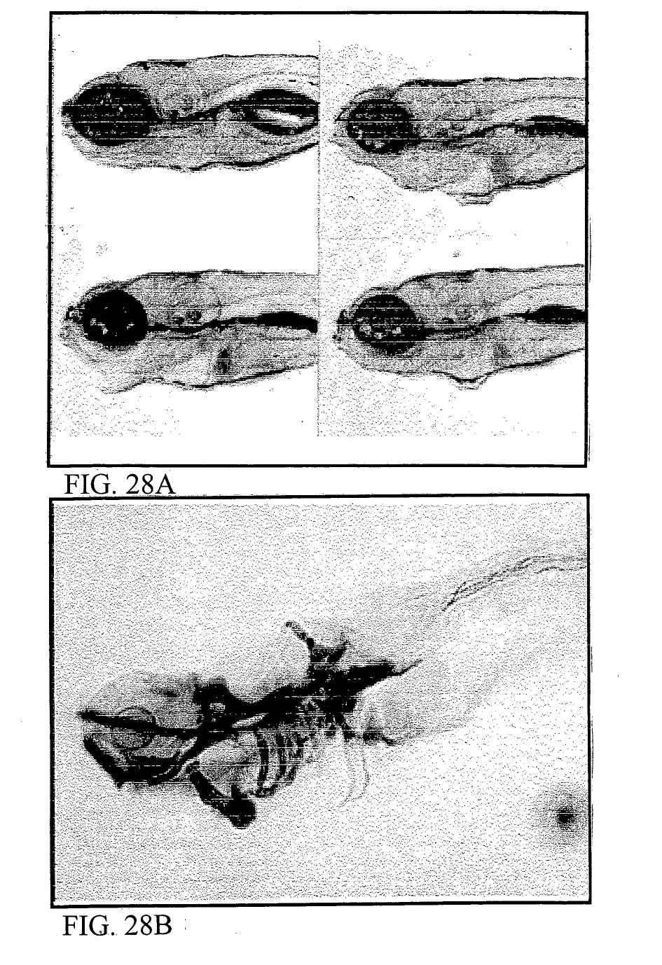

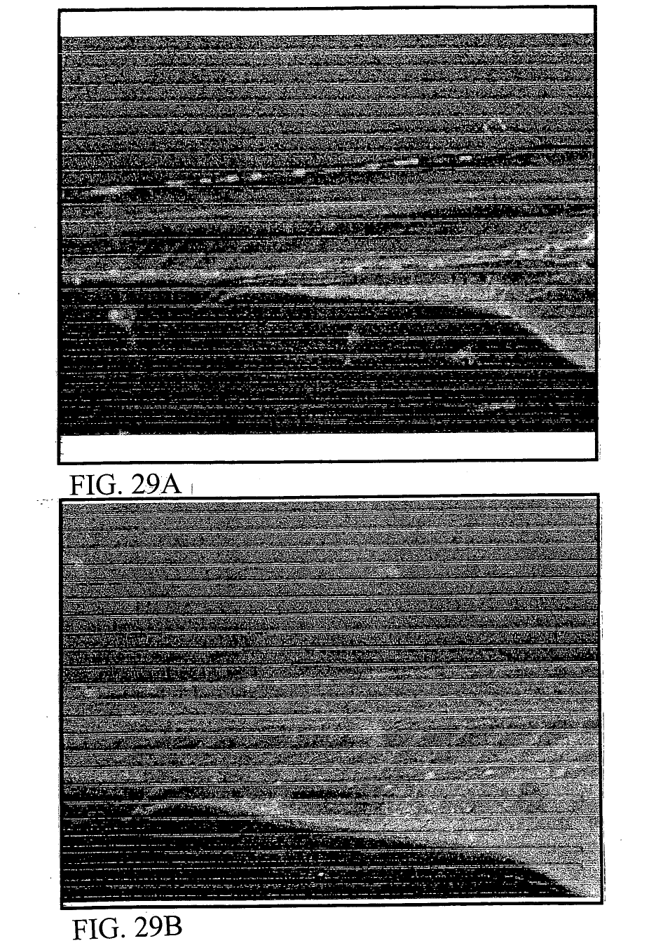

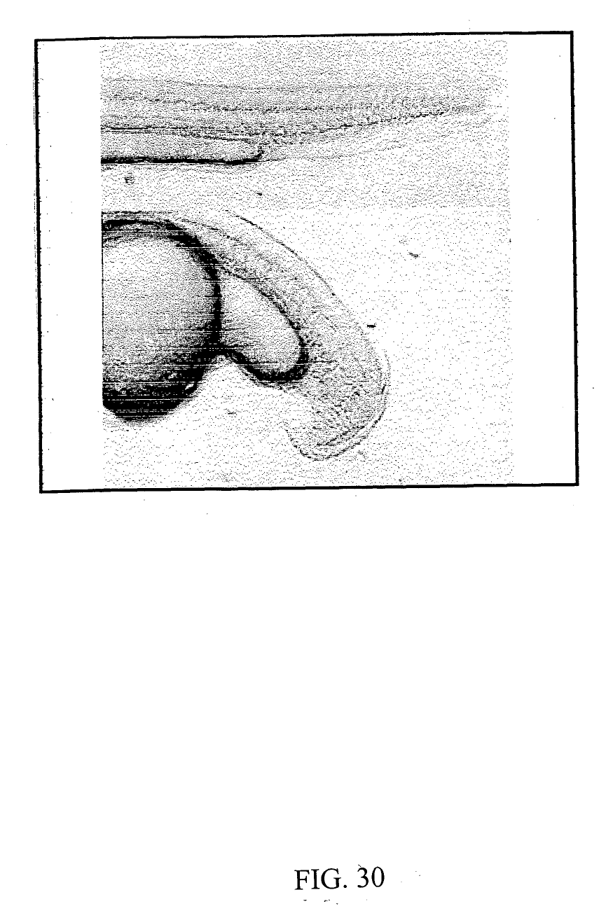

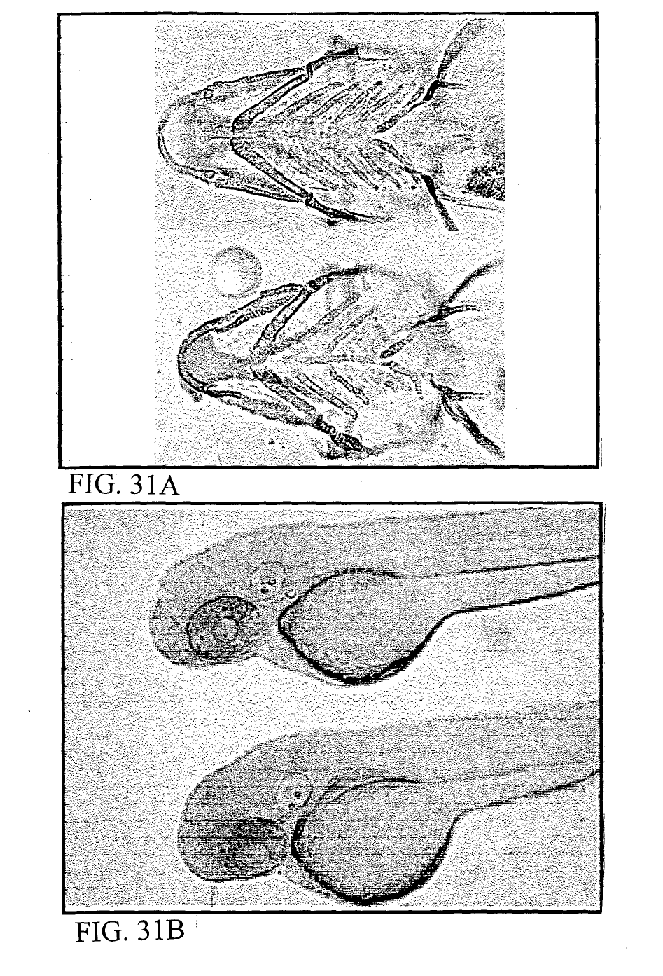

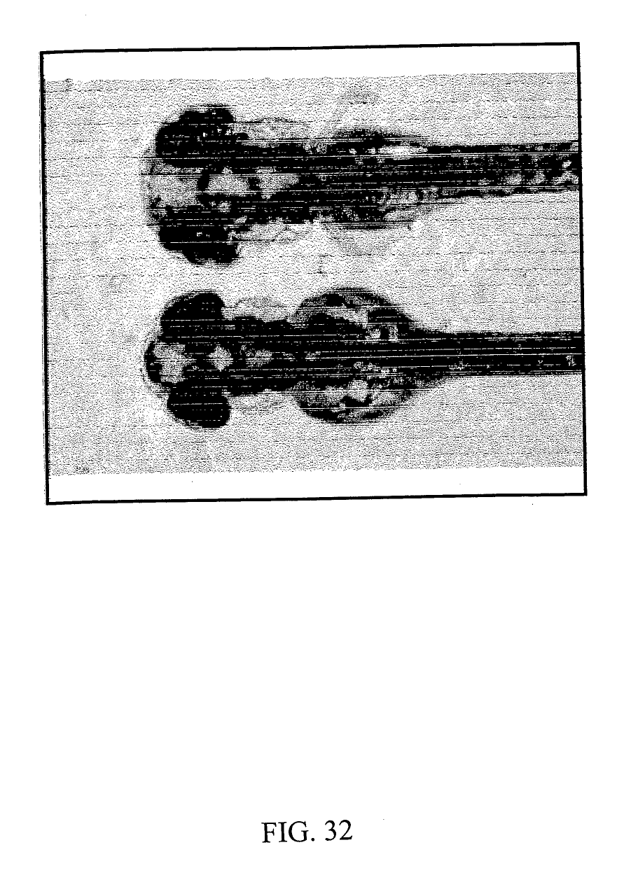

We identified the mutagenic inserts as follows. We performed Southern blots using DNA from individual embryos having the desired phenotype (the entire sample was used) and from tails of adults which mated (approximately 20% of the DNA sample was used). For the Southern blot, the DNA was digested using BglII and probed with Probe 1 (see FIG. 3). We then identified the band common to all embryos having the desired phenotype and which is uniquely homozygosed in pairs of fish which gave the phenotype. The size of the band was then established to estimate the distance to the 5′ genomic BglII site. We then reprobed the blot with Probe 2 (see FIG. 3) to determine the distance to the 3′ genomic BglII site. If the 3′ and 5′ genomic BglII sites were easily amplified by inverse PCR and there were few other inserts present whose amplification product is smaller than, or close to, the desired product in size, we performed inverse PCR using the appropriate primers (see FIG. 3) as follows: for the 5′ side, primer 1 plus primer 3 (BglII), or primer 5 (NcoI or BamH1); for the 3′ side, primer 7 plus primer 2 (PstI), primer 4 (BglII), or primer 6 (NcoI). We then isolated the appropriately sized PCR products.

If inverse PCR could not easily be used to amplify the 3′ and 5′ genomic BglII sites, we mapped additional potential inverse PCR sites using further Southern analysis on DNA obtained from several tails now established as carriers or non-carriers of the desired insertion. This DNA was digested with NcoI, NcoI plus BspHI, and BamHI to probe with Probe 1. In addition, the DNA was digested with NcoI, NcoI plus BspHI, PstI, and PstI plus NsiI to probe with Probe 2. While BspHI and NsiI do not cut in the provirus, their restriction products are compatible for ligation with NcoI- and PstI-cut DNA, respectively.

Furthermore, if the additional Southern analysis did not yield sites that can be used for inverse PCR, we cut the tails of additional F2 fish for more Southern analysis with the enzymes to be used for inverse PCR. However, if an appropriate sample still could not be found, we outcrossed the fish with the fewest additional inserts and raised the offspring to five days of age and lysed each individually in 96 well plates. We then performed Southern analysis with half of the sample to identify samples with only one or few enough other inserts to be used for inverse PCR. Inverse PCR was carried out as described in Allende et al. (Genes & Dev. 10:3141-3155, 1996).

After inverse PCR, the sequences of the bands were compared with the public database with BLAST (Altschul et al., J. Mol. Biol. 215:403-410, 1990), and when significant homologies were found (usually to zebrafish Expressed Sequence Tags (ESTs)), expression of those genes were analyzed in mutant and wild-type embryos by Northern analysis, RT-PCR, and/or in situ hybridization as described in Allende et al. (Genes & Dev. 10:3141-3155, 1996).

Generation of Zebrafish Mutants

1. Preparation of High Titer Stocks of F5 Virus

We used two viruses to generate founder fish, F5 and GT. A cell line producing the high titer F5 virus was prepared as follows. We obtained packaging cell line, 293 gp/bsr (Miyoshi et al., Proc. Natl. Acad. Sci. USA 94:10319-10323, 1997), infected it with a virus, SFGnlslacZ (Gaiano et al., Proc. Natl. Acad. Sci. USA 93:7777-7782, 1996), and selected a clone of cells designated F5 that yielded virus with high titer on both mouse 3T3 cells and a fish cell line PAC2 (Culp, Ph.D. Thesis, Massachusetts Institute of Technology, Cambridge, Mass. 1994), as determined by lacZ staining. Virus stocks were prepared by calcium phosphate transfection (Graham and van der Eb, Virology 52:456-467, 1973). During the course of the work, we found that lacZ titering of viruses on PAC2 cells was unreproducible, so we developed an assay to titer viruses on injected embryos.

2. Injecting Virus: Monitoring Successful Injections by the Embryo Assay

To assess the efficiency with which injected embryos are infected, we used either quantitative Southern blotting or quantitative PCR. Two to five days after every injection session, several injected embryos were lysed and their DNA extracted for analysis. Two genomic sequences were probed, a single-locus gene RAG2 (Willett et al., Immunogenetics 45:394-404, 1997) and proviral sequences. The ratio of these signals was normalized to signals from DNA of a fish heterozygous for a single insertion. The result, designated the embryo assay value, was used as a measure of the average number of proviral integrations per cell. Injected eggs that were raised were assumed to have the same embryo assay value as those that were sampled from the same injected batch.

To determine that the embryo assay was a good predictor of efficient germ-line transmission of proviral insertions, founder fish from batches of injected embryos with a range of embryo assay values were tested to determine the amount of provirus they could transmit to their F1. We outcrossed the founders and used the quantitative assay for RAG2 versus proviral sequences on DNA extracted from pools of their F1 progeny. Although there was considerable variation between founders from injections that had yielded the same embryo assay value, there was a definite correlation between embryo assay and average provirus transmission rate. Most founders from injections with embryo assays below 2 did not transmit well enough for our purposes, about half the founders with embryo assays of 2-5 transmitted sufficiently well, and nearly all founders with embryo assays over 5 transmitted well with an average of greater than one insert per gamete. At this transmission rate, we found that a substantial proportion of F1 fish inherit multiple proviral insertions. With F5 virus, we kept batches of embryos from injections in which the embryo assay values ranged from 2 to 11.4. Injections to make 36,000 founder fish, of which 15,000 were made with F5 virus, were performed 5 days/week for 11 months by one to two injectors per day. We estimate that 250,000 embryos were injected, and hence the overall survival of injected embryos to adulthood was approximately 15%.

3. Generating F1 Families, Selecting Multi-Insert F1 Fish, and Identifying Dominant Visible Mutations

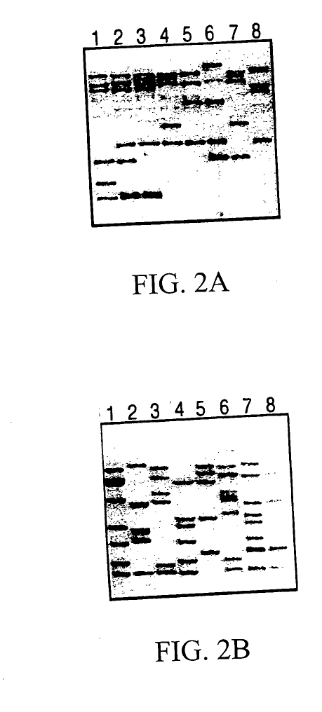

To generate F1 families, we mate founder fish to each other. There is considerable variation in the number of inserts between fish in a single F1 family, as well as between F1 families. To identify fish with the most non-overlapping inserts, 30 F1 fish from each cross are raised for 8 to 12 weeks and then their tail fins are clipped (Westerfield, The zebrafish book University of Oregon Press, Eugene, Oreg., 1995). The fish are held in individual cups while DNA is extracted from the fin clips. A small amount of DNA is analyzed by real time quantitative PCR (Heid et al., Genome Res. 6:986-994, 1996) to identify the eight fish with the greatest number of inserts in each family, and the rest of the sample from these eight fish is used for Southern blot analysis. As shown in FIG. 2, the F1 fish with the greatest number of inserts are often derived from the same germ cell(s) and hence share proviral insertions.

4. Generating F2families, Screening F3 Embryos, and Demonstrating that Mutants are Caused by Proviral Insertions

To generate F2 families, multi-insert F1 fish are mated and 50-70 embryos from each pair are raised. We perform sibling crosses of F2 fish at 3 months of age or older and examine their F3 embryos in a dissecting microscope to identify mutants. We examine embryos at 24 and 48 hr after fertilization and at 5 days of age (approximately 120 hr after fertilization). At day 5, embryos are screened for swimming behavior, then anesthetized, and visible structures are examined for defects.

To identify which of the insertions (of a total of approximately 10) segregating in an F2 family is linked to an identified mutation, Southern analysis is performed on DNA extracted from fin clips of parents of all the crosses screened in the family. A specific insert (Southern band) must be shared by both parents of every cross that showed the phenotype, and must be in either only one, or neither of the parents of all crosses that did not show the phenotype. We also perform Southern analysis on DNA from individual mutant embryos to look for the presence of this band. An unlinked band would only be present in three-fourths of the embryos, whereas a linked band must be in all of them. Often one can tell from the relative intensity of the bands that the candidate band is homozygous in mutant embryos. However, to obtain stronger evidence of tight linkage, we use a probe to genomic DNA flanking the candidate band.

Once a candidate band is identified, a junction fragment from either or both sides of this insertion is cloned by inverse PCR (Ochman et al., Genetics 120:621-623, 1988). The strategy used to clone the correct junction fragment from families with many inserts is shown in FIG. 3. The junction fragment is then used for two purposes: First, to distinguish between chromosomes with and without the putative mutagenic insertion on Southern blots, thereby determining whether mutant embryos are invariably homozygous for the mutagenic insert and verifying that their wild-type siblings never are; and second, to see if the junction fragment, when sequenced, has homology to a known gene or EST in the public database.

For recessive mutants, heterozygotes were crossed, embryos were sorted as having a mutant phenotypic or as being wild-type, and DNA was extracted from individual embryos and analyzed by Southern analysis for genotyping. For dominant mutants, heterozygotes were crossed to wild-type fish, juvenile fish were sorted by phenotype, and DNA was extracted from fin clips and analyzed by Southern analysis or PCR.

### Genes and Disease

The invention provides methods for the diagnosis and treatment of a variety of diseases or disorders, including human diseases and disorders. In addition, the invention also provides methods for the identification of therapeutic compounds for the treatment of these diseases or disorders.

Proliferative Disorders

Zebrafish with proliferative disorders, for example, those containing a mutation in a 904, Pescadillo, 1055-1, Valyl-tRNA Synthase, Translation Elongation Factor eEF1 Alpha, 821-02, Cyclin A2, Kinesin-Related Motor Protein EG5, 459, or 299 nucleic acid or amino acid sequence may be useful for the diagnosis, treatment, and/or identification of therapeutic compounds in cancer. One example of a zebrafish mutant with a proliferative disorder is the 904 mutant. 904 mutants display an overgrowth of neuronal tissues. Many cancers arise due to defects in the regulation of the cell cycle. Mutants, such as Pescadillo, 1055-1, or Cyclin A2, contain viral insertions in cell cycle checkpoint genes that are also of interest in the diagnosis and treatment of cancer or neuroblastoma. Alternatively, mutations that result in increased apoptosis may identify drug targets that are useful for the treatment of proliferative disorders, such potentially useful mutations may be in a Kinesin-Related Motor Protein EG5, 459, Valyl-tRNA Synthase, Translation Elongation Factor eEF1 Alpha, 821-02, or 299 nucleic acid sequence. In addition, 904, Pescadillo, 1055-1, Cyclin A2, Kinesin-Related Motor Protein EG5, 459, and 299 nucleic acid or amino acid sequences may be used in methods to diagnose, prevent, and treat proliferative disorders using the methods described herein.

Bone Connective Tissue, or Cartilage Formation Disorders

Zebrafish with defects in bone, connective tissue, or cartilage development, for example, those containing a mutation in a 954, Histone Deacetylase, 215, 307, 572, 1116A, 1548, Casein Kinase 1α, smoothened, 299, TCP-1 Beta, Non-Muscle Adenylosuccinate Synthase, Nuclear Cap Binding Protein Subunit 2, Ornithine Decarboxylase, 1447, and 994 nucleic acid or amino acid sequence may be useful for the diagnosis or treatment of a cartilage, connective tissue, or bone-related disorder. In particular, such potentially useful mutants include those with viral insertions in the 954 and Histone Deacetylase genes. Zebrafish containing a mutation in a 954 nucleic acid sequence lack dTDP-glucose 4-6-dehydratases, and have cartilage that fails to stain with Alcian blue. A histone deacetylase mutant also fails to stain with Alcian blue, and lacks many cartilaginous structure such as jaws, neurocranium, and fins. The 215 mutant has defects in jaw and ceratohyal formation. The 307, 572, 1116A, 1548, Casein Kinase 1α, Smoothened, 299 and 994 mutants have defects in the formation of cartilaginous structures. In addition, 954, histone deacetylase, 215, 307, 572, 1116A, 1548, Casein kinase 1α, smoothened, 299, TCP-1 Beta, Non-Muscle Adenylosuccinate Synthase, Nuclear Cap Binding Protein Subunit 2, Ornithine Decarboxylase, 1447, and 994 nucleic acid or amino acid sequences may be used in methods to diagnose, prevent, and treat defects in bone, connective tissue, or cartilage development using the methods described herein. Cartilage or connective tissue disorders that may be diagnosed or treated using the nucleotides or polypeptides described herein include, for example, arthritis, rheumatoid arthritis, juvenile rheumatoid arthritis, Osteogenesis Imperfecta, or Marfan syndrome. Bone diseases or disorders that may be diagnosed or treated using the nucleotides or polypeptides described herein include, for example, Paget's disease, fibrous dysplasia, osteochondritis dissecans, Saethre-Chotzen syndrome, or osteoporosis

Cell Death Disorders

Cell death, for example, apoptosis, plays a role in a number of neurodegenerative disorders. There is mounting evidence supporting an apoptosis-necrosis cell death continuum. In this continuum, neuronal death can result from varying contributions of coexisting apoptotic and necrotic mechanisms (Martin, Int. J Mol. Med. 7:455-78, 2001). A number of zebrafish mutants, such as U2AF, Ribonucleotide Reductase R1 Class 1, Kinesin-Related Motor Protein EG5, 459, 299, 1373, Denticleless, Ribonucleotide Reductase Protein R2, Telomeric Repeat Factor 2, SIL, U1 Small Nuclear Ribonucleoprotein C, Ski Interacting Protein, 297, Small Nuclear Ribonucleoprotein D1 Protein, or DNA Polymerase Epsilon Subunit B, 1045, Spliceosome Associated Protein 49, DNA Replication Licensing Factor MCM7, 1581, DEAD-Box RNA helicase (DEAD5 or DEAD19), cyclin A2, ISWI/SNF2, XCAP-C, DNA Replication Licensing Factor MCM2, DNA Replication Licensing Factor MCM3, Nuclear Cap Binding Protein Subunit 2, Ornithine Decarboxylase, ARS2 and Protein Phosphatase 1 Nuclear Targeting Subunit, Valyl-tRNA Synthase, Translation Elongation Factor eEF1 Alpha, Non-Muscle Adenylosuccinate Synthase, and 821-02 mutants display either necrotic or apoptotic cell death. Accordingly, these nucleic acid and amino acid sequences and zebrafish with defects in any of these nucleic acid or amino acid sequences may be used in the diagnosis, prevention, or treatment of cell death disorders using the methods described herein. Zebrafish mutants that show excess apoptotic cell death, such as Kinesin-Related Motor Protein EG5, 459, Valyl-tRNA Synthase, Translation Elongation Factor eEF1 Alpha, 821-02, and 299 are of particular interest. Cell death disorders that may be treated using the polypeptides and polynucleotides described herein include, for example, neurodegenerative disorders characterized by excess cell death, such as Alzheimer's disease, Parkinson's disease, spinocerebellar ataxia, or Huntington's disease. Necrotic disorders that may be treated using the polypeptides and polynucleotides described herein include, for example, Leigh's disease or subacute necrotizing encephalomyelopathy

Circulatory Defects

Multiple cell populations are involved in the morphogenesis of the cardiac, arterial, and venous systems as well as in the correct alignment and connection of the developing vessels within the cardiac chambers. The development of this intricate cell network is subject to a high rate of congenital abnormalities. Zebrafish with defects in the formation of the circulatory system, for example, those containing a mutation in a 904, 429, 1463, or Mitochondrial Inner Membrane Translocating (rTIM23) nucleic acid or amino acid sequence may be useful in the diagnosis or treatment of diseases or disorders of the circulatory system. In addition, 904, 429, 1463, and Mitochondrial Inner Membrane Translocating (rTIM23) nucleic acid and amino acid sequences may be used to diagnose, treat, or prevent circulatory disorders using the methods described herein. Circulatory, disorders that may be treated using the polypeptides and polynucleotides described herein include, for example, atherosclerosis, stroke, thrombosis, peripheral arterial disease, hypertension, hypotension, or peripheral vascular disease.

Craniofacial Defects

Molecular genetic studies have shown that mutations in the genes governing bone morphogenesis signaling networks cause a variety of human craniofacial defects. Such birth defects may be prevented or diagnosed if their etiology is understood. Zebrafish mutants with viral insertions in the following genes may be useful in the diagnosis, treatment, or prevention of such craniofacial birth defects: Nrp-1, 215, 307, 572, 1116A, 1548, Casein Kinase 1 α, Smoothened, 299, 297, DNA Replication Licensing Factor MCM2, 1257, TCP-1 Alpha, TCP-1 Beta, Nuclear Cap Binding Protein Subunit 2, Non-Muscle Adenylosuccinate Synthase, Ornithine Decarboxylase, and Sec61 Alpha. In addition, Nrp-1, 215, 307, 572, 1116A, 1548, casein kinase 1 α, Smoothened, 299, 297, DNA Replication Licensing Factor MCM2, 272, 1257, TCP-1 Alpha, TCP-1 Beta, Nuclear Cap Binding Protein Subunit 2, Non-Muscle Adenylosuccinate Synthase, Ornithine Decarboxylase, and Sec61 Alpha nucleic acid and amino acid molecules may also be used to diagnose, treat, or prevent craniofacial malformations using the methods described herein. Craniofacial disorders that may be treated, diagnosed, or prevented using the polypeptides and polynucleotides described herein include, for example, Apert, Crouzon, Pfeiffer and Saethre-Chotzen Syndromes, Treacher-Collins syndrome, Marfan's syndrome, or Angelman's disease, or an eye malformation syndrome, such as oculorenal syndrome, or Reiger syndrome.

Hearing Disorders

It is estimated that at least 1-1.5/1000 live births result in congenital permanent hearing impairment. Congenital hearing loss has profound effects on the speech, language, and social development of children. Many adults also suffer from varying degrees of aquired hearing loss. Mutants with viral insertions in the following genes have defects in otolith, semi-circular canal, or hair cell development: POU2, HNF1-β/vHNF1, U1 Small Nuclear Ribonucleoprotein C, Cyclin A2, ARS2, TCP-1 Eta, or Histone Deaceytlase. Accordingly, zebrafish mutant for a POU2, HNF1-β/vHNF1, U1 Small Nuclear Ribonucleoprotein C, Cyclin A2, ARS2, TCP-1 Eta or Histone Deaceytlase nucleic acid or amino acid sequence, as well as these nucleic acid and amino acid molecules themselves, may be useful for the diagnosis, prevention, or treatment of either congenital or acquired hearing disorders using the methods described herein. Disorders that may be treated, diagnosed, or prevented using the polypeptides and polynucleotides described herein include, for example, congenital deafness or a sensory disorder, such as Usher syndrome or Waardenburg syndrome, or acquired hearing loss resulting from Paget's disease or osteogenesis imperfecta.

Diabetes

Diabetes is a growing problem throughout the world. Identifying the underlying genetic defects that lead to diabetes may be useful for the prevention, diagnosis and/or treatment of this disease. Genes such as HNF1-β/vHNF1, 429, and 1447 are required for normal pancreas development in zebrafish. HNF1-β/vHNF1 mutants display an abnormal pancreas. Interestingly, HNF1-β/vHNF1 is homologous to human Hepatic Transcription Factor 1; mutations in human HNF1 cause a genetic form of human diabetes, MODY V (maturity onset diabetes of the young), in which patients have kidney defects in addition to diabetes (Iwasaki et al., Diabetes Care 21:2144-2148, 1998; Horikawa et al., Nat. Genet. 17:384-385, 1997). Accordingly, zebrafish containing a mutant HNF1-β/vHNF1, 429, or 1447 nucleic acid or amino acid sequence may be useful for identifying compounds that may be used to diagnose, treat, or prevent diabetes in humans. In addition, the HNF1-β/vHNF1, 429, and 1447 nucleic acid and amino acid sequences themselves may be used in diagnosing, preventing, or treating diabetes using the methods described herein. Pancreatic disorders that may be treated, diagnosed, or prevented using the polypeptides and polynucleotides described herein include, for example, juvenile diabetes, type I diabetes, type II diabetes, diabetes insipidus, or gestational diabetes.

Heart Defects and Disorders

Congenital heart defects are one of the most common form of birth defects. Nearly 1/125-1/150 children born each year have a congenital heart defect. Moreover, congenital heart defects are one of the most common causes of infant mortality. Identifying the underlying genetic defects that result in congenital heart defects may be useful for the prevention, diagnosis and/or treatment of congenital heart defects. Zebrafish with viral insertions in the 1548, Mitochondrial Inner Membrane Translocating Protein (rTIM23), or TCP-1 Alpha, histone deaceytlase, and Nuclear Cap Binding Protein Subunit 2 nucleic acid or amino acid sequences display heart defects. Accordingly, such zebrafish may be used to identify test compounds that may be used to diagnose, prevent, or treat human heart defects. In addition, 1548, Mitochondrial Inner Membrane Translocating Protein (rTIM23), or TCP-1 Alpha, histone deaceytlase, and Nuclear Cap Binding Protein Subunit 2 nucleic acid and amino acid molecules themselves may be useful in diagnosing, preventing, or treating human heart defects using the methods described herein. Heart defects, diseases, or disorders that may be treated using the polypeptides and polynucleotides described herein include, for example, congenital heart defects, such as ventricular or atrial septal defects, or heart disease, such as heart attack, congestive heart failure, or coronary artery disease.

Infertility

Various genetic defects can lead to infertility. Early embryonic death can be caused by genetic defects including defects in the spinster nucleic acid sequence. Accordingly, zebrafish mutant for a spinster nucleic acid or amino acid sequence may be used to identify test compounds that are useful in the diagnosis, prevention, or treatment of human infertility. In addition, spinster nucleic acid or amino acid molecules themselves may be used to diagnose, prevent, or treat human infertility using the methods described herein.

Kidney Disorders

Congenital kidney defects are a common birth defect. In fact, in 2002, more than 8 million Americans had seriously reduced kidney function and about 400,000 required dialysis or a kidney transplant to stay alive. This figure has doubled in the last ten years. One common form of kidney disorders involves cystic kidney disorders which are prevalent among children born with developmental defects in kidney formation. Cystic kidney disorders are also prevalent in adults. Cystic kidney disorders involve the formation of fluid-filled sacs in the kidney. These cysts tend to develop in weak segments of the tubules that carry urine from the glomeruli. The cyst's growth displaces healthy tissue. In polycystic kidney disease, cysts generally occur in both the left and the right kidney. The polycystic kidney often retains its shape despite the presence of multiple cysts. These cysts, however, may impair the kidney's function. In contrast, multicystic kidney disease usually affects only one of the two kidneys. The affected kidney loses its characteristic bean shape and, instead, resembles a cluster of grapes. Moreover, the affected kidney does not function.

Zebrafish with viral insertions in a 459 or HNF1-β/vHNF1 nucleic acid sequence may have kidney abnormalities, including, for example, a cystic kidney phenotype. Accordingly, zebrafish having a mutation in a 459 or HNF1-β/vHNF1 nucleic acid or amino acid sequence may be used in screen to identify compounds that are useful in diagnosing, preventing, or treating kidney disease. In addition, 459 or HNF1-β/vHNF1 nucleic acid and amino acid sequences themselves may be using in methods of diagnosing, preventing, or treating kidney disease, as described herein. Kidney defects, diseases, or disorders that may be treated using the polypeptides and polynucleotides described herein include, for example, polycystic kidney disease, multicystic kidney disease, malformation of the kidney, Bardet-Biedl syndrome, kidney failure, acute renal failure, nephrolithiasis, congenital nephritic syndrome, kidney infection, or kidney stones.

Limb Formation Defects

Defects in limb formation are another common birth defect. Zebrafish with viral insertions in genes such as Casein Kinase 1 α, Smoothened, and Histone Deacetylase have fin abnormalities that may be analogous to human limb formation abnormalities. Accordingly, zebrafish having a mutation in a Casein Kinase 1 α, Smoothened, and Histone Deacetylase nucleic acid or amino acid sequence may be used to identify test compounds that affect limb formation. In addition, Casein Kinase 1 α, Smoothened, and Histone Deacetylase nucleic acid and amino acid sequences themselves may be used in methods for diagnosing, preventing, or treating limb abnormalities. Limb formation defects, diseases, or disorders that may be treated using the polypeptides and polynucleotides described herein include, for example, such as achondroplasia, Ellis-van Creveld syndrome, Poland sequence, or Pfeiffer syndrome.

Mental Retardation and Mental Diseases

Zebrafish with defects in brain development may be used in methods of identifying test compounds that affect brain development. Accordingly, these mutant zebrafish, for example, zebrafish having a mutation in a Telomeric Repeat Factor 2, U1 Small Nuclear Ribonucleoprotein C, DNA polymerase Epsilon Subunit B, 1045, Spliceosome Associated Protein 49, DNA Replication Licensing Factor MCM7, DEAD-Box RNA Helicase (DEAD5 or DEAD19), Chromosomal Assembly Protein (XCAP-C), DNA Replication Licensing Factor MCM2, ARS2, 40S ribosomal protein S18, Histone Deacetylase, Protein Phosphatase 1 Nuclear Targeting Subunit protein (PNUTS), Ornithine Decarboxylase, 1447, U2AF, 1463, 1262, VPSP18, 994, 60S ribosomal L35, 60S ribosomal L44, Smoothened, SIL, Ski Interacting Protein, 297, Small Nuclear Ribonucleoprotein D1, 1581, Cyclin A2, ISWI/SNF, DNA Replication Licensing Factor MCM3, Valyl-tRNA Synthase, 40S ribosomal S5, TCP-1 Alpha, TCP-1 Beta, Translation Elongation Factor eEF1 Alpha, 1257, 60S Ribosomal Protein L24, Non-Muscle Adenylosuccinate Synthase, Nuclear Cap Binding Protein Subunit 2 Polypeptide, Sec61 Alpha, BAF53a, or TAFII-55 nucleic acid or amino acid sequence may be useful in diagnosing, preventing, or treating mental retardation. In addition, Telomeric Repeat Factor 2, U1 Small Nuclear Ribonucleoprotein C, DNA polymerase Epsilon Subunit B. 1045, Spliceosome Associated Protein 49, DNA Replication Licensing Factor MCM7, DEAD-Box RNA Helicase (DEAD5 or DEAD19), Chromosomal Assembly Protein (XCAP-C), DNA Replication Licensing Factor MCM2, ARS2, 40S ribosomal protein S18, Histone Deacetylase, Protein Phosphatase 1 Nuclear Targeting Subunit protein (PNUTS), Ornithine Decarboxylase, 1447, U2AF, 1463, 1262, VPSP18, 994, 60S ribosomal L35, 60S ribosomal L44, Smoothened, SIL, Ski Interacting Protein, 297, Small Nuclear Ribonucleoprotein D1, 1581, Cyclin A2, ISWI/SNF, DNA Replication Licensing Factor MCM3, Valyl-tRNA Synthase, 40S ribosomal S5, TCP-1 Alpha, TCP-1 Beta, Translation Elongation Factor eEF1 Alpha, 1257, 60S Ribosomal Protein L24, Non-Muscle Adenylosuccinate Synthase, Nuclear Cap Binding Protein Subunit 2 Polypeptide, Sec61 Alpha, BAF53a, or TAFII-55 nucleic acid and amino acid sequences themselves may be used in methods of diagnosing, preventing, and treating mental retardation. Mental defects, diseases, or disorders that may be treated using the polypeptides and polynucleotides described herein include, for example, Down's syndrome, Chiari Malformation, colpocephaly, holoprosencephaly, lissencephaly, or megalencephaly, microencephaly, anencephaly, autism, schizophrenia, depression, dementia, or bipolar disorders.

Muscle Defects

Defects in muscle development or function are another prevalent class of congenital birth defects. We determined that the zebrafish genes 428, Smoothened, Glypican-6 or Knypek, and Denticleless are required for proper muscle development. Accordingly, zebrafish mutant for a 428, Smoothened, Glypican-6 or Knypek, or Denticleless nucleic acid or amino acid sequence may be used in screens for idenifying compounds that affect muscle development. In addition, 428, Smoothened, Glypican-6 or Knypek, and Denticleless nucleic acid and amino acid sequence themselves may be used in methods for diagnosing, preventing, or treating congenital muscle disorders. Muscle defects, diseases, or disorders that may be treated using the polypeptides and polynucleotides described herein include, for example, myotonia congenital, inclusion body myositis, or a congenital muscular dystrophy.

Neurodegenerative Disorders

As people are living longer in developed countries, a quiet epidemic of dementia is emerging. By age 85, one of every three people is demented and the vast majority of these people suffer from neurodegenerative illnesses. In the United States alone, it is estimated that nearly 5 million people suffer from dementia. Understanding the causes of neurodegenerative disorders is extremely important to developing effective therapies. Zebrafish having a mutation in a Aryl Hydrocarbon Receptor Nuclear Translocator 2A, Vesicular Integral-Membrane Protein VIP 36, 297, 40S Ribosomal Protein S5, Cad-1, V-ATPase16 kDa Proteolytic Subunit, 459, 299, Ribonucleotide Reductase Protein R1 Class 1, Kinesin Related Motor Protein EG5, 821-02, Valyl-tRNA Synthase, or Translation Elongation Factor eEFI Alpha nucleic acid or amino acid sequence display neurodegenerative phenoypes. For example, many human patients with motor neuron disease are touch insensitive in addition to displaying impaired movement. The Cad-1 mutants are an example of a zebrafish mutant that displays a similar phenotype. These mutants are touch insensitive and lose motility. Accordingly, these zebrafish may be useful in screens to identify test compounds that may be used in diagnosing, preventing, or treating a neurodegenerative disorder. In addition, Aryl Hydrocarbon Receptor Nuclear Translocator 2A, Vesicular Integral-Membrane Protein VIP 36, 297, 40S Ribosomal Protein S5, Cad-1, V-ATPase 16 kDa Proteolytic Subunit, 459, 299, Ribonucleotide Reductase Protein R1 Class 1, Kinesin Related Motor Protein EG5, 821-02, Valyl-tRNA Synthase, or Translation Elongation Factor eEF1 Alpha nucleic acid or amino acid sequences themselves may be used in methods for diagnosing, preventing, or treating a neurodegenerative disorder, as described herein. Neurodegenerative defects, diseases, or disorders that may be treated using the polypeptides and polynucleotides described herein include, for example, Huntington's disease, Parkinson's disease, amyotrophic lateral sclerosis, or cerebellar ataxias.

Retinal Disorders

Retinal degenerative diseases, including retinitis pigmentosa and age-related macular degeneration, are a common cause of a visual defect. Understanding the genetic defects that underlie various retinal degenerative diseases may lead to improved diagnosis, prevention, and/or therapies. Zebrafish having a mutation in a CopZ1, VPSP18, Ribonucleotide Reductase R1 Class 1, Telomeric Repeat Factor 2, SIL, or ISWI/SNF2 nucleic acid or amino acid sequence display retinal degeneration. Accordingly, such mutant zebrafish may be used in screens to identify test compounds that affect retinal degeneration. In addition, CopZ1 nucleic acid and amino acid sequences may be used in methods to diagnose, prevent, or treat retinal degeneration, as described herein. Retinal defects, diseases, or disorders that may be treated using the polypeptides and polynucleotides described herein include, for example, retinitis pigmentosa, macular degeneration, Friedreich's ataxia, or Laurence-Moon syndrome, and pigmentation disorders, such as human pigmentary glaucoma, Wagner's disease, Cockayne's syndrome, Kearns-Sayre disease, Waardenburg syndrome, and Usher's syndrome.

Stroke

Stroke is a leading cause of death, killing approximately 160,000 Americans each year and is also the number one cause of adult disability. Given the prevalence of stroke, it is important to identify genetic mutations that may be used to assess genetic predisposition to stroke, as well as to identify drug targets for stroke therapies. Based on the phenotype of zebrafish having a mutation in a 1463, 904, or Mitochondrial Inner Membrane Translocating Protein (rTIM23) gene, and based on the homology of the Mitochondrial Inner Membrane Translocating (rTIM23) protein to LimpII, which interacts with an inhibitor of angiogenesis, 1463, 904, or Mitochondrial Inner Membrane Translocating Protein (rTIM23) nucleic acid and amino acid molecules may function in the development of vasculature in the brain and may be an important therapeutic target for stroke therapy. Accordingly, zebrafish mutant for a 1463, 904, or Mitochondrial Inner Membrane Translocating Protein (rTIM23) nucleic acid and amino acid molecule may be used in screens to identify compounds that may be used to diagnose, prevent, or treat stroke or other bleeding disorders in the brain. In addition, 1463, 904, or Mitochondrial Inner Membrane Translocating Protein (rTIM23) nucleic acid and amino acid molecules themselves may also be used to diagnose, prevent, or treat such disorders using the methods described herein.

Stem Cell Generation

The identification of genes that function in stem cell generation is important to organ regeneration. Mutants with defects in stem cell populations lack entire organs. Zebrafish with a mutation in a 1447 nucleic acid or amino acid sequence have no pancreas. Accordingly, such mutant zebrafish may be used in methods of identifying test compounds that affect organ regeneration. In addition, 1447 nucleic acid or amino acid molecules themselves may be used in methods of organ regeneration as described herein.

Pigmentation

Pigmentary glaucoma is a significant cause of human visual defects. In this disease, abnormally liberated iris pigment and cell debris enter the ocular drainage structures, leading to increased intraocular pressure (IOP) and glaucoma. In addition, abnormal pigment production and mutant melanosomal protein genes may contribute to human pigmentary glaucoma. Accordingly, therapeutic strategies designed to decrease pigment production may be beneficial in preventing or treating human pigmentary glaucoma. Therefore, nucleic acids and polypeptides that, when altered, result in pigmentation defects may be used in the diagnosis or treatment of visual defects, for instance, blindness due to human pigmentary glaucoma. Zebrafish that have mutations in a V-ATPase SFD Subunit, V-ATPase 16 kDa Proteolytic Subunit, CopZ1, V-ATPase Alpha Subunit, 1463, VPSP18, 297, Valyl-tRNA Synthase, Mitochondrial Inner Membrane Translocating Protein (rTIM23), or ARS2 nucleic acid or amino acid sequence have pigmentation defects. Consequently, such mutant zebrafish may be used in methods of identifying test compounds that may be useful in the diagnosis, prevention, or treatment of visual defects. In addition, V-ATPase SFD Subunit, V-ATPase16 kDa Proteolytic Subunit, CopZ1, V-ATPase Alpha Subunit, 1463, VPSP18, 297, Valyl-tRNA Synthase, Mitochondrial Inner Membrane Translocating Protein (rTIM23), or ARS2 nucleic acid and amino acid sequences themselves may be used in methods to treat visual defects, as described herein. Pigmentation defects, diseases, or disorders that may be prevented, treated, or diagnosed using the polypeptides and polynucleotides described herein include, for example, human pigmentary glaucoma, Wagner's disease, Cockayne's syndrome, Kearns-Sayre disease, Waardenburg syndrome, Usher's syndrome, or retinitis pigmentosa.

Pulmonary Disorders

The zebrafish swim bladder is thought to be a primitive lung. Consequently, zebrafish, for example, zebrafish having a mutation in a Fibroblast Isoform of the ADP/ATP Carrier Protein or TAFII-55 nucleic acid or amino acid sequence, with defective swim bladders may be used in screens to identify test compounds that may be useful in the diagnosis, prevention, or treatment of pulmonary disorders. In addition, Fibroblast Isoform of the ADP/ATP Carrier Protein and TAFII-55 nucleic acid and amino acid molecules may be used in the diagnosis, prevention, or treatment of pulmonary disorders using methods described herein. Pulmonary defects, diseases, or disorders that may be treated using the polypeptides and polynucleotides described herein include, for example, asthma or chronic bronchitis.

Movement Disorders

Movement defects of varying etiologies are prevalent in both children and adults. Understanding the etiology of the various movement disorders may lead to improved therapies. Zebrafish having a mutation in a Cad-1, Aryl Hydrocarbon Receptor Nuclear Translocator 2A, or 40S Ribosomal Protein S5, Neurogenin Related Protein 1, SIL, U1 Small Nuclear Ribonucleoprotein C, Ski Interacting Protein nucleic acid or amino acid sequence display motility defects. Accordingly, such mutant zebrafish may be used in methods to identify test compounds that affect motility. In addition, Cad-1, Aryl Hydrocarbon Receptor Nuclear Translocator 2A, or 40S Ribosomal Protein S5, Neurogenin Related Protein 1, SIL, U1 Small Nuclear Ribonucleoprotein C, or Ski Interacting Protein nucleic acid or amino acid sequences may be used in methods to diagnose, prevent, or treat human movement disorders. Movement defects, diseases, or disorders that may be treated using the polypeptides and polynucleotides described herein include, for example, Angelman's syndrome, tardive dyskinesia, spasticity, ataxias, or spinocerebellar ataxia; also neurodegenerative disorders, for example, Huntington's disease, or Parkinson's disease, Alzheimer's disease.

Somite Formation

Congenital myopathies are developmental disorders of muscle. These congenital defects include segmental amyoplasia, generalized amyoplasia, and congenital muscle fiber-type disproportion. Neonatal myotonic dystrophy is a maturational delay in muscle development. Zebrafish having a mutation in a 428, Smoothened, Glypican-6 or Knypek, or Denticleless, or 60S Ribosomal L35 nucleic acid or amino acid sequence exhibit abnormal somite formation. Accordingly, such zebrafish mutants may be used in methods of identifying test compounds that may be used to diagnose, prevent, or treat congenital muscle disorders. In addition, 428, 60S Ribosomal Protein L35, Smoothened, Glypican-6 or knypek, and Denticleless nucleic acid and amino acid sequences may be used in methods to diagnose, prevent, or treat congenital muscle disorders, as described herein. Muscle defects, diseases, or disorders that may be treated using the polypeptides and polynucleotides described herein include, for example, myotonia congenital, inclusion body myositis, muscular atrophy, or a congenital muscular dystrophy.

### Treatment

The nucleic acids and polypeptides of the present invention are useful for treating a variety of diseases and disorders, including proliferative disorders, bone, connective tissue, and cartilage formation disorders, cell death disorders, circulatory defects, craniofacial defects, visual defects, hearing disorders, diabetes, heart defects and diseases, kidney defects, limb formation defects, mental retardation, muscle defects, neurodegenerative disorders, retinal degeneration, stroke, stem cell population defects, pigmentation disorders, movement disorders, and somite formation defects. Nucleic acids and polypeptides of the present invention, for example, a 904, POU2, 40S Ribosomal Protein S18, U2AF, 954, Nrp-1, Cad-1, V-ATPase alpha subunit, V-ATPase SFD subunit, V-ATPase 16 kDa proteolytic subunit, 1463, VPSP18, pescadillo, HNF1-β/vHNF1, 60S Ribosomal Protein L35, 60S Ribosomal Protein L44, CopZ1, 215, 307, 572, 1116A, 1548, Casein Kinase 1 α, Nodal-Related or Squint, Smoothened, 429, 428, Spinster, Glypican-6 or Knypek, Ribonucleotide Reductase Protein R1 Class 1, Kinesin-Related Motor Protein EG5, 459, Wnt5 or Pipetail, Aryl Hydrocarbon Receptor Nuclear Translocator 2A, Vesicular Integral-Membrane Protein VIP 36, 299, 994, 1373, Denticleless, Ribonucleotide Reductase Protein R2, TCP-1 Alpha, Telomeric Repeat Factor 2, SIL, U1 Small Nuclear Ribonucleoprotein C, Ski Interacting Protein, 297, TCP-1 Complex Gamma Chain, Small Nuclear Ribonucleoprotein D1, DNA Polymerase Epsilon Subunit B, 821-02, 1045, 1055-01, Spliceosome Associated Protein 49, DNA Replication Licensing Factor MCM7, DEAD-Box RNA Helicase (DEAD5 or DEAD19), 1581, Cyclin A2, ISWI/SNF2, Chromosomal Assembly Protein C (XCAP-C), DNA Replication Licensing Factor MCM2, DNA Replication Licensing Factor MCM3, Valyl-tRNA Synthase, 40S Ribosomal Protein S5, TCP-1 Beta, TCP-1 Eta, Translation Elongation Factor eEF1 Alpha, 1257, 60S Ribosomal Protein L24, Non-Muscle Adenylosuccinate Synthase, Nuclear Cap Binding Protein Subunit 2, Ornithine Decarboxylase, Protein Phosphatase 1 Nuclear Targeting Subunit (PNUTS), Mitochondrial Inner Membrane Translocating Protein (rTIM23), 1447, ARS2, Sec61 Alpha, BAF53a, Histone Deacetylase, Fibroblast Isoform of the ADP/ATP Carrier Protein, or a TAFII-55 nucleic or amino acid sequence, may be administered by any of a variety of routes known to those skilled in the art, such as, for example, by intraperitoneal, subcutaneous, parenteral, intravenous, intramuscular, or subdermal injection. However, the nucleic acids or polypeptides may also be administered as an aerosol, as well as orally, nasally, or topically. Appropriate carriers or diluents for, as well as what is essential for the preparation of a pharmaceutical composition are described, e.g., in Remington's Pharmaceutical Sciences (18^{th} edition), ed. A. Gennaro, 1990, Mack Publishing Company, Easton, Pa., a standard reference book in this field.

Formulations for parenteral administration may, for example, contain excipients, sterile water, or saline. For inhalation, formulations may contain excipients, for example, lactose. Aqueous solutions may be used for administration in the form of nasal drops, or as a gel for topical administration. The exact dosage used will depend on the severity of the condition or the general health of the patient and the route of administration. One skilled in the art would know how to determine and adjust the dosage as required. These nucleic acids or polypeptides may be administered once, or they may be repeatedly administered as part of a regular treatment regimen over a period of time.

In addition, the invention provides methods of gene therapy by targeting, for example, a nucleic acid sequence descibed herein. Such a nucleic acid sequence may be introduced into an abnormal cell, for example, by using liposome-based transfection techniques, to treat a disorder (Units 9.1-9.4, Ausubel et al., Current Protocols in Molecular Biology, John Wiley & Sons, New York, 1995). Such DNA constructs may also be introduced into mammalian cells using an adenovirus, or retroviral or vaccinia viral vectors (Units 9.10 and 16.15-16.19, Ausubel et al., Current Protocols in Molecular Biology, John Wiley & Sons, New York, 1995). These standard methods of introducing DNA into cells are applicable to a variety of cell-types.

For example, recombinant adenoviral vectors offer several significant advantages for gene transfer. The viruses can be prepared at extremely high titer, infect non-replicating cells, and confer high-efficiency and high-level transduction of target cells in vivo after directed injection or perfusion. Either directed injection or perfusion would be appropriate for delivery of vectors containing a therapeutic gene in a clinical setting. When a viral vector is used to administer the nucleic acids of the invention, standard concentrations include, for example, 10², 10³, 10⁴, 10⁵, or 10⁶ plaque forming units (pfu)/animal, in a pharmacologically acceptable carrier.

In animal models, adenoviral gene transfer has generally been found to mediate high-level expression for approximately one week. The duration of transgene expression may be prolonged, and ectopic expression reduced, by using tissue-specific promoters. Other improvements in the molecular engineering of the adenoviral vector itself have produced more sustained transgene expression and less inflammation. This is seen with so-called “second generation” vectors harboring specific mutations in additional early adenoviral genes and “gutless” vectors in which virtually all the viral genes are deleted utilizing a Cre-Lox strategy (Engelhardt, et al., Proc. Natl. Acad. Sci. USA 91:6196-6200, 1994; Kochanek, et al., Proc. Natl. Acad. Sci. USA 93:5731-5736, 1996).

In addition, recombinant adeno-associated viruses (rAAV), derived from non-pathogenic parvoviruses, may be used to express a target gene as these vectors evoke almost no cellular immune response, and produce transgene expression lasting months in most systems. Incorporation of a tissue-specific promoter is, again, beneficial. Furthermore, besides adenovirus vectors and rAAVs, other vectors and techniques are known in the art, for example, those described by Wattanapitayakul and Bauer (Biomed. Pharmacother. 54:487-504, 2000), and citations therein.

A vector carrying a therapeutic gene can be delivered to the target organ through in vivo perfusion by injecting the vector into the target organ, or into blood vessels supplying this organ (e.g., for the liver, the portal vein (Tada, et al., Liver Transpl. Surg. 4:78-88, 1998) could be used, or in the case of leukemia, the blood itself may be the delivery target.

RNA Interference

Alternatively, a naturally-occurring nucleic acid sequence corresponding to any of the nucleic acid sequences on the invention may be inactivated using RNA interference (“RNAi”). RNAi is a form of post-transcriptional gene silencing initiated by the introduction of double-stranded RNA (dsRNA) or antisense RNA. RNAi was first characterized in C. elegans, where many expressed genes are subject to inactivation by RNAi (Fire et al., Nature 391:806-11, 1998; Fraser et al., Nature 408:325-30, 2000). This effect has also been observed in a variety of other organisms including Drosophila melanogaster and mammals.

In particular, short twenty-one to twenty-five nucleotide double-stranded RNAs are effective at down-regulating gene expression in nematodes (Zamore et al., Cell 101: 25-33) and in mammalian tissue culture cell lines (Elbashir et al., Nature 411:494-498, 2001, hereby incorporated by reference). The further therapeutic effectiveness of this approach in mammals was demonstrated in vivo by McCaffrey et al. (Nature 418:38-39, 2002). The nucleic acid sequence of a gene descibed herein can be used to design small interfering RNAs (“siRNAs”) that will inactivate a naturally-occurring gene that contains the specific 21 to 25 nucleotide RNA sequences used.

Test Compounds

Compounds that may be tested for the ability to modulate the expression of target genes, or of their gene products, can be from natural as well as synthetic sources. Those skilled in the field or drug discovery and development will understand that the precise source of test extracts or compounds is not critical to the methods of the invention. Examples of such extracts or compounds include, but are not limited to, plant-based, fungal-based, prokaryotic-based, or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-based, lipid-based, peptide-based, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceanographics Institute (Ft. Pierce, Fla.), and Pharmamar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

A test compound that modulates the expression of a target gene, or its encoded protein, may be used to treat any of the diseases and disorders described above.

Diagnosis

The methods of the present invention can be used to diagnose a disease or disorder, or the propensitiy to develop a disease or disorder.

A genetic lesion in a candidate gene may be identified in a biological sample obtained from a patient using a variety of methods available to those skilled in the art. Generally, these techniques involve PCR amplification of nucleic acid from the patient sample, followed by identification of the genetic lesion by altered hybridization, aberrant electrophoretic gel migration, restriction fragment length polymorphism (“RFLP”) analysis, binding or cleavage mediated by mismatch binding proteins, or direct nucleic acid sequencing. Any of these techniques may be used to facilitate detection of a genetic lesion in a candidate gene, and each is well known in the art; examples of particular techniques are described, without limitation, in Orita et al. (Proc. Natl. Acad. Sci. USA 86:2766-2770, 1989) and Sheffield et al. (Proc. Natl. Acad. Sci. USA 86:232-236, 1989). Furthermore, expression of the candidate gene in a biological sample (e.g., a biopsy) may be monitored by standard Northern blot analysis or may be aided by PCR (see, e.g., Ausubel et al., Current Protocols in Molecular Biology, John Wiley & Sons, New York, 2000; PCR Technology: Principles and Applications for DNA Amplification, H. A. Ehrlich, Ed., Stockton Press, NY; and Yap et al., Nucl. Acids. Res. 19:4294, 1991).

Once a genetic lesion is identified using the methods of the invention (as is described above), the genetic lesion is analyzed for association with a particular disease or disorder, for example, a proliferative disorder or an increased risk of developing such a disorder.

Furthermore, antibodies against a protein produced by the gene included in the genetic lesion, for example, a protein encoded by any of the nucleic acid sequences described herein, may be used to detect altered expression levels of the protein, including a lack of expression, or a change in its mobility on a gel, indicating a change in structure or size. In addition, antibodies may be used for detecting an alteration in the expression pattern or the sub-cellular localization of the protein. Such antibodies include ones that recognize both a wild-type and a mutant protein, as well as ones that are specific for either the wild-type or an altered form of the protein. If desired, monoclonal antibodies may also be prepared using the proteins described above and standard hybridoma technology (see, e.g., Kohler et al., Nature 256:495, 1975); Kohler et al., Eur. J. Immunol. 6:511, 1976); Kohler et al., Eur. J. Immunol. 6:292, 1976); Hammerling et al., In Monoclonal Antibodies and T Cell Hybridomas, Elsevier, New York, N.Y., 1981; Ausubel et al., Current Protocols in Molecular Biology, John Wiley & Sons, New York, 2000)). The specificicity of a monoclonal antibody may be tested by Western blot or immunoprecipitation analysis (by the methods described in, for example, Ausubel et al. (Current Protocols in Molecular Biology, John Wiley & Sons, New York, 2000).

Antibodies used in the methods of the invention may be produced using amino acid sequences that do not reside within highly conserved regions, and that appear likely to be antigenic, as analyzed by criteria such as those provided by the Peptide Structure Program (Genetics Computer Group Sequence Analysis Package, Program Manual for the GCG Package, Version 7, 1991) using the algorithm of Jameson and Wolf (CABIOS 4:181, 1988). These fragments can be generated by standard techniques, e.g., by the PCR, and cloned into the pGEX expression vector (Ausubel et al., Current Protocols in Molecular Biology, John Wiley & Sons, New York, 2000). GST fusion proteins are expressed in E. coli and purified using a glutathione agarose affinity matrix as described in Ausubel et al. (Current Protocols in Molecular Biology, John Wiley & Sons, New York, 2000).

Use of Transgenic and Knockout Animals in Diagnosis

The disclosed transgenic and knock-out animals may be used as research tools to determine genetic and physiological features of a disease or disorder, and for identifying compounds that can affect such diseases or disorders. For example, a zebrafish embryo, for example, a fertilized egg, or a one-day old embryo, that is mutant, either homozygous or heterozygous, for a 904, POU2, 40S Ribosomal Protein S18, U2AF, 954, Nrp-1, Cad-1, V-ATPase Alpha Subunit, V-ATPase SFD Subunit, V-ATPase 16 kDa Proteolytic Subunit, 1463, VPSP18, Pescadillo, HNF1-β/vHNF1, 60S Ribosomal Protein L35, 60S Ribosomal Protein L44, CopZ1, 215, 307, 572, 1116A, 1548, Casein Kinase 1 a, Nodal-Related or Squint, Smoothened, 429, 428, Spinster, Glypican-6 or Knypek, Ribonucleotide Reductase Protein R1 Class 1, Kinesin-Related Motor Protein EG5, 459, Wnt5 or Pipetail, Aryl Hydrocarbon Receptor Nuclear Translocator 2A, Vesicular Integral-Membrane Protein VIP 36, 299, 994, 1373, Denticleless, Ribonucleotide Reductase Protein R2, TCP-1 Alpha, Telomeric Repeat Factor 2, SIL, U1 Small Nuclear Ribonucleoprotein C, Ski Interacting Protein, 297, TCP-1 Complex Gamma Chain, Small Nuclear Ribonucleoprotein D1, DNA Polymerase Epsilon Subunit B, 821-02, 1045, 1055-01, Spliceosome Associated Protein 49, DNA Replication Licensing Factor MCM7, DEAD-Box RNA Helicase (DEAD5 or DEAD19), 1581, Cyclin A2, ISWI/SNF2, Chromosomal Assembly Protein C (XCAP-C), DNA Replication Licensing Factor MCM2, DNA Replication Licensing Factor MCM3, Valyl-tRNA Synthase, 40S Ribosomal Protein S5, TCP-1 Beta, TCP-1 Eta, Translation Elongation Factor eEF1 Alpha, 1257, 60S Ribosomal Protein L24, Non-Muscle Adenylosuccinate Synthase, Nuclear Cap Binding Protein Subunit 2, Ornithine Decarboxylase, Protein Phosphatase 1 Nuclear Targeting Subunit (PNUTS), Mitochondrial Inner Membrane Translocating Protein (rTIM23), 1447, ARS2, Sec61 Alpha, BAF53a, Histone Deacetylase, Fibroblast Isoform of the ADP/ATP Carrier Protein, or a TAFII-55 nucleic acid or amino acid sequence may be contacted with a test compound described herein, followed by observing the development of this embryo. In this manner, test compounds can be identified that decrease or eliminate the developmental abnormalities and such test compounds would serve as candidate compounds for treating a related human disease or disorder.

Knockout animals also include animals where this normal gene has been inactivated or removed and replaced with a known polymorphic or other mutant allele of this gene. These animals can serve as a model system for the risk of acquiring a disease that is associated with a particular allele. In general, the method of identifying markers associated with a proliferative disorder, involves comparing the presence, absence, or level of expression of genes, either at the RNA level or at the protein level, in tissue from a transgenic or knock-out animal and in tissue from a matching non-transgenic or knock-out animal. Standard techniques for detecting RNA expression, e.g., by Northern blotting, or protein expression, e.g., by Western blotting, are well known in the art. Differences between animals such as the presence, absence, or level of expression of a gene indicate that the expression of the gene is a marker associated with a disorder. Identification of such markers would be useful since they are possible therapeutic targets. Identification of markers can take several forms.

One method by which molecular markers may be identified is by use of directed screens. Patterns of accumulation of a variety of molecules can be surveyed using immunohistochemical methods. Screens directed at analyzing expression of specific genes or groups of molecules implicated in pathogenesis can be continued during the life of the transgenic or knockout animal. Expression can be monitored by immunohistochemistry as well as by protein and RNA blotting techniques.

Alternatively, molecular markers may be identified using genomic screens. For example, tissue can be recovered from young transgenic or knockout animals and older transgenic or knockout animals, and compared with similar material recovered from age-matched normal littermate controls to catalog genes that are induced or repressed as disease is initiated, and as disease progresses to its final stages. These surveys will generally include cellular populations present in the affected tissue.

This analysis can also be extended to include an assessment of the effects of various treatments on differential gene expression (“DGE”). The information derived from the surveys of DGE can ultimately be correlated with disease initiation and progression in the transgenic or knockout animals.

To assess the effectiveness of a treatment paradigm, a transgene, such as a mutant of any of the nucleic acid sequences described herein, may be conditionally expressed (e.g., in a tetracycline sensitive manner). For example, the promoter for this gene may contain a sequence that is regulated by tetracycline and expression of the gene product ceases when tetracycline is administered to the mouse. In this example, a tetracycline-binding operator, tetO, is regulated by the addition of tetracycline, or an analog thereof, to the organism's water or diet. The tetO may be operably-linked to a coding region, for example, a wild-type or mutant nucleic acid sequence described herein. The system also may include a tetracycline transactivator (“tTA”), which contains a DNA binding domain that is capable of binding the tetO as well as a polypeptide capable of repressing transcription from the tetO (e.g., the tetracycline repressor (“tetR”)), and may be further coupled to a transcriptional activation domain (e.g., VP16). When the tTA binds to the tetO sequences, in the absence of tetracycline, transcription of the target gene is activated. However, binding of tetracycline to the tTA prevents activation. Thus, a gene operably-linked to a tetO is expressed in the absence of tetracycline and is repressed in its presence. Alternatively, this system could be modified such that a gene is expressed in the presence of tetracycline and repressed in its absence. Tetracycline regulatable systems are well known to those skilled in the art and are described in, for example, WO 94/29442, WO 96/40892, WO 96/01313, and Yamamoto et al. (Cell 101:57-66, 2000).

In addition, the knockout organism may be a conditional, i.e., somatic, knockout. For example, FRT sequences may be introduced into the organism so that they flank the gene of interest. Transient or continuous expression of the FLP protein may then be used to induce site-directed recombination, resulting in the excision of the gene of interest. The use of the FLP/FRT system is well established in the art and is described in, for example, U.S. Pat. No. 5,527,695, and in Lyznik et al. (Nucleic Acid Research 24:3784-3789, 1996).

Conditional, i.e., somatic knockout organisms may also be produced using the Cre-lox recombination system. Cre is an enzyme that excises DNA between two recognition sites termed loxP. The cre transgene may be under the control of an inducible, developmentally regulated, tissue specific, or cell-type specific promoter. In the presence of Cre, the gene, for example a nucleic acid sequence described herein, flanked by loxP sites is excised, generating a knockout. This system is described, for example, in Kilby et al. (Trends in Genetics 9:413-421, 1993).

Particularly desirable is a mouse model wherein an altered nucleic acid sequence described herein is expressed in specific cells of the transgenic mouse such that the transgenic mouse develops a disease or disorder. In addition, cell lines from these mice may be established by methods standard in the art.

Construction of transgenes can be accomplished using any suitable genetic engineering technique, such as those described in Ausubel et al. (Current Protocols in Molecular Biology, John Wiley & Sons, New York, 2000). Many techniques of transgene construction and of expression constructs for transfection or transformation in general are known and may be used for the disclosed constructs.

One skilled in the art will appreciate that a promoter is chosen that directs expression of the chosen gene in the tissue in which a disease or disorder is expected to develop. For example, as noted above, any promoter that regulates expression of a nucleic acid sequence described herein can be used in the expression constructs of the present invention. One skilled in the art would be aware that the modular nature of transcriptional regulatory elements and the absence of position-dependence of the function of some regulatory elements, such as enhancers, make modifications such as, for example, rearrangements, deletions of some elements or extraneous sequences, and insertion of heterologous elements possible. Numerous techniques are available for dissecting the regulatory elements of genes to determine their location and function. Such information can be used to direct modification of the elements, if desired. It is desirable, however, that an intact region of the transcriptional regulatory elements of a gene is used. Once a suitable transgene construct has been made, any suitable technique for introducing this construct into embryonic cells can be used.

Animals suitable for transgenic experiments can be obtained from standard commercial sources such as Taconic (Germantown, N.Y.). Many strains are suitable, but Swiss Webster (Taconic) female mice are desirable for embryo retrieval and transfer. B6D2F (Taconic) males can be used for mating and vasectomized Swiss Webster studs can be used to stimulate pseudopregnancy. Vasectomized mice and rats are publicly available from the above-mentioned suppliers. However, one skilled in the art would also know how to make a transgenic mouse or rat. An example of a protocol that can be used to produce a transgenic animal is provided below.

Production of Transgenic Mice and Rats

The following is but one desirable means of producing transgenic mice. This general protocol may be modified by those skilled in the art.

Female mice six weeks of age are induced to superovulate with a 5 IU injection (0.1 cc, IP) of pregnant mare serum gonadotropin (PMSG; Sigma) followed 48 hours later by a 5 IU injection (0.1 cc, IP) of human chorionic gonadotropin (hCG, Sigma). Females are placed together with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA, Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with humidified atmosphere at 5% CO₂, 95% air until the time of injection. Embryos can be implanted at the two-cell stage.

Randomly cycling adult female mice are paired with vasectomized males. Swiss Webster or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos are transferred. After the transferring the embryos, the incision is closed by two sutures.

A desirable procedure for generating transgenic rats is similar to that described above for mice (Hammer et al., Cell 63:1099-112, 1990). For example, thirty-day old female rats are given a subcutaneous injection of 20 IU of PMSG (0.1 cc) and 48 hours later each female placed with a proven, fertile male. At the same time, 40-80 day old females are placed in cages with vasectomized males. These will provide the foster mothers for embryo transfer. The next morning females are checked for vaginal plugs. Females who have mated with vasectomized males are held aside until the time of transfer. Donor females that have mated are sacrificed (CO₂ asphyxiation) and their oviducts removed, placed in DPBA (Dulbecco's phosphate buffered saline) with 0.5% BSA and the embryos collected. Cumulus cells surrounding the embryos are removed with hyaluronidase (1 mg/ml). The embryos are then washed and placed in EBSs (Earle's balanced salt solution) containing 0.5% BSA in a 37.5° C. incubator until the time of microinjection.

Once the embryos are injected, the live embryos are moved to DPBS for transfer into foster mothers. The foster mothers are anesthetized with ketamine (40 mg/kg, IP) and xulazine (5 mg/kg, IP). A dorsal midline incision is made through the skin and the ovary and oviduct are exposed by an incision through the muscle layer directly over the ovary. The ovarian bursa is torn, the embryos are picked up into the transfer pipet, and the tip of the transfer pipet is inserted into the infundibulum. Approximately 10 to 12 embryos are transferred into each rat oviduct through the infundibulum. The incision is then closed with sutures, and the foster mothers are housed singly.

Generation of Knockout Mice

The following is but one example for the generation of a knockout mouse and the protocol may be readily adapted or modified by those skilled in the art.

Embryonic stem cells (ES), for example, 10⁷ AB1 cells, may be electroporated with 25 μg targeting construct in 0.9 ml PBS using a Bio-Rad Gene Pulser (500μF, 230 V). The cells may then be plated on one or two 10-cm plates containing a monolayer of irradiated STO feeder cells. Twenty-four hours later, they may be subjected to G418 selection (350 μg/ml, Gibco) for 9 days. Resistant clones may then be analyzed by Southern blotting after Hind III digestion, using a probe specific to the targeting construct. Positive clones are expanded and injected into C57BL/6 blastocysts. Male chimeras may be back-crossed to C57BL/6 females. Heterozygotes may be identified by Southern blotting and intercrossed to generate homozygotes.

The targeting construct may result in the disruption of the gene of interest, e.g., by insertion of a heterologous sequence containing stop codons, or the construct may be used to replace the wild-type gene with a mutant form of the same gene, e.g., a “knock-in.” Furthermore, the targeting construct may contain a sequence that allows for conditional expression of the gene of interest. For example, a sequence may be inserted into the gene of interest that results in the protein not being expressed in the presence of tetracycline. Such conditional expression of a gene is described in, for example, Yamamoto et al. (Cell 101:57-66, 2000)).

1. An isolated nucleic acid molecule comprising a nucleic acid sequence having at least 75% nucleic acid sequence identity to the 459 nucleic acid sequence of SEQ ID NO:59 over at least 600 contiguous nucleic acids, wherein said nucleic acid molecule functions in kidney development.

2. The isolated nucleic acid molecule of claim 1, wherein said nucleic acid molecule comprises the sequence of SEQ ID NO:59.

3. The isolated nucleic acid molecule of claim 1, wherein said nucleic acid molecule is a human or mouse nucleic acid molecule.

4. The nucleic acid molecule of claim 1, wherein said nucleic acid molecule is a zebrafish nucleic acid molecule.

5. A vector comprising the isolated nucleic acid molecule of claim 1.

6. A cell comprising the vector of claim 5.

7. The isolated nucleic acid molecule of claim 1, wherein said nucleic acid molecule further comprises a viral insertion at a nucleotide corresponding to nucleotide 210 of SEQ ID NO:59.

8. A zebrafish comprising the nucleic acid molecule of claim 7.

9. An isolated polypeptide comprising an amino acid sequence having at least 75% sequence identity to the 459 amino acid sequence of SEQ ID NO:60 over at least 250 contiguous amino acids, wherein said polypeptide functions in kidney development.

10. The isolated polypeptide of claim 9, wherein said polypeptide comprises the sequence of SEQ ID NO:60.

11. A method of treating or preventing a kidney disorder in an organism, said method comprising the step of contacting said organism with a therapeutically effective amount of a nucleic acid comprising the nucleic acid of claim 1, or its complement, wherein the nucleic acid is sufficient to elicit an alteration in expression of a 459 nucleic acid sequence in said organism, and wherein said alteration in the level of expression treats or prevents a kidney disorder.

12. The method of claim 11, wherein said nucleic acid is a cDNA or an mRNA molecule and said contacting results in an increase in expression of the polypeptide encoded by a nucleic acid sequence comprising the nucleic acid sequence of SEQ ID NO:59.

13. The method of claim 11, wherein said nucleic acid is a double-stranded RNA molecule and said contacting leads to a decrease in expression, or inhibits biological activity, of a nucleic acid sequence comprising SEQ ID NO:59.

14. The method of claim 11, wherein said nucleic acid is an anti-sense RNA molecule, and said contacting leads to a decrease in expression, or inhibits biological activity, of a nucleic acid sequence comprising SEQ ID NO:59.

15. A method for diagnosing a kidney disorder or the propensity to develop a kidney disorder in an organism, said method comprising detecting an alteration in the level of 459 polypeptide expression in a sample derived from a first organism and comparing the level of expression to that of a 459 polypeptide in a sample derived from a second, control organism, wherein an alteration in the level of expression or activity of said 459 polypeptide in said first organism relative to said second organism is indicative of said first organism having or having a propensity to develop a kidney disorder.

16. The method of claim 15, wherein said 459 polypeptide comprises the amino acid sequence of SEQ ID NO:60.
